(19) United States
(12) Patent Application Publication
(10) Pub. No.: US 2024/0390496 A1
(43) Pub. Date: Nov. 28, 2024

(54) SINGLE-CHAIN AND MULTI-CHAIN SYNTHETIC ANTIGEN RECEPTORS FOR DIVERSE IMMUNE CELLS

(71) Applicant: UNIVERSITY OF SOUTHERN CALIFORNIA, Los Angeles, CA (US)

(72) Inventor: Preet M. CHAUDHARY, Toluca Lake, CA (US)

(21) Appl. No.: 18/275,957

(22) PCT Filed: Feb. 21, 2022
(86) PCT No.: PCT/US2022/017177

§ 371 Date: Jan. 25, 2024

(51) Int. Cl. A61K 39/00; C07K 14/725; C07K 16/28

(52) U.S. Cl. CPC A61K 39/464412; A61K 39/4613; A61K 39/4631; C07K 14/7051; C07K 16/2803; C07K 2317/732

(57) ABSTRACT

The disclosure provides single-chain and multi-chain synthetic antigen receptors, methods of making such synthetic antigen receptors and uses thereof for the treatment of diseases and disorder.

### CROSS REFERENCE TO RELATED APPLICATIONS

The application claims priority under 35 U.S.C. § 119 to U.S. Provisional Application Ser. No. 63/151,421, filed Feb. 19, 2021 and U.S. Provisional Application No. 63/245,181, filed Sep. 16, 2021, the disclosures of which are incorporated herein by reference for all purposes.

### TECHNICAL FIELD

The present disclosure relates to the field of biotechnology, and more specifically, to single-chain and multi-chain synthetic antigen receptors.

### INCORPORATION BY REFERENCE OF SEQUENCE LISTING

Accompanying this filing is a Sequence Listing entitled “NK-DNA-PRT-1-11231_ST25.txt”, created on Feb. 21, 2022 and having 34,352,306 bytes of data, machine formatted on IBM-PC, MS-Windows operating system. The sequence listing is hereby incorporated herein by reference in its entirety for all purposes.

### BACKGROUND

CARs are synthetic immune receptors, which can redirect T cells to selectively kill tumor cells. Despite the success with CAR-T cells, there are several limitations to this approach, including toxicities such as “Cytokine release syndrome’ (CRS) and neurotoxicities.

In contrast to CAR-T, NK (natural killer) cells are naturally endowed with cytolytic functions and antiviral immunity but lack TCRs that could cause GvHD. CAR-NK cells, in contrast to CAR-T, are also less likely to result in excessive cytokine production.

The 2^{nd} generation CARs in current clinical use are a fusion of several different polypeptides. For example, Kymriah comprises of a murine scFv (FMC63), human CD8 hinge and transmembrane domains, a human 4-1BB costimulatory domain and a human CD3z activation domain. These domains have been stitched together in somewhat arbitrary manner and the resulting construct suffers from several problems, such as non-specific aggregation, tonic signaling and lack of physiological regulation.

Without wishing to be bound by theory, these problems are likely to be compounded when scFvs are used to design CARs having bispecific, bivalent or biparatopic antigen binding moieties.

Another major limitation of most of the above next generation SAR designs is that they are primarily active in T cells and are not functional in other immune cells, such as NK cells, monocytes/macrophages, dendritic cells and neutrophils.

To overcome some of the design limitation of conventional 2^{nd} generation CARs, several alternative designs, collectively termed next generation CARs, have been described, including Ab-TCR (WO 2017/070608 A1 incorporated herein by reference), TCR receptor fusion proteins or TFP (WO 2016/187349 A1 incorporated herein by reference), Synthetic Immune Receptors (SIRs) (see, WO 2018/102795 A1, incorporated herein by reference), Tri-functional T cell antigen coupler (Tri-TAC) (see, WO 2015/117229 A1, incorporated herein by reference). These alternative CAR designs, in general, lack a co-stimulatory domain.

### SUMMARY

The disclosure provides unispecific, bispecific, multispecific and universal next generation SAR designs.

The disclosure provides Synthetic antigen-receptors (SARs) with specific configuration of extracellular, transmembrane and cytosolic domains that when expressed in a immune-cell (e.g., T cell NK cell, NKT cell, monocyte, macrophage, neutrophil etc.), demonstrate improved immune-cell activation, target cell killing, cytokine secretion (e.g., IL-2, interferon-gamma, and TNFa) and in vivo activity as compared to a immune-cell that expresses a conventional chimeric antigen receptor (CAR), e.g., a second generation CAR that expresses a CD3z activation domain and a 41BB or CD28 costimulatory domain.

The disclosure also provides novel accessory modules that can be co-expressed with the SARs of the disclosure. The disclosure provides vectors comprising nucleic acids encoding polypeptides for a) membrane anchored low-affinity variants of cytokines (e.g., IL-2 and/or IL-15); b) membrane anchored cytokines with epitope tags; and c) multi-purpose gene switches that serve suicide, survival and marker functions.

The disclosure provides a method of producing a cell that expresses any one or more of the accessory modules with any one or more of the single chain, or multi-chain SARs of the disclosure. The accessory modules can be expressed in a cell without a SAR. The disclosure also provides optimized vectors with short promoters and internal ribosomal sites that are optimized for expression of the accessory modules and/or SARs of the disclosure.

In one aspect, the disclosure relates to single chain novel next generation SAR designs that provide physiological signaling. More importantly, in another aspect, the disclosure relates to multi-chain novel next generation SAR designs.

In another aspect, the disclosure relates to novel next generation synthetic antigen receptor (SAR) designs that are active in a variety of immune cells, including T cells, NKT cells, NK cells, monocytes/macrophages and neutrophils etc. In another aspect, the disclosure relates to novel SAR design, called a universal TCR-SAR (or uTCR-SAR), that confers T cell receptor like antigen binding specificity to any cell.

The disclosure also provides a non-T cell, including any cell, with T cell like binding properties, including the ability to bind to a peptide antigen in association with an MHC (or HLA) molecule. The disclosure provides a general method for generating such cells and their use in the treatment of various diseases.

The disclosure also provides a multipurpose switch that can be used in adoptive cell therapy for providing cell survival, detection, tracking, enrichment, selection and elimination functions.

The disclosure also provides a universal method for generation of a chimeric fusion protein involving a Type I and Type II transmembrane protein. The method can be used to generate synthetic antigen receptors incorporating the antigen binding domain of a Type I protein and the cytosolic, transmembrane, hinge and/or extracellular antigen binding domain of a Type II protein.

Also provided herein are nucleic acids that encode any of the single chain, double-chain or multi-chain SARs and/or accessory modules described herein. Also provided herein are sets of nucleic acids that together encode any of the single chain, double-chain and multi-chain SARs and/or accessory modules described herein.

In some embodiments, according to any of the SARs (such as isolated SARs) described above, there is provided an effector cell (e.g., T cell, NK cell, macrophage, iPSC etc.) presenting on its surface the SAR, wherein the effector cell comprises one or more vectors with one or more promoters comprising one or more nucleic acids encoding the one or more polypeptide chains of the SAR and/or the optional accessory modules.

Also provided herein are mammalian cells that include any of the nucleic acids described herein that encode any of the single chain, double chain and multi-chain SARs and/or accessory modules described herein. Also provided herein are mammalian cells that include any of the sets of nucleic acids described herein that together encode any of the single chain, double chain and multi-chain SARs and/or accessory modules described herein.

In some embodiments, the disclosure provides that, in contrast to a TCR, a SAR of the disclosure can be expressed in any mammalian cell and be functionally active. In an embodiment, the mammalian cells is a T cell, NK cell, macrophage, granulocyte etc. In some embodiments of any of the mammalian cells described herein, the mammalian cell is selected from the group of: a CD8⁺ T cell, a CD4⁺ T cell, a memory T cell, naïve T cell, T stem cell, a Treg cell, natural killer T (NKT) cell, iNKT (innatet natural killer cell), NK cell, g-NK cell, memory like NK cells, cytokine induced killer cell (CIK), iPSC-derived NK cell, α/β T cell, γ/δ T cell, iPSC-derived T cell, B cell, a macrophage/monocyte, iPSC. In some embodiments of any of the mammalian cells described herein, the mammalian cell is selected from the group of: iPSC (induced pluripotent stem cell or embryonic stem cell or hematopoietic stem cell that can give rise to an immune effector cell (e.g., a T cell, NK cell or NKT cell). In some embodiment, the mammalian cell is an immortalized cell line, such as NK92, NK92MI, YTS or a derivative thereof. In some embodiments of any of the mammalian cells described herein, the mammalian cell is a mammalian cell obtained from a subject. In some embodiments of any of the mammalian cells described herein, the subject is diagnosed or identified as having a cancer. In some embodiments of any of the mammalian cells described herein, the subject is human. In some embodiment, the cell is autologous. In some embodiment, the cell is allogeneic.

Also, provided herein are single chain and multichain TCRs that can be functionally expressed in cells other than T cells including, but not limited to, NK cells, monocytes, macrophages, dendritic cells and granulocytes.

Also provided herein are pharmaceutical compositions that include any of the mammalian cells described herein and a pharmaceutically acceptable carrier. Also provided herein are kits that include any of the pharmaceutical compositions described herein.

Also provided herein are pharmaceutical compositions that include any of the nucleic acids described herein that encode any of the single chain, double chain and multi-chain SARs and/or accessory modules described herein, or any of the sets of nucleic acids described herein that together encode any of the single chain, double chain and multi chain SARs and/or accessory modules described herein, and a pharmaceutically acceptable carrier. Also provided herein are kits that include any of the pharmaceutical compositions described herein.

In some embodiments, there is provided a method of killing a target cell presenting one or more target antigens, comprising contacting the target cell with an effector cell expressing a SAR according to any of the SARs (such as isolated SARs) described above, wherein the SAR specifically binds to one or more target antigens.

In some embodiments, according to any of the methods of killing a target cell described above, the contacting is in vivo. In some embodiments, the contacting is in vitro.

In some embodiment, there are provided methods for detection, isolation, purification, expansion, enrichment and elimination of cells expressing any of the SAR described herein.

Also provided herein are methods of generating a cell expressing a single chain, double chain and multi-chain SAR and/or accessory modules that include introducing into a mammalian cell any of the nucleic acids described herein that encode any of the SARs and accessory modules described herein, or any of the sets of nucleic acids described herein that encode any of the multi-chain SARs described herein. In some embodiments of any of the methods described herein, the mammalian cell is a human cell. In some embodiments of any of the methods described herein, the mammalian cell is a cell selected from the group consisting of: a CD8⁺ T cell, a CD4⁺ T cell, a memory T cell, a Treg cell, natural killer T cell, B cell, NK cells, and a macrophage/monocyte. In some embodiments of any of the methods described herein, the mammalian cell is a mammalian cell obtained from a subject. In some embodiments of any of the methods described herein, the subject is diagnosed or identified as having a cancer. Some embodiments of any of the methods described herein further include, after the introducing step, culturing the cell in a liquid culture medium. Some embodiments of any of the methods described herein further include, before the introducing step, obtaining the mammalian cell from the subject.

Also provided herein are methods of treating a disease (e.g., cancer, infection, allergy, immune disorder etc.) in a subject that include administering a therapeutically effective amount of any of the mammalian cell described herein to the subject. Some embodiments of any of the methods described herein further include, prior to the administering step, obtaining an initial cell from the subject; and introducing any of the nucleic acids descried herein that encode any of the single chain, double chain, multi-chain SARs and/or accessory modules described herein or any of the sets of nucleic acids described herein that together encode any of the single chain, double chain, multi-chain SARs and/or accessory modules described herein into the initial cell, to yield the mammalian cell that is administered to the subject. Some embodiments of any of the methods described herein further include, between the introducing step and the administering step, a step of culturing the cell that is administered to the subject in a liquid culture medium. In some embodiments of any of the methods described herein, the subject is human.

In some embodiments of any of the single-chain SARs described herein, the heterologous antigen-binding domain is selected from the group of: an antibody, an antibody fragment (vL, vH, Fab etc.) a scFv, a (scFv)₂, a VHH domain, FHVH (a fully human vH domain), a single domain antibody, a non-immunoglobulin antigen binding scaffold (e.g., Centyrin, affibody, ZIP domain, an adaptor etc.), a VNAR domain, a ligand, a TCR, variable domain (Va, Vb, Vg, Vd) of a TCR and a receptor. In some embodiments of any of the single-chain SARs described herein, the heterologous antigen-binding domain comprises a scFv.

In some embodiments of any of the single-chain SARs described herein, the heterologous antigen-binding region binds specifically to a single antigen. In some embodiments of any of the single-chain SARs described herein, the single antigen is a tumor antigen. In some embodiments of any of the single-chain SARs described herein, the tumor antigen is selected from an antigen listed in Table B.

In some embodiments of any of the single-chain SARs described herein, when going in the N-terminal to the C-terminal direction or in the C-terminal to the N-terminal direction, the single-chain SARs includes the non-naturally occurring extracellular antigen binding domain(s), the optional linker, the optional extracellular ligand-binding domain(s) of a naturally occurring receptor, the optional hinge domain, the transmembrane domain, the optional cytosolic co-stimulatory domain and the optional cytosolic primary signaling domain comprising the ITAM. In some embodiments of any of the single-chain SARs described herein, the transmembrane domain and the optional cytosolic primary signaling domain directly abut each other. In some embodiments of any of the single-chain SARs described herein, the transmembrane domain and the optional cytosolic primary signaling domain are separated by 1 to 500 amino acids (e.g., 1 to 250 amino acids, or 1 to 50 amino acids). In some embodiments of any of the single-chain SARs described herein, the optional primary signaling domain and the costimulatory domain directly abut each other. In some embodiments of any of the single-chain SARs described herein, the optional primary signaling domain and the costimulatory domain are separated by 1 to 500 amino acids (e.g., 1 to 250 amino acids, or 1 to 50 amino acids). In some embodiments of any of the single-chain SARs described herein, the costimulatory domain and the ITAM directly abut each other. In some embodiments of any of the single-chain SARs described herein, the costimulatory domain and the ITAM are separated by 1 to 500 amino acids (e.g., 1 to 250 amino acids, or 1 to 50 amino acids).

In some embodiments of any of the single-chain SARs described herein, when going in the N-terminal to the C-terminal direction or in the C-terminal to the N-terminal direction, the single-chain SAR includes the non-naturally occurring extracellular antigen binding domain(s), the optional linker, the optional extracellular ligand-binding domain(s) of a naturally occurring receptor, the optional hinge domain, the transmembrane domain, the costimulatory domain, the primary signaling domain, and the ITAM.

In some embodiments of any of the single-chain SARs described herein, when going in the N-terminal to the C-terminal direction or in the C-terminal to the N-terminal direction, the single-chain SAR includes the non-naturally occurring extracellular antigen binding domain(s), the transmembrane domain, the primary signaling domain, the ITAM, and the costimulatory domain.

In some embodiments of any of the single-chain SARs described herein, when going in the N-terminal to the C-terminal direction or in the C-terminal to the N-terminal direction, the single-chain synthetic antigen receptor includes the non-naturally occurring extracellular antigen binding domain(s), the transmembrane domain, the second intracellular signaling domain, the ITAM, and the first intracellular signaling domain.

In some embodiments of any of the single-chain SARs described herein, when going to the N-terminal to the C-terminal direction or in the C-terminal to the N-terminal direction, the single-chain synthetic antigen receptor includes the heterologous extracellular antigen binding domain(s), the transmembrane domain, the ITAM, the primary signaling domain, and the costimulatory domain.

In some embodiments of any of the single-chain SARs described herein, when going to the N-terminal to the C-terminal direction or in the C-terminal to the N-terminal direction, the single-chain synthetic antigen receptor includes the non-naturally occurring extracellular antigen binding domain(s), the transmembrane domain, the ITAM, the second intracellular signaling domain, and the first intracellular signaling domain.

In some embodiments of any of the single-chain SARs described herein, the extracellular antigen-binding domain and the transmembrane domain directly abut each other. In some embodiments of any of the single-chain SARs described herein, the extracellular antigen-binding domain and the transmembrane domain are separated by 1 to 500 amino acids (e.g., 1 to 250 amino acids, or 1 to 50 amino acids).

In some embodiments of any of the single-chain SARs described herein, the primary signaling domain is from CD3z. In some embodiments of any of the single-chain SARs described herein, the primary signaling domain is from FcRγ. In some embodiments of any of the single-chain SARs described herein, the primary signaling domain is from DAP10. In some embodiments of any of the single-chain SARs described herein, the primary signaling domain is from DAP12.

Also provided herein are nucleic acids that include a nucleotide sequence encoding any of the single-chain SARs described herein. Also provided herein are vectors that include any of the nucleic acids described herein that include a nucleotide sequence encoding any of the single-chain SARs described herein.

Also provided herein are mammalian cells that include any of the vectors described herein. In some embodiments of any of the mammalian cells described herein, the mammalian cell is a T cell, NK cell, macrophage, or an iPSC.

Also provided herein are methods of generating a SAR-expressing cell, the method comprising introducing into a mammalian cell any of the nucleic acids described herein or any of the vectors described herein. In some embodiments of any of the methods described herein, the mammalian cell is a human cell. In some embodiments of any of the methods described herein, the mammalian cell is a cell selected from the group of: a CD8⁺ T cell, a CD4⁺ T cell, naïve T cell, a memory T cell, a Treg cell, natural killer T cell, an NK cell, B cell, and a macrophage/monocyte. In some embodiments of any of the methods described herein, the mammalian cell is a cell selected from the group of: iPSC (induced pluripotent stem cell or embryonic stem cell or hematopoietic stem cell that can give rise to an immune effector cell (e.g., a T cell, NK cell or NKT cell). In some embodiment, the mammalian cell is an immortalized cell line, such as NK92, NK92MI or a derivative thereof. In some embodiments of any of the methods described herein, the mammalian cell is a mammalian cell obtained from a subject. In some embodiments of any of the methods described herein, the subject is diagnosed or identified as having a cancer. Some embodiments of any of the methods described herein further include, after the introducing step: culturing the cell in a liquid culture medium. Some embodiments of any of the methods described herein further include, before the introducing step: obtaining the mammalian cell from the subject.

Also provided herein are methods of treating a cancer in a subject that include administering a therapeutically effective amount of any of the mammalian cells described herein to the subject. Some embodiments of any of the methods described herein further include, prior to the administering step, obtaining an initial cell from the subject; and introducing any of the nucleic acids described herein or any of the vectors described herein into the initial cell, to yield the mammalian cell that is administered to the subject. Some embodiments of any of the methods described herein further include, between the introducing step and the administering step, a step of culturing the cell that is administered to the subject in a liquid culture medium. In some embodiments of any of the methods described herein, the subject is human.

Provided herein are multi-chain SARs that include at least one first polypeptide that includes: an extracellular antigen-binding domain; an optional hinge domain, a transmembrane domain; and an optional cytosolic domain.

In some embodiments of any of the multi-chain SARs described herein, the extracellular antigen-binding domain is selected from the group of: Vα, Vβ, Vγ, Vδ, vL, vH domain, a scFv, a (scFv)₂, a VHH domain, FHVH (a fully human vH domain), a single domain antibody, a non-immunoglobulin antigen binding scaffold, a VNAR domain, a ligand and a receptor. In some embodiments of any of the multi-chain SARs described herein, the extracellular antigen-binding domain comprises a scFv.

In some embodiments of any of the multi-chain SARs described herein, the at least one first polypeptide includes the extracellular antigen-binding region that binds specifically to a single antigen. In some embodiments of any of the multi-chain SARs described herein, the single antigen is a tumor antigen.

In some embodiments of any of the multi-chain SARs described herein, the SAR lacks an ITAM but recruits a signaling protein comprising a primary stimulating domain containing an ITAM. In some embodiments of any of the multi-chain SARs described herein, the SARs recruits a signaling protein selected from the group of CD3z, FcRγ, DAP10 and/DAP10.

In some embodiments of any of the multi-chain SARs described herein, when going in the N-terminal to the C-terminal direction or in the C-terminal to the N-terminal direction, the at least first polypeptide of the multi-chain SARs includes the heterologous antigen binding domain(s), the optional linker, the optional extracellular domain of a naturally occurring receptor, the optional hinge domain, the transmembrane domain, the optional cytosolic co-stimulatory domain and the optional cytosolic primary signaling domain comprising the ITAM. In some embodiments of any of the at least first polypeptide of the multi-chain SARs described herein, the transmembrane domain and the optional cytosolic primary signaling domain directly abut each other. In some embodiments of any of the at least first polypeptide of the multi-chain SARs described herein, the transmembrane domain and the optional cytosolic primary signaling domain are separated by 1 to 500 amino acids (e.g., 1 to 250 amino acids, or 1 to 50 amino acids). In some embodiments of any of the at least first polypeptide of the multi-chain SARs described herein, the optional primary signaling domain and the costimulatory domain directly abut each other. In some embodiments of any of the at least first polypeptide of the multi-chain SARs described herein, the optional primary signaling domain and the costimulatory domain are separated by 1 to 500 amino acids (e.g., 1 to 250 amino acids, or 1 to 50 amino acids). In some embodiments of any of the at least first polypeptide of the multi-chain SARs described herein, the costimulatory domain and the ITAM directly abut each other. In some embodiments of any of the at least first polypeptide of multi-chain SARs described herein, the costimulatory domain and the ITAM are separated by 1 to 500 amino acids (e.g., 1 to 250 amino acids, or 1 to 50 amino acids).

In some embodiments of any of the at least first polypeptide of the multi-chain SARs described herein, when going in the N-terminal to the C-terminal direction or in the C-terminal to the N-terminal direction, the multi-chain SAR includes the heterologous antigen binding domain(s), optional linker, optional hinge domain, the transmembrane domain, the costimulatory domain, the primary signaling domain, and the ITAM.

In some embodiments of any of the at least first polypeptide of the multi-chain SARs described herein, when going in the N-terminal to the C-terminal direction or in the C-terminal to the N-terminal direction, the at least first polypeptide of multi-chain SAR includes the heterologous antigen binding domain(s), the transmembrane domain, the primary signaling domain, the ITAM, and the costimulatory domain.

In some embodiments of any of the at least first polypeptide of the multi-chain SARs described herein, when going in the N-terminal to the C-terminal direction or in the C-terminal to the N-terminal direction, the at least first polypeptide of the multi-chain SARs includes the heterologous antigen binding domain(s), the transmembrane domain, the second intracellular signaling domain, the ITAM, and the first intracellular signaling domain.

In some embodiments of any of the at least first polypeptide of the multi-chain SARs described herein, when going to the N-terminal to the C-terminal direction or in the C-terminal to the N-terminal direction, the at least first polypeptide of the multi-chain SARs includes the extracellular antigen binding domain, the transmembrane domain, the ITAM, the primary signaling domain, and the costimulatory domain.

In some embodiments of any of the at least first polypeptide of the multi-chain SARs described herein, when going to the N-terminal to the C-terminal direction or in the C-terminal to the N-terminal direction, the first polypeptide of the multi-chain SARs includes the extracellular antigen binding domain, the transmembrane domain, the ITAM, the second intracellular signaling domain, and the first intracellular signaling domain.

In some embodiments of any of the at least first polypeptide of the multi-chain SARs described herein, the extracellular antigen-binding domain and the transmembrane domain directly abut each other. In some embodiments of any of the at least first polypeptide of the multi-chain SARs described herein, the extracellular antigen-binding domain and the transmembrane domain are separated by 1 to 500 amino acids (e.g., 1 to 250 amino acids, or 1 to 50 amino acids).

In some embodiments of any of the at least first polypeptide of the multi-chain SARs described herein, the primary signaling domain is from one or more of proteins selected from the group of CD3z, FcRγ, DAP10 or DAP12.

Other features and advantages of the invention will be apparent from the following detailed description and figures, and from the claims.

### BRIEF DESCRIPTION OF DRAWINGS

The invention is further described in the following non-limiting figures.

FIG. 1 shows schematic representation of different double chain unispecific, bispecific and multispecific SARs.

FIG. 2 shows a schematic representation of different double chain unispecific, bispecific and multispecific SARs comprising different forms of AABD (e.g., vHH, SVH, aVH, affibody, Centyrin etc.).

FIG. 3 show depictions of various formats that single-chain and double-chain CD16-SARs of the disclosure can have upon expression. These SARs are based on the entire extracellular domain of CD16 comprising both its Ig like domains (D1 and D2 domains).

FIG. 4 show depictions of various formats that CD16-SARs of the disclosure can have upon expression. These SARs are based on the partial extracellular domain of CD16. As SARs are modular in format, the CD16 modules can be substituted by different modules derived from NKp44, NKp46 etc. to generate diverse SARs.

FIG. 5 show depictions of various formats that NKp30 SARs of the disclosure can have upon expression. As SARs are modular in format, the NKp30 modules can be substituted by different modules derived from NKp44, NKp46 etc. to generate diverse SARs.

FIG. 6 shows the results of Matador cytotoxicity assay with NK92 cells expressing the indicated SAR constructs when co-cultured with the RS4; 11 GLuc target cells expressing CD19 for 2 hours.

FIG. 7 shows the results of Matador cytotoxicity assay with NK92 cells expressing the indicated SAR constructs when co-cultured with the RS4; 11 GLuc target cells for 2 hours.

FIG. 8 shows the results of Matador cytotoxicity assay with NK92 cells expressing the indicated SAR constructs when co-cultured with the L363-GLuc target cells for 2 hours.

FIG. 9 shows the results of Matador cytotoxicity assay with NK92 cells expressing the indicated SAR constructs when co-cultured with the RS4; 11 Gluc target cells for 2 hours.

FIG. 10 shows the results of Matador cytotoxicity assay with NK92 cells expressing the indicated SAR constructs when co-cultured with the RS4; 11 GLuc target cells for 2 hours.

FIG. 11 shows the results of Matador cytotoxicity assay with NK92 cells expressing the indicated SAR constructs when co-cultured with the L363-Gluc target cells for 2 hours.

FIG. 12 shows a general description of making a SAR comprising the fusion of an antigen binding domain to the extracellular domain of a Type II membrane protein such as NKG2D

FIG. 13A-C shows (A) induction of cell death by NK92, primary NK cells and primary T cells expressing a uTCR-SAR (061621-SCjJ7; SEQ ID NO: 9366) targeting NY-ESO1 peptide (SEQ ID NO: 10880) when cocultured with U266 cells (NY-ESO1⁺/HLA-A2); and (B) upregulaton of TNFα and (C) upregulaton of IFNγ by primary T cells expressing a uTCR-SAR (061621-SCjJ7; SEQ ID NO: 9366) targeting NY-ESO1 peptide when T cells are cocultured with control T2 cells or T2 cells that had been loaded with the peptide.

### DETAILED DESCRIPTION

The invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims.

Unless otherwise defined, all technical and scientific terms used herein have the same meaning as commonly understood by one of ordinary skill in the art to which this invention belongs.

As used herein the term “comprising” or “comprises” is used in reference to compositions, methods, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.).

Generally, nomenclatures used in connection with, and techniques of, cell and tissue culture, pathology, oncology, molecular biology, immunology, microbiology, genetics and protein and nucleic acid chemistry and hybridization described herein are those well-known and commonly used in the art. The methods and techniques of the disclosure are generally performed according to conventional methods well-known in the art and as described in various general and more specific references that are cited and discussed throughout the present specification unless otherwise indicated. See, e.g., Sambrook et al., Molecular Cloning: A Laboratory Manual (4th ed., Cold Spring Harbor Laboratory Press, Cold Spring Harbor, N.Y. (2013)). The nomenclatures used in connection with, and the laboratory procedures and techniques of, immunology, molecular biology, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

The term “autonomous antigen binding domain” or “AABD” as used herein refers to an antigen binding domain that can bind to an antigen autonomously, i.e., in the absence of another antigen binding domain. An exemplary AABD is a single vH domain or an autonomous vH domain (aVH), typically a single human vH domain (SVH) that can bind an antigen in the absence of a vL domain. Another exemplary AABD is a fully human vH domain (FHVH). Another exemplary AABD is a single vL domain or an autonomous vL domain, typically a single human vL domain (SVL) that can bind an antigen in the absence of a vH domain. AABD also refers to other antigen binding domains that can bind an antigen autonomously. In an embodiment, the AABD is a non-scFv antigen binding domain. An exemplary non-scFV based autonomous antigen binding domain includes but is not limited to a vHH domain, a humanized vHH domain, a single variable domain-TCR (svd-TCR), and non-immunoglobulin antigen binding scaffold such as a DARPIN, an affibody, a ZIP domain (e.g., RZIP, EZIP, E4, R4 etc.), an affilin, an adnectin, an affitin, an obody, a repebody, a fynomer, an alphabody, an avimer, an atrimer, a centyrin, a pronectin, an anticalin, a kunitz domain, an Armadillo repeat protein or a fragment thereof. Additional examples of non-scFV based autonomous antigen binding domains include the ligand binding domain of a receptor (e.g., CD16-V158A, NKG2D) or a fragment thereof, the receptor binding domain of a ligand (e.g., APRIL, Thrombopoietin etc.) or a fragment thereof, an adaptor (e.g., RZIP, EZIP, E4, K4, NKG2D-YA, NKG2D-AF etc.) or a fragment thereof, an adatptor binding protein (e.g. ULBP2R, ULBP2-S3 etc.) or a fragment thereof, an epitope or a tag (e.g., Streptag, FLAG tag etc.), an autoantigen or a fragment thereof and the like.

The disclosure described the use of AABD, such as human VH (or vH) domains, such as multiple human VH domains, as building blocks to make unispecific, bispecific and multispecific SARs. In an embodiment, the disclosure describes the use of AABD, such as human VH domains, such as multiple human VH domains, as building blocks to make unispecific, bispecific and multispecific novel SARs.

The term “about” when referring to a measurable value such as an amount, a temporal duration, and the like, is meant to encompass variations of ±20% or in some instances ±10%, or in some instances ±5%, or in some instances ±1%, or in some instances ±0.1% from the specified value, as such variations are appropriate to perform the disclosed methods or describe the compositions herein. Moreover, any value or range (e.g., less than 20 or similar terminology) explicitly includes any integer between such values or up to the value. Thus, for example, “one to five mutations” explicitly includes 1, 2, 3, 4, and/or 5 mutations.

The term “ABR” or “Antigen Binding Receptor” as described herein refers to any receptor that has an antigen binding domain. The antigen binding domain of an ABR may comprise of a scFv, a vL, vH, VHH, antibody, antibody fragment (e.g., Fab), antibody like moiety, Va, VD, svd-TCR, cytokine, receptor etc. In one embodiment, an ABR has a transmembrane or membrane anchoring domain that allows it to be expressed on the cell surface. Exemplary ABR include a 1^{st} generation CAR, a 2^{nd} generation CAR, a TFP, SIR, STAR, zSIR, cTCR, TCR, Ab-TCR, a TRI-TAC or TAC etc. Synthetic antigen receptors (SARs), as described herein, are also examples of ABR.

The term “Ab-TCR” or “AbTCR” refers to a next generation CAR platform as described in WO 2017/070608 A1 which is incorporated herein by reference. In an embodiment, an Ab-TCR comprises an antibody moiety that specifically binds to a target antigen fused to a TCR module capable of recruiting at least one TCR signaling module. Exemplary TCR modules that can be used in the construction of Ab-TCR are provided in SEQ ID NO:6009-6014 (Table 6) and in WO 2017/070608 A1 which is incorporated herein by reference.

The term “accessory module” refers to any one or more of PDL1, PDL2, CD80, CD86, crmA, p35, hNEMO-K277A (or NEMO-K277A), hNEMO-K277A-delta-V249-K555, mNEMO-K270A, K13-opt, IKK2-S177E-S181E (or IKK2-SS/EE), IKK1-S176E-S180E (or IKK1-SS/EE), MyD88-L265P, TCL-1a, MTCP-1, CMV-141, 41BBL, CD40L, vFLIP-K13, MC159, cFLIP-L/MRITα, cFLIP-p22, HTLV1 Tax, HTLV2 Tax, HTLV2 Tax-RS mutant, FKBPx2-K13, FKBPx2-HTLV2-Tax, FKBPx2-HTLV2-Tax-RS, IL6R-304-vHH-Alb8-vHH, IL12f, PD1-4H1 scFV, PD1-5C4 scFV, PD1-4H1-Alb8-vHH, PD1-5C4-Alb8-vHH, CTLA4-Ipilimumab-scFv, CTLA4-Ipilimumab-Alb8-vHH, IL6-19A-scFV, IL6-19A-scFV-Alb8-vHH, sHVEM, sHVEM-Alb8-vHH, hTERT, Fx06, shRNA targeting Brd4, IgSP-[hTRAC-opt2], IgSP-[hTRBC-opt2], a multi-purpose switch (e.g., IL2-tBCMA, IL15-tBCMA, IL2-RQR, IL15-RQR etc.), NKG2C, CD94, DAP10, DAP12, CD3ε, CD3γ, CD3δ, CD3ƒ, FcRγ, and combination thereof that is expressed in an immune cell (e.g., NK cell or T cell, e.g., SAR-NK cell, SAR-T cell or TCR-T cell) to decrease, regulate or modify the activity of the immune cell. In an embodiment, an accessory module is a therapeutic control (e.g., icapase 9). In some embodiments, the accessory module is co-expressed with an immune receptor such as a SAR or a TCR to increase, decrease, regulate or modify the expression or activity of a SAR or a TCR or a SAR-expressing or a TCR-expressing cell. The accessory module can be co-expressed with a SAR or a TCR using a single vector or using two or more different vectors. In some embodiments, the accessory module is expressed in an antigen presenting cell, e.g., a dendritic cell.

As used herein “affinity” is meant to describe a measure of binding strength. Affinity generally refers to the “ability” of the binding agent to bind its target. There are numerous ways used in the art to measure “affinity”. For example, methods for calculating the affinity of an antibody for an antigen are known in the art, including use of binding experiments to calculate affinity. As used herein, the term “specific binding” means the contact between an antibody and an antigen with a binding affinity of at least 10⁻⁶ M. In certain aspects, antibodies bind with affinities of at least about 10⁻⁷M, and typically 10⁻⁸ M, 10⁻⁹ M, 10⁻¹⁰ M, 10⁻¹¹ M, or 10⁻¹² M.

The term “antibody,” as used herein, refers to a protein, or polypeptide sequence derived from an immunoglobulin molecule which specifically binds with an antigen. Antibodies can be monoclonal, or polyclonal, multiple or single chain, or intact immunoglobulins, and may be derived from natural sources or from recombinant sources. The antibody may be ‘humanized’, ‘chimeric’, fully human or non-human. An antibody may have a single domain (e.g., a single vH domain).

The term “antibody fragment” refers to at least one portion of an antibody, that retains the ability to specifically interact with (e.g., by binding, steric hindrance, stabilizing/destabilizing, spatial distribution) an epitope of an antigen. Examples of antibody fragments include, but are not limited to, Fab, Fab′, F(ab′h, Fv fragments, scFv antibody fragments, disulfide-linked Fvs (sdFv), a Fd fragment consisting of the VH and CHl domains, linear antibodies, single domain antibodies (sdAb) such as either vL or vH, camelid vHH domains, multi-specific antibodies formed from antibody fragments such as a bivalent fragment comprising two Fab fragments linked by a disulfide bridge at the hinge region, and an isolated CDR or other epitope binding fragments of an antibody. An antigen binding fragment can also be incorporated into single domain antibodies, maxibodies, minibodies, nanobodies, intrabodies, diabodies, triabodies, tetrabodies, v-NAR and bis-scFv (see, e.g., Hollinger and Hudson, Nature Biotechnology 23:1126-1136, 2005). Antigen binding fragments can also be grafted into scaffolds based on polypeptides such as a fibronectin type III (Fn3) (see U.S. Pat. No. 6,703,199, which describes fibronectin polypeptide mini-bodies).

The term “antibody heavy chain,” refers to the larger of the two types of polypeptide chains present in antibody molecules in their naturally occurring conformations, and which normally determines the class to which the antibody belongs.

The term “antibody light chain,” refers to the smaller of the two types of polypeptide chains present in antibody molecules in their naturally occurring conformations. Kappa (κ) and lambda (λ) light chains refer to the two major antibody light chain isotypes.

“Anticancer agent” refers to agents that inhibit aberrant cellular division and growth, inhibit migration of neoplastic cells, inhibit invasiveness or prevent cancer growth and metastasis. The term includes chemotherapeutic agents, biological agent (e.g., siRNA, viral vectors such as engineered MLV, adenoviruses, herpes virus that deliver cytotoxic genes), antibodies and the like.

The term “anticancer effect” or “anti-tumor effect” refers to a biological effect which can be manifested by various means, including but not limited to, a decrease in tumor volume, a decrease in the number of cancer cells, a decrease in the number of metastases, an increase in life expectancy, decrease in cancer cell proliferation, decrease in cancer cell survival, or amelioration of various physiological symptoms associated with the cancerous condition. An “anticancer effect” can also be manifested by the ability of the SARs to prevent the occurrence of cancer in the first place.

The term “antigen” or “Ag” refers to a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. As used herein, the term “antigen” refers generally to a binding partner specifically recognized by an antigen-binding domain described herein. Non-limiting examples of antigen or antigens that can be specifically bound by any of the antigen-binding domains are described in Table B.

The term “antigen presenting cell” or “APC” refers to an immune system cell such as an accessory cell (e.g., a B-cell, a dendritic cell, and the like) that displays a foreign antigen complexed with major histocompatibility complexes (MHC's) on its surface.

The term “anti-infection effect” refers to a biological effect that can be manifested by various means, including but not limited to, e.g., decrease in the titer of the infectious agent, a decrease in colony counts of the infectious agent, amelioration of various physiological symptoms associated with the infectious condition.

An “antigen binding domain” or “antigen binding module” or “antigen binding segment” or “antigen specific domain” (ASD) refers to a polypeptide or peptide that due to its primary, secondary or tertiary sequence, post-translational modifications and/or charge binds to an antigen with a high degree of specificity. An ASD can bind to a target with affinity higher than a non-specific domain. The antigen binding domain may be derived from different sources, for example, an antibody (full length heavy chain, Fab fragments, single chain Fv (scFv) fragments, divalent single chain antibodies or diabodies), a non-immunoglobulin binding protein, a ligand or a receptor. In some embodiments, almost any molecule that binds a given cognate or antigen with high affinity can be used as an ASD, as will be appreciated by those of skill in the art. In some embodiments, the antigen binding domain comprises T cell receptors (TCRs) or portions thereof. In exemplary embodiments, the target antigens and SEQ ID Nos of various antigen binding domains are set forth herein in Tables 3-7. In exemplary embodiments, the target antigen and SEQ ID NOs of vL, vH, scFVs, and their CDR regions are set forth herein in Tables 6A-C of patent application PCT/US18/53247 and in Tables 3-4 of patent application PCT/US19/035096, which are incorporated in their entirety by reference herein.

The term “Association constant (Ka)” is defined as the equilibrium constant of the association of a receptor and ligand.

“Autoantibody” refers to an antibody that is produced by a B-cell specific for an autoantigen.

The term “autoantigen” refers to an endogenous antigen that stimulates production of an autoimmune response, such as production of autoantibodies. Examples of autoantigens include, but are not limited to, desmoglein 1, desmoglein 3, and fragments thereof.

Avidity” refers to the strength of the interaction between a binding agent and its target (e.g., the strength of the interaction between an antibody and its antigen target, a receptor and its cognate and the like). Antibody activity in functional assays (e.g., flow cytometry assay or Malibu-Glo assay) is also reflective of antibody affinity.

As used herein, the term “backbone” or “architecture” refers to the configuration of the different components (e.g., antigen binding domains, hinge domains, transmembrane domains, signaling domains) that comprise different SAR and any accessory module which is generally optional. In one embodiment, the SAR and the accessory module are encoded by a single nucleic acid molecule. In another embodiment, the SAR is encoded by the first nucleic acid molecule and the accessory module is encoded by a second nucleic acid molecule. In some embodiments, the accessory module is encoded by more than one nucleic acid molecule, depending on the number of components in the accessory modules. The two or more components of the SAR and the accessory modules may be separated by a cleavable linker such as a 2A ribosomal skip sequence (e.g., P2A, T2A, F2A etc.). The two or more components of the SAR and accessory modules may be separated by an internal ribosomal entry sequence (IRES). An exemplary IRES is derived from KSHV. The expression of nucleic acids encoding two or more components of the SAR and accessory modules may be driven by separate promoters. Exemplary promoters include EFlu, EFS, EFS2, CMV, RSV, mutRSV, MNDU3, Hsp70 and Hsp90.

Table A1: Conventional CAR architectures. First generation conventional CARs (Conventional CAR I) have an intracellular signaling (ISD) domain (e.g., CD3z) and no costimulatory domain. The TCR fusion proteins (TFP) are another example of conventional CAR 1. Second generation conventional CARs (Conventional CAR 2 or CAR II) have one costimulatory domain (e.g., 41BB or CD28) and an intracellular signaling (ISD) domain (e.g., CD3z). Third generation conventional CARs (Conventional CAR 3 or CAR III) have two costimulatory domains (e.g., 41BB and CD28) and an intracellular signaling (ISD) domain (e.g., CD3z). Ab-TCRs are duel chain receptors incorporating a vL-linker-TCR domain (TCRD and a vH-linker-TCR domain (TCRD) and have been described in PCT/US2016/058305. cTCRs are single chain, one-and-half, or double chain receptors consisting of antigen binding domain derived from a vL and vH fragment that are fused to one or more TCR constant chain (TCR-C) and result in activation of T cell signaling. The TCR constant chains of cTCRs are encoded by wild-type nucleic acid sequences and corresponding wild-type amino acid sequences. Different configurations of cTCR are described in PCT/US2017/064379 or WO 2018/102795 A1. Synthetic immune receptors are next generation CARs and are described in PCT/US2017/064379 or WO 2018/102795 A1. SIRs are single chain, one-and-half, or double chain receptors. In one embodiment, the antigen binding domain of SIR are derived from a vL and vH fragment that are fused to one or more TCR constant chain (TCR-C) and result in activation of T cell signaling. In some embodiments, the TCR constant chains of SIR are encoded by codon-optimized nucleic acid sequences and comprise one or more mutations that enhance their expression and chain-pairing. zSIRs are double chain receptors comprising that comprise antigen binding domains (e.g., vL, vH etc.) that are operationally linked to two CD3z chains or fragments thereof with optional linkers and are described in PCT/US2019/035096.

TABLE A1
Exemplary CONVENTIONAL CAR Architectures

| | | | | | | | |
|---|---|---|---|---|---|---|---|
| 1 | CAR 1 or CAR I | ASD | HR | TMD | ISD | | |
| | (including TFP) | | | | | | |
| 2 | CAR 2 (CAR II) | ASD | HR | TMD | CSD | ISD | |
| 3 | CAR 3 (CAR III) | ASD | HR | TMD | CSD-I | CSD-II | ISD |
| 4 | Ab-TCR | vL-cL | TCRD(1) | 2A | vH-CH1 | TCRD (II) | |
| 5 | Double Chain | vL | TCR-C(1) | 2A | vH | TCR-C (II) | |
| | cTCR/SIR-1 | | | | | | |
| 6 | Double Chain | vL- | CD3z | 2A | vH- | CD3z | |
| | zSIR | linker | | | linker | | |
| 6 | One & Half Chain | | TCR-C(1) | 2A | ASD | TCR-C (II) | |
| | cTCR/SIR-3 | | | | | | |

TABLES A1-1 to A1-19 provide exemplary architectures of unispecific, bispecific and multispecific SARs of this disclosure. The abbreviations used are: SP (signal peptide); AADB (autonomous antigen binding domain); L (optional linker); LL (Long linker), (AABD-L)n (n copies of AABD with optional linker where n=0, 1, 2, 3, 4 or more), AABD1-4 (different AABD targeting one or more antigens), V1 (vL, vH, Va, Vb, Vg or Vd chains), Ig (Ig linker), TCR-Ig (Ig linker domain derived from TCR chains), ConP (connecting peptide), TM (transmembrane domain), Cβ (cytosolic domain), IC (intracellular domain), Ca (Constant chain of TCRα), Cb (constant chain of TCRβ), Cg (constant chain of TCRγ), Cd (constant chain of TCRδ), scFv (single chain fragment variable), scTFv (single chain fragment comprising two variable fragments of a TCR, e.g, Va and Vb), dCa/dCb/dCg/dCd (N-terminally deleted constant chain of TCRα, β, γ or δ lacking their Ig linker domain), TCR-ConP (connecting peptide of TCRα, β, γ or δ constant chain), Ca-ConP (connecting peptide of TCRα constant chain), IgCL (Ig linker from immunoglobulin light chain), IgCH1 (Ig liker from immunoglobulin heavy chain), CD3εγδ ECD (extracellular domain of CD3ε, γ or δ chains), CSD (costimulatory domain), 4-1BB or BB (costimulatory domain of 4-1BB), CD28 or 28 (costimulatory domain of CD28), CD3z or zd or z (activation domain of CD3z). NKIp30-Ig (Immunoglobulin like domain of Nkp30), NKIp44-Ig (Immunoglobulin like domain of Nkp44), NKIp46-Ig1-Ig2 (Immunoglobulin like domain 1 and 2 of Nkp46), CD16-D1 (Domain 1 of CD16), CD16-D2 (Domain 2 of CD16), scTCR (Single chain TCR), Extracellular domain (ECD), activation domain (AD). Va, Vb, Vg, Vd (variable domains of TCRα, β, γ and δ), FCRG (FcRγ); Hinge domain (Hn).

TABLE A1-1
EXEMPLARY CD16-BASED SINGLE CHAIN SARS

| SAR | | | | | | | | | | |
|---|---|---|---|---|---|---|---|---|---|---|
| Class | | | | | | | | | | |
| 1 | SP | L | (AABD- | scFv | L | LL | TM | AD | | |
| | | | L)n | | | | | | | |
| 2 | SP | L | (AABD- | scFv | L | Hinge | TM | AD | | |
| | | | L)n | | | | | | | |
| 3 | SP | L | (AABD- | (scFv- | L | CD16- | CD16- | CD16- | CD16- | CD16- |
| | | | L)n | L)n | | D1 | D2 | Hinge | TM | CP |
| 4 | SP | L | (AABD- | (scFv- | L | CD8- | CD16- | CD16- | CD16- | |
| | | | L)n | L)n | | Hinge | Hinge | TM | CP | |
| 5 | SP | L | (AABD- | (scFv- | L | CD28- | CD16- | CD16- | CD16- | |
| | | | L)n | L)n | | Hinge | Hinge | TM | CP | |
| 6 | SP | L | (AABD- | (scFv- | L | CD8- | CD16- | CD16- | | |
| | | | L)n | L)n | | Hinge | TM | CP | | |
| 7 | SP | L | (AABD- | (scFv- | L | CD28- | CD16- | CD16- | | |
| | | | L)n | L)n | | Hinge | TM | CP | | |
| 8 | SP | | | (scFv- | L | CD16- | CD16- | CD16- | CD16- | CD16- |
| | | | | L)n | | D1 | D2 | Hinge | TM | CP |
| 9 | SP | | | (scFv- | L | CD8- | CD16- | CD16- | CD16- | |
| | | | | L)n | | Hinge | Hinge | TM | CP | |
| 10 | SP | | | (scFv- | L | CD28- | CD16- | CD16- | CD16- | |
| | | | | L)n | | Hinge | Hinge | TM | CP | |
| 11 | SP | | | (scFv- | L | CD8- | CD16- | CD16- | | |
| | | | | L)n | | Hinge | TM | CP | | |
| 12 | SP | | | (scFv- | L | CD28- | CD16- | CD16- | | |
| | | | | L)n | | Hinge | TM | CP | | |
| 13 | SP | L | (AABD- | (scTCR- | L | CD16- | CD16- | CD16- | CD16- | CD16- |
| | | | L)n | L)n | | D1 | D2 | Hinge | TM | CP |
| 14 | SP | L | (AABD- | (scTCR- | L | CD8- | CD16- | CD16- | CD16- | |
| | | | L)n | L)n | | Hinge | Hinge | TM | CP | |
| 15 | SP | L | (AABD- | (scTCR- | L | CD28- | CD16- | CD16- | CD16- | |
| | | | L)n | L)n | | Hinge | Hinge | TM | CP | |
| 16 | SP | L | (AABD- | (scTCR- | L | CD8- | CD16- | CD16- | | |
| | | | L)n | L)n | | Hinge | TM | CP | | |
| 17 | SP | L | (AABD- | (scTCR- | L | CD28- | CD16- | CD16- | | |
| | | | L)n | L)n | | Hinge | TM | CP | | |
| 18 | SP | L | | scTCR | L | CD16- | CD16- | CD16- | CD16- | CD16- |
| | | | | | | D1 | D2 | Hinge | TM | CP |
| 19 | SP | L | | scTCR | L | CD8- | CD16- | CD16- | CD16- | |
| | | | | | | Hinge | Hinge | TM | CP | |
| 20 | SP | L | | scTCR | L | CD28- | CD16- | CD16- | CD16- | |
| | | | | | | Hinge | Hinge | TM | CP | |
| 21 | SP | L | | scTCR | L | CD8- | CD16- | CD16- | | |
| | | | | | | Hinge | TM | CP | | |
| 22 | SP | L | | scTCR | L | CD28- | CD16- | CD16- | | |
| | | | | | | Hinge | TM | CP | | |
| 23 | SP | L | (AABD- | | L | CD16- | CD16- | CD16- | CD16- | CD16- |
| | | | L)n | | | D1 | D2 | Hinge | TM | CP |
| 24 | SP | L | (AABD- | | L | CD8- | CD16- | CD16- | CD16- | |
| | | | L)n | | | Hinge | Hinge | TM | CP | |
| 25 | SP | L | (AABD- | | L | CD28- | CD16- | CD16- | CD16- | |
| | | | L)n | | | Hinge | Hinge | TM | CP | |
| 26 | SP | L | (AABD- | | L | CD8- | CD16- | CD16- | | |
| | | | L)n | | | Hinge | TM | CP | | |

| | | | | | | | |
|---|---|---|---|---|---|---|---|
| 27 | SP | L | (AABD-L)n | L | CD28- | CD16- | CD16- |

| | | |
|---|---|---|
| Hinge | TM | CP |

TABLE A1-2
EXEMPLARY Nkp30-BASED SINGLE CHAIN SARS

| SAR | | | | | | | | | |
|---|---|---|---|---|---|---|---|---|---|
| Class | | | | | | | | | |
| 1 | SP | L | (AABD- | scFv | L | LL | TM | AD | |
| | | | L)n | | | | | | |
| 2 | SP | L | (AABD- | scFv | L | Hinge | TM | AD | |
| | | | L)n | | | | | | |
| 3 | SP | L | (AABD- | (scFv- | L | Nkp30- | Nkp30- | Nkp30- | Nkp30- |
| | | | L)n | L)n | | Ig | Hinge | TM | CP |
| 4 | SP | L | (AABD- | (scFv- | L | CD8- | Nkp30- | Nkp30- | Nkp30- |
| | | | L)n | L)n | | Hinge | Hinge | TM | CP |
| 5 | SP | L | (AABD- | (scFv- | L | CD28- | Nkp30- | Nkp30- | Nkp30- |
| | | | L)n | L)n | | Hinge | Hinge | TM | CP |
| 6 | SP | L | (AABD- | (scFv- | L | CD8- | Nkp30- | Nkp30- | |
| | | | L)n | L)n | | Hinge | TM | CP | |
| 7 | SP | L | (AABD- | (scFv- | L | CD28- | Nkp30- | Nkp30- | |
| | | | L)n | L)n | | Hinge | TM | CP | |
| 8 | SP | | | (scFv- | L | Nkp30- | Nkp30- | Nkp30- | Nkp30- |
| | | | | L)n | | Ig | Hinge | TM | CP |
| 9 | SP | | | (scFv- | L | CD8- | Nkp30- | Nkp30- | Nkp30- |
| | | | | L)n | | Hinge | Hinge | TM | CP |
| 10 | SP | | | (scFv- | L | CD28- | Nkp30- | Nkp30- | Nkp30- |
| | | | | L)n | | Hinge | Hinge | TM | CP |
| 11 | SP | | | (scFv- | L | CD8- | Nkp30- | Nkp30- | |
| | | | | L)n | | Hinge | TM | CP | |
| 12 | SP | | | (scFv- | L | CD28- | Nkp30- | Nkp30- | |
| | | | | L)n | | Hinge | TM | CP | |
| 13 | SP | L | (AABD- | (scTCR- | L | Nkp30- | Nkp30- | Nkp30- | Nkp30- |
| | | | L)n | L)n | | Ig | Hinge | TM | CP |
| 14 | SP | L | (AABD- | (scTCR- | L | CD8- | Nkp30- | Nkp30- | Nkp30- |
| | | | L)n | L)n | | Hinge | Hinge | TM | CP |
| 15 | SP | L | (AABD- | (scTCR- | L | CD28- | Nkp30- | Nkp30- | Nkp30- |
| | | | L)n | L)n | | Hinge | Hinge | TM | CP |
| 16 | SP | L | (AABD- | (scTCR- | L | CD8- | Nkp30- | Nkp30- | |
| | | | L)n | L)n | | Hinge | TM | CP | |
| 17 | SP | L | (AABD- | (scTCR- | L | CD28- | Nkp30- | Nkp30- | |
| | | | L)n | L)n | | Hinge | TM | CP | |
| 18 | SP | L | | scTCR | L | Nkp30- | Nkp30- | Nkp30- | Nkp30- |
| | | | | | | Ig | Hinge | TM | CP |
| 19 | SP | L | | scTCR | L | CD8- | Nkp30- | Nkp30- | Nkp30- |
| | | | | | | Hinge | Hinge | TM | CP |
| 20 | SP | L | | scTCR | L | CD28- | Nkp30- | Nkp30- | Nkp30- |
| | | | | | | Hinge | Hinge | TM | CP |
| 21 | SP | L | | scTCR | L | CD8- | Nkp30- | Nkp30- | |
| | | | | | | Hinge | TM | CP | |
| 22 | SP | L | | scTCR | L | CD28- | Nkp30- | Nkp30- | |
| | | | | | | Hinge | TM | CP | |
| 23 | SP | L | (AABD- | | L | Nkp30- | Nkp30- | Nkp30- | Nkp30- |
| | | | L)n | | | Ig | Hinge | TM | CP |
| 24 | SP | L | (AABD- | | L | CD8- | Nkp30- | Nkp30- | Nkp30- |
| | | | L)n | | | Hinge | Hinge | TM | CP |
| 25 | SP | L | (AABD- | | L | CD28- | Nkp30- | Nkp30- | Nkp30- |
| | | | L)n | | | Hinge | Hinge | TM | CP |
| 26 | SP | L | (AABD- | | L | CD8- | Nkp30- | Nkp30- | |
| | | | L)n | | | Hinge | TM | CP | |
| 27 | SP | L | (AABD- | | L | CD28- | Nkp30- | Nkp30- | |
| | | | L)n | | | Hinge | TM | CP | |

TABLE A1-3
EXEMPLARY Nkp44-BASED SINGLE CHAIN SARS

| SAR | | | | | | | | | |
|---|---|---|---|---|---|---|---|---|---|
| Class | | | | | | | | | |
| 1 | SP | L | (AABD- | scFv | L | LL | TM | AD | |
| | | | L)n | | | | | | |
| 2 | SP | L | (AABD- | scFv | L | Hinge | TM | AD | |
| | | | L)n | | | | | | |
| 3 | SP | L | (AABD- | (scFv- | L | Nkp44- | Nkp44- | Nkp44- | Nkp44- |
| | | | L)n | L)n | | Ig | Hinge | TM | CP |
| 4 | SP | L | (AABD- | (scFv- | L | CD8- | Nkp44- | Nkp44- | Nkp44- |
| | | | L)n | L)n | | Hinge | Hinge | TM | CP |
| 5 | SP | L | (AABD- | (scFv- | L | CD28- | Nkp44- | Nkp44- | Nkp44- |
| | | | L)n | L)n | | Hinge | Hinge | TM | CP |
| 6 | SP | L | (AABD- | (scFv- | L | CD8- | Nkp44- | Nkp44- | |
| | | | L)n | L)n | | Hinge | TM | CP | |
| 7 | SP | L | (AABD- | (scFv- | L | CD28- | Nkp44- | Nkp44- | |
| | | | L)n | L)n | | Hinge | TM | CP | |
| 8 | SP | | | (scFv- | L | Nkp44- | Nkp44- | Nkp44- | Nkp44- |
| | | | | L)n | | Ig | Hinge | TM | CP |
| 9 | SP | | | (scFv- | L | CD8- | Nkp44- | Nkp44- | Nkp44- |
| | | | | L)n | | Hinge | Hinge | TM | CP |
| 10 | SP | | | (scFv- | L | CD28- | Nkp44- | Nkp44- | Nkp44- |
| | | | | L)n | | Hinge | Hinge | TM | CP |
| 11 | SP | | | (scFv- | L | CD8- | Nkp44- | Nkp44- | |
| | | | | L)n | | Hinge | TM | CP | |
| 12 | SP | | | (scFv- | L | CD28- | Nkp44- | Nkp44- | |
| | | | | L)n | | Hinge | TM | CP | |
| 13 | SP | L | (AABD- | (scTCR- | L | Nkp44- | Nkp44- | Nkp44- | Nkp44- |
| | | | L)n | L)n | | Ig | Hinge | TM | CP |
| 14 | SP | L | (AABD- | (scTCR- | L | CD8- | Nkp44- | Nkp44- | Nkp44- |
| | | | L)n | L)n | | Hinge | Hinge | TM | CP |
| 15 | SP | L | (AABD- | (scTCR- | L | CD28- | Nkp44- | Nkp44- | Nkp44- |
| | | | L)n | L)n | | Hinge | Hinge | TM | CP |
| 16 | SP | L | (AABD- | (scTCR- | L | CD8- | Nkp44- | Nkp44- | |
| | | | L)n | L)n | | Hinge | TM | CP | |
| 17 | SP | L | (AABD- | (scTCR- | L | CD28- | Nkp44- | Nkp44- | |
| | | | L)n | L)n | | Hinge | TM | CP | |
| 18 | SP | L | | scTCR | L | Nkp44- | Nkp44- | Nkp44- | Nkp44- |
| | | | | | | Ig | Hinge | TM | CP |
| 19 | SP | L | | scTCR | L | CD8- | Nkp44- | Nkp44- | Nkp44- |
| | | | | | | Hinge | Hinge | TM | CP |
| 20 | SP | L | | scTCR | L | CD28- | Nkp44- | Nkp44- | Nkp44- |
| | | | | | | Hinge | Hinge | TM | CP |
| 21 | SP | L | | scTCR | L | CD8- | Nkp44- | Nkp44- | |
| | | | | | | Hinge | TM | CP | |
| 22 | SP | L | | scTCR | L | CD28- | Nkp44- | Nkp44- | |
| | | | | | | Hinge | TM | CP | |
| 23 | SP | L | (AABD- | | L | Nkp44- | Nkp44- | Nkp44- | Nkp44- |
| | | | L)n | | | Ig | Hinge | TM | CP |
| 24 | SP | L | (AABD- | | L | CD8- | Nkp44- | Nkp44- | Nkp44- |
| | | | L)n | | | Hinge | Hinge | TM | CP |
| 25 | SP | L | (AABD- | | L | CD28- | Nkp44- | Nkp44- | Nkp44- |
| | | | L)n | | | Hinge | Hinge | TM | CP |
| 26 | SP | L | (AABD- | | L | CD8- | Nkp44- | Nkp44- | |
| | | | L)n | | | Hinge | TM | CP | |
| 27 | SP | L | (AABD- | | L | CD28- | Nkp44- | Nkp44- | |
| | | | L)n | | | Hinge | TM | CP | |

TABLE A1-4
EXEMPLARY Nkp46-BASED SINGLE CHAIN SARS

| SAR | | | | | | | | | | |
|---|---|---|---|---|---|---|---|---|---|---|
| Class | | | | | | | | | | |
| 1 | SP | L | (AABD- | scFv | L | LL | TM | AD | | |
| | | | L)n | | | | | | | |
| 2 | SP | L | (AABD- | scFv | L | Hinge | TM | AD | | |
| | | | L)n | | | | | | | |
| 3 | SP | L | (AABD- | (scFv- | L | Nkp46- | Nkp46- | Nkp46- | Nkp46- | Nkp46- |
| | | | L)n | L)n | | Ig1 | Ig2 | Hinge | TM | CP |
| 4 | SP | L | (AABD- | (scFv- | L | CD8- | Nkp46- | Nkp46- | Nkp46- | |
| | | | L)n | L)n | | Hinge | Hinge | TM | CP | |
| 5 | SP | L | (AABD- | (scFv- | L | CD28- | Nkp46- | Nkp46- | Nkp46- | |
| | | | L)n | L)n | | Hinge | Hinge | TM | CP | |
| 6 | SP | L | (AABD- | (scFv- | L | CD8- | Nkp46- | Nkp46- | | |
| | | | L)n | L)n | | Hinge | TM | CP | | |
| 7 | SP | L | (AABD- | (scFv- | L | CD28- | Nkp46- | Nkp46- | | |
| | | | L)n | L)n | | Hinge | TM | CP | | |
| 8 | SP | | | (scFv- | L | Nkp46- | Nkp46- | Nkp46- | Nkp4 | Nkp4 |
| | | | | L)n | | Ig1 | Ig2 | Hinge | 6-TM | 6-CP |
| 9 | SP | | | (scFv- | L | CD8- | Nkp46- | Nkp46- | Nkp46- | |
| | | | | L)n | | Hinge | Hinge | TM | CP | |
| 10 | SP | | | (scFv- | L | CD28- | Nkp46- | Nkp46- | Nkp46- | |
| | | | | L)n | | Hinge | Hinge | TM | CP | |
| 11 | SP | | | (scFv- | L | CD8- | Nkp46- | Nkp46- | | |
| | | | | L)n | | Hinge | TM | CP | | |
| 12 | SP | | | (scFv- | L | CD28- | Nkp46- | Nkp46- | | |
| | | | | L)n | | Hinge | TM | CP | | |
| 13 | SP | L | (AABD- | (scTCR- | L | Nkp46- | Nkp46- | Nkp46- | Nkp46- | Nkp46- |
| | | | L)n | L)n | | Ig1 | Ig2 | Hinge | TM | CP |
| 14 | SP | L | (AABD- | (scTCR- | L | CD8- | Nkp46- | Nkp46- | Nkp46- | |
| | | | L)n | L)n | | Hinge | Hinge | TM | CP | |
| 15 | SP | L | (AABD- | (scTCR- | L | CD28- | Nkp46- | Nkp46- | Nkp46- | |
| | | | L)n | L)n | | Hinge | Hinge | TM | CP | |
| 16 | SP | L | (AABD- | (scTCR- | L | CD8- | Nkp46- | Nkp46- | | |
| | | | L)n | L)n | | Hinge | TM | CP | | |
| 17 | SP | L | (AABD- | (scTCR- | L | CD28- | Nkp46- | Nkp46- | | |
| | | | L)n | L)n | | Hinge | TM | CP | | |
| 18 | SP | L | | scTCR | L | Nkp46- | Nkp46- | Nkp46- | Nkp46- | Nkp46- |
| | | | | | | Ig1 | Ig2 | Hinge | TM | CP |
| 19 | SP | L | | scTCR | L | CD8- | Nkp46- | Nkp46- | Nkp46- | |
| | | | | | | Hinge | Hinge | TM | CP | |
| 20 | SP | L | | scTCR | L | C28- | Nkp46- | Nkp46- | Nkp46- | |
| | | | | | | Hinge | Hinge | TM | CP | |
| 21 | SP | L | | scTCR | L | CD8- | Nkp46- | Nkp46- | | |
| | | | | | | Hinge | TM | CP | | |
| 22 | SP | L | | scTCR | L | CD28- | Nkp46- | Nkp46- | | |
| | | | | | | Hinge | TM | CP | | |
| 23 | SP | L | (AABD- | | L | Nkp46- | Nkp46- | Nkp46- | Nkp46- | Nkp46- |
| | | | L)n | | | Ig1 | Ig2 | Hinge | TM | CP |
| 24 | SP | L | (AABD- | | L | CD8- | Nkp46- | Nkp46- | Nkp46- | |
| | | | L)n | | | Hinge | Hinge | TM | CP | |
| 25 | SP | L | (AABD- | | L | CD28- | Nkp46- | Nkp46- | Nkp46- | |
| | | | L)n | | | Hinge | Hinge | TM | CP | |
| 26 | SP | L | (AABD- | | L | CD8- | Nkp46- | Nkp46- | | |
| | | | L)n | | | Hinge | TM | CP | | |
| 27 | SP | L | (AABD- | | L | CD28- | Nkp46- | Nkp46- | | |
| | | | L)n | | | Hinge | TM | CP | | |

TABLE A1-5
EXEMPLARY NKG2D-BASED SINGLE CHAIN SARS

| SAR Class | | | | | |
|---|---|---|---|---|---|
| 1 | NKG2D-CP | NKG2D-TM | NKG2D-ECD | L | (AABD) |
| 2 | NKG2D-CP | NKG2D-TM | NKG2D-ECD | L | (AABD-L)n |
| 3 | NKG2D-CP | NKG2D-TM | NKG2D-ECD | L | scFv |
| 4 | NKG2D-CP | NKG2D-TM | NKG2D-ECD | L | (scFv-L)n |

TABLE: A1-6
EXEMPLARY SARS

| SAR | | | | | | | | |
|---|---|---|---|---|---|---|---|---|
| Class | | | | | | | | |
| 1 | SP | L | (AABD- | L | Ig (Ig | TCR- | TM | IC |
| | | | L)n | | linker) | ConP | | |
| | SP | L | (AABD- | L | Ig (Ig | TCR- | TM | IC |
| | | | L)n | | linker) | ConP | | |
| 2 | SP | L | (AABD- | L | IgCL | CD16- | CD16- | CD16- |
| | | | L)n | | | Hinge | TM | CP |
| | SP | L | (AABD- | L | Ig- | CD16- | CD16- | CD16- |
| | | | L)n | | CH1 | Hinge | TM | CP |
| 3 | SP | L | (AABD- | L | IgCL | CD16- | CD16- | CD16- |
| | | | L)n | | | Hinge | TM | CP |
| | SP | L | (AABD- | L | IgG1- | CD16- | CD16- | CD16- |
| | | | L)n | | CH1 | Hinge | TM | CP |
| 4 | SP | L | (AABD- | L | IgCL | CD16- | CD16- | CD16- |
| | | | L)n | | | Hinge | TM | CP |
| | SP | L | (AABD- | L | IgA1- | CD16- | CD16- | CD16- |
| | | | L)n | | CH1 | Hinge | TM | CP |
| 5 | SP | L | (AABD- | L | IgCL | CD16- | CD16- | CD16- |
| | | | L)n | | | Hinge | TM | CP |
| | SP | L | (AABD- | L | IgD- | CD16- | CD16- | CD16- |
| | | | L)n | | CH1 | Hinge | TM | CP |
| 6 | SP | L | (AABD- | L | IgCL | CD16- | CD16- | CD16- |
| | | | L)n | | | Hinge | TM | CP |
| | SP | L | (AABD- | L | IgM- | CD16- | CD16- | CD16- |
| | | | L)n | | CH1 | Hinge | TM | CP |
| 7 | SP | L | (AABD- | L | Ig (Ig | CD16- | CD16- | CD16- |
| | | | L)n | | linker) | ECD | TM | CP |
| | SP | L | (AABD- | L | Ig (Ig | CD16- | CD16- | CD16- |
| | | | L)n | | linker) | ECD | TM | CP |
| 8 | SP | L | (AABD- | L | IgCL | CD16- | CD16- | CD16- |
| | | | L)n | | | ECD | TM | CP |
| | SP | L | (AABD- | L | Ig- | CD16- | CD16- | CD16- |
| | | | L)n | | CH1 | ECD | TM | CP |
| 9 | SP | L | (AABD- | L | IgCL | CD16- | CD16- | CD16- |
| | | | L)n | | | ECD | TM | CP |
| | SP | L | (AABD- | L | IgA1- | CD16- | CD16- | CD16- |
| | | | L)n | | CH1 | ECD | TM | CP |
| 10 | SP | L | (AABD- | L | IgCL | CD16- | CD16- | CD16- |
| | | | L)n | | | ECD | TM | CP |
| | SP | L | (AABD- | L | IgD- | CD16- | CD16- | CD16- |
| | | | L)n | | CH1 | ECD | TM | CP |
| 11 | SP | L | (AABD- | L | IgCL | CD16- | CD16- | CD16- |
| | | | L)n | | | ECD | TM | CP |
| | SP | L | (AABD- | L | IgM- | CD16- | CD16- | CD16- |
| | | | L)n | | CH1 | ECD | TM | CP |

TABLE A1-7

| SAR | | | | | | | | | |
|---|---|---|---|---|---|---|---|---|---|
| Class | | | | | | | | | |
| 1 | SP | L | (AABD- | vL | L | CD16- | CD16- | CD16- | |
| | | | L)n | | | Hinge | TM | CP | |
| | SP | L | (AABD- | vH | L | CD16- | CD16- | CD16- | |
| | | | L)n | | | Hinge | TM | CP | |
| 2 | SP | L | (AABD- | vL | L | CD16- | CD16- | CD16- | |
| | | | L)n | | | ECD | TM | CP | |
| | SP | L | (AABD- | vH | L | CD16- | CD16- | CD16- | |
| | | | L)n | | | ECD | TM | CP | |
| 3 | SP | L | (AABD- | vL | L | Nkp30- | Nkp30- | Nkp30- | |
| | | | L)n | | | Hinge | TM | CP | |
| | SP | L | (AABD- | vH | L | Nkp30- | Nkp30- | Nkp30- | |
| | | | L)n | | | Hinge | TM | CP | |
| 4 | SP | L | (AABD- | vL | L | Nkp30- | Nkp30- | Nkp30- | Nkp30- |
| | | | L)n | | | Ig | Hinge | TM | CP |
| | SP | L | (AABD- | vH | L | Nkp30- | Nkp30- | Nkp30- | Nkp30- |
| | | | L)n | | | Ig | Hinge | TM | CP |
| 5 | SP | L | (AABD- | vL | L | Nkp44- | Nkp44- | Nkp44- | |
| | | | L)n | | | Hinge | TM | CP | |
| | SP | L | (AABD- | vH | L | Nkp44- | Nkp44- | Nkp44- | |
| | | | L)n | | | Hinge | TM | CP | |
| 6 | SP | L | (AABD- | vL | L | Nkp44- | Nkp44- | Nkp44- | Nkp44- |
| | | | L)n | | | Ig | Hinge | TM | CP |
| | SP | L | (AABD- | vH | L | Nkp44- | Nkp44- | Nkp44- | Nkp44- |
| | | | L)n | | | Ig | Hinge | TM | CP |
| 7 | SP | L | (AABD- | vL | L | Nkp46- | Nkp46- | Nkp46- | |
| | | | L)n | | | Hinge | TM | CP | |
| | SP | L | (AABD- | vH | L | Nkp46- | Nkp46- | Nkp46- | |
| | | | L)n | | | Hinge | TM | CP | |
| 8 | SP | L | (AABD- | vL | L | Nkp46- | Nkp46- | Nkp46- | Nkp46- |
| | | | L)n | | | Ig1-Ig2 | Hinge | TM | CP |
| | SP | L | (AABD- | vH | L | Nkp46- | Nkp46- | Nkp46- | Nkp46- |
| | | | L)n | | | Ig1-Ig2 | Hinge | TM | CP |
| 9 | SP | L | (AABD- | vL | L | Dap10- | Dap10- | Dap10- | |
| | | | L)n | | | Hinge | TM | CP | |
| | SP | L | (AABD- | vH | L | Dap10- | Dap10- | Dap10- | |
| | | | L)n | | | Hinge | TM | CP | |

TABLE A1-8

| SAR | | | | | | | | | |
|---|---|---|---|---|---|---|---|---|---|
| Class | | | | | | | | | |
| 1 | SP | L | (AABD- | vL | Ig (Ig | Hinge | TM | CP | |
| | | | L)n | | linker) | | | | |
| | SP | L | (AABD- | vH | Ig (Ig | Hinge | TM | CP | |
| | | | L)n | | linker) | | | | |
| 2 | SP | L | (AABD- | vL | IgCL | CD16- | CD16- | CD16- | |
| | | | L)n | | | Hinge | TM | CP | |
| | SP | L | (AABD- | vH | Ig- | CD16- | CD16- | CD16- | |
| | | | L)n | | CH1 | Hinge | TM | CP | |
| 3 | SP | L | (AABD- | vL | IgCL | CD16- | CD16- | CD16- | |
| | | | L)n | | | ECD | TM | CP | |
| | SP | L | (AABD- | vH | Ig- | CD16- | CD16- | CD16- | |
| | | | L)n | | CH1 | ECD | TM | CP | |
| 4 | SP | L | (AABD- | vL | IgCL | Nkp30- | Nkp30- | Nkp30- | |
| | | | L)n | | | Hinge | TM | CP | |
| | SP | L | (AABD- | vH | Ig- | Nkp30- | Nkp30- | Nkp30- | |
| | | | L)n | | CH1 | Hinge | TM | CP | |
| 5 | SP | L | (AABD- | vL | IgCL | Nkp30- | Nkp30- | Nkp30- | Nkp30- |
| | | | L)n | | | Ig | Hinge | TM | CP |
| | SP | L | (AABD- | vH | Ig- | Nkp30- | Nkp30- | Nkp30- | Nkp30- |
| | | | L)n | | CH1 | Ig | Hinge | TM | CP |
| 6 | SP | L | (AABD- | vL | IgCL | Nkp44- | Nkp44- | Nkp44- | |
| | | | L)n | | | Hinge | TM | CP | |
| | SP | L | (AABD- | vH | Ig- | Nkp44- | Nkp44- | Nkp44- | |
| | | | L)n | | CH1 | Hinge | TM | CP | |
| 7 | SP | L | (AABD- | vL | IgCL | Nkp44- | Nkp44- | Nkp44- | Nkp44- |
| | | | L)n | | | Ig | Hinge | TM | CP |
| | SP | L | (AABD- | vH | Ig- | Nkp44- | Nkp44- | Nkp44- | Nkp44- |
| | | | L)n | | CH1 | Ig | Hinge | TM | CP |
| 8 | SP | L | (AABD- | vL | IgCL | Nkp46- | Nkp46- | Nkp46- | |
| | | | L)n | | | Hinge | TM | CP | |
| | SP | L | (AABD- | vH | Ig- | Nkp46- | Nkp46- | Nkp46- | |
| | | | L)n | | CH1 | Hinge | TM | CP | |
| 9 | SP | L | (AABD- | vL | IgCL | Nkp46- | Nkp46- | Nkp46- | Nkp46- |
| | | | L)n | | | Ig1-Ig2 | Hinge | TM | CP |
| | SP | L | (AABD- | vH | Ig- | Nkp46- | Nkp46- | Nkp46- | Nkp46- |
| | | | L)n | | CH1 | Ig1-Ig2 | Hinge | TM | CP |
| 10 | SP | L | (AABD- | vL | IgCL | Dap10- | Dap10- | Dap10- | |
| | | | L)n | | | Hinge | TM | CP | |
| | SP | L | (AABD- | vH | Ig- | Dap10- | Dap10- | Dap10- | |
| | | | L)n | | CH1 | Hinge | TM | CP | |
| 11 | SP | L | (AABD- | vL | IgCL | Dap10- | Dap10- | Dap10- | CD3ZCP |
| | | | L)n | | | Hinge | TM | CP | |
| | SP | L | (AABD- | vH | Ig- | Dap10- | Dap10- | Dap10- | CD3ZCP |
| | | | L)n | | CH1 | Hinge | TM | CP | |

TABLE A1-9

| SAR | | | | | | | | | |
|---|---|---|---|---|---|---|---|---|---|
| Class | | | | | | | | | |
| 1 | SP | L | (AABD- | vL | L | CD16- | CD16- | CD16- | |
| | | | L)n | | | Hinge | TM | CP | |
| | SP | L | (AABD- | vH | L | Nkp30- | Nkp30- | Nkp30- | |
| | | | L)n | | | Hinge | TM | CP | |
| 2 | SP | L | (AABD- | vL | L | CD16- | CD16- | CD16- | |
| | | | L)n | | | ECD | TM | CP | |
| | SP | L | (AABD- | vH | L | Nkp30- | Nkp30- | Nkp30- | |
| | | | L)n | | | ECD | TM | CP | |
| 3 | SP | L | (AABD- | vL | L | Nkp30- | Nkp30- | Nkp30- | |
| | | | L)n | | | Hinge | TM | CP | |
| | SP | L | (AABD- | vH | L | Nkp44- | Nkp44- | Nkp44- | |
| | | | L)n | | | Hinge | TM | CP | |
| 4 | SP | L | (AABD- | vL | L | Nkp30- | Nkp30- | Nkp30- | Nkp30- |
| | | | L)n | | | Ig | Hinge | TM | CP |
| | SP | L | (AABD- | vH | L | Nkp44- | Nkp44- | Nkp44- | Nkp44- |
| | | | L)n | | | Ig | Hinge | TM | CP |
| 5 | SP | L | (AABD- | vL | L | Nkp44- | Nkp44- | Nkp44- | |
| | | | L)n | | | Hinge | TM | CP | |
| | SP | L | (AABD- | vH | L | Nkp46- | Nkp46- | Nkp46- | |
| | | | L)n | | | Hinge | TM | CP | |
| 6 | SP | L | (AABD- | vL | L | Nkp44- | Nkp44- | Nkp44- | Nkp44- |
| | | | L)n | | | Ig | Hinge | TM | CP |
| | SP | L | (AABD- | vH | L | Nkp46- | Nkp46- | Nkp46- | Nkp46- |
| | | | L)n | | | Ig1-Ig2 | Hinge | TM | CP |
| 7 | SP | L | (AABD- | vL | L | CD16- | CD16- | CD16- | |
| | | | L)n | | | Hinge | TM | CP | |
| | SP | L | (AABD- | vH | L | Nkp46- | Nkp46- | Nkp46- | |
| | | | L)n | | | Hinge | TM | CP | |
| 8 | SP | L | (AABD- | vL | L | CD16- | CD16- | CD16- | |
| | | | L)n | | | Hinge | TM | CP | |
| | SP | L | (AABD- | vH | L | CD3z- | CD3z- | CD3z- | |
| | | | L)n | | | Hinge | TM | CP | |
| 9 | SP | L | (AABD- | vL | L | CD3z- | CD3z- | CD3z- | |
| | | | L)n | | | Hinge | TM | CP | |
| | SP | L | (AABD- | vH | L | Dap10- | Dap10- | Dap10- | |
| | | | L)n | | | Hinge | TM | CP | |
| 10 | SP | L | (AABD- | vL | L | CD16- | CD16- | CD16- | |
| | | | L)n | | | ECD | TM | CP | |
| | SP | L | (AABD- | vH | L | C-abgd (TCRα/β/γ/δ | | | |
| | | | L)n | | | constant chain) | | | |
| 11 | SP | L | (AABD- | vH | L | CD16- | CD16- | CD16- | |
| | | | L)n | | | ECD | TM | CP | |
| | SP | L | (AABD- | vL | L | C-abgd (TCRα/β/γ/δ | | | |
| | | | L)n | | | constant chain) | | | |
| 12 | SP | L | (AABD- | vL | L | Nkp30- | Nkp30- | Nkp30- | |
| | | | L)n | | | Ig | TM | CP | |
| | SP | L | (AABD- | vH | L | C-abgd (TCRα/β/γ/δ | | | |
| | | | L)n | | | constant chain) | | | |
| 13 | SP | L | (AABD- | vH | L | NKp30- | Nkp30- | Nkp30- | |
| | | | L)n | | | Ig | TM | CP | |
| | SP | L | (AABD- | vL | L | C-abgd (TCRα/β/γ/δ | | | |
| | | | L)n | | | constant chain) | | | |
| 14 | SP | L | (AABD- | vL | L | Nkp44- | Nkp44- | Nkp44- | |
| | | | L)n | | | Ig | TM | CP | |
| | SP | L | (AABD- | vH | L | C-abgd (TCRα/β/γ/δ | | | |
| | | | L)n | | | constant chain) | | | |
| 15 | SP | L | (AABD- | vH | L | NKp44- | Nkp44- | Nkp44- | |
| | | | L)n | | | Ig | TM | CP | |
| | SP | L | (AABD- | vL | L | C-abgd (TCRα/β/γ/δ | | | |
| | | | L)n | | | constant chain) | | | |
| 14 | SP | L | (AABD- | vL | L | Dap10- | Dap10- | Dap10- | |
| | | | L)n | | | Hinge | TM | CP | |
| | SP | L | (AABD- | vH | L | C-abgd (TCRα/β/γ/δ | | | |
| | | | L)n | | | constant chain) | | | |
| 15 | SP | L | (AABD- | vH | L | Dap10- | Dap10- | Dap10- | |
| | | | L)n | | | Hinge | TM | CP | |
| | SP | L | (AABD- | vL | L | C-abgd (TCRα/β/γ/δ | | | |
| | | | L)n | | | constant chain) | | | |
| 16 | SP | L | (AABD- | vH | L | CD28- | NKG2D- | 2B4- | CD3z- |
| | | | L)n | | | Hinge | TM | CSD | CP |
| | SP | L | (AABD- | vL | L | CD16- | CD16- | CD16- | |
| | | | L)n | | | Hinge | TM | CP | |
| 17 | SP | L | (AABD- | vH | L | CD28- | NKG2D- | 2B4- | CD3z- |
| | | | L)n | | | Hinge | TM | CSD | CP |
| | SP | L | (AABD- | vL | L | CD3z- | CD3z- | CD3z- | |
| | | | L)n | | | Hinge | TM | CP | |
| 18 | SP | L | (AABD- | vH | L | CD8- | NKG2D- | 2B4- | CD3z- |
| | | | L)n | | | Hinge | TM | CSD | CP |
| | SP | L | (AABD- | vL | L | CD8- | NKG2D- | 2B4- | CD3z- |
| | | | L)n | | | Hinge | TM | CSD | CP |
| 19 | SP | L | (AABD- | vH | L | CD8- | NKG2D- | 2B4- | CD3z- |
| | | | L)n | | | Hinge | TM | CSD | CP |
| | SP | L | (AABD- | vL | L | CD16- | CD16- | CD16- | |
| | | | L)n | | | Hinge | TM | CP | |
| 20 | SP | L | (AABD- | vL | IgCL | Dap10- | Dap10- | Dap10- | CD3z- |
| | | | L)n | | | Hinge | TM | CP | CP |
| | SP | L | (AABD- | vH | Ig- | CD16- | CD16- | CD16- | |
| | | | L)n | | CH1 | Hn | TM | CP | |

TABLE A1-10

| SAR | | | | | | | | | |
|---|---|---|---|---|---|---|---|---|---|
| Class | | | | | | | | | |
| 1 | SP | L | (AABD- | vL | Ig | Hinge | TM | CP | |
| | | | L)n | | linker | | | | |
| | SP | L | (AABD- | vH | Ig | Hinge2 | TM2 | CP2 | |
| | | | L)n | | linker-2 | | | | |
| 1 | SP | L | (AABD- | vL | Ig | CD16- | CD16- | CD16- | |
| | | | L)n | | linker | Hinge | TM | CP | |
| | SP | L | (AABD- | vH | Ig | Nkp30- | Nkp30- | Nkp30- | |
| | | | L)n | | linker | Hinge | TM | CP | |
| 2 | SP | L | (AABD- | vL | IgCL | CD16- | CD16- | CD16- | |
| | | | L)n | | | ECD | TM | CP | |
| | SP | L | (AABD- | vH | IgG1- | Nkp30- | Nkp30- | Nkp30- | |
| | | | L)n | | CH1 | ECD | TM | CP | |
| 3 | SP | L | (AABD- | vL | IgCL | Nkp30- | Nkp30- | Nkp30- | |
| | | | L)n | | | Hinge | TM | CP | |
| | SP | L | (AABD- | vH | IgG1- | Nkp44- | Nkp44- | Nkp44- | |
| | | | L)n | | CH1 | Hinge | TM | CP | |
| 4 | SP | L | (AABD- | vL | IgCL | Nkp30- | Nkp30- | Nkp30- | Nkp30- |
| | | | L)n | | | Ig | Hinge | TM | CP |
| | SP | L | (AABD- | vH | IgG1- | Nkp44- | Nkp44- | Nkp44- | Nkp44- |
| | | | L)n | | CH1 | Ig | Hinge | TM | CP |
| 5 | SP | L | (AABD- | vL | IgCL | Nkp44- | Nkp44- | Nkp44- | |
| | | | L)n | | | Hinge | TM | CP | |
| | SP | L | (AABD- | vH | IgG1- | Nkp46- | Nkp46- | Nkp46- | |
| | | | L)n | | CH1 | Hinge | TM | CP | |
| 6 | SP | L | (AABD- | vL | IgCL | Nkp44- | Nkp44- | Nkp44- | Nkp44- |
| | | | L)n | | | Ig | Hinge | TM | CP |
| | SP | L | (AABD- | vH | IgG1- | Nkp46- | Nkp46- | Nkp46- | Nkp46- |
| | | | L)n | | CH1 | Ig1-Ig2 | Hinge | TM | CP |
| 7 | SP | L | (AABD- | vL | IgCL | CD16- | CD16- | CD16- | |
| | | | L)n | | | Hinge | TM | CP | |
| | SP | L | (AABD- | vH | IgG1- | Nkp46- | Nkp46- | Nkp46- | |
| | | | L)n | | CH1 | Hinge | TM | CP | |
| 8 | SP | L | (AABD- | vL | IgCL | CD16- | CD16- | CD16- | |
| | | | L)n | | | Hinge | TM | CP | |
| | SP | L | (AABD- | vH | IgG1- | CD3z- | CD3z- | CD3z- | |
| | | | L)n | | CH1 | Hinge | TM | CP | |
| 9 | SP | L | (AABD- | vL | IgCL | CD3z- | CD3z- | CD3z- | |
| | | | L)n | | | Hinge | TM | CP | |
| | SP | L | (AABD- | vH | IgG1- | Dap10- | Dap10- | Dap10- | |
| | | | L)n | | CH1 | Hinge | TM | CP | |
| 10 | SP | L | (AABD- | vL | IgCL | CD16- | CD16- | CD16- | |
| | | | L)n | | | ECD | TM | CP | |
| | SP | L | (AABD- | vH | IgG1- | C-abgd (TCRα/β/γ/δ | | | |
| | | | L)n | | CH1 | constant chain) | | | |
| 11 | SP | L | (AABD- | vH | IgCL | CD16- | CD16- | CD16- | |
| | | | L)n | | | ECD | TM | CP | |
| | SP | L | (AABD- | vL | IgG1- | C-abgd (TCRα/β/γ/δ | | | |
| | | | L)n | | CH1 | constant chain) | | | |
| 12 | SP | L | (AABD- | vL | IgCL | Nkp30- | Nkp30- | Nkp30- | |
| | | | L)n | | | Ig | TM | CP | |
| | SP | L | (AABD- | vH | IgG1- | C-abgd (TCRα/β/γ/δ | | | |
| | | | L)n | | CH1 | constant chain) | | | |
| 13 | SP | L | (AABD- | vH | IgCL | NKp30- | Nkp30- | Nkp30- | |
| | | | L)n | | | Ig | TM | CP | |
| | SP | L | (AABD- | vL | IgG1- | C-abgd (TCRα/β/γ/δ | | | |
| | | | L)n | | CH1 | constant chain) | | | |
| 14 | SP | L | (AABD- | vL | IgCL | Nkp44- | Nkp44- | Nkp44- | |
| | | | L)n | | | Ig | TM | CP | |
| | SP | L | (AABD- | vH | IgG1- | C-abgd (TCRα/β/γ/δ | | | |
| | | | L)n | | CH1 | constant chain) | | | |
| 15 | SP | L | (AABD- | vH | IgG1- | NKp44- | Nkp44- | Nkp44- | |
| | | | L)n | | CH1 | Ig | TM | CP | |
| | SP | L | (AABD- | vL | IgCL | C-abgd (TCRα/β/γ/δ | | | |
| | | | L)n | | | constant chain) | | | |
| 14 | SP | L | (AABD- | vL | IgG1- | Dap10- | Dap10- | Dap10- | |
| | | | L)n | | CH1 | Hinge | TM | CP | |
| | SP | L | (AABD- | vH | IgCL | C-abgd (TCRα/β/γ/δ | | | |
| | | | L)n | | | constant chain) | | | |
| 15 | SP | L | (AABD- | vH | IgG1- | Dap10- | Dap10- | Dap10- | |
| | | | L)n | | CH1 | Hinge | TM | CP | |

| | | | | | |
|---|---|---|---|---|---|
| SP | L | (AABD- | vL | IgCL | C-abgd (TCRα/β/γ/δ |
| | | L)n | | | constant chain) |

TABLE A1-11
EXEMPLARY TCR-SAR WITH BACKBONE OF zSAR

| SAR | | | | | | | | | | |
|---|---|---|---|---|---|---|---|---|---|---|
| Class | | | | | | | | | | |
| 1 | SP | L | (AABD- | Va | L | Ig | ECD | TM | CP | |
| | | | L)n | | | | | | | |
| | SP | L | (AABD- | Vb | L | Ig | ECD | TM | CP | |
| | | | L)n | | | | | | | |
| 2 | SP | L | (AABD- | Va | L | Ig | Hinge | TM | CP | |
| | | | L)n | | | | | | | |
| | SP | L | (AABD- | Vb | L | Ig | Hinge | TM | CP | |
| | | | L)n | | | | | | | |
| 3 | SP | L | (AABD- | Va | L | Ig | CD3z- | CD3z- | CD3z-CP | |
| | | | L)n | | | | ECD | TM | | |
| | SP | L | (AABD- | Vb | L | Ig (Ig | CD16- | CD16- | CD16-CP | |
| | | | L)n | | | linker) | ECD | TM | | |
| 4 | SP | L | (AABD- | Va | L | TCR- | CD3z- | CD3z- | CD3z-CP | |
| | | | L)n | | | Ig | ECD | TM | | |
| | SP | L | (AABD- | Vb | L | TCR- | CD16- | CD16- | CD16-CP | |
| | | | L)n | | | Ig | hinge | TM | | |
| 5 | SP | L | (AABD- | Va | L | TCRa- | CD3z- | CD3z- | CD3z-CP | |
| | | | L)n | | | Ig3 | ECD | TM | | |
| | SP | L | (AABD- | Vb | L | TCRb- | CD16- | CD16- | CD16-CP | |
| | | | L)n | | | Ig3 | hinge | TM | | |
| 6 | SP | L | (AABD- | Va | L | TCRa- | CD3z- | CD3z- | CD3z-CP | |
| | | | L)n | | | Ig3 | ECD | TM | | |
| | SP | L | (AABD- | Vb | L | TCRb- | CD3z- | CD16- | CD16-CP | |
| | | | L)n | | | Ig3 | ECD | TM | | |
| 7 | SP | L | (AABD- | Va | L | TCRa- | CD3z- | CD3z- | CD3z-CP | |
| | | | L)n | | | Ig3 | ECD | TM | | |
| | SP | L | (AABD- | Vb | L | TCRb- | NKp30- | NKp30- | NKp30-CP | |
| | | | L)n | | | Ig3 | hinge | TM | | |
| 8 | SP | L | (AABD- | Va | L | TCRa- | CD3z- | CD3z- | CD3z-CP | |
| | | | L)n | | | Ig3 | ECD | TM | | |
| | SP | L | (AABD- | Vb | L | TCRb- | CD3z- | NKp30- | NKp30-CP | |
| | | | L)n | | | Ig3 | ECD | TM | | |
| 9 | SP | L | (AABD- | Va | L | TCRa- | CD3z- | CD3z- | CD3z-CP | |
| | | | L)n | | | Ig3 | ECD | TM | | |
| | SP | L | (AABD- | Vb | L | TCRb- | NKp46- | NKp46- | NKp46-CP | |
| | | | L)n | | | Ig3 | hinge | TM | | |
| 10 | SP | L | (AABD- | Va | L | TCRa- | CD3z- | CD3z- | CD3z-CP | |
| | | | L)n | | | Ig3 | ECD | TM | | |
| | SP | L | (AABD- | Vb | L | TCRb- | DAP10- | NKp44- | NKp44-CP | |
| | | | L)n | | | Ig3 | ECD | TM | | |
| 11 | SP | L | (AABD- | Va | L | Ig | ECD | TM | CSD | CP |
| | | | L)n | | | | | | | |
| | SP | L | (AABD- | Vb | L | Ig | ECD | TM | CSD | CP |
| | | | L)n | | | | | | | |
| 12 | SP | L | (AABD- | Va | L | TCR- | CD3z- | CD3z- | 4-1BB | CD3z- |
| | | | L)n | | | Ig | ECD | TM | | CP |
| | SP | L | (AABD- | Vb | L | TCR- | CD3z- | CD3z- | 4-1BB | CD3z- |
| | | | L)n | | | Ig | ECD | TM | | CP |
| 13 | SP | L | (AABD- | Va | L | TCR- | CD3z- | CD3z- | CD3z- | |
| | | | L)n | | | Ig | ECD | TM | CP | |
| | SP | L | (AABD- | Vb | L | TCR- | CD3z- | CD3z- | 4-1BB | CD3z- |
| | | | L)n | | | Ig | ECD | TM | | CP |

TABLE A1-12
EXEMPLARY TCR-SAR WITH BACKBONE OF zSAR

| SAR | | | | | | | | | | |
|---|---|---|---|---|---|---|---|---|---|---|
| Class | | | | | | | | | | |
| 1 | SP | L | (AABD- | Va | L | Ig | ECD | TM | CP | |
| | | | L)n | | | | | | | |
| | SP | L | (AABD- | Vb | L | Ig | ECD | TM | CP | |
| | | | L)n | | | | | | | |
| 2 | SP | L | (AABD- | Va | L | Ig | Hinge | TM | CP | |
| | | | L)n | | | | | | | |
| | SP | L | (AABD- | Vb | L | Ig | Hinge | TM | CP | |
| | | | L)n | | | | | | | |
| 3 | SP | L | (AABD- | Va | L | Ig | CD3z- | CD3z- | CD3z-CP | |
| | | | L)n | | | | ECD | TM | | |
| | SP | L | (AABD- | Vb | L | Ig (Ig | CD16- | CD16- | CD16-CP | |
| | | | L)n | | | linker) | ECD | TM | | |
| 4 | SP | L | (AABD- | Va | L | TCR- | CD3z- | CD3z- | CD3z-CP | |
| | | | L)n | | | Ig | ECD | TM | | |
| | SP | L | (AABD- | Vb | L | TCR- | CD16- | CD16- | CD16-CP | |
| | | | L)n | | | Ig | hinge | TM | | |
| 5 | SP | L | (AABD- | Va | L | TCRa- | CD3z- | CD3z- | CD3z-CP | |
| | | | L)n | | | Ig3 | ECD | TM | | |
| | SP | L | (AABD- | Vb | L | TCRb- | CD16- | CD16- | CD16-CP | |
| | | | L)n | | | Ig3 | hinge | TM | | |
| 6 | SP | L | (AABD- | Va | L | TCRa- | CD3z- | CD3z- | CD3z-CP | |
| | | | L)n | | | Ig3 | ECD | TM | | |
| | SP | L | (AABD- | Vb | L | TCRb- | CD3z- | CD16- | CD16-CP | |
| | | | L)n | | | Ig3 | ECD | TM | | |
| 7 | SP | L | (AABD- | Va | L | TCRa- | CD3z- | CD3z- | CD3z-CP | |
| | | | L)n | | | Ig3 | ECD | TM | | |
| | SP | L | (AABD- | Vb | L | TCRb- | NKp30- | NKp30- | NKp30-CP | |
| | | | L)n | | | Ig3 | hinge | TM | | |
| 8 | SP | L | (AABD- | Va | L | TCRa- | CD3z- | CD3z- | CD3z-CP | |
| | | | L)n | | | Ig3 | ECD | TM | | |
| | SP | L | (AABD- | Vb | L | TCRb- | CD3z- | NKp30- | NKp30-CP | |
| | | | L)n | | | Ig3 | ECD | TM | | |
| 9 | SP | L | (AABD- | Va | L | TCRa- | CD3z- | CD3z- | CD3z-CP | |
| | | | L)n | | | Ig3 | ECD | TM | | |
| | SP | L | (AABD- | Vb | L | TCRb- | NKp46- | NKp46- | NKp46-CP | |
| | | | L)n | | | Ig3 | Hn | TM | | |
| 10 | SP | L | (AABD- | Va | L | TCRa- | CD3z- | CD3z- | CD3z-CP | |
| | | | L)n | | | Ig3 | ECD | TM | | |
| | SP | L | (AABD- | Vb | L | TCRb- | DAP10- | NKp44- | NKp44-CP | |
| | | | L)n | | | Ig3 | ECD | TM | | |
| 11 | SP | L | (AABD- | Va | L | Ig | ECD | TM | CSD | CP |
| | | | L)n | | | | | | | |
| | SP | L | (AABD- | Vb | L | Ig | ECD | TM | CSD | CP |
| | | | L)n | | | | | | | |
| 12 | SP | L | (AABD- | Va | L | TCR- | CD3z- | CD3z- | 4-1BB | CD3z- |
| | | | L)n | | | Ig | ECD | TM | | CP |
| | SP | L | (AABD- | Vb | L | TCR- | CD3z- | CD3z- | 4-1BB | CD3z- |
| | | | L)n | | | Ig | ECD | TM | | CP |
| 13 | SP | L | (AABD- | Va | L | TCR- | CD3z- | CD3z- | CD3z- | |
| | | | L)n | | | Ig | ECD | TM | CP | |
| | SP | L | (AABD- | Vb | L | TCR- | CD3z- | CD3z- | 4-1BB | CD3z- |
| | | | L)n | | | Ig | ECD | TM | | CP |

TABLE A1-13
EXEMPLARY SAR

| SP | L | (AABD- | (scFv- | L | CD16- | CD16- | CD8- | CD8- | 4-1BB | CD3z |
|---|---|---|---|---|---|---|---|---|---|---|
| | | L)n | L)n | | D1 | D2 | Hn | TM | | |
| SP | L | (AABD- | (scFv- | L | CD16- | CD16- | CD28- | CD28- | CD28 | CD3z |
| | | L)n | L)n | | D1 | D2 | Hn | TM | | |
| SP | L | | (scFv- | L | CD16- | CD16- | CD28- | CD28- | CD28 | CD3z |
| | | | L)n | | D1 | D2 | Hn | TM | | |
| SP | L | (AABD- | | L | CD16- | CD16- | CD28- | CD28- | CD28 | CD3z |
| | | L)n | | | D1 | D2 | Hn | TM | | |
| SP | L | (AABD- | (scFv- | L | CD16- | CD16- | CD16- | CD28- | CD28 | CD3z |
| | | L)n | L)n | | D1 | D2 | Hn | TM | | |
| SP | L | | (scFv- | L | CD16- | CD16- | CD16- | CD28- | CD28 | CD3z |
| | | | L)n | | D1 | D2 | Hn | TM | | |
| SP | L | | scTCR | L | CD16- | CD16- | CD16- | CD28- | CD28 | CD3z |
| | | | | | D1 | D2 | Hn | TM | | |
| SP | L | (AABD- | (scFv- | L | CD16- | CD16- | 2B4- | 2B4- | 2B4 | CD3z |
| | | L)n | L)n | | D1 | D2 | Hn | TM | | |
| SP | L | | (scFv- | L | CD16- | CD16- | 2B4- | 2B4- | 2B4 | CD3z |
| | | | L)n | | D1 | D2 | Hn | TM | | |
| SP | L | (AABD- | | L | CD16- | CD16- | 2B4- | 2B4- | 2B4 | CD3z |
| | | L)n | | | D1 | D2 | Hn | TM | | |
| SP | L | | scTCR | L | CD16- | CD16- | CD16- | 2B4- | 2B4 | CD3z |
| | | | | | D1 | D2 | Hn | TM | | |
| SP | L | (AABD- | (scFv- | L | CD64- | CD64- | CD16- | CD16- | CD16-CP | |
| | | L)n | L)n | | D1 | D2-3 | Hn | TM | | |
| SP | L | (AABD- | | L | CD64- | CD64- | CD64- | CD16- | CD16-CP | |
| | | L)n | | | D1 | D2-3 | Hn | TM | | |
| SP | L | | (scFv- | L | CD64- | CD64- | CD64- | CD16- | CD16-CP | |
| | | | L)n | | D1 | D2-3 | Hn | TM | | |
| SP | L | | scTCR | L | CD64- | CD64- | CD16- | CD16- | CD16-CP | |
| | | | | | D1 | D2-3 | Hn | TM | | |
| SP | L | (AABD- | (scFv- | L | CD64- | CD64- | CD64- | CD16- | CD16-CP | |
| | | L)n | L)n | | D1 | D2-3 | Hn | TM | | |
| SP | L | (AABD- | (scFv- | L | CD32- | CD32- | CD16- | CD16- | CD16-CP | |
| | | L)n | L)n | | D1 | D2 | Hn | TM | | |
| SP | L | (AABD- | (scFv- | L | CD32- | CD32- | CD32- | CD16- | CD16-CP | |
| | | L)n | L)n | | D1 | D2 | Hn | TM | | |
| SP | L | (AABD- | (scFv- | L | CD32- | CD32- | CD16- | CD16- | CD16-CP | |
| | | L)n | L)n | | D1 | D2 | Hn | TM | | |
| SP | L | (AABD- | (scFv- | L | Nkp30- | Nkp30- | Nkp30- | CD3z | | |
| | | L)n | L)n | | Ig | Hi | TM | | | |
| SP | L | (AABD- | (scFv- | L | Nkp30- | Nkp30- | Nkp30- | 2B4 | CD3z | |
| | | L)n | L)n | | Ig | Hn | TM | | | |
| SP | L | (AABD- | (scFv- | L | Nkp30- | Nkp30- | Nkp30- | 2B4 | Nkp30 | |
| | | L)n | L)n | | Ig | Hn | TM | | | |
| SP | L | (AABD- | (scFv- | L | Nkp30- | Nkp30- | 2B4- | 2B4 | Nkp30 | |
| | | L)n | L)n | | Ig | Hn | TM | | | |
| SP | L | (AABD- | (scFv- | L | Nkp30- | 2B4- | 2B4- | 2B4 | CD3z | |
| | | L)n | L)n | | Ig | Hn | TM | | | |
| SP | L | (AABD- | (scFv- | L | Nkp46- | Nkp46- | CD28- | CD28- | CD28 | CD3z |
| | | L)n | L)n | | Ig1 | Ig2 | Hn | TM | | |

TABLE A1-14
EXEMPLARY TCR-SAR WITH BACKBONE OF zSAR

| SAR | | | | | | | | | | |
|---|---|---|---|---|---|---|---|---|---|---|
| Class | | | | | | | | | | |
| 1 | SP | L | (AABD- | Va | L | IgCL | CD3z- | CD3z- | CD3z-CP | |
| | | | L)n | | | | ECD | TM | | |
| | SP | L | (AABD- | Vb | L | Ig- | CD3z- | CD3z- | CD3z-CP | |
| | | | L)n | | | CH1 | ECD | TM | | |
| 2 | SP | L | (AABD- | Va | L | IgCL | CD3z- | CD3z- | CD3z-CP | |
| | | | L)n | | | | ECD | TM | | |
| | SP | L | (AABD- | Vb | L | IgG1- | CD3z- | CD3z- | CD3z-CP | |
| | | | L)n | | | CH1 | ECD | TM | | |
| 3 | SP | L | (AABD- | Va | L | IgCL | CD3z- | CD3z- | 4-1BB | CD3z-CP |
| | | | L)n | | | | ECD | TM | | |
| | SP | L | (AABD- | Vb | L | IgG1- | CD3z- | CD3z- | 4-1BB | CD3z-CP |
| | | | L)n | | | CH1 | ECD | TM | | |
| 4 | SP | L | (AABD- | Va | L | TCR- | CD3z- | CD3z- | CD3z-CP | |
| | | | L)n | | | Ig | ECD | TM | | |
| | SP | L | (AABD- | Vb | L | TCR- | CD3z- | CD3z- | CD3z-CP | |
| | | | L)n | | | Ig | ECD | TM | | |
| 5 | SP | L | (AABD- | Va | L | TCRa- | CD3z- | CD3z- | CD3z-CP | |
| | | | L)n | | | Ig3 | ECD | TM | | |
| | SP | L | (AABD- | Vb | L | TCRb- | CD3z- | CD3z- | CD3z-CP | |
| | | | L)n | | | Ig3 | ECD | TM | | |
| 6 | SP | L | (AABD- | Va | L | TCRa- | CD3z- | CD3z- | 4-1BB | CD3z-CP |
| | | | L)n | | | Ig3 | ECD | TM | | |
| | SP | L | (AABD- | Vb | L | TCRb- | CD3z- | CD3z- | 4-1BB | CD3z-CP |
| | | | L)n | | | Ig3 | ECD | TM | | |
| 7 | SP | L | (AABD- | Va | L | TCRa- | CD3z- | CD3z- | CD28 | CD3z-CP |
| | | | L)n | | | Ig3 | ECD | TM | | |
| | SP | L | (AABD- | Vb | L | TCRb- | CD3z- | CD3z- | CD28 | CD3z-CP |
| | | | L)n | | | Ig3 | ECD | TM | | |
| 8 | SP | L | (AABD- | Va | L | TCRa- | CD3z- | CD3z- | 41BB | CD3z-CP |
| | | | L)n | | | Ig3 | ECD | TM | | |
| | SP | L | (AABD- | Vb | L | TCRb- | CD3z- | CD3z- | CD28 | CD3z-CP |
| | | | L)n | | | Ig3 | ECD | TM | | |
| 9 | SP | L | (AABD- | Vg | L | Ig (Ig | CD3z- | CD3z- | CD3z-CP | |
| | | | L)n | | | linker) | ECD | TM | | |
| | SP | L | (AABD- | Vd | L | Ig (Ig | CD3z- | CD3z- | CD3z-CP | |
| | | | L)n | | | linker) | ECD | TM | | |
| 10 | SP | L | (AABD- | Vg | L | IgCL | CD3z- | CD3z- | CD3z-CP | |
| | | | L)n | | | | ECD | TM | | |
| | SP | L | (AABD- | Vd | L | Ig- | CD3z- | CD3z- | 41BB | CD3z-CP |
| | | | L)n | | | CH1 | ECD | TM | | |
| 11 | SP | L | (AABD- | Vg | L | TCRg- | CD3z- | CD3z- | CD28 | CD3z-CP |
| | | | L)n | | | Ig3 | ECD | TM | | |
| | SP | L | (AABD- | Vd | L | TCRd- | CD3z- | CD3z- | CD3z-CP | |
| | | | L)n | | | Ig3 | ECD | TM | | |

TABLE A1-15
EXEMPLARY TCR-SAR WITH BACKBONE OF zSAR

| SAR | | | | | | | | | |
|---|---|---|---|---|---|---|---|---|---|
| Class | | | | | | | | | |
| 1 | SP | L | (AABD- | Va | L | Ig (Ig | ECD | TM | CP |
| | | | L)n | | | linker) | | | |
| | SP | L | (AABD- | Vb | L | Ig (Ig | ECD | TM | CP |
| | | | L)n | | | linker) | | | |
| 2 | SP | L | (AABD- | Va | L | Ig (Ig | Hinge | TM | CP |
| | | | L)n | | | linker) | | | |
| | SP | L | (AABD- | Vb | L | Ig (Ig | Hinge | TM | CP |
| | | | L)n | | | linker) | | | |
| 3 | SP | L | (AABD- | Va | L | Ig (Ig | CD3z- | CD3z- | CD3z-CP |
| | | | L)n | | | linker) | ECD | TM | |
| | SP | L | (AABD- | Vb | L | Ig (Ig | CD3z- | CD3z- | CD3z-CP |
| | | | L)n | | | linker) | ECD | TM | |
| 4 | SP | L | (AABD- | Va | L | IgCL | CD3z- | CD3z- | CD3z-CP |
| | | | L)n | | | | ECD | TM | |
| | SP | L | (AABD- | Vb | L | Ig- | CD3z- | CD3z- | CD3z-CP |
| | | | L)n | | | CH1 | ECD | TM | |
| 5 | SP | L | (AABD- | Va | L | IgCL | CD3z- | CD3z- | CD3z-CP |
| | | | L)n | | | | ECD | TM | |
| | SP | L | (AABD- | Vb | L | IgG1- | CD3z- | CD3z- | CD3z-CP |
| | | | L)n | | | CH1 | ECD | TM | |
| 6 | SP | L | (AABD- | Va | L | IgCL | CD3z- | CD3z- | CD3z-CP |
| | | | L)n | | | | ECD | TM | |
| | SP | L | (AABD- | Vb | L | IgA1- | CD3z- | CD3z- | CD3z-CP |
| | | | L)n | | | CH1 | ECD | TM | |
| 7 | SP | L | (AABD- | Va | L | IgCL | CD3z- | CD3z- | CD3z-CP |
| | | | L)n | | | | ECD | TM | |
| | SP | L | (AABD- | Vb | L | IgD- | CD3z- | CD3z- | CD3z-CP |
| | | | L)n | | | CH1 | ECD | TM | |
| 8 | SP | L | (AABD- | Va | L | IgCL | CD3z- | CD3z- | CD3z-CP |
| | | | L)n | | | | ECD | TM | |
| | SP | L | (AABD- | Vb | L | IgM- | CD3z- | CD3z- | CD3z-CP |
| | | | L)n | | | CH1 | ECD | TM | |
| 9 | SP | L | (AABD- | Vg | L | Ig (Ig | ECD | TM | CP |
| | | | L)n | | | linker) | | | |
| | SP | L | (AABD- | Vd | L | Ig (Ig | ECD | TM | CP |
| | | | L)n | | | linker) | | | |
| 10 | SP | L | (AABD- | Vg | L | Ig (Ig | Hinge | TM | CF |
| | | | L)n | | | linker) | | | |
| | SP | L | (AABD- | Vd | L | Ig (Ig | Hinge | TM | CP |
| | | | L)n | | | linker) | | | |
| 11 | SP | L | (AABD- | Vg | L | Ig (Ig | CD3z- | CD3z- | CD3z-CP |
| | | | L)n | | | linker) | ECD | TM | |
| | SP | L | (AABD- | Vd | L | Ig (Ig | CD3z- | CD3z- | CD3z-CP |
| | | | L)n | | | linker) | ECD | TM | |
| 12 | SP | L | (AABD- | Vg | L | IgCL | CD3z- | CD3z- | CD3z-CP |
| | | | L)n | | | | ECD | TM | |
| | SP | | (AABD- | Vd | L | Ig- | CD3z- | CD3z- | CD3z-CP |
| | | | L)n | | | CH1 | ECD | TM | |
| 13 | SP | L | (AABD- | Vg | L | IgCL | CD3z- | CD3z- | CD3z-CP |
| | | | L)n | | | | ECD | TM | |
| | SP | L | (AABD- | Vd | L | IgA1- | CD3z- | CD3z- | CD3z-CP |
| | | | L)n | | | CH1 | ECD | TM | |
| 14 | SP | L | (AABD- | Vg | L | IgCL | CD3z- | CD3z- | CD3z-CP |
| | | | L)n | | | | ECD | TM | |
| | SP | L | (AABD- | Vd | L | IgD- | CD3z- | CD3z- | CD3z-CP |
| | | | L)n | | | CH1 | ECD | TM | |
| 15 | SP | L | (AABD- | Vg | L | IgCL | CD3z- | CD3z- | CD3z-CP |
| | | | L)n | | | | ECD | TM | |
| | SP | L | (AABD- | Vd | L | IgM- | CD3z- | CD3z- | CD3z-CP |
| | | | L)n | | | CH1 | ECD | TM | |

TABLE A1-16
EXEMPLARY TCR-SAR

| | | | | | | | | | | |
|---|---|---|---|---|---|---|---|---|---|---|
| 1 | SP | L | (AABD- | Va | L | Ig (Ig | ECD | TM | CSD | CP (AD) |
| | | | L)n | | | linker) | | | | |
| | SP | L | (AABD- | Vb | L | Ig (Ig | ECD | TM | CSD | CP (AD) |
| | | | L)n | | | linker) | | | | |
| 2 | SP | L | (AABD- | Va | L | Ig (Ig | Hinge | TM | CSD | CP (AD) |
| | | | L)n | | | linker) | | | | |
| | SP | L | (AABD- | Vb | L | Ig (Ig | Hinge | TM | CSD | CP (AD) |
| | | | L)n | | | linker) | | | | |
| 3 | SP | L | (AABD- | Va | L | Ig (Ig | CD3z- | CD3z- | BB | CD3z-CP |
| | | | L)n | | | linker) | ECD | TM | | |
| | SP | L | (AABD- | Vb | L | Ig (Ig | CD3z- | CD3z- | BB | CD3z-CP |
| | | | L)n | | | linker) | ECD | TM | | |
| 4 | SP | L | (AABD- | Va | L | IgCL | CD3z- | CD3z- | BB | CD3z-CP |
| | | | L)n | | | | ECD | TM | | |
| | SP | L | (AABD- | Vb | L | IgG1- | CD3z- | CD3z- | BB | CD3z-CP |
| | | | L)n | | | CH1 | ECD | TM | | |
| 5 | SP | L | (AABD- | Va | L | IgCL | CD3z- | CD3z- | CD28 | CD3z-CP |
| | | | L)n | | | | ECD | TM | | |
| | SP | L | (AABD- | Vb | L | IgG1- | CD3z- | CD3z- | CD28 | CD3z-CP |
| | | | L)n | | | CH1 | ECD | TM | | |
| 6 | SP | L | (AABD- | Va | L | IgCL | CD3z- | CD3z- | 2B4 | CD3z-CP |
| | | | L)n | | | | ECD | TM | | |
| | SP | L | (AABD- | Vb | L | IgG1- | CD3z- | CD3z- | 2B4 | CD3z-CP |
| | | | L)n | | | CH1 | ECD | TM | | |
| 7 | SP | L | (AABD- | Va | L | IgCL | CD3z- | CD3z- | OX40 | CD3z-CP |
| | | | L)n | | | | ECD | TM | | |
| | SP | L | (AABD- | Vb | L | IgG1- | CD3z- | CD3z- | OX40 | CD3z-CP |
| | | | L)n | | | CH1 | ECD | TM | | |
| 8 | SP | L | (AABD- | Va | L | Ig (Ig | CD3z- | CD3z- | CD3z-CP | |
| | | | L)n | | | linker) | ECD | TM | | |
| | SP | L | (AABD- | Vb | L | Ig (Ig | CD3z- | CD3z- | BB | CD3z-CP |
| | | | L)n | | | linker) | ECD | TM | | |

TABLE A1-17
EXEMPLARY TCR-SAR

| | | | | | | | | | |
|---|---|---|---|---|---|---|---|---|---|
| SP | L | (AABD- | Vg | L | Ig (Ig | CD3z- | CD3z- | CD3z-CP | |
| | | L)n | | | linker) | ECD | TM | | |
| SP | L | (AABD- | Vd | L | Ig (Ig | CD3z- | CD3z- | CD3z-CP | |
| | | L)n | | | linker) | ECD | TM | | |
| SP | L | (AABD- | Vg | L | TCRg- | CD3z- | CD3z- | CD3z-CP | |
| | | L)n | | | Ig3 | ECD | TM | | |
| SP | L | (AABD- | Vd | L | TCRd- | CD3z- | CD3z- | CD3z-CP | |
| | | L)n | | | Ig3 | ECD | TM | | |
| SP | L | (AABD- | Vg | L | IgCL | CD3z- | CD3z- | CD3z-CP | |
| | | L)n | | | | ECD | TM | | |
| SP | L | (AABD- | Vd | L | IgA1- | CD3z- | CD3z- | CD3z-CP | |
| | | L)n | | | CH1 | ECD | TM | | |
| SP | L | (AABD- | Vg | L | IgCL | CD3z- | CD3z- | CD3z-CP | |
| | | L)n | | | | ECD | TM | | |
| SP | L | (AABD- | Vd | L | IgD- | CD3z- | CD3z- | CD3z-CP | |
| | | L)n | | | CH1 | ECD | TM | | |
| SP | L | (AABD- | Vg | L | IgCL | CD3z- | CD3z- | CD3z-CP | |
| | | L)n | | | | ECD | TM | | |
| SP | L | (AABD- | Vd | L | IgM- | CD3z- | CD3z- | CD3z-CP | |
| | | L)n | | | CH1 | ECD | TM | | |
| SP | L | (AABD- | Va | L | Ig (Ig | CD3z- | CD3z- | 4-1BB | z |
| | | L)n | | | linker) | ECD | TM | | |
| SP | L | (AABD- | Vb | L | Ig (Ig | CD3z- | CD3z- | 4-1BB | z |
| | | L)n | | | linker) | ECD | TM | | |
| SP | L | (AABD- | Va | L | IgCL | CD3z- | CD3z- | 4-1BB | z |
| | | L)n | | | | ECD | TM | | |
| SP | L | (AABD- | Vb | L | IgG1- | CD3z- | CD3z- | 4-1BB | z |
| | | L)n | | | CH1 | ECD | TM | | |
| SP | L | (AABD- | Va | L | IgCL | CD3z- | CD3z- | CD28 | z |
| | | L)n | | | | ECD | TM | | |
| SP | L | (AABD- | Vb | L | IgG1- | CD3z- | CD3z- | CD28 | z |
| | | L)n | | | CH1 | ECD | TM | | |
| SP | L | (AABD- | Va | L | IgCL | CD3z- | CD3z- | 2B4 | z |
| | | L)n | | | | ECD | TM | | |
| SP | L | (AABD- | Vb | L | IgG1- | CD3z- | CD3z- | 2B4 | z |
| | | L)n | | | CH1 | ECD | TM | | |
| SP | L | (AABD- | Va | L | IgCL | CD3z- | CD3z- | OX40 | z |
| | | L)n | | | | ECD | TM | | |
| SP | L | (AABD- | Vb | L | IgG1- | CD3z- | CD3z- | OX40 | z |
| | | L)n | | | CH1 | ECD | TM | | |
| SP | L | (AABD- | Va | L | Ig (Ig | CD3z- | CD3z- | z | |
| | | L)n | | | linker) | ECD | TM | | |
| SP | L | (AABD- | Vb | L | Ig (Ig | CD3z- | CD3z- | 4-1BB | z |
| | | L)n | | | linker) | ECD | TM | | |

TABLE A1-18
EXEMPLARY TCR-SAR WITH BACKBONE OF zSAR

| SAR | | | | | | | | | |
|---|---|---|---|---|---|---|---|---|---|
| Class | | | | | | | | | |
| 1 | SP | L | (AABD- | Va | L | Ig (Ig | ECD | TM | CP |
| | | | L)n | | | linker) | | | |
| | SP | L | (AABD- | Vb | L | Ig (Ig | ECD | TM | CP |
| | | | L)n | | | linker) | | | |
| 2 | SP | L | (AABD- | Va | L | Ig (Ig | Hinge | TM | CP |
| | | | L)n | | | linker) | | | |
| | SP | L | (AABD- | Vb | L | Ig (Ig | Hinge | TM | CP |
| | | | L)n | | | linker) | | | |
| 3 | SP | L | (AABD- | Va | L | Ig (Ig | CD3z- | CD3z- | CD3z-CP |
| | | | L)n | | | linker) | ECD | TM | |
| | SP | L | (AABD- | Vb | L | Ig (Ig | CD16- | CD16- | CD16-CP |
| | | | L)n | | | linker) | ECD | TM | |
| 4 | SP | L | (AABD- | Va | L | TCR- | CD3z- | CD3z- | CD3z-CP |
| | | | L)n | | | Ig | ECD | TM | |
| | SP | L | (AABD- | Vb | L | TCR- | CD16- | CD16- | CD16-CP |
| | | | L)n | | | Ig | hinge | TM | |
| 5 | SP | L | (AABD- | Va | L | TCRa- | CD3z- | CD3z- | CD3z-CP |
| | | | L)n | | | Ig3 | ECD | TM | |
| | SP | L | (AABD- | Vb | L | TCRb- | CD16- | CD16- | CD16-CP |
| | | | L)n | | | Ig3 | hinge | TM | |
| 6 | SP | L | (AABD- | Va | L | TCRa- | CD3z- | CD3z- | CD3z-CP |
| | | | L)n | | | Ig3 | ECD | TM | |
| | SP | L | (AABD- | Vb | L | TCRb- | CD3z- | CD16- | CD16-CP |
| | | | L)n | | | Ig3 | ECD | TM | |
| 7 | SP | L | (AABD- | Va | L | TCRa- | CD3z- | CD3z- | CD3z-CP |
| | | | L)n | | | Ig3 | ECD | TM | |
| | SP | L | (AABD- | Vb | L | TCRb- | NKp30- | NKp30- | NKp30-CP |
| | | | L)n | | | Ig3 | hinge | TM | |
| 8 | SP | L | (AABD- | Va | L | TCRa- | CD3z- | CD3z- | CD3z-CP |
| | | | L)n | | | Ig3 | ECD | TM | |
| | SP | L | (AABD- | Vb | L | TCRb- | NKp44- | NKp44- | NKp44-CP |
| | | | L)n | | | Ig3 | hinge | TM | |
| 9 | SP | L | (AABD- | Va | L | TCRa- | CD3z- | CD3z- | CD3z-CP |
| | | | L)r | | | Ig3 | ECD | TM | |
| | SP | L | (AABD- | Vb | L | TCRb- | NKp46- | NKp46- | NKp46-CP |
| | | | L)n | | | Ig3 | hinge | TM | |
| 10 | SP | L | (AABD- | Va | L | TCRa- | CD3z- | CD3z- | CD3z-CP |
| | | | L)n | | | Ig3 | ECD | TM | |
| | SP | L | (AABD- | Vb | L | TCRb- | CD3z- | NKp30- | NKp30-CP |
| | | | L)n | | | Ig3 | ECD | TM | |
| 11 | SP | L | (AABD- | Va | L | TCRa- | CD3z- | CD3z- | CD3z-CP |
| | | | L)n | | | Ig3 | ECD | TM | |
| | SP | L | (AABD- | Vb | L | TCRb- | DAP10- | NKp44- | NKp44-CP |
| | | | L)n | | | Ig3 | ECD | TM | |
| 12 | SP | L | (AABD- | Va | L | TCRa- | CD3z- | CD3z- | CD3z-CP |
| | | | L)n | | | Ig3 | ECD | TM | |
| | SP | L | (AABD- | Vb | L | TCRb- | DAP12- | NKp46- | NKp46-CP |
| | | | L)n | | | Ig3 | ECD | TM | |
| 13 | SP | L | (AABD- | Va | L | TCRa- | FCRG- | FCRG- | FCRG-CP |
| | | | L)n | | | Ig3 | ECD | TM | |
| | SP | L | (AABD- | Vb | L | TCRb- | CD16- | CD16- | CD16-CP |
| | | | L)n | | | Ig3 | hinge | TM | |
| 14 | SP | L | (AABD- | Va | L | TCRa- | FCRG- | FCRG- | FCRG-CP |
| | | | L)n | | | Ig3 | ECD | TM | |
| | SP | L | (AABD- | Vb | L | TCRb- | CD3z- | CD3z- | CD3z-CP |
| | | | L)n | | | Ig3 | ECD | TM | |
| 15 | SP | L | (AABD- | Va | L | TCRa- | CD3z- | CD3z- | CD3z-CP |
| | | | L)n | | | Ig3 | ECD | TM | |
| | SP | L | (AABD- | Vb | L | TCRb- | FCRG- | FCRG- | FCRG-CP |
| | | | L)n | | | Ig3 | ECD | TM | |
| 16 | SP | L | (AABD- | Va | L | TCRa- | DAP10- | DAP10- | DAP10-CP |
| | | | L)n | | | Ig3 | ECD | TM | |
| | SP | L | (AABD- | Vb | L | TCRb- | DAP10- | DAP10- | DAP10-CP |
| | | | L)n | | | Ig3 | ECD | TM | |
| 17 | SP | L | (AABD- | Va | L | TCRa- | CD3z- | CD3z- | CD3z-CP |
| | | | L)n | | | Ig3 | ECD | TM | |
| | SP | L | (AABD- | Vb | L | TCRb- | DAP10- | DAP10- | DAP10-CP |
| | | | L)n | | | Ig3 | ECD | TM | |
| 18 | SP | L | (AABD- | Va | L | TCRa- | DAP10- | DAP10- | DAP10-CP |
| | | | L)n | | | Ig3 | ECD | TM | |
| | SP | L | (AABD- | Vb | L | TCRb- | CD3z- | CD3z- | CD3z-CP |
| | | | L)n | | | Ig3 | ECD | TM | |
| 19 | SP | L | (AABD- | Va | L | TCRa- | DAP10- | DAP10- | DAP10-CP |
| | | | L)n | | | Ig3 | ECD | TM | |
| | SP | L | (AABD- | V | L | TCRb- | FCRG- | FCRG- | FCRG-CP |
| | | | L)n | b | | Ig3 | ECD | TM | |
| 20 | SP | L | (AABD- | Va | L | TCRa- | FCRG- | FCRG- | FCRG-CP |
| | | | L)n | | | Ig3 | ECD | TM | |
| | SP | L | (AABD- | Vb | L | TCRb- | DAP10- | DAP10- | DAP10-CP |
| | | | L)r | | | Ig3 | ECD | TM | |
| 21 | SP | L | (AABD- | Va | L | TCRa- | DAP12- | DAP12- | DAP12-CP |
| | | | L)n | | | Ig3 | ECD | TM | |
| | SP | L | (AABD- | Vb | L | TCRb- | DAP12- | DAP12- | DAP12-CP |
| | | | L)n | | | Ig3 | ECD | TM | |
| 22 | SP | L | (AABD- | Va | L | TCRa- | CD3z- | CD3z- | CD3z-CP |
| | | | L)n | | | Ig3 | ECD | TM | |
| | SP | L | (AABD- | Vb | L | TCRb- | DAP12- | DAP12- | DAP12-CP |
| | | | L)n | | | Ig3 | ECD | TM | |
| 23 | SP | L | (AABD- | Va | L | TCRa- | DAP12- | DAP12- | DAP12-CP |
| | | | L)n | | | Ig3 | ECD | TM | |
| | SP | L | (AABD- | Vb | L | TCRb- | CD3z- | CD3z- | CD3z-CP |
| | | | L)n | | | Ig3 | ECD | TM | |
| 24 | SP | L | (AABD- | Va | L | TCRa- | DAP12- | DAP12- | DAP12-CP |
| | | | L)n | | | Ig3 | ECD | TM | |
| | SP | L | (AABD- | Vb | L | TCRb- | FCRG- | FCRG- | FCRG-CP |
| | | | L)n | | | Ig3 | ECD | TM | |
| 25 | SP | L | (AABD- | Va | L | TCRa- | FCRG- | FCRG- | FCRG-CP |
| | | | L)n | | | Ig3 | ECD | TM | |
| | SP | L | (AABD- | Vb | L | TCRb- | DAP12- | DAP12- | DAP12-CP |
| | | | L)n | | | Ig3 | ECD | TM | |
| 26 | SP | L | (AABD- | Va | L | TCRa- | DAP10- | DAP10- | DAP10-CP |
| | | | L)n | | | Ig3 | ECD | TM | |
| | SP | L | (AABD- | Vb | L | TCRb- | DAP12- | DAP12- | DAP12-CP |
| | | | L)n | | | Ig3 | ECD | TM | |

TABLE A1-19
EXEMPLARY TCR-SAR

| | | | | | | | | | | |
|---|---|---|---|---|---|---|---|---|---|---|
| SP | L | (AABD- | (scFv-L)n | L | CD16- | CD16- | CD8- | CD8- | BB | Z |
| | | L)n | | | D1 | D2 | Hn | TM | | |
| SP | L | (AABD- | (scFv-L)n | L | CD16- | CD16- | CD28- | CD28- | 28 | Z |
| | | L)n | | | D1 | D2 | Hn | TM | | |
| SP | L | | (scFv-L)n | L | CD16- | CD16- | CD28- | CD28- | 28 | Z |
| | | | | | D1 | D2 | Hn | TM | | |
| SP | L | (AABD- | | L | CD16- | CD16- | CD28- | CD28- | 28 | Z |
| | | L)n | | | D1 | D2 | Hn | TM | | |
| SP | L | | scTCR | L | CD16- | CD16- | CD16- | CD28- | 28 | Z |
| | | | | | D1 | D2 | Hn | TM | | |
| SP | L | (AABD- | (scFv-L)n | L | CD16- | CD16- | 2B4- | 2B4- | 2B4 | Z |
| | | L)n | | | D1 | D2 | Hn | TM | | |
| SP | L | | (scFv-L)n | L | CD16- | CD16- | 2B4- | 2B4- | 2B4 | Z |
| | | | | | D1 | D2 | Hn | TM | | |
| SP | L | (AABD- | | L | CD16- | CD16- | 2B4- | 2B4- | 2B4 | Z |
| | | L)n | | | D1 | D2 | Hn | TM | | |
| SP | L | | scTCR | L | CD16- | CD16- | CD16- | 2B4- | 2B4 | Z |
| | | | | | D1 | D2 | Hn | TM | | |
| SP | L | (AABD- | (scFv-L)n | L | Nkp30- | Nkp30- | Nkp30- | z | | |
| | | L)n | | | Ig | Hi | TM | | | |
| SP | L | | (scFv-L)n | L | Nkp30- | Nkp30- | Nkp30- | 2B4 | z | |
| | | | | | Ig | Hn | TM | | | |
| SP | L | (AABD- | (scFv-L)n | L | Nkp30- | Nkp30- | Nkp30- | 2B4 | Nkp30 | |
| | | L)n | | | Ig | Hn | TM | | | |
| SP | L | (AABD- | (scFv-L)n | L | Nkp30- | Nkp30- | 2B4- | 2B4 | Nkp30 | |
| | | L)n | | | Ig | Hn | TM | | | |
| SP | L | (AABD- | (scFv-L)n | L | Nkp30- | 2B4- | 2B4- | 2B4 | z | |
| | | L)n | | | Ig | Hn | TM | | | |
| SP | L | (AABD- | (scFv-L)n | L | Nkp30- | CD28- | CD28- | CD28 | z | |
| | | L)n | | | Ig | Hn | TM | | | |
| SP | L | (AABD- | (scFv-L)n | L | Nkp44- | Nkp44- | Nkp44- | | z | |
| | | L)n | | | Ig | Hn | TM | | | |
| SP | L | (AABD- | (scFv-L)n | L | Nkp44- | Nkp44- | Nkp44- | 2B4 | z | |
| | | L)n | | | Ig | Hn | TM | | | |
| SP | L | (AABD- | (scFv-L)n | L | Nkp44- | Nkp44- | Nkp44- | 2B4 | Nkp30 | |
| | | L)n | | | Ig | Hn | TM | | | |
| SP | L | (AABD- | (scFv-L)n | L | Nkp44- | Nkp44- | 2B4- | 2B4 | Nkp30 | |
| | | L)n | | | Ig | Hn | TM | | | |
| SP | L | (AABD- | (scFv-L)n | L | Nkp44- | 2B4- | 2B4- | 2B4 | z | |
| | | L)n | | | Ig | Hn | TM | | | |
| SP | L | (AABD- | (scFv-L)n | L | Nkp44- | CD28- | CD28- | CD28 | z | |
| | | L)n | | | Ig | Hn | TM | | | |
| SP | L | (AABD- | (scFv-L)n | L | Nkp46- | Nkp46- | CD28- | CD28- | 28 | z |
| | | L)n | | | Ig1 | Ig2 | Hn | TM | | |
| SP | L | | (scFv-L)n | L | Nkp46- | Nkp46- | CD28- | CD28- | 28 | z |
| | | | | | Ig1 | Ig2 | Hn | TM | | |
| SP | L | (AABD- | | L | Nkp46- | Nkp46- | CD28- | CD28- | 28 | z |
| | | L)n | | | Ig1 | Ig2 | Hn | TM | | |
| SP | L | | scTCR | L | Nkp46- | Nkp46- | NKp46- | CD28- | 28 | z |
| | | | | | Ig1 | Ig2 | Hn | TM | | |
| SP | L | (AABD- | (scFv-L)n | L | Nkp46- | Nkp46- | 2B4- | 2B4- | 2B4 | z |
| | | L)n | | | Ig1 | Ig2 | Hn | TM | | |
| SP | L | | (scFv-L)n | L | Nkp46- | Nkp46- | 2B4- | 2B4- | 2B4 | z |
| | | | | | Ig1 | Ig2 | Hn | TM | | |
| SP | L | (AABD- | | L | Nkp46- | Nkp46- | 2B4- | 2B4- | 2B4 | z |
| | | L)n | | | Ig1 | Ig2 | Hn | TM | | |
| SP | L | | scTCR | L | Nkp46- | Nkp46- | CD16- | 2B4- | 2B4 | z |
| | | | | | Ig1 | Ig2 | Hn | TM | | |

As used herein “beneficial results” or “desired results” may include, but are not limited to, lessening or alleviating the severity of the disease condition, preventing the disease condition from worsening, curing the disease condition, preventing the disease condition from developing, lowering the chances of a patient developing the disease condition and prolonging a patient's life or life expectancy.

“Binds the same epitope as” means the ability of an antibody, scFv, or other antigen binding domain to bind to a target antigen and having the same epitope as an exemplified antibody, scFv, or other antigen binding domain. As an example, the epitopes of the exemplified antibody, scFv, or other binding agent and other antibodies can be determined using standard epitope mapping techniques. Epitope mapping techniques, well known in the art include Epitope Mapping Protocols in Methods in Molecular Biology, Vol. 66 (Glenn E. Morris, Ed., 1996) Humana Press, Totowa, New Jersey. Exemplary epitopes of human CD20, BCMA and human MPL antigen bound by scFv, SARs, antibodies and other immunotherapeutics of the current disclosure are provided in SEQ ID NO: 15149-15154, 15155-15159 and 15160, respectively of patent application PCT/US18/53247, which is incorporated in its entirety by reference herein.

It is to be inferred without explicit recitation and unless otherwise intended, that when the disclosure relates to a polypeptide, protein, polynucleotide, antibody, SAR or fragment thereof, an equivalent or a biologically equivalent of such is intended within the scope of this disclosure. As used herein, the term “biological equivalent thereof” or “variant” or “functional variant” is intended to be synonymous with “equivalent thereof” when referring to a reference protein, antibody or fragment thereof, receptor of fragment thereof, ligand or fragment thereof, non-immunoglobulin antigen binding domain or fragment thereof, SAR or a fragment thereof, polypeptide or nucleic acid, intends those having minimal homology while still maintaining desired structure or functionality. Unless specifically recited herein, it is contemplated that any of the above also includes equivalents thereof, including alternatively spliced isoforms and equivalents from other animal species. For example, an equivalent intends at least about 70% homology or identity, or at least 80% homology or identity and alternatively, or at least about 85%, or alternatively at least about 90%, or alternatively at least about 95%, or alternatively at least 98% percent homology or identity and exhibits substantially equivalent biological activity to the reference protein, polypeptide, antibody or fragment thereof or nucleic acid. Alternatively, when referring to polynucleotides, an equivalent thereof is a polynucleotide that hybridizes under stringent conditions to the reference polynucleotide or its complement. Alternatively, when referring to polypeptides or proteins, an equivalent thereof is an expressed polypeptide or protein from a polynucleotide that hybridizes under stringent conditions to the polynucleotide or its complement that encodes the reference polypeptide or protein.

It will be recognized that proteins can have identity or homology to one another and retain similar or identical functions. A polypeptide “variant,” as used herein, is a polypeptide that differs from the recited polypeptide only in conservative substitutions and/or modifications, such that therapeutic, antigenic and/or immunogenic properties of the polypeptide are retained. Polypeptide variants typically exhibit at least about 70%, more typically at least about 90% and most typically at least about 95% homology to the identified polypeptides. For polypeptides with immunoreactive properties, variants can, alternatively, be identified by modifying the amino acid sequence of one of the above polypeptides, and evaluating the immunoreactivity of the modified polypeptide. Such modified sequences can be prepared and tested using, for example, the representative procedures described herein. The disclosure includes SAR and SAR components (e.g., extracellular, hinge, transmembrane and cytosolic regions of CD16, CD32, CD64, FcRγ, DAP10, DAP12, DNAM1, OX40, 2B4, KIR2DL1, KIR2DS4, NKp30, NKp44, NKp46, NKG2D, NKG2A, NKG2C, NKG2E, NKG2F, NKG2H, TCRα, TCRβ, TCRγ, TCRδ, and CD3z etc.) that have at least 70%, 80%, 85%, 90%, 95%, 97%, 98%, 98.5%, 99% or 99.9% identity to any of the amino acid sequences described herein while retaining the biological activity. The disclosure also includes antigen binding domains, extracellular domains, hinge domains, transmembrane domains, cytosolic domains, costimulatory domains, accessory modules that have at least 70%, 80%, 85%, 90%, 95%, 97%, 98%, 98.5%, 99% or 99.9% identity to any of the sequences described herein while retaining the biological activity. Variants include homologs from other species and alternative spliced isoforms.

As used herein, the term “CD3 complex” refers to a cell surface molecule assembly comprising numerous proteins for transmembrane signaling of TCR activation.

As used herein, the term “CDR” or “complementarity determining region” is intended to mean the non-contiguous antigen combining sites found within the variable region of both heavy and light chain polypeptides. These particular regions have been described by Kabat et al., J. Bio. Chem. 252:6609-6616 (1977); Kabat et al., U.S. Dept. of Health and Human Services, “Sequences of proteins of immunological interest” (1991); Chothia et al., J. Mol. Bio. 196:901-917 (1987); and MacCallum et al., J. Mol. Bio. 25 262:732-745 (1996), where the definitions include overlapping or subsets of amino acid residues when compared against each other. Nevertheless, application of either definition to refer to a CDR of an antibody or grafted antibodies or variants thereof is intended to be within the scope of the term as defined and used herein. As used herein, the different CDRs of an antibody could be also defined by a combination of the different definitions. For example, vHCDR1 could be defined based on Kabat and VHCDR2 could be defined based on Chothia. The amino acid residues which encompass the CDRs as defined by each of the above cited references are as follows:

| | Kabat | Chothia | MacCallum |
|---|---|---|---|
| VHCDR1 | 31-35 | 26-32 | 30-35 |
| VHCDR2 | 50-65 | 53-55 | 47-58 |
| VHCDR3 | 95-102 | 96-10 | 193-101 |
| VLCDR1 | 24-34 | 26-32 | 30-36 |
| VLCDR2 | 50-56 | 50-52 | 46-55 |
| VLCDR3 | 89-97 | 91-96 | 89-96 |

(Residue Numbers correspond to the identified reference).

The SEQ IDs of the CDRs of the exemplary vL and vH segments that can make up antigen binding domains of SAR, bispecific antibodies and other immunotherapeutics of the current disclosure are provided in SEQ ID NO: 13204-14121 and SEQ ID NO: 14122-15039, respectively (Tables 6A, B) of PCT/US2018/053247, in Tables 5-6 of PCT/US2017/064379 and in Table 39 of PCT/US2021/022641, which are incorporated herein by reference. The SEQ IDs of the exemplary vL and vH segments that can make up antigen binding domains of SAR, antibodies and other immunotherapeutics are also provided in Table 3 of the current disclosure. The light chain CDR1, CDR2 and CDR3 of the vL fragments and scFvs provided in the current disclosure (e.g., Table 3) are provided in SEQ ID NO: 10882-11118, 11119-11355 and 11356-11592. The CDR1, CDR2 and CDR3 of the vL fragments provided in the current disclosure (e.g., Table 3) are provided in SEQ ID NO: 10882-11118, 11119-11355 and 11356-11592. The heavy chain CDR1, CDR2 and CDR3 of the vH fragments and scFvs provided in the current disclosure (e.g., Table 3) are provided in SEQ ID NO:11593-11829, 11830-12066, 12067-12303, respectively.

In some embodiments, reference to an antigen-binding module (such as a Fab-like or Fv-like antigen-binding module) that specifically binds to a target antigen means that the antigen-binding module binds to the target antigen with (a) an affinity that is at least about 10 (e.g., about 10, 20, 30, 40, 50, 75,100,200, 300, 400, 500, 750, 1000 or more) times its binding affinity for other molecules; or (b) a Ka no more than about 1/10 (e.g., 1/10, 1/20, 1/30, 1/40, 1/50, 1175, 1/100, 1/200, 1/300, 1/400, 1/500, 1/750, 1/1000 or less) times its Ka for binding to other molecules. Binding affinity can be determined by methods known in the art, such as ELISA, fluorescence activated cell sorting (FACS) analysis, Malibu-Glo assay, Topanga Assay, or radioimmunoprecipitation assay (RIA).

“Cancer” and “cancerous” refer to or describe the physiological condition in mammals that is typically characterized by unregulated cell growth. The terms “tumor” and “cancer” are used interchangeably herein, e.g., both terms encompass solid and liquid, e.g., diffuse or circulating, tumors. As used herein, the term “cancer” or “tumor” includes premalignant, as well as malignant cancers and tumors. The term “cancer” is meant to include all types of cancerous growths or oncogenic processes, metastatic tissues or malignantly transformed cells, tissues, or organs, irrespective of histopathologic type or stage of invasiveness.

“Cell therapy” or “Cell-based therapy” or “Immune cell therapy” or Immune effector cell therapy” refers to a therapy that involves the use of cells for the prevention or treatment of a disease. Non-limiting examples of cell therapy include CAR-T cell therapy, NK-cell therapy, recombinant TCR-T cell therapy, TIL (tumor infiltrating lymphocytes).

“Chemotherapeutic agents” are compounds that are known to be of use in chemotherapy for cancer.

“Chimeric antigen receptors” (CARs) are artificial (non-naturally occurring) immune cell (e.g., T cell) receptors contemplated for use as a therapy for cancer, using a technique called adoptive cell transfer. CARs are also known as artificial T-cell receptors, chimeric T-cell receptors or chimeric immunoreceptors. CARs are constructed specifically to stimulate T cell activation and proliferation in response to a specific antigen to which the CAR binds. Generally, a CAR refers to a set of polypeptides, typically two in the simplest embodiments, which when expressed in an immune effector cell, provides the cell with specificity for a target cell, typically a cancer cell, and with intracellular signal generation. In some embodiments, a CAR comprises at least an extracellular antigen binding domain, a transmembrane domain and a cytoplasmic signaling domain (also referred to herein as “an intracellular signaling domain”) comprising a functional signaling domain derived from a stimulatory molecule and/or costimulatory molecule. In some aspects, the set of polypeptides are contiguous with each other. In one aspect, the stimulatory molecule is the zeta chain associated with the T cell receptor complex. In one aspect, the cytoplasmic signaling domain further comprises one or more functional signaling domains derived from at least one costimulatory molecule as defined below. In one embodiment, the costimulatory molecule is chosen from the costimulatory molecules described herein, e.g., 4-1BB (i.e., CD137), CD27, OX40, 2B4, and/or CD28. In one embodiment, the CAR comprises an optional leader sequence at the amino-terminus (N-ter) of the CAR fusion protein. In one embodiment, the CAR further comprises a leader sequence at the N-terminus of the extracellular antigen binding domain, wherein the leader sequence is optionally cleaved from the antigen binding domain (e.g., a scFv) during cellular processing and localization of the CAR to the cellular membrane. In various embodiments, CARs are recombinant polypeptides comprising an antigen-specific domain (ASD), a hinge region (HR), a transmembrane domain (TMD), an optional co-stimulatory domain (CSD) and an intracellular signaling domain (ISD). The optional costimulatory domain is generally absent in the 1t generation CAR constructs. The nucleic acid and protein sequences of several exemplary 2nd generation CARs comprising the different antigen binding domains (e.g., vL and vH fragments, vHH, ligands and receptors etc.) and incorporating the 41BB costimulatory domain are presented in SEQ ID NO: 1455-1703 and 341-7589 (Table 8) of PCT/US2020/014237.

“Codon optimization” or “controlling for species codon bias” refers to the preferred codon usage of a particular host cell. As will be understood by those of skill in the art, it can be advantageous to modify a coding sequence to enhance its expression in a particular host. Those of skill in the art will recognize that, due to the degenerate nature of the genetic code, a variety of DNA compounds differing in their nucleotide sequences can be used to encode a given polypeptide of the disclosure.

As used herein, “co-express” refers to expression of two or more polynucleotides or genes. Genes may be nucleic acids encoding, for example, a single protein or a chimeric protein as a single polypeptide chain. A SAR (e.g., a CAR, SIR, zSIR, or TCR etc.) described herein may be encoded by a single polynucleotide chain and expressed as single polypeptide chain, which is subsequently cleaved into different polypeptides, each representing a distinct functional unit. In some embodiments, where the SAR consists of two or more functional polypeptide units, the different functional units are coexpressed using one or more polynucleotide chains. In one embodiment, costimulation is provided by an accessory module that is co-expressed with the SAR or a TCR but is not an integral part of the SAR (e.g., a CAR, SIR, zSIR, or TCR etc.) polypeptide. In another embodiment, the different polynucleotide chains are linked by nucleic acid sequences that encode for cleavable linkers (e.g., T2A, F2A, P2A, E2A etc.) (Table 20). In another embodiment, a Ser-Gly-Ser-Gly (SGSG) motif (SEQ ID NO: 1239 and 1240) is also added upstream of the cleavable linker sequences to enhance the efficiency of cleavage. The nucleic acid and amino acid sequences of exemplary cleavable linkers and Furine cleavage sites are provided in Table 20. The polynucleotides encoding the different units of a SAR may be linked by IRES (Internal Ribosomal Entry Site) sequences. In an embodiment, the different functional units (e.g., two or more chains) of a SAR are expressed using a single vector. The different functional units of a SAR may be expressed using a single promoter or multiple promoters. In an embodiment, the different functional units of a SAR are expressed using two or more vectors. The nucleic acid and amino acid sequences of exemplary cleavable linkers and Furine cleavage sites are provided in Table 20.

A “conservative substitution” or “conservative sequence modifications” refers to amino acid modifications that do not significantly affect or alter the binding characteristics or function of the encoded protein. For example, “conservative sequence modifications” refers to amino acid modifications that do not significantly affect or alter the binding characteristics or function of a SAR of the disclosure (e.g., a conservative change in the constant chain, antibody, antibody fragment, or non-immunoglobulin binding domains). Such conservative modifications include amino acid substitutions, additions and deletions. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within a SAR of the disclosure can be replaced with other amino acid residues from the same side chain family and the altered SAR can be tested using the binding and/or functional assays described herein.

A “costimulatory intracellular signaling domain” or “Co-stimulatory domain” or “CSD” as used herein refers to the portion of a SAR which enhances the proliferation, survival and/or development of T cells. The SARs of the disclosure may comprise zero, one or more co-stimulatory domains. Each co-stimulatory domain comprises the costimulatory domain of any one or more of, for example, members of the TNFR superfamily, CD28, CD137 (4-1BB), CD134 (OX40), BAFF-R, HVEM, CD27, CD2, CD5, TNFR-I, TNFR-II, Fas, CD30, CD40 or combinations thereof. Additional exemplary co-stimulatory domains include the signaling domains of 2B4, NKp30, NKp44, NKp46, GITR, CD81, CD160, DAP10 and B7-H3. Other co-stimulatory domains (e.g., from other proteins) will be apparent to those of skill in the art and may be used in connection with alternate embodiments of the disclosure. The co-stimulatory domain can comprise the entire intracellular portion, or the entire native intracellular signaling domain, of the molecule from which it is derived, or a functional fragment or derivative thereof. The SARs of the disclosure may comprise zero, one or more co-stimulatory domains.

The term a “costimulatory molecule” or a “costimulatory receptor” refers to a cognate binding partner on an immune cell (e.g., T cell, NK cell, macrophage, granulocyte, dendritic cell etc.) that specifically binds with a costimulatory ligand, thereby mediating a costimulatory response by the immune cell such as, but not limited to, proliferation, activation or cytokine secretion. Costimulatory extracellular molecules are cell surface molecules other than antigen receptors or their ligands that contribute to an efficient immune response. Costimulatory molecules include, but are not limited to, an MHC class I molecule, BTLA and a Toll ligand receptor, as well as OX40, Dap10, CD27, CD28, CD2, CD5, CD8, ICAM-1, LFA-1 (CD11a/CD18), ICOS (CD278), Lck, TNFR-I, TNFR-II, Fas, CD30, CD40, CD81 and 4-1BB (CD137). A co-stimulatory receptor may be expressed on cells other than T cells, such as NK cells or macrophages.

The term “cTCR” refers to a wild-type TCR nucleic acid coding sequence and the corresponding wild-type TCR protein linked to an antigen binding domain that is not derived from a TCR. cTCR have been described in (Gross, Waks, & Eshhar, 1989). cTCRs are used in some embodiments and as reference controls. For example, a cTCR having a CD19 binding domain and a CD19-SIR (comprising a mutant TCR chain and CD19 binding domain) will have different expression and/or difference binding affinities to the target antigen.

The term “cytosolic” or “cytoplasmic” refers to an agent, e.g., a protein that is situated in the cytoplasm of a cell in its mature form. A cytosolic protein can translocate into the nucleus but is not a transmembrane protein and is not secreted outside the cell.

Cytokine Release Syndrome (CRS) is a complication of cell therapies (e.g., SAR-T, bispecific T cell engaging antibodies etc.) that manifests itself with a constellation of signs and symptoms such as fever, hypotension, shortness of breath, renal dysfunction, pulmonary dysfunction and/or capillary leak syndrome.

The term “degenerative disorders” refers to a disease that is the result of a continuous process based on degenerative cell changes, affecting tissues or organs, which will increasingly deteriorate over time, whether due to normal bodily wear or lifestyle choices such as exercise or eating habits. Exemplary degenerative diseases include Alzheimer's disease, Creutzfeldt-Jakob disease, Diabetes mellitus (type II), and Atherosclerosis.

“Derived from” as that term is used herein, indicates a relationship between a first and a second molecule. It generally refers to structural similarity between the first molecule and a second molecule and does not connotate or include a process or source limitation on a first molecule that is derived from a second molecule. For example, in the case of an antigen binding domain that is derived from an antibody molecule, the antigen binding domain retains sufficient antibody structure such that is has the required function, namely, the ability to bind to an antigen. It does not connotate or include a limitation to a particular process of producing the antibody, e.g., it does not mean that, to provide the antigen binding domain, one must start with an antibody sequence and delete unwanted sequence, or impose mutations, to arrive at the antigen binding domain.

“Dimerization molecule,” as that term is used herein refers to a molecule that promotes the association of a first switch domain with a second switch domain. In embodiments, the dimerization molecule does not naturally occur in the subject, or does not occur in concentrations that would result in significant dimerization. In embodiments, the dimerization molecule is a small molecule, e.g., rapamycin or a rapalogue, e.g., RAD001, Rimiducid or AP20187. Rimiducid can be at about 0.01-1 mg/kg and has an EC50 in cell culture of about 0.1 nM. AP20187 can be administered from about 2-10 mg/kg/day in single or multi-doses.

The phrase “disease associated with expression of a target antigen” or “disease associated antigen as described herein” includes, but is not limited to, a disease associated with expression of a target antigen as described herein or condition associated with cells which express a target antigen as described herein including, e.g., proliferative diseases such as a cancer or malignancy or a precancerous condition such as a myelodysplasia, a myelodysplastic syndrome or myeloproliferative disorder or a pre leukemia; or a noncancer related indication associated with cells which express a target antigen as described herein.

“Disease targeted by genetically modified cells” as used herein encompasses the targeting of any cell involved in any manner in any disease by the genetically modified cells of the disclosure, irrespective of whether the genetically modified cells target diseased cells or healthy cells to effectuate a therapeutically beneficial result.

The term “Dissociation constant (Kd)” is defined as the equilibrium constant of the dissociation of a receptor-ligand (e.g., binding domain—cognate) interaction. In some embodiments, a SAR of the disclosure binds to the target antigen with an equilibrium dissociation constant (Kd) from about 0.1 pM to about 500 nM.

As used herein a “diverse set of non-naturally occurring immune receptors” or “diverse set of SARs” refers to a plurality of non-naturally occurring immune receptors or SARS targeting an antigen. In embodiment, diverse set of SARs have the same binding domain linked to a diverse set of signaling chains or “backbones”. In an embodiment, the diverse set of SARs may possess diverse range of binding affinities to a target antigen. In an embodiment, the diverse set of SARs may exhibit varied expression levels.

As used herein, an “epitope” is defined to be the portion of an antigen capable of eliciting an immune response, or the portion of an antigen that binds to an antibody or antibody fragment. Epitopes can be a protein sequence or subsequence.

As used herein, the term “engager” refers to a molecule, e.g., a fusion polypeptide, which is capable of forming a link between an immune cell (e.g., a T cell, a NK cell, a NKT cell, a B cell, a macrophage, a neutrophil) and a tumor cell that results in activation of the immune cell. Examples of engagers include, but are not limited to, bi-specific T cell engagers (BiTEs), bi specific killer cell engagers (BiKEs), tri-specific killer cell engagers (TRiKE), or multi-specific killer cell engagers, or universal engagers compatible with multiple immune cell types.

The term “expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, including cosmids, plasmids (e.g., naked or contained in liposomes) and viruses (e.g., lentiviruses, retroviruses, adenoviruses, and adeno-associated viruses) that incorporate the recombinant polynucleotide.

A “functional portion” (“biologically active portion”) of a protein (e.g., SAR, IL-2, IL-15 etc.) refers to a portion of a protein that retains one or more functions of full length or mature protein. Such functions for IL-12 or IL-15 include the promotion of NK cell survival, regulation of NK cell and T cell activation and proliferation as well as the support of NK cell development from hematopoietic stem cells.

As used herein, “F(ab)” refers to a fragment of an antibody structure that binds to an antigen but is monovalent and does not have a Fc portion, for example, an antibody digested by the enzyme papain yields two F(ab) fragments and an Fc fragment (e.g., a heavy (H) chain constant region; Fc region that does not bind to an antigen).

As used herein, “F(ab′)2” refers to an antibody fragment generated by pepsin digestion of whole IgG antibodies, wherein this fragment has two antigen binding (ab′) (bivalent) regions, wherein each (ab′) region comprises two separate amino acid chains, a part of a H chain and a light (L) chain linked by an S—S bond for binding an antigen and where the remaining H chain portions are linked together. A “F(ab′)2” fragment can be split into two individual Fab′ fragments.

The term “FcRγ” or “FCER1G” or “FCRG” or “FcRy” as used herein refers to gene represented by Gene ID: 2207. It is a disulfide linker transmembrane signaling adaptor that is part of high affinity IgE receptor and other Fc receptors.

The term “functional portion” when used in reference to a SAR refers to any part or fragment of the SAR, which part or fragment retains the biological activity of the SAR of which it is a part (the parent SAR). Functional portions encompass, for example, those parts of a SAR that retain the ability to recognize target cells, or detect, treat, or prevent a disease, to a similar extent, the same extent, or to a higher extent, as the parent SAR. In reference to the parent SAR, the functional portion can comprise, for instance, about 10%, 25%, 30%, 50%, 68%, 80%, 90%, 95%, or more, of the parent SAR.

The term “flexible polypeptide linker” as used herein refers to a peptide linker that consists of amino acids such as glycine and/or serine residues used alone or in combination, to link polypeptide chains together (e.g., variable heavy and variable light chain regions together). In one embodiment, the flexible polypeptide linker is a Gly/Ser linker and comprises the amino acid sequence (Gly-Gly-Gly-Gly-Ser)ₙ, (e.g., SEQ ID NO:2431) where n is a positive integer equal to or greater than 1. For example, n=l, n=2, n=3. n=4, n=5 and n=6, n=7, n=8, n=9 and n=10. In one embodiment, the flexible polypeptide linkers include, but are not limited to, (Gly₄Ser)₄ or (Gly₄Ser)₃.

“Genetically modified cells”, “redirected cells”, “genetically engineered cells” or “modified cells” as used herein refer to cells that express a SAR of the disclosure. In some embodiments, the genetically modified cells comprise vectors that encode a SAR. In some embodiments, the genetically modified cells comprise vectors that encode a SAR and one or more accessory molecules (e.g., PDL1, PDL2, crmA, MC159 etc.) in the same vector. In some embodiments, the genetically modified cells comprise a first vector that encodes a SAR and a second vector that encodes the accessory molecule. In some embodiments, the genetically modified cells comprise a first vector that encodes a SAR and a second vector that encodes more than one accessory molecule. In some embodiments, the genetically modified cells comprise a first vector that encodes a SAR and a second vector that encodes the first accessory molecule and a third vector that encodes a second accessory molecule.

An “HLA-independent TCR” or an “MHC-independent TCR” as defined herein is a TCR that can recognize an antigen independent of MHC restriction. In an exemplary embodiment, an HLA-independent TCR may bind to an antigen on the cell surface that is not presented by the MHC complex. In an embodiment, an HLA-independent TCR may bind to an antigen that is expressed on the cell surface independent of presentation by the MHC complex. An HLA-independent TCR may be a naturally occurring TCR. In an exemplary embodiment, an HLA-independent TCR is MC.7.G5 (MC7G5) that recognizes MR1, a ubiquitously expressed, monomorphic antigen presenting molecule. An HLA-independent TCR may be an engineered or recombinant TCR. In an exemplary embodiment, an HLA-independent TCR is an engineered TCR that may bind to proteins that are expressed on cell surface such as CD19, CD20, Mesothelin, PSMS or BCMA. Methods to engineer the variable domains of a TCR (e.g., CDR grafting etc.) are known in the art and can be used to generate HLA-independent TCR that can bind to proteins (e.g., CD19, MSLN, PSMA etc.) or protein epitopes expressed extracellularly independent of the MHC complex. This disclosure provides bispecific, biparatopic and multispecific SARs with the backbone of a TCR, including HLA-independent TCR, comprising one or more AABDs. The AABD domains of the SARs of the disclosure with the backbone of a TCR (e.g., HLA independent TCR) can be fully human, humanized or non-human. In an embodiment, the disclosure provides TCR (e.g., HLA independent TCR) comprising one or more fully human vH domains. In an embodiment, the disclosure provides TCR (e.g., HLA independent TCR) comprising one or more fully human vL domains.

An “HLA-independent TCR variable domain” as defined herein is the variable domain of a TCR that can bind to an antigen in an HLA-independent manner. An HLA independent variable domain may be the variable domain of an HLA independent TCRα, TCRβ, TCRγ, TCRδ or pre-TCRα. An HLA independent TCR variable domain may be a single variable domain TCR (i.e., svd-TCR). An HLA independent TCR variable domain may be a naturally occurring HLA-independent variable domain or an engineered HLA-independent variable domain. In an exemplary embodiment, an engineered HLA-independent variable domain can be generated against the extracellular domain of a protein (e.g., CD19, CD22, BCMA, MSLN, PSMA) using techniques known in the art (e.g., CDR grafting, screening phage display libraries etc.).

As used herein, “HLA-restricted” or “MHC-restricted” refers to antigen recognition requiring both MHC molecule and its peptide. Unlike antigen recognition that is “not HLA-restricted” or “HLA-independent” or “not MHC-restricted.”

As used herein, the term “heterologous gene” refers to a gene that is not in its natural environment. For example, a heterologous gene includes a gene from one species introduced into another species. A heterologous gene also includes a gene native to an organism that has been altered in some way (e.g., mutated, added in multiple copies, linked to non-native regulatory sequences, etc.). As another example, a heterologous gene includes a gene expressed in a previous or future cell lineage or differentiation state of a cell. Heterologous genes are distinguished from endogenous genes in that the heterologous gene sequences are typically joined to DNA sequences that are not found naturally associated with the gene sequences in the chromosome or are associated with portions of the chromosome not found in nature (e.g., genes expressed in loci where the gene is not normally expressed).

“Hinge region” (HR) as used herein refers to the hydrophilic region which is between the antigen binding domain and the transmembrane domain of a SAR. The hinge regions include but are not limited to Fc fragments of antibodies or fragments or derivatives thereof, hinge regions of antibodies or fragments or derivatives thereof, CH2 regions of antibodies, CH3 regions of antibodies, artificial spacer sequences or combinations thereof. Examples of hinge regions include but are not limited to CD8a hinge, and artificial spacers made of polypeptides which may be as small as, for example, Gly3 or CH1 and CH3 domains of IgGs (such as human IgG4). In some embodiments, the hinge region is any one or more of (i) a hinge, CH2 and CH3 regions of IgG4, (ii) a hinge region of IgG4, (iii) a hinge and CH2 of IgG4, (iv) a hinge region of CD8a, (v) a hinge, CH2 and CH3 regions of IgG1, (vi) a hinge region of IgG1 or (vi) a hinge and CH2 region of IgG1. Several exemplary hinge regions are provided in Table 29 of the disclosure. Other hinge regions will be apparent to those of skill in the art and may be used in connection with alternate embodiments of the disclosure.

The term “immune disorder” refers to a disease characterized by dysfunction of immune system. An autoimmune disease is a condition arising from an abnormal immune response to a normal body part. There are at least 80 types of autoimmune diseases.

“Immune effector cell,” as that term is used herein, refers to a cell that is involved in an immune response, e.g., in the promotion of an immune effector response. Examples of immune effector cells include T cells, e.g., alpha/beta T cells and gamma/delta T cells, B cells, natural killer (NK) cells, natural killer T (NKT) cells, mast cells, monocytes/macrophages and myeloid-derived phagocytes.

“Immune effector function” or “immune effector response,” “effector function” refers to the specialized function of a differentiated cell. Effector function of a T-cell or NK-cells, for example, may be cytolytic activity or helper activity including the secretion of cytokines. For example, an immune effector function or response refers a property of a T or NK cell that promotes killing or the inhibition of growth or proliferation, of a target cell. In the case of a T cell, primary stimulation and co-stimulation are examples of immune effector function or response. In case of antigen presenting cells (e.g., dendritic cells) antigen presentation and cytokine secretion are examples of effector functions.

“Immune response” as used herein refers to immunities including but not limited to innate immunity, humoral immunity, cellular immunity, immunity, inflammatory response, acquired (adaptive) immunity, autoimmunity and/or overactive immunity.

As used herein “Interleukin-2” (“IL-2”) and “Interleukin-15” (“IL-15”) refer to cytokines that regulates T and NK cell activation and proliferation. These cytokines share many biological activities. They are found to bind common receptor subunits, and may compete for the same receptor, and thus negatively regulate each other's activity. The sequence of a variety of IL-2 and IL-15 molecules are known in the art. In one aspect, the IL-2 is a wild type IL-2 or its variants with 70-99.9% amino acid sequence homology (e.g., SEQ ID NO: 7833-7837). In one aspect, the IL-15 is a wild type IL-15 or its variants with 70-99.9% amino acid sequence homology (e.g., SEQ ID NO: 7838-7841). In some aspects, IL-2 is a mammalian IL-2. In some aspects, the IL-15 is a mammalian IL-15 (e.g., Homo sapiens interleukin 15 (IL15), transcript variant 3, mRNA, NCBI Reference Sequence: NM_000585.4; Canis lupus familiaris interleukin 15 (IL15), mRNA, NCBI Reference Sequence: NM_001197188.1; Felis catus interleukin 15 (IL15), mRNA, NCBI Reference Sequence: NM_001009207.1). In particular aspects, all or a functional portion of the IL-2 or IL-15 are linked to all or a portion of a transmembrane protein. In one aspect, the NK cell or T cell expresses a fusion protein comprising all or a portion of IL-2 or IL-15 fused to all or a portion of a transmembrane protein. In a particular aspect, the portion of the transmembrane protein comprises all or a portion of a transmembrane domain of the transmembrane protein.

An “intracellular signaling domain,” (ISD) or “activation domain” as the term is used herein, refers to an intracellular signaling portion of a molecule. The intracellular signaling domain generates a signal that promotes an immune effector function of the cell. Examples of immune effector function include cytolytic activity and helper activity, including the secretion of cytokines. Examples of domains that transduce the effector function signal include but are not limited to the z chain of the T-cell receptor complex or any of its homologs, human CD3 zeta chain, CD3 polypeptides (γ, δ and ε), syk family tyrosine kinases (Syk, ZAP 70, etc.), src family tyrosine kinases (Lck, Fyn, Lyn, etc.) and other molecules involved in T-cell transduction, such as CD2, CD5 and CD28. Other intracellular signaling domains will be apparent to those of skill in the art and may be used in connection with alternate embodiments of the disclosure.

In another embodiment, the intracellular signaling domain can comprise a “primary intracellular signaling domain” or an “activation domain”. Exemplary primary intracellular signaling domains include those derived from the molecules responsible for primary stimulation, or antigen dependent simulation. In another embodiment, the intracellular signaling domain can comprise a costimulatory intracellular domain. Exemplary costimulatory intracellular signaling domains include those derived from molecules responsible for costimulatory signals, or antigen independent stimulation. For example, a primary intracellular signaling domain can comprise a cytoplasmic sequence of CD3z, and a costimulatory intracellular signaling domain can comprise cytoplasmic sequence from co-receptor or costimulatory molecule, such as CD28 or 41BB.

A primary intracellular signaling domain can comprise a signaling motif which is known as an immunoreceptor tyrosine-based activation motif or ITAM. Examples of ITAM containing primary cytoplasmic signaling sequences include, but are not limited to, those derived from CD3-zeta, common FcR gamma (FCER1G or FcRγ or FCRG), Fc gamma RIIIa, FcR beta (Fc Epsilon Rib), CD3 gamma, CD3 delta, CD3 epsilon, CD79a, CD79b, DAP10, and DAP12.

The term “isolated” as used herein refers to molecules or biologicals or cellular materials being substantially free from other materials. In one aspect, the term “isolated” refers to nucleic acid, such as DNA or RNA, or protein or polypeptide (e.g., an antibody or derivative thereof), or cell or cellular organelle, or tissue or organ, separated from other DNAs or RNAs, or proteins or polypeptides, or cells or cellular organelles, or tissues or organs, respectively, that are present in the natural source. The term “isolated” also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides. The term “isolated” is also used herein to refer to cells or tissues that are isolated from other cells or tissues and is meant to encompass both, cultured and engineered cells or tissues.

A “long linker” or “long linker domain” is a linker that is between 25 to 500 amino acids in length. In an embodiment, a long linker is about 25, 30, 35, 40, 45, 50, 55, 60, 65, 70, 75, 80, 85, 90, 95,100,105, 110, 115, 120, 125, 130, 135, 140, 145, 150, 160, 170, 180, 190, 200, 210, 220, 230, 250, 275, 300, 325, 350, 375, 400, 450, 500 amino acids and any number in between in length. In an embodiment, a long linker is between 25 and 125 amino acids in length. In an embodiment, a long linker is between 50 and 150 amino acids in length. In an embodiment, a long linker is between 75 and 175 amino acids in length. In an embodiment, a long linker is between 100 and 200 amino acids in length. In an embodiment, a long linker is between 120 and 220 amino acids in length. In an embodiment, a long linker is between 100 and 300 amino acids in length.

In an embodiment, the linker encodes for or comprises of an immunoglobulin (Ig) domain or an Ig like domain or a fragment thereof. The terms “Ig domain”, “Ig linker domain”, “Ig like domains” or “Ig like linker domains” are used interchangeably in this disclosure. The immunoglobulin domain is a type of protein domain that consists of a 2-layer sandwich of 7-9 antiparallel P-strands arranged in two P-sheets with a Greek key topology, consisting of about 125 amino acids. The Ig domains can be classified as IgV, IgC1, IgC2, or IgI. IgV domains with 9 beta strands are generally longer than IgC domains with 7 beta strands. In an embodiment, the linker comprises an IgV domain or a fragment thereof. In an embodiment, the linker comprises an IgC domain or a fragment thereof. Ig domains are found in immunoglobulins, T cell receptor chains, class I MHC, class II MHC, 02 microglobulin, coreceptors (e.g., CD4, CD8, CD19 etc.), antigen receptor accessory molecules (e.g., CD3y, CD36, CD3R, CD79a, CD79b), costimulatory or inhibitory molecules (e.g., CD28, CD80, CD86), NK cell receptors (e.g., KIR), Leukocyte immunologlobulin like receptor (LILR), IgSF CAMs (e.g., NCAM, ICAM, CD2 etc.), cytokine receptors (e.g., IL-1R, CSF-1R etc.), growth factor receptors (e.g., PDGFR), Receptor tyrosine kinases and phosphatases, Ig binding receptors, cytoskeleton proteins (e.g., titin, palladin etc.) and other proteins (e.g., CD147, CD90 etc.). Exemplary Ig linker domains are IgCL (SEQ ID NO:3536) and IgG1-CH1 (SEQ ID NO: 3537). Additional exemplary Ig linkers are presented in Table 13 (SEQ ID NO (PRT): 3538-3569). In an embodiment, the linker possesses an E set domain. An E set domain is an “Early” Ig-like fold families possibly related to the immunoglobulin and/or fibronectin type III superfamilies. In an embodiment, the linker possesses a Fibronectin type III domain.

In some embodiments, the SAR of the disclosure comprises an Fv-like or Fc-TCR antigen-binding module comprising a) a first polypeptide chain comprising a first antigen-binding domain comprising a vL, Vα or Vγ domain and b) a second polypeptide chain comprising a second antigen-binding domain comprising a vH, Vβ or Vδ domain. In some embodiments, there is a first peptide linker fused to the C-terminus of the vL, Vα or Vγ domain and/or a second peptide linker fused to the C-terminus of the vL, Vα or Vγ domain. In some embodiments, the first and second peptide linkers are capable of binding to one another. In some embodiments, the first and/or second peptide linkers are derived from immunoglobulin heavy and/or light chain constant regions. In some embodiments, the first and/or second peptide linkers comprise a CH3 antibody domain or a variant thereof. In some embodiments, immunoglobulin heavy chain constant domains (e.g., CHI or CH3) contained in the peptide linkers are derived from an IgG (e.g., IgG1, IgG2, IgG3, or IgG4), IgA (e.g., IgA1 or IgA2), IgD, IgM, or IgE heavy chain, optionally human. In some embodiments, the first and/or second peptide linkers are derived from TCR subunit constant regions. For example, in some embodiments, the first and/or second peptide linkers are derived from a) TCR α and β subunit constant domains; or b) TCR γ and δ subunit constant domains. In some embodiments, the first and/or second peptide linkers are synthetic. In some embodiments, all of the vL, Vα or Vγ and vH, Vβ or Vδ CDRs are derived from the same antibody or TCR moiety. In some embodiments, the vL antibody domain and the vH antibody domain comprise antibody CDRs derived from more than one antibody moiety. In some embodiments, the vL antibody domain comprises antibody CDRs derived from a vH antibody domain and/or the vL antibody domain comprises antibody CDRs derived from a vH antibody domain. In some embodiments, the vL antibody domain comprises framework regions derived from one antibody and one or more CDRs derived from another antibody and/or the vH antibody domain comprises framework regions derived from one antibody and one or more CDRs derived from another antibody. In some embodiments, the Vα domain and the Vβ domain comprise TCR CDRs derived from more than one TCR. In some embodiments, the Vα domain comprises CDRs derived from a Vβ TCR domain and/or the Vβ domain comprises CDRs derived from Vα domain. In some embodiments, the Vα domain comprises framework regions derived from one TCR and one or more CDRs derived from another TCR and/or the Vβ domain comprises framework regions derived from one TCR and one or more CDRs derived from another TCR. In some embodiments, the Vγ domain and the Vδ domain comprise TCR CDRs derived from more than one TCR. In some embodiments, the Vγ domain comprises CDRs derived from a Vδ TCR domain and/or the Vδ domain comprises CDRs derived from Vγ domain. In some embodiments, the Vγ domain comprises framework regions derived from one TCR and one or more CDRs derived from another TCR and/or the Vδ domain comprises framework regions derived from one TCR and one or more CDRs derived from another TCR. In some embodiments, the first and second polypeptide chains are linked, such as by a covalent linkage (e.g., peptide or other chemical linkage) or non-covalent linkage. In some embodiments, the first and second antigen-binding domains are linked by a disulfide bond. In some embodiments, the first and second peptide linkers are linked by a disulfide bond. In some embodiments, the first and/or second peptide linker is a variant comprising one or more modifications (e.g., amino acid substitutions, insertions, and/or deletions) compared to the sequence from which it is derived. In some embodiments, the first and/or second peptide linkers comprise one or more modifications that do not substantially alter their binding affinity for one another. In some embodiments, the first and/or second peptide linkers comprise one or more modifications that increase their binding affinity for one another and/or introduce a non-naturally occurring disulfide bond. In some embodiments, the first and second peptide linkers comprise a knob-into-hole modification (see, for example, Carter P. Immunol Methods. 248:7-15, 2001). In some embodiments, the first and second peptide linkers are modified by electrostatic steering to enhance their association with one another (see, for example, WO2006106905 and Gunasekaran K, et al. J Biol Chem. 285: 19637-46, 2010). In some embodiments, the Fv-like or TCR-Fv-like antigen-binding module is human, humanized, chimeric, semi-synthetic, or fully synthetic.

An exemplary SAR construct comprising IgCL and IgG1-CH1 linkers is represented by CD8SP-hu-mROO5-1-vL-xho-IgCL-Bam-DAP10-opt1-Spe-CD3zCP-opt1-F-P2A-dSPE-IgSP-hu-mROO5-1-vH-Mlu-IgG1-CH1-Kpn-DAP10-opt2-Xba-CD3zCP-opt2-F-F2A-dXBA-Nde-K13-opt (SEQ ID NO: 5869). The IgG1-CH1 inker (SEQ ID NO (DNA): 1143, SEQ ID NO (PRT): 3537) in this construct can be replaced by other Ig like linkers shown in Table 13 such as IgG2-IC-CHI1, IgG3-CHI1, IgG4-CHI1, IgAI-CHI1, IgA2-CHI1, IgD-CHI1, IgE-CHI1 or IgM-CHI1. The IgCL and IgG1-CH1 linkers can be also replaced by the Ig like linkers derived from TCRα and TCRβ, respectively (Table 13). Alternatively, the IgCL and IgG1-CH1 linkers can be also replaced by the Ig like linkers derived from TCRγ and TCRδ chains (Table 13).

As used herein, the term “ligand” refers to a molecule that binds to a receptor. In particular, the ligand binds a receptor on another cell, allowing for cell-to-cell recognition and/or interaction.

As used herein, the term “linker” (also “linker domain” or “linker region”) refers to an oligo or a polypeptide (or an oligo encoding the polypeptide) that joins together two or more domains or regions of a SAR polynucleotide or polypeptide, respectively, disclosed herein. The linker can be anywhere from 1 to 500 amino acids in length or 3 to 1500 nucleotide in length. In some embodiments the “linker” is cleavable or non-cleavable. Unless specified otherwise, the term “linker” used herein means a non-cleavable linker. Said non-cleavable linkers may be composed of flexible residues which allow freedom of motion of adjacent protein domains relative to one another. Non-limiting examples of such residues include glycine and serine. In some embodiments, linkers include non-flexible residues. Examples of cleavable linkers include 2A linkers (for example T2A), 2A-like linkers or functional equivalents thereof and combinations thereof. In some embodiments, the linkers include the picornaviral 2A-like linker, CHYSEL sequences of porcine teschovirus (P2A), Thosea asigna virus (T2A) or combinations, variants and functional equivalents thereof. In some embodiments, the linker sequences may comprise a motif that results in cleavage between the 2A glycine and the 2B proline (see, e.g., T2A sequence). The nucleic sequences of several exemplary cleavable linkers are provided in SEQ ID NO: 1233 to SEQ ID NO: 1238 and amino acid sequences of several exemplary linkers are provided in SEQ ID NO: 3627 to SEQ ID NO: 3632. Other cleavable linkers that may be used herein are readily appreciated by those of skill in the art. Linker modules also refer to TCR and Antibody linkers presented in Table 13.

In an embodiment, a Ser-Gly-Ser-Gly (SGSG) motif (SEQ ID NOs: 3633) is also added upstream of the cleavable linker sequences to enhance the efficiency of cleavage. A potential drawback of the cleavable linkers is the possibility that the small 2A tag left at the end of the N-terminal protein may affect protein function or contribute to the antigenicity of the proteins. To overcome this limitation, in some embodiments, a furine cleavage site (RAKR) (SEQ ID NO: 3635) is added upstream of the SGSG motifs to facilitate cleavage of the residual 2A peptide following translation.

The term “lentivirus” refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lenti viruses.

The term “lentiviral vector” refers to a vector derived from at least a portion of a lentivirus genome, including especially a self-inactivating lentiviral vector as provided in Milone et al., Mol. Ther. 17(8): 1453-1464 (2009). Other examples of lentivirus vectors that may be used in the clinic include but are not limited to, e.g., the LENTIVECTOR® gene delivery technology from Oxford BioMedica, the LENTIMAX™ vector system from Lentigen and the like. Nonclinical types of lentiviral vectors are also available and would be known to one skilled in the art. Other examples of lentivirus vectors are pLENTI-EF1α (SEQ ID NO: 1), pLENTI-EF1α-DWPRE (SEQ ID NO: 2), pCCLc-MNDU3-WPRE (SEQ ID NO: 4) and pCCLc-MNDU3-Eco-Nhe-Sal-WPRE (SEQ ID NO: 5). In an exemplary embodiment, the nucleic acid fragment encoding a SAR, or SAR plus accessory module(s), or the accessory module(s) can be cloned between the Nhe I and Sal I sites present in the pLENTI-EF1α and the pCCLc-MNDU3-Eco-Nhe-Sal-WPRE vectors using methods known in the art.

“Killer cell immunoglobulin-like receptors” or “KIRs” as used herein refer to a family of transmembrane glycoproteins expressed by natural killer cells and subsets of T cells.

“Mammal” as used herein refers to any member of the class Mammalia.

A “marker gene” encodes for a protein not normally expressed by the target cell which allows for identification of successful transduction. A marker gene can be also used for selective depletion or enrichment of transduced cells (e.g., SAR-expressing cells). Exemplary marker genes include tEGFR, CD20, tCD19, tBCMA and RQR8.

A “multipurpose switch” or “multipurpose gene” encodes for a protein that provide suicide, survival and marker functions. In an embodiment, all the above functions are provided by a single polypeptide chain. Exemplary multipurpose switches include IL2-tBCMA, IL15-tBCMA, IL2-RQR8, and IL2-tHer2 etc.

“Mimotope” as used herein is a macromolecule, often a peptide, which mimics the structure of an epitope. Because of this property it causes an antibody response similar to the one elicited by the epitope. An antibody for a given epitope antigen will recognize a mimotope which mimics that epitope. Mimotopes are a kind of peptide aptamers.

The term “multi-chain synthetic antigen receptor” “multi-chain SAR” means a synthetic antigen receptor comprising two or more polypeptide chains. A multi-chain SAR can be a double chain SAR. A double chain SAR comprises two membrane associated domain (e.g., transmembrane or membrane anchoring domains). An exemplary multi-chain SAR targeting CD19 is CD8SP-CD19-hu-mROO5-1-vL-Xho-CD16-F158V-FL-TMCP-v1-F-P2A-Spe-SP-Bst-CD19-hu-mROO5-1-vH-Mlu-CD16-F158V-S197P-FL-TMCP-v3-F-F2A-Xba-PAC (SEQ ID NO (DNA): 5451 and SEQ ID NO (PRT): 6283). In this SAR construct, the hu-mROO5-1 vL fragment is operationally linked to CD16-F158V-FL-TMCP-v1 module and the hu-mROO5-1-vH fragment is operationally linked to the CD16-F158V-S197P-FL-TMCP-v3. The two chains of this SAR are separated by Furine (F) and P2A cleavable linker sequences. This SAR construct also expresses a puromycin resistance gene (PAC) that is separated from the SAR polypeptide by a Furine (F) and F2A cleavable linker sequences. As SAR are modular in design, the CD16A-F158V-S197P-FL-v3 module and CD16-F158V-FL-TMCP-v1 modules can be replaced by other signaling modules to generate SAR with different signaling chains. Further the hu-mROO5-1 vL and hu-mROO5-vH fragments can be replaced by antigen binding domains (e.g., vL, vH, vHH, FHVH, centyrin, svd-TCR etc.) targeting other antigens to generate SAR targeting different antigens. Exemplary such multi-chain SARs are provided in Table 41 of provisional application. (e.g., SEQ ID NO: 5451-5462, 5483-5494, 5515-5526, 5547-5558, 5579-5590, 5611-5622, 5643-5654 etc.). The expression and activity of these novel SARs can be tested using methods described in the disclosure to select the SARs with optimal functional activities.

As used herein, “MHC” or “major histocompatibility complex” refers to cell surface molecules encoded by a large number of genes in mammals. MHC molecules include Class I and Class II. Class I molecules are alternatively referred to in humans as “HLA” or “human leukocyte antigen.” In part due to the complexity of HLA molecule expression HLA may also be referred to as an HLA system. Humans express HLA-A, HLA-B and HLA-C molecules that are typically involved with presenting processed antigen to CD8 cells, i.e., HLA restricted. Class II molecules, such as DR, DQ, DP, etc., are typically involved with presenting externally derived peptides to CD4+ cells, i.e., MHC Class II restricted. MHC restricted in general encompasses both Class I and Class II as in transplantation (bone marrow) matching.

“Native” or “Naturally occurring” or “endogenous” as used herein refers to a gene, protein, nucleic acid (e.g., DNA, RNA etc.) or fragment thereof that is native to a cell or is naturally expressed in a cell. Thus, a native or endogenous TCRα chain polypeptide of a T cell consists of a variable domain (Vu) joined to a TCRα constant chain. The native or endogenous TCRα chain precursor polypeptide also consists of an amino-terminal signal peptide that is cleaved from the mature polypeptide.

“Native receptor” or “Naturally occurring receptor” or “endogenous receptor” or “native receptor” as used herein refers to any receptor that occurs in nature and comprises an antigen binding or a ligand binding domain. The term includes functional variants, isoforms and homologs from other mammalian species. A native receptor can be “native signaling receptor” or a “naturally occurring signaling receptor” if it is capable of transmitting a cell signal upon binding to its target. A naturally occurring receptor or native receptor is native to a cell or is naturally expressed in a cell. Examples of naturally occurring signaling receptors or native receptors include, but are not limited to, CD16A, CD16B, NKp30, NKp44, NKp46, KIR2DS4, NKG2D etc. For the purpose of this disclosure, the CD3 signaling chains (CD3ε, CD3γ, CD3δ and CD3ƒ) are not included within the definition of a “naturally occurring receptor” and are instead classified as a signaling adaptor.

As used herein, the term “non-TCR naturally occurring receptor” or ““non-TCR naturally occurring signaling receptor” or “non-TCR receptor” or ‘non-TCR signaling receptor” refers to a receptor that is not a T cell receptor (TCR). A non-TCR receptor can be expressed in cells other than a T cell. A non-TCR receptor can be expressed in cells that lack the expression of CD3ƒ, CD38, CD3δ and/or CD3y chains. A “non-TCR naturally occurring receptor” lacks the transmembrane domain and/or cytosolic domain of TCRα, TCRβ, TCRγ, TCRδ or pre-TCRα. A “non-TCR naturally occurring receptor” does not recruit the entire TCR signaling module. In an embodiment, a “non-TCR naturally occurring receptor” does not comprise the TCRα, TCRβ, TCRγ, TCRδ or pre-TCRα polypeptides. In an embodiment, a “non-TCR naturally occurring receptor” does not comprise the entire coding region of TCRα, TCRβ, TCRγ, TCRδ or pre-TCRα. In an embodiment, a “non-TCR naturally occurring receptor” does not comprise the entire constant chains of TCRα, TCRβ, TCRγ, TCRδ or pre-TCRα. In an embodiment, a “non-TCR naturally occurring receptor” does not comprise the entire hinge domains (or connecting peptides) of TCRα, TCRβ, TCRγ, TCRδ or pre-TCRα. In an embodiment, a “non-TCR naturally occurring receptor” does not comprise the entire transmembrane domains and cytosolic domains of TCRα, TCRβ, TCRγ, TCRδ or pre-TCRα. A non-TCR receptor can be expressed in cells other than a T cell. A ‘non-TCR signaling receptor” may comprise a fragment of a TCR such as TCR variable domains (e.g., Vα, Vβ, Vγ, Vδ) or Ig domains (e.g., SEQ ID: 1158-1175). A non-TCR signaling receptor does not comprise the entire TCR constant chains (i.e., constant chains of TCRα, TCRβ, TCRγ, TCRδ or pre-TCRα).

As used herein, the term “non-T cell receptor module” or ““non-TCR module” or “non-TCR signaling module” or “NTCRM” refers to a module that lacks sequences comprised of the T cell receptor transmembrane domains and may further lack all or a portion of T cell receptor connecting peptides and/or intracellular domains. An NTCRM lacks sequences comprised of the transmembrane domains of TCRα, TCRβ, TCRγ, TCRδ or pre-TCRα. An NTCRM may further lack all or a portion of the connecting peptides and/or intracellular domains of TCRα, TCRβ, TCRγ, TCRδ or pre-TCRα.

As used herein, the term “non-CD3 adaptor module” or “non-CD3 adaptor” or “non-TCR/CD3 adaptor” or ““non-TCR/CD3 signaling adaptor” or ““NCAM” refers to a signaling adaptor that is not a component of the T cell receptor/CD3 receptor complex. In an embodiment, a “non-TCR/CD3 adaptor” does not comprise the transmembrane and/or cytosolic regions of CD3ε, CD3ƒ, CD3y or CD3δ chains or variants thereof.

The term “near the N-terminus” as used herein means within the N-terminal 30 amino acids. For example, the term “an AABD operably linked to the N-terminus or near the N-terminus of a vL and/or vH domain”, mean an AABD that is operably linked at the N-terminus of a vL or a vH fragment or operably linked to the N-terminal 2, 3, 4, 5, 6, 7, 8, 9, 10, 11, 12, 13, 14, 15, 16, 17, 18, 19, 25 or 30 amino acid comprising the vL or the vH domain. Similarly, the term “an AABD operably linked to the N-terminus or near the N-terminus of a Va and/or Vb domain”, mean an AABD that is operably linked at the N-terminus of a Va or a Vb fragment or operably linked to the N-terminal 2, 3, 4, 5, 6, 7, 8, 9, 10, 11, 12, 13, 14, 15, 16, 17, 18, 19, 20, 21, 22, 23, 24, 25 or 30 amino acid comprising the Va or the Vb domain. An AABD of the disclosure may be operably linked to or near the N-terminus of another domain either directly or via an intervening linker sequence.

As used herein, “Natural Killer Cell Receptor” or “NK receptor” refers to a cell surface receptor that is expressed in natural killer (NK) cells and includes functional variants, isoforms and homologs from other mammalian species. An NK receptor may be an activating receptor or an inhibitory receptor. Exemplary activating NK receptors include NKp30, NKp44, NKp46, NKG2D and KIR3DS4. Exemplary inhibitory NK receptors include CD94-NKG2A, TIGIT and CD96.

As used herein, “Natural Killer Cells” (“NK cells”) refer to a type of cytotoxic lymphocyte of the immune system.

As used herein “NKp30” or “NCR3” is a gene (Gene ID: 259197) that encodes for a protein that is a natural cytotoxicity receptor (NCR) that may aid NK cells in the lysis of tumor cells. The term includes functional variants, isoforms and homologs from other mammalian species.

As used herein “NKp44” or “NCR2” is a gene (Gene ID: 9436) that encodes for a protein that is a natural cytotoxicity receptor (NCR). The term includes functional variants, isoforms and homologs from other mammalian species.

As used herein “NKp46” or “NCRT” is a gene (Gene ID: 9437) that encodes for a protein that is a natural cytotoxicity receptor (NCR). Five transcript variants encoding different isoforms have been found for this gene. The term includes functional variants, isoforms and homologs from other mammalian species.

As used herein “NKG2D” or “KLRK1” is a gene (Gene ID: 22914) that encodes for a protein is a member of C-type lectins. The encoded transmembrane protein is characterized by a type II membrane orientation (has an extracellular C terminus) and the presence of a C-type lectin domain. The term includes functional variants, isoforms and homologs from other mammalian species.

As used herein a “non-naturally occurring agent” or “non-native” or “exogenous” refers to an agent that is not naturally expressed in a cell. Stated another way, the non-naturally occurring agent is “engineered” to be expressed in a cell. A non-naturally occurring agent may be a cloned version of a naturally occurring agent. Exemplary non-naturally occurring agents include SARs (e.g., CAR, SIRs, Ab-TCRs, TFPs, recombinant TCR). A non-naturally occurring agent may be expressed into a cell using techniques of gene transfer known in the art, such as lentiviral or retroviral mediated gene transfer. A non-naturally occurring agent may be expressed in an immune cell using an exogenous promoter (e.g., EF1α promoter) or an endogenous promoter (e.g., TCRα or TRAC promoter). When an endogenous gene (e.g., CD16, NKp30 etc.) is cloned and ectopically expressed in a cell, it represents another example of a non-naturally occurring agent.

As used herein a “non-naturally occurring immune receptor” or “exogenous immune receptor” “non-naturally occurring receptor” refers to an immune receptor that is not naturally expressed in an immune cell. Stated another way, the non-naturally occurring immune receptor is “engineered” to be expressed in an immune cell. A non-naturally occurring immune receptor may be a cloned version of a naturally occurring immune receptor. Alternatively, a non-naturally occurring immune receptor may be a chimeric receptor that is produced using recombinant molecular biology techniques. An exemplary non-naturally occurring immune receptors is a SAR (e.g., 2^{nd} generation CAR, SIR, cTCR, STAR, zSIR, Ab-TCRs, TFPs and recombinant TCR).

As used herein a “non-naturally occurring TCR antigen binding domain” or “exogenous TCR antigen binding domain” refers to a binding domain operably linked to a TCR constant region that is chimeric and non-naturally occurring with respect to a TCR present in nature. Stated another way, the non-naturally occurring TCR antigen binding domain is “engineered” using recombinant molecular biology techniques to be operably linked to a TCR and moreover, that the antigen binding domain is obtain or derived from a molecule that is distinct from a TCR found in nature. An antigen binding domain that is distinct from a TCR in nature includes antibody vH and vL fragments, humanized antibody fragments, chimeric antibody fragments, receptor ligands, and the like.

As used herein a “non-naturally occurring antigen binding domain” or “non-naturally occurring extracellular antigen binding domain” or “heterologous antigen binding domain” refers to an antigen binding domain that is not part of a naturally occurring receptor. Stated another way, the non-naturally occurring antigen binding domain is “engineered” using recombinant molecular biology techniques to be operably linked to a naturally occurring signaling receptor and moreover, that the antigen binding domain is obtained or derived from a molecule that is distinct from a signaling receptor found in nature. Exemplary heterologous antigen binding domains include antibodies, antibody fragments (e.g., vL, vH, scFv, Fab, F(ab)2 etc.), single domain antibodies (e.g., sVH, FHVH, vHH etc.), non-immunoglobulin antigen binding domains, single variable domain-TCR (svd-TCR), recombinant TCRs, HLA-independent TCR, scTCR, epitopes, adaptors, ligands and receptors.

The term “operably linked” or “functionally linked” or “operationally linked” refers to functional linkage or association between a first component and a second component such that each component can be functional. For example, operably linked includes the association between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. In the context of two polypeptides that are operably linked a first polypeptide functions in the manner it would independent of any linkage and the second polypeptide functions as it would absent a linkage between the two. The term “operationally linked” when used in the context of different domains of SARs of the disclosure refers to domains that are linked via a covalent bond (e.g., a peptide bond or a non-peptide chemical bond). In an exemplary embodiment, a SAR comprising a heterologous antigen binding domain (e.g., a CD19 scFv) that is operationally linked to the N-terminus of the extracellular domain of CD16A refers to a SAR polypeptide that is encoded by a nucleic acid sequence comprising a CD19 scFv that is fused in frame to the nucleic acid sequence encoding the extracellular, transmembrane and cytosolic domains of CD16A. In an embodiment, the operational linkage between the different domains of a SAR polypeptide is achieved via a peptide bond. However, in certain embodiments, the different domains of a SAR can be linked via non-peptide bonds, e.g., a disulfide bond or via chemical conjugation etc.

“Percent identity” in the context of two or more nucleic acids or polypeptide sequences, refers to two or more sequences that are the same. Two sequences are “substantially identical” if two sequences have a specified percentage of amino acid residues or nucleotides that are the same (e.g., 60% identity, optionally 70%, 71%, 72%, 73%, 74%, 75%, 76%, 77%, 78%7, 79%, 80%,81%, 82%, 83%, 84%, 85%, 86%, 87%, 88%, 89%, 90%, 91%, 92%, 93%, 94%, 95%, 96%, 97%, 98%, 99% identity over a specified region, or, when not specified, over the entire sequence), when compared and aligned for maximum correspondence over a comparison window, or designated region as measured using one of the following sequence comparison algorithms or by manual alignment and visual inspection. Optionally, the identity exists over a region that is at least about 50 nucleotides (or 10 amino acids) in length, or more typically over a region that is 100 to 500 or 1000 or more nucleotides (or 20, 50, 200 or more amino acids) in length.

Two examples of algorithms that can be used for determining percent sequence identity and sequence similarity are the BLAST and BLAST 2.0 algorithms, which are described in Altschul et al., (1977) Nuc. Acids Res. 25:3389-3402; and Altschul et al., (1990) J. Mol. Biol. 215:403-410, respectively. Software for performing BLAST analyses is publicly available through the National Center for Biotechnology Information.

The percent identity between two amino acid sequences can also be determined using the algorithm of E. Meyers and W. Miller, (1988) Comput. Appl. Biosci. 4:11-17) which has been incorporated into the ALIGN program (version 2.0), using a PAM120 weight residue table, a gap length penalty of 12 and a gap penalty of 4.

Non-limiting examples of target antigens are listed in Table B. A SAR of the disclosure may bind one or more (e.g., 2, 3, 4, 5 or more) target antigens listed in Table B either directly or via SAR adaptors described herein.

TABLE B
TABLE B: Exemplary Antigens Targeted by Antibodies, antibody
fragments (e.g., scFv), AABD (e.g., FHVH, vHH, DARPIN, Centryin,
D domains, Adaptors etc.) and SARS of the disclosure

CD5; CD19; CD123; CD22; CD30; CD171; CS1 (also referred to as CD2 subset 1,
CRACC, SLAMF7, CD319, and 19A24); C-type lectin-like molecule-1 (CLL-1 or
CLECL1); CD33; epidermal growth factor receptor variant III (EGFRviii); ganglioside G2
(GD2); ganglioside GD3 (aNeu5Ac(2-8)aNeu5Ac(2-3)bDGalp(l-4)bDGlcp(l-l)Cer); TNF
receptor family member B cell maturation (BCMA); Tn antigen ((Tn Ag) or (GalNAcα-
Ser/Thr)); prostate-specific membrane antigen (PSMA); Receptor tyrosine kinase-like
orphan receptor 1 (ROR1); Fms Like Tyrosine Kinase 3 (FLT3); Tumor-associated
glycoprotein 72 (TAG72); CD38; CD44v6; a glycosylated CD43 epitope expressed on
acute leukemia or lymphoma but not on hematopoietic progenitors, a glycosylated CD43
epitope expressed on non-hematopoietic cancers, Carcinoembryonic antigen (CEA);
Epithelial cell adhesion molecule (EPCAM); B7H3 (CD276); KIT (CD117); Interleukin-13
receptor subunit alpha-2 (IL-13Ra2 or CD213A2); Mesothelin; Interleukin 11 receptor
alpha (IL-llRa); prostate stem cell antigen (PSCA); Protease Serine 21 (Testisin or
PRSS21); vascular endothelial growth factor receptor 2 (VEGFR2); Lewis(Y) antigen;
CD24; Platelet-derived growth factor receptor beta (PDGFR-beta); Stage-specific
embryonic antigen-4 (SSEA-4); CD20; Folate receptor alpha (FRa or FR1); Folate receptor
beta (FRb); Receptor tyrosine-protein kinase ERBB2 (Her2/neu); Mucin 1, cell surface
associated (MUC1); epidermal growth factor receptor (EGFR); neural cell adhesion
molecule (NCAM); Prostase; prostatic acid phosphatase (PAP); elongation factor 2
mutated (ELF2M); Ephrin B2; fibroblast activation protein alpha (FAP); insulin-like
growth factor 1 receptor (IGF-I receptor), carbonic anhydrase IX (CAlX); Proteasome
(Prosome, Macropain) Subunit, Beta Type, 9 (LMP2); glycoprotein 100 (gpl00); oncogene
fusion protein consisting of breakpoint cluster region (BCR) and Abelson murine leukemia
viral oncogene homolog 1 (Abl) (bcr-abl); tyrosinase; ephrin type-A receptor 2 (EphA2);
sialyl Lewis adhesion molecule (sLe); ganglioside GM3 (aNeu5Ac(2-3)bDClalp(l-
4)bDGlcp(l-1)Cer); transglutaminase 5 (TGS5); high molecular weight-melanoma
associated antigen (HMWMAA); o-acetyl-GD2 ganglioside (OAcGD2); tumor endothelial
marker 1 (TEM1/CD248); tumor endothelial marker 7-related (TEM7R); claudin 6
(CLDN6); thyroid stimulating hormone receptor (TSHR); G protein coupled receptor class
C group 5, member D (GPRC5D); chromosome X open reading frame 61 (CXORF61);
CD97; CD179a; anaplastic lymphoma kinase (ALK); Polysialic acid; placenta-specific 1
(PLAC1); hexasaccharide portion of globoH glycoceramide (GloboH); mammary gland
differentiation antigen (NY-BR-1); uroplakin 2 (UPK2); Hepatitis A virus cellular receptor
1 (HAVCR1); adrenoceptor beta 3 (ADRB3); pannexin 3 (PANX3); G protein-coupled
receptor 20 (GPR20); lymphocyte antigen 6 complex, locus K 9 (LY6K); Olfactory
receptor 51E2 (OR51E2); TCR Gamma Alternate Reading Frame Protein (TARP); Wilms
tumor protein (WT1); Cancer/testis antigen 1 (NY-ESO-1); Cancer/testis antigen 2 (LAGE-
1a); Melanoma-associated antigen 1 (MAGE-A1); ETS translocation-variant gene 6,
located on chromosome 12p (ETV6-AML); sperm protein 17 (SPA17); X Antigen Family,
Member lA (XAGEl); angiopoietin-binding cell surface receptor 2 (Tie 2); melanoma
cancer testis antigen-1 (MAD-CT-1); melanoma cancer testis antigen-2 (MAD-CT-2); Fos-
related antigen 1; tumor protein p53 (p53); p53 mutant; prostein; survivin; telomerase;
prostate carcinoma tumor antigen-1 (PCT A-1 or Galectin 8), melanoma antigen
recognized by T cells 1 (MelanA or MARTI); Rat sarcoma (Ras) mutant; human
Telomerase reverse transcriptase (hTERT); sarcoma translocation breakpoints; melanoma
inhibitor of apoptosis (ML-IAP); ERG (transmembrane protease, serine 2 (TMPRSS2) ETS
fusion gene); N-Acetyl glucosaminyl-transferase V (NA17); paired box protein Pax-3
(PAX3); Androgen receptor; Cyclin Bl; v-myc avian myelocytomatosis viral oncogene
neuroblastoma derived homolog (MYCN); Ras Homolog Family Member C (RhoC);
Tyrosinase-related protein 2 (TRP-2); Cytochrome P450 lB 1 (CYPlB 1); CCCTC-Binding
Factor (Zinc Finger Protein)-Like (BORIS or Brother of the Regulator oflmprinted Sites),
Squamous Cell Carcinoma Antigen Recognized By T Cells 3 (SART3); Paired box protein
Pax-5 (PAX5); proacrosin binding protein sp32 (OY-TESl); lymphocyte-specific protein
tyrosine kinase (LCK); A kinase anchor protein 4 (AKAP-4); synovial sarcoma, X
breakpoint 2 (SSX2); Receptor for Advanced Glycation Endproducts (RAGE-1); renal
ubiquitous 1 (RUl); renal ubiquitous 2 (RU2); legumain; human papilloma virus E6 (HPV
E6); human papilloma virus E7 (HPV E7); intestinal carboxyl esterase; heat shock protein
70-2 mutated (mut hsp70-2); CD79a; CD79b; CD72; Leukocyte-associated
immunoglobulin-like receptor 1 (LAIRl); Fc fragment of IgA receptor (FCAR or CD89);
Leukocyte immunoglobulin-like receptor subfamily A member 2 (LILRA2); CD300
molecule-like family member f (CD300LF); C-type lectin domain family 12 member A
(CLEC12A); bone marrow stromal cell antigen 2 (BST2); EGF-like module-containing
mucin-like hormone receptor-like 2 (EMR2); lymphocyte antigen 75 (LY75); Glypican-3
(GPC3); Fc receptor-like 5 (FCRL5); and immunoglobulin lambda-like polypeptide 1
(IGLLl), MPL, Biotin, c-MYC epitope Tag, CD34, LAMP1 TROP2, GFRalpha4, CDH17,
CDH6, NYBR1, CDH19, CD200R, Slea (CA19.9; Sialyl Lewis Antigen); Fucosyl-GM1,
PTK7, gpNMB, CDH1-CD324, DLL3, CD276/B7H3, IL11Ra, IL13Ra2, CD179b-IGLl1,
TCRgamma-delta, NKG2D, CD32 (FCGR2A), Tn ag, Tim1-/HVCR1, CSF2RA (GM-
CSFR-alpha), TGFbetaR2, Lews Ag, TCR-betal chain, TCR-beta2 chain, TCR-gamma
chain, TCR-delta chain, FITC, Leutenizing hormone receptor (LHR), Follicle stimulating
hormone receptor (FSHR), Gonadotropin Hormone receptor (CGHR or GR), CCR4, GD3,
SLAMF6, SLAMF4, HIV1 envelope glycoprotein, HTLV1-Tax, CMV pp65, EBV-
EBNA3c, KSHV K8.1, KSHV-gH, influenza A hemagglutinin (HA), GAD, PDL1,
Guanylyl cyclase C (GCC), auto antibody to desmoglein 3 (Dsg3), auto antibody to
desmoglein 1 (Dsg1), HLA, HLA-A, HLA-A2, HLA-B, HLA-C, HLA-DP, HLA-DM,
HLA-DOA, HLA-DOB, HLA-DQ, HLA-DR, HLA-G, IgE, CD99, Ras G12V, Tissue
Factor 1 (TF1), AFP, GPRC5D, Claudin18.2 (CLD18A2 or CLDN18A.2), P-glycoprotein,
STEAP1, Liv1, Nectin-4, Cripto, gpA33, BST1/CD157, low conductance chloride channel
(LCCC), TAJ/TROY, MPL (TPO-R), KIR3DL2, CD32b, CD229, Toso and BAFF-R.

In some embodiments, the SAR of the disclosure comprise one or more antigen binding domains (e.g., vL, vH, Va, Vb, Vg, Vd, Fv, TCR-Fv, svd-TCR, scTCR etc.) that specifically bind to a complex comprising a peptide derived from a disease-associated antigen (such as a tumor-associated or virally-encoded antigen; e.g., a peptide derived from NY-ESO-1, MAGE-A3, MAGE-A4, WT1, mutant Ras, HPV16-E7, EBV-LMP2A, AFP, gp100, PSA, mutant p53, HIV-1, etc.) and an MHC class I protein, wherein the MHC class I protein is HLA-A, HLA-B, HLA-C, HLA-E, HLA-F, or HLA-G. In some embodiments, the MHC class I protein is HLA-A, HLA-B, or HLA-C. In some embodiments, the MHC class I protein is HLA-A. In some embodiments, the MHC class I protein is HLA-B. In some embodiments, the MHC class I protein is HLA-C. In some embodiments, the MHC class I protein is HLA-A01, HLA-A02, HLA-A03, HLA-A09, HLA-A 10, HLA-A11, HLA-A 19, HLA-A23, HLA-A24, HLA-A25, HLA-A26, HLA-A28, HLA-A29, HLA-A30, HLA-A31, HLA-A32, HLA-A33, HLA-A34, HLA-A36, HLA-A43, HLA-A66, HLA-A68, HLA-A69, HLA-A74, or HLA-A80. In some embodiments, the MHC class I protein is HLA-A02. In some embodiments, the MHC class I protein is any one of HLA-A*02:01-555, such as HLA-A*02:01, HLA-A*02:02, HLA-A*02:03, HLA-A*02:04, HLA-A*02:05, HLA-A*02:06, HLA-A*02:07, HLA-A*02:08, HLA-A*02:09, HLA-A*02: 10, HLA-A*02: 11, HLA-A*02: 12, HLA-A*02: 13, HLA-A*02: 14, HLA-A*02: 15, HLA-A*02: 16, HLA-A*02: 17, HLA-A*02: 18, HLA-A*02: 19, HLA-A*02:20, HLA-A*02:21, HLA-A*02:22, or HLA-A*02:24. In some embodiments, the MHC class I protein is HLA-A*02:01. HLA-A*02:01 is expressed in 39-46% of all Caucasians, and therefore represents a suitable choice of MHC class I protein for use in the disclosure.

In some embodiments, the SAR of the disclosure comprise one or more antigen binding domains (e.g., vL, vH, Va, Vb, Vg, Vd, Fv, TCR-Fv, svd-TCR, scTCR etc.) that specifically bind to a complex comprising a peptide derived from a disease-associated antigen (such as a tumor-associated or virally-encoded antigen) (e.g., a peptide derived from NY-ESO-1, MAGE-A3, MAGE-A4, WT1, mutant Ras, HPV16-E7, EBV-LMP2A, AFP, gp100, PSA, mutant p53, HIV-1, etc.) and an MHC class II protein, wherein the MHC class II protein is an HLA-DP, HLA-DQ, or HLA-DR.

The SAR of the disclosure can also bind to complex comprising a peptide derived from a disease-associated antigen and an MHC class I or class II protein from other species (e.g., dog, cat, mouse, rat, cow, horse, monkey etc.)

As used herein, the term “receptor” refers to a polypeptide, or portion thereof, present on a cell membrane that selectively binds one or more ligand.

As used herein, the terms “region” or “portion” when used in reference to a nucleic acid molecule refers to a set of linked nucleotides that is less than the entire length of the molecule, such as a CD3 signaling region described herein.

The term “retrovirus vector” refers to a vector derived from at least a portion of a retrovirus genome. Examples of retrovirus vector include MSCVneo, MSCV-pac (or MSCV-puro), MSCV-hygro as available from Addgene or Clontech.

The term “SAR” or “Synthetic Antigen Receptor”, as used herein, comprises conventional CARs (e.g., 2^{nd} generation CARs comprising 41BB or CD28 costimulatory domains and CD3z activation domain) and also encompasses newer approaches to conferring antigen specificity onto cells, such as Antibody-TCR chimeric molecules or Ab-TCR (WO 2017/070608 A1 incorporated herein by reference), TCR receptor fusion proteins or TFP (WO 2016/187349 A1 incorporated herein by reference), Synthetic Immune Receptors (SIRs) (see, WO 2018/102795 A1, incorporated herein by reference), STAR (see, WO 2020/029774), HLA-independent TCR (see, WO2019157454A1), Tri-functional T cell antigen coupler (Tri-TAC or TAC) (see, WO 2015/117229 A1, incorporated herein by reference) and zSIR (see, PCT/US2019/035096, incorporated herein by reference). Bispecific and multispecific CARs have been described in PCT/US2021/022641. The term “SAR” covers CAR as well as other antigen binding receptors, including but not limited to recombinant TCR.

Typically, the term “SAR-T” is used, to refer to T-cells that have been engineered to express a Synthetic antigen receptor. Thus, T lymphocytes bearing such SARs are generally referred to as SAR-T lymphocytes. SARs can be also expressed in cells other than T cells, such as hematopoietic stem cells, induced pluripotent stem cells (iPSC), NK cells and macrophage. In some embodiment, the SAR is expressed in an immortalized cell line, such as NK92, NK92MI or a derivative thereof. The term “SAR-NK” refers to an NK cell that has been engineered to express a SAR.

Typically, the term “SAR-T” is used, to refer to T-cells (e.g., αβ T cell, γδ T cell, Treg, TIL etc.) that have been engineered to express a Synthetic antigen receptor. Thus, T lymphocytes bearing such SARs are generally referred to as SAR-T lymphocytes. SARs can be also expressed in cells other than T cells, such as hematopoietic stem cells, embryonic stem cells, induced pluripotent stem cells (iPSC), NK cells, NKT cells, monocytes, macrophage, B-cells, granulocytes, dendritic cells, cytokine induced killer cells (CIK) etc. In some embodiment, the SAR is expressed in an immortalized cell line, such as NK92, NK92MI or a derivative thereof. The term “SAR-NK” refers to an NK (natural killer) cell that has been engineered to express a SAR.

The term “Sleeping Beauty Transposon” or “Sleeping Beauty Transposon Vector” refers to a vector derived from at least a portion of a Sleeping Beauty Transposon genome.

The term “TCR constant chain” or “constant region of T cell receptor” is defined as the constant chain of TCRα/TCRα, TCRβ1/TCRb1, TCRβ2/TCRb2, TCRγ/TCRd, TCRδ/TCRd and pre-TCRα. Exemplary TCR constant chains are listed in Table 12. A TCR constant chain can be divided into several subdomains such as Ig like C1 domain (e.g., SEQ ID NO: 1168-1175; Table 13), connecting peptide (e.g., SEQ ID NO: 1177-1184; Table 14), transmembrane domain (SEQ ID NO:1187-1190; Table 15), and cytosolic domain (e.g., SEQ ID NO: 1193-1196; Table 16). The cytosolic domains of TCRα, TCRβ1/β2, TCRγ and TCRδ chains are short and generally not believed to play any significant role in their signaling activities. The disclosure also provides exemplary deletion mutants and variants of the TCR chains (Table 12). These deletion mutants and variants can be used in the construction of SAR as long as they retain one or more of the functional and biological properties of the original TCR chains, such as the ability to pair with the complementary TCR chain, the ability to assemble with the TCR/CD3 complex and the ability to transmit a T cell signal (e.g., activate NFAT pathway) when engaged by target antigen expressing cells.

The term “single chain variable region” or “scFv” refers to a fusion protein comprising at least one antibody fragment comprising a variable region of a light chain and at least one antibody fragment comprising a variable region of a heavy chain, wherein the light and heavy chain variable regions are contiguously linked, e.g., via a synthetic linker, e.g., a short flexible polypeptide linker, and capable of being expressed as a single chain polypeptide, and wherein the scFv retains the specificity of the intact antibody from which it is derived. Unless specified, as used herein an scFv may have the vL and vH variable regions in either order, e.g., with respect to the N-terminal and C-terminal ends of the polypeptide, the scFv may comprise vL-linker-vH or may comprise vH-linker-vL. In this disclosure, a scFv is also described as vL-Gly-Ser-Linker-vH. Alternatively, a scFv is also described as (vL+vH) or (vH+vL).

As use herein, the term “specifically binds” or “is specific for” refers to measurable and reproducible interactions, such as binding between a target and an antibody or antibody moiety, that is determinative of the presence of the target in the presence of a heterogeneous population of molecules, including biological molecules. In some embodiments, a SAR or an antigen binding domain that specifically binds to an antigen reacts with one or more antigenic determinants of the antigen (for example a cell surface antigen or a peptide/MHC protein complex) with a binding affinity that is at least about 10 times its binding affinity for other targets.

The term “signaling domain” refers to the functional region of a protein which transmits information within the cell to regulate cellular activity via defined signaling pathways by generating second messengers or functioning as effectors by responding to such messengers.

The term “signaling module” refers to a molecule or molecular complex comprising one or more signaling mediators or signaling adaptors that is capable of initiating a cell signal. The cell signal may include but is not limited to activation of cell signaling pathways such as NFAT, AKT, STAT or NF-κB pathways. In an exemplary embodiment, the signaling module recruits one or more proteins having a cytoplasmic immunoreceptor tyrosine-based activation motif (ITAM) that is part of a signaling complex. For example, TCR-associated signaling modules include CDγε, CDδε and CD3ƒƒ. Exemplary, signaling modules that are operational in NK cells comprise signaling adaptors such as CD3ƒ, FcRy, DAP10 and DAP12.

The term “signaling mediator” or “signaling adaptor” refers to molecule that is capable of initiating or inhibiting a cell signal when recruited by a natural or a non-natural signaling receptor. In contrast to a signaling receptor, a signaling adaptor lacks its own antigen binding domain or ligand binding domain. Exemplary signaling adaptors include CD3ƒ(CD3z), FcRγ, DAP10, DAP12, CD38, CD3y and CD36. In an embodiment, the disclosure provides a SAR in which one or more heterologous antigen binding domains are operationally linked to the hinge domain or the transmembrane domain of one or more chains of a signaling adaptor. In an embodiment, the SAR comprises a signaling adaptor that is a component of a TCR complex (e.g., CD3ƒ, CD3ε, CD3γ, CD3ε etc.). In an embodiment, the SAR comprises a signaling adaptor that interacts with TCRα, β, γ and/or δ chains of the TCR complex. In an embodiment, the SAR comprises a signaling adaptor that does not interacts with TCRα, β, γ and/or δ chains of the TCR complex. In an embodiment, the SAR comprises a signaling adaptor (e.g., CD3ƒ that has a conserved aspartic acid residue in its transmembrane domain which interacts with positive charged residues in the TCRα/β transmembrane regions. In an embodiment, the SAR comprises a signaling adaptor that lacks a conserved aspartic acid residue in its transmembrane domain. In an embodiment, the SAR comprises a signaling adaptor that is not a component of a TCR complex (e.g., DAP10). In an embodiment, the SAR comprises a signaling adaptor that activates cell signaling (e.g., CD3ƒ). In an embodiment, the SAR comprises a signaling adaptor that inhibits cell signaling. In an embodiment, the SAR comprises a signaling adaptor that possesses one or more ITAM motifs. In an embodiment, the SAR comprises a signaling adaptor that possesses two or more ITAM motifs. In an embodiment, the SAR comprises a signaling adaptor that possesses a single ITAM motif. In an embodiment, the SAR comprises a signaling adaptors that lacks ITAM motifs. In an embodiment, the SAR comprises a signaling adaptor that is a disulfide linker dimer in its native form. In an embodiment, the signaling adaptor is not a disulfide linker dimer in its native form. In an embodiment, the SAR comprises a signaling adaptor that in its native state contains an interchain disulfide bond located in its transmembrane region. In an embodiment, the SAR comprises a signaling adaptor in its native state that contains an interchain disulfide bond that is not located in its transmembrane region. In an embodiment, the SAR comprises a signaling adaptor that in its native state contains an interchain disulfide bond that is located in its extracellular region. In an embodiment, the extracellular domain of the signaling adaptor is less than 10 amino acids in length. In an embodiment, the extracellular domain of the signaling adaptor is less than 8 amino acids in length. In an embodiment, the extracellular domain of the signaling adaptor is more than 10 amino acids in length. In an embodiment, the extracellular domain of the signaling adaptor is more than 15 amino acids in length. In an embodiment, the SAR comprises a signaling adaptor that induces protein phosphorylation. In an embodiment, the SAR comprises a signaling adaptor that induces protein dephosphorylation. In an embodiment, the SAR comprises a signaling adaptor that interacts with Zap70. In an embodiment, the SAR comprises a signaling adaptor that does not interact with Zap70. In an embodiment, the two chains of a double chain SAR comprise identical signaling adaptors (e.g., CD3ƒ and CD3ƒ). In an embodiment, the two chains of a double chain SAR comprise non-identical signaling adaptors (e.g., CD3ƒ and FcRγ or CD3ƒ and DAP10 etc.).

The term “signaling chain” or “signaling fragment” refers to a polypeptide comprising the transmembrane and/or intracellular region and optionally the extracellular hinge/connecting peptide regions of a cell signaling receptor. Exemplary signaling chains include the constant chains of TCRα, TCRβ, TCRγ and TCRδ. Additional exemplary signaling chains include chains comprising the transmembrane and/or intracellular regions of CD16, NKp30, NKp44, NKp46, DAP10, DAP12, DNAM-1, NKG2D, CD32, CD64, KIR3DL1, KIR2DS4, FcRγ and CD3z. In some embodiments, the signaling chain also comprise the hinge domains or the connecting peptides of CD16, NKp30, NKp44, NKp46, DAP10, DAP12, DNAM-1, NKG2D, CD32, CD64, KIR3DL1, KIR2DS4, FcRγ and CD3z.

The term “Synthetic Antigen Receptor” or “SAR” refers to a non-naturally occurring receptor or a synthetic receptor that can be expressed on the surface of a cell and comprises at least one heterologous antigen binding domain and at least one membrane associated domain, wherein the membrane associated domain can be a transmembrane domain or a membrane anchoring domain (i.e., a GPI linked domain). The antigen binding domain of the SAR is heterologous to its membrane associated domain, i.e., the antigen binding domain is derived from a different source than the membrane associated domain. A SAR may further comprise a hinge domain, an extracellular ligand binding domain and/or a cytosolic domain. In an embodiment, a SAR comprises a polypeptide or a set of polypeptides, which when expressed in an effector cell, provides the cell with specificity for a target cell, typically a cancer cell, and with intracellular signal generation. A SAR can be single chain, two chains or more than two chains. A SAR can be unispecific, bispecific or multispecific. A SAR may have one or more heterologous antigen binding domains. The term “SAR” includes conventional chimeric antigen receptors (e.g., 2^{nd} generation CARs) and next generation CARs (e.g., SIR, cTCR, AbTCR, zSIR, HIT, TFP, TAC etc.). The current disclosure describes novel SAR compositions comprising one or more regions derived from CD16A, CD16B, CD64, CD32, NKp30, NKp44, NKp46, KIR2DL1, KIR2DL2, KIR2DL3, KIR2DL5A, KIR2DL5B, KIR3DL1, KIR3DL2, KIR3DL4, KIR2DL4, KIR2DS1, KIR2DS2, KIR2DS3, KIR2DS4, KIR2DS5, KIR3DS1, NKG2D, NKG2C, NKG2A, NKG2E, NKG2F, DNAM-1, 2B4, OX40, CD28, 4-1BB, CD27, CD81, CD2, CD5, TNFR-I, TNFR-II, Fas, CD30, CD40, CRTAM, TIGIT, CD96, SLAMF6, SLAMF7, CD100, CD160, CEACAM, ILT2, KLRG1, LAIR1, CD161, Siglec3, Siglec-7, Siglec-9, CD3ƒ, DAP10, DAP12, FcRγ, TCRαβ and TCRγδ etc. and variants and fragments thereof. The disclosure also provides SARs comprising functional variants of the above genes and/or proteins include alternative spliced isoforms and homologs from other species. The exemplary regions or fragments of the above genes and proteins that can be used in the construction of the SARs of the disclosure are provided in Tables 12-18 and 25-31 of the provisional application. The exemplary extracellular domains of native receptors are provided in SEQ ID NO:10842-10877. The SAR can be also constructed with polypeptides or fragments that have 70%, 75%, 80%, 85%, 90%, 95%, 98% or 99% homology to any of the fragments provided in Tables 12-18 and 25-31 of the provisional application. The nucleic acid and amino acid sequences of exemplary additional components (e.g., vL, vH, scFv, vHH etc.) that can be used in the construction of SAR are provided in Tables 2-11. The SAR can be also constructed with polypeptides or fragments that have 70%, 75%, 80%, 85%, 90%, 95%, 98% or 99% homology to any of the fragments provided in Tables 2-18 and 25-31 of provisional application. The exemplary SARs of the disclosure are provided in Tables 32-34 of provisional application. SARs are modular in design and additional SARs can be constructed by swapping one module of the SAR with a different module. The expression and activity of these novel SARs can be tested using methods described in the disclosure to select the SARs with optimal functional activities.

The term “single-chain synthetic antigen receptor” or “single chain SAR” means a synthetic antigen receptor comprising a single polypeptide chain. An exemplary single chain SAR targeting CD19 and based on CD16 signaling chain is CD8SP-CD19-hu-mROO5-1-(vL-vH)-CD16A-F158V-S197P-FL-v3 (SEQ ID NO: 4954). In this SAR, a humanized scFv (hu-mROO5-1) targeting CD19 is operationally linked to the extracellular, transmembrane and cytosolic domains of a high affinity non-cleavable mutant of CD16 carrying F158V and S197P mutations (CD16A-F158V-S197P-FL-v3). Additional exemplary single chain SARs in which different antigen binding domains are operationally linked to the CD16A-F158V-S197P-FL-v3 module are provided in Tables 36-39 of provisional application. Additional exemplary single chain SARs are provided in SEQ ID NO: 5463-5482. As SAR are modular in design, the CD16A-F158V-S197P-FL-v3 module (SEQ ID NO (DNA): 1417 and SEQ ID NO (PRT): 3811) can be replaced by other modules, such as NKp30-ECDTMCP-opt2 (SEQ ID NO: 1375), NKp44-ECDTMCP-opt2 (SEQ ID NO: 1389), NKp46-ECDTMCP-opt2 (SEQ ID NO: 1405), CD8-hinge-NKG2D-TM-2B4CP-opt-2 (SEQ ID NO: 1434), CD32-ECDTMCP-opt2 (SEQ ID NO: 1582), CD64-ECDTMCP-opt2 (SEQ ID NO: 1584), 2B4-ECDTMCP-opt2 (SEQ ID NO: 1580), OX40-ECDTMCP-opt2 (SEQ ID NO: 1578), CD28-ECDTMCP-opt2 (SEQ ID NO:1576), 41BB-ECDTMCP-opt2 (SEQ ID NO: 1574), KIR3DL1 (SEQ ID NO: 9644) and KIR2DS4 (SEQ ID NO: 9653) etc. to generate single chain SARs with different signaling chains. Exemplary modules derived from naturally occurring receptors that can be used in the construction of SARs are provided in SEQ ID NO: 9635-9668. Exemplary such SARs are provided in SEQ ID NO: 9860-9895 and in Table 41 of the provisional application. The expression and activity of these novel SARs can be tested using methods described in the disclosure to select the SARs with optimal functional activities.

The term “Synthetic Immune Receptor” or alternatively a “SIR” refers to a set of polypeptides, typically two in some embodiments, which when expressed in an effector cell, provides the cell with specificity for a target cell, typically a cancer cell, and with intracellular signal generation. SIRs represent next generation CAR platforms that are described in WO 2018/102795 A1 which is incorporated herein by reference. In a typical embodiment, a SIR comprises one or more antigen binding domains (e.g., antibody or antibody fragment, a ligand or a receptor) that bind to antigens as described herein, and are joined to one or more T cell receptor constant chains or regions via an optional linker. In some embodiments, the set of polypeptides are contiguous with each other. In some embodiments, a SIR comprises two or more sets of two or more polypeptides. The polypeptides of each set of SIRs are contiguous with each other (functional polypeptide unit 1) but are not contiguous with the polypeptides of the other set (functional polypeptide unit 2). In some embodiments, the T cell receptor constant chains (or regions) of the SIR is chosen from the constant chain of human T cell receptor-alpha (TCR-alpha or TCRα or TCRα or hTCR-alpha or hTCRα or hTCRα or Cα), human T cell receptor-beta1(TCR-beta1 or TCRβ1 or TCRb1 or hTCR-beta1 or hTCRP1 or hTCRb1 or Cβ1), human T cell receptor-beta 2 (TCR-beta2 or TCRβ2 or TCRb2 or hTCR-beta2 or hTCRP2 or hTCRb2 or Cβ2 also designated TCR-beta, TCRβ or TCRb or Cβ), human Pre-T cell receptor alpha ((preTCR-alpha or preTCRα or preTCRα or preCα), human T cell receptor-gamma (TCR-gamma or TCRγ or TCRg or hTCR-gamma or hTCRγ or hTCRg or hTCRγ1 or hTCRgamma1, or Cγ), or human T cell receptor-delta (TCR-delta or TCRd or TCRS or hTCR-delta or hTCRd or hTCRδ or Cδ). In some embodiments, the TCR constant chains of SIR are encoded by their wild-type nucleotide sequences while in other aspects the TCR constant chains of SIR are encoded by the nucleotide sequences that are not wild-type. In some embodiments, the TCR constant chains of SIR are encoded by their codon optimized sequences. In some embodiments, the TCR constant chains of SIR encode for the wild-type polypeptide sequences while in other embodiments the TCR constant chains of SIR encoded for polypeptides that carry one or more mutations. In some embodiments, the TCR constant chains of SIR are encoded by their codon optimized sequences that carry one or more mutations. The disclosure also covers deletion mutants of TCR constant chains that retain at least one of the biological and functional properties of the corresponding full-length TCR chain. A SIR that comprises an antigen binding domain (e.g., a scFv, or vHH) that targets a specific tumor maker “X”, such as those described herein, is also referred to as X-SIR or XSIR. For example, a SIR that comprises an antigen binding domain that targets CD19 is referred to as CD19-SIR or CD19SIR. The TCR constant chain/domain of a SIR can be derived from the same species in which the SIR will ultimately be used. For example, for use in humans, it may be beneficial for the TCR constant chain of the SIR to be derived from or comprised of human TCR constant chains. However, in some instances, it is beneficial for the TCR constant chain to be derived from the same species in which the SIR will ultimately be used in, but modified to carry amino acid substitutions that enhance the expression of the TCR constant chains. For example, for use in humans, it may be beneficial for the TCR constant chain of the SIR to be derived from or comprised of human TCR constant chains but in which certain amino acids are replaced by the corresponding amino acids from the murine TCR constant chains. Such murinized TCR constant chains provide increased expression of the SIR. The SIR or functional portion thereof, can include additional amino acids at the amino or carboxy terminus, or at both termini, which additional amino acids are not found in the amino acid sequence of the TCR or antigen binding domain which make up the SIR. Desirably, the additional amino acids do not interfere with the biological function of the SIR or functional portion, e.g., recognize target cells, detect cancer, treat or prevent cancer, etc. More desirably, the additional amino acids enhance the biological activity, as compared to the biological activity of the parent SIR.

The term SVH domain as used herein refers to a single human VH domain antibody (V_{H} sdAb). These terms are thus used interchangeably. The term SVH is also used interchangeably with independent vH domains. A SVH is an example of an autonomous antigen binding domain (AABD). An exemplary SVH is a fully human vH domain (FHVH) presented in SEQ ID NO (DNA): 827-828 and SEQ ID NO (PRT): 3221-3222. Another exemplary SVH is a chVH domain presented in SEQ ID NO (DNA): 830-831 and SEQ ID NO (PRT): 8223-8224. Another exemplary SVH is an aVH domain presented in SEQ ID NO (DNA): 850-851 and SEQ ID NO (PRT): 3244-3245. The SEQ ID numbers of other exemplary SVH domains are presented in Table 5. Additional SVH domains that can be used in the construction of the SARs of the disclosure are provided in WO2016062988, WO2016113556, WO2017191476, WO2018039180, WO2019006072, WO2018237037, WO2018119215, WO2019126756, WO2019055689 and WO2020018922, which are incorporated in their entirety by reference herein.

The term “stimulation,” refers to a primary response induced by binding of a stimulatory molecule (e.g., a TCR/CD3 complex) with its cognate ligand (or target antigen) thereby mediating a signal transduction event, such as, but not limited to, signal transduction via the TCR/CD3. Stimulation can mediate altered expression of certain molecules.

The term “stimulatory molecule,” refers to a molecule expressed by an immune cell (e.g., T cell, NK cell, B cell) that provides the cytoplasmic signaling sequence(s) that regulate activation of the immune cell in a stimulatory way for at least some aspect of the immune cell signaling pathway. In one aspect, the signal is a primary signal that is initiated by, for instance, binding of a TCR/CD3 complex with an MHC molecule loaded with peptide, and which leads to mediation of a T cell response, including, but not limited to, proliferation, activation, differentiation, and the like. A primary cytoplasmic signaling sequence (also referred to as a “primary signaling domain”) that acts in a stimulatory manner may contain a signaling motif which is known as immunoreceptor tyrosine-based activation motif or ITAM. Examples of an ITAM containing cytoplasmic signaling sequence includes, but is not limited to, those derived from CD3 zeta, FcRy, CD3 gamma, CD3 delta, CD3 epsilon, CD79a, CD79b, DAP10, and DAP12.

The term “subject” is intended to include living organisms in which an immune response can be elicited (e.g., any domesticated mammals or a human). The terms “subject” or “individual” or “animal” or “patient” are used interchangeably herein to refer to any subject, particularly a mammalian subject, for whom administration of a composition or pharmaceutical composition of the disclosure is desired. Mammalian subjects include humans, non-human primates, dogs, cats, guinea pigs, rabbits, rats, mice, horses, cattle, cows, and the like, with humans being preferred.

“Switch domain,” or a “dimerization domain” as used herein, typically refers to a polypeptide-based entity that, in the presence of a dimerization molecule, associates with another switch domain.

A “suicide gene”, “suicide switch” or a “Kill-switch” encodes for a protein which possesses an inducible capacity to lead to cellular death. Exemplary suicide genes include HSV-TK, iCaspase 9, tEGFR, CD20, tCD19, tHer2, tBCMA, RQR8 etc. For example, CD20-expressing cells can be selectively ablated by treatment with the antibody Rituximab. Similarly, tBCMA expressing cells can be selectively ablated by treatment with belantamab mafodotin and tHer2 expressing cells can be selectively ablated by treatment with Herceptin.

A “survival gene”, “survival switch” or a “Life-switch” encodes for a protein that provides a pro-survival signal to a cell. Exemplary survival genes include membrane anchored form of IL2 and membrane anchored form of IL15.

As used herein, the term “T lymphocyte” or “T cell” refers to a cell expressing CD3 (CD3+) and a T Cell Receptor (TCR+). In an embodiment, a T cell is a native cell (i.e., a cell that is not a recombinant or engineered) that expresses CD3 and a TCR.

As used herein, the term “TCR” or “T cell receptor” refers to a dimeric heterologous cell surface signaling protein forming an alpha-beta or gamma-delta receptor typically involved in recognizing an antigen presented by an MHC molecule (i.e., antigen recognition in the context of an MHC molecule). TCRs of the disclosure may be non-naturally occurring and/or purified and/or engineered. TCRs of the disclosure may have more than one mutation present in the alpha chain variable domain and/or the beta chain variable domain relative to the parental TCR. “Engineered TCR” and “mutant TCR” are used synonymously herein and generally mean a TCR which has one or more mutations introduced relative to the parental TCR, in particular in the Va and/or Vb or Vg and/or Vd domain thereof. An engineered TCR may bind to an antigen in an HLA-dependent or HLA-independent manner.

As used herein, the term “transgene” refers to a heterologous gene that is integrated into the genome of an organism (e.g., a non-human animal) and that is transmitted to progeny of the organism during sexual reproduction.

As used herein, the term “T lymphocyte” or “T cell” refers to a cell expressing CD3 (CD3+) and a T Cell Receptor (TCR+). In an embodiment, a T cell is a native cell (i.e., a cell that is not engineered to express CD3 or TCR) that expresses CD3 and a TCR naturally. The terms “T cell” and “T lymphocyte” are interchangeable and used synonymously herein. Examples include but are not limited to naïve T cells (“lymphocyte progenitors”), central memory T cells, effector memory T cells, stem memory T cells (T_{scm}), iPSC-derived T cells, synthetic T cells, tumor infiltrating T cells (TIL), αβ T cells, γδ T cells, regulatory T cells (Tregs) or combinations thereof.

The term “non-T cell” refers to a cell that is not a T cell. In an embodiment, a non-T cell lacks the cell surface expression of CD3 and a T cell receptor. In an embodiment, a non-T cell does not respond to a T cell activating antibody, such as OKT3. In an embodiment, a non-T cell lacks surface expression of CD3. In an embodiment, a non-T cell lacks the expression of one or more of CD3 chains selected from the group of CD3ε, CD3γ and CD3δ. In an embodiment, a non-T cell shows germline configuration of TCR genes and has not undergone T cell gene rearrangement. In an embodiment, a non-T cell lacks the ability to form a functional T cell/CD3 receptor complex. An exemplary non-T cell includes an NK cell, a B cell, a macrophage, a granulocyte, a dendritic cell and an epithelial cell. A non-T cell can be an immortalized cell line. In an exemplary embodiment, a non-T cell is an NK cell lines, e.g., NK92, NK92MI, NKG and YTS etc. In an embodiment, a non-T cell is an iPSC derived cell that lacks CD3 and T cell receptor expression.

The term “T cell receptor module,” or “TCRM,” refers to a heterodimer comprising sequences derived from a T cell receptor. The TCRM comprises T cell receptor transmembrane domains and may further comprise all or a portion of T cell receptor connecting peptides and/or intracellular domains.

The term “TCR-Fv” or “Fv-TCR” of “fragment variable TCR” as used here refers to an antigen binding module that is formed by the variable domains of TCR chains. A TCR-Fv can be formed by the Vα and Vβ domains or by Vγ and Vδ domains. A TCR-Fv shows some or all the specific binding affinity for a target antigen (e.g., peptide/MHC complex) of the TCR from which the variable domains are derived. In an embodiment, the SAR of the disclosure demonstrate the ability to form a TCR-Fv antigen binding module when the Vα/Vβ or Vγ/Vδ chains derived from a TCR are attached to its two polypeptides.

The term “Fv” or “fragment variable” as used here refers to an antigen binding module that is formed by the variable domains of an antibody. A Fv can be formed by the vL and vH domains. A Fv shows some or all the specific binding affinity for a target antigen of the antibody from which the variable domains are derived. In an embodiment, the SAR of the disclosure demonstrate the ability to form a Fv antigen binding module when the vL and vH chains derived from an antibody are attached to its two polypeptides.

As used herein, a “transmembrane protein” or “membrane protein” is a protein located at and/or within a membrane such as the phospholipid bilayer of a biological membrane (e.g., biomembranes such as the membrane of a cell). Some proteins are bound only to the membrane surface, whereas others have one or more regions buried within the membrane and/or domains on one or both sides of the membrane. Specific examples of transmembrane proteins include CD8a, CD4, CD3ƒ, CD16, NKp30, NKp44, NKG2D etc.

As used herein a “transmembrane module” or “TMM” refers to a molecule or a molecular complex comprising a transmembrane protein (e.g., CD16A).

The term “membrane associated module” or “MAM” refers to a molecule or a molecular complex comprising a transmembrane protein (e.g., CD16A) or a membrane anchored protein (e.g., CD16B). The term encompasses transmembrane proteins, such as CD16A, and GPI (glycosylphosphatidylinositol) linked proteins, such as CD16B. A MAM may further comprise all or portions of hinge domains and/or cytosolic domains.

“Therapeutic agents” as used herein refers to agents that are used to, for example, treat, inhibit, prevent, mitigate the effects of, reduce the severity of, reduce the likelihood of developing, slow the progression of and/or cure, a disease. Diseases targeted by therapeutic agents include but are not limited to infectious diseases, Carcinomas, sarcomas, lymphomas, leukemia, germ cell tumors, blastomas, antigens expressed on various immune cells, and antigens expressed on cells associated with various hematologic diseases, and/or inflammatory diseases.

“Therapeutic Controls” as used herein refers to an element used for controlling the activity of a SAR expressing cell. In some embodiments, therapeutic controls for controlling the activity of the SAR expressing cells of the disclosure comprise any one or more of truncated epidermal growth factor receptor (tEGFR), truncated epidermal growth factor receptor viii (tEGFRviii), truncated CD30 (tCD30), truncated BCMA (tBCMA), truncated CD19 (tCD19), thymidine kinase, cytosine deaminase, nitroreductase, xanthine-guanine phosphoribosyl transferase, human caspase 8, human caspase 9, inducible caspase 9, purine nucleoside phosphorylase, linamarase/linamarin/glucose oxidase, deoxyribonucleoside kinase, horseradish peroxidase (HRP)/indole-3-acetic (IAA), Gamma-glutamylcysteine synthetase, CD20/alphaCD20, CD34/thymidine kinase chimera, dox-dependent caspase-2, mutant thymidine kinase (HSV-TKSR39), AP1903/Fas system, a chimeric cytokine receptor (CCR), a selection marker, and combinations thereof. Exemplary therapeutic controls are provided in Table 24 of provisional application.

The term “therapeutic effect” refers to a biological effect which can be manifested by various means, including but not limited to, e.g., decrease in tumor volume, a decrease in the number of cancer cells, a decrease in the number of metastases, an increase in life expectancy, decrease in cancer cell proliferation, decrease in cancer cell survival, decrease in the titer of the infectious agent, a decrease in colony counts of the infectious agent, amelioration of various physiological symptoms associated with a disease condition. A “therapeutic effect” can also be manifested by the ability of the peptides, polynucleotides, cells and antibodies in prevention of the occurrence of disease in the first place or in the prevention of relapse of the disease.

The term “therapeutically effective amount” as used herein refers to the amount of a pharmaceutical composition comprising one or more peptides as disclosed herein or a mutant, variant, analog or derivative thereof, to decrease at least one or more symptom of the disease or disorder, and relates to a sufficient amount of pharmacological composition to provide the desired effect. The phrase “therapeutically effective amount” as used herein means a sufficient amount of the composition to treat a disorder, at a reasonable benefit/risk ratio applicable to any medical treatment.

The term “TCR receptor fusion proteins” or “TFP” refers to a next generation SAR platform as described in WO 2016/187349 A1 which is incorporated herein by reference. In an embodiment, a TFP comprises an antibody moiety that specifically binds to a target antigen fused to a TCR chain such as CD3ε, CD3γ, CD36, TCRα or TCRβ. Exemplary TCR chains that can be used in the construction of TFP are represented by SEQ ID NOs: 11903-11906 of this disclosure and are provided in WO 2017/070608 A1 which is incorporated herein by reference. A TFP incorporating CD3ε chain is referred to as a CD3E TFP or TFPε. A TFP incorporating CD3γ chain is referred to as a CD3γ TFP or TFPγ. A TFP incorporating CD3δ chain is referred to as a CD3δ TFP or TFPδ. The TFP incorporating CD3ε, CD3γ or CD3δ chains are collectively referred to as CD3ε/y/δ TFP or TFPε/γ/δ.

The term “transfer vector” refers to a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “transfer vector” includes an autonomously replicating plasmid or a virus. The term should also be construed to further include non-plasmid and non-viral compounds which facilitate transfer of nucleic acid into cells, such as, for example, a poly lysine compound, liposome, and the like. Examples of viral transfer vectors include, but are not limited to, adenoviral vectors, adeno-associated virus vectors, retroviral vectors, lentiviral vectors, and the like.

“Transmembrane domain” (TMD) as used herein refers to the region of a receptor, (e.g., a SAR) which crosses the plasma membrane. The transmembrane domain of the SAR of the disclosure is the transmembrane region of a transmembrane protein (for example Type I transmembrane protein or Type II transmembrane protein), an artificial hydrophobic sequence or a combination thereof. Other transmembrane domains will be apparent to those of skill in the art and may be used in connection with alternate embodiments of the disclosure. In some embodiments, the TMD encoded SAR comprises a transmembrane domain selected from the transmembrane domain of an alpha, beta or zeta chain of a T-cell receptor, CD3γ, CD3ε, CD36, CD28, CD45, CD4, CD5, CD8, CD9, CD16, CD22, CD33, CD37, CD64, CD80, CD86, CD134, CD137, CD154, KIRDS2, OX40, CD2, CD27, LFA-1 (CDl la, CD18), ICOS (CD278), 4-1BB (CD137), GITR, CD40, BAFFR, HVEM (LIGHTR), SLAMF7, NKp80 (KLRFl), CD160, CD19, IL2R beta, IL2R gamma, IL7R a, ITGA1, VLA1, CD49a, ITGA4, IA4, CD49D, ITGA6, VLA-6, CD49f, ITGAD, CDl ld, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1(CD226), SLAMF4 (CD244, 2B4), CD84, CD96 (Tactile), CEACAMI, CRT AM, Ly9 (CD229), CD160 (BY55), PSGL1, CD100 (SEMA4D), SLAMF6 (NTB-A, Lyl08), SLAM (SLAMFI, CD150, IPO-3), BLAME (SLAMF8), SELPLG (CD162), LTBR, PAG/Cbp, NKp44, NKp30, NKp46, NKG2D, and/or NKG2C. Exemplary transmembrane domains are provided in Table 28 of provisional application. The transmembrane domain of the SAR of the disclosure may be native or non-native to the receptor. As the SARs are modular in design, in some embodiments the transmembrane domain of one SAR may be replaced by transmembrane domain of another SAR as long as it retains its biological and functional properties. Thus, the NKp30 transmembrane domain in a NKp30-based SAR may be replaced by the transmembrane domain of NKp44. The resulting SAR with a non-native transmembrane domain can be tested for its cell surface expression and functional activities using assays known in the art and assays described in this disclosure.

As used herein “Tri-functional T cell antigen coupler” or “Tri-TAC” or “TAC” refer to a next generation SAR platform described in WO 2015/117229 A1, which is incorporated herein by reference. Tri-TAC targeting different antigens can be constructed using the antigen binding domains (e.g., vL and vH fragments, scFv, vHH, ligands and receptors etc.) described in this disclosure using techniques known in the art.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with, a disease or disorder.

“Tumor,” as used herein refers to all neoplastic cell growth and proliferation, whether malignant or benign, and all pre-cancerous and cancerous cells and tissues.

“Vector”, “cloning vector” and “expression vector” as used herein refer to the vehicle by which a polynucleotide sequence (e.g., a foreign gene) can be introduced into a host cell, so as to transform the host and promote expression (e.g., transcription and translation) of the introduced sequence. Vectors include plasmids, phages, viruses, etc.

The term “viral vector” refers to a vector obtained or derived from a virus. Typically the virus is a retrovirus including, but not limited to, lentiviruses and gamma retroviruses. The viral vector of the disclosure may be a retroviral vector, such as a gamma-retroviral vector. The viral vector may be based on human immunodeficiency virus. The viral vector of the disclosure may be a lentiviral vector. The vector may be based on a non-primate lentivirus such as equine infectious anemia virus (EIAV). The viral vector of the disclosure comprises a mitogenic T-cell activating transmembrane protein and/or a cytokine-based T-cell activating transmembrane protein in the viral envelope. The mitogenic T-cell activating transmembrane protein and/or cytokine-based T-cell activating transmembrane protein is/are derived from the host cell membrane, as explained above.

The term “zeta” or alternatively “zeta chain”, “CD3-zeta” or “TCR-zeta” “CD3ƒ “is defined as the protein provided as GenBank Ace. No. BAG36664.1, or the equivalent residues from a non-human species, e.g., mouse, rodent, monkey, ape and the like, and a “zeta stimulatory domain” or alternatively a “CD3-zeta stimulatory domain” or a “TCR-zeta stimulatory domain” is defined as the amino acid residues from the cytoplasmic domain of the zeta chain, or functional derivatives thereof, that are sufficient to functionally transmit an initial signal necessary for T cell activation.

“HLA deficient”, including HLA-class I deficient, or HLA-class II deficient, or both, refers to cells that either lack, or no longer maintain, or have reduced level of surface expression of a complete MHC complex comprising an HLA class I protein heterodimer and/or an HLA class II heterodimer, such that the diminished or reduced level is less than the level naturally detectable by other cells or by synthetic methods.

“Modified HLA deficient iPSC,” as used herein, refers to HLA deficient iPSC that is further modified by introducing genes expressing proteins related but not limited to improved differentiation potential, antigen targeting, antigen presentation, antibody recognition, persistence, immune evasion, resistance to suppression, proliferation, costimulation, cytokine stimulation, cytokine production (autocrine or paracrine), chemotaxis, and cellular cytotoxicity, such as non-classical HLA class I proteins (e.g., HLA-E and HLA-G), chimeric antigen receptor (CAR), T cell receptor (TCR), CD16 Fc Receptor, BCL1 lb, NOTCH, RUNX1, IL15, 41BB, DAP 10, DAP 12, CD24, CD3z, 41BBL, CD47, CD 113, and PDL1. The cells that are “modified HLA deficient” also include cells other than iPSCs.

CD16, a FcγR receptor, has been identified to have two isoforms, Fc receptors FcγRIIIa (CD16a) and FcγRIIIb (CD16b). Unless specified otherwise, CD16 refers to both CD16a and CD16b isoforms and any other alternatively spliced variant from human or non-human species. CD16a is a transmembrane protein expressed by NK cells, which binds monomeric IgG attached to target cells to activate NK cells and facilitate antibody-dependent cell-mediated cytotoxicity (ADCC). “High affinity CD16, ““non-cleavable CD16,” or “high affinity non-cleavable CD16 (hnCD16),” as used herein, refers to a natural or non-natural variant of CD 16. The wildtype CD16 has low affinity and is subject to extodomain shedding, a proteolytic cleavage process that regulates the cells surface density of various cell surface molecules on leukocytes upon NK cell activation. F176V (or F158V or V158) are exemplary CD16 polymorphic variants having high affinity. A CD16 variant having the cleavage site (position 195-198) in the membrane-proximal region (position 189-212) altered or eliminated is not subject to shedding. The cleavage site and the membrane-proximal region are described in detail in WO2015148926, the complete disclosures of which are incorporated herein by reference. The CD16 S197P or S197R variant is an engineered non-cleavable version of CD16. A CD16 variant comprising both F158V and S197P (or S197R) has high affinity and is non-cleavable. Another exemplary high affinity and non-cleavable CD16 (hnCD16) variant is an engineered CD 16 comprising an ectodomain originated from one or more of the 3 exons of the CD64 ectodomain. The CD16 SAR of the disclosure may comprise the wildtype CD16 sequence or its natural or non-natural variants, such a F158V and S197P (or S197R),

While immune cells and iPSC expressing CD16 or CD16 variants are known in the art, in one embodiment, the disclosure provides SARs comprising the extracellular Fc binding region of CD16 or CD16 variants. In an embodiment, a CD16-SAR retains the ability to bind Fc region of an antibody or antibody fragment but has the additional ability to bind to an antigen through its non-natural antigen binding domain (e.g., AABD, scFv, vHH, FHVH, Fv etc.). In an embodiment, the antigen binding domain of the CD16 SAR is operably linked to the N-terminal region or near the N-terminal region of the D1 domain (SEQ ID NO: 3836) of CD16 or the CD16 variants, i.e., at or near the N-terminal region of the extracellular domain of CD16 or CD16 variants. In an embodiment, an optional linker is present between the antigen binding domain of the SAR and the D1 domain of CD16 or CD16 variant. Exemplary linkers are provided in Table 11 of provisional application.

In an embodiment, a CD16-SAR lacks the ability to bind Fc region of an antibody or antibody fragment but possess the ability to bind to an antigen through its non-natural antigen binding domain (e.g., AABD, scFv, vHH, FHVH, Fv etc.). In an exemplary embodiment, a CD16-SAR comprises one or more non-natural antigen binding domains (e.g., AABD, scFv, vHH, FHVH, Fv etc.) that are operationally linked via an optional hinge domain to the transmembrane and optionally the cytosolic domain of CD16. In an embodiment, a CD16-SAR possesses the ability to recruit signaling adaptors, such as CD3z and/or FceRy1 upon binding to its target antigen. The binding domain of a SAR binds to a desired epitope or antigen. For example, the epitope recognized by a SAR is determined from the epitope recognized by the scFv used as the binding domain of the SAR. For example, since the antigen specific domain of the SAR CD8SP-CD19-hu-mROO5-1-vL-Xho-IgCL-DAP10-opt1-F-P2A-Spe-IgSP-Bst-CD19-hu-mROO5-1-vH-Mlu-IglCH1-DAP10-opt2-F-F2A-Xba-PAC (SEQ ID NO: 2275) targeting CD19 is comprised of vL (SEQ ID NO: 2543) and vH (SEQ ID NO: 2785) fragments derived from hu-mROO5-1 scFv (SEQ ID NO: 3027), it is expected that the SAR targets the same epitope as the scFv and/or the parental antibody from which the scFv is derived. The epitopes recognized by several scFv and/or their parental antibodies used in the construction of the SARs and backbones of this disclosure are known in the art. Alternatively, the epitope targeted by a SAR can be determined by generating a series of mutants of its target antigen and testing the ability of the mutants to bind to the SAR-expressing cells using techniques known in the art, for example, using the Topanga Assay (Gopalakrishnan, R, Sci Reports, 2019). As an example, the epitope recognized by the SAR CD8SP-CD19-hu-mROO5-1-vL-Xho-IgCL-DAP10-opt1-F-P2A-Spe-IgSP-Bst-CD19-hu-mROO5-1-vH-Mlu-Ig1CH1-DAP10-opt2-F-F2A-Xba-PAC (SEQ ID NO: 2275) targeting CD19 can be determined by generating a panel of deletion and point mutants of the CD19-ECD-GGSG-NLuc-4xFlag-2xStreptag-8xHis-T2A-Pac (DNA SEQ ID NO: 1282). The mutant constructs would be transfected into a suitable cell line (e.g., 293FT cells) and the supernatant containing the fusion protein collected and assayed for NLuc activity to assure that the different mutant CD19-ECD-GGSG-NLuc-4xFlag-2xStreptag-8xHis fusion proteins are being secreted in the supernatant. Subsequently, the fusion proteins would be tested for their ability to bind to cells (e.g., Jurkat cells or T cells) expressing the CD8SP-CD19-hu-mROO5-1-vL-Xho-IgCL-DAP10-opt1-F-P2A-Spe-IgSP-Bst-CD19-hu-mROO5-1-vH-Mlu-Ig1CH1-DAP10-opt2-F-F2A-Xba-PAC (SEQ ID NO: 2275) construct. The mutant that fails to bind to the SAR-expressing cells is a candidate for containing the epitope targeted by the CD19-specific SAR. An alternate approach to determine the epitope recognized by a particular SAR could include a functional competitive assay with different test antibodies. For example, T cells expressing the CD8SP-CD19-hu-mROO5-1-vL-Xho-IgCL-DAP10-opt1-F-P2A-Spe-IgSP-Bst-CD19-hu-mROO5-1-vH-Mlu-Ig1CHI-DAP10-opt2-F-F2A-Xba-PAC (SEQ ID NO: 2275) SAR could be co-cultured with a cell line expressing CD19 (e.g., RAJI cells) in the absence and presence of increasing concentrations of different test CD19 antibodies. In case the epitope recognized by a test CD19 antibody overlaps with the epitope recognized by the SAR CD8SP-CD19-hu-mROO5-1-vL-Xho-IgCL-DAP10-opt1-F-P2A-Spe-IgSP-Bst-CD19-hu-mROO5-1-vH-Mlu-Ig1CHI-DAP10-opt2-F-F2A-Xba-PAC (SEQ ID NO: 2275), then the test antibody would be expected to block target-cell killing and cytokine production induced by T cells expressing the CD8SP-CD19-hu-mROO5-1-vL-Xho-IgCL-DAP10-opt1-F-P2A-Spe-IgSP-Bst-CD19-hu-mROO5-1-vH-Mlu-Ig1CH1-DAP10-opt2-F-F2A-Xba-PAC (SEQ ID NO: 2275) SAR in a dose-dependent manner. A non-specific antibody of the same isotype as the test antibody would be included as a control and would be expected to have no effect on the target-cell killing and cytokine production induced by T cells expressing the SAR. Similarly, a specific SAR can be expressed in Jurkat-NFAT-EGFP cells and the ability of a test antibody to block EGFP induction by the SAR-expressing Jurkat-NFAT-GFP cells upon coculture with a target cell line can be used to determine whether the epitope recognized by the test antibody overlaps with the epitope recognized by the said SAR.

TABLE 3

| | | | | | scFV | |
|---|---|---|---|---|---|---|
| | vL | | vH | | scFv- | |
| | vL SEQ | vL SEQ | vH SEQ | vH-SEQ | DNA | scFv- |
| TARGET | ID NO | ID NO | ID NO | ID NO | SEQ ID | PRT |
| ANTIGEN | (DNA) | (PRT) | (DNA) | (PRT) | NO | SEQ ID |
| ALK | 46 | 2440 | 288 | 2682 | 530 | 2924 |
| ALK | 47 | 2441 | 289 | 2683 | 531 | 2925 |
| BCMA | 48 | 2442 | 290 | 2684 | 532 | 2926 |
| BCMA | 49 | 2443 | 291 | 2685 | 533 | 2927 |
| CD123 | 50 | 2444 | 292 | 2686 | 534 | 2928 |
| CD138 | 51 | 2445 | 293 | 2687 | 535 | 2929 |
| CD179b | 52 | 2446 | 294 | 2688 | 536 | 2930 |
| CD19 | 53 | 2447 | 295 | 2689 | 537 | 2931 |
| CD19 | 54 | 2448 | 296 | 2690 | 538 | 2932 |
| CD19 | 55 | 2449 | 297 | 2691 | 539 | 2933 |
| CD20 | 56 | 2450 | 298 | 2692 | 540 | 2934 |
| CD20 | 57 | 2451 | 299 | 2693 | 541 | 2935 |
| CD22 | 58 | 2452 | 300 | 2694 | 542 | 2936 |
| CD276 | 59 | 2453 | 301 | 2695 | 543 | 2937 |
| CD30 | 60 | 2454 | 302 | 2696 | 544 | 2938 |
| CD30 | 61 | 2455 | 303 | 2697 | 545 | 2939 |
| CD32 | 62 | 2456 | 304 | 2698 | 546 | 2940 |
| CD324 | 63 | 2457 | 305 | 2699 | 547 | 2941 |
| CD324 | 64 | 2458 | 306 | 2700 | 548 | 2942 |
| CD33b | 65 | 2459 | 307 | 2701 | 549 | 2943 |
| CD33 | 66 | 2460 | 308 | 2702 | 550 | 2944 |
| CD34 | 67 | 2461 | 309 | 2703 | 551 | 2945 |
| CD5 | 68 | 2462 | 310 | 2704 | 552 | 2946 |
| CD5 | 69 | 2463 | 311 | 2705 | 553 | 2947 |
| CD70 | 70 | 2464 | 312 | 2706 | 554 | 2948 |
| CD79b | 71 | 2465 | 313 | 2707 | 555 | 2949 |
| CD79b | 72 | 2466 | 314 | 2708 | 556 | 2950 |
| CDH17 | 73 | 2467 | 315 | 2709 | 557 | 2951 |
| CDH19 | 74 | 2468 | 316 | 2710 | 558 | 2952 |
| CDH6 | 75 | 2469 | 317 | 2711 | 559 | 2953 |
| CDH6 | 76 | 2470 | 318 | 2712 | 560 | 2954 |
| CLEC5A | 77 | 2471 | 319 | 2713 | 561 | 2955 |
| CLEC5A | 78 | 2472 | 320 | 2714 | 562 | 2956 |
| CLL1 | 79 | 2473 | 321 | 2715 | 563 | 2957 |
| CLL1 | 80 | 2474 | 322 | 2716 | 564 | 2958 |
| CS1/SLAMF7 | 81 | 2475 | 323 | 2717 | 565 | 2959 |
| CS1/SLAMF7 | 82 | 2476 | 324 | 2718 | 566 | 2960 |
| CSF2RA | 83 | 2477 | 325 | 2719 | 567 | 2961 |
| CSF2RA | 84 | 2478 | 326 | 2720 | 568 | 2962 |
| DLL3 | 85 | 2479 | 327 | 2721 | 569 | 2963 |
| DLL3 | 86 | 2480 | 328 | 2722 | 570 | 2964 |
| EGFR | 87 | 2481 | 329 | 2723 | 571 | 2965 |
| EGFRviii | 88 | 2482 | 330 | 2724 | 572 | 2966 |
| EpCAM | 89 | 2483 | 331 | 2725 | 573 | 2967 |
| EpCAM | 90 | 2484 | 332 | 2726 | 574 | 2968 |
| FLT3 | 91 | 2485 | 333 | 2727 | 575 | 2969 |
| HIV1-gp | 92 | 2486 | 334 | 2728 | 576 | 2970 |
| FR-1 | 93 | 2487 | 335 | 2729 | 577 | 2971 |
| GD2 | 94 | 2488 | 336 | 2730 | 578 | 2972 |
| GD2 | 95 | 2489 | 337 | 2731 | 579 | 2973 |
| GD3 | 96 | 2490 | 338 | 2732 | 580 | 2974 |
| GFR4 | 97 | 2491 | 339 | 2733 | 581 | 2975 |
| GM1 | 98 | 2492 | 340 | 2734 | 582 | 2976 |
| GPRC5D | 99 | 2493 | 341 | 2735 | 583 | 2977 |
| GPRC5D | 100 | 2494 | 342 | 2736 | 584 | 2978 |
| Her2 | 101 | 2495 | 343 | 2737 | 585 | 2979 |
| HIV1-gp100 | 102 | 2496 | 344 | 2738 | 586 | 2980 |
| HIV1-gp100 | 103 | 2497 | 345 | 2739 | 587 | 2981 |
| IL11Ra | 104 | 2498 | 346 | 2740 | 588 | 2982 |
| IL13Ra2 | 105 | 2499 | 347 | 2741 | 589 | 2983 |
| IL13Ra2 | 106 | 2500 | 348 | 2742 | 590 | 2984 |
| L1CAM | 107 | 2501 | 349 | 2743 | 591 | 2985 |
| LAMP1 | 108 | 2502 | 350 | 2744 | 592 | 2986 |
| LAMP1 | 109 | 2503 | 351 | 2745 | 593 | 2987 |
| Lym1 | 110 | 2504 | 352 | 2746 | 594 | 2988 |
| Lym2 | 111 | 2505 | 353 | 2747 | 595 | 2989 |
| MPL/TPO-R | 112 | 2506 | 354 | 2748 | 596 | 2990 |
| MPL/TPO-R | 113 | 2507 | 355 | 2749 | 597 | 2991 |
| MPL/TPO-R | 114 | 2508 | 356 | 2750 | 598 | 2992 |
| MPL/TPO-R | 115 | 2509 | 357 | 2751 | 599 | 2993 |
| TCRB1 | 116 | 2510 | 358 | 2752 | 600 | 2994 |
| TCRB1 | 117 | 2511 | 359 | 2753 | 601 | 2995 |
| TCRB2 | 118 | 2512 | 360 | 2754 | 602 | 2996 |
| TCRB2 | 119 | 2513 | 361 | 2755 | 603 | 2997 |
| TCRgd | 120 | 2514 | 362 | 2756 | 604 | 2998 |
| TnAg | 121 | 2515 | 363 | 2757 | 605 | 2999 |
| Tn-Muc1 | 122 | 2516 | 364 | 2758 | 606 | 3000 |
| TROP2 | 123 | 2517 | 365 | 2759 | 607 | 3001 |
| WT1/HLA-A2 | 124 | 2518 | 366 | 2760 | 608 | 3002 |
| WT1/HLA-A2 | 125 | 2519 | 367 | 2761 | 609 | 3003 |
| WT1/HLA-A2 | 126 | 2520 | 368 | 2762 | 610 | 3004 |
| CD123 | 127 | 2521 | 369 | 2763 | 611 | 3005 |
| CDH19 | 128 | 2522 | 370 | 2764 | 612 | 3006 |
| FRbeta | 129 | 2523 | 371 | 2765 | 613 | 3007 |
| B7J4 | 130 | 2524 | 372 | 2766 | 614 | 3008 |
| B7H4 | 131 | 2525 | 373 | 2767 | 615 | 3009 |
| CD23 | 132 | 2526 | 374 | 2768 | 616 | 3010 |
| GCC | 133 | 2527 | 375 | 2769 | 617 | 3011 |
| CD200R | 134 | 2528 | 376 | 2770 | 618 | 3012 |
| AFP/HLA-A2 | 135 | 2529 | 377 | 2771 | 619 | 3013 |
| AFP/HLA-A2 | 136 | 2530 | 378 | 2772 | 620 | 3014 |
| BCMA | 137 | 2531 | 379 | 2773 | 621 | 3015 |
| BCMA | 138 | 2532 | 380 | 2774 | 622 | 3016 |
| BCMA | 139 | 2533 | 381 | 2775 | 623 | 3017 |
| CD123 | 140 | 2534 | 382 | 2776 | 624 | 3018 |
| CD123 | 141 | 2535 | 383 | 2777 | 625 | 3019 |
| CD123 | 142 | 2536 | 384 | 2778 | 626 | 3020 |
| CD123 | 143 | 2537 | 385 | 2779 | 627 | 3021 |
| CD123 | 144 | 2538 | 386 | 2780 | 628 | 3022 |
| CD123 | 145 | 2539 | 387 | 2781 | 629 | 3023 |
| CD19 | 146 | 2540 | 388 | 2782 | 630 | 3024 |
| CD19 | 147 | 2541 | 389 | 2783 | 631 | 3025 |
| CD19 | 148 | 2542 | 390 | 2784 | 632 | 3026 |
| CD19 | 149 | 2543 | 391 | 2785 | 633 | 3027 |
| CD20 | 150 | 2544 | 392 | 2786 | 634 | 3028 |
| CD20 | 151 | 2545 | 393 | 2787 | 635 | 3029 |
| CD33 | 152 | 2546 | 394 | 2788 | 636 | 3030 |
| CD99 | 153 | 2547 | 395 | 2789 | 637 | 3031 |
| CLL1 | 154 | 2548 | 396 | 2790 | 638 | 3032 |
| CLL1 | 155 | 2549 | 397 | 2791 | 639 | 3033 |
| CLL1 | 156 | 2550 | 398 | 2792 | 640 | 3034 |
| CLL1 | 157 | 2551 | 399 | 2793 | 641 | 3035 |
| FITC | 158 | 2552 | 400 | 2794 | 642 | 3036 |
| FITC | 159 | 2553 | 401 | 2795 | 643 | 3037 |
| GPRC5D | 160 | 2554 | 402 | 2796 | 644 | 3038 |
| GPRC5D | 161 | 2555 | 403 | 2797 | 645 | 3039 |
| HLA-A2 | 162 | 2556 | 404 | 2798 | 646 | 3040 |
| Kappa-LC | 163 | 2557 | 405 | 2799 | 647 | 3041 |
| CD19 | 164 | 2558 | 406 | 2800 | 648 | 3042 |
| Streptag | 165 | 2559 | 407 | 2801 | 649 | 3043 |
| BCMA | 166 | 2560 | 408 | 2802 | 650 | 3044 |
| BCMA | 167 | 2561 | 409 | 2803 | 651 | 3045 |
| MPL/TPO-R | 168 | 2562 | 410 | 2804 | 652 | 3046 |
| CD22 | 169 | 2563 | 411 | 2805 | 653 | 3047 |
| CD22 | 170 | 2564 | 412 | 2806 | 654 | 3048 |
| CD22 | 171 | 2565 | 413 | 2807 | 655 | 3049 |
| CD22 | 172 | 2566 | 414 | 2808 | 656 | 3050 |
| CD22 | 173 | 2567 | 415 | 2809 | 657 | 3051 |
| MPL/TPO-R | 174 | 2568 | 416 | 2810 | 658 | 3052 |
| MPL/TPO-R | 175 | 2569 | 417 | 2811 | 659 | 3053 |
| CD179a | 176 | 2570 | 418 | 2812 | 660 | 3054 |
| CD179a | 177 | 2571 | 419 | 2813 | 661 | 3055 |
| CD22 | 178 | 2572 | 420 | 2814 | 662 | 3056 |
| STEAP1 | 179 | 2573 | 421 | 2815 | 663 | 3057 |
| Liv1 | 180 | 2574 | 422 | 2816 | 664 | 3058 |
| Nectin4 | 181 | 2575 | 423 | 2817 | 665 | 3059 |
| CRIPTO | 182 | 2576 | 424 | 2818 | 666 | 3060 |
| gpA33 | 183 | 2577 | 425 | 2819 | 667 | 3061 |
| ROR1 | 184 | 2578 | 426 | 2820 | 668 | 3062 |
| BCMA | 185 | 2579 | 427 | 2821 | 669 | 3063 |
| CLL1 | 186 | 2580 | 428 | 2822 | 670 | 3064 |
| CLL1 | 187 | 2581 | 429 | 2823 | 671 | 3065 |
| FLT3 | 188 | 2582 | 430 | 2824 | 672 | 3066 |
| FLT3 | 189 | 2583 | 431 | 2825 | 673 | 3067 |
| IL1RAP | 190 | 2584 | 432 | 2826 | 674 | 3068 |
| IL1RAP | 191 | 2585 | 433 | 2827 | 675 | 3069 |
| IL1RAP | 192 | 2586 | 434 | 2828 | 676 | 3070 |
| MSLN | 193 | 2587 | 435 | 2829 | 677 | 3071 |
| MSLN | 194 | 2588 | 436 | 2830 | 678 | 3072 |
| BST1 | 195 | 2589 | 437 | 2831 | 679 | 3073 |
| BST1 | 196 | 2590 | 438 | 2832 | 680 | 3074 |
| BST1 | 197 | 2591 | 439 | 2833 | 681 | 3075 |
| CD19 | 198 | 2592 | 440 | 2834 | 682 | 3076 |
| CD22 | 199 | 2593 | 441 | 2835 | 683 | 3077 |
| CD70 | 200 | 2594 | 442 | 2836 | 684 | 3078 |
| BCMA | 201 | 2595 | 443 | 2837 | 685 | 3079 |
| Her2 | 202 | 2596 | 444 | 2838 | 686 | 3080 |
| Her2 | 203 | 2597 | 445 | 2839 | 687 | 3081 |
| Her2 | 204 | 2598 | 446 | 2840 | 688 | 3082 |
| Her2 | 205 | 2599 | 447 | 2841 | 689 | 3083 |
| MSLN | 206 | 2600 | 448 | 2842 | 690 | 3084 |
| MSLN | 207 | 2601 | 449 | 2843 | 691 | 3085 |
| EGFRviii | 208 | 2602 | 450 | 2844 | 692 | 3086 |
| EGFRviii | 209 | 2603 | 451 | 2845 | 693 | 3087 |
| DLL3 | 210 | 2604 | 452 | 2846 | 694 | 3088 |
| DLL3 | 211 | 2605 | 453 | 2847 | 695 | 3089 |
| Nectin4 | 212 | 2606 | 454 | 2848 | 696 | 3090 |
| MSLN | 213 | 2607 | 455 | 2849 | 697 | 3091 |
| MSLN | 214 | 2608 | 456 | 2850 | 698 | 3092 |
| MSLN | 215 | 2609 | 457 | 2851 | 699 | 3093 |
| PRLR | 216 | 2610 | 458 | 2852 | 700 | 3094 |
| EMR2 | 217 | 2611 | 459 | 2853 | 701 | 3095 |
| CEA | 218 | 2612 | 460 | 2854 | 702 | 3096 |
| Her3 | 219 | 2613 | 461 | 2855 | 703 | 3097 |
| FOLR1 | 220 | 2614 | 462 | 2856 | 704 | 3098 |
| FOLR1 | 221 | 2615 | 463 | 2857 | 705 | 3099 |
| CLDN6 | 222 | 2616 | 464 | 2858 | 706 | 3100 |
| CLDN6 | 223 | 2617 | 465 | 2859 | 707 | 3101 |
| SLC34A2 | 224 | 2618 | 466 | 2860 | 708 | 3102 |
| CD22 | 225 | 2619 | 467 | 2861 | 709 | 3103 |
| CD22 | 226 | 2620 | 468 | 2862 | 710 | 3104 |
| BCMA | 227 | 2621 | 469 | 2863 | 711 | 3105 |
| CD22 | 228 | 2622 | 470 | 2864 | 712 | 3106 |
| CD19 | 229 | 2623 | 471 | 2865 | 713 | 3107 |
| BCMA | 230 | 2624 | 472 | 2866 | 714 | 3108 |
| MPL/TPO-R | 231 | 2625 | 473 | 2867 | 715 | 3109 |
| BAFFR | 232 | 2626 | 474 | 2868 | 716 | 3110 |
| BAFFR | 233 | 2627 | 475 | 2869 | 717 | 3111 |
| BAFFR | 234 | 2628 | 476 | 2870 | 718 | 3112 |
| BCMA | 235 | 2629 | 477 | 2871 | 719 | 3113 |
| BCMA | 236 | 2630 | 478 | 2872 | 720 | 3114 |
| BCMA | 237 | 2631 | 479 | 2873 | 721 | 3115 |
| BCMA | 238 | 2632 | 480 | 2874 | 722 | 3116 |
| BCMA | 239 | 2633 | 481 | 2875 | 723 | 3117 |
| BCMA | 240 | 2634 | 482 | 2876 | 724 | 3118 |
| BCMA | 241 | 2635 | 483 | 2877 | 725 | 3119 |
| BCMA | 242 | 2636 | 484 | 2878 | 726 | 3120 |
| BCMA | 243 | 2637 | 485 | 2879 | 727 | 3121 |
| BCMA | 244 | 2638 | 486 | 2880 | 728 | 3122 |
| BCMA | 245 | 2639 | 487 | 2881 | 729 | 3123 |
| BCMA | 246 | 2640 | 488 | 2882 | 730 | 3124 |
| BCMA | 247 | 2641 | 489 | 2883 | 731 | 3125 |
| BCMA | 248 | 2642 | 490 | 2884 | 732 | 3126 |
| ROR1 | 249 | 2643 | 491 | 2885 | 733 | 3127 |
| ROR1 | 250 | 2644 | 492 | 2886 | 734 | 3128 |
| ROR1 | 251 | 2645 | 493 | 2887 | 735 | 3129 |
| CD20 | 252 | 2646 | 494 | 2888 | 736 | 3130 |
| Her2 | 253 | 2647 | 495 | 2889 | 737 | 3131 |
| CD19 | 254 | 2648 | 496 | 2890 | 738 | 3132 |
| CEA | 255 | 2649 | 497 | 2891 | 739 | 3133 |
| Her2 | 256 | 2650 | 498 | 2892 | 740 | 3134 |
| Her2 | 257 | 2651 | 499 | 2893 | 741 | 3135 |
| TOSO | 258 | 2652 | 500 | 2894 | 742 | 3136 |
| CD30 | 259 | 2653 | 501 | 2895 | 743 | 3137 |
| CD229 | 260 | 2654 | 502 | 2896 | 744 | 3138 |
| CD229 | 261 | 2655 | 503 | 2897 | 745 | 3139 |
| CD229 | 262 | 2656 | 504 | 2898 | 746 | 3140 |
| CD229 | 263 | 2657 | 505 | 2899 | 747 | 3141 |
| EBV-gp350 | 264 | 2658 | 506 | 2900 | 748 | 3142 |
| EBV-gp350 | 265 | 2659 | 507 | 2901 | 749 | 3143 |
| INFL-NA | 266 | 2660 | 508 | 2902 | 750 | 3144 |
| EBV-LMP1 | 267 | 2661 | 509 | 2903 | 751 | 3145 |
| PSMA | 268 | 2662 | 510 | 2904 | 752 | 3146 |
| PSMA | 269 | 2663 | 511 | 2905 | 753 | 3147 |
| PSMA | 270 | 2664 | 512 | 2906 | 754 | 3148 |
| PSMA | 271 | 2665 | 513 | 2907 | 755 | 3149 |
| PSMA | 272 | 2666 | 514 | 2908 | 756 | 3150 |
| MUC1 | 273 | 2667 | 515 | 2909 | 757 | 3151 |
| MUC1 | 274 | 2668 | 516 | 2910 | 758 | 3152 |
| MUC1 | 275 | 2669 | 517 | 2911 | 759 | 3153 |
| gpA33 | 276 | 2670 | 518 | 2912 | 760 | 3154 |
| MSLN | 277 | 2671 | 519 | 2913 | 761 | 3155 |
| MSLN | 278 | 2672 | 520 | 2914 | 762 | 3156 |
| MSLN | 279 | 2673 | 521 | 2915 | 763 | 3157 |
| MSLN | 280 | 2674 | 522 | 2916 | 764 | 3158 |
| MSLN | 281 | 2675 | 523 | 2917 | 765 | 3159 |
| MSLN | 282 | 2676 | 524 | 2918 | 766 | 3160 |

TABLE 4

| | | SEQ | SEQ | | SEQ | SEQ |
|---|---|---|---|---|---|---|
| TARGET | Va/Vd | ID NO | ID NO | Vb/Vg | ID NO | ID NO |
| ANTIGEN | NAME | (DNA) | (PRT) | NAME | (DNA) | (PRT) |
| MR1 | MC7G5Va | 963 | 3357 | MC7G5Vb | 964 | 3358 |
| NY-ESO-1/ | NY-ESO- | 965 | 3359 | NY-ESO- | 966 | 3360 |
| HLA-A2 | 1-IG4-Va | | | 1-IG4-Vb | | |
| CMV-pp65/ | CMV- | 967 | 3361 | CMV- | 968 | 3362 |
| HLA-A2 | pp65-Va | | | pp65- | | |
| | | | | TCR1-Vb | | |
| | TCR-Vd2 | 969 | 3363 | TCR-Vg9 | 970 | 3364 |

TABLE 5

| Target Antigen | SEQ ID NO (DNA) | SEQ ID NO (PRT) |
|---|---|---|
| CEA | 816 | 3210 |
| CEA | 817 | 3211 |
| CD20 × CD22 | 818 | 3212 |
| CD20 | 819 | 3213 |
| PSMA | 820 | 3214 |
| PSMA | 821 | 3215 |
| PSMA | 822 | 3216 |
| PSMA | 823 | 3217 |
| CD38 | 824 | 3218 |
| CD38 | 825 | 3219 |
| CD38 | 826 | 3220 |
| BCMA | 827 | 3221 |
| BCMA | 828 | 3222 |
| CD22 × BCMA | 829 | 3223 |
| PSMA | 830 | 3224 |
| PSMA | 831 | 3225 |
| PSMA | 832 | 3226 |
| PSMA | 833 | 3227 |
| CD19 | 834 | 3228 |
| CD20 | 835 | 3229 |
| CD19 | 836 | 3230 |
| CD19 | 837 | 3231 |
| CD19 | 838 | 3232 |
| CD19 | 839 | 3233 |
| CD20 | 840 | 3234 |
| CD20 | 841 | 3235 |
| CD22 | 842 | 3236 |
| CD22 | 843 | 3237 |
| CD22 | 844 | 3238 |
| CD38 | 845 | 3239 |
| CD38 | 846 | 3240 |
| CD38 | 847 | 3241 |
| CD38 | 848 | 3242 |
| CD38 | 849 | 3243 |
| CEA | 850 | 3244 |
| CEA | 851 | 3245 |
| BCMA | 852 | 3246 |
| BCMA | 853 | 3247 |
| BCMA | 854 | 3248 |
| BCMA | 855 | 3249 |
| BCMA | 856 | 3250 |
| BCMA | 857 | 3251 |
| BCMA | 858 | 3252 |
| Muc16 | 859 | 3253 |
| Muc16 | 860 | 3254 |
| Muc16 | 861 | 3255 |
| IL13Ra2 | 862 | 3256 |
| IL13Ra2 | 863 | 3257 |
| Her2 | 864 | 3258 |
| Her2 | 865 | 3259 |
| Her2 | 866 | 3260 |
| Her3 | 867 | 3261 |
| Her3 | 868 | 3262 |
| CEA | 869 | 3263 |
| CEA | 870 | 3264 |
| EGFR | 871 | 3265 |
| EGFR | 872 | 3266 |
| cMet | 873 | 3267 |
| CXCR4 | 874 | 3268 |
| CXCR4 | 875 | 3269 |
| MSLN | 876 | 3270 |
| MSLN | 877 | 3271 |
| Albumin | 878 | 3272 |
| CD123 | 879 | 3273 |
| CD123 | 880 | 3274 |
| IL6R | 881 | 3275 |
| EGFR and CEA | 882 | 3276 |
| EGFR and CEA | 883 | 3277 |
| Her2 and Her2 | 884 | 3278 |
| Her2 and Her2 | 885 | 3279 |
| Her3 and Her2 | 886 | 3280 |
| cMET and Her3 | 887 | 3281 |
| MSLN | 888 | 3282 |
| BCMA | 889 | 3283 |
| BCMA | 890 | 3284 |
| BCMA | 891 | 3285 |
| CD38 | 892 | 3286 |
| BCMA | 893 | 3287 |
| CD38 and BCMA | 894 | 3288 |
| BCMA | 895 | 3289 |
| CD38 | 896 | 3290 |
| BCMA and CD38 | 897 | 3291 |
| CD19 | 898 | 3292 |
| CD20 | 899 | 3293 |
| CD19 and CD20 | 900 | 3294 |
| BCMA | 901 | 3295 |
| BCMA | 902 | 3296 |

TABLE 13
Ig like Linker domain

| | SEQ ID | SEQ ID |
|---|---|---|
| Name of fragment | NO (DNA) | NO (PRT) |
| IgCL | 1142 | 3536 |
| IgG1-CH1 | 1143 | 3537 |
| IgG1-CH1-DeltaC | 1144 | 3538 |
| IgG1-CH1-Hinge | 1145 | 3539 |
| IgG1-CH1-v2 | 1146 | 3540 |
| IgG1-CH1-DeltaC-v2 | 1147 | 3541 |
| IgG1-CH1-Hinge-v2 | 1148 | 3542 |
| IgG2-0C-CH1 | 1149 | 3543 |
| IgG2-IC-CHI1 | 1150 | 3544 |
| IgG3-CHI1 | 1151 | 3545 |
| IgG4-CHI1 | 1152 | 3546 |
| IgAI-CHI1 | 1153 | 3547 |
| IgA2-CHI1 | 1154 | 3548 |
| IgD-CHI1 | 1155 | 3549 |
| IgE-CHI1 | 1156 | 3550 |
| IgM-CHI1 | 1157 | 3551 |
| TCRa-wt-opt-6ECD (TCRa-Ig1) | 1158 | 3552 |
| TCRa-wt-opt-7ECD (TCRa-Ig2) | 1159 | 3553 |
| TCRb-wt-opt-6ECD (TCRb-Ig1) | 1160 | 3554 |
| TCRb-wt-opt-ECD-7ECD (TCRb-Ig2) | 1161 | 3555 |
| TCRg-opt-6ECD (TCRg-Ig1) | 1162 | 3556 |
| TCRg-opt-7ECD (TCRg-Ig2) | 1163 | 3557 |
| TCRd-opt-6ECD (TCRd-Ig1) | 1164 | 3558 |
| TCRd-opt-ECD-7ECD (TCRd-Ig2) | 1165 | 3559 |
| TCRb-wt-opt-8ECD (TCRb-Ig3) | 1166 | 3560 |
| TCRa-wt-opt-8ECD (TCRa-Ig4) | 1167 | 3561 |
| TCRa-Ig-Like-C1-Domain-6MD (TCRa-Ig3) | 1168 | 3562 |
| TCRa-Ig-Like-C1-Domain (TCRa-Ig5) | 1169 | 3563 |
| TCRb-Ig-Like-C1-Domain-6MD (TCRb-Ig4) | 1170 | 3564 |
| TCRb-Ig-Like-C1-Domain (TCRb-Ig5) | 1171 | 3565 |
| TCRg-Ig-Like-C1-Domain-6MD (TCRg-Ig3) | 1172 | 3566 |
| TCRg-Ig-Like-C1-Domain (TCRg-Ig4) | 1173 | 3567 |
| TCRd-Ig-Like-C1-Domain (TCRd-Ig3) | 1174 | 3568 |
| TCRd-Ig-Like-C1-Domain-6MD (TCRd-Ig4) | 1175 | 3569 |

TABLE 18
Miscellaneous Domains

| | SEQ ID | SEQ ID |
|---|---|---|
| Name of fragment | NO (DNA) | NO (PRT) |
| CD3z-cytosolic-domain | 1206 | 3600 |
| CD3z-cytosolic-domain | 1207 | 3601 |
| 4-1BB-cytosolic-domain | 1208 | 3602 |
| hCD8-Hinge-TM | 1209 | 3603 |
| hCD8-Hinge-TM-BBz | 1210 | 3604 |
| hCD8TM-Hinge-BB | 1211 | 3605 |
| CD28-Hinge-TM-cytosolic-domain | 1212 | 3606 |
| CD3d-ECDTMCP-opt2 | 1213 | 3607 |
| CD3eECDTMCP-opt2 | 1214 | 3608 |
| CD3g-ECDTMCP-opt2 | 1215 | 3609 |
| CD3zECDTMCP-opt2 | 1216 | 3610 |
| CD3zECDTMCP-opt | 1090 | 3484 |
| CD28-CP-opt | 1091 | 3485 |
| 41BB-CP-opt | 1092 | 3486 |
| CD3e-CP-opt | 1093 | 3487 |
| CD3zECDTM-opt2 | 1094 | 3488 |
| CD3zCP-opt2 | 1095 | 3489 |
| CD3zECDTMCP-opt2 | 1096 | 3490 |
| CD28-CP-opt2 | 1097 | 3491 |
| 41BB-CP-opt2 | 1098 | 3492 |

TABLE 25
Novel SAR fragments

| | SEQ ID | SEQ ID |
|---|---|---|
| Name of fragment | NO (DNA) | NO (PRT) |
| DAP10-opt1 | 1349 | 3743 |
| DAP10-opt2 | 1350 | 3744 |
| CD3z-ECDTM-opt1 | 1351 | 3745 |
| CD3z-ECDTM-opt2 | 1352 | 3746 |
| mutCD3z-ECDTM-opt1 | 1353 | 3747 |
| mutCD3z-ECDTM-opt2 | 1354 | 3748 |
| OX40-CP-opt1 | 1355 | 3749 |
| OX40-CP-opt2 | 1356 | 3750 |
| CD3z-ECDTM-OX40-opt1 | 1357 | 3751 |
| CD3z-ECDTM-OX40-opt2 | 1358 | 3752 |
| mutCD3z-ECDTM-OX40-opt1 | 1359 | 3753 |
| mutCD3z-ECDTM-OX40-opt2 | 1360 | 3754 |
| DAP12-SP-Bam-DAP12-ECDTMCP-opt1 | 1361 | 3755 |
| DAP12-ECDTMCP-opt1 | 1362 | 3756 |
| DAP12-SP-Kpn-DAP12-ECDTMCP-opt2 | 1363 | 3757 |
| DAP12-ECDTMCP-opt2 | 1364 | 3758 |
| DAP12-SP-Bam-DAP12-C35S-ECDTMCP-opt1 | 1365 | 3759 |
| DAP12-C35S-ECDTMCP-opt1 | 1366 | 3760 |
| DAP12-SP-Kpn-DAP12-C35S-ECDTMCP-opt2 | 1367 | 3761 |
| DAP12-C35S-ECDTMCP-opt2 | 1368 | 3762 |
| NKp30-ECDTMCP-opt1 | 1369 | 3763 |
| NKp30-Hinge-TMCP-opt1 | 1370 | 3764 |
| NKp30-D1-opt1 | 1371 | 3765 |
| NKp30-Hinge-opt1 | 1372 | 3766 |
| NKp30-TM-opt1 | 1373 | 3767 |
| NKp30-CP-opt1 | 1374 | 3768 |
| NKp30-ECDTMCP-opt2 | 1375 | 3769 |
| NKp30-Hinge-TM-CP-opt2 | 1376 | 3770 |
| NKp30-D1-opt2 | 1377 | 3771 |
| NKp30-Hinge-opt2 | 1378 | 3772 |
| NKp30-TM-opt2 | 1379 | 3773 |
| NKp30-CP-opt2 | 1380 | 3774 |
| NKp44SP-Kpn-EcoR1-Xho-NKp44-ECDTMCP-opt1 | 1381 | 3775 |
| NKp44-ECDTMCP-opt1 | 1382 | 3776 |
| NKp44-Hinge-TM-CP-opt1 | 1383 | 3777 |
| Nkp44-Ig-opt1 | 1384 | 3778 |
| NKp44-Hinge-opt1 | 1385 | 3779 |
| NKp44-TM-opt1 | 1386 | 3780 |
| NKp44-CP-opt1 | 1387 | 3781 |
| NKp44SP2-Bam-Bst-Mlu-NKp44-ECDTMCP-opt2 | 1388 | 3782 |
| NKp44-ECDTMCP-opt2 | 1389 | 3783 |
| NKp44-Hinge-TM-CP-opt2 | 1390 | 3784 |
| Nkp44-Ig-opt2 | 1391 | 3785 |
| NKp44-Hinge-opt2 | 1392 | 3786 |
| NKp44-TM-opt2 | 1393 | 3787 |
| NKp44-CP-opt2 | 1394 | 3788 |
| NKp46-ECDTMCP-opt1 | 1395 | 3789 |
| NKp46-Linker-Ig1-Hinge-TM-CP-opt1 | 1396 | 3790 |
| NKp46-Ig1-Hinge-TM-CP-opt1 | 1397 | 3791 |
| NKp46-Hinge-TM-CP-opt1 | 1398 | 3792 |
| NKp46-Ig1-opt1 | 1399 | 3793 |
| NKp46-Linker-opt1 | 1400 | 3794 |
| NKp46-Ig2-opt1 | 1401 | 3795 |
| NKp46-Hinge-opt1 | 1402 | 3796 |
| NKp46-TM-opt1 | 1403 | 3797 |
| NKp46-CP-opt1 | 1404 | 3798 |
| NKp46-ECDTMCP-opt2 | 1405 | 3799 |
| NKp46-Linker-Ig1-Hinge-TM-CP-opt2 | 1406 | 3800 |
| NKp46-Ig1-Hinge-TM-CP-opt2 | 1407 | 3801 |
| NKp46-Hinge-TM-CP-opt2 | 1408 | 3802 |
| NKp46-Ig1-opt2 | 1409 | 3803 |
| NKp46-Linker-opt2 | 1410 | 3804 |
| NKp46-Ig2-opt2 | 1411 | 3805 |
| NKp46-Hinge-opt2 | 1412 | 3806 |
| NKp46-TM-opt2 | 1413 | 3807 |
| NKp46-CP-opt2 | 1414 | 3808 |
| CD16A-F158V-FL-v1 | 1415 | 3809 |
| CD16A-F158V-FL-v2 | 1416 | 3810 |
| CD16A-F158V-S197P-FL-v3 | 1417 | 3811 |
| FcRy-SP1-Bam-FcRy-ECDTMCP-opt1 | 1418 | 3812 |
| FcRy-ECDTMCP-opt1 | 1419 | 3813 |
| FcRy-SP2-Kpn-FcRy-ECDTMCP-opt2 | 1420 | 3814 |
| FcRy-ECDTMCP-opt2 | 1421 | 3815 |
| FcRy-SP1-Bam-FcRy-C24S-ECDTMCP-opt1 | 1422 | 3816 |
| FcRy-C24S-ECDTMCP-opt1 | 1423 | 3817 |
| FcRy-SP2-Kpn-FcRy-C24S-ECDTMCP-opt2 | 1424 | 3818 |
| FcRy-C24S-ECDTMCP-opt2 | 1425 | 3819 |
| mutCD3z-ECDTM-2B4CP-opt1 | 1426 | 3820 |
| 2B4CP-opt1 | 1427 | 3821 |
| mutCD3z-ECDTM-2B4CP-opt2 | 1428 | 3822 |
| 2B4CP-opt2 | 1429 | 3823 |
| CD8-hinge-NKG2D-TM-2B4CP-opt1 | 1430 | 3824 |
| CD8-Hinge-opt1 | 1431 | 3825 |
| NKG2D-TM-opt1 | 1432 | 3826 |
| 2B4CP-opt1 | 1433 | 3827 |
| CD8-hinge-NKG2D-TM-2B4CP-opt-2 | 1434 | 3828 |
| CD8-Hinge-opt2 | 1435 | 3829 |
| NKG2D-TM-opt2 | 1436 | 3830 |
| 2B4CP-opt2 | 1437 | 3831 |
| mutCD8-hinge-NKG2D-TM-2B4CP-opt-1 | 1438 | 3832 |
| mutCD8-hinge-opt-1 | 1439 | 3833 |
| mutCD8-hinge-NKG2D-TM-2B4CP-opt-2 | 1440 | 3834 |
| mutCD8-hinge-opt-2 | 1441 | 3835 |
| CD16A-F158V-D1-v1 | 1442 | 3836 |
| CD16A-F158V-D2-v1 | 1443 | 3837 |
| CD16A-F158V-D1-D2-linker-v1 | 1444 | 3838 |
| CD16A-F158V-D2-Minus-Linker-v1 | 1445 | 3839 |
| CD16A-F158V-Hinge-v1 | 1446 | 3840 |
| CD16A-F158V-TM-v1 | 1447 | 3841 |
| CD16A-F158V-cP-v1 | 1448 | 3842 |
| CD16-F158V-FL-TMCP-v1 | 1449 | 3843 |
| CD16-F158V-D2TMCP-v1 | 1450 | 3844 |
| CD16-F158V-Hinge-TM-CP-v1 | 1451 | 3845 |
| CD16A-F158V-D1-v3 | 1452 | 3846 |
| CD16A-F158V-D2-v3 | 1453 | 3847 |
| CD16A-F158V-D1-D2-linker-v3 | 1454 | 3848 |
| CD16A-F158V-D2-Minus-Linker-v3 | 1455 | 3849 |
| CD16A-F158V-Hinge-v3 | 1456 | 3850 |
| CD16A-F158V-TM-v3 | 1457 | 3851 |
| CD16A-F158V-cP-v3 | 1458 | 3852 |
| CD16-F158V-S197P-FL-TMCP-v3 | 1459 | 3853 |
| CD16-F158V-S197P-D2TMCP-v3 | 1460 | 3854 |
| CD16-F158V-S197P-Hinge-TM-CP-v3 | 1461 | 3855 |
| CD16A-F158V-D1-v2 | 1462 | 3856 |
| CD16A-F158V-D2-v2 | 1463 | 3857 |
| CD16A-F158V-D1-D2-linker-v2 | 1464 | 3858 |
| CD16A-F158V-D2-Minus-Linker-v2 | 1465 | 3859 |
| CD16A-F158V-Hinge-v2 | 1466 | 3860 |
| CD16A-F158V-TM-v2 | 1467 | 3861 |
| CD16A-F158V-CP-v2 | 1468 | 3862 |
| CD16-F158V-FL-TMCP-v2 | 1469 | 3863 |
| CD16-F158V-D2TMCP-v2 | 1470 | 3864 |
| CD16-F158V-Hinge-TM-CP-v2 | 1471 | 3865 |
| DNAM1-Hinge-TMCP-opt1 | 1472 | 3866 |
| DNAM1-Hinge-opt1 | 1473 | 3867 |
| DNAM1-TM-opt1 | 1474 | 3868 |
| DNAM1-CP-opt1 | 1475 | 3869 |
| DNAM1-Hinge-TMCP-opt2 | 1476 | 3870 |
| DNAM1-Hinge-opt2 | 1477 | 3871 |
| DNAM1-TM-opt2 | 1478 | 3872 |
| DNAM1-CP-opt2 | 1479 | 3873 |
| DNAM1-opt1 | 1480 | 3874 |
| DNAM1-Ig-Hinge-TM-CP-opt1 | 1481 | 3875 |
| DNAM1-opt2 | 1482 | 3876 |
| DNAM1-Ig-Hinge-TM-CP-opt2 | 1483 | 3877 |
| IL2Rb-hinge-TMCP | 1484 | 3878 |
| IL2Ry-Hinge-TMCP | 1485 | 3879 |
| 41BB-ECDTMCP-opt1 | 1573 | 3967 |
| 41BB-ECDTMCP-opt2 | 1574 | 3968 |
| CD28-ECDTMCP-opt1 | 1575 | 3969 |
| CD28-ECDTMCP-opt2 | 1576 | 3970 |
| OX40-ECDTMCP-opt1 | 1577 | 3971 |
| OX40-ECDTMCP-opt2 | 1578 | 3972 |
| 2B4-ECDTMCP-opt1 | 1579 | 3973 |
| 2B4-ECDTMCP-opt2 | 1580 | 3974 |
| CD32-ECDTMCP-opt1 | 1581 | 3975 |
| CD32-ECDTMCP-opt2 | 1582 | 3976 |
| CD64-ECDTMCP-opt1 | 1583 | 3977 |
| CD64-ECDTMCP-opt2 | 1584 | 3978 |

TABLE 34
SARs

| | SEQ ID | SEQ ID |
|---|---|---|
| Name of fragment | NO (DNA) | NO (PRT) |
| CD8SP-CD19-hu-mROO5-1-vL-Xho-IgCL-DAP10-opt1-F-P2A- | 2275 | 4669 |
| Spe-IgSP-Bst-CD19-hu-mROO5-1-vH-Mlu-Ig1CH1-DAP10-opt2- | | |
| F-F2A-Xba-PAC | | |
| CD8SP-hCD19-EUK-5-13-vL-IgCL-Bam-CD3zECDTMCP-opt-F- | 2276 | 4670 |
| P2A-Spe-SP-Bst-hCD19-EUK-5-13-vH-IgG1-CH1-Kpn-CD16A- | | |
| Hinge-TM-CP-F158V-F-P2A-Xba-PAC | | |
| CD8SP-hCD19-EUK-5-13-vL-IgCL-Bam-CD3zECDTMCP-opt-F- | 2277 | 4671 |
| P2A-Spe-SP-Bst-hCD19-EUK-5-13-vH-IgG1-CH1-KPN- | | |
| CD3zECDTMCP-opt2-F-F2A-Xba-PAC | | |
| CD8SP-CD19-hu-mROO5-1-vL-xho-IgCL-KPN-NC-D2GKN- | 2278 | 4672 |
| ECD-TM-CP-opt1-F-P2A-Spe-Bst-CD19-hu-mROO5-1-vH-Mlu- | | |
| Ig1CH1-Bam-NC-D2GKN-ECD-TM-CP-opt2-F-F2A-Xba-PAC | | |
| CD8SP-CD19-hu-mROO5-1-Mlu-CD8-hing-NKG2D-TM-2B4z-F- | 2279 | 4673 |
| P2A-CD3z-Xba-P2A-Bam-SynthK13-xho-Flag-Spe-F-P3A-Nde- | | |
| PAC | | |
| CD8SP-CD19-hu-mROO5-1-vL-xho-IgCL-KPN-NC-D2GKN- | 2280 | 4674 |
| ECD-TM-CP-opt1-F-P2A-Spe-Bst-CD19-hu-mROO5-1-vH-Mlu- | | |
| Hinge-TM-CP-NKp46-opt2-F-F2A-Xba-PAC | | |
| CD8SP-CD19-hu-mROO5-1-vL-Xho-NKp46-Hinge-TM-CP-opt1- | 2281 | 4675 |
| F-P2A-Spe-IgSP-Bst-CD19-hu-mROO5-1-vH-Mlu-NKp46-Hinge- | | |
| TM-CP-opt2-F-F2A-Xba-PAC | | |
| CD8SP-CD19-hu-mROO5-1-vL-Xho-CD16A-Hinge-TM-CP- | 2282 | 4676 |
| F158V-F-P2A-Spe-Bst-CD19-hu-mROO5-1-vH-Mlu-CD16A- | | |
| F158V-Hinge-TM-CP-v2-F-F2A-Xba-PAC | | |
| CD8SP-Sph-BCMA-FHVH93-Kpn-G4S-EcoR1-CD16A-F158V- | 2283 | 4677 |
| FL-F-P2A-Spe-IgHSP-Apa-CD20-VHH-USC1-2HC2D6-Bam- | | |
| G4S-Bst-CD16A-F158V-FL-v2-F2A-Xba-PAC | | |
| CD8SP-hu-mROO5-1-vL-xho-hTCRbECD-Bam- | 2287 | 4681 |
| CD3zECDTMCP-opt-F-P2A-SP-hu-mROO5-1-vH-Mlu- | | |
| hTCRaECD-Kpn-CD3zECDTMCP-opt2-F-F2A-PAC | | |
| CD8SP-hu-mROO5-1-vL-Xho-CD3zECDTMCP-opt-F-P2A-Spe- | 2288 | 4682 |
| SP-Bst-hu-mROO5-1-vH-Mlu-CD3zECDTMCP-opt2-F-F2A-PAC | | |
| CD8SP-hu-mROO5-1-vL-xho-IgCL-Bam-CD3zECDTMCP-opt-F- | 2289 | 4683 |
| P2A-SP-hu-mROO5-1-vH-Mlu-IgG1-CH1-Kpn-CD3zECDTMCP- | | |
| opt2-F-F2A-PAC | | |
| CD8-hCD19-EUK5-13-vL-IgCL-Bam-CD3zECDTMCP-opt-F- | 2290 | 4684 |
| P2A-Spe-SP-Bst-hCD19-EUK5-13-vH-IgG1-CH1-KPN- | | |
| CD3zECDTMCP-opt2-F-F2A-Xba-PAC | | |
| CD8-hCD19-EUK5-13-vL-IgCL-Xho-CD3zECDTMCP-opt-F- | 2291 | 4685 |
| P2A-Spe-SP-Bst-hCD19-EUK5-13-vH-IgG1-CH1-Mlu- | | |
| CD3zECDTMCP-opt2-F-F2A-PAC | | |
| CD8SP-hCD19-EUK-5-13-vL-IgCL-Xho-NKp30-ECDTMCP- | 2292 | 4686 |
| opt1-F-P2A-Spe-SP-Bst-hCD19-EUK-5-13-vH-IgG1-CH1-Mlu- | | |
| NKp30-ECDTMCP-opt2-F-F2A-Xba-PAC | | |
| CD8SP-CD19-hu-mROO5-1-vL-Xho-NKp30-ECDTMCP-opt1-F- | 2293 | 4687 |
| P2A-Spe-SP-Bst-CD19-hu-mROO5-1-vH-Mlu-NKp30- | | |
| ECDTMCP-opt2-F-F2A-Xba-PAC | | |
| CD8SP-Sph-BCMA-FHVH93-Kpn-G4S-EcoR1-NKp44-Hinge- | 2296 | 4690 |
| ECDTMCP-opt1-F-P2A-Spe-IgHSP-Apa-CD20-VHH-USC1- | | |
| 2HC2D6-Bam-G4S-Bst-NKp44-Hinge-ECDTMCP-opt2-F2A- | | |
| Xba-PAC | | |
| CD8SP-Sph-BCMA-FHVH93-Kpn-G4S-EcoR1-Xho-NKp30- | 2297 | 4691 |
| ECDTMCP-opt1-F-P2A-Spe-IgHSP-Apa-CD20-VHH-USC1- | | |
| 2HC2D6-Bam-G4S-Bst-Mlu-NKp30-ECDTMCP-opt2-F-F2A- | | |
| Xba-PAC | | |
| CD8-hCD19-EUK-5-13-vL-IgCL-Xho-NKp44-ECDTMCP-opt1- | 2298 | 4692 |
| F-P2A-Spe-SP-Bst-hCD19-EUK-5-13-vH-IgG1-CH1-Mlu-NKp44- | | |
| ECDTMCP-opt2-F-F2A-Xba-PAC | | |
| CD8SP-CD19-hu-mROO5-1-vL-Xho-NKp44-ECDTMCP-opt1-F- | 2299 | 4693 |
| P2A-Spe-IgSP-Bst-CD19-hu-mROO5-1-vH-Mlu-NKp44- | | |
| ECDTMCP-opt2-F-F2A-Xba-PAC | | |
| CD8SP-CD19-hu-mROO5-1-vL-Xho-NKp44-Hinge-TMCP-opt1- | 2300 | 4694 |
| F-P2A-Spe-IgSP-Bst-CD19-hu-mROO5-1-vH-Mlu-NKp44-Hinge- | | |
| TMCP-opt2-F-F2A-Xba-PAC | | |
| CD8SP-hu-mROO5-1-vL-xho-IgCL-Bam-NKp30-ECDTMCP- | 2301 | 4695 |
| opt1-F-P2A-SP-hu-mROO5-1-vH-Mlu-IgG1-CH1-Kpn- | | |
| CD3zECDTMCP-opt2-F-F2A-PAC | | |
| CD8SP-hu-mROO5-1-vL-xho-IgCL-Bam-NKp30-ECDTMCP- | 2302 | 4696 |
| opt1-F-P2A-SP-hu-mROO5-1-vH-Mlu-IgG1-CH1-Kpn-NKp30- | | |
| ECDTMCP-opt2-F-F2A-PAC | | |
| CD8SP-hu-mROO5-1-scFv-Myc-CD8TM-28z | 2303 | 4697 |
| CD8SP-hu-mROO5-1-scFv-Myc-CD8TM-BBz | 2304 | 4698 |
| CD8SP-hu-mROO5-1-vL-[hTCRb-S57C]-F-P2A-SP-hu-mROO5- | 2305 | 4699 |
| 1-vH-[hTCRa-T48C] | | |
| CD8SP-hu-mROO5-1-vL-IgCL-Bam-CD3zECDTMCP-opt-F- | 2306 | 4700 |
| P2A-Spe-SP-Bst-hu-mROO5-1-vH-IgG1-CH1-KPN- | | |
| CD3zECDTMCP | | |
| CD8SP-hu-mROO5-1-vL-CD3zECDTMCP-opt-F-P2A-Spe-SP- | 2307 | 4701 |
| Bst-hu-mROO5-1-vH-Mlu-CD3zECDTMCP-opt2 | | |
| CD8SP-hu-mROO5-1-vL-[hTCRbECD-Bam-CD3zECDTMCP- | 2308 | 4702 |
| opt]-F-P2A-SP-hu-mROO5-1-vH-[hTCRaECD-Kpn- | | |
| CD3zECDTMCP-opt2] | | |
| CD8SP-hu-mROO5-1-vL-[IgCL-TCRg-6MD]-F-P2A-SP-hu- | 2309 | 4703 |
| mROO5-1-vH-[IgG1-CH1-TCRd-6MD] | | |
| CD8SP-hu-mROO5-1-vL-[IgCL-TCRb-wt-opt2-6MD]-F-P2A-SP- | 2310 | 4704 |
| hu-mROO5-1-vH-[IgG1-CH1-TCRa-wt-op2-6MD] | | |
| CD8SP-hu-mROO5-1-scFv-CD3e-ECDTMCP-opt2 | 2311 | 4705 |
| CD8SP-hCD19-EUK-5-13-vL-IgCL-Bam-NKp46-Hinge-TM-CP- | 2312 | 4706 |
| opt2-F-F2A-Spe-SP-Bst-hCD19-EUK-5-13-vH-IgG1-CH1-Kpn- | | |
| NKp46-Hinge-TM-CP-opt1-F-P2A-Xba-PAC | | |
| CD8SP-hCD19-EUK-5-13-vL-IgCL-Bam-DAP10-opt1-Spe- | 2313 | 4707 |
| CD3zCP-opt1-F-P2A-dSPE-IgSP-Bst-hCD19-EUK-5-13-vH-IgG1- | | |
| CH1-Kpn-DAP10-opt2-Xba-CD3zCP-opt2-F-F2A-dXba-Nde-PAC | | |
| CD8SP-Sph-BCMA-FHVH93-Kpn-G4S-EcoR1-CD16A-F158A- | 2314 | 4708 |
| FL-F-P2A-Spe-IgHSP-Apa-CD20-VHH-USC1-2HC2D6-Bam- | | |
| G4S-Bst-CD19-hu-mROO5-1-vH-Mlu-[hTCRa-T48C-opt]-F-F2A- | | |
| Xba-PAC | | |
| CD8SP-Sph-BCMA-FHVH93-Kpn-G4S-EcoR1-CD16A-F158A- | 2315 | 4709 |
| D2TMCP-v1-F-P2A-Spe-IgHSP-Apa-CD20-VHH-USC1- | | |
| 2HC2D6-Bam-G4S-Bst-CD19-hu-mROO5-1-vH-Mlu-[hTCRa- | | |
| T48C-opt]-F-F2A-Xba-PAC | | |
| CD8SP-Sph-BCMA-FHVH93-Kpn-CD16-V158A-D2TMCP-v1-F- | 2316 | 4710 |
| P2A-Spe-IgHSP-Apa-CD20-VHH-USC1-2HC2D6-Bam-G4S-Bst- | | |
| CD19-hu-mROO5-1-vH-Mlu-[hTCRa-T48C-opt]-F-F2A-Xba-PAC | | |
| CD8SP-Sph-BCMA-FHVH93-Kpn-G4S-EcoR1-CD16-V158A- | 2317 | 4711 |
| D2TMCP-v1-F-P2A-SpeXba-PAC | | |
| IgHSP-Apa-CD20-VHH-USC1-2HC2D6-Bam-G4S-Bst-CD16A- | 2318 | 4712 |
| F158V-FL-v2-F2A-Xba-PAC | | |
| CD8SP-CD19-hu-mROO5-1-vL-Xho-NKp30-ECDTMCP-opt1-F- | 2319 | 4713 |
| P2A-Spe-IgSP-Bst-CD19-hu-mROO5-1-vH-Mlu-NKp46-Hinge- | | |
| TM-CP-opt2-F-F2A-Xba-PAC | | |
| CD8SP-Sph-BCMA-FHVH93-Kpn-G4S-EcoR1-NKp44-Hinge- | 2320 | 4714 |
| ECDTMCP-opt1-F-P2A-Spe-IgHSP-Apa-CD20-VHH-USC1- | | |
| 2HC2D6-Bam-G4S-Bst-CD16A-v158-FL-v2-F2A-Xba-PAC | | |
| CD8-hCD19-EUK-5-13-vL-IgCL-Xho-NKp44-ECDTMCP-opt1- | 2321 | 4715 |
| F-P2A-Spe-SP-Bst-hCD19-EUK-5-13-vH-IgG1-CH1-Mlu- | | |
| CD3zECDTMCP-opt2-F-F2A-PAC | | |
| CD8SP-Sph-BCMA-FHVH93-Kpn-CD16-V158A-D2TMCP-v1-F- | 2322 | 4716 |
| P2A-Spe-IgHSP-Apa-CD20-VHH-USC1-2HC2D6-Bam-CD16- | | |
| F158V-S197P-D2TMCP-v3-F-F2A-Xba-PAC | | |
| CD8-hCD19-EUK-5-13-vL-IgCL-Xho-NKp30-Hinge-TMCP-opt1- | 2323 | 4717 |
| F-P2A-Spe-SP-Bst-hCD19-EUK-5-13-vH-IgG1-CH1-Mlu- | | |
| CD3zECDTMCP-opt2-F-F2A-PAC | | |
| CD8-hCD19-EUK-5-13-vL-IgCL-Xho-NKp44-Hinge-TMCP-opt1- | 2324 | 4718 |
| F-P2A-Spe-SP-Bst-hCD19-EUK-5-13-vH-IgG1-CH1-Mlu- | | |
| CD3zECDTMCP-opt2-F-F2A-PAC | | |
| CD8SP-Sph-NKp30-Ig-Hinge-opt1-Xho-[hTCRb-S57C]-F-P2A- | 2325 | 4719 |
| Spe-IgHSP-Apa-CD20-VHH-USC1-2HC2D6-Bam-G4S-Bst- | | |
| CD19-hu-mROO5-1-vH-Mlu-[hTCRa-T48C-opt]-F-F2A-Xba-PAC | | |
| hCD19-EUK-5-13-vL-IgCL-Bam-CD8-hinge-NKG2D-TM-2B4- | 2326 | 4720 |
| CP-opt-1-Spe-CD3zCP-opt1-F-P2A-dSPE-IgSP-Bst-hCD19-EUK- | | |
| 5-13-vH-IgG1-CH1-Kpn-DAP10-opt2-Xba-CD3zCP-opt2-F-F2A- | | |
| dXba-Nde-PAC | | |
| CD8SP-CD19-hu-mROO5-1-vL-Xho-NKp44-ECDTMCP-opt1-F- | 2327 | 4721 |
| P2A-Spe-IgSP-Bst-CD19-hu-mROO5-1-vH-Mlu-NKp46-Hinge- | | |
| TM-CP-opt2-F-F2A-Xba-PAC | | |
| CD8SP-CD20-VHH-USC1-CD64-ECD-opt1-CD16A-TMCP-v3 | 2328 | 4722 |

TABLE 40
Miscellaneous Constructs

| | SEQ ID | SEQ ID |
|---|---|---|
| Name of fragment | NO (DNA) | NO (PRT) |
| CD8SP-BCMA-FHVH93-GS-ULBP2R | 5131 | 5431 |
| CD8SP-BCMA-FHVH93-GS-ULBP2-S3 | 5132 | 5432 |
| | 5133 | 5433 |
| CD8SP-FMC63-vL-[hTCRbECD-Bam-CD3zECDTMCP-opt]-F- | 5134 | 5434 |
| P2A-SP-FMC63-vH-[hTCRaECD-Kpn-CD3zECDTMCP-opt2]-F- | | |
| F2A-PAC | | |
| CD8SP-FMC63-vL-[hTCRb-KAC-ECD-Bam-CD3zECDTMCP- | 5135 | 5435 |
| opt]-F-P2A-SP-FMC63-vH-[hTCRa-CSDVP-ECD-Kpn- | | |
| CD3zECDTMCP-opt2]-F-F2A-PAC | | |
| CD8SP-FMC63-vL-V5-[hTCRbECD-Bam-CD3zECDTMCP-opt]- | 5136 | 5436 |
| F-P2A-SP-FMC63-vH-Myc-[hTCRaECD-Kpn-CD3zECDTM-28z- | | |
| opt2]-F-F2A-PAC | | |
| CD8SP-FMC63-vL-V5-[hTCRbECD-Bam-CD3zECDTM-BBz- | 5137 | 5437 |
| opt]-F-P2A-SP-FMC63-vH-Myc4-[hTCRaECD-Kpn- | | |
| CD3zECDTM-BBz-opt2]-F-F2A-PAC | | |
| CD8SP-FMC63-vL-V5-[hTCRbECD-Bam-CD3zECDTM-28z- | 5138 | 5438 |
| opt]-F-P2A-SP-FMC63-vH-Myc-[hTCRaECD-Kpn- | | |
| CD3zECDTM-28z-opt2]-F-F2A-PAC | | |
| CD8SP-CD19-hu-mROO5-vL-[hTCRb-KAC-ECD-Bam- | 5139 | 5439 |
| CD3zECDTMCP-opt]-F-F2A-SP-CD19-hu-mROO5-vH-[hTCRa- | | |
| CSDVP-ECD-Kpn-CD3zECDTMCP-opt2]-F-F2A-PAC | | |
| CD8SP-CD19-hu-mROO5-1-scFv-Myc-BBz-T2A-PAC | 5140 | 5440 |
| CD8SP-FMC63-scFv-Myc-BBz-T2A-PAC | 5141 | 5441 |

TABLE 43

| | SEQ ID | SEQ ID |
|---|---|---|
| Name of fragment | NO (DNA) | NO (PRT) |
| Synth-IL2-Nde-tBCMA-L244 | 7117 | 7810 |
| IgH-SP1-tBCMA-L244 | 7118 | 7811 |
| HSV1-Tk | 7119 | 7812 |
| Turkey-HSV2-Tk | 7120 | 7813 |
| MYR-FRB-FKBP-Nde-FADD-DED | 7121 | 7814 |
| MYR | 7122 | 7815 |
| FADD-DED | 7124 | 7816 |
| NKG2A-opt1 | 7125 | 7817 |
| CD94 | 7126 | 7818 |
| NKG2C | 7127 | 7819 |
| NKG2E | 7128 | 7820 |
| NKG2H | 7129 | 7821 |
| NKG2F | 7130 | 7822 |
| CD28-Hinge-TM | 7131 | 7823 |
| CD28-Hinge | 7132 | 7824 |
| hIL2-Mlu-SynthCD8TM | 7133 | 7825 |
| Synth-IL2m10-1-R38A-F42K-Q126A-Syth-CD28TM | 7134 | 7826 |
| Synth-IL2m4-R38E-F42A-SythCD28TM | 7135 | 7827 |
| Synth-IL2m4-1-R38E-F42A-I92A-SythCD28TM | 7136 | 7828 |
| Synth-IL2v-Roche-T3A-F42A-Y45A-L72G-C125A- | 7137 | 7829 |
| SythCD28TM | | |
| Synth-hIL15-E64K-SythCD28TM | 7138 | 7830 |
| Synth-hIL15-D8S-SythCD28TM | 7139 | 7831 |
| Synth-hIL15-L69R-SythCD28TM | 7140 | 7832 |
| Synth-IL2 | 7141 | 7833 |
| Synth-IL2m10-1-R38A-F42K-Q126A | 7142 | 7834 |
| Synth-IL2m4-R38E-F42A | 7143 | 7835 |
| Synth-IL2m4-1-R38E-F42A-I92A | 7144 | 7836 |
| Synth-IL2v-Roche-T3A-F42A-Y45A-L72G-C125A | 7145 | 7837 |
| hIL15 | 7146 | 7838 |
| hIL15-E64K | 7147 | 7839 |
| hIL15-D8S | 7148 | 7840 |
| hIL15-L69R | 7149 | 7841 |
| CD8SP-IL18 | 7150 | 7842 |
| Synth-IL2-Nde-tBCMA-L244ter | 7151 | 7843 |
| Synth-IL2m10-1-R38A-F42K-Q126A-Nde-tBCMA- | 7152 | 7844 |
| L244ter | | |
| Synth-IL2m4-R38E-F42A-Nde-tBCMA-L244ter | 7153 | 7845 |
| Synth-IL2m4-1-R38E-F42A-I92A-Nde-tBCMA-L244ter | 7154 | 7846 |
| Synth-IL2v-Roche-T3A-F42A-Y45A-L72G-C125A-Nde- | 7155 | 7847 |
| tBCMA-L244ter | | |
| Synth-IL15-Nde-tBCMA-L244ter | 7156 | 7848 |
| hIL15-E64K-Nde-tBCMA-L244ter | 7157 | 7849 |
| IL2-CD30-ECDTM | 7158 | 7850 |
| Nkp80 | 7159 | 7851 |

TABLE 44

| | SEQ ID | SEQ ID |
|---|---|---|
| Name of fragment | NO (DNA) | NO (PRT) |
| IgSP-Apa-CD20-USC1-vHH-2HCD26-G4Sx3v2-hu- | 7160 | 7852 |
| mROO5-1-scFv-CD16-F158V-S197P-FL-TMCP-v3 | | |
| IgSP-Apa-CD20-USC1-vHH-2HCD26-G4Sx3v2-hu- | 7161 | 7853 |
| mROO5-1-scFv-NKp30-ECDTMCP-opt1 | | |
| IgSP-Apa-CD20-USC1-vHH-2HCD26-G4Sx3v2-hu- | 7162 | 7854 |
| mROO5-1-scFv-NKp44-ECDTMCP-opt1 | | |
| IgSP-Apa-CD20-USC1-vHH-2HCD26-G4Sx3v2-hu- | 7163 | 7855 |
| mROO5-1-scFv-NKp46-ECDTMCP-opt1 | | |
| IgSP-Apa-CD20-USC1-vHH-2HCD26-G4Sx3v2-hu- | 7164 | 7856 |
| mROO5-1-scFv-CD28-Hinge-CD16-F158V-S197P-Hinge- | | |
| TM-CP-v3 | | |
| IgSP-Apa-CD20-USC1-vHH-2HCD26-G4Sx3v2-hu- | 7165 | 7857 |
| mROO5-1-scFv-CD28-Hinge-NKp30-Hinge-TMCP-opt1 | | |
| IgSP-Apa-CD20-USC1-vHH-2HCD26-G4Sx3v2-hu- | 7166 | 7858 |
| mROO5-1-scFv-CD28-Hinge-NKp44Hinge-TMCP-opt1 | | |
| IgSP-Apa-CD20-USC1-vHH-2HCD26-G4Sx3v2-hu- | 7167 | 7859 |
| mROO5-1-scFv-CD28-Hinge-NKp46-Hinge-TMCP-opt1 | | |
| IgSP-Apa-CD20-USC1-vHH-2HCD26-G4Sx3v2-hu- | 7168 | 7860 |
| mROO5-1-scFv-CD28-Hinge-DAP10-Hinge-TM-CP | | |
| IgSP-Apa-CD20-USC1-vHH-2HCD26-G4Sx3v2-hu- | 7169 | 7861 |
| mROO5-1-scFv-CD28-Hinge-CD3z-ECDTMCP | | |
| IgSP-Apa-CD20-USC1-vHH-2HCD26-G4Sx3v2-hu- | 7170 | 7862 |
| mROO5-1-scFv-CD28-Hinge-DNAM1-Hinge-TMCP | | |

TABLE 46

| | SEQ ID | SEQ ID |
|---|---|---|
| Name of fragment | NO (DNA) | NO (PRT) |
| CD8SP-BCMA-FHVH93-G4S-CD16A-V158-FL-F-P2A- | 7601 | 8293 |
| PAC | | |
| CD20-Ubli-NKp44-ECDTMCP-opt2-F-F2A-PAC | 7602 | 8294 |
| BCMA-J6M0-NKp44-ECDTMCP-opt2-F-F2A-PAC | 7603 | 8295 |
| CD22-h10F4v2-NKp44-ECDTMCP-opt2-F-F2A-PAC | 7604 | 8296 |
| CD8SP-BCMA917-vHH-E59D-NKp44-ECDTMCP-opt2- | 7605 | 8297 |
| F-F2A-PAC | | |
| CD8SP-PSMA-USC76-chVH-NKp44-ECDTMCP-opt2-F- | 7606 | 8298 |
| F2A-PAC | | |
| CD8SP-CD19-hu-mROO5-1-scFv-NKp30-ECDTMCP- | 7607 | 8299 |
| opt2-F-F2A-PAC | | |
| FMC64-NKp30-ECDTMCP-opt2-F-F2A-PAC | 7608 | 8300 |
| CD20-2F2-NKp30-ECDTMCP-opt2-F-F2A-PAC | 7609 | 8301 |
| CD8SP-Hul61-2-NKp30-ECDTMCP-opt2-F-F2A-PAC | 7610 | 8302 |
| BCMA-J6M0-NKp30-ECDTMCP-opt2-F-F2A-PAC | 7611 | 8303 |
| CD22-h10F4v2-NKp30-ECDTMCP-opt2-F-F2A-PA | 7612 | 8304 |
| CD8SP-hu-HA22-1-NKp30-ECDTMCP-opt2-F-F2A-PAC | 7613 | 8305 |
| CD8SP-CD20-VHH-USC1-2HC2D6-NKp30-ECDTMCP- | 7614 | 8306 |
| opt2-F-F2A-PAC | | |
| CD8SP-CD38-331-vHH-D64E-NKp30-ECDTMCP-opt2- | 7615 | 8307 |
| F-F2A-PAC | | |
| CD8SP-BCMA917-vHH-E59D-NKp30-ECDTMCP-opt2- | 7616 | 8308 |
| F-F2A-PAC | | |
| CD8SP-PSMA-USC76-chVH-NKp30-ECDTMCP-opt2-F- | 7617 | 8309 |
| F2A-PAC | | |
| CD8SP-BCMA-FHVH93-G4S-NKp46-opt1-F-P2A::Xba- | 7618 | 8310 |
| PAC | | |
| CD19-hu-mROO5-1-vL-huTCRg-F-P2A-SP-CD19-hu- | 7619 | 8311 |
| mROO5-1-vH-huTCRd-d2-F-F2A-PAC | | |
| CD19-hu-mROO5-1-vL-huTCRg-d1-F-P2A-SP-CD19-hu- | 7620 | 8312 |
| mROO5-1-vH-huTCRd-d2-F-F2A-PAC | | |
| CD19-hu-mROO5-1-vL-huTCRg-d2-F-P2A-SP-CD19-hu- | 7621 | 8313 |
| mROO5-1-vH-huTCRd-d2-F-F2A-PAC | | |
| CD19-hu-mROO5-1-vL-huTCRg-d8-F-P2A-SP-CD19-hu- | 7622 | 8314 |
| mROO5-1-vH-huTCRd-d2-F-F2A-PAC | | |
| CD19-hu-mROO5-1-vL-huTCRg-d11-F-P2A-CD19-hu- | 7623 | 8315 |
| mROO5-1-vH-huTCRd-d2-F-F2A-PAC | | |
| CD19-hu-mROO5-1-vL-huTCRg-d16-F-P2A-CD19-hu- | 7624 | 8316 |
| mROO5-1-vH-huTCRd-d2-F-F2A-PAC | | |
| CD19-hu-mROO5-1-vL-huTCRg-d21-F-P2A-CD19-hu- | 7625 | 8317 |
| mROO5-1-vH-huTCRd-d2-F-F2A-PAC | | |
| CD8SP-BCMA-FHVH93-G4S-CD16A-V158-FL-F-P2A- | 7626 | 8318 |
| IgHSP-CD20-VHH-USC1-2HCD26-GS4-KIR2DL1- | | |
| ECDTMCP-opt2-F-F2A-PAC | | |
| CD8SP-BCMA-FHVH93-G4S-CD16A-V158-FL-F-P2A- | 7627 | 8319 |
| IgHSP-CD20-VHH-USC1-2HCD26-GS4-OX40- | | |
| ECDTMCP-opt2-F-F2A-PAC | | |
| CD19-hu-mROO5-1-vL-huTCRg-d3-F-P2A-SP-CD19-hu- | 7628 | 8320 |
| mROO5-1-vH-huTCRd-d2-F-F2A-PAC | | |
| CD19-hu-mROO5-1-vL-huTCRg-d4-F-P2A-SP-CD19-hu- | 7629 | 8321 |
| mROO5-1-vH-huTCRd-d2-F-F2A-PAC | | |
| CD19-hu-mROO5-1-vL-huTCRg-d31-F-P2A-SP-CD19- | 7630 | 8322 |
| hu-mROO5-1-vH-huTCRd-d2-F-F2A-PAC | | |
| CD19-hu-mROO5-1-vL-huTCRg-d9-F-P2A-SP-CD19-hu- | 7631 | 8323 |
| mROO5-1-vH-huTCRd-d2-F-F2A-PAC | | |
| CD19-hu-mROO5-1-vL-huTCRg-d26-F-P2A-SP-CD19- | 7632 | 8324 |
| hu-mROO5-1-vH-huTCRd-d2-F-F2A-PAC | | |
| CD8SP-BCMA-FHVH93-G4S-CD16A-V158-FL-F-P2A- | 7633 | 8325 |
| IgHSP-CD20-VHH-USC1-2HCD26-GS4-CD32- | | |
| ECDTMCP-opt2-F-F2A-PAC | | |
| pCCLc-MNDU3-DEco-Nhe-CD19-hu-mROO5-1-vL- | 7634 | 8326 |
| huTCRg-d5-F-P2A-SP-CD19-hu-mROO5-1-vH-huTCRd- | | |
| d4-F-F2A-PAC | | |
| pCCLc-MNDU3-DEco-Nhe-CD19-hu-mROO5-1-vL- | 7635 | 8327 |
| huTCRg-F-P2A-SP-CD19-hu-mROO5-1-vH-huTCRd-F- | | |
| F2A-PAC | | |
| CD8SP-CD19-hu-mROO5-1-scFv-G3S-NKp46- | 7636 | 8328 |
| ECDTMCP-opt2-F-F2A-PAC | | |
| CD8SP-CD19-hu-mROO5-1-scFv-G3S-CD3e-ECDTMCP- | 7637 | 8329 |
| d9-opt2-F-F2A-PAC | | |
| CD8SP-CD19-hu-mROO5-1-scFv-G3S-CD3e-ECDTMCP- | 7638 | 8330 |
| d18-opt2-F-F2A-PAC | | |
| CD8SP-hCD19-EUK-5-13-vL-IgCL-NKp30-Hinge-TMCP- | 7639 | 8331 |
| opt1-F-P2A-SP-hCD19-EUK-5-13-vH-IgG1-CH1- | | |
| CD3zECDTMCP-opt2-F-F2A-PAC | | |
| CD8SP-NKp30-Ig-Hinge-opt1-[hTCRb-S57C]-F-P2A- | 7640 | 8332 |
| IgHSP-CD20-VHH-USC1-2HC2D6-G4S-CD19-hu- | | |
| mROO5-1-vH-[hTCRa-T48C-opt]-F-F2A-PAC | | |
| CD8SP-hCD19-EUK-5-13-vL-IgCL-NKp44-Hinge-TMCP- | 7641 | 8333 |
| opt1-F-P2A-SP-hCD19-EUK-5-13-vH-IgG1-CH1- | | |
| CD3zECDTMCP-opt2-F-F2A-PAC | | |
| CD8-hCD19-EUK-5-13-vL-IgCL-CD3zECDTMCP-opt-F- | 7642 | 8334 |
| P2A-SP-hCD19-EUK-5-13-vH-IgG1-CH1-CD16A-v158- | | |
| S197P-FL-v3-F-F2A-PAC | | |
| CD8SP-CD19-hu-mROO5-vL-IgCL-CD3zECDTMCP-opt- | 7643 | 8335 |
| F-P2A-SP-CD19-hu-mROO5-vH-IgGA1-CHI- | | |
| CD3zECDTMCP-opt2-F-F2A-PAC | | |
| CD8SP-CD19-hu-mROO5-vL-IgCL-CD3zECDTMCP-opt- | 7644 | 8336 |
| F-P2A-SP-CD19-hu-mROO5-vH-IgG2-1C-CHI- | | |
| CD3zECDTMCP-opt2-F-F2A-PAC | | |
| CD8SP-CD19-hu-mROO5-vL-IgCL-CD3zECDTMCP-opt- | 7645 | 8337 |
| F-P2A-SP-CD19-hu-mROO5-vH-IgD-CHI- | | |
| CD3zECDTMCP-opt2-F-F2A-PAC | | |
| CD8SP-CD19-hu-mROO5-vL-IgCL-CD3zECDTMCP-opt- | 7646 | 8338 |
| F-P2A-SP-CD19-hu-mROO5-vH-IgG2-0C- | | |
| CD3zECDTMCP-opt2-F-F2A-PAC | | |
| CD8SP-CD19-hu-mROO5-vL-IgCL-CD3zECDTMCP-opt- | 7647 | 8339 |
| F-P2A-SP-CD19-hu-mROO5-vH-IgGA2-CHI- | | |
| CD3zECDTMCP-opt2-F-F2A-PAC | | |
| CD8SP-CD19-hu-mROO5-vL-IgCL-CD3zECDTMCP-opt- | 7648 | 8340 |
| F-P2A-SP-CD19-hu-mROO5-vH-IgE-CHI- | | |
| CD3zECDTMCP-opt2-F-F2A-PAC | | |
| CD8SP-CD19-hu-mROO5-vL-IgCL-CD3zECDTMCP-opt- | 7649 | 8341 |
| F-P2A-SP-CD19-hu-mROO5-vH-IgG4-CHI- | | |
| CD3zECDTMCP-opt2-F-F2A-PAC | | |
| CD8SP-CD19-hu-mROO5-vL-IgCL-CD3zECDTMCP-opt- | 7650 | 8342 |
| F-P2A-SP-CD19-hu-mROO5-vH-IgG3-CHI- | | |
| CD3zECDTMCP-opt2-F-F2A-PAC | | |
| pLENTI-CD8-hCD19-EUK-5-13-vL-IgCL-CD8-hinge- | 7651 | 8343 |
| NKG2D-TM-2B4-CP-opt-1-CD3zCP-opt1-F-P2A-IgSP- | | |
| hCD19-EUK-5-13-vH-IgG1-CH1-DAP10-opt2-CD3zCP- | | |
| opt2-F-F2A-PAC | | |
| CD8SP-CD19-hu-mROO5-vL-IgCL-CD3zECDTMCP-opt- | 7652 | 8344 |
| F-P2A-SP-CD19-hu-mROO5-vH-IgM-CHI- | | |
| CD3zECDTMCP-opt2-F-F2A-PAC | | |
| CD19-hu-mROO5-1-vL-NKp30-ECDTMCP-opt1-F-P2A- | 7653 | 8345 |
| IgSP-CD19-hu-mROO5-1-vH-NKp30-ECDTMCP-opt2-F- | | |
| F2A-PAC | | |
| CD8SP-NKp30-Ig-Hinge-opt1-G4S-humROO5-vL- | 7654 | 8346 |
| [hTCRb-S57C]-F-P2A-IgHSP-NKp30-Ig-Hinge-opt2-G4S- | | |
| CD19-hu-mROO5-1-vH-[hTCRa-T48C-opt]-F-F2A-PAC | | |
| CD8SP-NKp44-Ig-opt1-[hTCRb-S57C]-F-P2A-IgHSP- | 7655 | 8347 |
| NKp30-Ig-Hinge-opt2-[hTCRa-T48C-opt]-F-F2A-PAC | | |
| CD8SP-BCMA-FHVH93-G4S-NKp44-Hinge-ECDTMCP- | 7656 | 8348 |
| opt1-F-P2A-IgHSP-CD20-VHH-USC1-2HCD26-G4S- | | |
| NKp44-Hinge-ECDTMCP-opt2-F2A-PAC | | |
| CD8SP-BCMA-FHVH93-G4S-NKp30-ECDTMCP-opt1- | 7657 | 8349 |
| F-P2A-IgHSP-CD20-VHH-USC1-2HC2D6-G4S-NKp30- | | |
| ECDTMCP-opt2-F-F2A-PAC | | |
| CD19-hu-mROO5-1-vL-NKp44-Hinge-TMCP-opt1-F- | 7658 | 8350 |
| P2A-IgSP-CD19-hu-mROO5-1-vH-NKp44-Hinge-TMCP- | | |
| opt2-F-F2A-PAC | | |
| CD19-hu-mROO5-1-vL-NKp44-ECDTMCP-opt1-F-P2A- | 7659 | 8351 |
| IgSP-CD19-hu-mROO5-1-vH-NKp44-ECDTMCP-opt2-F- | | |
| F2A-PAC | | |
| FMC64-CD16A-v158-S197P-FL-v3-F-F2A-PAC | 7660 | 8352 |
| CD20-2F2-CD16A-v158-S197P-FL-v3-F-F2A-PAC | 7661 | 8353 |
| CD20-Ubli-CD16A-v158-S197P-FL-v3-F-F2A-PAC | 7662 | 8354 |
| CD8SP-Hu161-2-CD16A-v158-S197P-FL-v3-F-F2A-PAC | 7663 | 8355 |
| BCMA-J6M0-CD16A-v158-S197P-FL-v3-F-F2A-PAC | 7664 | 8356 |
| CD8SP-SC22-HA22-CD16A-v158-S197P-FL-v3-F-F2A- | 7665 | 8357 |
| PAC | | |
| CD22-h10F4v2-CD16A-v158-S197P-FL-v3-F-F2A-PAC | 7666 | 8358 |
| CD8SP-hu-HA22-1-CD16A-v158-S197P-FL-v3-F-F2A- | 7667 | 8359 |
| PAC | | |
| hCD19-Bu12-CD16A-v158-S197P-FL-v3-F-F2A-PAC | 7668 | 8360 |
| CD8SP-CD20-VHH-USC1-2HC2D6-CD16A-v158-S197P- | 7669 | 8361 |
| FL-v3-F-F2A-PAC | | |
| CD8SP-CD38-331-vHH-D64E-CD16A-v158-S197P-FL- | 7670 | 8362 |
| v3-F-F2A-PAC | | |
| CD8SP-CD38-331-vHH-S53E-CD16A-v158-S197P-FL- | 7671 | 8363 |
| v3-F-F2A-PAC | | |
| CD8SP-BCMA917-vHH-E59D-CD16A-v158-S197P-FL- | 7672 | 8364 |
| v3-F-F2A-PAC | | |
| CD8SP-SARScov2-CR3022-CD16A-v158-S197P-FL-v3- | 7673 | 8365 |
| F-F2A-PAC | | |
| CD8SP-CD19-hu-mROO5-vL-IgCL-Hinge-CD16A-v158- | 7674 | 8366 |
| S197P-FL-v3-F-F2A-SP-CD19-hu-mROO5-vH-IgG1- | | |
| CH1-CD3zECDTMCP-opt2-F-F2A-PAC | | |
| CD8-hCD19-EUK-5-13-vL-IgCL-mutCD3z-ECDTM- | 7675 | 8367 |
| 2B4CP-opt1-CD3zCP-opt1-F-P2A-IgSP-hCD19-EUK-5- | | |
| 13-vH-IgG1-CH1-mutCD3z-ECDTM-2B4CP-opt2- | | |
| CD3zCP-opt2-F-F2A-PAC | | |
| CD8SP-CD19-hu-mROO5-1-CD16A-v158-S197P-FL-v3- | 7676 | 8368 |
| F-F2A-PAC | | |
| IgHSP-CD20-VHH-USC1-2HCD26-G4S-CD16A-v158- | 7677 | 8369 |
| FL-v2-F2A-PAC | | |
| CD8SP-BCMA-FHVH93-G4S-CD16A-V158-FL-F- | 7678 | 8370 |
| P2A::Xba-PAC | | |
| CD8SP-CD19-hu-mROO5-1-scFv-CD16A-v158-S197P- | 7679 | 8371 |
| FL-v3 | | |
| CD8SP-CD19-hu-mROO5-1-scFv-CD28-Ig-113-137- | 7680 | 8372 |
| NKp46-Hinge-TM-CP-opt2-F-F2A-PAC | | |
| CD8SP-CD19-hu-mROO5-1-scFv-CD28-Ig-113-137- | 7681 | 8373 |
| DNAM-1-Hinge-TMCP-opt2-F-F2A-PAC | | |
| CD8SP-CD19-hu-mROO5-1-scFv-CD28-Ig-113-137- | 7682 | 8374 |
| NKp46-Hinge-TM-CP-opt2-F-F2A-Synth-IL2m4-R38E- | | |
| F42A-SynthCD28TM | | |
| CD8SP-CD19-hu-mROO5-1-scFv-CD28-Ig-113-137- | 7683 | 8375 |
| CD16A-v158-Hinge-TM-CP-v2-F-F2A-PAC | | |
| CD8SP-CD19-hu-mROO5-1-scFv-CD28-Ig-113-137- | 7684 | 8376 |
| CD3eECDTMCP-opt2-F-F2A-PAC | | |
| CD8SP-CD19-hu-mROO5-1-scFv-CD28-Ig-113-137- | 7685 | 8377 |
| NKp44-Hinge-TMCP-opt2-F-F2A-Synth-IL2m4-R38E- | | |
| F42A-SynthCD28TM | | |
| NKG2D-opt2-G4Sx3-BCMA917-vHH-F-F2A-Synth- | 7686 | 8378 |
| IL2m4-R38E-F42A-SynthCD28TM | | |
| NKG2D-opt2-G4Sx3-BCMA917-vHH-F-F2A-Synth-IL2- | 7687 | 8379 |
| tBCMA-L244 | | |
| CD8SP-CD19-hu-mROO5-1-scFv-G3S-NKp46- | 7688 | 8380 |
| ECDTMCP-opt2-F-F2A-PAC | | |
| CD8SP-CD19-hu-mROO5-1-scFv-G3S-CD3e-ECDTMCP- | 7689 | 8381 |
| d9-opt2-F-F2A-PAC | | |
| CD8SP-CD19-hu-mROO5-1-scFv-G3S-CD3e-ECDTMCP- | 7690 | 8382 |
| d18-opt2-F-F2A-PAC | | |
| CD8SP-CD19-hu-mROO5-1-scFv-CD8-hinge-DNAM-1- | 7691 | 8383 |
| Hinge-TMCP-opt2-F-F2A-PAC | | |
| CD8SP-CD19-hu-mROO5-1-scFv-CD8-hinge-NKp44- | 7692 | 8384 |
| Hinge-TMCP-opt2-F-F2A-PAC | | |
| CD8SP-CD19-hu-mROO5-1-scFv-CD8-hinge-CD16A- | 7693 | 8385 |
| Hinge-TM-CP-V158-F-P2A-PAC | | |
| CD8SP-CD19-hu-mROO5-1-scFv-CD8-hinge-DAP10- | 7694 | 8386 |
| opt2-CD3zCP-opt2-F-F2A-PAC | | |
| CD8SP-CD19-hu-mROO5-1-scFv-CD8-hinge-NKp46- | 7695 | 8387 |
| Hinge-TM-CP-opt2-F-F2A-PAC | | |
| NKG2D-opt2-G4Sx3-Her2-47D5-vHH-F-F2A-PAC | 7696 | 8388 |
| NKG2D-opt2-G4Sx3-Her3-21F06-vHH-F-F2A-PAC | 7697 | 8389 |
| NKG2D-opt2-G4Sx3-BCMA917-vHH-F-F2A-PAC | 7698 | 8390 |
| Nkp80-G4Sx3-opt1-Her3-21F06-vHH-F-F2A-PAC | 7699 | 8391 |
| CD8SP-CD19-hu-mROO5-1-scFv-NKp44-ECDTMCP- | 7700 | 8392 |
| opt2-F-F2A-hIL2-CD28TM | | |
| CD8-hCD19-EUK-5-13-vL-IgCL-CD3zECDTMCP-opt-F- | 7701 | 8393 |
| P2A-SP-hCD19-EUK-5-13-vH-IgG1-CHI- | | |
| CD3zECDTMCP-opt2-F-F2A-hIL2-CD28TMter | | |
| CD8SP-CD19-hu-mROO5-1-scFv-G3S-NKp46- | 7702 | 8394 |
| ECDTMCP-opt2-F-F2A-PAC | | |
| TCRgd-G5-4-G2SG-Synth-IL2m10-1-R38A-F42K- | 7703 | 8395 |
| Q126A-U60 | | |
| TCRgd-G5-4-G2SG-Synth-IL2m4-1-R38E-F42A-I92A- | 7704 | 8396 |
| U60 | | |
| TCRgd-G5-4-G2SG-Synth-IL2v-Roche-F42A-Y45A- | 7705 | 8397 |
| L72G-C125A-U60 | | |
| TCRgd-G5-4-G2SG-Synth-hIL2-U60 | 7706 | 8398 |
| CD8-hCD19-EUK-5-13-vL-IgCL-CD3zECDTMCP-opt-F- | 7707 | 8399 |
| P2A-SP-hCD19-EUK-5-13-vH-IgG1-CH1- | | |
| CD3zECDTMCP-opt2-F-F2A-hIL2-CD28TMter | | |
| CD8SP-MSLN-7D9-HL-scFv-Acc65I-G4S-humROO5-1- | 7708 | 8400 |
| vL-[hTCRb-KACIAH]-F-P2A-IgHSP-hu-PSMA-J591- | | |
| scFv-G4S-CD19-hu-mROO5-1-vH-[hTCRa-CSDVP]-F- | | |
| F2A-PAC | | |
| CD19-hu-mROO5-1-vL-huTCRg2-F-P2A-SP-CD19-hu- | 7709 | 8401 |
| mROO5-1-vH-huTCRd-d2-F-F2A-PAC | | |
| CD8SP-CD19-hu-mROO5-1-scFv-NKp30-ECDTMCP- | 7710 | 8402 |
| opt2-F-F2A-Synth-IL2m4-R38E-F42A-SythCD28TM | | |
| CD8SP-CD19-hu-mROO5-1-scFv-NKp30-ECDTMCP- | 7711 | 8403 |
| opt2-F-F2A-Synth-IL2m10-1-R38A-F42K-Q126A-Syth- | | |
| CD28TM | | |
| CD8SP-CD19-hu-mROO5-1-scFv-NKp30-ECDTMCP- | 7712 | 8404 |
| opt2-F-F2A-Synth-IL2v-Roche-T3A-F42A-Y45A-L72G- | | |
| C125A-SythCD28TM | | |
| CD8SP-CD19-hu-mROO5-1-scFv-NKp30-ECDTMCP- | 7713 | 8405 |
| opt2-F-F2A-Synth-IL2m4-1-R38E-F42A-I92A- | | |
| SythCD28TM | | |
| CD8SP-CD19-hu-mROO5-1-scFv-NKp30-ECDTMCP- | 7714 | 8406 |
| opt2-F-F2A-Synth-hIL15-E64K-SythCD28TM | | |
| CD8SP-CD19-hu-mROO5-1-scFv-NKp30-ECDTMCP- | 7715 | 8407 |
| opt2-F-F2A-Synth-hIL15-L69R-SythCD28TM | | |
| CD8SP-CD19-hu-mROO5-1-scFv-NKp30-ECDTMCP- | 7716 | 8408 |
| opt2-F-F2A-Synth-hIL15-D8S-SythCD28TM | | |
| CD19-hu-mROO5-1-vL-huTCRg-d5-F-P2A-SP-CD19-hu- | 7717 | 8409 |
| mROO5-1-vH-huTCRd-d2-F-F2A-Synth-IL2m4-A11D- | | |
| R38E-F42A-SythCD28TM | | |
| CD19-hu-mROO5-1-vL-huTCRg-d5-F-P2A-SP-CD19-hu- | 7718 | 8410 |
| mROO5-1-vH-huTCRd-d2-F-F2A-Synth-IL2m10-1-R38A- | | |
| F42K-Q126A-Syth-CD28TM | | |
| CD19-hu-mROO5-1-vL-huTCRg-d5-F-P2A-SP-CD19-hu- | 7719 | 8411 |
| mROO5-1-vH-huTCRd-d2-F-F2A-Synth-IL2v-Roche- | | |
| T3A-F42A-Y45A-L72G-C125A-SythCD28TM | | |
| CD19-hu-mROO5-1-vL-huTCRg-d5-F-P2A-SP-CD19-hu- | 7720 | 8412 |
| mROO5-1-vH-huTCRd-d2-F-F2A-Synth-IL2m4-1-R38E- | | |
| F42A-I92A-SythCD28TM | | |
| CD19-hu-mROO5-1-vL-huTCRg-d5-F-P2A-SP-CD19-hu- | 7721 | 8413 |
| mROO5-1-vH-huTCRd-d2-F-F2A-Synth-hIL15-E64K- | | |
| SythCD28TM | | |
| CD19-hu-mROO5-1-vL-huTCRg-d5-F-P2A-SP-CD19-hu- | 7722 | 8414 |
| mROO5-1-vH-huTCRd-d2-F-F2A-Synth-hIL15-D8S- | | |
| SythCD28TM | | |
| CD19-hu-mROO5-1-vL-huTCRg-d5-F-P2A-SP-CD19-hu- | 7723 | 8415 |
| mROO5-1-vH-huTCRd-d2-F-F2A-Synth-hIL15-L69R- | | |
| SythCD28TM | | |
| CD8SP-CD19-hu-mROO5-1-G4Sx3-CD16A-v158-S197P- | 7724 | 8416 |
| FL-v3-F-F2A-Synth-IL2m10-1-R38A-F42K-Q126A-Syth- | | |
| CD28TM | | |
| CD8SP-CD19-hu-mROO5-1-G4Sx3-CD16A-v158-S197P- | 7725 | 8417 |
| FL-v3-F-F2A-Synth-IL2v-Roche-T3A-F42A-Y45A-L72G- | | |
| C125A-SythCD28TM | | |
| CD8SP-CD19-hu-mROO5-1-G4Sx3-CD16A-v158-S197P- | 7726 | 8418 |
| FL-v3-F-F2A-Synth-IL2m4-1-R38E-F42A-I92A- | | |
| SythCD28TM | | |
| CD8SP-CD19-hu-mROO5-1-G4Sx3-CD16A-v158-S197P- | 7727 | 8419 |
| FL-v3-F-F2A-Synth-hIL15-L69R-SythCD28TM | | |
| CD8SP-CD19-hu-mROO5-1-G4Sx3-CD16A-v158-S197P- | 7728 | 8420 |
| FL-v3-F-F2A-Synth-IL2m4-R38E-F42A-SythCD28TM | | |
| CD8SP-CD19-hu-mROO5-1-G4Sx3-CD16A-v158-S197P- | 7729 | 8421 |
| FL-v3-F-F2A-Synth-hIL15-E64K-SythCD28TM | | |
| CD8SP-CD19-hu-mROO5-1-G4Sx3-CD16A-v158-S197P- | 7730 | 8422 |
| FL-v3-F-F2A-Synth-hIL15-D8S-SythCD28TM | | |
| CD19-hu-mROO5-1-vL-huTCRg-d5-F-P2A-SP-CD19-hu- | 7731 | 8423 |
| mROO5-1-vH-huTCRd-d2-F-F2A-Synth-IL2m4-R38E- | | |
| F42A-SythCD28TM | | |
| CD8SP-CD19-hu-mROO5-1-vL-TCRbECD- | 7732 | 8424 |
| CD3zECDTMCP-opt-F-P2A-SP-CD19-hu-mROO5-1-vH- | | |
| hTCRaECD-CD3zECDTMCP-opt2-F-F2A-Synth-IL2m4- | | |
| R38E-F42A-SythCD28TM | | |
| CD8SP-CD19-hu-mROO5-1-vL-TCRbECD- | 7733 | 8425 |
| CD3zECDTMCP-opt-F-P2A-SP-CD19-hu-mROO5-1-vH- | | |
| hTCRaECD-CD3zECDTMCP-opt2-F-F2A-Synth-IL2m10- | | |
| 1-R38A-F42K-Q126A-Syth-CD28TM | | |
| CD8SP-CD19-hu-mROO5-1-vL-TCRbECD- | 7734 | 8426 |
| CD3zECDTMCP-opt-F-P2A-SP-CD19-hu-mROO5-1-vH- | | |
| hTCRaECD-CD3zECDTMCP-opt2-F-F2A-Synth-hIL15- | | |
| D8S-SythCD28TM | | |
| CD8SP-CD19-hu-mROO5-1-vL-TCRbECD- | 7735 | 8427 |
| CD3zECDTMCP-opt-F-P2A-SP-CD19-hu-mROO5-1-vH- | | |
| hTCRaECD-CD3zECDTMCP-opt2-F-F2A-Synth-hIL15- | | |
| L69R-SythCD28TM | | |
| CD8SP-CD19-hu-mROO5-1-vL-TCRbECD- | 7736 | 8428 |
| CD3zECDTMCP-opt-F-P2A-SP-CD19-hu-mROO5-1-vH- | | |
| hTCRaECD-CD3zECDTMCP-opt2-F-F2A-Synth-hIL2- | | |
| CD28TM | | |
| CD8SP-CD19-hu-mROO5-1-vL-TCRbECD- | 7737 | 8429 |
| CD3zECDTMCP-opt-F-P2A-SP-CD19-hu-mROO5-1-vH- | | |
| hTCRaECD-CD3zECDTMCP-opt2-F-F2A-IgH-SP1- | | |
| tBCMA-L244 | | |
| CD8SP-CD19-hu-mROO5-1-vL-TCRbECD- | 7738 | 8430 |
| CD3zECDTMCP-opt-F-P2A-SP-CD19-hu-mROO5-1-vH- | | |
| hTCRaECD-CD3zECDTMCP-opt2-F-F2A-Synth-IL2- | | |
| tBCMA-L244 | | |
| CD8SP-CD19-hu-mROO5-1-CD28-Ig-113-137-NKp44- | 7739 | 8431 |
| Hinge-TMCP-opt2-F-F2A-Synth-IL2-tBCMA-L244 | | |
| CD8SP-CD19-hu-mROO5-1-CD28-Ig-113-137-NKp46- | 7740 | 8432 |
| Hinge-TM-CP-opt2-F-F2A-Synth-IL2-tBCMA-L244 | | |
| NKG2D-opt2-G4Sx3-BCMA917-vHH-F-F2A-Synth- | 7741 | 8433 |
| IL2m4-R38E-F42A-SynthCD28TM | | |
| NKG2D-opt1-G4Sx3-hu-mROO5-1-vL-F-P2A-NKG2D- | 7742 | 8434 |
| opt2-G4Sx3-CD19-hu-mROO5-1-vH-F-F2A-PAC | | |
| G4Sx3v2-hu-mROO5-1-vL-F-P2A-CD94-G4Sx3-CD19- | 7743 | 8435 |
| hu-mROO5-1-vH-F-F2A-PAC | | |
| NKG2H-opt1-G4Sx3v2-hu-mROO5-1-vL-F-P2A-CD94- | 7744 | 8436 |
| G4Sx3-CD19-hu-mROO5-1-vH-F-F2A-PAC | | |
| NKG2A-opt1-G4Sx3v2-hu-mROO5-1-vL-F-P2A-CD94- | 7745 | 8437 |
| G4Sx3-CD19-hu-mROO5-1-vH-F-F2A-PAC | | |
| CD8SP-hu-Hsp70-USC1-hCD8TM-BBZ-XS-T2A-PAC | 7746 | 8438 |
| CD8SP-cmHsp701-hCD8TM-BBZ-XS-T2A-PAC | 7747 | 8439 |

TABLE 49
Exemplary diseases targeted by SARs.

| SAR “X” | EXEMPLARY DISEASE TARGETED BY SARs (e.g., CD16 SAR, |
|---|---|
| TARGET | NKp30 SAR, NKp44 SAR, NKp46 SAR and DAP10 SAR etc.) |
| CD19 | ALL, CLL, lymphoma, lymphoid blast crisis of CML, multiple |
| | myeloma, immune disorders |
| ALK | Non-small Cell Lung Cancer (NSCLC), ALCL (anaplastic large cell |
| | lymphoma), IMT (inflammatory myofibroblastic tumor), or |
| | neuroblastoma |
| CD45 | Blood cancers |
| BCMA | Myeloma, PEL, plasma cell leukemia, Waldenstrom's |
| | macroglobinemia |
| CD5 | Blood cancer, T cell leukemia, T cell lymphoma |
| CD20 | Blood cancers, Leukemia, ALL, CLL, lymphoma, immune disorders |
| CD22 | Blood cancers, Leukemia, ALL, CLL, lymphoma, lymphoid blast crisis |
| | of CML, immune disorders |
| CD23 | Blood cancers, Leukemia, ALL, CLL, lymphoma, autoimmune |
| | disorders |
| CD30 | Hodgkins's lymphoma, Cutaneous T cell lymphoma |
| CD32 | Solid tumors |
| CD33 | Blood cancers, AML, MDS |
| CD34 | Blood cancers, AML, MDS |
| CD44v6 | Blood cancers, AML, MDS |
| CD70 | Blood cancers, lymphoma, myeloma, Waldenstrom's |
| | macroglobulinemia, Kidney cancer |
| CD79b | Blood cancers, ALL, Lymphoma |
| CD123 | Blood cancers, AML, MDS |
| CD138 | Blood cancers, Myeloma, PEL, plasma cell leukemia, waldenstrom's |
| | macroglobulinemia |
| CD179b | Blood cancers, ALL, Lymphoma |
| CD276/B7-H3 | Ewing's sarcoma, neuroblastoma, rhabdomyosarcoma, ovarian, |
| | colorectal and lung cancers |
| CD324 | Solid tumors, esophageal, prostate, colorectal, breast, lung cancers |
| CDH6 | Solid tumors, renal, ovarian, thyroid cancers |
| CDH17 | Adenocarciniomas, gastrointestinal, lung, ovarian, endometrial cancers |
| CDH19 | Solid tumor, Melanoma |
| EGFR | Colon cancer, lung cancer |
| CLEC5A | Blood cancers, Leukemia, AML |
| GR/LHR | Prostate cancer, ovarian cancer or breast cancer |
| CLL1 | Blood cancer, Leukemia |
| CMVpp65 | CMV infection, CMV colitis, CMV pneumonitis |
| CS1 | Blood cancers, myeloma, PEL, plasma cell leukemia |
| CSF2RA | AML, CML, MDS |
| CD123 | Blood cancers, AML, MDS |
| DLL3 | Melanoma, lung cancer or ovarian cancer |
| EBNA3c/MHC I | Epstein Barr virus infection and related diseases including cancers |
| EBV-gp350 | Epstein Barr virus infection and related diseases |
| EGFR | Solid tumors, Colon cancer, lung cancer |
| EGFRvIII | Solid tumors, glioblastoma |
| EpCam1 | Gastrointestinal cancer |
| FLT3 | Blood cancers, AML, MDS, ALL |
| Folate Receptor | Ovarian cancer, NSCLC, endometrial cancer, renal cancer, or other |
| alpha(FR1 or | solid tumors |
| FOLR1) | |
| FSHR | Prostate cancer, ovarian cancer or breast cancer |
| GD2 | Neuroblastoma |
| GD3 | Melanoma |
| GFRa4 | Cancer, thyroid medullary cancer |
| Fucosyl- | Small cell lung cancer |
| GM1(GM1) | |
| GPRC5D | Myeloma, PEL, plasma cell leukemia, waldenstrom's |
| | macroglobulinemia |
| gp100 | Melanoma |
| GPC3 | Solid tumors, Lung cancer |
| gpNMB | Melanoma, brain tumors, gastric cancers |
| GRP78 | Myeloma |
| Her2 | Solid tumors, breast cancer, stomach cancer |
| Her3 | Colorectal, breast cancer |
| HMW-MAA | Melanoma |
| HTLV1- | HTLV1 infection associated diseases, Adult T cell leukemia-lymphoma |
| TAX/MHC I | |
| IL11Ra | Blood cancers, AML, ALL, CML, MDS, sarcomas |
| IL6Ra | Solid tumors, Liver cancer |
| IL13Ra2 | Glioblastomas |
| KSHV-K8.1 | Kaposi's sarcoma, PEL, Multicentric Castleman's disease |
| LAMP1 | Blood cancers, AML, ALL, MDS, CLL, CML |
| LewisY | Cancers |
| L1CAM | Solid tumors, ovarian, breast, endometrial cancers, melanoma |
| LHR | Prostate cancer, ovarian cancer or breast cancer |
| Lym1 | Blood cancer, Leukemia, Lymphoma |
| Lym2 | Blood cancer, Leukemia, Lymphoma |
| CD79b | Blood cancers, lymphoma |
| MART1/MHC I | Melanoma |
| Mesothelin | Mesothelioma, ovarian cancer, pancreatic cancer |
| Muc1/MHC I | Breast cancer, gastric cancer, colorectal cancer, lung cancer, or other |
| | solid tumors |
| Muc16 | Ovarian cancer |
| NKG2D | Leukemia, lymphoma or myeloma |
| NYBR1 | Breast cancer |
| PSCA | Prostate cancer |
| PR1/MHC I | Blood cancer, Leukemia |
| Prolactin | Breast cancer, chromophobe renal cell cancer |
| Receptor | |
| PSMA | Prostate cancer |
| PTK7 | Melanoma, lung cancer or ovarian cancer |
| ROR1 | Blood cancer, B cell malignancy, lymphoma, CLL |
| SLea | Pancreatic cancer, colon cancer |
| SSEA4 | Pancreatic cancer |
| Tyrosinase/MHC | Melanoma |
| I | |
| TCRB1 | T cell leukemias and lymphomas, autoimmune disorders |
| TCRB2 | T cell leukemias and lymphomas, autoimmune disorders |
| TCRgd | T cell leukemias and lymphomas, autoimmune disorders |
| hTERT | Solid tumors, blood cancers |
| TGFBR2 | Solid tumors, keloid |
| TIM1/HAVCR1 | Kidney cancer, liver cancer |
| TROP2 | Solid tumors, Breast cancer, prostate cancer |
| TSHR | Thyroid cancer, T cell leukemia, T cell Lymphoma |
| TSLPR | Blood cancers, Leukemias, AML, MDS |
| Tyrosinase/MHC | Melanoma |
| I | |
| VEGFR3 | Solid tumors |
| WT1/MHC I | Blood cancers, AML |
| Folate Receptorβ | AML, Myeloma |
| B7H4 | Breast cancer or ovarian cancer |
| CD23 | Blood cancers, Leukemias, CLL |
| GCC | Gastrointestinal cancer |
| CD200R | Blood cancers, AML, MDS |
| AFP/MHC I | Solid tumors, Liver cancer |
| CD99 | Liver cancer |
| GPRC5D | Myeloma, waldenstrom's macroglobinemia |
| HPV16- | HPV16 associated cancers, cervical cancer, head and neck cancers |
| E7/MHC I | |
| Tissue Factor 1 | Solid tumors |
| (TF1) | |
| Tn-Muc1 | Solid tumors and blood cancers |
| Igk-Light Chain | Myeloma, plasma cell leukemia |
| Ras G12V/ | Solid tumors and blood cancers |
| MHC I | |
| CLD18A2 | Gastric, pancreatic, esophageal, ovarian, or lung cancer |
| (Claudin 18.2) | |
| CD43 | Blood cancers, AML |
| NY-ESO- | Myeloma |
| 1/MHC I | |
| MPL/TPO-R | Blood cancer, AML, MDS, CML, ALL, Myeloproliferative disorders, |
| | Polycythemia vera, Myelofibrosis, Essential Polycythemia |
| P-glycoprotein | Renal cancer, liver cancer, Myeloma |
| (MDR1) | |
| CD179a | Blood cancers, Acute Leukemia, CLL, ALL, Lymphoma |
| STEAP1 | Gastric or prostate cancer, or lymphoma |
| Livl (SLC39A6) | Breast or prostate cancer |
| Nectin4 | Bladder, renal, cervical, lung, head and neck or breast cancer |
| (PVRL4) | |
| Cripto (TDGF1) | Colorectal or endometrial or ovarian cancer |
| gpA33 | Colorectal or endometrial or ovarian cancer |
| FLT3 | Blood cancers, AML, ALL, MDS |
| BST1/CD157 | Blood cancers, AML, MDS |
| IL1RAP | Liver, colorectal, cervical, lung or ovarian cancer |
| Chloride channel | Glioma |
| IgE | Allergy |
| HLA-A2 | Graft vs host disease, tissue rejection (SIR Expressed in regulatory T |
| | cells) |
| Amyloid | Amyloidoses, alzheimer's disease |
| HIV1-env | HIVI/AIDS and related conditions |
| HIV1-gag | HIV1/AIDS and related conditions |
| Influenza A HA | Influenza A infection |

In one embodiment, the disclosure provides a SAR (such as an isolated SAR) comprising one or more heterologous antigen binding domains that are operationally linked via optional linkers to a naturally occurring (i.e., native or endogenous) receptor or a variant thereof. In an embodiment, the SAR retains the binding capability and functions of the native receptor but also acquires the binding capabilities and functions conferred by the one or more heterologous antigen binding domains. In an embodiment, the SAR retains partially or completely the binding capabilities and functions of the native receptor. In an embodiment, the SAR acquires the binding capabilities and functions conferred by the one or more heterologous antigen binding domains.

In one embodiment, the naturally occurring receptor is any receptor expressed on the surface of a cell (e.g., an immune cell, e.g., an immune effector cells). In an exemplary embodiment, the immune cell is selected from but not limited to a T cell, an NK cell, a monocyte/macrophage, a granulocyte and a B cell. In an embodiment, the naturally occurring signaling receptor is expressed on the surface of an immune cell (e.g., T cell, NK cell, NKT cell, macrophage, dendritic cell etc.).

In an embodiment, a naturally occurring receptor induces cell signaling, i.e., it is a naturally occurring signaling receptor. The naturally occurring signaling receptor may be an activating receptor (i.e., it induces cellular activation) or an inhibitory receptor (i.e., it blocks cell activation). In an embodiment, the naturally occurring receptor is an NK cell receptor, e.g., an NK activating or an NK inhibitory receptor. In an embodiment, the naturally occurring signaling receptor may be a receptor that induces cytotoxicity. In another embodiment, the naturally occurring signaling receptor may be a receptor that provides co-stimulation.

In an embodiment, the naturally occurring receptor that can be used in the construction of a SAR of the disclosure possesses a transmembrane domain. In an embodiment, a naturally occurring receptor is capable of recruiting a transmembrane adaptor protein. In an embodiment, a naturally occurring receptor is capable of recruiting a transmembrane adaptor protein selected from the group of but not limited to CD3ƒ, FcRy, DAP10, DAP12 or variants or fragments thereof. Exemplary such receptors include CD16A, NKp30, NKp44, NKp46 and NKG2D. In an embodiment, a naturally occurring receptor is capable of recruiting a transmembrane adaptor protein comprising a negatively charged residue (e.g., aspartate) within its transmembrane region. In an embodiment, a naturally occurring receptor possesses a transmembrane domain comprising a positively charged residue (lysine or arginine) that interacts with a negatively charged residue (e.g., aspartate) within the transmembrane region of a signaling adaptor protein. In an embodiment, the naturally occurring receptor possesses a transmembrane domain and a cytosolic domain. In an embodiment, the naturally occurring receptor possesses a hinge (spacer) domain and a transmembrane domain. In an embodiment, the naturally occurring receptor possesses a hinge (space) domain, a transmembrane domain and a cytosolic domain. An exemplary such receptor is CD16A. In an embodiment, the naturally occurring receptor possesses a hinge (space) domain, a membrane anchoring domain (e.g., GPI linked domain) but lacks a cytosolic domain. An exemplary such receptor is CD16B.

In exemplary embodiments, naturally occurring receptors that can be used in the construction of SAR of the disclosure include but are not limited to CD16A, CD16B, CD64, CD32, NKp30, NKp44, NKp46, KIR2DL1, KIR2DL2, KIR2DL3, KIR2DL5A, KIR2DL5B, KIR3DL1, KIR3DL2, KIR3DL4, KIR2DL4, KIR2DS1, KIR2DS2, KIR2DS3, KIR2DS4, KIR2DS5, KIR3DS1, NKG2D, NKG2C, NKG2A, NKG2E, NKG2F, DNAM-1, 2B4, OX40, CD28, 4-1BB, CD27, CD81, CD2, CD5, TNFR-I, TNFR-II, Fas, CD30, CD40, CRTAM, TIGIT, CD96, SLAMF6, SLAMF7, CD100, CD160, CEACAM, ILT2, KLRG1, LAIR1, CD161, Siglec3, Siglec-7, Siglec-9, TCRαβ and TCRγδ etc. and variants and fragments thereof.

In an embodiment, a naturally occurring receptor that can be used in the construction of a SAR of the disclosure is not a T cell receptor. In an embodiment, a naturally occurring receptor that can be used in the construction of a SAR of the disclosure is not TCRα, TCRβ, TCRγ, TCRδ or pre-TCRα. In an embodiment, a SAR does not comprise the constant chain of TCRα, TCRβ, TCRγ, TCRδ or pre-TCRα. In an embodiment, a SAR does not comprise the transmembrane domain of TCRα, TCRβ, TCRγ, TCRδ or pre-TCRα. In an embodiment, a SAR does not comprise the entire extracellular domain of TCRα, TCRβ, TCRγ, TCRδ or pre-TCRα. In an embodiment, the naturally occurring non-T cell receptor that can be used in the construction of a SAR of the disclosure comprises an intracellular activation domain. In an embodiment, the activation domain comprises one or more ITAMs. In an embodiment, the activation domain comprises one or more ITIMs. In an embodiment, the naturally occurring non-T cell receptor that can be used in the construction of a SAR of the disclosure comprises a costimulatory domain.

In exemplary embodiments, a SAR comprises intracellular activation domains derived from CD3ƒ, FcRγ, DAP10 or DAP12.

In an embodiment, the disclosure provides SAR comprising one or more heterologous antigen binding domains that are operationally linked to the entire extracellular domain of a non-TCR naturally occurring receptor, i.e., the receptor that is not TCRα, TCRβ, TCRγ, TCRδ or pre-TCRα. In an embodiment, the disclosure provides SAR comprising one or more heterologous antigen binding domains that are operationally linked to the partial extracellular domain of a non-TCR naturally occurring receptor. In an embodiment, the disclosure provides SAR comprising one or more heterologous antigen binding domains that are operationally linked to the partial extracellular domain of a non-TCR naturally occurring receptor. In an embodiment, the disclosure provides SAR comprising one or more heterologous antigen binding domains that are operationally linked to the entire or partial extracellular domain of a NTCRM (non-T cell receptor module). In an embodiment, the naturally occurring signaling receptor that can be used in the construction of a SAR of the disclosure is not CD4, CD8, CD28, CD27, CD16A or NKG2D.

In an embodiment, the disclosure provides SAR comprising one or more heterologous antigen binding domains that are operationally linked to the entire or partial extracellular domain of a naturally occurring receptor via optional linkers wherein the receptor is not part of TCR/CD3 receptor complex. In an embodiment, the disclosure provides SAR comprising one or more heterologous antigen binding domains that are operationally linked to the entire or partial extracellular domain of a naturally occurring receptor polypeptide chain via optional linkers wherein the receptor polypeptide chain is not part of TCR/CD3 receptor complex. In an embodiment, the disclosure provides SAR comprising one or more heterologous antigen binding domains that are operationally linked via optional linkers to the entire or partial extracellular domain of a naturally occurring receptor or a variant or a fragment thereof wherein the receptor does not associate with the TCR/CD3 receptor complex.

In an embodiment, the disclosure provides SAR comprising one or more heterologous antigen binding domains that are operationally linked to the entire or partial antigen (or ligand)-binding domain of a naturally occurring receptor or a variant or fragment thereof.

In an embodiment, the disclosure provides a SAR comprising one or more heterologous antigen binding domains that are operationally linked to the entire or partial antigen (or ligand)-binding domain of a non-TCR naturally occurring signaling receptor or an NTCRM or a variant or fragment thereof. In an embodiment, the disclosure provides a SAR comprising one or more heterologous antigen binding domains that are operationally linked to the entire or partial antigen (or ligand)-binding domain of a non-TCR signaling receptor or an NTCRM or a variant or a fragment thereof. In an embodiment, the disclosure provides a SAR comprising one or more heterologous antigen binding domains that are operationally linked to the entire or partial antigen (or ligand)-binding domain of a naturally occurring signaling receptor or a variant or fragment thereof wherein the naturally occurring signaling receptor is not TCRα, TCRβ, TCRγ, TCRδ or preTCRα. In an embodiment, the SAR of the disclosure retains at least some of the antigen binding properties of the non-TCR naturally occurring signaling receptor. In an embodiment, a SAR of the disclosure acquires novel antigen binding properties conferred by one or more of the heterologous antigen binding domains. In an embodiment, a SAR of the disclosure retains at least some of the antigen binding properties of the non-TCR naturally occurring signaling receptor and acquires novel antigen binding properties conferred by one or more of the heterologous antigen binding domains.

In an embodiment, the disclosure provides a double chain SAR comprising heterologous antigen binding domains derived from variable domains of a TCR (i.e., Vα, Vβ, Vδ or Vγ) that are operationally linked via optional linkers to the entire or partial extracellular domain of a naturally occurring signaling receptor. In an embodiment, the naturally occurring signaling receptor is an NTCRM (non-T cell receptor module).

In an embodiment, the disclosure provides a double chain SAR comprising heterologous antigen binding domains derived from variable domains of a TCR (i.e., Vα, Vβ, Vδ or Vγ) that are operationally linked via optional linkers to the hinge domain of an NTCRM (non-T cell receptor module) and/or a signaling adaptor (e.g., CD3ƒ, FcRγ, DAP10, DAP10 etc.) or variants or fragments thereof.

In an embodiment, the SAR does not comprise the entire extracellular domain of TCRα, TCRβ, TCRγ, TCRδ or pre-TCRα. In an embodiment, the SAR does not comprise the transmembrane domain of TCRα, TCRβ, TCRγ, TCRδ or pre-TCRα.

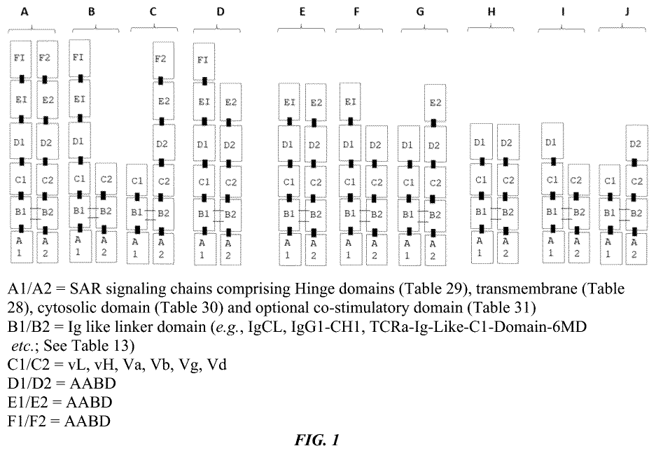

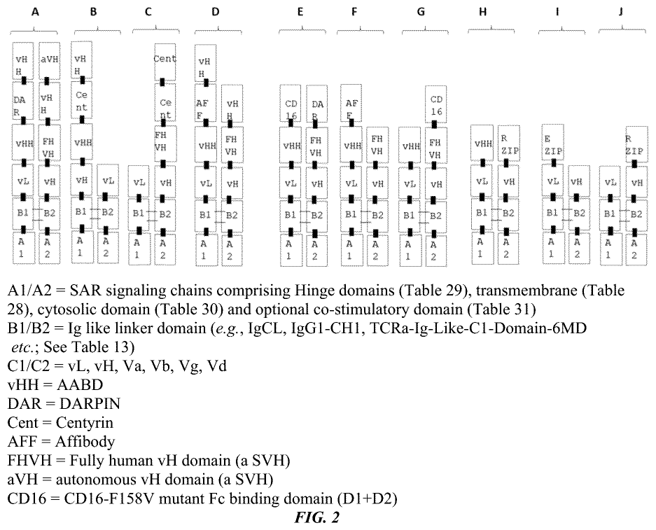

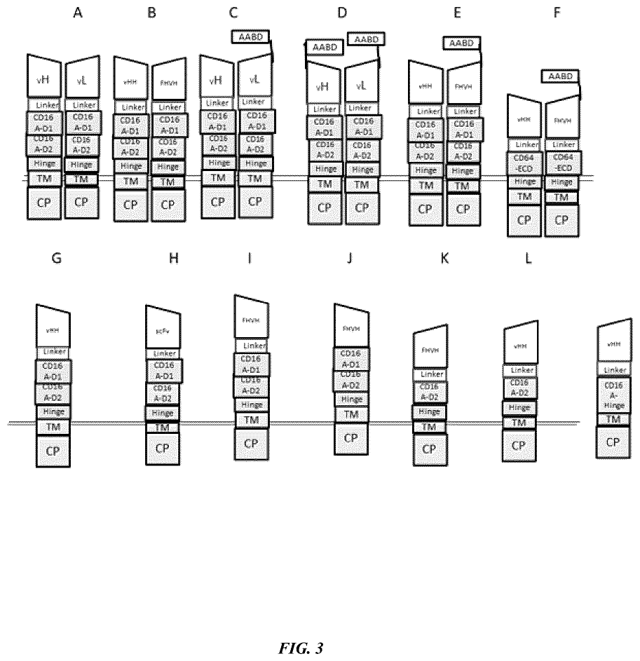

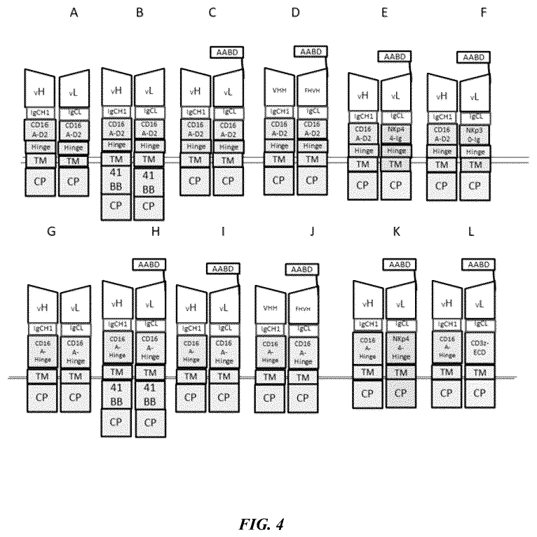

The schematic representations of the SARs of the disclosure are provided in FIGS. 1-5 and Tables A1-1 to A1-19.

In an embodiment, a naturally occurring receptor that can be used in the construction of a SAR of the disclosure may comprise a single polypeptide chain or multiple polypeptide chains. A naturally occurring receptor may be a component of a multi-chain receptor complex (e.g., T cell receptor complex).

In an embodiment, the SAR comprises more than one antigen binding domain. In an embodiment, the SAR comprise one or more heterologous (or non-naturally occurring) antigen binding domains. The exemplary heterologous antigen binding domains that can be used in the construction of the SAR of the disclosure include an autonomous antigen binding domain (e.g., fully human vH domain, vHH domain, single chain TCR, recombinant TCR or svd-TCR etc.), scFv, antibody, antibody fragment (vL, vH, Fab etc.), non-immunoglobulin antigen binding domain (e.g., Centyrin, affibody, DARPIN, ZIP domain, an adaptor, etc.), ligand, extracellular domain of a receptor (e.g., CD16A extracellular domain, NKp30 extracellular domain etc.), an autoantigen, a TCR, a TCR variable fragment (e.g., Va, Vb, Vg, Vd etc.) and variants and fragments thereof etc. In an exemplary embodiment, the one or more heterologous antigen binding domains comprise scTCR, svd-TCR or a TCR mimic scFv or a fragment thereof. In some embodiments, the SAR acquires TCR-like binding capabilities, e.g., ability to bind to a peptide/MHC complex.

The second-generation chimeric antigen receptor constructs in current clinical use (e.g., Axicabtagene Ciloleucel, Lisocabtagene Maraleucel etc.) comprise a heterologous antigen binding domain (e.g., scFv) operationally linked to the stalk (hinge), transmembrane, co-stimulatory and cytosolic domains derived from multiple different native receptors. In another aspect, the disclosure provides that a SAR (e.g., a next generation CAR) that comprises the native configuration of the transmembrane and cytosolic domains of a naturally occurring signaling receptor (e.g., CD16, NKp30, NKp44, NKp46, NKG2D, KIR2DS4 etc.) or a signaling adaptor (e.g., CD3ƒ, FcRγ, DAP10, DAP12 etc.) shows superior physiological cell signaling and regulation as compared to a SAR comprising non-native configuration of the transmembrane and cytosolic domains of a naturally occurring signaling receptor or a signaling adaptor. In another aspect, the disclosure provides that a SAR that comprises the native configuration of the transmembrane and cytosolic domains of a naturally occurring signaling receptor or a signaling adaptor shows superior in vitro and in vivo efficacy as compared to a SAR comprising non-native configuration of the transmembrane and cytosolic domains of a naturally occurring signaling receptor or a signaling adaptor.

In an embodiment, the SAR comprises one or more heterologous antigen binding domains that are operationally linked via optional linkers to the entire extracellular-, transmembrane- and cytosolic-domains of a naturally occurring signaling receptor or a signaling adaptor or a variant or a fragment thereof. In an embodiment, the SAR comprises one or more heterologous antigen binding domains that are operationally linked via optional linkers to the entire extracellular antigen binding domain, transmembrane- and cytosolic-domains of a naturally occurring signaling receptor or a variant or a fragment thereof. In an embodiment, the SAR comprises one or more heterologous antigen binding domains that are operationally linked via optional linkers to the partial or entire extracellular antigen binding domain of one naturally occurring receptor and the transmembrane- and cytosolic-domains of a different naturally occurring signaling receptor or a variant or a fragment thereof. In an embodiment, the SAR comprises one or more heterologous antigen binding domains that are operationally linked via optional linkers to the partial extracellular domain but the entire transmembrane- and cytosolic-domains of a naturally occurring signaling receptor or a signaling adaptor or a variant or a fragment thereof. In an embodiment, the SAR comprises one or more heterologous antigen binding domains that are operationally linked via optional linkers and/or spacers (e.g., hinge domains) to the transmembrane domain and optionally the cytosolic domain of a naturally occurring signaling receptor or a signaling adaptor or a variant or a fragment thereof. In an embodiment, the SAR comprises one or more heterologous antigen binding domains that are operationally linked via optional linkers to the hinge domain, transmembrane domain and optionally the cytosolic domain of a naturally occurring signaling receptor or a signaling adaptor or a variant or a fragment thereof.

In an embodiment, the different domains of a SAR are operationally linked via peptide bonds, i.e., they are part of a polypeptide chain.

In an embodiment, the SAR comprises an intracellular activation domain. In an exemplary embodiment, the SAR comprises an intracellular activation domain derived from a signaling adaptor. In exemplary embodiments, a SAR comprises intracellular activation domains derived from CD3ƒ, FcRγ, DAP10 or DAP12. In an embodiment, the activation domain of SAR comprises one or more ITAM motifs. In an embodiment, the SAR comprises an activation domain that possesses one or more ITAM motifs. In an embodiment, the SAR comprises an activation domain that possesses two or more ITAM motifs. In an embodiment, the SAR comprises an activation domain that possesses a single ITAM motif. In an embodiment, the SAR comprises an activation domain that lacks an ITAM motifs. In an embodiment, the SAR comprises an activation domain that comprises a tyrosine-based motif (YINM). In an embodiment, the SAR comprises an activation domain that recruits the p85 subunit of PI3K and/or Grb2. In an embodiment, the SAR comprises an activation domain that activates one or more of NFAT, PI3K, NF-κB and ERK signaling pathways.

In an embodiment, the SAR comprises an intracellular inhibitory domain. In an exemplary embodiment, the SAR comprises an intracellular inhibitory domain derived from PD1. In an embodiment, the inhibitory domain of SAR comprises one or more ITIM motifs.

In an embodiment, the SAR is capable of recruiting signaling adaptors. In an exemplary embodiment, the SAR is capable of recruiting one or more signaling adaptors selected from the group of, but not limited to, CD3ƒ, FcRγ, DAP10 and DAP12. In an embodiment, the SAR is capable of recruiting signaling adaptors via interactions with its hinge, transmembrane or cytosolic domains. In an embodiment, the SAR is capable of recruiting signaling adaptors via interactions with one of more of the hinge, transmembrane domain and cytosolic domains. In an embodiment, the immune cells (e.g., T cell, NK cells, macrophages, granulocytes, dendritic cells etc.) expressing the SAR of the disclosure recruit signaling adaptors when their one or more heterologous antigen binding domains bind to the target antigens. In an embodiment, the immune cells (e.g., T cell, NK cells, macrophages, granulocytes, dendritic cells etc.) expressing the SAR of the disclosure activate, proliferate, secrete cytokines and/or modulate (induce or suppress) killing of the target cells and have MHC-restricted and/or MHC-non-restricted antibody-type specificity

In an embodiment, the SAR lacks a cytosolic domain. In an embodiment, the SAR comprise a cytosolic domain that is less than 100 amino acids in length (i.e., less than 90, 80, 70, 60, 50, 50, 40, 30, 25, 20, 15, 10, 5 or 2 amino acids in length. In an embodiment, the SAR comprise a cytosolic domain that is less than 50 amino acids in length. In an embodiment, the SAR comprise a cytosolic domain that is less than 25 amino acids in length. In an embodiment, the SAR comprise a cytosolic domain that is less than 10 amino acids in length. In an embodiment, the SAR comprise a cytosolic domain that is less than 5 amino acids in length. In an embodiment, the SAR lacks an intracellular activation domain. In an embodiment, the SAR lacks an intracellular domain containing ITAM motifs. In an embodiment, the SAR lacks an intracellular signaling domain. In an embodiment, the SAR lacks an intracellular costimulating domain. In an embodiment, the SAR lacks an intracellular domain derived from one or more of CD3z, 2B4, 4-1BB, CD28, ICOS, CD2, CD40, DAP10 and DAP12. In an embodiment, the SAR lacks an intracellular signaling domain that is capable of directly recruiting a protein kinase or a protein phosphatase.

In an embodiment, a SAR lacks a co-stimulatory domain inserted between the transmembrane and the cytosolic domain of a naturally occurring signaling receptor or a signaling adaptor or a variant or a fragment thereof. In contrast to a second-generation CAR, in one embodiment, the transmembrane and cytosolic domains of a SAR of the current disclosure are derived from a single naturally occurring signaling receptor or a signaling adaptor or a variant or a fragment thereof. In another embodiment, the transmembrane and cytosolic domains of a SAR of the current disclosure are derived from a single naturally occurring signaling receptor or a signaling adaptor or a variant or a fragment thereof and are not interrupted by a heterologous co-stimulatory domain derived from a co-stimulatory receptor. In another embodiment, the transmembrane and cytosolic domains of a SAR of the current disclosure are derived from a single naturally occurring signaling receptor or a signaling adaptor and abut each other. In another embodiment, hinge, transmembrane and cytosolic domains of a SAR of the current disclosure are derived from a single naturally occurring signaling receptor or a signaling adaptor or a variant thereof and abut each, i.e., they are present in one continuous chain without interruption.

In an embodiment, the SAR further comprises one or more co-stimulatory domains. In an exemplary embodiment, the SAR comprises one or more co-stimulatory domains derived from CD28, 4-1BB, CD27, OX40, CD2, CD40, CD81 or 2B4 or variants thereof. Other costimulatory domains are known in the art and can be used in alternate embodiments of the disclosure. In an embodiment, the one or more co-stimulatory domains are located in the juxtamembrane region of the SAR. In an embodiment, the one or more co-stimulatory domains are located C-terminus to the transmembrane region of the Type I transmembrane SAR. In an embodiment, the one or more co-stimulatory domains are present N-terminus to the transmembrane region of a type II transmembrane SAR. In another embodiment, the SAR lacks a co-stimulatory domain.

In an embodiment, the disclosure provides a SAR comprising one or more co-stimulatory domains that are inserted between the transmembrane and cytosolic domains derived from a naturally occurring signaling receptor (e.g., CD16A or NKp30 etc.) or a signaling adaptor (e.g., CD3ƒ, FcRγ etc.) or a variant or a fragment thereof. In an exemplary embodiment, the disclosure provides a SAR comprising one or more heterologous antigen binding domains that are operationally linked to the extracellular domain derived from a naturally occurring signaling receptor (e.g., CD16A, CD16B, CD64, NKp30 etc.), which in turn is linked to a hinge domain (e.g., CD8 hinge or CD28 hinge), a transmembrane domain (e.g., CD8 or CD28 transmembrane domain), a costimulatory domain (e.g., 4-1BB or CD28 co-stimulatory domain) and an activation domain (e.g., CD3ƒ or FcRγ activation domain). An exemplary such SAR is represented by SEQ ID NO:10818 and 10821. In another exemplary embodiment, the disclosure provides a SAR comprising one or more heterologous antigen binding domains that are operationally linked to the extracellular hinge domain derived from a signaling adaptor (e.g., CD3ƒ, FcRγ etc.), which in turn is linked to a transmembrane domain (e.g., CD3ƒ or FcRγ transmembrane domain), a costimulatory domain (e.g., 4-1BB, CD28, 2B4 or OX40 co-stimulatory domain) and an activation domain (e.g., CD3ƒ or FcRy activation domain etc.).

In an embodiment, SAR comprises one or more heterologous antigen binding domains (e.g., scFv, vHH, FHVH, centyrin, scTCR, svd-TCR etc.) that are operationally linked via optional linkers to the amino-terminus or near the amino-terminus of the extracellular domain of a naturally occurring (or native) signaling receptor or a signaling adaptor or a variant or a fragment thereof. In an embodiment, SAR comprises one or more heterologous antigen binding domains that are operationally linked via optional linkers to the amino-terminus or near the amino-terminus of the hinge (or spacer) domain of a naturally occurring signaling receptor or a signaling adaptor or a variant or a fragment thereof. In an embodiment, SAR comprises one or more heterologous antigen binding domains that are operationally linked via optional linkers to the amino-terminus or near the amino-terminus of the transmembrane domain of a naturally occurring signaling receptor or a signaling adaptor or a variant or a fragment thereof. In an embodiment, SAR comprises one or more heterologous antigen binding domains that are operationally linked via optional linkers to the hinge (spacer) domain, the transmembrane domain and cytosolic domain of a naturally occurring signaling receptor or a signaling adaptor or a variant or a fragment thereof. In an embodiment, SAR comprises one or more heterologous antigen binding domains that are operationally linked via optional linkers to the transmembrane domain and the cytosolic domain of a naturally occurring signaling receptor or a signaling adaptor or a variant or a fragment thereof.

In an exemplary embodiment, the naturally occurring (or endogenous) signaling receptor comprising a SAR is a type I (or group 1) transmembrane protein with its N-terminus on the extracellular side and C-terminus on cytosolic side. Exemplary such type I (or group 1) endogenous receptors include CD16A, NKp30, NKp44, NKp46, KIR2DL1, KIR2DL2, KIR2DL3, KIR2DL5A, KIR2DL5B, KIR3DL1, KIR3DL2, KIR3DL4, KIR2DL4, KIR2DS1, KIR2DS2, KIR2DS3, KIR2DS4, KIR2DS5, KIR3DS1, CRTAM, TIGIT, CD96, 2B4, SLAMF6, SLAMF7, CD27, CD100, CD160, ILT2/LILRB1, CD33, SIGLEC-7, SIGLEC-9, CD32 and CD64 etc.

In one embodiment, the disclosure provides a synthetic antigen receptor (SAR) comprising one or more heterologous antigen binding domains that are operationally linked to the amino-terminus or near the amino-terminus of a type I transmembrane naturally occurring (or native) signaling receptor via an optional linker. In an embodiment, the SAR retains the binding capability and function of the naturally occurring signaling receptor but in addition acquires the binding capability conferred by the one or more heterologous antigen binding domains. In an embodiment, the one or more heterologous antigen binding domains comprise scTCR, svd-TCR or a TCR-mimic antibody or fragment thereof. In some embodiments, the SAR acquires TCR like binding capabilities. In an embodiment, SAR comprises one or more heterologous antigen binding domains that are operationally linked to the amino-terminus or near the amino-terminus of an endogenous receptor that is a type I transmembrane protein. In an embodiment, the SAR comprises a heterologous (non-natural) antigen binding domain that is operationally linked via an optional linker to the N-terminus or near the N-terminus of CD16A, CD16B, NKp30, NKp44, NKp46, KIR2DL1, KIR2DL2, KIR2DL3, KIR2DL5A, KIR2DL5B, KIR3DL1, KIR3DL2, KIR3DL4, KIR2DL4, KIR2DS1, KIR2DS2, KIR2DS3, KIR2DS4, KIR2DS5, KIR3DS1, CRTAM, TIGIT, CD96, 2B4, SLAMF6, SLAMF7, CD27, CD100, CD160, ILT2/LILRB1, CD33, SIGLEC-7, SIGLEC-9, CD32 or CD64. In an embodiment, the SAR also comprises an N-terminal signal peptide. In an embodiment, the SAR comprises one or more antigen binding domains that are located C-terminus to a signal peptide.

In an embodiment, a SAR comprises one or more heterologous antigen binding domains that are operationally linked via one or more optional linkers to a polypeptide comprising the hinge (spacer), transmembrane and cytosolic domains of CD16A, CD16B, NKp30, NKp44, NKp46, KIR2DL1, KIR2DL2, KIR2DL3, KIR2DL5A, KIR2DL5B, KIR3DL1, KIR3DL2, KIR3DL4, KIR2DL4, KIR2DS1, KIR2DS2, KIR2DS3, KIR2DS4, KIR2DS5, KIR3DS1, CRTAM, TIGIT, CD96, 2B4, SLAMF6, SLAMF7, CD27, CD100, CD160, ILT2/LILRB1, CD33, SIGLEC-7, SIGLEC-9, CD32 or CD64.

In an exemplary embodiment, the naturally occurring (or endogenous) signaling receptor is a type II (or group 2) transmembrane protein with its C-terminus on the extracellular side and N-terminus on cytosolic side. The disclosure provides a general method for generating a fusion protein between a Type I membrane protein and a Type II membrane protein to generate a fusion in which one or more modules of a Type I membrane protein are functionally linked to the entire or partial region of a type II membrane protein. In an exemplary embodiment, N-terminus of the antigen binding domain of a type I protein lacking the signal peptide sequence is operationally linked via an optional linked to the C-terminus of a type II membrane protein. In an embodiment, a SAR comprises one or more heterologous antigen binding domains that are operationally linked via one or more optional linker to the carboxy-terminus or near the carboxy-terminus of a naturally occurring (or endogenous) signaling receptor. Exemplary such type II (or group 2) endogenous receptors include, but are not limited to, NKG2D, NKG2A, NKG2C, NKG2F, NKG2E, NKG2H, KLRG1, CD161 and CD94 etc. In an embodiment, the one or more heterologous antigen binding domains (e.g., vHH, FHVH, centyrin, scTCR, svd-TCR) are operationally linked in frame via one or more optional linkers to the C-terminus or near the C-terminus of an endogenous receptor that is a type II (group 2) transmembrane protein. In an embodiment, a SAR comprises one or more heterologous antigen binding domains that are operationally linked via optional linkers to the C-terminus or near the C-terminus of NKG2D, NKG2A, NKG2C, NKG2F, NKG2E, NKG2H, KLRG1, CD161 or CD94. Exemplary such SARs comprising a Her2 and Her3 vHH domains attached to the C-terminus of NKG2D are represented by SEQ ID NO (DNA):7696-7697 and SEQ ID NO (PRT):8388-8389, respectively. In an embodiment, the one or more heterologous antigen binding domains are operationally linked via optional linkers to the hinge domain of an endogenous receptor that is a type II (group 2) transmembrane protein. In an embodiment, the one or more heterologous antigen binding domains are operationally linked via optional linkers to the hinge domain of NKG2D, NKG2A, NKG2B, NKG2C, NKG2F, NKG2E, NKG2H, KLRG1, CD161 or CD94.

Efficient expression of NKG2D on the cell surface requires the presence of DAP10. Provided herein is a strategy to obtain effector cells stably overexpress an NKG2D-SAR (i.e., a SAR comprising the transmembrane domain and/or cytosolic domain of NKG2D) alone or along with DAP10 by genetically engineering a cell (e.g., iPSC) to introduce NKG2D-SAR, and optionally DAP10 to the cell (e.g., iPSC), and then derive effector cells including NK and T cells from directed iPSC differentiation. The disclosure also provides a strategy for efficient expression of an NKG2D-SAR in a cell that lacks DAP10 or expresses low levels of DAP10 by ectopic expression of DAP10 as an accessory module along with a SAR comprising NKG2D transmembrane domain.

CD94/NKG2C is a heterodimeric receptor that binds to HLA-E and associates with DAP12, a protein containing an immunoreceptor tyrosine-based activating motif. Efficient expression of CD94/NKG2C on the cell surface requires the presence of DAP 12 and charged amino acids in the transmembrane domains of DAP 12 and NKG2C mediate this interaction. Provided herein is a strategy to obtain effector cells stably overexpress an NKG2C-SAR (i.e., a SAR comprising the transmembrane domain of NKG2C) alone or along with DAP12 by genetically engineering a cell (e.g., iPSC) to introduce NKG2C-SAR, and optionally DAP12 to the cell (e.g., iPSC), and then derive effector cells including NK and T cells from directed iPSC differentiation. In some embodiments, NKG2C-SAR is further co-expressed with CD94 or a CD94-SAR.

In an iPSCs or an effector cell derived therefrom comprising an overexpressed NKG2C-SAR, the cell further comprises overexpressed CD94 or CD94-SAR and/or DAP12. In one embodiment, NKG2C-SAR and CD94 (or a CD94-SAR) and/or DAP12 are expressed in separate constructs. In another embodiment, NKG2C-SAR and CD94 (or a CD94-SAR) and/or DAP12 are co-expressed in a bi-cistronic or tri-cistronic construct and are linked by a self-cleaving 2A coding sequence. In another embodiment, NKG2C-SAR, CD94 (or CD94-SAR) and DAP 12 are expressed in separate constructs.

The disclosure provides that a SAR comprising the transmembrane domain of NKG2A, NKG2B, NKG2C, NKG2E, NKG2F, NKG2H can be similarly efficiently expressed by co-expression of CD94 and/or signaling adaptors (e.g., DAP10, DAP12 etc.) that are known to associate with them.

In one embodiment, the disclosure provides a SAR comprising one or more heterologous antigen binding domains that are operationally linked via optional linkers to the carboxy-terminus, or near the carboxy-terminus of a type II transmembrane naturally occurring signaling receptor. In an embodiment, the SAR retains the binding capability and function of the natural occurring signaling receptor but in addition acquires the binding capability conferred by the heterologous antigen binding domain. In one embodiment, the disclosure provides one or more heterologous antigen binding domains that are operationally linked in frame via optional linkers to the hinge domain or transmembrane domain of a type II transmembrane naturally occurring signaling receptor to generate a synthetic antigen receptor. In an embodiment, the resulting SAR retains the binding capability and function of the native signaling receptor but in addition acquires the binding capability conferred by the heterologous antigen binding domain. In an embodiment, a SAR of the disclosure retains the binding properties and physiological regulation of a naturally occurring receptor while acquiring additional antigen binding capabilities that allows it to respond to target antigens in addition to those to which the naturally occurring receptor can respond.

The disclosure also provides single-chain SARs comprising one or more heterologous antigen binding domains that are operationally linked via optional linkers to the extracellular domain of a naturally occurring signaling chain or a signaling adaptor or a variant thereof. The disclosure also provides single-chain SARs comprising one or more heterologous antigen binding domains that are operationally linked via optional linkers to the hinge (spacer) domain of a naturally occurring signaling chain or a signaling adaptor or a variant thereof. The disclosure also provides single-chain SARs comprising one or more heterologous antigen binding domains that are operationally linked via optional linkers to the transmembrane domain of a naturally occurring signaling chain or a signaling adaptor or a variant thereof. Exemplary signaling chains or signaling adaptors include but are not limited to CD3ƒ, FcRγ, DAP10 or DAP12 or variants thereof. In an embodiment, the signaling chain/adaptor further comprises a co-stimulatory domain.

In an embodiment, the disclosure provides a SAR comprising one or more heterologous antigen binding domains that are operationally linked via optional linkers to the entire or partial extracellular domain, hinge domain or transmembrane domain of a NCAM (non CD3 adaptor module).

In an embodiment, the disclosure provides a SAR comprising one or more heterologous antigen binding domains that are operationally linked to the entire or partial extracellular domain, hinge domain or transmembrane domain of a signaling adaptor via optional linkers wherein the signaling adaptor (or a signaling chain) is not CD3R, CD3γ, CD36, CD3ƒ. In an embodiment, the signaling adaptor is not FcRγ.

In an embodiment, the one or more heterologous antigen binding domains of a single chain SAR comprise an autonomous antigen binding domains (AABD), e.g., a single domain antibody, single vH domain, FHVH, vHH domain, svd-TCR, non-immunoglobulin antigen binding scaffold (e.g., DARPIN, an affibody, an affilin, an adnectin, an affitin, an obodies, a repebody, a fynomer, an alphabody, an avimer, an atrimer, a centyrin, a pronectin, an anticalin, a kunitz domain, an Armadillo repeat protein or a fragment thereof). In an embodiment, the one or more heterologous antigen binding domains of a single chain SAR comprise an antibody or antibody fragment (e.g., vL, vH, Fab, Fab′2, scFv and scTCR etc.).

As SARs are modular in format, their different domains can be replaced by other domains to generate new SARs with diverse biological activities and properties. Thus, the extracellular domain, hinge domain, transmembrane domain and/or cytosolic domain of one SAR can be substituted by the extracellular domain, hinge domain, transmembrane domain and/or cytosolic domain of another SAR as long as the resulting SAR possesses at least one of the biological activities (e.g., antigen binding, cell signaling etc.) of the original SARs. In an embodiment, the disclosure provides that new modules (co-stimulatory domains) can be inserted in a SAR. In an embodiment, the disclosure provides a SAR comprising one more heterologous antigen binding domains that are operationally linked via optional linkers to the entire or partial extracellular domain of one naturally occurring signaling receptor, which in turn, is operationally linked to the transmembrane and cytosolic domain of a different naturally occurring receptor or a variant thereof. In an exemplary embodiment, the extracellular domain of a SAR comprising the CD16A extracellular, transmembrane and cytosolic domains is replaced by the extracellular domain of CD64 to generate a new SAR (SEQ ID NO: 4722) comprising a CD20 vHH domain, a CD64 extracellular domain, CD16A transmembrane domain and CD16A cytosolic domain.

In an embodiment, the different domains of a SAR are derived from a single naturally occurring receptor or signaling adaptor. In an embodiment, the different domains of a SAR are derived from more than one naturally occurring receptor or signaling adaptor. In an embodiment, the SAR comprises one or more heterologous antigen binding domains that are operationally linked via optional linkers to the partial or entire antigen binding domain of one naturally occurring receptor and the hinge, transmembrane- and cytosolic-domains derived from one or more different receptors or variants or fragments thereof. In an exemplary embodiment, a SAR comprises from N to C terminus a CD19 scFv, a CD16 antigen binding domain (D1 and D2), a CD8 hinge domain, a CD8 transmembrane domain, a 4-1BB co-stimulatory domain and a CD3z activation domain. The amino acid sequence of such a SAR is presented in SEQ ID NO: 10836. In another exemplary embodiment, a SAR comprises from N to C terminus a CD19 scFv, a CD16A antigen binding domain (D1 and D2), a CD16A hinge domain, a CD28 transmembrane domain, a CD28 co-stimulatory domain and a CD3z activation domain. In another exemplary embodiment, a SAR comprises from N to C terminus a CD19 scFv, a CD64 antigen binding domain, a CD16A hinge domain, a CD16A transmembrane domain, a CD16A cytosolic domain. The amino acid sequence of such a SAR is presented in SEQ ID NO: 10832.

In an embodiment, SAR comprises one or more heterologous antigen binding domains that are operationally linked via optional linkers to the hinge domain, the transmembrane domain and the cytosolic domain of a naturally occurring signaling receptor or a variant thereof where the hinge, the transmembrane domain and the cytosolic domains are all derived from a single naturally occurring signaling receptor or a signaling adaptor or a variant or a fragment thereof. In an embodiment, the hinge domain, the transmembrane domain and the cytosolic domains comprising the SIR are derived from a single naturally occurring signaling receptor (e.g., CD16A) or a signaling adaptor (e.g., CD3ƒ) or a variant or a fragment thereof. In an alternate embodiment, the hinge domain, the transmembrane domain and the cytosolic domains comprising the SIR are derived from more than one naturally occurring signaling receptors (e.g., hinge and transmembrane domains of CD16A are operationally linked to the cytosolic domain of NKp30 etc.) or signaling adaptors (e.g., hinge and transmembrane domains of CD16A are operationally linked to the cytosolic domain of FcRγ etc.). In an embodiment, SAR comprises one or more heterologous antigen binding domains that are operationally linked via optional linkers to the hinge domain, the transmembrane domain and the cytosolic domain of a naturally occurring signaling receptor where the hinge, the transmembrane domain and the cytosolic domains are derived from more than one naturally occurring signaling receptor or a signaling adaptor or a variant or a fragment thereof.

In an embodiment, the disclosure provides a method for generating a non-native protein (i.e., a synthetic protein) comprising two or more chains with the following general formula from amino (N) to carboxy (C) termini:
Chain 1: SP1-A1-L1-H1-M1-(C1)n
Chain 2: SP2-A2-L2-H2-M2-(C2)n

Where SP1 and SP2 are optional signal peptides that are cleaved from the mature polypeptide chains; A1 and A2 are two protein domains that can interact with each other, L1 and L2 are optional linkers, H1 and H2 are optional hinge or spacer domains, M1 and M2 are membrane-anchoring or transmembrane domains and C1 and C2 are optional cytosolic domains. In an embodiment, the A1 and A2 domains are not derived from antibodies. In an embodiment, the A1 and A2 domains are not antibody fragments. In an embodiment, A1 and A2 domains are heterologous to M1 and M2 domains, i.e., A1 and M1 domains are derived from different proteins and similarly A2 and M2 domains are derived from different proteins. In an exemplary embodiment, A1 and A2 domains are derived from a TCR (e.g., Vα and VP domains of a TCR) and M1 and M2 domains are derived from CD3ƒ. In an embodiment, A1 and A2 domains are not autonomous domains. In an embodiment, A1 and A2 domains have affinity for each other that is greater than their affinity for an irrelevant protein. In an embodiment, A1 and A2 domains may associate with each other to generate an antigen binding domain. In an embodiment, the non-native protein is a synthetic antigen receptor. In an embodiment, L1 and L2 linkers are long linkers. In an embodiment, L1 and L2 linkers are Ig like linkers. In an embodiment, L1 and L2 linkers are joined by one or more disulfide bonds. In an embodiment, M1 and M2 domains are transmembrane domains. In an embodiment, M1 and M2 domains are derived from the same protein (e.g., CD3ƒ). In an embodiment, M1 and M2 domains are derived from different proteins (e.g., CD3ƒ and FcRy). In an embodiment, M1 and M2 domains are identical in sequence and/or possess greater than 70%, (e.g., 75%, 80%, 85%, 90%, 95%, 98%, 99%, 99.9% etc.) amino acid sequence homology. In an embodiment, M1 and M2 domains associate with each other. In an embodiment, M1 and M2 domains are joined by a disulfide bond. In an embodiment, M1 and/or M2 domains can recruit one or more signaling adaptors. In an embodiment, C1, C2 domains are cell signaling domains (e.g., activation domain or co-stimulatory domain etc.). In an embodiment, each chain may possess more than one cytosolic domain. In an embodiment, both chains are expressed on the cell surface where the A1-L1-H1 and A2-L2-H2 segments are located on the extracellular side. The disclosure provides nucleic acid, amino acid sequence encoding the synthetic protein, one or more vectors encoding the synthetic protein and cells expressing the synthetic protein.

The disclosure also provides a double chain SAR (such as an isolated double chain SAR). In one embodiment, the disclosure provides a double chain SAR comprising one or more heterologous antigen binding domains that are operationally linked via optional linkers to at least one module comprising the entire or partial extracellular domain, transmembrane domain and optionally the cytosolic domain of a signaling receptor, or a signaling adaptor or a variant or a fragment thereof. In an embodiment, the signaling receptor is a non-TCR signaling receptor. In an embodiment, the signaling adaptor is a non-CD3 adaptor. In an embodiment, the signaling adaptor is not CD3ƒ.

In one embodiment, the disclosure provides a double chain SAR comprising two chains each of which comprises at least one antigen binding domain (e.g., vL, vβ, Vα, Vβ, Vγ or Vδ etc.) that is operationally linked via an optional peptide linker (e.g., IgCL, IgCH1 etc.) to a membrane associated module (MAM) comprising the transmembrane domain or membrane associated domain of a signaling receptor (e.g., CD16A, CD16B, NKp30 etc.), or a signaling adaptor (e.g., CD3ƒ, FcRy) or a variant or a fragment thereof. In some embodiments, the MAM further comprises the entire or partial extracellular antigen binding domain, the hinge domain and/or the cytosolic domain of a signaling receptor and/or the hinge and/or cytosolic domain of a signaling adaptor. In an embodiment, the signaling receptor is a non-TCR signaling receptor. In an embodiment, the module is an NTCRM. In an embodiment, the signaling adaptor is a non-CD3 adaptor (i.e., NCAM). In an embodiment, the signaling adaptor is not CD3ƒ. In an embodiment, the MAM does not comprise the transmembrane domain of TCRα, β, γ, δ, preTCRα, CD3ƒ, CD3ε, CD3γ or CD3δ. In some embodiments, at least one antigen binding domain (e.g., vL, Vα, or Vγ) of the first chain associates with at least one complementary antigen binding domain (e.g., vH, Vβ or Vδ) of the second chain to form an antigen binding module (e.g., Fv or Fc-TCR) that specifically binds to a target antigen. In some embodiments, the first polypeptide chain and the second polypeptide chain are linked via one or more disulfide bonds. In some embodiments, the first peptide linker and the second peptide linker are linked via one or more disulfide bonds. In some embodiments, the first peptide linker and/or the second peptide linker are, individually, from about 5 to about 500 amino acids in length. In an embodiment, the first and the second antigen binding domains comprise complementary chains (e.g., vL and vH, Vα and Vβ or Vγ and Vδ). In some embodiments, the first and the second polypeptide chains further comprise one or more autonomous antigen binding domains (AABD) that are attached to the N-terminus or near the N-terminus of the first (e.g., vL, Vα or Vγ) and/or the second (e.g., vH, Vβ or Vδ) antigen binding domains. In some embodiments, the target antigen is a cell surface antigen. In some embodiments, the cell surface antigen is selected from the group consisting of protein, carbohydrate, and lipid. In an exemplary embodiment, the target antigen is one or more of the antigens listed in Table B. In exemplary embodiments, the cell surface antigen is selected from the group of but not limited to one or more of: CD2, CD5, CD19, CD20, CD22, CD33, CD70, CD123, CD138, CD179b, CLL-1, FLT3, Claudin 18.2, BCMA, GCC, MPL, SLAMF7, ROR1, ROR2, GPRC5D, FCRL5, MSLN, EGFR, EGFRviii, PSMA, PSCA, KLK2, IL13Ra2, TROP2, PTK7, DLL3, Mucd, Mucl6 or Her2. In some embodiments, the target antigen is a complex comprising a peptide and a major histocompatibility complex (MHC) protein. In an exemplary embodiment, the peptide antigen is one or more of the antigens listed in Table B. In exemplary embodiments, the peptide/MHC complex comprises a peptide derived from one or more of NY-ESO-1, MAGE-A2, MAGE-A3, MAGE4, WT1, AFP, TERT, MART-1, pp66-CMV, HPV16-E7, PRAME, EBV-LMP2A, HIV-1, PSA or gp100.

In one embodiment, the disclosure provides a double chain SAR comprising two chains each of which comprises one or more heterologous antigen binding domains that are operationally linked via optional linkers to a module comprising the hinge, transmembrane and optionally the cytosolic domain of a signaling receptor, or a signaling adaptors or a variant or a fragment thereof. In one embodiment, the disclosure provides a double chain SAR comprising two chains each of which comprises one or more heterologous antigen binding domains that are operationally linked via optional linkers to a module comprising the transmembrane and optionally the cytosolic domain of a signaling receptor, a signaling adaptor or a variant or a fragment thereof. In one embodiment, the disclosure provides a double chain SAR comprising two chains each of which comprises one or more heterologous antigen binding domains that are operationally linked via optional linkers to a module comprising the transmembrane domain of a signaling receptor, or a signaling adaptor or a variant or a fragment thereof. In one embodiment, the disclosure provides a double chain SAR comprising two chains each of which comprises one or more heterologous antigen binding domains that are operationally linked via optional linkers to a module comprising the cytosolic domain of a signaling receptor, a signaling adaptors or variant or a fragment thereof. In an embodiment, the signaling receptor is a non-TCR signaling receptor. In an embodiment, the module is an NTCRM. In an embodiment, the signaling adaptor is a non-CD3 adaptor (i.e., NCAM). In an embodiment, the signaling adaptor is not CD3ƒ.

In one embodiment, the disclosure provides a double chain SAR comprising one or more heterologous antigen binding domains where each chain is operationally linked via optional linkers to a module (e.g., NTCRM) comprising the extracellular, transmembrane or the cytosolic domain of a signaling receptor (e.g., native receptors), a signaling adaptor (e.g., NCAM) or a variant or a fragment thereof. In an embodiment, the signaling receptor and signaling adaptors comprising a double chain SAR are naturally occurring. In an embodiment, the signaling receptor and signaling adaptors comprising a double chain SAR are non-naturally occurring. In an embodiment, the signaling receptor and signaling adaptors comprising a double chain SAR are non-T cell receptors and non-CD3 adaptors. In an embodiment, the signaling receptor and signaling adaptors comprising a double chain SAR are non-T cell receptors and non-CD3 adaptors that are naturally occurring.

In one embodiment, the disclosure provides a double chain SAR comprising one or more heterologous antigen binding domains that are operationally linked via optional linkers to at least one module (e.g., NTCRM) comprising the extracellular, transmembrane or the cytosolic domain of a naturally occurring signaling receptor (e.g., CD16A), a signaling adaptor (e.g., FcRy) or a variant or a fragment thereof.

The present application in one aspect provides a construct (such as an isolated construct) comprising one or more heterologous antigen binding domains fused to a non-T cell receptor module (NTCRM). In an exemplary embodiment, a NTCRM is derived from but not limited to one or more of the following non-TCR receptors: CD16A, CD16B, CD64, CD32, NKp30, NKp44, NKp46, KIR2DL1, KIR2DL2, KIR2DL3, KIR2DL5A, KIR2DL5B, KIR3DL1, KIR3DL2, KIR3DL4, KIR2DL4, KIR2DS1, KIR2DS2, KIR2DS3, KIR2DS4, KIR2DS5, KIR3DS1, DNAM-1, 2B4, OX40, CD28, 4-1BB, CD27, CD81, CD2, CD5, TNFR-I, TNFR-II, Fas, CD30, CD40, CRTAM, TIGIT, CD96, SLAMF6, SLAMF7, CD100, CD160, and ILT2.

In some embodiments, the SAR comprises one or more heterologous antigen-binding domains that specifically bind to a target antigen and a non-T cell receptor module (NTCRM) capable of recruiting at least one signaling adaptor. In an embodiment, the signaling adaptor is a non-CD3 adaptor (i.e., NCAM). In some embodiments, the target antigen is a complex comprising a peptide and an MHC protein (such as an MHC class I protein or an MHC class II protein). In some embodiments, the target antigen is a cell-surface antigen.

In some embodiments, there is provided a SAR (such as an isolated SAR) that specifically binds to a target antigen, wherein the SAR comprises: a) a first polypeptide chain comprising a first antigen-binding domain comprising a vL, a Vα or a Vγ domain and a first Membrane associated module (MAM); and b) a second polypeptide chain comprising a second antigen-binding domain comprising a vH, a Vβ or a Vδ domains and a second Membrane associated module (MAM), wherein the vL, Vα or Vγ domain of the first antigen-binding domain and the complementary vH, Vβ or Vδ domain of the second antigen-binding domain form a Fv or TCR-Fv like antigen-binding module that specifically binds to the target antigen, and wherein the first MAM and the second MAM form a non-T cell receptor module (NTCRM), and wherein the NTCRM is capable of activating at least one signaling pathway and/or recruiting at least one signaling adaptor. In an embodiment, the first MAM and the second MAM do not comprise the transmembrane domain of a TCR chain selected from TCRα, TCRβ, TCRγ, TCRδ or preTCRα. In an embodiment, the first MAM or the second MAM do not comprise the transmembrane domain of a TCR chain selected from TCRα, TCRβ, TCRγ, TCRδ or preTCRα. In an embodiment, the NTCRM does not comprise the transmembrane domain of two TCR chain selected from i) TCRα and TCRβ, ii) TCRγ and TCRδ, or iii) preTCRα and TCRβ. In an embodiment, the first MAM and the second MAM do not comprise the transmembrane domain of a CD3 chain selected from CD3R, CD3γ, CD3δ or CD3ƒ. In an embodiment, the first MAM and the second MAM do not comprise the transmembrane domain of a TCR chain and a CD3 chain. In an embodiment, the first MAM and the second MAM do not comprise the transmembrane domain of CD3ƒ. In an exemplary embodiment, a NTCRM is derived from but not limited to one or more of the following non-TCR receptors: CD16A, CD16B, CD64, CD32, NKp30, NKp44, NKp46, KIR2DL1, KIR2DL2, KIR2DL3, KIR2DL5A, KIR2DL5B, KIR3DL1, KIR3DL2, KIR3DL4, KIR2DL4, KIR2DS1, KIR2DS2, KIR2DS3, KIR2DS4, KIR2DS5, KIR3DS1, DNAM-1, 2B4, OX40, CD28, 4-TBB, CD27, CD81, CD2, CD5, TNFR-I, TNFR-II, Fas, CD30, CD40, CRTAM, TIGIT, CD96, SLAMF6, SLAMF7, CD100, CD160, and ILT2. In some embodiments, the signaling adaptor is selected from but not limited to one or more of CD3ƒ, FcRγ, DAP10 and/or DAP12 or variants or fragments thereof. In some embodiments, the signaling adaptor is a non-CD3 adaptor (NCAM). In some embodiments, the signaling adaptor is not CD3ƒ. In some embodiments, the first polypeptide chain and the second polypeptide chain are linked via one or more disulfide bonds. In some embodiments, the first polypeptide chain further comprises a first peptide linker between the first antigen-binding domain and the first MAM. In some embodiments, the second polypeptide chain further comprises a second peptide linker between the second antigen-binding domain and the second MAM. In some embodiments, the first polypeptide chain and the second polypeptide chain are linked via one or more disulfide bonds. In some embodiments, the first peptide linker and/or the second peptide linker are, individually, from about 5 to about 500 amino acids in length. In some embodiments, the first and/or second peptide linkers comprise, individually, a constant domain or fragment thereof from an immunoglobulin or T cell receptor subunit. In some embodiments, the first and/or second peptide linkers comprise, individually, a CH1, CH2, CH3, CH4 or CL antibody domain, or a fragment thereof. In some embodiments, the first and/or second peptide linkers comprise, individually, a Cα, Cβ, Cγ, or Cδ TCR domain, or a variant or a fragment thereof. In exemplary embodiments, the first and/or second linkers comprise, individually, an Ig like linker (e.g., IgCL, IgCH1 etc.) derived from an immunoglobulin (e.g., SEQ ID NO: 3536-3551) or a TCR-Ig like linker (e.g., TCRb-Ig3, SEQ ID NO: 3560; TCRa-Ig3, SEQ ID NO:3562; TCRg-Ig3, SEQ ID NO: 3566; or TCRd-Ig3, SEQ ID NO: 3568 etc.) or a variant or a fragment thereof. In some embodiments, a vL domain is attached to an IgCL linker and a vH domain is attached to an IgCHT linker. In some embodiments, a vL domain is attached to an IgCHT linker and a vH domain is attached to a IgCL linker. In some embodiments, a Vα domain is attached to a Cu-derived linker (e.g., TCRa-Ig3) and Vβ domain is attached to a Cβ derived linker (e.g., TCRb-Ig3). In some embodiments, a Vβ domain is attached to a Cu-derived linker (e.g., TCRa-Ig3) and Vα domain is attached to a Cβ derived linker (e.g., TCRb-Ig3). In some embodiments, a Vγ domain is attached to a Cγ-derived linker (e.g., TCRg-Ig3) and Vδ domain is attached to a Cδ derived linker (e.g., TCRd-Ig3). In some embodiments, a Vγ domain is attached to a Cδ-derived linker (e.g., TCRd-Ig3) and Vδ domain is attached to a Cy derived linker (e.g., TCRg-Ig3). In some embodiments, other configurations of variable domains and linkers are envisioned. In some embodiments, first and/or second peptide linkers comprise mutations that increase the expression, affinity and the pairing of the two polypeptide chains. In an embodiment, the first and the second antigen binding domains comprise complementary chains (e.g., vL and vH, Vα and Vβ or Vγ and Vδ). In some embodiments, the first and the second polypeptide chains further comprise one or more autonomous antigen binding domains (AABD) that are attached to the N-terminus or near the N-terminus of the first (e.g., vL, Vα or Vγ) and/or the second (e.g., vH, Vβ or Vδ) antigen binding domains. In some embodiments, the AABD is selected from one or more of, but not limited to, a single vH domain (SVH), a single vL domain (SVL), a vHH domain, a single domain antibody, a single variable domain of a TCR (svd-TCR), a non-immunoglobulin antigen binding scaffold, a ligand-binding domain of a receptor, a receptor-binding domain of a ligand, an autoantigen, an adaptor binding domain, an Fc binding domain, or a fragment or a variant thereof.

In some embodiments, the SAR binds to the target antigen with an equilibrium dissociation constant (Kd) from about 0.1 pM to about 500 nM. In some embodiments, the target antigen is a cell surface antigen. In an exemplary embodiment, the target antigen is one or more of the antigens listed in Table B. In some embodiments, the cell surface antigen is selected from the group consisting of protein, carbohydrate, and lipid. In some embodiments, the cell surface antigen is one or more of CD2, CD5, CD19, CD20, CD22, CD33, CD70, CD123, CD138, CD179b, CLL-1, FLT3, Claudin 18.2, BCMA, GCC, MPL, SLAMF7, ROR1, ROR2, GPRC5D, FCRL5, MSLN, EGFR, EGFRviii, PSMA, PSCA, KLK2, IL13Ra2, TROP2, PTK7, DLL3, Mucd, Muc16 or Her2. In some embodiments, the target antigen is a complex comprising a peptide and a major histocompatibility complex (MHC) protein. In exemplary embodiments, the peptide/MHC complex comprises a peptide derived from one or more of NY-ESO-1, MAGE-A2, MAGE-A3, MAGE4, WT1, AFP, TERT, MART-1, pp66-CMV, HPV16-E7, PRAME, EBV-LMP2A, HIV-1, PSA or gp100.

In an embodiment, the vL and vH domains of a SAR are derived from a TCR mimic antibody that can recognize intracellular peptides in an MHC-dependent manner. In an embodiment, the Vα and Vβ domains of a SAR are derived from an HLA-independent TCR that can recognize cell surface proteins. In an embodiment, a SAR is bispecific or multispecific. In an embodiment, the disclosure provides a SAR that can bind to two or more antigens that are MHC restricted. In an embodiment, a SAR can bind to two or more antigens that are MHC restricted and/or MHC-non-restricted. In an embodiment, a SAR can bind to a peptide/MHC complex via its Fv or TCR-Fv domain and bind to one or more peptide/MHC complexes via one or more svd-TCR that are attached to the N-terminus or near the N-terminus of its vL and vH, Vα and Vβ or Vγ and Vδ domains. In an embodiment, a SAR can bind to one or more peptide/MHC complex via its Fv, TCR-Fv domain and/or svd-TCR domain and bind to one or more surface antigens via one or more AABD (e.g., vHH, FHVH, centyrin etc.) that are attached to the N-terminus or near the N-terminus of its Vα and Vβ or Vγ and Vδ domains.

In some embodiments, according to any of the SARs (such as isolated SARs) described above, the first polypeptide further comprises a first hinge domain (or connecting peptide) or fragment thereof N-terminal to the first MAM (e.g., transmembrane domain), and/or the second MAM further comprises a second hinge domain (or connecting peptide) or fragment thereof N-terminal to the second MAM (e.g., transmembrane domain). In some embodiments, the SAR comprises a disulfide bond between a residue in the first MAM and the second MAM and/or a disulfide bond between a residue in the first hinge domain and a residue in the second hinge domain. In some embodiments, according to any of the SARs (such as isolated SARs) described above, the first MAM further comprises a first homologous antigen binding domain or fragment thereof N-terminal to the first hinge domain and/or the second polypeptide further comprises a second homologous antigen binding domain or fragment thereof N-terminal to the second hinge domain. In an embodiment, the two homologous antigen binding domains are derived from the same non-T cell receptor as the two hinge domains. In some embodiments, the first MAM further comprises a first cytosolic domain C-terminal to the first transmembrane domain. In some embodiments, the second MAM further comprises a second cytosolic domain C-terminal to the second transmembrane domain. In an embodiment, the first and/or second cytosolic domains are activation domains comprising one or more ITAMs. In some embodiments, the SAR binds to the target antigen with an equilibrium dissociation constant (Kd) from about 0.1 pM to about 500 nM.

In some embodiments, according to any of the SARs (such as isolated SARs) described above, the first polypeptide chain further comprises a first co-stimulatory domain C-terminal to the first transmembrane domain. In some embodiments, the second polypeptide chain further comprises a second co-stimulatory domain C-terminal to the second transmembrane domain. In some embodiment, according to any of the SARs (such as isolated SARs) described above, the first polypeptide chain comprises more than one co-stimulatory domains C-terminal to the first transmembrane domain and/or the second polypeptide chain comprises more than one co-stimulatory domains C-terminal to the second transmembrane domain. In some embodiments, the first polypeptide chain further comprises a first signaling peptide N-terminal to the first antigen-binding domain. In some embodiments, the second polypeptide chain further comprises a second signaling peptide N-terminal to the second antigen-binding domain.

In some embodiments, the first and/or the second MAM and the NTCRM are comprised of the transmembrane/membrane anchored domain, optional cytosolic domain, optional co-stimulatory domain, optional hinge domain and/or optional extracellular domain of a non-T cell receptor and/or a signaling adaptor.

In some embodiments, the first and/or the second MAM and the NTCRM are comprised of the transmembrane/membrane anchored domain, optional cytosolic domain, optional co-stimulatory domain, optional hinge domain and/or optional extracellular domain that are all derived from a single non-T cell receptor and/or a single signaling adaptor or variants thereof. In an exemplary embodiment, the first polypeptide chain comprises the hinge domain, transmembrane domain and cytosolic domain all derived from CD3 and the second polypeptide chain comprises the hinge domain, transmembrane domain and cytosolic domain all derived from FcRγ or CD16A.

In some embodiments, the first and/or the second MAM and the NTCRM are comprised of the transmembrane/membrane anchored domain, optional cytosolic domain, optional hinge domain and/or optional extracellular domain derived are derived from one or more different non-T cell receptor and/or a signaling adaptor or variants thereof. In an exemplary embodiment, the first polypeptide chain comprises a CD3 hinge domain, a CD3ƒ transmembrane domain and an FcRγ cytosolic domain and the second polypeptide chain comprises the DAP10 hinge domain, DAP 10 transmembrane domain attached to a cytosolic domain comprising a 41-BB costimulatory domain and a CD3ƒ activation domain.

In some embodiments, the two transmembrane/membrane anchored domains, optional cytosolic domains, optional co-stimulatory domain, optional hinge domains and/or optional extracellular domains are identical in sequence and are derived from the same protein. In some embodiments, the two transmembrane/membrane anchored domains, optional cytosolic domains, optional co-stimulatory domain, optional hinge domains and/or optional extracellular domains differ in sequence and/or are derived from different proteins.

In some embodiments, the two transmembrane/membrane anchored domains, optional cytosolic domains, optional co-stimulatory domain, optional hinge domains and/or optional extracellular domains are different in sequence and/or are derived from different proteins.

In one embodiment, the disclosure provides a double chain SAR that specifically bind to a target antigen comprising a) a first chain comprising one or more heterologous antigen binding domains that are operationally linked via one or more optional linkers to the extracellular domain of a signaling receptor or variant thereof; and b) a second chain comprising one or more heterologous antigen binding domains that are operationally linked via one or more optional linkers to the extracellular domain of a second signaling receptor or a variant thereof. In an embodiment, the one or both signaling receptors are naturally occurring. In an embodiment, the one or both signaling receptors are naturally occurring non-T cell receptors. In an embodiment, at least one heterologous antigen binding domain (e.g., vL, Vα, or Vγ etc.) present on the first chain associate with at least one heterologous antigen binding domain (e.g., vH, V3 or Vδ etc.) present on the second chain to form an antigen-binding module (e.g., Fv or TCR-Fv etc.) that specifically binds to a target antigen. In some embodiments, the target antigen is a cell surface antigen. In some embodiments, the target antigen is a complex comprising a peptide and an MHC protein (such as an MHC class I protein or an MHC class II protein). In some embodiments, the SAR binds to the target antigen with an equilibrium dissociation constant (Kd) from about 0.1 pM to about 500 nM. In some embodiments, the first polypeptide chain and the second polypeptide chain are linked via one or more disulfide bonds. In some embodiments, the first peptide linker and the second peptide linker are linked via one or more disulfide bonds. In some embodiments, there is a disulfide bond between a residue in the first optional linker in the first polypeptide chain and a residue in the second optional linker in the second polypeptide chain. In some embodiments, the first linker and/or the second linker are, individually, from about 5 to about 500 amino acids in length. In some embodiments, the first and/or second peptide linkers comprise, individually, a constant domain or fragment thereof from an immunoglobulin or T cell receptor subunit. In some embodiments, the first and/or second peptide linkers comprise, individually, a CH1, CH2, CH3, CH4 or CL antibody domain, or a fragment thereof. In some embodiments, the first and/or second peptide linkers comprise, individually, a Cα, C3, Cy, or Cδ TCR domain, or a variant or a fragment thereof. In some embodiments, the first and/or second linkers comprise, individually, an Ig like linker (e.g., IgCL, IgCHT etc.) derived from an immunoglobulin (e.g., SEQ ID NO: 3536-3551) or a TCR-Ig like linker (e.g., TCRb-Ig3, SEQ ID NO: 3560; TCRa-Ig3, SEQ ID NO:3562; TCRg-Ig3, SEQ ID NO: 3566; or TCRd-Ig3, SEQ ID NO: 3568 etc.) or a variant or a fragment thereof. In some embodiments, first and/or second peptide linkers comprise mutations that increase their expression, affinity and chain pairing. In some embodiments, the first and/or the second signaling receptor form a non-T cell receptor module (NTCRM) that is capable of recruiting at least one signaling adaptor (e.g., CD3ƒ or NCAM etc.). In some embodiments, the signaling adaptor is selected from the group consisting of CD3ƒ, FcRγ, DAP10 and/or DAP12.

In one embodiment, the disclosure provides a double chain SAR that specifically binds an antigen comprising a) a first antigen binding domain comprising vL (variable domain of light chain of an antibody), a Va (variable domain of TCRα or Vα), or a Vg (Variable domain of TCRg or Vγ) domain that is operationally linked via an optional linker to the entire or partial extracellular antigen binding domain of a non-TCR signaling receptor chain or a variant thereof, and b) second antigen binding domain a vH (variable domain of heavy chain of an antibody), a Vb (variable domain of TCRb or VD) or a Vd domain (variable domain of TCRd or Vδ) that is operationally linked via an optional linker to the entire or partial extracellular antigen binding domain of a second non-TCR signaling receptor chain or a variant thereof, wherein the vL, Vα or Vγ domains of the first antigen-binding domain and the vH, Vβ or Vδ domains of the second antigen-binding domain form a Fv or TCR-Fv like antigen-binding module that specifically binds to the target antigen. In one embodiment, the disclosure provides a double chain SAR that specifically binds an antigen comprising a) a vL domain that is operationally linked via an optional peptide linker to the entire or partial extracellular antigen binding domain of a non-TCR signaling receptor chain or a variant thereof, and b) a vH domain that is operationally linked via an optional peptide linker to the entire or partial extracellular antigen binding domain of a second non-TCR signaling receptor or a variant thereof, wherein the vL and vH domains form an Fv like antigen binding module that specifically binds to a target antigen in an MHC-dependent and/or MHC-independent manner. In one embodiment, the disclosure provides a double chain SAR that specifically binds an antigen comprising a) a Vα domain that is operationally linked via an optional peptide linker to the entire or partial extracellular antigen binding domain of a non-TCR signaling receptor or a variant thereof, and b) a Vβ domain that is operationally linked via an optional peptide linker to the entire or partial extracellular antigen binding domain of a second non-TCR signaling receptor or a variant thereof wherein the Vα and Vβ domains form an TCR-Fv like antigen binding module that specifically binds to a peptide/MHC complex in an MHC-dependent manner. In one embodiment, the disclosure provides a double chain SAR that specifically binds an antigen comprising a) a Vγ domain that is operationally linked via an optional peptide linker to the entire or partial extracellular antigen binding domain of a non-TCR signaling receptor or a variant thereof, and b) a Vδ domain that is operationally linked via an optional peptide linker to the entire or partial extracellular antigen binding domain of a second non-TCR signaling receptor or a variant thereof, wherein the Vγ and Vδ domains form an TCR-Fv like antigen binding module that specifically binds to an antigen in MHC-dependent or MHC-independent manner. In an exemplary embodiment, a non-TCR signaling receptor is selected from but not limited to one or more of the following: CD16A, CD16B, CD64, CD32, NKp30, NKp44, NKp46, KIR2DL1, KIR2DL2, KIR2DL3, KIR2DL5A, KIR2DL5B, KIR3DL1, KIR3DL2, KIR3DL4, KIR2DL4, KIR2DS1, KIR2DS2, KIR2DS3, KIR2DS4, KIR2DS5, KIR3DS1, DNAM-1, 2B4, OX40, CD28, 4-1BB, CD27, CD81, CD2, CD5, TNFR-I, TNFR-II, Fas, CD30, CD40, CRTAM, TIGIT, CD96, SLAMF6, SLAMF7, CD100, CD160, and ILT2. In some embodiments, the first and/or second peptide linkers comprise, individually, a constant domain or fragment thereof from an immunoglobulin or T cell receptor subunit. In some embodiments, the first and/or second peptide linkers comprise, individually, a CH1, CH2, CH3, CH4 or CL antibody domain, or a fragment thereof. In some embodiments, the first and/or second peptide linkers comprise, individually, a Cα, Cβ, Cγ, or Cδ TCR domain, or a variant or a fragment thereof. In some embodiments, the first and/or second linkers comprise, individually, an Ig like linker (e.g., IgCL, IgCH1 etc.) derived from an immunoglobulin (e.g., SEQ ID NO: 3536-3551) or a TCR-Ig like linker (e.g., TCRb-Ig3, SEQ ID NO: 3560; TCRa-Ig3, SEQ ID NO:3562; TCRg-Ig3, SEQ ID NO: 3566; or TCRd-Ig3, SEQ ID NO: 3568 etc.) or a variant or a fragment thereof. In some embodiments, first and/or second peptide linkers comprise mutations that increase their expression, affinity and chain pairing. In some embodiments, the first and the second polypeptide chains further comprise one or more autonomous antigen binding domains (AABD) that are attached to the N-terminus or near the N-terminus of the first (e.g., vL, Vα or Vγ) and/or the second (e.g., vH, V3 or Vδ) antigen binding domains.

In one embodiment, the disclosure provides a double chain SAR that specifically binds an antigen comprising a) a first antigen binding domain comprising vL (variable domain of light chain of an antibody), a Va (variable domain of TCRα or Vα), or a Vg (Variable domain of TCRg or Vγ) domain that is operationally linked via an optional peptide linker to the entire or partial hinge domain of a non-TCR signaling receptor chain or a variant thereof; and b) second antigen binding domain a vH (variable domain of heavy chain of an antibody), a Vb (variable domain of TCRb or VD) or a Vd domain (variable domain of TCRd or Vδ) that is operationally linked via an optional peptide linker to the entire or partial hinge domain of a second non-TCR signaling receptor chain or a variant thereof, wherein the vL, Vα or Vγ domains of the first antigen-binding domain and the vH, Vβ or Vδ domains of the second antigen-binding domain form a Fv or TCR-Fv like antigen-binding module that specifically binds to the target antigen. In one embodiment, the disclosure provides a double chain SAR that specifically binds an antigen comprising a) a vL domain that is operationally linked via an optional peptide linker to the entire or partial hinge domain of a non-TCR signaling receptor chain or a variant thereof, and b) a vH domain that is operationally linked via an optional peptide linker to the entire or partial hinge domain of a second non-TCR signaling receptor or a variant thereof, wherein the vL and vH domains form an Fv like antigen binding module that specifically binds to a target antigen in an MHC-dependent and/or MHC-independent manner. In one embodiment, the disclosure provides a double chain SAR that specifically binds an antigen comprising a) a Vα domain that is operationally linked via an optional peptide linker to the entire or partial hinge domain of a non-TCR signaling receptor or a variant thereof, and b) a Vβ domain that is operationally linked via an optional peptide linker to the entire or partial hinge domain of a second non-TCR signaling receptor or a variant thereof wherein the Vα and Vβ domains form an TCR-Fv like antigen binding module that specifically binds to a peptide/MHC complex in an MHC-dependent manner. In one embodiment, the disclosure provides a double chain SAR that specifically binds an antigen comprising a) a Vγ domain that is operationally linked via an optional peptide linker to the entire or partial hinge domain of a non-TCR signaling receptor or a variant thereof, and b) a Vδ domain that is operationally linked via an optional peptide linker to the entire or partial hinge domain of a second non-TCR signaling receptor or a variant thereof, wherein the Vγ and Vδ domains form an TCR-Fv like antigen binding module that specifically binds to an antigen in MHC-dependent or MHC-independent manner. In an exemplary embodiment, a non-TCR signaling receptor is selected from but not limited to one or more of the following: CD16A, CD16B, CD64, CD32, NKp30, NKp44, NKp46, KIR2DL1, KIR2DL2, KIR2DL3, KIR2DL5A, KIR2DL5B, KIR3DL1, KIR3DL2, KIR3DL4, KIR2DL4, KIR2DS1, KIR2DS2, KIR2DS3, KIR2DS4, KIR2DS5, KIR3DS1, DNAM-1, 2B4, OX40, CD28, 4-1BB, CD27, CD81, CD2, CD5, TNFR-I, TNFR-II, Fas, CD30, CD40, CRTAM, TIGIT, CD96, SLAMF6, SLAMF7, CD100, CD160, and ILT2. In some embodiments, the first and/or second peptide linkers comprise, individually, a constant domain or fragment thereof from an immunoglobulin or T cell receptor subunit. In some embodiments, the first and/or second peptide linkers comprise, individually, a CH1, CH2, CH3, CH4 or CL antibody domain, or a fragment thereof. In some embodiments, the first and/or second peptide linkers comprise, individually, a Cα, Cβ, Cγ, or Cδ TCR domain, or a variant or a fragment thereof. In some embodiments, the first and/or second peptide linkers comprise, individually, an Ig like linker (e.g., IgCL, IgCH1 etc.) derived from an immunoglobulin (e.g., SEQ ID NO: 3536-3551) or a TCR-Ig like linker (e.g., TCRb-Ig3, SEQ ID NO: 3560; TCRa-Ig3, SEQ ID NO:3562; TCRg-Ig3, SEQ ID NO: 3566; or TCRd-Ig3, SEQ ID NO: 3568 etc.) or a fragment thereof. In some embodiments, first and/or second peptide linkers comprise mutations that increase their expression, affinity and chain pairing. In some embodiments, the first and the second polypeptide chains further comprise one or more autonomous antigen binding domains (AABD) that are attached to the N-terminus or near the N-terminus of the first (e.g., vL, Vα or Vγ) and/or the second (e.g., vH, Vβ or Vδ) antigen binding domains.

In one embodiment, the disclosure provides a double chain SAR that specifically binds an antigen comprising a) a first antigen binding domain comprising vL (variable domain of light chain of an antibody), a Va (variable domain of TCRα or Vα), or a Vg (Variable domain of TCRg or Vγ) domain that is operationally linked via an optional peptide linker to the entire or partial transmembrane/membrane anchoring domain of a non-TCR signaling receptor chain or a variant thereof; and b) second antigen binding domain a vH (variable domain of heavy chain of an antibody), a Vb (variable domain of TCRb or VD) or a Vd domain (variable domain of TCRd or Vδ) that is operationally linked via an optional peptide linker to the entire or partial transmembrane/membrane anchoring domain of a second non-TCR signaling receptor chain or a variant thereof, wherein the vL, Vα or Vγ domains of the first antigen-binding domain and the vH, Vβ or Vδ domains of the second antigen-binding domain form a Fv or TCR-Fv like antigen-binding module that specifically binds to the target antigen. In one embodiment, the disclosure provides a double chain SAR that specifically binds an antigen comprising a) a vL domain that is operationally linked via an optional peptide linker to the entire or partial transmembrane/membrane anchoring domain of a non-TCR signaling receptor chain or a variant thereof; and b) a vH domain that is operationally linked via an optional peptide linker to the entire or partial transmembrane/membrane anchoring domain of a second non-TCR signaling receptor or a variant thereof, wherein the vL and vH domains form an Fv like antigen binding module that specifically binds to a target antigen in an MHC-dependent and/or MHC-independent manner. In one embodiment, the disclosure provides a double chain SAR that specifically binds an antigen comprising a) a Va domain that is operationally linked via an optional peptide linker to the entire or partial transmembrane/membrane anchoring domain of a non-TCR signaling receptor or a variant thereof; and b) a Vβ domain that is operationally linked via an optional peptide linker to the entire or partial transmembrane/membrane anchoring domain of a second non-TCR signaling receptor or a variant thereof wherein the Vα and Vβ domains form an TCR-Fv like antigen binding module that specifically binds to a peptide/MHC complex in an MHC-dependent manner. In one embodiment, the disclosure provides a double chain SAR that specifically binds an antigen comprising a) a Vγ domain that is operationally linked via an optional peptide linker to the entire or partial transmembrane/membrane anchoring domain of a non-TCR signaling receptor or a variant thereof; and b) a Vδ domain that is operationally linked via an optional peptide linker to the entire or partial transmembrane/membrane anchoring domain of a second non-TCR signaling receptor or a variant thereof, wherein the Vγ and Vδ domains form an TCR-Fv like antigen binding module that specifically binds to an antigen in MHC-dependent or MHC-independent manner. In an exemplary embodiment, a non-TCR signaling receptor is selected from but not limited to one or more of the following: CD16A, CD16B, CD64, CD32, NKp30, NKp44, NKp46, KIR2DL1, KIR2DL2, KIR2DL3, KIR2DL5A, KIR2DL5B, KIR3DL1, KIR3DL2, KIR3DL4, KIR2DL4, KIR2DS1, KIR2DS2, KIR2DS3, KIR2DS4, KIR2DS5, KIR3DS1, DNAM-1, 2B4, OX40, CD28, 4-1BB, CD27, CD81, CD2, CD5, TNFR-I, TNFR-II, Fas, CD30, CD40, CRTAM, TIGIT, CD96, SLAMF6, SLAMF7, CD100, CD160, and ILT2. In some embodiments, the first and/or second peptide linkers comprise, individually, a constant domain or fragment thereof from an immunoglobulin or T cell receptor subunit. In some embodiments, the first and/or second peptide linkers comprise, individually, a CH1, CH2, CH3, CH4 or CL antibody domain, or a fragment thereof. In some embodiments, the first and/or second peptide linkers comprise, individually, a Cα, Cβ, Cγ, or Cδ TCR domain, or a variant or a fragment thereof. In some embodiments, the first and/or second peptide linkers comprise, individually, an Ig like linker (e.g., IgCL, IgCHT etc.) derived from an immunoglobulin (e.g., SEQ ID NO: 3536-3551) or a TCR-Ig like linker (e.g., TCRb-Ig3, SEQ ID NO: 3560; TCRa-Ig3, SEQ ID NO:3562; TCRg-Ig3, SEQ ID NO: 3566; or TCRd-Ig3, SEQ ID NO: 3568 etc.) or a fragment thereof. In some embodiments, first and/or second peptide linkers comprise mutations that increase their expression, affinity and chain pairing. In some embodiments, the first and the second polypeptide chains further comprise one or more autonomous antigen binding domains (AABD) that are attached to the N-terminus or near the N-terminus of the first (e.g., vL, Vα or Vγ) and/or the second (e.g., vH, Vβ or Vδ) antigen binding domains.

In one embodiment, the disclosure provides a double chain SAR that specifically binds an antigen comprising a) a first antigen binding domain that is operationally linked via an optional peptide linker to the entire or partial transmembrane/membrane anchoring domain of a non-TCR signaling receptor chain or a signaling adaptor or a variant or a fragment thereof; and b) second antigen binding domain that is operationally linked via an optional peptide linker to the entire or partial transmembrane/membrane anchoring domain of a second non-TCR signaling receptor chain or a signaling adaptor or a variant or a fragment thereof. In some embodiments, the first and/or second peptide linkers comprise, individually, a constant domain or fragment thereof from an immunoglobulin or T cell receptor subunit. In some embodiments, the first and/or second peptide linkers comprise, individually, a CH1, CH2, CH3, CH4 or CL antibody domain, or a fragment thereof. In some embodiments, the first and/or second peptide linkers comprise, individually, a Cα, Cβ, Cγ, or Cδ TCR domain, or a variant or a fragment thereof. In an embodiment, the first antigen-binding domain and the second antigen-binding domain specifically binds to their respective target antigens. In an embodiment, the one or both of the antigen binding domains are autonomous antigen binding domains (e.g., AABD, e.g., vHH, FHVH, SVH, centyrin, DARPIN, svd-TCR, adaptor, adaptor binding domain, ligand binding domain of a receptor, receptor binding domain of a ligand etc.). In an embodiment, the one or both of the antigen binding domains comprise an antibody, an antibody fragment (e.g., Fab), scFv, a TCR or a scTCR. In some embodiments, the first and the second polypeptide chains further comprise one or more autonomous antigen binding domains (AABD) that are attached to the N-terminus or near the N-terminus of the first and/or the second antigen binding domains. In an embodiment, the first and/or the second signaling chain comprise a cytosolic domain comprising an activation domain and an optional costimulatory domain.

In an embodiment, the double chain SAR retains the entire or partial binding properties of the original signaling receptors (e.g., non-T cell receptors) and also acquires the binding properties conferred by the one or more heterologous antigen binding domains. In an embodiment, the double chain SAR retains the entire or partial binding properties of the original signaling receptors. In an embodiment, the double chain SAR additionally acquires the binding properties conferred by the one or more heterologous antigen binding domains. In an embodiment, the double chain SAR retains the signaling properties of the two signaling receptors. In an embodiment, the double chain SAR acquires new signaling properties that are not exhibited by either of the two signaling receptors when activated alone. In an embodiment, the double chain SAR acquires new signaling properties that are additive of the signaling properties of the two signaling receptors when activated alone. In an embodiment, the double chain SAR acquires new signaling properties that are synergistic of the signaling properties of the two signaling receptors when activated alone.

In an embodiment, the signaling receptor comprising one or both chains of a double chain SAR is a Type I membrane protein with a single transmembrane domain. In an embodiment, the signaling receptor is a naturally occurring signaling receptor. In an embodiment, the signaling receptor is a non-TCR signaling receptor. In an embodiment, one or both chains of the double chain SAR are capable of recruiting a signaling adaptor (e.g., CD3ƒ, FcRγ, DAP10 or DAP12 etc.). In an embodiment, one or both chains of the double chain SAR are capable of recruiting a signaling adaptor that comprises an activation domain. In an embodiment, one or both chains of the double chain SAR are capable of recruiting a signaling adaptor that comprises one or more ITAMs. In an embodiment, one or both chains of the double chain SAR are capable of recruiting a signaling adaptor that comprises one or more ITIMs. In an embodiment, one or both chains of the double chain SAR are capable of recruiting a signaling adaptor that comprise a costimulatory domain. In an embodiment the signaling adaptor is naturally occurring. In an embodiment, the signaling adaptor is non-naturally occurring. In an embodiment, one or both chains of the double chain SAR are capable of recruiting a signaling adaptor that activates intracellular signaling pathways (e.g., NFAT, NF-κB, ERK, PI3K etc.). In an embodiment, one or both chains of the double chain SAR are capable of recruiting a signaling adaptor that inhibits intracellular signaling pathways (e.g., NFAT, NF-κB, ERK, PI3K etc.).

In an embodiment, one or both chains of the double chain SAR comprise a costimulatory domain. In an embodiment, one or both chains of the double chain SAR comprise an activation domain and a costimulatory domain. In an embodiment, one or both chains of the double chain SAR comprise an intracellular activation domain. In an exemplary embodiment, one or both chains of the double chain SAR comprise an intracellular activation domain derived from a signaling adaptor. In exemplary embodiments, one or both chains of a double chain SAR comprise an intracellular activation domain derived from CD3ƒ, FcRγ, DAP10 or DAP12. In an embodiment, the activation domain present in one or both chains of a double chain SAR comprises one or more ITAMs. In an embodiment, one or both chains of the double chain SAR comprise an activation domain that contains one or more ITAMs. In an embodiment, one or both chains of the double chain SAR comprise an activation domain that contain two or more ITAM motifs. In an embodiment, one or both chains of a double chain SAR comprise an activation domain that contains a single ITAM. In an embodiment, one or both chains of a double chain SAR lack an ITAM. In an embodiment, one or both chains of a double chains SAR comprise an activation domain that contains a tyrosine-based motif (YINM). In an embodiment, one or both chains of a double chains SAR comprise an activation domain that recruits the p85 subunit of PI3K and/or Grb2. In an embodiment, one or both chains of a double chains SAR comprise an activation domain that activate one or more of NFAT, PI3K, NF—KB and/or ERK signaling pathways.

In an embodiment, the SAR comprises an intracellular inhibitory domain. In an exemplary embodiment, the SAR comprises an intracellular inhibitory domain derived from PD1. In an embodiment, the inhibitory domain of SAR comprises one or more ITIM motifs.

In an embodiment, the SAR is capable of recruiting signaling adaptors. In an exemplary embodiment, the SAR is capable of recruiting one or more signaling adaptors selected from the group of, but not limited to, CD3ƒ, FcRγ, DAP10 and DAP12.

In one embodiment, the disclosure provides a double chain SAR comprising one or more heterologous antigen binding domains that are operationally linked via optional linkers to two polypeptide chains at least one of which can recruit a signaling adaptor. In one embodiment, the disclosure provides a double chain SAR comprising one or more heterologous antigen binding domains that are operationally linked via optional linkers to two polypeptide chains each of which can recruit a signaling adaptor. In an embodiment, one or both polypeptides comprise a hinge domain, a transmembrane domain and a cytosolic domain. In an embodiment, at least one polypeptide comprises a transmembrane domain. In an embodiment, both polypeptides comprise a transmembrane domain. In an embodiment, at least one polypeptide comprises a cytosolic domain. In an embodiment, both polypeptides comprise a cytosolic domain. In one embodiment, the disclosure provides a double chain SAR comprising one or more heterologous antigen binding domains that are operationally linked via optional linkers to two polypeptide chains at least one of which can recruit a signaling adaptor. In one embodiment, the disclosure provides a double chain SAR comprising one or more heterologous antigen binding domains that are operationally linked via optional linkers to two polypeptide chains comprising one or more intracellular activation domains. In one embodiment, the disclosure provides a double chain SAR comprising one or more heterologous antigen binding domains that are operationally linked via optional linkers to two polypeptide chains at least one of which comprises an intracellular activation domain.

In an embodiment, the signaling adaptor is a non-TCR/CD3 signaling adaptor. In an embodiment, the signaling adaptor is not a component of the TCR/CD3 signaling complex. In an embodiment, the signaling adaptor is not CD3ƒ. In an embodiment, the signaling adaptor is a non-natural signaling adaptor; i.e., a signaling adaptor that does not exist in nature. In an embodiment, the signaling adaptor comprise one or more ITAMs. In an embodiment, the signaling adaptor comprises one or more ITIMs. In an embodiment, the signaling adaptor is a disulfide linked dimeric protein. In an embodiment, the signaling adaptor is a type I transmembrane protein. In an embodiment, the signaling adaptor is capable of recruiting signaling proteins, e.g., protein kinases, e.g., ZAP70. In an embodiment, the signaling adaptor is capable of activating one or more cellular signaling pathways, e.g., NFAT, NF-κB, ERK, PI3K etc.

In an embodiment, the two non-TCR signaling receptor chains comprising the two chains of a double chain SAR are of the same type and sequence (e.g., both signaling chains comprise the extracellular, transmembrane and cytosolic domains of CD16A, NKp46 or NKp30 etc.). Exemplary such double chain SAR are represented by SEQ ID NO: 6383-6293, 4675, 4696 and 8344) In an embodiment, the two signaling chains of a double chain SAR are of the different type (e.g., one chain comprises the extracellular, transmembrane and cytosolic domains of CD16A and the second chain comprises the extracellular, transmembrane and cytosolic domains of NKp30 or comprises the hinge, transmembrane and cytosolic domains of CD16A and the second chain comprises the hinge, transmembrane and cytosolic domains of CD3ƒetc.). Exemplary such SARs are represented by SEQ ID NO: 4695 and 4670.

In an embodiment, the two signaling chains of a double chain SAR are derived from the same receptor (e.g., both chains are derived from CD16A). An exemplary such SAR is represented by SEQ ID NO: 4676. In an embodiment, the two signaling chains of a double chain SAR are derived from different receptors (e.g., one chain is derived from NKp44 and the second chain is derived from NKp30 etc.). An exemplary such SAR is represented by SEQ ID NO: 4713.

In an embodiment, the disclosure provides a double chain SAR comprising a first chain that is derived from a non-TCR receptor signaling chain (e.g., CD16A) and a second chain that is derived from a signaling adaptor (e.g., CD3ƒ or FcRy). An exemplary such a SAR is represented by SEQ ID NO: 4670.

In an embodiment, a double chain SAR may comprise first chain that is derived from a non-TCR receptor signaling chain (e.g., CD16A) and a second chain that is derived from a TCR signaling chain (e.g., constant chain of TCRα, TCRβ, TCRγ, TCRδ, preTCRα or a variant or a fragment thereof). In some embodiments, the disclosure provides a double chain SAR comprising one non-TCR module (or NTCRM) and one TCR module (or TCRM). Exemplary such SARs are represented by SEQ ID NO: 4708-4710.

In an embodiment, the optional linker between the vL/vH, Vα/Vβ, Vγ/Vδ chains of the heterologous antigen binding domain and the non-TCR signaling chains is an Ig like linker (SEQ ID NO (DNA): 1142-1175 and SEQ ID NO (PRT): 3536-3569) represented in Table 13.

In one embodiment, the signaling receptor that is used in the construction of a double chain SAR is any receptor expressed on the surface of an immune cell. In one embodiment, the signaling receptor is a signaling chain of a naturally occurring signaling receptor which is expressed on the surface of an immune cell. In an exemplary embodiment, the immune cell is selected from but not limited to a T cell, an NK cell, a monocyte/macrophage, a granulocyte and a B cell. Exemplary signaling receptors that can be used in the construction of a double chain SAR of the disclosure include CD16A, CD16B, CD64, CD32, NKp30, NKp44, NKp46, KIR2DL1, KIR2DL2, KIR2DL3, KIR2DL5A, KIR2DL5B, KIR3DL1, KIR3DL2, KIR3DL4, KIR2DL4, KIR2DS1, KIR2DS2, KIR2DS3, KIR2DS4, KIR2DS5, KIR3DS1, NKG2D, NKG2C, NKG2A, NKG2E, NKG2F, DNAM-1, 2B4, OX40, CD28, 4-1BB, CD27, CD81, CD2, CD5, TNFR-I, TNFR-II, Fas, CD30, CD40, CRTAM, TIGIT, CD96, SLAMF6, SLAMF7, CD100, CD160, CEACAM, ILT2, KLRG1, LAIR1, CD161, Siglec3, Siglec-7 and Siglec-9 etc. or variants thereof. In further embodiment, an autonomous antigen binding domain (e.g., fully human vH domain, vHH, single chain TCR etc.), non-immunoglobulin antigen binding domain (e.g., Centyrin, affibody etc.), ligand (e.g., APRIL, TPO, NKG2D-YA-G4Sx3-NKG2D-YA etc.), and extracellular domain of a receptor (e.g., NKp30, NKp44, NKp46, NKG2D, CD16A etc.), an adaptor binding domain (e.g., EZip, RZip, E4, R4 etc.) can be operationally linked the amino-terminus or near the amino-terminus of the vL, vH, Va, VD, Vγ or Vδ chains of the SAR to confer additional antigen binding capabilities on the SAR.

In some embodiments, there is provided a SAR (such as an isolated SAR) that specifically binds to a target antigen, wherein the SAR comprises: a) a first polypeptide chain comprising a first antigen-binding domain comprising a vL, a Va or a Vγ domains and a first Membrane associated module (MAM); and b) a second polypeptide chain comprising a second antigen-binding domain comprising a vH, a Vβ or a Vδ domains and a second Membrane associated module (MAM), wherein the vL, Vα or Vγ domains of the first antigen-binding domain and the complementary vH, Vβ or Vδ domains of the second antigen-binding domain form a Fv or TCR-Fv like antigen-binding module that specifically binds to the target antigen, and wherein the first MAM and/or the second MAM form a non-T cell receptor module (NTCRM). In an embodiment, the SAR is capable of activating at least one signaling pathway and/or recruiting at least one signaling adaptor. In an embodiment, the first and/or second MAM are derived from, but not limited to, one or more of the following signaling adaptors: CD3ƒ, FcRγ. DAP10 or DAP12 or a variant or fragment thereof. In an embodiment, a NTCRM is comprised of, but not limited to, one or more of the following signaling adaptors: CD3ƒ, FcRγ. DAP10 or DAP12. In some embodiments, the signaling adaptor is a non-CD3 adaptor (NCAM). In some embodiments, the signaling adaptor is not CD3ƒ. In some embodiments, the first polypeptide chain and the second polypeptide chain are linked via one or more disulfide bonds. In some embodiments, the first polypeptide chain further comprises a first peptide linker between the first antigen-binding domain and the first MAM. In some embodiments, the second polypeptide chain further comprises a second peptide linker between the second antigen-binding domain and the second MAM. In some embodiments, the first polypeptide chain and the second polypeptide chain are linked via one or more disulfide bonds. In some embodiments, the first peptide linker and the second peptide linker are linked via one or more disulfide bonds. In some embodiments, the first peptide linker and/or the second peptide linker are, individually, from about 5 to about 500 amino acids in length. In some embodiments, the first and/or second peptide linkers comprise, individually, a constant domain or fragment thereof from an immunoglobulin or T cell receptor subunit. In some embodiments, the first and/or second peptide linkers comprise, individually, a CH1, CH2, CH3, CH4 or CL antibody domain, or a fragment thereof. In some embodiments, the first and/or second peptide linkers comprise, individually, a Cα, Cβ, Cγ, or Cδ TCR domain, or a variant or a fragment thereof. In some embodiments, first and/or second peptide linkers comprise mutations that increase their affinity and chain pairing. In some embodiments, the first and/or second linkers comprise, individually, an Ig like linker (e.g., IgCL, IgCH1 etc.) derived from an immunoglobulin (e.g., SEQ ID NO: 3536-3551) or a TCR-Ig like linker (e.g., TCRb-Ig3, SEQ ID NO: 3560; TCRa-Ig3, SEQ ID NO:3562; TCRg-Ig3, SEQ ID NO: 3566; or TCRd-Ig3, SEQ ID NO: 3568 etc.) or a fragment thereof. In some embodiments, first and/or second peptide linkers comprise mutations that increase their expression, affinity and chain pairing. In an embodiment, the first and the second antigen binding domains comprise complementary chains (e.g., vL and vH, Vα and Vβ or Vγ and Vδ). In some embodiments, the first and the second polypeptide chains further comprise one or more autonomous antigen binding domains (AABD) that are attached to the N-terminus or near the N-terminus of the first (e.g., vL, Vα or Vγ) and/or the second (e.g., vH, Vβ or Vδ) antigen binding domains. In some embodiments, the SAR binds to the target antigen with an equilibrium dissociation constant (Kd) from about 0.1 pM to about 500 nM. In some embodiments, the target antigen is a cell surface antigen. In an exemplary embodiment, the target antigen is one or more of the antigens listed in Table B. In some embodiments, the cell surface antigen is selected from the group consisting of protein, carbohydrate, and lipid. In some embodiments, the cell surface antigen is one or more of CD19, CD20, CD22, CD33, CD70, CD123, CD138, CLL-1, FLT3, Claudin 18.2, BCMA, GCC, MPL, SLAMF7, RORI, ROR2, GPRC5D, FCRL5, MSLN, EGFR, EGFRviii, PSMA, PSCA, KLK2, IL13Ra2, TROP2, PTK7, DLL3, Muc1, Muc16 or Her2. In some embodiments, the target antigen is a complex comprising a peptide and a major histocompatibility complex (MHC) protein. In exemplary embodiments, the peptide/MHC complex comprises a peptide derived from one or more of NY-ESO-1, MAGE-A2, MAGE-A3, MAGE4, WT1, AFP, TERT, MART-1, pp66-CMV, HPV16-E7, PRAME, EBV-LMP2A, HIV-1, PSA or gp100.

In some embodiments, according to any of the SARs (such as isolated SARs) described above, the first MAM further comprises a first hinge domain or fragment thereof N-terminal to the first transmembrane domain, the second MAM further comprises a second hinge domain or fragment thereof N-terminal to the second transmembrane domain. In some embodiments, the NTCRM comprises a disulfide bond between a residue in the first hinge domain and a residue in the second hinge domain. In some embodiments, the first MAM further comprises a first cytosolic domain C-terminal to the first transmembrane domain. In some embodiments, the second MAM further comprises a second cytosolic domain C-terminal to the second transmembrane domain. In an embodiment, the first and/or second cytosolic domains are activation domains comprising one or more ITAMs. In some embodiments, the SAR binds to the target antigen with an equilibrium dissociation constant (Kd) from about 0.1 pM to about 500 nM.

In some embodiments, according to any of the SARs (such as isolated SARs) described above, the first polypeptide chain further comprises a first co-stimulatory domain C-terminal to the first transmembrane domain. In some embodiments, the second polypeptide chain further comprises a second co-stimulatory domain C-terminal to the second transmembrane domain. In some embodiments, the first polypeptide chain further comprises a first signaling peptide N-terminal to the first antigen-binding domain. In some embodiments, the second polypeptide chain further comprises a second signaling peptide N-terminal to the second antigen-binding domain.

In one embodiment, the disclosure provides a double chain SAR that specifically binds an antigen comprising a) a first antigen binding domain comprising vL (variable domain of light chain of an antibody), a Va (variable domain of TCRα or Vα), or a Vg (Variable domain of TCRg or Vγ) domain that is operationally linked via an optional peptide linker to the entire or partial hinge domain of a signaling adaptor or a variant thereof; and b) second antigen binding domain a vH (variable domain of heavy chain of an antibody), a Vb (variable domain of TCRb or Vβ) or a Vd domain (variable domain of TCRd or Vδ) that is operationally linked via an optional peptide linker to the entire or partial hinge domain of a second signaling adaptor or a variant thereof, wherein the vL, Vα or Vγ domain of the first antigen-binding domain and the complementary vH, Vβ or Vδ domain of the second antigen-binding domain form a Fv or TCR-Fv like antigen-binding module that specifically binds to a target antigen. In one embodiment, the disclosure provides a double chain SAR that specifically binds an antigen comprising a) a vL domain that is operationally linked via an optional peptide linker to the entire or partial hinge domain of a signaling adaptor or a variant thereof; and b) a vH domain that is operationally linked via an optional peptide linker to the entire or partial hinge domain of a second signaling adaptor or a variant thereof, wherein the vL and vH domains form an Fv like antigen binding module that specifically binds to a target antigen in an MHC-dependent and/or MHC-independent manner. In one embodiment, the disclosure provides a double chain SAR that specifically binds an antigen comprising a) a Va domain that is operationally linked via an optional peptide linker to the entire or partial hinge domain of a signaling adaptor or a variant thereof; and b) a Vβ domain that is operationally linked via an optional peptide linker to the entire or partial hinge domain of a second signaling adaptor or a variant thereof wherein the Vα and Vβ domains form an TCR-Fv like antigen binding module that specifically binds to a peptide/MHC complex in an MHC-dependent manner. In one embodiment, the disclosure provides a double chain SAR that specifically binds an antigen comprising a) a Vγ domain that is operationally linked via an optional peptide linker to the entire or partial hinge domain of a signaling adaptor or a variant thereof; and b) a Vδ domain that is operationally linked via an optional peptide linker to the entire or partial hinge domain of a second signaling adaptor or a variant thereof, wherein the Vγ and Vδ domains form an TCR-Fv like antigen binding module that specifically binds to an antigen in MHC-dependent or MHC-independent manner. In an exemplary embodiment, a signaling adaptor is selected from but not limited to one or more of the following: CD3ƒ, FcRγ, DAP10 or DAP10 or a variant or a fragment thereof. In some embodiments, the signaling adaptor is a non-CD3 adaptor (NCAM). In some embodiments, the signaling adaptor is not CD3ƒ. In some embodiments, the first and/or second peptide linkers comprise, individually, a constant domain or fragment thereof from an immunoglobulin or T cell receptor subunit. In some embodiments, the first and/or second peptide linkers comprise, individually, a CH1, CH2, CH3, CH4 or CL antibody domain, or a fragment thereof. In some embodiments, the first and/or second peptide linkers comprise, individually, a Cα, Cβ, Cγ, or Cδ TCR domain, or a variant or a fragment thereof. In some embodiments, the first and/or second linkers comprise, individually, an Ig like linker (e.g., IgCL, IgCHT etc.) derived from an immunoglobulin (e.g., SEQ ID NO: 3536-3551) or a TCR-Ig like linker (e.g., TCRb-Ig3, SEQ ID NO: 3560; TCRa-Ig3, SEQ ID NO:3562; TCRg-Ig3, SEQ ID NO: 3566; or TCRd-Ig3, SEQ ID NO: 3568 etc.) or a variant or a fragment thereof. In some embodiments, first and/or second peptide linkers comprise mutations that increase their expression, affinity and chain pairing. In some embodiments, the signaling adaptor further comprises one or more co-stimulatory domains. In exemplary embodiments, a signaling adaptor comprises a co-stimulatory domain from CD28, 4-1BB, OX40, 2B4, CD27, CD81, CD2, CD5, BAFF-R, CD30, CD40, HVEM or ICOS, or a variant or a fragment thereof. In some embodiments, the first and/or second peptide linkers comprise, individually, an Ig like linker (e.g., IgCL, IgCH1 etc.) derived from an immunoglobulin (e.g., SEQ ID NO: 3536-3551) or a TCR-Ig like linker (e.g., TCRb-Ig3, SEQ ID NO: 3560; TCRa-Ig3, SEQ ID NO:3562; TCRg-Ig3, SEQ ID NO: 3566; or TCRd-Ig3, SEQ ID NO: 3568 etc.) or a fragment thereof. In some embodiments, first and/or second peptide linkers comprise mutations that increase their expression, affinity and chain pairing. In some embodiments, the first and the second polypeptide chains further comprise one or more autonomous antigen binding domains (AABD) that are attached to the N-terminus or near the N-terminus of the first (e.g., vL, Vα or Vγ) and/or the second (e.g., vH, Vβ or Vδ) antigen binding domains.

In one embodiment, the disclosure provides a double chain SAR that specifically binds an antigen comprising a) a first antigen binding domain comprising vL, a Vα, or Vγ domain that is operationally linked via an optional peptide linker to the entire or partial transmembrane/membrane anchoring domain of a signaling adaptor or a variant thereof; and b) second antigen binding domain comprising a vH, a Vβ or a Vδ domain that is operationally linked via an optional peptide linker to the entire or partial transmembrane/membrane anchoring domain of a second signaling adaptor or a variant thereof, wherein the vL, Vα or Vγ domain of the first antigen-binding domain and the complementary vH, Vβ or Vδ domain of the second antigen-binding domain form a Fv or TCR-Fv like antigen-binding module that specifically binds to the target antigen. In one embodiment, the disclosure provides a double chain SAR that specifically binds an antigen comprising a) a vL domain that is operationally linked via an optional peptide linker to the entire or partial transmembrane/membrane anchoring domain of a signaling adaptor or a variant thereof; and b) a vH domain that is operationally linked via an optional peptide linker to the entire or partial transmembrane/membrane anchoring domain of a second signaling adaptor or a variant thereof, wherein the vL and vH domains form an Fv like antigen binding module that specifically binds to a target antigen in an MHC-dependent and/or MHC-independent manner. In one embodiment, the disclosure provides a double chain SAR that specifically binds an antigen comprising a) a Va domain that is operationally linked via an optional peptide linker to the entire or partial transmembrane/membrane anchoring domain of a signaling adaptor or a variant thereof; and b) a Vβ domain that is operationally linked via an optional peptide linker to the entire or partial transmembrane/membrane anchoring domain of a second signaling adaptor or a variant thereof wherein the Va and Vβ domains form an TCR-Fv like antigen binding module that specifically binds to a peptide/MHC complex in an MHC-dependent manner. In one embodiment, the disclosure provides a double chain SAR that specifically binds an antigen comprising a) a Vγ domain that is operationally linked via an optional peptide linker to the entire or partial transmembrane/membrane anchoring domain of a signaling adaptor or a variant thereof; and b) a Vδ domain that is operationally linked via an optional peptide linker to the entire or partial transmembrane/membrane anchoring domain of a second signaling adaptor or a variant thereof, wherein the Vγ and Vδ domains form an TCR-Fv like antigen binding module that specifically binds to an antigen in MHC-dependent or MHC-independent manner. In an exemplary embodiment, a signaling adaptor is selected from but not limited to one or more of the following: CD3ƒ, FcRγ, DAP10 or DAP12 or a variant or a fragment thereof. In some embodiments, the signaling adaptor is a non-CD3 adaptor (NCAM). In some embodiments, the signaling adaptor is not CD3ƒ. In some embodiments, the signaling adaptor further comprises one or more co-stimulatory domains. In exemplary embodiments, a signaling adaptor comprises a co-stimulatory domain from CD28, 4-1BB, OX40, 2B4, CD27, CD81, CD2, CD5, BAFF-R, CD30, CD40, HVEM or ICOS, or a variant or a fragment thereof. In some embodiments, the first and/or second peptide linkers comprise, individually, a constant domain or fragment thereof from an immunoglobulin or T cell receptor subunit. In some embodiments, the first and/or second peptide linkers comprise, individually, a CH1, CH2, CH3, CH4 or CL antibody domain, or a fragment thereof. In some embodiments, the first and/or second peptide linkers comprise, individually, a Cα, Cβ, Cγ, or Cδ TCR domain, or a variant or a fragment thereof. In some embodiments, the first and/or second peptide linkers comprise, individually, an Ig like linker (e.g., IgCL, IgCHT etc.) derived from an immunoglobulin (e.g., SEQ ID NO: 3536-3551) or a TCR-Ig like linker (e.g., TCRb-Ig3, SEQ ID NO: 3560; TCRa-Ig3, SEQ ID NO:3562; TCRg-Ig3, SEQ ID NO: 3566; or TCRd-Ig3, SEQ ID NO: 3568 etc.) or a variant or a fragment thereof. In some embodiments, the first and the second polypeptide chains further comprise one or more autonomous antigen binding domains (AABD) that are attached to the N-terminus or near the N-terminus of the first (e.g., vL, Vƒ or Vγ) and/or the second (e.g., vH, V3 or Vδ) antigen binding domains.

In one embodiment, the disclosure provides a SAR comprising a) one or more heterologous antigen binding domains that are operationally linked via an optional linker to the amino-terminus or near the amino-terminus of one chain of a signaling adaptor (or a signaling chain) or a variant thereof; and b) one or more heterologous antigen binding domains that are operationally linked via an optional linker to the amino-terminus or near the amino-terminus of second chain of a signaling adaptor (or a signaling chain) or a variant thereof. In an embodiment, such a SAR retains the signaling capability of the original signaling adaptors (or signaling chains). In an embodiment, the SAR also acquires the binding capabilities conferred by the heterologous antigen binding domains. In an exemplary embodiment, the signaling adaptor is any signaling adaptor (or signaling chain) that can be expressed on the plasma membrane of a cell (e.g., an immune cell, e.g., an immune effector cell). In an exemplary embodiment, the immune cell is selected from but is not limited to a T cell, an NK cell, a monocyte/macrophage, a granulocyte or a B cell. Exemplary signaling adaptors (or signaling chains) that can be used for the construction of SAR of the disclosure include but are not limited to CD3z (CD3ƒ, FcRγ (FcεRIγ), DAP10 and DAP12 etc. or variants thereof.

The disclosure provides SARs in which one or more heterologous antigen binding domains are operationally linked to the extracellular domains (e.g., hinge or spacer domains) of one or more chains of a signaling adaptor. In an embodiment, the SAR comprises a signaling adaptor that is a component of a TCR complex (e.g., CD3ƒ, CD3R, CD3γ, CD3E etc.). In an embodiment, the SAR comprises a signaling adaptor (e.g., CD3ƒ) that interacts with TCRα, J, y and/or 6 chains of the TCR complex. In an embodiment, the SAR comprises a signaling adaptor that does not interacts with TCRα, J, y and/or 6 chains of the TCR complex. In an embodiment, the SAR comprises a signaling adaptor that has a conserved aspartic acid residue in its transmembrane domain which interacts with positive charged residues (lysine or arginine) in the transmembrane regions or TCRα, TCRβ, TCRγ or TCRδ. In an embodiment, the SAR comprises a signaling adaptor that lacks a conserved aspartic acid residue in its transmembrane domain. In an embodiment, the SAR comprises a signaling adaptor that is not a component of a TCR complex. In an embodiment, the signaling adaptor is a non-CD3 signaling adaptor (NCAM). In an embodiment, the signaling adaptor is not CD3ƒ or a variant thereof.

In some embodiments, according to any of the SARs (such as isolated SARs) described above, the SAR comprises a signaling adaptor (e.g., CD3) that activates cell signaling. In an embodiment, the SAR comprises a signaling adaptor that inhibits cell signaling. In an embodiment, the SAR comprises a signaling adaptor (e.g. CD3) that possesses one or more ITAM motifs. In an embodiment, the SAR comprises a signaling adaptor that possesses two or more ITAM motifs. In an embodiment, the SAR comprises a signaling adaptor (e.g., FcRy) that possesses a single ITAM motif. In an embodiment, the SAR comprises a signaling adaptors that lacks an ITAM motifs. In an embodiment, the SAR comprises a signaling adaptor (e.g., DAP10) that comprises a tyrosine-based motif (YINM). In an embodiment, the SAR comprises a signaling adaptor (e.g., DAP10) that recruits the p85 subunit of PI3K and/or Grb2. In an embodiment, the SAR comprises a signaling adaptor that is a disulfide linker dimer in its native form. In an embodiment, the signaling adaptor is not a disulfide linker dimer in its native form. In an embodiment, the SAR comprises a signaling adaptor (e.g., CD3ƒ) that in its native state contains an interchain disulfide bond located in its transmembrane region. In an embodiment, the SAR comprises a signaling adaptor (e.g., DAP10 and DAP12) which in its native state contains an interchain disulfide bond that is not located in its transmembrane region. In an embodiment, the SAR comprises a signaling adaptor that in its native state contains an interchain disulfide bond that is located in its extracellular region.

In some embodiments, according to any of the SARs (such as isolated SARs) described above, the extracellular domain of the signaling adaptor is less than 10 amino acids in length. In an embodiment, the extracellular domain of the signaling adaptor is less than 8 amino acids in length. In an embodiment, the extracellular domain of the signaling adaptor is more than 10 amino acids in length. In an embodiment, the extracellular domain of the signaling adaptor is more than 15 amino acids in length.

In some embodiments, according to any of the SARs (such as isolated SARs) described above, the SAR comprises a signaling adaptor that induces protein phosphorylation. In an embodiment, the SAR comprises a signaling adaptor that induces protein dephosphorylation. In an embodiment, the SAR comprises a signaling adaptor that interacts with Zap70. In an embodiment, the SAR comprises a signaling adaptor that does not interact with Zap70. In an embodiment, the two chains of a double chain SAR comprise identical signaling adaptors (e.g., CD3ƒ and CD3ƒ). In an embodiment, the two chains of a double chain SAR comprise non-identical signaling adaptors (e.g., CD3ƒ and FcRγ or CD3ƒ and DAP10 or DAP10 and DAP12 etc.).

In some embodiments, according to any of the double chain SARs comprising signaling adaptors (such as isolated SARs) described above, one or both chains of a double chain SAR comprise signaling adaptors that contain costimulatory domains (e.g., co-stimulatory domains derived from 4-1BB, CD28, 2B4, OX40 etc.). In an embodiment, one or both chains of a double chain SAR comprise signaling adaptor that contain costimulatory domains (e.g., co-stimulatory domains derived from 4-1BB, CD28, 2B4, OX40 etc.) that are operationally linked to activation domains (e.g., CD3ƒ activation domains). Exemplary such CD3 signaling adaptors that are linked to the costimulatory domain of CD28 and 4-1BB are presented in SEQ ID NO:3493 and 3494, respectively. Exemplary SARs comprising the costimulatory domain of OX40 fused to activation domain of CD3 are represented by SEQ ID NO: 4460 and 4479. In an embodiment, one or both chains of a double chain SAR comprise a signaling adaptor containing fusion of the cytosolic domains of two different signaling adaptors. An exemplary such a SAR comprising a chain with containing fusion of cytosolic domains of DAP10 and CD3 is represented by SEQ ID NO: 4460. In an embodiment, the SAR comprises signaling adaptors comprising mutants of CD3z (or CD3ƒ), FcRγ, DAP10 and DAP12 that carry mutations which abolish the interchain disulfide bonds. Exemplary such signaling adaptors are represented by SEQ ID NO:3747, 3753, 3760, 3817 and 3820. In an embodiment, the signaling chain comprise mutants of CD3z, FcRγ, DAP10 and DAP12 that carry one or more mutations in their ITAM motifs (e.g., IXX mutant of CD3z). Exemplary such a signaling adaptor is represented by SEQ ID NO: 9824.

The exemplary antigen binding domains that can be used in the construction of a double chain SAR of the disclosure comprising include variable domains of an antibody (e.g., vL, vH), variable domains of TCR (e.g., Va, Vb, Vg or Vd chains etc.), an antibody, antibody fragment (e.g., Fab, Fab2), autonomous antigen binding domain (e.g., fully human vH domain, vHH, single chain TCR, svd-TCR etc.), scFv, non-immunoglobulin antigen binding domain (e.g., Centyrin, affibody, ZIP domain, an adaptor etc.), ligand, and extracellular domain of a receptor, an auto-antigen, TCR, HLA-independent TCR, variable domains of TCR (e.g., Va, Vb, Vg, Vd etc.) or a fragment thereof etc. In further embodiment, an autonomous antigen binding domain (e.g., fully human vH domain, vHH, single chain TCR etc.), non-immunoglobulin antigen binding domain (e.g., Centyrin, affibody etc.), ligand (e.g., APRIL, TPO, NKG2D-YA-G4Sx3-NKG2D-YA etc.), and extracellular domain of a receptor (e.g., NKp30, NKp44, NKp46, NKG2D, CD16A etc.), an adaptor binding domain (e.g., EZip, RZip, E4, R4 etc.) can be operationally linked the amino-terminus or near the amino-terminus of the vL, vH, Vα, Vβ, Vγ or Vδ chains of the SAR to confer additional antigen binding capabilities on the SAR.

In an embodiment, the two signaling adaptors of a double chain SAR are of the same type (e.g., both chains are derived from CD3ƒ). An exemplary such SAR is represented by SEQ ID NO: 4702. In an embodiment, the two signaling adaptors comprising a double chain SAR are of the different type (e.g., one signaling adaptor is derived from CD3 and the second adaptor is derived from FcRγ etc.). An exemplary such SAR is represented by SEQ ID NO: 6733.

In an embodiment, a double chain SAR may comprise one chain that is derived from a non-TCR receptor signaling chain (e.g., CD16A) and another chain that is derived from a signaling adaptor (e.g., CD3ƒ or FcRy). An exemplary such SAR is represented by SEQ ID NO: 4670.

In an embodiment, a double chain SAR may comprise one chain that comprises a signaling adaptor (e.g., CD3ƒ) and another chain that comprises a TCR constant chain (e.g., TCRa-T48C).

In an embodiment, the optional linker is a long linker. In an embodiment, the optional linker between the vL/vH, Vα/Vβ, Vγ/Vδ chains of the heterologous antigen binding domain and the non-TCR signaling chains is an Ig like linker (SEQ ID NO (DNA): 1142-1175 and SEQ ID NO (PRT): 3536-3569) represented in Table 13.

In some embodiments, the disclosure provides a cell that is not a T cell with target recognition properties and function of a T cell. In an embodiment, the disclosure provides a cell that is not a T cell (i.e., non-T cell) which expresses a receptor that confers on the cell a T cell receptor like target recognition and/or signal transduction. In an embodiment, the disclosure provides a cell that is not a T cell (i.e., non-T cell) which expresses a double chain or a multi-chain receptor that confers on the cell a T cell receptor like target recognition and/or signal transduction. In an embodiment, a double chain or a multichain receptor comprises at least two membrane associated domains (e.g., transmembrane domain or membrane anchoring domain). In an embodiment, a double chain or a multichain receptor comprises at least two transmembrane domains. In an embodiment, T cell receptor like recognition comprises specific binding to a peptide target presented by an MHC molecule. In an embodiment, the cell that is not a T cell (i.e., non-T cell) lacks the expression of T cell chains and/or lacks the expression of functional TCR chains. In an embodiment, the cell that is not a T cell (i.e., non-T cell) lacks the expression of a functional TCR/CD3 complex. In an embodiment, the cell that is not a T cell (i.e., non-T cell) lacks the expression of one or more of CD3ε, CD3γ and CD3δ or variants or fragments thereof. In an embodiment, the cell that is not a T cell (i.e., non-T cell) is not engineered to exogenously express one or more of CD3ε, CD3γ and CD3δ chains or variants or fragments thereof. In an embodiment, the cell that is not a T cell (i.e., non-T cell) is not engineered to exogenously express one or more of TCR chains or variants or fragments thereof. In an embodiment, the cell that is not a T cell (i.e., non-T cell) is not activated by a CD3 agonist antibody. In an embodiment, the cell that is not a T cell (i.e., non-T cell) is not activated by OKT3 antibody.

In an embodiment, the disclosure provides a non-T cell with T cell receptor like target recognition that is generated from an NK cell, g-NK cell, memory like NK cells, cytokine induced killer cell (CIK), iPSC, a modified HLA deficient iPSC, iPSC-derived NK cell, iPSC-derived T cell, B cell, a macrophage/monocyte, granulocyte, a dendritic cell, an immortalized cell line, an immortalized NK cell line, NK92 cell line, NK92MI cell line or derivative thereof. In an embodiment, the disclosure provides a non-T cell with T cell receptor like target recognition that is generated following the introduction of a single receptor into a cell that is not a functional T cell. In an embodiment, the disclosure provides a non-T cell with T cell receptor like target recognition that is generated without genetic modifications involving ectopic expression of the four CD3 chains, i.e., CD3ε, CD3γ, CD3δ and CD3ƒ into a cell that is not a functional T cell.

In an embodiment, the non-T cell expressing the double chain receptor (e.g., a SAR, e.g., uTCR-SAR) upon specific binding the target antigen results in the recruitment of at least one signaling adaptor. In an embodiment, the non-T cell expressing the double chain receptor upon specific binding the target antigen results in activation of at least one signaling pathway. In an exemplary embodiment, the signaling pathway is selected from the group of but not limited to NFAT, NF-κB, PI3K or ERK pathway. In an embodiment, the non-T cell expressing the double chain receptor upon binding the target antigen results in activation of at least one biological activity. In an embodiment, the biological activity is chosen from the group of but not limited to cellular activation, proliferation, differential, cytokine secretion, phagocytosis, migration or cytotoxicity.

In another aspect, the disclosure provides a modified cell, such as, but not limited to, a Natural Killer (NK) cell, having Major Histocompatibility Complex (MHC)-restricted antigen-specific cytotoxicity. The MHC can be any of MHC-class I, MHC-class II, and MHC-like molecules. A non-limiting example of an MHC-like molecule is HLA-E.

In a further aspect, the disclosure provides a method for producing a modified cell, such as, but limited to, a Natural Killer (NK) cell or macrophage, expressing a double chain receptor with two transmembrane/membrane associated domains and TCR like antigen recognition. The method includes providing a cell (e.g., Natural Killer (NK) cell or macrophage) and modifying the cell to express the antigen-specific receptor with TCR like binding properties. The modified cell can be any cell. Commonly used, non-limiting examples, are an NK-92 cell, a YTS cell, and a primary human NK cell.

In another embodiment, the disclosure provides a class of SARs with TCR like binding properties that can be expressed in any cell type. Such a SAR with TCR like binding properties and universal expression is designated Universal TCR-SAR or uTCR-SAR or uTCR. Provided herein are single chain and multichain (e.g., double chain) uTCR-SARs comprising the variable antigen binding domains of a TCR (e.g., Va/Vα, Vb/Vβ, Vg/Vγ, Vd/Vδ etc.) that can be expressed in not only T cells but also in any other cells including, but not limited to, NK cells, monocytes, macrophages, dendritic cells, granulocytes, endothelial cells and epithelial cells etc. In an embodiment, the cells expressing the uTCR respond to target cells expressing their antigen by increased cell proliferation, activation, cytokine secretion and cytotoxicity. In an embodiment, the target antigen is a peptide that is presented as part of an MHC complex. In an embodiment the target antigen is a lipid. The uTCR may also respond to a non-MHC restricted antigen if their TCR binding domain is derived from an HLA-independent TCR. In an embodiment, the disclosure provides a cell that is not a T cells which functionally expresses a double chain receptor with TCR like binding properties, including the property to bind to an intracellular peptide when presented by MHC complex.

The disclosure provides single chain uTCR-SAR comprising one or more heterologous antigen binding domains comprising scTCR, svd-TCR or a TCR mimic scFv or a fragment thereof operationally linked via optional linkers to the entire or partial extracellular domain of a non-TCR signaling receptor. In some embodiments, the disclosure provides that single chain SAR comprising scTCR, svd-TCR or a TCR mimic scFv acquires TCR-like binding capabilities (e.g., ability to bind to a peptide/MHC complex) and can be expressed in any cell, including a cell that is not a T cells or expresses TCR chains.

In some embodiments, there is provided a uTCR-SAR (such as an isolated uTCR-SAR) that specifically binds to a target antigen, wherein the uTCR-SAR comprises: a) a first polypeptide chain comprising a first antigen-binding domain and a first Membrane associated module (MAM); and b) a second polypeptide chain comprising a second antigen-binding domain and a second Membrane associated module (MAM), wherein the first antigen-binding domain and the second antigen-binding domain form a TCR-like (e.g., TCR-Fv) antigen-binding module that specifically binds to the target antigen, and wherein the first MAM and the second MAM form a non-T cell receptor module (NTCRM). In an embodiment, the NTCRM is capable of activating at least one signaling pathway and/or recruiting at least one signaling adaptor.

In an embodiment, the first and second antigen binding domains of the uTCR-SAR comprise of antigen binding domains of a TCR. In an embodiment, the first and second antigen binding domains of the uTCR-SAR comprise of antigen binding domains of a TCR that specifically bind to a peptide presented by an MHC molecule. In an embodiment, the first and second antigen binding domains comprise of variable domains of a TCR. In an embodiment, the first and second antigen binding domains comprise of Vα, Vβ, Vγ and V6 domains of a TCR. In an embodiment, the first antigen binding domain comprises of a Vu domain or a variant or a fragment thereof and the second antigen binding domain comprises of a Vβ domain or a variant or a fragment thereof. In an embodiment, the first antigen binding domain comprises of a Vγ domain or a variant or a fragment thereof and the second antigen binding domain comprises of a Vδ domain or a variant or a fragment thereof. In an embodiment, the first antigen binding domain comprises of antigen binding domain of preTCRα or a variant or a fragment thereof and the second antigen binding domain comprises of a Vβ domain or a variant or a fragment thereof. In an embodiment, the target antigen is a peptide/MHC complex. In an embodiment, the peptide recognized by the uTCR-SAR is an intracellular peptide. In an embodiment, the target antigen of a uTCR-SAR is an MHC-independent antigen. In an embodiment, the target antigen is a lipid. In an embodiment, the uTCR-SAR comprises of the variable domain of an HLA-independent TCR and its target antigen is a cell surface protein. In some embodiments, the uTCR-SAR binds to the target antigen with an equilibrium dissociation constant (Kd) from about 0.1 pM to about 500 nM.

In an embodiment, the first antigen binding domain and the second antigen binding domain are not derived from an antibody or an antibody fragment. In an embodiment, the first antigen binding domain and the second antigen binding domain are not variable domains of an antibody or variants or fragments thereof. In an embodiment, the first antigen binding domain and the second antigen binding domain are not vL and vH domains of an antibody. In an embodiment, the first antigen binding domain and the second antigen binding domain are not vL and vH domains of a TCR mimic antibody.

In an embodiment, the TCR-like (e.g., TCR-Fv) antigen-binding module specifically binds to a peptide presented by an MHC molecule. In an embodiment, the TCR-like (e.g., TCR-Fv) antigen-binding module specifically binds to an antigen (e.g., HLA-independent antigen) that is not presented by an MHC molecule. In an embodiment, the TCR-like (e.g., TCR-Fv) antigen-binding module specifically binds to lipid antigen.

In an embodiment, at least one of the MAM of a uTCR-SAR comprise of the transmembrane domain of a signaling adaptor. In an embodiment, both of the MAM of a uTCR-SAR comprise of the transmembrane domain of a signaling adaptor. In an embodiment, at least one of the MAM of a uTCR-SAR comprise of the transmembrane domain/membrane associated domain of a signaling receptor that is capable of recruiting a signaling adaptor. In an embodiment, both of the MAM of a uTCR-SAR comprise of the transmembrane domain/membrane associated domain of a signaling receptor that is capable of recruiting a signaling adaptor.

In some embodiments, there is provided a uTCR-SAR (such as an isolated uTCR-SAR) that specifically binds to a target antigen, wherein the uTCR-SAR comprises: a) a first polypeptide chain comprising a first antigen-binding domain comprising a Vα or a Vy domain and a first Membrane associated module (MAM); and b) a second polypeptide chain comprising a second antigen-binding domain comprising a Vβ or a Vδ domains and a second Membrane associated module (MAM), wherein the Vα or Vγ domain of the first antigen-binding domain and the complementary Vβ or Vδ domain of the second antigen-binding domain form TCR-like (e.g., TCR-Fv) antigen-binding module that specifically binds to the target antigen, and wherein the first MAM and the second MAM form a non-T cell receptor module (NTCRM). In an embodiment, the NTCRM is capable of activating at least one signaling pathway and/or recruiting at least one signaling adaptor. In an embodiment, the target antigen is a peptide/MHC complex.

In some embodiments, there is provided a uTCR-SAR (such as an isolated uTCR-SAR) that specifically binds to a target antigen, wherein the uTCR-SAR comprises: a) a first polypeptide chain comprising a first antigen-binding domain comprising a Vα domain (variable domain derived from a TCRα chain) and a first Membrane associated module (MAM); and b) a second polypeptide chain comprising a second antigen-binding domain comprising a Vβ domain (variable domain derived from TCRβ chain) and a second Membrane associated module (MAM), wherein the Vα domain of the first antigen-binding domain and the Vβ of the second antigen-binding domain form TCR-like (e.g., TCR-Fv) antigen-binding module that specifically binds to the target antigen, and wherein the first MAM and the second MAM form a non-T cell receptor module (NTCRM). In an embodiment, the NTCRM is capable of activating at least one signaling pathway and/or recruiting at least one signaling adaptor. In an embodiment, the target antigen is a peptide/MHC complex. In an embodiment, the target antigen is an MHC (HLA)-independent antigen.

In some embodiments, there is provided a uTCR-SAR (such as an isolated uTCR-SAR) that specifically binds to a target antigen, wherein the uTCR-SAR comprises: a) a first polypeptide chain comprising a first antigen-binding domain comprising a Vγ domain (variable domain derived from a TCRγ chain) and a first Membrane associated module (MAM); and b) a second polypeptide chain comprising a second antigen-binding domain comprising a Vδ domain (variable domain derived from TCRS chain) and a second Membrane associated module (MAM), wherein the Vγ domain of the first antigen-binding domain and the Vδ of the second antigen-binding domain form TCR-like (e.g., TCR-Fv) antigen-binding module that specifically binds to the target antigen, and wherein the first MAM and the second MAM form a non-T cell receptor module (NTCRM). In an embodiment, the NTCRM is capable of activating at least one signaling pathway and/or recruiting at least one signaling adaptor. In an embodiment, the target antigen is a peptide/MHC complex. In an embodiment, the target antigen is an MHC (HLA)-independent antigen. In an embodiment, the target antigen is a lipid.

In some embodiments, there is provided a uTCR-SAR (such as an isolated uTCR-SAR) that specifically binds to a target antigen, wherein the uTCR-SAR comprises: a) a first polypeptide chain comprising a first antigen-binding domain comprising a V-preTCRα domain (variable domain derived from a preTCRα chain) and a first Membrane associated module (MAM); and b) a second polypeptide chain comprising a second antigen-binding domain comprising a Vβ domain (variable domain derived from TCRβ chain) and a second Membrane associated module (MAM), wherein the Vα domain of the first antigen-binding domain and the Vβ of the second antigen-binding domain form TCR-like (e.g., TCR-Fv) antigen-binding module that specifically binds to the target antigen, and wherein the first MAM and the second MAM form a non-T cell receptor module (NTCRM). In an embodiment, the NTCRM is capable of activating at least one signaling pathway and/or recruiting at least one signaling adaptor. In an embodiment, the target antigen is a peptide/MHC complex.

In an embodiment, the first MAM and the second MAM do not comprise the transmembrane domain of a TCR chain selected from TCRα, TCRβ, TCRγ, TCRS or preTCRα. In an embodiment, the first MAM or the second MAM do not comprise the transmembrane domain of a TCR chain selected from TCRα, TCRβ, TCRγ, TCRS or preTCRα. In an embodiment, the first MAM and the second MAM do not comprise the transmembrane domain of a CD3 chain selected from CD3ε, CD3γ, CD3δ or CD3ƒ. In an embodiment, the first MAM and the second MAM do not comprise the transmembrane domain of a TCR chain and a CD3 chain. In an embodiment, the first MAM and the second MAM do not comprise the transmembrane domain of CD3ƒ. In some embodiments, the first and the second MAM of a uTCR-SAR comprises of a transmembrane or membrane associated domain of a signaling adaptor. In an embodiment, the signaling adaptor is selected from, but not limited, to one or more of CD3ƒ, FcRγ, DAP10 and/or DAP12 or variants or fragments thereof. In some embodiments, the signaling adaptor is a non-CD3 adaptor (NCAM). In some embodiments, the signaling adaptor is not CD3ƒ. In an embodiment, the MAM of a uTCR-SAR comprises of a non-TCR receptor. In an embodiment, the non-TCR is selected from, but not limited to, one or more of the following: CD16A, CD16B, CD64, CD32, NKp30, NKp44, NKp46, KIR2DL1, KIR2DL2, KIR2DL3, KIR2DL5A, KIR2DL5B, KIR3DL1, KIR3DL2, KIR3DL4, KIR2DL4, KIR2DS1, KIR2DS2, KIR2DS3, KIR2DS4, KIR2DS5, KIR3DS1, DNAM-1, 2B4, OX40, CD28, 4-1BB, CD27, CD81, CD2, CD5, TNFR-I, TNFR-II, Fas, CD30, CD40, CRTAM, TIGIT, CD96, SLAMF6, SLAMF7, CD100, CD160, and ILT2. Exemplary uTCR-SAR comprising variable domains of a NY-ESO-1 TCR attached to two polypeptides comprising the hinge domains of NKp46 and CD16 and NKp30 are represented by SEQ ID NO:10467 and 10468, respectively. An exemplary uTCR-SAR comprising Va and Vb domains of a NY-ESO-1 TCR attached to two polypeptides comprising the extracellular domain of NKp30 is represented by SEQ ID NO: 10469. In some embodiments, the first polypeptide chain and the second polypeptide chain are linked via one or more disulfide bonds. In some embodiments, the first polypeptide chain further comprises a first peptide linker between the first antigen-binding domain and the first MAM. In some embodiments, the second polypeptide chain further comprises a second peptide linker between the second antigen-binding domain and the second MAM. In some embodiments, the first polypeptide chain and the second polypeptide chain are linked via one or more disulfide bonds. In some embodiments, the first peptide linker and/or the second peptide linker are, individually, from about 5 to about 500 amino acids in length. In some embodiments, the first and/or second peptide linkers comprise, individually, a constant domain or fragment thereof from an immunoglobulin or T cell receptor subunit. In some embodiments, the first and/or second peptide linkers comprise, individually, a CH1, CH2, CH3, CH4 or CL antibody domain, or a fragment thereof. In some embodiments, the first and/or second peptide linkers comprise, individually, a Cα, Cβ, Cγ, or Cδ TCR domain, or a variant or a fragment thereof. In exemplary embodiments, the first and/or second linkers comprise, individually, an Ig like linker (e.g., IgCL, IgCH1 etc.) derived from an immunoglobulin (e.g., SEQ ID NO: 3536-3551) or a TCR-Ig like linker (e.g., TCRb-Ig3, SEQ ID NO: 3560; TCRa-Ig3, SEQ ID NO:3562; TCRg-Ig3, SEQ ID NO: 3566; or TCRd-Ig3, SEQ ID NO: 3568 etc.) or a variant or a fragment thereof. In some embodiments, first and/or second peptide linkers comprise mutations that increase their expression, affinity and chain pairing. In an embodiment, the first and the second antigen binding domains comprise complementary chains (e.g., Vα and Vβ or Vγ and Vδ). In some embodiments, the first and the second polypeptide chains further comprise one or more autonomous antigen binding domains (AABD) that are attached to the N-terminus or near the N-terminus of the first (e.g., vL, Vα or Vγ) and/or the second (e.g., vH, Vβ or Vδ) antigen binding domains. In some embodiments, the SAR binds to the target antigen with an equilibrium dissociation constant (Kd) from about 0.1 pM to about 500 nM. In some embodiments, the target antigen is a complex comprising a peptide and a major histocompatibility complex (MHC) protein. In exemplary embodiments, the peptide/MHC complex comprises a peptide derived from one or more of NY-ESO-1, MAGE-A2, MAGE-A3, MAGE4, WT1, AFP, TERT, MART-1, pp66-CMV, HPV16-E7, PRAME, EBV-LMP2A, HIV-1, PSA or gp100. In some embodiments, the uTCR is an HLA independent TCR that can target cell surface antigens. In an embodiment, the target antigen is a cell surface antigen. In an exemplary embodiment, the target antigen is one or more of the antigens listed in Table B. In some embodiments, the cell surface antigen is selected from the group consisting of protein, carbohydrate, and lipid. In some embodiments, the cell surface antigen is one or more of CD2, CD5, CD19, CD20, CD22, CD33, CD70, CD123, CD138, CD179b, CLL-1, FLT3, Claudin 18.2, BCMA, GCC, MPL, SLAMF7, ROR1, ROR2, GPRC5D, FCRL5, MSLN, EGFR, EGFRviii, PSMA, PSCA, KLK2, IL13Ra2, TROP2, PTK7, DLL3, Mucd, Muc16 or Her2. In an embodiment, a uTCR-SAR is bispecific or multispecific. In an embodiment, the disclosure provides a uTCR that can bind to two or more antigens that are MHC restricted. In an embodiment, a uTCR-SAR can bind to two or more antigens that are MHC restricted and/or MHC-non-restricted. In an embodiment, a uTCR-SAR can bind to a peptide/MHC complex via its TCR-Fv domain and bind to one or more peptide/MHC complexes via one or more svd-TCR that are attached to the N-terminus or near the N-terminus of its Vα and Vβ or Vγ and Vδ domains. In an embodiment, a uTCR-SAR can bind to one or more peptide/MHC complex via its TCR-Fv domain and svd-TCR domain and bind to one or more surface antigens via one or more AABD (e.g., vHH, FHVH, centyrin etc.) that are attached to the N-terminus or near the N-terminus of its Vα and Vβ or Vγ and Vδ domains.

In some embodiments, according to any of the uTCR-SARs (such as isolated uTCR-SARs) described above, the first MAM further comprises a first hinge domain or fragment thereof N-terminal to the first transmembrane domain, and/or the second MAM further comprises a second hinge domain or fragment thereof N-terminal to the second transmembrane domain. In some embodiments, the NTCRM comprises a disulfide bond between a residue in the first hinge domain and a residue in the second hinge domain. In some embodiments, according to any of the uTCR-SARs (such as isolated uTCR-SARs) described above, the first MAM further comprises a first antigen binding domain or fragment thereof N-terminal to the first hinge domain and/or the second MAM further comprises a second antigen binding domain or fragment thereof N-terminal to the second hinge domain. In some embodiments, the first MAM further comprises a first cytosolic domain C-terminal to the first transmembrane domain. In some embodiments, the second MAM further comprises a second cytosolic domain C-terminal to the second transmembrane domain. In an embodiment, the first and/or second cytosolic domains are activation domains comprising one or more ITAMs. In some embodiments, the uTCR-SAR binds to the target antigen with an equilibrium dissociation constant (Kd) from about 0.1 pM to about 500 nM.

In some embodiments, according to any of the uTCR-SARs (such as isolated uTCR-SARs) described above, the first polypeptide chain further comprises a first co-stimulatory domain C-terminal to the first transmembrane domain. In some embodiments, the second polypeptide chain further comprises a second co-stimulatory domain C-terminal to the second transmembrane domain. In some embodiment, according to any of the uTCR-SARs (such as isolated uTCR-SARs) described above, the first polypeptide chain comprises more than one co-stimulatory domains C-terminal to the first transmembrane domain and/or the second polypeptide chain comprises more than one co-stimulatory domains C-terminal to the second transmembrane domain. In some embodiments, the first polypeptide chain further comprises a first signaling peptide N-terminal to the first antigen-binding domain. In some embodiments, the second polypeptide chain further comprises a second signaling peptide N-terminal to the second antigen-binding domain.

In an embodiment, the disclosure provides a double chain uTCR-SAR construct (such as an isolated construct) that specifically targets an antigen (e.g., a peptide/MHC complex) comprising TCR variable domains (e.g., Va/Vα, Vb/Vβ, Vg/Vγ, Vd/Vδ etc.) fused to at least one polypeptide comprising a non-T cell receptor module (NTCRM). In some embodiments, the SAR comprises one or more TCR variable domains that specifically bind to a target antigen (e.g., a peptide/MHC complex or a lipid antigen) and a non-T cell receptor module (NTCRM) capable of recruiting at least one signaling adaptor. In an exemplary embodiment, the signaling adaptor is one or more of but not limited to CD3ƒ, FcRγ, DAP10 or DAP12. In some embodiments, the target antigen is a complex comprising a peptide and an MHC protein (such as an MHC class I protein or an MHC class II protein).

In an embodiment, the uTCR-SAR is expressed on the surface of a cell. In an embodiment, the uTCR-SAR is expressed on the surface of a cell that is not a T cell. In an embodiment, the uTCR-SAR is expressed on the surface of a cell that lacks the expression of TCRα, TCRβ, TCRγ, TCRS, preTCRα chains or variants or fragments thereof. In an embodiment, the uTCR-SAR is expressed on the surface of a cell that lacks the expression of CD3ε, Cd3γ and CD3δ chains or variants or fragments thereof. In an embodiment, the uTCR-SAR with TCR-like properties is functionally active (i.e., capable of inducing cell proliferation, cytokine secretion or cytotoxicity) when expressed in a T cell. In an embodiment, the uTCR-SAR is functionally active (i.e., capable of inducing cell proliferation, cytokine secretion or cytotoxicity) when expressed in a cell that is not a T cell (i.e., when expressed in a NK cell, macrophage, granulocyte dendritic cell etc.). In an embodiment, the SAR is expressed and functionally active in a cell that lacks the expression of one or more of TCRα, TCRβ, TCRγ, TCRS and preTCRα chains or variants or fragments thereof. In an embodiment, the uTCR-SAR is expressed and functionally active in a cell that lacks the expression and/or function of TCRα, TCRβ, TCRγ, TCRS and preTCRα chains or variants thereof. In an embodiment, the uTCR-SAR is expressed and functionally active in a cell that lacks the expression and/or function of CD3ε, Cd3γ and CD3δ chains or variants thereof.

In an embodiment, a uTCR-SAR confers TCR-like antigen recognition to a T cell. In an embodiment, a uTCR-SAR confers TCR-like antigen recognition to a cell other than a T cell (e.g., NK cell, g-NK cell, memory like NK cell, CIK, monocytes, macrophages, dendritic cells, epithelial cells, iPSC derived NK cells etc.). In an embodiment, a uTCR-SAR is capable of binding peptide antigens in an MHC (HLA) dependent manner. In an exemplary embodiment, a cell (e.g., NK cell or macrophage) expressing a uTCR-SAR can recognize intracellular peptide antigens in an MHC (HLA)-dependent manner. In an exemplary embodiment, an immune cell (e.g., NK cell or macrophage) expressing a uTCR-SAR can recognize intracellular peptide antigens in an MHC (HLA)-dependent manner and activate one or more cellular signaling pathways (e.g., NFAT, PI3K, NF-κB pathway etc.). In an exemplary embodiment, an immune cell (e.g., NK cell or macrophage) expressing a uTCR-SAR can recognize intracellular peptide antigens in an MHC (HLA)-dependent manner and block one or more cellular signaling pathways (e.g., NFAT, PI3K, NF-κB pathway etc.). In an embodiment, an immune cell (e.g., NK cell, T cell or macrophage) expressing a uTCR/SAR possess the ability to induce cell activation, proliferation, cytokine secretion (e.g., secretion of IFNγ, TNFα and IL2) and/or cytotoxicity upon binding their target peptide antigen. In an embodiment, an immune cell (e.g., NK cell, T cell or macrophage) expressing a uTCR-SAR possess the ability to block cell activation, proliferation, cytokine secretion (e.g., secretion of IFNγ, TNFα and IL2) and/or cytotoxicity upon binding their target peptide antigen. In an embodiment, a uTCR-SAR is an activating receptor. In an embodiment, uTCR-SAR is an inhibitory receptor.

In an embodiment, a uTCR-SAR comprises one or more antigen binding domains derived from variable domains of a TCR (e.g., Va/Vα, Vb/Vβ, Vg/Vγ, Vd/Vδ and preTCRα) and has two chains. In an embodiment, a uTCR-SAR comprises a Va/Vα and a Vb/Vβ domain. In an embodiment, a uTCR-SAR comprises a Vg/Vγ and a Vd/Vδ domain. In an embodiment, the uTCR-SAR comprises a preTCRα and a Vb/Vβ domain. In an embodiment, the two variable domains of a uTCR SAR are present on two separate polypeptide chains. In an embodiment, the two variable domains comprising the antigen binding domain (e.g., peptide/MHC complex binding domain) of a uTCR SAR are not part of a single polypeptide chain. In an embodiment, the two variable domains of a uTCR-SAR are not operationally linked via a linker, i.e., a uTCR is not a single chain TCR (scTCR).

In an embodiment, one or both chains of a uTCR comprise a transmembrane domain or a membrane anchoring domain. In an embodiment, one or both chains of a uTCR SAR comprise a cytosolic domain. In an embodiment, one or both chains of a uTCR SAR comprise transmembrane and/or cytosolic domains that are capable of recruiting signaling proteins or signaling adaptors. In an embodiment, one or both chains of a double chain uTCR SAR comprise one or more cytosolic activation domains. In an embodiment, one or both chains of a double chain uTCR SAR comprise one or more ITAMs in their cytosolic domain. In an exemplary embodiment, one or both chains of a double chain uTCR SAR comprise cytosolic domains comprising 1, 2 or 3 ITAMs. In an exemplary embodiment, one chain of a uTCR SAR comprises a cytosolic domain with a single ITAM while the second chain has a cytosolic domain with 3 ITAMs. In an exemplary embodiment, one chain comprises a cytosolic domain with a single 1 ITAM while the second chain comprises a cytosolic domain with 2 or 3 ITAMs. In an embodiment, one or both chains of a double uTCR SAR comprise one or more inhibitory motifs, e.g., ITIMs. In an embodiment, the uTCR-SAR comprises a cytosolic activation domain derived from a CD3 chain in which 1 or more ITAMs are mutated. In exemplary embodiment, the uTCR-SAR comprises a cytosolic activation domain derived from a CD3 chain in which the tyrosine residues of 1 or more ITAMs are mutated to phenylalanine.

In an embodiment, the disclosure also provides uTCR-SAR comprising variable domains (e.g., Va, VD, Vγ and VS etc.) of TCRs as their antigen binding domains operationally linked to the extracellular domains of signaling chains (adaptors) and/or non-TCR receptors and comprising co-stimulatory domains.

In an embodiment, one or both chains of a double chain uTCR SAR with TCR-like binding properties optionally comprise one or more co-stimulatory domains. In an embodiment, the one or more co-stimulatory domains are located in the juxtamembrane regions of one or both chains. In an exemplary embodiment, the co-stimulatory domains are derived from the cytosolic domain of 4-1BB, CD28, CD27, CD81, OX40, 2B4 or CD2 etc. Exemplary such CD3 signaling chains comprising the costimulatory domain of CD28 and 4-1BB are presented in SEQ ID NO:3493 and 3494, respectively. As SARs are modular in format, the costimulatory domains of CD28 and 4-1BB can be replaced by co-stimulatory domains derived from other co-stimulatory receptors (e.g., OX40, 2B4, CD2, CD81 etc.) and variants thereof to generate novel uTCR-SARs. Similarly, one or both CD3 signaling chains can be substituted for other signaling chains to generate novel uTCR-SARs based on the signaling chains of FcRγ, DAP10 and DAP10 and variants thereof and comprising the different costimulatory domains. Exemplary such uTCR-SAR targeting NY-ESO1 peptide/MHC complex are represented by SEQ ID NO: 10481-10530.

In an embodiment, the uTCR SAR with TCR-like binding properties is unispecific. In an embodiment, the uTCR SAR with TCR-like binding properties is bispecific. In an embodiment, the uTCR SAR with TCR-like binding properties is biparatopic. In an embodiment, the uTCR SAR with TCR-like binding properties is multispecific. In an embodiment, the disclosure provides uTCR SAR with TCR like binding properties that are capable of binding to two or more distinct intracellular peptides when presented by the MHC complex. In an embodiment, the disclosure provides uTCR SAR with TCR like binding properties that are capable of binding to intracellular peptides and cell surface expressed (or extracellular) proteins (e.g., CD19, CD20 etc.). An exemplary such uTCR-SAR that targets both NY-ESO-1 peptide/MHC complex and CD20 is represented by SEQ ID NO:10478. In this construct a vHH domain targeting CD20 is attached to the N-terminus of Vb domain targeting NY-ESO-1 peptide via a small linker. The CD20 vHH domain can be replaced by other AABD targeting other surface antigens or peptide/MHC complexes. In an exemplary embodiment, the CD20 vHH domain is replaced by a single variable domain TCR targeting a MAGE-A3 peptide/HLA-A2 complex to generate a bispecific uTCR-SAR that can target both NY-ESO-1 and MAGE-A3 peptides. An AABD can be also attached to the Va domain of a uTCR to generate bispecific SAR or to both Vb and Va domains to generate a multispecific uTCR-SAR. Further, more than one AABD (e.g., vHH, FHVH, centyrins, svd-TCR) can be attached to the N-terminus of each of the variable domains of a uTCR-SAR. In an embodiment, the disclosure provides uTCR SAR with TCR like binding properties that are capable of binding to their target antigen(s) in an MHC (or HLA)-dependent and an MHC (HLA)-independent manner.

In an embodiment, the uTCR SAR with TCR binding properties comprise two variable domains (e.g., Vα and Vβ or Vγ and Vδ etc.) that associate with each other to form a fragment variable TCR (TCR-Fv) that binds to the peptide/MHC complex. In an embodiment, the SAR with TCR binding properties further comprise one or more autonomous antigen binding domains (e.g., vHH, FHVH, svd-TCR etc.). In an embodiment, the one or more autonomous antigen binding domains (e.g., vHH, FHVH, svd-TCR etc.) are operationally linked to the N-terminus or near N-terminus of one or both of the TCR variable domains (e.g., Vα and Vβ or Vγ and Vδ etc.) via optional linkers. In exemplary embodiments, the disclosure provides double chain SARs that recognize NY-ESO-1 peptide in complex with MHC through their Va/V domains and co-expresses svd-TCR targeting NY-ESO-1 or MAGE-A3 or a vHH or FHVH domain targeting CD20 or BCMA etc. An exemplary such uTCR-SAR that targets NY-ESO-1 peptide/MHC complex, CD20 and BCMA is represented by SEQ ID NO:10479.

In an embodiment, the disclosure provides a double chain uTCR SAR in which the Va (Vα) domain of a TCR is operationally linked to the extracellular domain of one membrane anchored polypeptide chain via an optional linker (e.g., TCRa-Ig3, SEQ ID NO: 3562) and a Vb (Vβ) domain is operationally linked to the extracellular domain of a second membrane anchored polypeptide chain via an optional linker (e.g., TCR-like linker, e.g., TCRb-Ig3; e.g., SEQ ID NO: 3560). In an embodiment, one or both membrane anchored polypeptide chains comprising the double chain SAR are transmembrane proteins.

In an exemplary embodiment, the disclosure provides a uTCR-SAR in which the Va (Vα) domain derived from a TCR is operationally linked to the extracellular hinge domain of one chain of a signaling adaptor (e.g., CD3ƒ, FcRγ, DAP10 or DAP12 etc.) or a variant thereof via an optional linker (e.g., TCRa-Ig3, SEQ ID NO: 3562) and a Vb (Vβ) domain is operationally linked to the extracellular hinge domain of a second chain of a signaling adaptor or a variant thereof via an optional linker (e.g., TCR-like linker, e.g., TCRb-Ig3; e.g., SEQ ID NO: 3560). An exemplary such uTCR-SAR that recognizes an NY-ESO-1 peptide/HLA-A*02:01 complex when expressed in a cell (e.g., T cell, NK cell, macrophage etc.) is presented in SEQ ID NO (DNA): 9355 and SEQ ID NO: (PRT): 10447. As SAR are modular in format, the one or both CD3 signaling chains of this SAR can be replaced by other signaling chains/adaptors, including signaling chains of FcRγ, DAP10 and DAP10 or variants thereof. Furthermore, the linker domains can be replaced by other linker domains. In an exemplary embodiment, the Ig like linker TCRa-Ig3 (SEQ ID NO: 3562) is replaced by IgCL linker (SEQ ID NO: 3536) and the linker TCRb-Ig3 (SEQ ID NO: 3560) is replaced by IgG-CH1 (SEQ ID NO: 3537), IgG2-0C-CH1(SEQ ID NO: 3543), IgG2-IC-CHI1 (SEQ ID NO: 3544), IgG3-CHI1 (SEQ ID NO: 3545), IgG4-CHI1(SEQ ID NO:3546), IgAI-CHI1 (SEQ ID NO: 3547), IgA2-CHI1, IgD-CHI1, IgE-CHI1 or IgM-CHI1 (SEQ ID NO: 3551). Exemplary such uTCR-SAR constructs targeting NY-ESO-1 peptide/MHC complex are represented by SEQ ID NO:9357-9365.

In some embodiments, a Vα domain is attached to an IgCL linker and a VP domain is attached to an IgCH1 linker. In some embodiments, a Vβ domain is attached to an IgCH1 linker and a Vα domain is attached to a IgCL linker. In some embodiments, a Vu domain is attached to a Cu-derived linker (e.g., TCRa-Ig3) and Vβ domain is attached to a CP derived linker (e.g., TCRb-Ig3). In some embodiments, a Vβ domain is attached to a Cu-derived linker (e.g., TCRa-Ig3) and Vα domain is attached to a Cβ derived linker (e.g., TCRb-Ig3). An exemplary such construct is represented by SEQ ID NO: 10448. In some embodiments, a Vγ domain is attached to a Cy-derived linker (e.g., TCRg-Ig3) and Vδ domain is attached to a Cδ derived linker (e.g., TCRd-Ig3). An exemplary such construct is SEQ ID NO:10694. This construct has the Vd2 and Vg9 variable domains. In some embodiments, a Vγ domain is attached to a Cδ-derived linker (e.g., TCRd-Ig3) and Vδ domain is attached to a Cγ derived linker (e.g., TCRg-Ig3). An exemplary such as construct is represented by SEQ ID NO: 10693. In some embodiments, other configurations of variable domains and linkers are envisioned.

In an exemplary embodiment, in the case of a uTCR-SAR comprising the variable domains of TCRγ and TCRδ, the Vγ fragment is attached to one chain of the signaling adaptor (e.g., CD3ƒ, FcRγ, DAP10 or DAP10 etc.) via an Ig-like linker derived from TCRγ (e.g., TCRg-Ig3, SEQ ID NO: 3566) and the Vδ fragment is attached to the second chain of the signaling adaptor via an Ig like linker derived from TCRδ chain (e.g., TCRd-Ig3, SEQ ID NO: 3568). The TCRg-Ig3 and TCRg-Ig3 linker domains can be replaced by other linker domains. The disclosure provides SAR comprising the variable domains of TCRγ and TCRδ in which linkers derived from Ig (e.g., IgCL and IgG-CH1) or TCRα/β (e.g., TCRa-Ig3 and TCRb-Ig3) can be substituted for one or both linkers derived from TCRγ (e.g., TCRg-Ig3, SEQ ID NO: 3566) and TCRδ chain (e.g., TCRd-Ig3, SEQ ID NO: 3568).

The disclosure provides heterodimeric double chain uTCR-SAR comprising variable domains of TCR as their antigen binding domains in which the two signaling chains are of different types (e.g., CD3ƒ and FcγR, CD3ƒ and DAP10, CD3ƒ and DAP12, FcRγ and DAP10 etc.). The disclosure provides heterodimeric double chain uTCR-SAR in which one or both signaling chains comprise the transmembrane and optionally the cytosolic domains of a naturally occurring signaling receptor, e.g., CD16A, NKp30, NKp44, NKp44 etc. The one or both chains of such uTCR-SAR may further comprise one or more co-stimulatory domains. Exemplary uTCR-SAR constructs targeting NY-ESO-1 peptide/HLA-A*02:01 and MAGE-A3 peptide/HLA-A*02:01 complexes and comprising different binding domains, linkers, activation domains and costimulatory domains are represented by SEQ ID NO (PRT): 10447-10530 and 10531-10610, respectively. Exemplary uTCR-SAR constructs comprising the variable domain of MC.7.G5, an HLA-independent TCR, that recognizes multiple cancer types are represented by SEQ ID NO (PRT): 10620-10692 and SEQ ID NO (DNA): 9528-9600.

The disclosure provides, single chain, double chain and double chain hetero-dimeric SARs comprising the partial or entire region of CD16 (FcγRIII). The disclosure provides SARs comprising CD16 or fragments thereof that have 70%, 75%, 80%, 85%, 90%, 95%, 97%, 98%, 98.5%, 99% or 99.9% identity to any of the CD16 sequences described herein while retaining the biological activity. Exemplary full-length CD16 nucleic acid and amino acid sequences that can be used in the construction of CD16-SARs of the disclosure are provided in SEQ ID NO (DNA): 1415-1417 and SEQ ID NO (PRT): 3809-3811 or equivalent residues (i.e., a homolog) from a non-human species, e.g., mouse, rodent, monkey, ape and the like. The CD16 fragments that can be used in the construction of CD16 SARs of the disclosure are provided in Tables 25-30 of the provisional application. The CD16 SARs can be also constructed using variants of the CD16 fragments whose sequences are provided in Tables 25-30 or equivalent residues from non-human species. Exemplary single chain, double chain and double chain hetero-dimeric SARs of the disclosure are provided in Tables 32, 34, and 36-39 of the provisional application.

CD16 has two isoforms, CD16a and CD16b which bears sequence homology in the extracellular and transmembrane domains. Unless specified otherwise, CD16 refers to both CD16a (FcγRIIIa) and CD16b (FcγRIIIb) isoforms and any other alternatively spliced variant from human or non-human species. However, as the CD16b isoform lacks a cytosolic domain, any description regarding the CD16 cytosolic domain pertains only to the CD16a isoform and the equivalent residues from a non-human species. In some embodiments, the CD16 sequences that can be used in the construction of the CD16 SARs of the disclosure may include mutants and variants that increase the affinity of CD16 for immunoglobulin Fc region (e.g., CD16A-F158V; SEQ ID NO: 1415) and, in addition, prevent its cleavage from cell surface (e.g., CD16A-F158V-S197P; SEQ ID NO: 1453).

In certain embodiments, the nucleic acid sequence of the SAR molecule comprises the nucleic acid sequence of human CD16 as shown in SEQ ID NO: 1415-1417. In certain embodiments, the nucleotide sequence of the SAR comprises sequence that encodes for amino acid sequence of CD16 having at least one, five or ten modifications but not more than 20 modifications of an amino acid sequence of SEQ ID NO: 3809-3811, or a sequence with 70-99% identity to an amino acid sequence of SEQ ID NO: 3809-3811. In certain embodiments, SAR molecule comprises the amino acid sequence of SEQ ID NO: 3809-3811 or equivalent residues from a non-human species.

In an embodiment, the disclosure provides a single chain CD16 SAR comprising the partial or entire region of CD16 or a variant thereof. In an embodiment, the disclosure provides a single chain CD16 SAR comprising a partial or entire region of CD16 extracellular domain. Exemplary CD16 extracellular domain sequences that can be used in the construction of a CD16-SAR of the disclosure are provided in SEQ ID NO (DNA): 1496-1509 and SEQ ID NO (PRT): 3890-3903 or the equivalent residues (i.e., a homolog) from a non-human species. In an embodiment, the disclosure provides a CD16 SAR comprising the partial or entire region of CD16 hinge domain. Exemplary CD16 hinge domain sequences that can be used in the construction of a CD16-SAR of the disclosure are provided in SEQ ID NO (DNA): 1545-1547 and SEQ ID NO (PRT): 3939-3941 or the equivalent residues (i.e., a homolog) from a non-human species. In an embodiment, the disclosure provides a CD16 SAR comprising the partial or entire region of CD16 transmembrane domain. Exemplary CD16 transmembrane sequences that can be used in the construction of CD16-SARs of the disclosure are provided in SEQ ID NO (DNA): 1528-1530 and SEQ ID NO (PRT): 3922-3924 or the equivalent residues (i.e., a homolog) from a non-human species. In an embodiment, the disclosure provides a CD16 SAR comprising a partial or entire region of CD16 cytosolic domain. Exemplary CD16 transmembrane sequences that can be used in the construction of CD16-SARs of the disclosure are provided in SEQ ID NO (DNA): 1556-1558 and SEQ ID NO (PRT): 3950-3952 or the equivalent residues (i.e., a homolog) from a non-human species. The disclosure also provides SARs comprising variants of CD16 or fragments thereof that retain at least one biological activity of the wild-type CD16 to which it has identity or homology.

In an embodiment, the CD16 SAR comprises the CD16 extracellular domain comprising both immunoglobulin like domains (i.e., D1 and D2) that is attached via the CD16 hinge domain to CD16 transmembrane domain and to CD16 cytosolic domain. An exemplary such CD16 SAR targeting BCMA is represented by CD8SP-Sph-BCMA-FHVH93-Kpn-G4S-EcoR1-Xho-CD16-F158V-FL-TMCP-v1-F-P2A-SpeXba-PAC (SEQ ID NO(DNA): 1638 and SEQ ID NO (PRT): 4032). Additional exemplary such SARs comprising scFv, FHVH, vHH and non-immunoglobulin antigen binding scaffolds targeting different antigens are provided in SEQ ID NO (DNA): 4851-5121. Such a CD16 SAR also retains the ability to bind to the Fc region of an antibody, an antibody fragment or bispecific/tri-specific engager and mediate antibody dependent cytotoxicity. Thus, immune cells (e.g., T cells, NK cells, monocytes/macrophages, neutrophils etc.) expressing the SAR CD8SP-BCMA-FHVH-33-CD16A-F158V-S197P-FL-v3 (SEQ ID NO: 5062) can target BCMA expressing target cells through BCMA FCVH region. In addition, such immune cells can be redirected to targeted Her2 expressing target cells in the presence of Herceptin. Alternatively, such immune cells (e.g., T or NK cells) can be redirected to targeted CD20 expressing target cells in the presence of Rituximab.

In an embodiment, the CD16 SAR contains the partial CD16 extracellular domain that is missing the first immunoglobulin like domain (i.e., D1) of CD16. Such a CD16 SAR comprises the linker region between D1 and D2 domains and 2nd immunoglobulin like domains (i.e., D2) that is attached via CD16 hinge domain to CD16 transmembrane domain and CD16 cytosolic domain. An exemplary such CD16 SAR targeting BCMA is represented by CD8SP-Sph-BCMA-FHVH93-Kpn-G4S-EcoR1-Xho-CD16-F158V-D2TMCPv1-F-P2A-SpeXba-PAC (SEQ ID NO (DNA): 1664 and SEQ ID NO (PRT): 4058). Such a CD16-SAR lacks the ability to bind to an antibody as it contains only the D2 domain of CD16 and lacks the D1 domain.

In an embodiment, a CD16 SAR comprises the CD16 D2 domain that is attached via CD16 hinge domain to CD16 transmembrane domain and CD16 cytosolic domain. Such a CD16-SAR lacks the ability to bind to an antibody as it contains only the D2 domain of CD16 and lacks the D1 domain.

In an embodiment, the CD16 SAR comprises the partial or entire CD16 hinge domain that is attached to CD16 transmembrane domain and to CD16 cytosolic domain. An exemplary such CD16 SAR targeting BCMA is represented by CD8SP-Sph-BCMA-FHVH93-Kpn-G4S-EcoR1-Xho-CD16-F158V-Hinge-TM-CP-v1-F-P2A-SpeXba-PAC (SEQ ID NO(DNA): 1690 and SEQ ID NO (PRT): 4084). Such a CD16-SAR lacks the ability to bind to an antibody as it lacks both the D1 and D2 domains.

In an embodiment, the CD16 SAR comprises a heterologous hinge (spacer) domain that is present between the antigen binding domain (e.g., scFv, or AABD) and the hinge domain of CD16. An exemplary such CD16 SAR targeting CD19 is represented by CD8SP-CD19-hu-mROO5-1-scFv-CD8-hinge-CD16A-Hinge-TM-CP-V158-F-P2A-PAC (SEQ ID NO (DNA): 7693 and SEQ ID NO (PRT): 8385). This construct comprises a CD19 targeted hu-mROO5-1 scFv operationally linked via CD8-hinge to a fragment encoding CD16A-Hinge, transmembrane and cytosolic domain. In an alternate embodiment, the CD8 hinge region is directly linked to CD16A transmembrane and cytosolic domains. As the SAR are modular in the design, the CD19-hu-mROO5-1-scFv in the above constructs can be replaced by an antigen binding domain (e.g., scFv, AABD etc.) targeting another antigen. Further, the CD8 hinge domain can be replaced by a different hinge domain. An exemplary such construct comprising a CD28 hinge in place of the CD8 hinge is presented by CD8SP-CD19-hu-mROO5-1-scFv-CD28-Ig-113-137-CD16A-v158-Hinge-TM-CP-v2-F-F2A-PAC (SEQ ID NO (DNA): 7683; SEQ ID NO (PRT): 8375) (Table 46).

In an embodiment, the CD16 SAR comprises, an AABD (e.g., a vHH, FHVH, chVH, centyrin, affibody etc.) that is inserted between the D2 domain and the hinge domain of CD16 with optional intervening linkers (e.g., Gly4-Ser linker). In an exemplary embodiment, the different domains of such a CD16 SAR from amino to carboxy-terminal include an N-terminal signal peptide, CD16-D1 domain, CD16-D2 domain, optional linker, AABD (e.g., vHH, FHVH, centyrin, affibody etc.), optional linker, CD16-hinge domain, CD16-transmembrane domain and CD16-cytosolic domain.

It is to be understood that the different CD16 domains (i.e., extracellular, D1, D2, hinge, transmembrane and cytosolic) that may be used in the construction of the SAR may comprise their entire sequence or a deletion mutant or a variant as long as the domain retains at least one of its functional properties. The CD16 domains may comprise their wild-type sequence or one or more of the high affinity (e.g., F158V) or high affinity non-cleavable (e.g., F158V/S197P or F158V/S197R) variants.

In an embodiment, the antigen binding domain of the CD16 SAR comprises a scFv, a vL, vH, Fv, Va, Vb, Vg, Vd, TCR-Fv, vHH, FHVH, a single domain antibody, a single chain TCR (scTCR), a single variable domain TCR (svd-TCR), a non-immunoglobulin antigen binding scaffold, a ligand (e.g., APRIL) or the extracellular domain of a receptor (e.g., PD1, NKG2D, NKp30, NKp44, NKp46 etc.). The chain of a single chain SAR may bind to one antigen or more than one antigen (e.g., two, three, four etc.). The chain of a single chain CD16 SAR may further comprise one or more adaptors (e.g., RZIP, EZIP, NKG2D-YA, NKG2D-FA etc.).

In some embodiments, the CD16 SAR of the disclosure comprises a molecule of the general formula:

AABD(n)-optional CD16 D1 domain-optional CD16 linker domain-optional-CD16 D2 domain, CD16 hinge domain-CD16 transmembrane domain-optional-intracellular costimulatory domain(n)-optional CD16 intracellular signaling domain wherein n is 1 or more. In one embodiment, n is at least 2, for example 2, 3, 4 or 5. The AABD (autonomous antigen binding domain) forms the antigen binding domain and is located at the extracellular side when expressed in a cell.

In an embodiment, the AABD is a fully human vH domain or a humanized vH domain. In an embodiment, the AABD is a fully human single VH (SVH) domain or a humanized SVH domain. An SVH domain, also known as an autonomous vH domain, can bind to a target in the absence of a vL domain. In an embodiment, the AABD is a fully human vHH domain or a humanized vHH domain.

In an embodiment, the AABD is a non-immunoglobulin antigen binding scaffold such as a DARPIN, an affibody, a ZIP domain (e.g., RZIP, EZIP, E4, R4 etc.), an affilin, an adnectin, an affitin, an obodies, a repebody, a fynomer, an alphabody, an avimer, an atrimer, a centyrin, a pronectin, an anticalin, a kunitz domain, an Armadillo repeat protein or a fragment thereof; an extracellular domain of a receptor (e.g., NKG2D), a ligand (e.g., APRIL, Thrombopoietin) and the like.

In some embodiments, the CD16 SAR of the disclosure comprises a molecule of the general formula:

scFv(n)-optional CD16 D1-optional CD16 linker domain-optional-CD16 D2 domain, CD16 hinge domain-CD16 transmembrane domain-optional-intracellular costimulatory domain(n)-optional CD16 intracellular signaling domain, wherein n is 1 or more.

In another embodiment, a costimulatory domain is also incorporated in the CD16 chain(s) of CD16-SAR. Exemplary costimulatory domains include costimulatory domains of 41BB, CD28, OX40 and 2B4 etc. (Table 30; SEQ ID NO (DNA): 1565-1572 and SEQ ID NO (PRT): 3959-3966). Collectively, the above results provide a novel platform for adoptive cellular therapy that overcomes some of the design limitations of current generation CARs and also provide a complementary approach to CARs.

The nucleic acid and amino acid sequences of SARs comprising the entire CD16A in fusion with scFv fragments targeting different antigens are represented by SEQ ID NO (DNA):4851-5039 and SEQ ID NO (PRT): 5151-5339, respectively. The order of the scFv fragments and their target antigens is the same as the order of the scFv and target antigens show in Table 3. The full names of these CD16 based SAR constructs is also provided in Table 36 of the provisional application which is incorporated in its entirety by reference herein. Additional SAR comprising the CD16 full length sequenced attached to different scFv, single domain antibodies, adaptors or scTCR are presented in SEQ ID NO(PRT): 10043-10323. Exemplary SAR comprising the CD16 full length sequence and comprising a vHH fragment or a FHVH fragment attached to an scFv targeting CD19 are represented by SEQ ID NO: 10324-10326. Exemplary SAR comprising the CD16 full length sequence and comprising an adaptor (SEQ ID NO: 10331-32) or a scTCR (SEQ ID NO: 10329-10330) are also provided.

The nucleic acid and amino acid sequences of exemplary SARs comprising the entire CD16A in fusion with vHH and FHVH fragments targeting different antigens are represented by SEQ ID NO (DNA):5040-5108 and SEQ ID NO (PRT): 5340-5408, respectively. The names and target antigens of these SARS are provided in the Table 37 of the provisional application. The nucleic acid and amino acid sequences of exemplary SARs comprising the entire CD16A in fusion with non-immunoglobulin antigen binding domains targeting different antigens are represented by SEQ ID NO (DNA):5110-5121 and SEQ ID NO (PRT): 5410-5421, respectively. The names and target antigens of these SARS are provided in the Table 38 of the provisional application. The nucleic acid and amino acid sequences of exemplary SARs comprising the entire CD16A in fusion with the extracellular antigen binding domains of receptors, adaptors and cytokines are represented by SEQ ID NO (DNA):5123-5129 and SEQ ID NO (PRT): 5423-5429, respectively. The names and target antigens of these SARS are provided in the Table 39 of the provisional application.

The different SARS of this disclosure are modular in design. Therefore, the sequence encoding the CD16A-F158V-FL-v1 (SEQ ID NO: 1415) may be replaced by a sequence encoding different signaling modules (e.g., SEQ ID NO:9635-9740; 9813-9851). Exemplary such modules include CD16-F158V-D2TMCPv1 (SEQ ID NO: 1450), CD16-F158V-Hinge-TM-CP (SEQ ID NO: 1451), NKp30-ECDTMCP-opt1 (SEQ ID NO:1369), NKp30-Hinge-TMCP-opt1 (SEQ ID NO: 1370), NKp44-ECDTMCP-opt1 (SEQ ID NO: 1382), NKp44-Hinge-TM-CP-opt1 (SEQ ID NO: 1383), NKp46-ECDTMCP-opt1 (SEQ ID NO: 1395), NKp46-Linker-Ig1-Hinge-TM-CP-opt1 (SEQ ID NO: 1396), NKp46-Ig1-Hinge-TM-CP-opt1 (SEQ ID NO: 1397), and NKp46-Hinge-TM-CP-opt1 (SEQ ID NO: 1398). The exemplary SARS in which one or more of the CD16A-F158V-FL-v1 modules are replaced with a different signaling modules are represented by SEQ ID NO (PRT): 9860-10042 and SEQ ID NO (DNA): 8768-8950. The names and SEQ ID of the various exemplary constructs are also presented in Table 33 of the provisional application which is incorporated in its entirety by reference herein.

The amino acid sequence of the polypeptides comprising the extracellular, transmembrane and cytosolic domains of different naturally occurring receptors that can be used in the construction of SAR are provided in SEQ ID NO (PRT): 9633-9668. The exemplary SAR are presented in SEQ ID NO:9860-9895. The amino acid sequence of the polypeptides comprising the hinge, transmembrane and cytosolic domains of different naturally occurring receptors that can be used in the construction of SAR are provided in SEQ ID NO (PRT): 9669-9704. The exemplary CD19 SAR comprising a CD19 scFv attached to these polypeptides and a hinge domain of CD28 are presented in SEQ ID NO:9896-9931. The amino acid sequence of the polypeptides comprising the transmembrane and cytosolic domains of different naturally occurring receptors that can be used in the construction of SAR are provided in SEQ ID NO (PRT): 9705-9740. The exemplary CD19 SAR comprising a CD19 scFv attached to these polypeptides and a hinge domain of CD28 are presented in SEQ ID NO:9957-9992. The CD19 scFV domain in any of the above SAR can be replaced with a different antigen binding domain (e.g., scFv, vHH, FHVH, non-immunoglobulin antigen binding domain, scTCR, scv-TCR, ligand binding domain of a receptor, receptor binding domain of a ligand or an adaptor etc.) domain targeting a different antigen to generate novel SARs. Exemplary antigen binding domains are presented in Tables 3-10 of the provisional application. A SAR may also comprise two heterologous antigen binding domains attached to a naturally occurring receptor.

In the one embodiment, the CD16 SAR comprises the entire extracellular domain of CD16 and has the formula: AABD(n)-CD16 D1-CD16 linker domain-CD16 D2 domain-CD16 hinge domain-CD16 transmembrane domain-CD16 intracellular domain, wherein n is 1 or more and where AABD comprises a fully human vH domain or a humanized vHH domain. The CD16 extracellular domain may carry the F158V and S197P mutations. The nucleic acid and amino acid sequences of exemplary CD16 SARs comprising the entire extracellular domain of CD16 and targeting different antigens are provided in SEQ ID NO (DNA):4851-5129 and 8951-9244 and SEQ ID NO (PRT):5151-5429 and 10043-10336 and in the Tables 36-39 of the provisional application. The composition and the order of the antigen binding domains of the SAR constructs of SEQ ID NO: 5151-5429, 8951-9244 is the same as the order of the scFv with SEQ ID NO:2924-3160 shown in Table 3. The constructs with SEQ ID NO:9140-9153 and 9188-9215 target BCMA, constructs with SEQ ID NO: 9216-9222 target PSMA, and those with SEQ ID NO: 9223-9231 target mesothelin. Constructs with SEQ ID NO:9232 and 9234 are bispecific CD16-SAR that target both CD19 and BCMA, while construct with SEQ ID NO: 9233 is bispecific CD16 SAR that targets CD20 and CD19. Constructs with SEQ ID NO: 9237 and 9238 comprise scTCR targeting NY-ESO-1 peptide (SEQ ID NO: 10880) and MAGE-A3 peptide (112-120) (SEQ ID NO:10879) peptides as their target antigen, while SAR construct with SEQ ID NO: 9241 comprises a single variable domain TCR (svd-TCR) targeting MAGE-A3 peptide-270-279 (SEQ ID NO: 10878). Constructs SEQ ID NO: 9239 and 9240 comprise Rzip and EZip adaptors as the antigen binding domains. Finally constructs with SEQ ID NO: 9242-9244 comprise other adaptors.

T cells expressing a single chain CD16-SAR when exposed to a cell expressing the cognate target antigen can activate NFAT signaling, induce IL2 production, promote T cell proliferation, promote T cell activation and exert cytotoxicity. In another exemplary embodiment, NK cells expressing a single chain CD16-SAR when exposed to a cell expressing the cognate target antigen can induce IL2 production, promote NK cell proliferation, promote NK cell activation or exert cytotoxicity. In another exemplary embodiment, monocytes/macrophages expressing a single chain CD16-SAR when exposed to a cell expressing the cognate target antigen can induce phagocytosis of the target cells. In another exemplary embodiment, granulocytes (e.g., neutrophils) expressing a single chain CD16-SAR when exposed to a cell expressing the cognate target antigen can induce phagocytosis of the target cells.

In certain embodiments, the disclosure provides a novel platform of synthetic antigen receptors, designated CD16-SARs, containing two chains wherein each chain comprises the partial or the entire sequence of CD16 or a variant thereof.

In an embodiment, the disclosure provides a double chain CD16 SARs where each chain comprises a partial or entire region of CD16 extracellular domain. Exemplary CD16 extracellular domain sequences that can be used in the construction of double chain CD16-SARs of the disclosure are provided in SEQ ID NO (DNA): 1496-1509 and SEQ ID NO (PRT): 3890-3903. In an embodiment, the disclosure provides double chain CD16 SARs where each chain comprises a partial or entire region of CD16 hinge domain. Exemplary CD16 hinge domain sequences that can be used in the construction of double chain CD16-SARs of the disclosure are provided in SEQ ID NO (DNA): 1545-1547 and SEQ ID NO (PRT): 3939-3941. In an embodiment, the disclosure provides a double chain CD16 SAR where each chain comprises partial or entire region of CD16 transmembrane domain. Exemplary CD16 transmembrane sequences that can be used in the construction of double chain CD16-SARs of the disclosure are provided in SEQ ID NO (DNA): 1528-1530 and SEQ ID NO (PRT): 3922-3924. In an embodiment, the disclosure provides a double chain CD16 SAR where each chain comprises a partial or entire region of CD16 cytosolic domain. Exemplary CD16 transmembrane sequences that can be used in the construction of CD16-SARs of the disclosure are provided in SEQ ID NO (DNA): 1556-1558 and SEQ ID NO (PRT): 3950-3952.

The disclosure provides that the vL fragment of an antibody can be joined to one of the two CD16 chains and the vH fragment can be joined to the other CD16 chain. When the two such chains (e.g., vL-CD16 and vH-CD16) are co-expressed in the same cell, the vL and vH fragments can bind their cognate antigen and transmit a cell signal. In an exemplary embodiment, T cells expressing such CD16-SAR when exposed to a cell expressing the cognate target antigen can activate NFAT signaling, induce IL2 production, promote T cell proliferation, promote T cell activation and exert cytotoxicity. In another exemplary embodiment, NK cells expressing such CD16-SAR when exposed to a cell expressing the cognate target antigen can induce IL2 production, promote NK cell proliferation, promote NK cell activation or exert cytotoxicity. In another exemplary embodiment, monocytes/macrophages expressing such CD16-SAR when exposed to a cell expressing the cognate target antigen can induce phagocytosis of the target cells. In another exemplary embodiment, monocytes/macrophages expressing a single chain CD16-SAR when exposed to a cell expressing the cognate target antigen can induce phagocytosis of the target cells. In another exemplary embodiment, granulocytes (e.g., neutrophils) expressing a single chain CD16-SAR when exposed to a cell expressing the cognate target antigen can induce phagocytosis of the target cells.

The expression and activity of the double chain CD16-SAR can be further increased by incorporation of a linker between the vL/vH and the CD16 fragments. In particular, the IgCL (SEQ ID NO (DNA): 1142 and SEQ ID NO (PRT): 3536) and IgCH domains (SEQ ID NO (DNA): 1143-1157 and SEQ ID NO (PRT): 3537-3551) derived from antibodies serve as useful linkers between the vL/vH and CD16 fragments. Additional Ig like domains are known in the art (e.g., Table 13; SEQ ID NO (DNA):1168-1175 and SEQ ID NO (PRT):3562-3569) and can serve as useful linkers in alternate embodiment of the disclosure.

In an embodiment, each chain of the double chain CD16 SAR comprises the CD16 extracellular domain comprising both immunoglobulin like domains (i.e., D1 and D2) that is attached via the CD16 hinge domain to CD16 transmembrane domain and to CD16 cytosolic domain. An exemplary such double chain CD16 SAR targeting CD20 and BCMA is represented by the SAR CD8SP-CD20-VHH-2HC2D6-USC1-xho-IgCL-Bam-CD16-F158V-FL-TMCP-v1-F-P2A-SP-Apa-BCMA917-vHH-E59D-Mlu-IgG1-CH1-Kpn-CD16-F158V-S197P-FL-TMCP-v3-F-F2A-PAC (SEQ ID NO(DNA): 1633 and SEQ ID NO (PRT): 4027). In this SAR, a CD20 vHH domain is attached to one CD16 chain via an IgCL linker and a BCMA vHH is attached to a second CD16 chain via an IgG1-CH1 linker. The two chains of this double chain CD16 SAR are expressed from a single vector with an intervening P2A cleavable linker. This SAR construct also expresses a puromycin resistance cassette (PAC), which is optional. Another exemplary double chain CD16 SAR targeting CD19 is represented by CD8SP-hu-mROO5-1-vL-xho-IgCL-Bam-CD16-F158V-FL-TMCP-v1-F-P2A-SP-hu-mROO5-1-vH-Mlu-IgG1-CH1-Kpn-CD16-F158V-S197P-FL-TMCP-v3-F-F2A-K13-opt (SEQ ID NO(DNA): 1628 and SEQ ID NO (PRT): 4022). In this SAR, a hu-mROO5-1 vL domain is attached to one CD16 chain via an IgCL linker and a hu-mROO5-1 vH domain is attached to a second CD16 chain via an IgG1-CH1 linker. The hu-mROO5-1 vL and vH fragments join to form a Fv that can bind to human CD19. The two chains of this double chain CD16 SAR are expressed from a single vector with an intervening P2A cleavable linker. This SAR construct also expresses an accessory module comprising codon optimized vFLIP K13 module from human herpesvirus 8, which is optional. The double chain CD16 SAR represented by SEQ ID NO (DNA): 1629 and SEQ ID NO (PRT): 4023 is similar to the SAR construct represented by SEQ ID NO (DNA): 1628 and SEQ ID NO (PRT): 4022 with the exception that the K13 module is replaced by MC159 module from molluscum contagiosum virus. The double chain CD16 SAR represented by SEQ ID NO (DNA): 1630 and SEQ ID NO (PRT): 4024 is similar to the SAR construct represented by SEQ ID NO (DNA): 1628 and SEQ ID NO (PRT): 4022 with the exception that the K13 module is replaced by the puromycin resistance gene. The double chain CD16 SAR represented by SEQ ID NO (DNA): 1625 and SEQ ID NO (PRT): 4020 is similar to the SAR construct represented by SEQ ID NO (DNA): 1630 and SEQ ID NO (PRT): 4024 with the exception that the IgCL and IgG1-CH1 linker domains are missing and the hu-mROO5-1 vL and vH fragments are attached to the two CD16 chains directly. The double chain CD16 SAR represented by SEQ ID NO (DNA): 1631 and SEQ ID NO (PRT): 4025 is similar to the SAR construct represented by SEQ ID NO (DNA): 1630 and SEQ ID NO (PRT): 4024 with the exception that a vHH domain targeting human CD20 is attached to the amino-terminus of hu-mROO5-1 vL region via a short Gly4Serx2 linker (SEQ ID NO (DNA): 1024). This construct can target both CD19 and CD20. The double chain CD16 SAR represented by SEQ ID NO (DNA): 1632 and SEQ ID NO (PRT): 4026 is similar to the SAR construct represented by SEQ ID NO (DNA): 1631 and SEQ ID NO (PRT): 4025 with the exception that a vHH domain targeting human BCMA is attached to the amino-terminus of hu-mROO5-1 vH region via a short G4Sx3linker (SEQ ID NO (DNA): 40). This construct can target CD19, CD20 and BCMA. The double chain CD16 SAR represented by SEQ ID NO (DNA): 1634 and SEQ ID NO (PRT): 4028 is similar to the SAR construct represented by SEQ ID NO(DNA): 1630 and SEQ ID NO (PRT): 4024 with the exception that the IgCL and IgG1-CH1 linker domains are replaced by TCRb-ECD (TCRb-wt-opt-8ECD; SEQ ID NO: 1166) and TCRa-ECD (TCRa-Ig-Like-C1-Domain-6MD; SEQ ID NO: 1168) linker domains, respectively. The double chain CD16 SAR represented by SEQ ID NO (DNA): 1635 and SEQ ID NO (PRT): 4029 is similar to the SAR construct represented by SEQ ID NO(DNA): 1631 and SEQ ID NO (PRT): 4025 with the exception that the IgCL and IgG1-CH1 linker domains are replaced by TCRb-ECD (TCRb-wt-opt-8ECD; SEQ ID NO: 1166) and TCRa-ECD (TCRa-Ig-Like-C1-Domain-6MD; SEQ ID NO: 1168) linker domains, respectively. The double chain CD16 SAR represented by SEQ ID NO (DNA): 1636 and SEQ ID NO (PRT): 4030 is similar to the SAR construct represented by SEQ ID NO(DNA): 1632 and SEQ ID NO (PRT): 4026 with the exception that the IgCL and IgG1-CH1 linker domains are replaced by TCRb-ECD (TCRb-wt-opt-8ECD; SEQ ID NO: 1166) and TCRa-ECD (TCRa-Ig-Like-C1-Domain-6MD; SEQ ID NO: 1168) linker domains, respectively. The double chain CD16 SAR represented by SEQ ID NO (DNA): 1637 and SEQ ID NO (PRT): 4031 is similar to the SAR construct represented by SEQ ID NO(DNA): 1633 and SEQ ID NO (PRT): 4027 with the exception that the IgCL and IgG1-CH1 linker domains are replaced by TCRb-ECD (TCRb-wt-opt-8ECD; SEQ ID NO: 1166) and TCRa-ECD (TCRa-Ig-Like-C1-Domain-6MD; SEQ ID NO: 1168) linker domains, respectively.

In an embodiment, the disclosure provides a double chain CD16 SAR where each chain comprises a partial or entire region of CD16. In an embodiment, the disclosure provides a double chain CD16 SARs where each chain comprises a partial or entire region of CD16 extracellular domain. In an embodiment, the disclosure provides a double chain CD16 SARs where each chain comprises a partial or entire region of CD16 D1 domain. In an embodiment, the disclosure provides a double chain CD16 SARs where each chain comprises a partial or entire region of CD16 D2 domain. In an embodiment, the disclosure provides a double chain CD16 SARs where each chain comprises a partial or entire region of CD16 hinge domain. In an embodiment, the disclosure provides a double chain CD16 SARs where each chain comprises a partial or entire region of a CD16 transmembrane domain. In an embodiment, the disclosure provides a double chain CD16 SARs where each chain comprises a partial or entire region of a CD16 cytosolic domain.

In an embodiment, each chain of the double chain CD16 SAR comprises the CD16 extracellular domain comprising both immunoglobulin like domains (i.e., D1 and D2) that is attached via the CD16 hinge domain to CD16 transmembrane domain and CD16 cytosolic domain. In an embodiment, each chain of a double chain CD16 SAR also retains the ability to bind to the Fc region of an antibody, an antibody fragment or bispecific/tri-specific engager and mediate antibody dependent cytotoxicity. In an embodiment, each chain of the double chain CD16 SAR comprises the partial CD16 extracellular domain comprising the 2nd immunoglobulin like domains (i.e., D2) that is attached via CD16 hinge domain to CD16 transmembrane domain and CD16 cytosolic domain. In an embodiment, each chain of such double chain CD16 SAR lacks the ability to bind to the Fc portion of an antibody or an antibody fragment as it contains only the D2 domain of CD16 and lacks the D1 domain. In an embodiment, each chain of the double chain CD16 SAR comprises the partial or entire CD16 hinge domain that is attached to CD16 transmembrane domain and CD16 cytosolic domain. In an embodiment, each chain of such double chain CD16 SAR lacks the ability to bind to the Fc portion of an antibody or an antibody fragment as it lacks both the D1 and D2 domains.

In an embodiment, at least one chain of the double chain CD16 SAR comprises, an AABD (e.g., a vHH, FHVH, chVH, centyrin, affibody etc.) that is inserted between the D2 domain and the hinge domain of CD16 with optional intervening linkers (e.g., Glycine-Serine linker). In an exemplary embodiment, the different domains of such a chain comprising a double chain CD16 SAR from amino to carboxy-terminal include a N-terminal signal peptide, CD16-D1 domain, CD16-D2 domain, optional linker, AABD (e.g., vHH, FHVH, centyrin, affibody etc.), optional linker, CD16-hinge domain, CD16-transmembrane domain and CD16-cytosolic domain.

It is to be understood that the different CD16 domains (i.e., extracellular, D1, D2, hinge, transmembrane and cytosolic etc.) that may be used in the construction of the double chain CD16 SAR may comprise their entire sequence or a deletion mutant or a variant as long as it retains the functional property of that domain. In an embodiment, the antigen binding domain of one or both chains of the double chain CD16 SAR comprises a scFv, a vL, vH, Fv, vHH, FHVH, a single domain antibody, a non-immunoglobulin antigen binding scaffold, a ligand or the extracellular domain of a receptor.

In an embodiment, both chains of a double chain CD16 SAR comprise an antigen binding domain. In an embodiment, only one of the chains of a double chain CD16 SAR comprise an antigen binding domain. In an embodiment, one of the chains of a double chain CD16 SAR comprise a non-natural antigen binding domain (e.g., a scFv, a vL, vH, Fv, vHH, FHVH, a single domain antibody, a non-immunoglobulin antigen binding scaffold, a ligand or the extracellular domain of a receptor) and the second chain binds to Fc portion of an antibody or antibody fragment or a bispecific/trispecific engager via the CD16 extracellular domain.

In an embodiment, one chain of a double chain CD16 SAR comprises an antigen binding domain consisting of a vL domain and the second chain of the double chain CD16 SAR comprises an antigen binding domain consisting of a vH domain. In an embodiment, both chains of a double chain CD16 SAR comprise an antigen binding domain of the same class (i.e., scFv, vHH, FHVH, a single domain antibody, a non-immunoglobulin antigen binding scaffold, a ligand or a receptor etc.). In an embodiment, each chain of a double chain CD16 SAR comprise a vHH domain. In an embodiment, each chain of a double chain CD16 SAR comprise a FHVH domain. In an embodiment, both chains of a double chain CD16 SAR comprise an antigen binding domain of different classes (i.e., scFv, vHH, FHVH, a single domain antibody, a non-immunoglobulin antigen binding scaffold, a ligand or a receptor etc.). In an exemplary embodiment, one chain of a double chain CD16 SAR comprises an antigen binding domain derived from vHH domain while the second chain comprises an antigen binding domain derived from a FHVH domain.

The two chains of a double chain CD16 SAR may target the same antigen (e.g., CD19) or different antigens (e.g., CD19 and CD20). The two chains of a double chain CD16 SAR may target two different epitopes of a single antigen (e.g., CD19) or two different antigens (e.g., CD19 and CD20). Each chain of a double chain SAR may bind to one antigen or more than one antigen (e.g., two, three, four etc.). Each chain of a double chain CD16 SAR may further comprise adaptors (e.g., RZIP, EZIP, NKG2D-YA, NKG2D-FA etc.).

In another embodiment, a costimulatory domain is also incorporated in one or both of the CD16 chain(s) of a double chain CD16-SAR. Exemplary costimulatory domains include costimulatory domains of 41BB, CD28, OX40 and 2B4 etc. (Table 30; SEQ ID NO (DNA): 1565-1572 and SEQ ID NO (PRT): 3959-3966). Collectively, the above results provide a novel platform for adoptive cellular therapy that overcomes some of the design limitations of CAR and also provide a complementary approach to SARs.

The two chains of CD16A-SARs described herein may be encoded by a single polynucleotide chain and translated into a single polypeptide chain, which is subsequently cleaved into different proteins. The two chains of CD16A-SARs described herein may be expressed using two distinct promoters and encoded by two separate polynucleotide chains. The two chains of CD16A-SARs described herein may be encoded by a single vector. The two chains of CD16A-SARs described herein may be encoded by a two different vector. The nucleic acid molecule encoding a CD16-SAR can comprise one or more leader sequences (also known as a signal peptide). In one embodiment, each functional unit (e.g., an antigen binding domain joined to a CD3z chain plus Furine-SGSG-cleavable linker) of a CD16A-SAR can be preceded by a leader sequence which directs the CD16A-SAR to the cell surface as a type I transmembrane protein. In one embodiment, the antigen-binding domain of CD16-SAR is extracellular-facing. In some embodiments, the leader sequence comprises the nucleic acid sequence of any of SEQ ID NO: 31 to 34 and amino acid sequences of SEQ ID NO: 2425 to 2428. In some embodiments, short nucleic acid sequences (3-9 nucleic acids) comprising restriction enzyme sites are located between the different subunits of a CD16A-SAR, e.g., between a signal sequence and the antigen binding domain of the CD16-SAR or between the antigen binding and the CD16 chain.

The different SARS of this disclosure are modular in design. Therefore, the sequence encoding the CD16A-F158V-FL-v1 (SEQ ID NO: 1415) and CD16-F158V-S197P-FL-TMCP-v3 (SEQ ID NO:1417) may be replaced by a sequence encoding different signaling modules (Table 25). Exemplary such modules include CD16-F158V-D2TMCPv1 (SEQ ID NO: 1450), CD16-F158V-Hinge-TM-CP (SEQ ID NO: 1451), NKp30-ECDTMCP-opt1 (SEQ ID NO:1369), NKp30-Hinge-TMCP-opt1 (SEQ ID NO: 1370), NKp44-ECDTMCP-opt1 (SEQ ID NO: 1382), NKp44-Hinge-TM-CP-opt1 (SEQ ID NO: 1383), NKp46-ECDTMCP-opt1 (SEQ ID NO: 1395), NKp46-Linker-Ig1-Hinge-TM-CP-opt1 (SEQ ID NO: 1396), NKp46-Ig1-Hinge-TM-CP-opt1 (SEQ ID NO: 1397), and NKp46-Hinge-TM-CP-opt1 (SEQ ID NO: 1398), 41BB-ECDTMCP-opt1 (SEQ ID NO: 1573), CD28-ECDTMCP-opt1 (SEQ ID NO: 1575), OX40-ECDTMCP-opt1 (SEQ ID NO: 1577), 2B4-ECDTMCP-opt1 (SEQ ID NO: 1579), CD32-ECDTMCP-opt1 (SEQ ID NO: 1581) and CD64-ECDTMCP-opt1 (SEQ ID NO: 1583). The SEQ ID NOs of exemplary SARS in which one or more of the CD16A-F158V-FL-v1 and CD16-F158V-S197P-FL-TMCP-v3 modules are replaced with a different signaling modules are presented in Table 33 of provisional application.

In certain embodiments, the disclosure provides a novel platform of synthetic antigen receptors, designated CD16-SARs, containing two chains, one of which incorporates the partial or entire region of CD16.

In alternate embodiment, the disclosure provides a double chain CD16 SARs where one of the chains comprises a partial or entire region of CD16 extracellular domain. Exemplary CD16 extracellular domain sequences that can be used in the construction of double chain CD16-SARs of the disclosure are provided in SEQ ID NO (DNA): 1496-1509 and SEQ ID NO (PRT): 3890-3903. In an embodiment, the disclosure provides double chain CD16 SARs where one of the chains comprises a partial or entire region of CD16 hinge domain. Exemplary CD16 hinge domain sequences that can be used in the construction of double chain CD16-SARs of the disclosure are provided in SEQ ID NO (DNA): 1545-1547 and SEQ ID NO (PRT): 3939-3941. In an embodiment, the disclosure provides a double chain CD16 SAR where one of the chains comprises partial or entire region of CD16 transmembrane domain. Exemplary CD16 transmembrane sequences that can be used in the construction of double chain CD16-SARs of the disclosure are provided in SEQ ID NO (DNA): 1528-1530 and SEQ ID NO (PRT): 3922-3924. In an embodiment, the disclosure provides a double chain CD16 SAR where one of the chains comprises a partial or entire region of CD16 cytosolic domain. Exemplary CD16 transmembrane sequences that can be used in the construction of CD16-SARs of the disclosure are provided in SEQ ID NO (DNA): 1556-1558 and SEQ ID NO (PRT): 3950-3952.

The disclosure provides that the vL fragment of an antibody can be joined to a CD16 chain and the vH fragment can be joined to the another signaling chain, such as CD3z, FcRγ. NKp30, NKp44, NKp46, TCRα constant chain, TCRβ constant chain, TCRγ constant chain or TCRδ constant chain etc. Alternatively, the disclosure provides that the vH fragment of an antibody can be joined to a CD16 chain and the vL fragment can be joined to the another signaling chain, such as CD3z, FcRγ, NKp30, NKp44, NKp46, TCRα constant chain, TCRβ constant chain, TCRγ constant chain or TCRδ constant chain etc. When the two such chains (e.g., vL-CD16 and vH-CD3z) are co-expressed in the same cell, the vL and vH fragments can bind their cognate antigen and transmit a T cell signal. In particular, T cells expressing such CD16-hererodimeric SAR when exposed to a cell line expressing the cognate target antigen can activate NFAT signaling, induce IL2 production, promote T cell proliferation, promote T cell activation and exert cytotoxicity. In another exemplary embodiment, NK cells expressing such CD16-SAR when exposed to a cell line expressing the cognate target antigen can induce IL2 production, promote NK cell proliferation, promote NK cell activation or exert cytotoxicity. The expression and activity of the CD16-heterodimeic SAR can be further increased by incorporation of a linker between the vL/vH and the CD16 and the other signaling chains (e.g., CD3z, FcRγ, NKp30, NKp44, NKp46 etc.). In particular, the IgCL (SEQ ID NO (DNA): 1142 and SEQ ID NO (PRT): 3536) and IgCH domains (SEQ ID NO (DNA): 1143-1157 and SEQ ID NO (PRT): 3537-3551) derived from antibodies serve as useful linkers between the vL/vH and CD16 fragments. Additional Ig like domains are known in the art (e.g., Table 13; SEQ ID NO (DNA):1168-1175 and SEQ ID NO (PRT):3562-3569) and can serve as useful linkers in alternate embodiment of the disclosure.

The different SARS of this disclosure are modular in design. Therefore, the sequence encoding the CD16A-F158V-FL-v1 (SEQ ID NO: 1415) may be replaced by a sequence encoding different signaling modules shown in the Table 25 of provisional application. Exemplary such modules include CD16-F158V-D2TMCPv1 (SEQ ID NO: 1450), CD16-F158V-Hinge-TM-CP (SEQ ID NO: 1451), NKp30-ECDTMCP-opt1 (SEQ ID NO: 1369), NKp30-Hinge-TMCP-opt1 (SEQ ID NO: 1370), NKp44-ECDTMCP-opt1 (SEQ ID NO: 1382), NKp44-Hinge-TM-CP-opt1 (SEQ ID NO: 1383), NKp46-ECDTMCP-opt1 (SEQ ID NO: 1395), NKp46-Linker-Ig1-Hinge-TM-CP-opt1 (SEQ ID NO: 1396), NKp46-Ig1-Hinge-TM-CP-opt1 (SEQ ID NO: 1397), NKp46-Hinge-TM-CP-opt1 (SEQ ID NO: 1398), 41BB-ECDTMCP-opt1 (SEQ ID NO: 1573), CD28-ECDTMCP-opt1 (SEQ ID NO: 1575), OX40-ECDTMCP-opt1 (SEQ ID NO: 1577), 2B4-ECDTMCP-opt1 (SEQ ID NO: 1579), CD32-ECDTMCP-opt1 (SEQ ID NO: 1581) and CD64-ECDTMCP-opt1 (SEQ ID NO: 1583). The SEQ ID NOs of exemplary SARS in which one or more of the CD16A-F158V-FL-v1 modules are replaced with a different signaling modules are presented in Table 33.

Also provided herein are clonal iPSCs genetically engineered to comprise, among other editing as contemplated and described herein, a CD16 SAR. In an embodiment, the CD16 SAR is a high affinity CD16 SAR or a high-affinity non-cleavable CD16 SAR (hnCD16-SAR). The genetically engineered iPSCs are capable of differentiating into effector cells comprising the CD16-SAR (e.g., high affinity CD16 SAR or hnCD16-SAR) introduced to the iPSCs. In some embodiments, the derived effector cells comprising CD16-SAR are NK cells. In some embodiments, the derived effector cells comprising CD16-SAR are T cells. In an embodiment, the CD16-SAR (e.g., high affinity CD16 SAR or hnCD16-SAR) expressed in iPSC or derivative cells thereof binds to not only ADCC antibodies or fragments thereof, but also to bi-, tri-, or multi-specific engagers or binders that recognize the CD16 or CD64 extracellular binding domains of said CD16 SAR. As such, the present application provides a derivative effector cell or a cell population thereof, preloaded with one or more pre-selected ADCC antibody through binding with the extracellular domain of the CD16-SAR expressed on the derivative effector cell, in an amount sufficient for therapeutic use in a treatment of a condition, a disease, wherein said CD16-SAR comprises an extracellular binding domain of CD64, or of CD16 having FI76V and S197P. In an embodiment, the antigen binding domain of the CD16-SAR comprises an AABD, a scFv, Fv, extracellular domain of a receptor, ligand, or another non-immunoglobulin antigen binding module. In an embodiment, the CD16-SAR comprises an antigen binding domain attached to or near the N-terminus of the Fc binding domain of CD16 or CD64. In an embodiment, the CD16-SAR further comprises an antigen binding domain (e.g., AABD, e.g., FHVH, chVH, aVH, vHH, Darpin, centyrin, affibody etc.) attached to or near the N-terminus of the Fc binding domain of CD16 or CD64.

In some other embodiments, the native CD16 transmembrane- and/or the intracellular-domain of a CD16-SAR (e.g., high affinity CD16 SAR or hnCD16-SAR) is further modified or replaced, such that a chimeric Fc-SAR (CFc-SAR) is produced to comprise a non-native transmembrane domain, a non-native stimulatory domain and/or a non-native signaling domain. The term “non-native” or “non-natural” used herein means that the transmembrane, stimulatory or signaling domain are derived from a different receptor other than the receptor which provides the extracellular domain. In the illustration here, the CFc-SAR based on CD16 or variants thereof does not have a transmembrane, stimulatory or signaling domain that is derived from CD16. In some embodiments, the exogenous CD16 based CFc-SAR comprises a non-native transmembrane domain derived from CD3D, CD3ε, CD3G, CD3z, CD4, CD8, CD8a, CD8b, CD27, CD28, CD40, CD84, CD166, 4-1BB, 0X40, ICOS, ICAM-1, CTLA-4, PD-1, LAG-3, 2B4, BTLA, CD16, IL-7, IL12, IL15, KIR2DL4, KIR2DSI, MKp30, MKp44, NKp46, NKG2C, NKG2D, T cell receptor polypeptide. In some embodiments, the CD16 based CFc-SAR comprises a non-native stimulatory/inhibitory domain derived from CD27, CD28, 4-1BB, 0X40, ICOS, PD-1, LAG-3, 2B4, BTLA, DAP10, DAP 12, CTLA-4, or NKG2D polypeptide. In some embodiments, the exogenous CD16 based CFc-SAR comprises a non-native signaling domain derived from CD3z, 2B4, DAP 10, DAP 12, DNAM1, CD137 (4 IBB), IL21, IL7, IL12, IL15, NKp30, NKp44, NKp46, NKG2C, or NKG2D polypeptide. In one embodiment of CD16-SAR, the provided chimeric receptor comprises a transmembrane domain and a signaling domain both derived from one of IL7, IL12, IL15, NKp30, NKp44, NKp46, NKG2C, and NKG2D polypeptide. One particular embodiment of the CD16 based CFc-SAR comprises a transmembrane domain of NKG2D, a stimulatory domain of 2B4, and a signaling domain of CD3z; wherein the extracellular domain of the CD16 is derived from a full length or partial sequence of the extracellular domain of CD64 or CD16, wherein the extracellular domain of CD16 comprises F176V (or 158V) and S197P (or S197R).

Another embodiment of the CD16 based chimeric Fc-SAR comprises a transmembrane domain and a signaling domain of CD3z; wherein the extracellular domain of the CD16 is derived from a full length or partial sequence of the extracellular domain of CD64 or CD16, wherein the extracellular domain of CD16 comprises FI 76V and S197P. In an embodiment, the antigen binding domain of the CD16-chimeric Fc SAR comprises an AABD (e.g., FHVH, vHH etc.), a scFv, Fv, ligand, extracellular domain of a receptor, or another non-immunoglobulin antigen binding module. In an embodiment, the CD16-chimeric Fc SAR further comprises an antigen binding domain attached to or near the N-terminus of the Fc binding domain of CD16 or CD64. In an embodiment, the hnCD16-chimeric Fc SAR further comprises an antigen binding domain (e.g., AABD, e.g., FHVH, chVH, aVH, vHH, Darpin, centyrin, affibody etc.) attached to or near the N-terminus of the Fc binding domain of CD16 or CD64.

The various embodiments of CD16 based chimeric Fc SAR as described above are capable of binding to the Fc region of an antibody or fragment thereof; or to the Fc region of a bi-, tri-, or multi-specific engager or binder. In addition, the CD16 based chimeric Fc SAR are capable of binding to an antigen specified by their antigen binding domain (i.e., AABD, scFv, Fv, etc.). Thus, a CD16 based chimeric Fc SAR with an antigen binding domain based on BCMA-FHVH may bind to the Fc region of an antibody while also having the capability of binding BCMA. Upon binding, the stimulatory and/or signaling domains of the CD16-CFc SAR enable the activation and cytokine secretion of the effector cells, and the killing of the tumor cells targeted by the antibody or their antigen binding domain (e.g., AABD, scFv, Fv etc.), or said bi-, tri-, or multi-specific engager or binder having a tumor antigen binding component as well as the Fc region. Without being limited by theory, through the non-native transmembrane, stimulatory and/or signaling domains, or through an engager binding to the ectodomain, of the CD16 based chimeric Fc receptor, the CFc-SAR could contribute to effector cells' killing ability while increasing the effector cells' proliferation and/or expansion potential. The antibody and the engager can bring tumor cells expressing the antigen and the effector cells expressing the CFc-SAR into a close proximity, which also contributes to the enhanced killing of the tumor cells. Exemplary tumor antigen for bi-, tri-, multi-specific engager or binders include, but are not limited to, B7H3, BCMA, CD10, CD19, CD20, CD22, CD24, CD30, CD33, CD34, CD3ε, CD44, CD79a, CD79b, CD123, CD138, CD179b, CEA, CLEC 12A, CS-1, DLL3, EGFR, EGFRvIII, EPCAM, FLT-3, FOLR1, FOLR3, GD2, gpA33, HER2, HM1.24, LGR5, MSLN, MCSP, MICA/B, PSMA, PAMA, P-cadherin, and ROR1. Some non-limiting exemplary bi-, tri-, multi-specific engager or binders suitable for engaging effector cells expressing the CD16 based CFc-SAR in attacking tumor cells include CD16 (or CD64)-CD30, CD16 (or CD64)-BCMA, CD16 (or CD64)-IL15-EPCAM, and CD 16 (or CD64)-IL15-CD33.

Unlike the endogenous CD16 receptor expressed by primary NK cells which gets cleaved from the cellular surface following NK cell activation, the non-cleavable versions of CD16-SAR (e.g. hnCD16-SAR) in derivative NK avoids CD16 shedding and maintains constant expression. In derivative NK cell, non-cleavable CD16-SAR increases expression of TNFa and CD107a indicative of improved cell functionality. Non-cleavable CD16 also enhances the antibody-dependent cell-mediated cytotoxicity (ADCC), and the engagement of bi-, tri-, or multi-specific engagers. ADCC is a mechanism of NK cell mediated lysis through the binding of CD16 to antibody-coated target cells. The additional high affinity characteristics of the introduced hnCD16-SAR in derived NK cell also enables in vitro loading of ADCC antibody to the NK cell through CD16 before administering the cell to a subject in need of a cell therapy. As provided, the hnCD16-SAR may comprise F176V (or 158V) and S197P (or S197R). As disclosed, the present application also provides a derivative NK cell or a cell population thereof, preloaded with one or more pre-selected ADCC antibody in an amount sufficient for therapeutic use in a treatment of a condition, a disease, or an infection.

In an embodiment, the CD16-SAR of the disclosure comprise the wild-type CD16 sequence attached at or near its N-terminus to an antigen binding domain (e.g., AABD, scFv, Fv etc.). Thus, the CD16-SAR of the disclosure may comprise the hnCD16 or wild-type CD16 coding regions.

In an embodiment, the CD16-SAR of the disclosure comprise the Fc binding region of CD32 or CD64 fused in frame to the transmembrane and intracellular domain of CD16 or variant thereof. In an exemplary embodiment, the order of different modules in such a CD16 SAR may comprise from NH2 to C-terminus the following: Antigen binding domain(n)-CD32-Fc binding domain-CD16 transmembrane domain-CD16-intracellualr domain; where n=1, 2, 3, or more.

In an exemplary embodiment, the order of different modules in such a CD16 SAR may comprise from NH2 to C-terminus the following: where n=1, 2, 3, or more. Antigen binding domain(n)-CD64-Fc binding domain-CD16 transmembrane domain-CD16-intracellualr domain

An exemplary CD20-targeted SAR construct containing a CD20 vHH domain fused to the extracellular domain of CD64 and transmembrane and intracellular domains of CD16 is represented by SEQ ID NO (DNA): 2328 and SEQ ID NO (PRT): 4722. Additional SAR construct targeting other antigens can be generated by replacing the CD20 vHH domain with antigen binding domains (e.g., scFv, vHH, FHVH, Centyrin etc.) targeting different antigens.

Unlike primary NK cells, mature T cells from a primary source (i.e., natural/native sources such as peripheral blood, umbilical cord blood, or other donor tissues) do not express CD16. It was unexpected that mature T cells expressing the exogeneous CD16-SAR construct show cell surface expression of the CD16 SAR and are capable of transmitting a cell signal (e.g., NFAT signaling) when exposed to the target antigen expressing cells.

It was also unexpected that iPSC comprising an expressed exogenous CD16-SAR did not impair the T cell developmental biology and was able to differentiate into functional derivative T cells that not only express the exogenous CD16-SAR, but also are capable of carrying out function through an acquired ADCC mechanism. This acquired ADCC in the derivative T cell can additionally be used as an approach for dual targeting and/or to rescue antigen escape often occurred with CAR-T cell therapy, where the tumor relapses with reduced or lost CAR-T targeted antigen expression or expression of a mutated antigen to avoid recognition by the CAR (chimerical antigen receptor). When said derivative T cell comprises acquired ADCC through exogenous CD16-SAR expression, and when an antibody targets a different tumor antigen from the one targeted by the SAR, the antibody can be used to rescue SAR-T antigen escape and reduce or prevent relapse or recurrence of the targeted tumor often seen in CAR-T treatment. Such a strategy to reduce and/or prevent antigen escape while achieving dual targeting is equally applicable to NK cells expressing one or more SARs.

As such, the disclosure provides a derivative T cell comprising an exogenous CD16-SAR. In some embodiment, the CD16-SAR comprise the wild-type sequence of CD16. In some embodiments, the hnCD16 comprised in the derivative T cell comprises F176V (158V) and S197R (or S197P). In some other embodiments, the hnCD16 comprised in the derivative T cell comprises a full or partial ectodomain originated from CD64 or may further comprises at least one of non-native transmembrane domain, stimulatory domain and signaling domain. As explained, such derivative T cells have an acquired mechanism to target tumors with a monoclonal antibody meditated by ADCC to enhance therapeutic effect of the antibody. As disclosed, the present application also provides a derivative T cell or a cell population thereof, preloaded with one or more pre-selected ADCC antibody in an amount sufficient for therapeutic use in a treatment of a condition, a disease, or an infection.

In addition to primary NK and T cells, the CD16 SARs of the disclosure can be expressed in immortalized cell lines. Exemplary immortalized cell lines suitable for expression of the CD16 SARs of the disclosure include NK92 and NK92MI cell lines. Additionally, CD16 SARs of the disclosure can be expressed in pluripotent hematopoietic stem cells (e.g., CD34+ stem cells), which can be differentiated to generate CD16 SAR expressing blood cells belonging to different lineages.

In certain embodiments, the disclosure provides a novel platform of synthetic antigen receptors, designated NKp30-SARs, containing the entire or partial sequence of a NKp30 chain.

The nucleic acid sequences of the NKp30 chains that can be used in the construction of NKp30 SARs are provided in SEQ ID NO: 1395 to 1414 (Table 25 of provisional application). The corresponding amino acid sequences are provided in SEQ ID NO: 3789 to 3808, respectively.

In an embodiment, the disclosure provides a single chain NKp30 SAR comprising a partial or entire region of NKp30. In alternate embodiment, the disclosure provides a single chain NKp30 SARs comprising a partial or entire region of NKp30 extracellular domain. In an embodiment, the disclosure provides NKp30 SARs comprising a partial or entire region of NKp30 hinge domain. In an embodiment, the disclosure provides a NKp30 SAR comprising partial or entire region of NKp30 transmembrane domain. In an embodiment, the disclosure provides a NKp30 SAR comprising a partial or entire region of NKp30 cytosolic domain. In an embodiment, a NKp30 SAR comprises the NKp30 Ig domain that is attached via NKp30 hinge domain to NKp30 transmembrane domain and NKp30 cytosolic domain. In an embodiment, a NKp30 SAR comprises the NKp30 hinge domain that is attached to NKp30 transmembrane domain and NKp30 cytosolic domain.

It is to be understood that the different NKp30 domains (i.e., extracellular, Ig domain, hinge, transmembrane and cytosolic) that may be used in the construction of the SAR may comprise their entire sequence or a deletion mutant or a variant as long as the domain retains at least some of its functional property.

In an embodiment, the antigen binding domain of the NKp30 SAR comprises a scFv, a vL, vH, Fv, vHH, FHVH, a single domain antibody, a non-immunoglobulin antigen binding scaffold, a ligand or a receptor. The chain of a single chain SAR may bind to one antigen or more than one antigen (e.g., two, three, four etc.). The chain of a single chain NKp30 SAR may further comprise one or more adaptors (e.g., RZIP, EZIP, NKG2D-YA, NKG2D-FA etc.).

In some embodiments, the NKp30 SAR of the disclosure comprises a molecule of the general formula: AABD(n)-optional NKp30 Ig domain, NKp30 hinge domain-NKp30 transmembrane domain-optional-intracellular costimulatory domain(n)-NKp30 intracellular signaling domain wherein n is 1 or more. In one embodiment, n is at least 2, for example 2, 3, 4 or 5. The AABD (autonomous antigen binding domain) forms the antigen binding domain and is located at the extracellular side when expressed in a cell.

In an embodiment, the AABD is a fully human vH domain or a humanized vH domain. In an embodiment, the AABD is a fully human single VH (SVH) domain or a humanized SVH domain. An SVH domain, also known as an autonomous vH domain, can bind to a target in the absence of a vL domain.

In an embodiment, the AABD is a fully human vHH domain or a humanized vHH domain.

In an embodiment, the AABD is a non-immunoglobulin antigen binding scaffold such as a DARPIN, an affibody, a ZIP domain (e.g., RZIP, EZIP, E4, R4 etc.), an affilin, an adnectin, an affitin, an obodies, a repebody, a fynomer, an alphabody, an avimer, an atrimer, a centyrin, a pronectin, an anticalin, a kunitz domain, an Armadillo repeat protein or a fragment thereof; a receptor (e.g., NKp30, CD16-F158V, NKG2D), a ligand (e.g., APRIL, Thrombopoietin) and the like.

In some embodiments, the NKp30 SAR of the disclosure comprises a molecule of the general formula:

scFv(n)-NKp30 Ig domain-NKp30 hinge domain-NKp30 transmembrane domain-optional-intracellular costimulatory domain(n)-optional NKp30 intracellular signaling domain, wherein n is 1 or more. In certain embodiments, the disclosure provides a novel platform of synthetic antigen receptors, designated NKp30-SARs, containing the entire or partial sequence of two NKp30 chains.

The disclosure provides that the vL fragment of an antibody can be joined to one of the two NKp30 chains and the vH fragment can be joined to the other NKp30 chain. When the two such chains (e.g., vL-NKp30 and vH-NKp30) are co-expressed in the same cell, the vL and vH fragments can bind their cognate antigen and transmit a T, NK cell or macrophage signal. In particular, T cells expressing such NKp30-SAR when exposed to a cell line expressing the cognate target antigen can activate NFAT signaling, induce IL2 production, promote T cell proliferation, promote T cell activation and exert cytotoxicity. In another embodiment, NK cells expressing such NKp30-SAR when exposed to a cell line expressing the cognate target antigen can promote NK cell proliferation, promote NK cell activation and exert cytotoxicity. The expression and activity of the NKp30-SAR can be further increased by incorporation of a linker between the vL/vH and the NKP30 fragments. In particular, the IgCL (SEQ ID NO (DNA): 1142 and SEQ ID NO (PRT): 3536) and IgCH domains (SEQ ID NO (DNA): 1143-1157 and SEQ ID NO (PRT): 3537-3551) derived from antibodies serve as useful linkers between the vL/vH and NKp30 fragments. Additional Ig like domains are known in the art (e.g., Table 13; SEQ ID NO (DNA):1168-1175 and SEQ ID NO (PRT):3562-3569) and can serve as useful linkers in alternate embodiment of the disclosure.

In another embodiment, a costimulatory domain is also incorporated in the NKp30 chain(s) of NKp30-SAR. Exemplary costimulatory domains include costimulatory domains of 41BB, CD28, OX40 and 2B4 etc. (Table 30; SEQ ID NO (DNA): 1565-1572 and SEQ ID NO (PRT): 3959-3966). Collectively, the above results provide a novel platform for adoptive cellular therapy that overcomes some of the design limitations of SAR and also provide a complementary approach to SARs.

The two chains of NKp30-SARs described herein may be encoded by a single polynucleotide chain and translated into a single polypeptide chain, which is subsequently cleaved into different proteins. The two chains of NKp30-SARs described herein may be expressed using two distinct promoters and encoded by two separate polynucleotide chains. The two chains of NKp30-SARs described herein may be encoded by a single vector. The two chains of NKp30-SARs described herein may be encoded by a two different vector. The nucleic acid molecule encoding a NKp30-SAR can comprise one or more leader sequences (also known as a signal peptide). In one embodiment, each functional unit (e.g., an antigen binding domain joined to a CD3z chain plus Furine-SGSG-cleavable linker) of a NKp30-SAR can be preceded by a leader sequence which directs the NKp30-SAR to the cell surface as a type I transmembrane protein. In one embodiment, the antigen-binding domain of NKp30-SAR is extracellular-facing. In some embodiments, the leader sequence comprises the nucleic acid sequence of any of SEQ ID NO: 31 to 34 and amino acid sequences of SEQ ID NO: 2425 to 2428. In some embodiments, short nucleic acid sequences (3-9 nucleic acids) comprising restriction enzyme sites are located between the different subunits of a NKp30-SAR, e.g., between a signal sequence and the antigen binding domain of the NKp30-SAR or between the antigen binding and the NKp30 chain.

In certain embodiments, the disclosure provides a novel platform of synthetic antigen receptors, designated NKp30-SARs, containing two chains, one of which incorporates the partial or entire region of NKp30.

In alternate embodiment, the disclosure provides a double chain NKp30 SARs where one of the chains comprises a partial or entire region of NKp30 extracellular domain. In an embodiment, the disclosure provides double chain NKp30 SARs where one of the chains comprises a partial or entire region of NKp30 hinge domain. In an embodiment, the disclosure provides a double chain NKp30 SAR where one of the chains comprises partial or entire region of NKp30 transmembrane domain. In an embodiment, the disclosure provides a double chain NKp30 SAR where one of the chains comprises a partial or entire region of NKp30 cytosolic domain.

The disclosure provides that the vL fragment of an antibody can be joined to a NKp30 chain and the vH fragment can be joined to the another signaling chain, such as CD3z, FcRγ. CD16, NKp44, NKp46, TCRα constant chain, TCRβ constant chain, TCRy constant chain or TCRδ constant chain etc. Alternatively, the disclosure provides that the vH fragment of an antibody can be joined to a NKp30 chain and the vL fragment can be joined to the another signaling chain, such as CD3z, FcRγ, CD16, NKp44, NKp46, TCRα constant chain, TCRβ constant chain, TCRγ constant chain or TCRδ constant chain etc. When the two such chains (e.g., vL-NKp30 and vH-CD3z) are co-expressed in the same cell, the vL and vH fragments can bind their cognate antigen and transmit a T cell signal. In particular, T cells expressing such NKp30-hererodimeric SAR when exposed to a cell line expressing the cognate target antigen can activate NFAT signaling, induce IL2 production, promote T cell proliferation, promote T cell activation and exert cytotoxicity. In another exemplary embodiment, NK cells expressing such NKp30-SAR when exposed to a cell line expressing the cognate target antigen can induce IL2 production, promote NK cell proliferation, promote NK cell activation or exert cytotoxicity. The expression and activity of the NKp30-heterodimeic SAR can be further increased by incorporation of a linker between the vL/vH and the NKp30 and the other signaling chains (e.g., CD3z, FcRγ, NKp30, NKp44, NKp46 etc.). In particular, the IgCL (SEQ ID NO (DNA): 1142 and SEQ ID NO (PRT): 3536) and IgCH domains (SEQ ID NO (DNA): 1143-1157 and SEQ ID NO (PRT): 3537-3551) derived from antibodies serve as useful linkers between the vL/vH and NKp30 fragments.

In certain embodiments, the disclosure provides a novel platform of synthetic antigen receptors, designated NKp44-SARs, containing the entire or partial sequence of a NKp44 chain. The nucleic acid sequences of the NKp44 chains that can be used in the construction of NKp44 SARs are provided in SEQ ID NO: 1381 to 1394. The corresponding amino acid sequences are provided in SEQ ID NO: 3775 to 3788, respectively.

In an embodiment, the disclosure provides a single chain NKp44 SAR comprising a partial or entire region of NKp44. In alternate embodiment, the disclosure provides a single chain NKp44 SARs comprising a partial or entire region of NKp44 extracellular domain. In an embodiment, the disclosure provides NKp44 SARs comprising a partial or entire region of NKp44 hinge domain. In an embodiment, the disclosure provides a NKp44 SAR comprising partial or entire region of NKp44 transmembrane domain. In an embodiment, the disclosure provides a NKp44 SAR comprising a partial or entire region of NKp44 cytosolic domain. In an embodiment, a NKp44 SAR comprises the NKp44 Ig domain that is attached via NKp44 hinge domain to NKp44 transmembrane domain and NKp44 cytosolic domain. In an embodiment, a NKp44 SAR comprises the NKp44 hinge domain that is attached to NKp44 transmembrane domain and NKp44 cytosolic domain.

It is to be understood that the different NKp44 domains (i.e., extracellular, Ig domain, hinge, transmembrane and cytosolic) that may be used in the construction of the SAR may comprise their entire sequence or a deletion mutant or a variant as long as the domain retains at least some of its functional property.

In an embodiment, the antigen binding domain of the NKp44 SAR comprises a scFv, a vL, vH, Fv, vHH, FHVH, a single domain antibody, a non-immunoglobulin antigen binding scaffold, a ligand or a receptor. The chain of a single chain SAR may bind to one antigen or more than one antigen (e.g., two, three, four etc.). The chain of a single chain NKp44 SAR may further comprise one or more adaptors (e.g., RZIP, EZIP, NKG2D-YA, NKG2D-FA etc.).

In some embodiments, the NKp44 SAR of the disclosure comprises a molecule of the general formula: AABD(n)-optional NKp44 Ig domain, NKp44 hinge domain-NKp44 transmembrane domain-optional-intracellular costimulatory domain(n)-NKp44 intracellular signaling domain wherein n is 1 or more. In one embodiment, n is at least 2, for example 2, 3, 4 or 5. The AABD (autonomous antigen binding domain) forms the antigen binding domain and is located at the extracellular side when expressed in a cell.

In an embodiment, the AABD is a fully human vH domain or a humanized vH domain. In an embodiment, the AABD is a fully human single VH (SVH) domain or a humanized SVH domain. An SVH domain, also known as an autonomous vH domain, can bind to a target in the absence of a vL domain.

In an embodiment, the AABD is a fully human vHH domain or a humanized vHH domain.

In an embodiment, the AABD is a non-immunoglobulin antigen binding scaffold such as a DARPIN, an affibody, a ZIP domain (e.g., RZIP, EZIP, E4, R4 etc.), an affilin, an adnectin, an affitin, an obodies, a repebody, a fynomer, an alphabody, an avimer, an atrimer, a centyrin, a pronectin, an anticalin, a kunitz domain, an Armadillo repeat protein or a fragment thereof; a receptor (e.g., NKp44, NKG2D), a ligand (e.g., APRIL, Thrombopoietin) and the like.

In some embodiments, the NKp44 SAR of the disclosure comprises a molecule of the general formula:

scFv(n)-NKp44 Ig domain-NKp44 hinge domain-NKp44 transmembrane domain-optional-intracellular costimulatory domain(n)-optional NKp44 intracellular signaling domain, wherein n is 1 or more.

In certain embodiments, the disclosure provides a novel platform of synthetic antigen receptors, designated NKp44-SARs, containing the entire or partial sequence of two NKp44 chains. The disclosure provides that the vL fragment of an antibody can be joined to one of the two NKp44 chains and the vH fragment can be joined to the other NKp44 chain. When the two such chains (e.g., vL-NKp44 and vH-NKp44) are co-expressed in the same cell, the vL and vH fragments can bind their cognate antigen and transmit a T or NK cell signal. In particular, T cells expressing such NKp44-SAR when exposed to a cell line expressing the cognate target antigen can activate NFAT signaling, induce IL2 production, promote T cell proliferation, promote T cell activation and exert cytotoxicity. In another embodiment, NK cells expressing such NKp44-SAR when exposed to a cell line expressing the cognate target antigen can promote NK cell proliferation, promote NK cell activation and exert cytotoxicity. The expression and activity of the NKp44-SAR can be further increased by incorporation of a linker between the vL/vH and the NKP30 fragments. In particular, the IgCL (SEQ ID NO (DNA): 1142 and SEQ ID NO (PRT): 3536) and IgCH domains (SEQ ID NO (DNA): 1143-1157 and SEQ ID NO (PRT): 3537-3551) derived from antibodies serve as useful linkers between the vL/vH and NKp44 fragments. Additional Ig like domains are known in the art (e.g., Table 13; SEQ ID NO (DNA):1168-1175 and SEQ ID NO (PRT):3562-3569) and can serve as useful linkers in alternate embodiment of the disclosure.

In another embodiment, a costimulatory domain is also incorporated in the NKp44 chain(s) of NKp44-SAR. Exemplary costimulatory domains include costimulatory domains of 41BB, CD28, OX40 and 2B4 etc. (Table 30; SEQ ID NO (DNA): 1565-1572 and SEQ ID NO (PRT): 3959-3966). Collectively, the above results provide a novel platform for adoptive cellular therapy that overcomes some of the design limitations of SAR and also provide a complementary approach to SARs.

The two chains of NKp44-SARs described herein may be encoded by a single polynucleotide chain and translated into a single polypeptide chain, which is subsequently cleaved into different proteins. The two chains of NKp44-SARs described herein may be expressed using two distinct promoters and encoded by two separate polynucleotide chains. The two chains of NKp44-SARs described herein may be encoded by a single vector. The two chains of NKp44-SARs described herein may be encoded by a two different vector. The nucleic acid molecule encoding a NKp44-SAR can comprise one or more leader sequences (also known as a signal peptide). In one embodiment, each functional unit (e.g., an antigen binding domain joined to a CD3z chain plus Furine-SGSG-cleavable linker) of a NKp44-SAR can be preceded by a leader sequence which directs the NKp44-SAR to the cell surface as a type I transmembrane protein. In one embodiment, the antigen-binding domain of NKp44-SAR is extracellular-facing. In some embodiments, the leader sequence comprises the nucleic acid sequence of any of SEQ ID NO: 31 to 34 and amino acid sequences of SEQ ID NO: 2425 to 2428. In some embodiments, short nucleic acid sequences (3-9 nucleic acids) comprising restriction enzyme sites are located between the different subunits of a NKp44-SAR, e.g., between a signal sequence and the antigen binding domain of the NKp44-SAR or between the antigen binding and the NKp44 chain.

In certain embodiments, the disclosure provides a novel platform of synthetic antigen receptors, designated NKp44-SARs, containing two chains, one of which incorporates the partial or entire region of NKp44.

In alternate embodiment, the disclosure provides a double chain NKp44 SARs where one of the chains comprises a partial or entire region of NKp44 extracellular domain. In an embodiment, the disclosure provides double chain NKp44 SARs where one of the chains comprises a partial or entire region of NKp44 hinge domain. In an embodiment, the disclosure provides a double chain NKp44 SAR where one of the chains comprises partial or entire region of NKp44 transmembrane domain. In an embodiment, the disclosure provides a double chain NKp44 SAR where one of the chains comprises a partial or entire region of NKp44 cytosolic domain.

The disclosure provides that the vL fragment of an antibody can be joined to a NKp44 chain and the vH fragment can be joined to the another signaling chain, such as CD3z, FcRγ. CD16, NKp30, NKp46, TCRα constant chain, TCRβ constant chain, TCRy constant chain or TCRδ constant chain etc. Alternatively, the disclosure provides that the vH fragment of an antibody can be joined to a NKp44 chain and the vL fragment can be joined to the another signaling chain, such as CD3z, FcRγ, CD16, NKp30, NKp46, TCRα constant chain, TCRβ constant chain, TCRγ constant chain or TCRδ constant chain etc. When the two such chains (e.g., vL-NKp44 and vH-CD3z) are co-expressed in the same cell, the vL and vH fragments can bind their cognate antigen and transmit a T cell signal. In particular, T cells expressing such NKp44-hererodimeric SAR when exposed to a cell line expressing the cognate target antigen can activate NFAT signaling, induce IL2 production, promote T cell proliferation, promote T cell activation and exert cytotoxicity. In another exemplary embodiment, NK cells expressing such NKp44-SAR when exposed to a cell line expressing the cognate target antigen can induce IL2 production, promote NK cell proliferation, promote NK cell activation or exert cytotoxicity. The expression and activity of the NKp44-heterodimeic SAR can be further increased by incorporation of a linker between the vL/vH and the NKp44 and the other signaling chains (e.g., CD3z, FcRγ, NKp44, NKp44, NKp46 etc.). In particular, the IgCL (SEQ ID NO (DNA): 1142 and SEQ ID NO (PRT): 3536) and IgCH domains (SEQ ID NO (DNA): 1143-1157 and SEQ ID NO (PRT): 3537-3551) derived from antibodies serve as useful linkers between the vL/vH and NKp44 fragments. Additional Ig like domains are known in the art (e.g., Table 13; SEQ ID NO (DNA):1168-1175 and SEQ ID NO (PRT):3562-3569) and can serve as useful linkers in alternate embodiment of the disclosure.

In certain embodiments, the disclosure provides a novel platform of synthetic antigen receptors, designated NKp46-SARs, containing the entire or partial sequence of a NKp46 chain.

The nucleic acid sequences of the NKp46 chains that can be used in the construction of NKp46 SARs are provided in SEQ ID NO: 1381 to 1394 (Table 25). The corresponding amino acid sequences are provided in SEQ ID NO: 3775 to 3788, respectively (Table 25).

In an embodiment, the disclosure provides a single chain NKp46 SAR comprising a partial or entire region of NKp46. In alternate embodiment, the disclosure provides a single chain NKp46 SARs comprising a partial or entire region of NKp46 extracellular domain. In an embodiment, the disclosure provides NKp46 SARs comprising a partial or entire region of NKp46 hinge domain. In an embodiment, the disclosure provides a NKp46 SAR comprising partial or entire region of NKp46 transmembrane domain. In an embodiment, the disclosure provides a NKp46 SAR comprising a partial or entire region of NKp46 cytosolic domain.

In an embodiment, a NKp46 SAR comprises the NKp46 Ig domain that is attached via NKp46 hinge domain to NKp46 transmembrane domain and NKp46 cytosolic domain. In an embodiment, a NKp46 SAR comprises the NKp46 hinge domain that is attached to NKp46 transmembrane domain and NKp46 cytosolic domain.

It is to be understood that the different NKp46 domains (i.e., extracellular, Ig domain, hinge, transmembrane and cytosolic) that may be used in the construction of the SAR may comprise their entire sequence or a deletion mutant or a variant as long as the domain retains at least some of its functional property.

In an embodiment, the antigen binding domain of the NKp46 SAR comprises a scFv, a vL, vH, Fv, vHH, FHVH, a single domain antibody, a non-immunoglobulin antigen binding scaffold, a ligand or a receptor. The chain of a single chain SAR may bind to one antigen or more than one antigen (e.g., two, three, four etc.). The chain of a single chain NKp46 SAR may further comprise one or more adaptors (e.g., RZIP, EZIP, NKG2D-YA, NKG2D-FA etc.).

In some embodiments, the NKp46 SAR of the disclosure comprises a molecule of the general formula: AABD(n)-optional NKp46 Ig domain, NKp46 hinge domain-NKp46 transmembrane domain-optional-intracellular costimulatory domain(n)-NKp46 intracellular signaling domain wherein n is 1 or more. In one embodiment, n is at least 2, for example 2, 3, 4 or 5. The AABD (autonomous antigen binding domain) forms the antigen binding domain and is located at the extracellular side when expressed in a cell.

In an embodiment, the AABD is a fully human vH domain or a humanized vH domain. In an embodiment, the AABD is a fully human single VH (SVH) domain or a humanized SVH domain. An SVH domain, also known as an autonomous vH domain, can bind to a target in the absence of a vL domain.

In an embodiment, the AABD is a fully human vHH domain or a humanized vHH domain.

In an embodiment, the AABD is a non-immunoglobulin antigen binding scaffold such as a DARPIN, an affibody, a ZIP domain (e.g., RZIP, EZIP, E4, R4 etc.), an affilin, an adnectin, an affitin, an obodies, a repebody, a fynomer, an alphabody, an avimer, an atrimer, a centyrin, a pronectin, an anticalin, a kunitz domain, an Armadillo repeat protein or a fragment thereof; a receptor (e.g., NKp46, NKG2D), a ligand (e.g., APRIL, Thrombopoietin) and the like.

In some embodiments, the NKp46 SAR of the disclosure comprises a molecule of the general formula:

scFv(n)-NKp46 Ig domain-NKp46 hinge domain-NKp46 transmembrane domain-optional-intracellular costimulatory domain(n)-optional NKp46 intracellular signaling domain, wherein n is 1 or more.

In certain embodiments, the disclosure provides a novel platform of synthetic antigen receptors, designated NKp46-SARs, containing the entire or partial sequence of two NKp46 chains. The disclosure provides that the vL fragment of an antibody can be joined to one of the two NKp46 chains and the vH fragment can be joined to the other NKp46 chain. When the two such chains (e.g., vL-NKp46 and vH-NKp46) are co-expressed in the same cell, the vL and vH fragments can bind their cognate antigen and transmit a T or NK cell signal. In particular, T cells expressing such NKp46-SAR when exposed to a cell line expressing the cognate target antigen can activate NFAT signaling, induce IL2 production, promote T cell proliferation, promote T cell activation and exert cytotoxicity. In another embodiment, NK cells expressing such NKp46-SAR when exposed to a cell line expressing the cognate target antigen can promote NK cell proliferation, promote NK cell activation and exert cytotoxicity. The expression and activity of the NKp46-SAR can be further increased by incorporation of a linker between the vL/vH and the NKP30 fragments. In particular, the IgCL (SEQ ID NO (DNA): 1142 and SEQ ID NO (PRT): 3536) and IgCH domains (SEQ ID NO (DNA): 1143-1157 and SEQ ID NO (PRT): 3537-3551) derived from antibodies serve as useful linkers between the vL/vH and NKp46 fragments. Additional Ig like domains are known in the art (e.g., Table 13; SEQ ID NO (DNA):1168-1175 and SEQ ID NO (PRT):3562-3569) and can serve as useful linkers in alternate embodiment of the disclosure.

In another embodiment, a costimulatory domain is also incorporated in the NKp46 chain(s) of NKp46-SAR. Exemplary costimulatory domains include costimulatory domains of 41BB, CD28, OX40 and 2B4 etc. (Table 30; SEQ ID NO (DNA): 1565-1572 and SEQ ID NO (PRT): 3959-3966). Collectively, the above results provide a novel platform for adoptive cellular therapy that overcomes some of the design limitations of SAR and also provide a complementary approach to SARs.

The two chains of NKp46-SARs described herein may be encoded by a single polynucleotide chain and translated into a single polypeptide chain, which is subsequently cleaved into different proteins. The two chains of NKp46-SARs described herein may be expressed using two distinct promoters and encoded by two separate polynucleotide chains. The two chains of NKp46-SARs described herein may be encoded by a single vector. The two chains of NKp46-SARs described herein may be encoded by a two different vector. The nucleic acid molecule encoding a NKp46-SAR can comprise one or more leader sequences (also known as a signal peptide). In one embodiment, each functional unit (e.g., an antigen binding domain joined to a CD3z chain plus Furine-SGSG-cleavable linker) of a NKp46-SAR can be preceded by a leader sequence which directs the NKp46-SAR to the cell surface as a type I transmembrane protein. In one embodiment, the antigen-binding domain of NKp46-SAR is extracellular-facing. In some embodiments, the leader sequence comprises the nucleic acid sequence of any of SEQ ID NO: 31 to 34 and amino acid sequences of SEQ ID NO: 2425 to 2428. In some embodiments, short nucleic acid sequences (3-9 nucleic acids) comprising restriction enzyme sites are located between the different subunits of a NKp46-SAR, e.g., between a signal sequence and the antigen binding domain of the NKp46-SAR or between the antigen binding and the NKp46 chain.

In certain embodiments, the disclosure provides a novel platform of synthetic antigen receptors, designated NKp46-SARs, containing two chains, one of which incorporates the partial or entire region of NKp46.

In alternate embodiment, the disclosure provides a double chain NKp46 SARs where one of the chains comprises a partial or entire region of NKp46 extracellular domain. In an embodiment, the disclosure provides double chain NKp46 SARs where one of the chains comprises a partial or entire region of NKp46 hinge domain. In an embodiment, the disclosure provides a double chain NKp46 SAR where one of the chains comprises partial or entire region of NKp46 transmembrane domain. In an embodiment, the disclosure provides a double chain NKp46 SAR where one of the chains comprises a partial or entire region of NKp46 cytosolic domain.

The disclosure provides that the vL fragment of an antibody can be joined to a NKp46 chain and the vH fragment can be joined to the another signaling chain, such as CD3z, FcRγ. CD16, NKp30, NKp44, TCRα constant chain, TCRβ constant chain, TCRy constant chain or TCRδ constant chain etc. Alternatively, the disclosure provides that the vH fragment of an antibody can be joined to a NKp46 chain and the vL fragment can be joined to the another signaling chain, such as CD3z, FcRγ, CD16, NKp30, NKp44, TCRα constant chain, TCRβ constant chain, TCRγ constant chain or TCRδ constant chain etc. When the two such chains (e.g., vL-NKp46 and vH-CD3z) are co-expressed in the same cell, the vL and vH fragments can bind their cognate antigen and transmit a T cell signal. In particular, T cells expressing such NKp46-hererodimeric SAR when exposed to a cell line expressing the cognate target antigen can activate NFAT signaling, induce IL2 production, promote T cell proliferation, promote T cell activation and exert cytotoxicity. In another exemplary embodiment, NK cells expressing such NKp46-SAR when exposed to a cell line expressing the cognate target antigen can induce IL2 production, promote NK cell proliferation, promote NK cell activation or exert cytotoxicity. The expression and activity of the NKp46-heterodimeic SAR can be further increased by incorporation of a linker between the vL/vH and the NKp46 and the other signaling chains (e.g., CD3z, FcRγ, NKp46, NKp46, NKp46 etc.). In particular, the IgCL (SEQ ID NO (DNA): 1142 and SEQ ID NO (PRT): 3536) and IgCH domains (SEQ ID NO (DNA): 1143-1157 and SEQ ID NO (PRT): 3537-3551) derived from antibodies serve as useful linkers between the vL/vH and NKp46 fragments.

The disclosure also provides SARs based on the extracellular, transmembrane and cytosolic domains of other NK receptor including co-stimulatory receptors (SEQ ID NO: 9860-9993). As SARs are modular in format, the hu-mROO5-scFv targeting CD19 in these constructs can be switched with other antigen binding domains described in Tables 3-7 to generate novel unispecific and bispecific SARs.

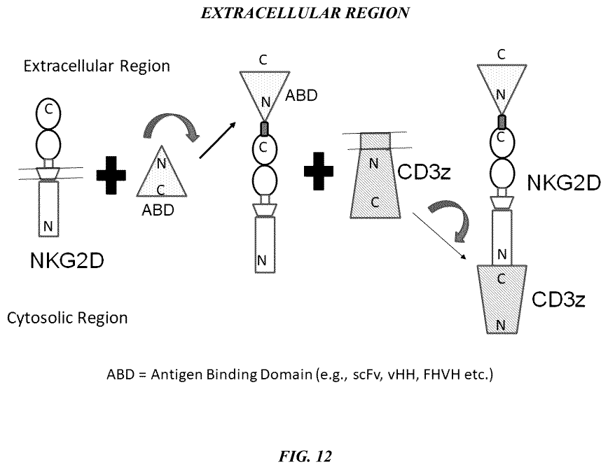

NKG2D is a type II protein, in which the N-terminus is located intracellularly. Although CAR based on fragments of NKG2D have been described in the art, they lack the native configuration of NKG2D cytosolic and transmembrane domains. The disclosure provides a SAR in which the N-terminus of a polypetide comprising one or more antigen binding domains (e.g., AABD, scFv) is fused in frame to a polypeptide comprising from N-terminus to C-terminus the intracellular, transmembrane, and extracellular domain of NKG2D or a Type II membrane protein via an optional linker. The schematic of such a construct is provided in FIG. 12. Exemplary such SARs are provided in SEQ ID NO: 7686-7687. Further, the N-terminus of the cytosolic domain of an adaptor (e.g., CD3z) with an ATG start codon can be fused to the N-terminus of NKG2D to provide an activation domain to the SAR. The disclosure also provides SARs in which the N-terminaus domain of an antigen binding domain is fused to the extracellular domain of NKG2C, NKG2A, NKG2E and NKG2F receptors. This scheme can be used to generate a fusion protein between any Type I protein, including a Type I protein comprising an antigen binding domain, and a Type II protein. The scheme can be also used to generate fusion comprising only the hinge, transmembrane and cytosolic domains of the Type II receptor and lacking its extracellular domain.

Finally, the disclosure also provides a method to generate heterodimeric SAR based on Type II proteins in which one antigen binding domain is attached to the C-terminus of one receptor chain and a second antigen binding domain is attached to the C-terminus of a second heterdimeric chain. An exemplary such receptors comprising NKG2E and CD94 is provided in SEQ ID NO: 10341.

In certain embodiments, the disclosure provides that novel platform of synthetic antigen receptors, comprising the partial or entire sequence derived from two CD3z chains can be functionally expressed in immune cells, such as NK cells, NK92 cell line, monocytes/macrophages and neutrophils, which lack the endogenous TCR chains. In certain embodiments, the disclosure provides that novel platform of SAR, comprising the partial or entire sequence derived from two CD3z chains that can be expressed in iPSC cells, embryonic stem cells or hematopoietic stem cells, which can be differentiated to generate immune cells, such as NK cells, monocytes/macrophages and neutrophils, expressing the zSAR. The nucleic acid sequences of the exemplary CD3z chains that can be used in the construction of zSAR are provided in SEQ ID NO: 1090 and 1096. The corresponding amino acid sequences are provided in SEQ ID NO: 3484 and 3490, respectively. The disclosure provides that the vL fragment of an antibody can be joined to one of the two CD3z chains and the vH fragment can be joined to the other CD3z chain. When the two such chains (e.g, vL-CD3z and vH-CD3z) are co-expressed in the same cell, the vL and vH fragments can bind their cognate antigen and transmit a T cell signal. In particular, NK cells expressing such zSAR when exposed to a cell line expressing the cognate target antigen can show increased proliferation, activation and exert cytotoxicity. The expression and activity of the zSAR can be further increased by incorporation of a linker between the vL/vH and the CD3z fragments. In particular, the IgCL (SEQ ID NO (DNA): 1142 and SEQ ID NO (PRT): 3536) and IgCH domains (SEQ ID NO (DNA): 1143-1157 and SEQ ID NO (PRT): 3537-3551) derived from antibodies serve as useful linkers between the vL/vH and CD3z fragments. Additional Ig like domains are known in the art (e.g., Table 13; SEQ ID NO (DNA):1168-1175 and SEQ ID NO (PRT):3562-3569) and can serve as useful linkers in alternate embodiment of the disclosure.

In another embodiment, a costimulatory domain is also incorporated in the CD3z chain(s) of zSAR. Exemplary costimulatory domains include costimulatory domains of 41BB and CD28. CD3z chains containing 41BB and CD28 costimulatory domains are presented in SEQ ID NO: 1100, 1102 and 1099 and 1101, respectively. Other exemplary costimulatory domains (e.g., OX40 and 2B4) that can be substituted for the 41BB and CD28 costimulatory domains are provided in Table 30 of provisional application. Collectively, the above results provide a novel platform for adoptive cellular therapy that overcomes some of the design limitations of CARs and also provide a complementary approach to SIRs.

The two chains of zSARs described herein may be encoded by a single polynucleotide chain and translated into a single polypeptide chain, which is subsequently cleaved into different proteins. The two chains of zSARs described herein may be expressed using two distinct promoters and encoded by two separate polynucleotide chains. The two chains of zSARs described herein may be encoded by a single vector. The two chains of zSARs described herein may be encoded by a two different vector. The nucleic acid molecule encoding a zSAR can comprise one or more leader sequences (also known as a signal peptide). In one embodiment, each functional unit (e.g., an antigen binding domain joined to a CD3z chain plus Furine-SGSG-cleavable linker) of a zSAR can be preceded by a leader sequence which directs the zSAR to the cell surface as a type I transmembrane protein. In one embodiment, the antigen-binding domain of zSAR is extracellular-facing. In some embodiments, the leader sequence comprises the nucleic acid sequence of any of SEQ ID NO: 31 to 34 and amino acid sequences of SEQ ID NO: 2425 to 2428. In some embodiments, short nucleic acid sequences (3-9 nucleic acids) comprising restriction enzyme sites are located between the different subunits of a zSAR, e.g., between a signal sequence and the antigen binding domain of the zSAR or between the antigen binding and the CD3z chain.

An exemplary zSAR targeting CD19 that can be expressed in immune cells (e.g., NK cells, monocytes/macrophages, neutrophils, NK92 cell line etc.) or stem cells (e.g., iPSC, hematopoietic stem cells etc.) that can give rise to immune cells is CD8SP-hu-mROO5-1-vL-IgCL-Bam-CD3zECDTMCP-opt-F-P2A-Spe-SP-Bst-hu-mROO5-1-vH-IgG1-CH1-KPN-CD3zECDTMCP (SEQ ID NO: 2306). Additional exemplary zSAR targeting CD19 that can be functionally expressed in NK cells are presented in SEQ ID NO (DNA): 2287-2291.

The disclosure also provides a zSAR in which the Va, b, g, d domains of a TCR is used as the antigen binding domain. Such a SAR acts like a uTCR-SAR.

The one or both CD3 domains in zSAR can be replaced by other signaling adaptors, such as DAP10, DAP12 or FcRγ or fragments or variants thereof to generate novel SARs comprising these adaptors.

The disclosure provides, single chain, double chain and double chain hetero-dimeric SARs comprising the partial or entire region of DAP10 (SEQ ID NO(DNA): 1349-1350). Exemplary single chain, double chain and double chain hetero-dimeric DAP10 SARs of the disclosure are provided in Tables 32 and 33 of provisional application.

In an embodiment, the disclosure provides a single chain DAP10 SAR comprising a partial or entire region of DAP10. In alternate embodiment, the disclosure provides a single chain DAP10 SARs comprising a partial or entire region of CD16 extracellular domain. In an embodiment, the disclosure provides a DAP10 SAR comprising partial or entire region of DAP10 transmembrane domain. In an embodiment, the disclosure provides a DAP10 SAR comprising a partial or entire region of DAP10 cytosolic domain. An exemplary SARS comprising DAP10 is CD8SP-Sph-BCMA-FHVH93-Kpn-G4S-EcoRI-Xho-DAP10-opt1-F-P2A-SpeXba-PAC and is represented by SEQ ID NO (DNA): 2002 and SEQ ID NO (PRT): 4396.

In an embodiment, the disclosure provides a single chain DAP10 SAR comprising a partial or entire region of DAP10 that is fused in frame at its C-terminus to sequence encoding an activation domain. In an embodiment, the activation domain is derived from the cytosolic domain of CD3z (SEQ ID NO (DNA): 1562-1564 and SEQ ID NO (PRT): 3956-3958). An exemplary SARS comprising DAP10 fused to CD3z activation domain is CD8SP-Sph-BCMA-FHVH93-Kpn-G4S-EcoR1-Xho-DAP10-opt1-Spe-CD3zCP-opt1-F-P2A-SpeXba-PAC (SEQ ID NO (DNA): 2037 and SEQ ID NO (PRT): 4431).

It is to be understood that the different DAP10 domains that may be used in the construction of the SAR may comprise their entire sequence or a deletion mutant or a variant as long as the domain retains at least some of its functional property.

In an embodiment, the antigen binding domain of the DAP10 SAR comprises a scFv, a vL, vH, Fv, vHH, FHVH, a single domain antibody, a non-immunoglobulin antigen binding scaffold, a ligand or a receptor. The chain of a single chain SAR may bind to one antigen or more than one antigen (e.g., two, three, four etc.). The chain of a single chain CD16 SAR may further comprise one or more adaptors (e.g., RZIP, EZIP, NKG2D-YA, NKG2D-FA etc.).

In some embodiments, the DAP10 SAR of the disclosure comprises a molecule of the general formula: AABD(n)-DAP10 hinge domain-DAP10 transmembrane domain-DAP10-intracellular signaling domain-optional activation domain wherein n is 1 or more. In one embodiment, n is at least 2, for example 2, 3, 4 or 5. The AABD (autonomous antigen binding domain) forms the antigen binding domain and is located at the extracellular side when expressed in a cell.

In an embodiment, the AABD is a fully human vH domain or a humanized vH domain. In an embodiment, the AABD is a fully human single VH (SVH) domain or a humanized SVH domain. An SVH domain, also known as an autonomous vH domain, can bind to a target in the absence of a vL domain.

In an embodiment, the AABD is a fully human vHH domain or a humanized vHH domain.

In an embodiment, the AABD is a non-immunoglobulin antigen binding scaffold such as a DARPIN, an affibody, a ZIP domain (e.g., RZIP, EZIP, E4, R4 etc.), an affilin, an adnectin, an affitin, an obodies, a repebody, a fynomer, an alphabody, an avimer, an atrimer, a centyrin, a pronectin, an anticalin, a kunitz domain, an Armadillo repeat protein or a fragment thereof, a receptor (e.g., CD16-F158V, NKG2D), a ligand (e.g., APRIL, Thrombopoietin) and the like.

In certain embodiments, the disclosure provides a novel platform of synthetic antigen receptors, designated DAP10-SARs, containing two DAP10 chains. The disclosure provides that the vL fragment of an antibody can be joined to one of the two DAP10 chains and the vH fragment can be joined to the other DAP10 chain. When the two such chains (e.g., vL-DAP10 and vH-DAP10) are co-expressed in the same cell, the vL and vH fragments can bind their cognate antigen and transmit a T cell signal. In particular, T cells expressing such DAP10-SAR when exposed to a cell line expressing the cognate target antigen can activate NFAT signaling, induce IL2 production, promote T cell proliferation, promote T cell activation and exert cytotoxicity. In another embodiment, NK cells expressing such DAP10-SAR when exposed to a cell line expressing the cognate target antigen can promote NK cell proliferation, promote NK cell activation and exert cytotoxicity. In particular, the IgCL (SEQ ID NO (DNA): 1142 and SEQ ID NO (PRT): 3536) and IgCH domains (SEQ ID NO (DNA): 1143-1157 and SEQ ID NO (PRT): 3537-3551) derived from antibodies serve as useful linkers between the vL/vH and DAP10 fragments. Additional Ig like domains are known in the art (e.g., Table 13; SEQ ID NO (DNA):1168-1175 and SEQ ID NO (PRT):3562-3569) and can serve as useful linkers in alternate embodiment of the disclosure.

In another embodiment, a costimulatory domain is also incorporated in the DAP10 chain(s) of DAP10-SAR. Exemplary costimulatory domains include costimulatory domains of 41BB, CD28, OX40 and 2B4 etc. (Table 30; SEQ ID NO (DNA): 1565-1572 and SEQ ID NO (PRT): 3959-3966). Collectively, the above results provide a novel platform for adoptive cellular therapy that overcomes some of the design limitations of SAR and also provide a complementary approach to SARs.

The two chains of DAP10-SARs described herein may be encoded by a single polynucleotide chain and translated into a single polypeptide chain, which is subsequently cleaved into different proteins. The two chains of DAP10-SARs described herein may be expressed using two distinct promoters and encoded by two separate polynucleotide chains. The two chains of DAP10-SARs described herein may be encoded by a single vector. The two chains of DAP10-SARs described herein may be encoded by a two different vector. The nucleic acid molecule encoding a DAP10-SAR can comprise one or more leader sequences (also known as a signal peptide). In one embodiment, each functional unit (e.g., an antigen binding domain joined to a CD3z chain plus Furine-SGSG-cleavable linker) of a DAP10-SAR can be preceded by a leader sequence which directs the DAP10-SAR to the cell surface as a type I transmembrane protein. In one embodiment, the antigen-binding domain of DAP10-SAR is extracellular-facing. In some embodiments, the leader sequence comprises the nucleic acid sequence of any of SEQ ID NO: 31 to 34 and amino acid sequences of SEQ ID NO: 2425 to 2428. In some embodiments, short nucleic acid sequences (3-9 nucleic acids) comprising restriction enzyme sites are located between the different subunits of a DAP10-SAR, e.g., between a signal sequence and the antigen binding domain of the DAP10-SAR or between the antigen binding and the CD3z chain.

The different SARS of this disclosure are modular in design. Therefore, the sequence encoding the DAP10 module (SEQ ID NO: 1349) may be replaced by a sequence encoding different signaling modules (Table 25). Exemplary such modules include DAP12-ECDTMCP-opt1 (SEQ ID NO:1362), DAP12-C35S-ECDTMCP-opt1 (SEQ ID NO: 1366), CD3z-ECD™-opt1 (SEQ ID NO: 1351), mutCD3z-ECD™-opt1 (SEQ ID NO: 1353), CD3z-ECD™-OX40-opt1 (SEQ ID NO: 1357), FcRy-C24S-ECDTMCP-opt1 (SEQ ID NO: 1423), FcRy-ECDTMCP-opt1 (SEQ ID NO: 1419), mutCD3z-ECD™-2B4CP-opt1 (SEQ ID NO: 1426), CD8-hinge-NKG2D-TM-2B4CP-opt1 (SEQ ID NO: 1430), mutCD8-hinge-NKG2D-TM-2B4CP-opt-1 (SEQ ID NO: 1438). The SEQ ID NOs of exemplary SARS in which one or more of the DAP10 modules are replaced with a different signaling modules are presented in Table 33 of provisional application.

In certain embodiments, the disclosure provides a novel platform of synthetic antigen receptors, designated a costimulatory SAR, containing the entire or partial sequence of a co-stimulatory receptor, including but not limited to 4-1BB, CD28, OX40 and 2B4. The nucleic acid sequences of the costimulatory receptor chains that can be used in the construction of co-stimulatory SARs are provided in SEQ ID NO: 1573 to 1580 (Table 25). The corresponding amino acid sequences are provided in SEQ ID NO: 3967 to 3974, respectively. The exemplary single chain, double chain and double chain heterodimeric SAR comprising the entire or partial sequence of exemplary costimulatory receptors are provided in Tables 41 and 42 of provisional application.

In an embodiment, the disclosure provides a single chain 4-1BB SAR comprising a partial or entire region of 4-1BB attached to one or more antigen binding domains. In an embodiment, the disclosure provides a single chain CD28 SAR comprising a partial or entire region of CD28 attached to one or more antigen binding domains. In an embodiment, the disclosure provides a single chain OX40 SAR comprising a partial or entire region of OX40 attached to one or more antigen binding domains. In an embodiment, the disclosure provides a single chain 2B4 SAR comprising a partial or entire region of 2B4 attached to one or more antigen binding domains.

In an embodiment, the disclosure provides a double chain 4-1BB SAR comprising a partial or entire region of 4-1BB attached to one or more antigen binding domains. In an embodiment, the disclosure provides a double chain CD28 SAR comprising a partial or entire region of CD28 attached to one or more antigen binding domains. In an embodiment, the disclosure provides a double chain OX40 SAR comprising a partial or entire region of OX40 attached to one or more antigen binding domains. In an embodiment, the disclosure provides a double chain 2B4 SAR comprising a partial or entire region of 2B4 attached to one or more antigen binding domains.

In an embodiment, the disclosure provides a double chain heterodimeric 4-1BB SAR comprising a partial or entire region of 4-1BB attached to one or more antigen binding domains. In an embodiment, the disclosure provides a double chain heterodimeric CD28 SAR comprising a partial or entire region of CD28 attached to one or more antigen binding domains. In an embodiment, the disclosure provides a double chain heterodimeric OX40 SAR comprising a partial or entire region of OX40 attached to one or more antigen binding domains. In an embodiment, the disclosure provides a double chain heterodimeric 2B4 SAR comprising a partial or entire region of 2B4 attached to one or more antigen binding domains.

It is to be noted that in the case of a double chain heterodimeric SAR, one of the chains may comprise of a co-stimulatory receptor (e.g., 4-1BB, CD28, OX40, 2B4 etc.) while the other chain may comprise of a receptor that is capable of delivering an activation signal (e.g., CD16).

It is to be understood that the different costimulatory receptor domains that may be used in the construction of the SAR may comprise their entire sequence or a deletion mutant or a variant as long as the domain retains at least some of its functional property.

In an embodiment, the antigen binding domain of the co-stimulatory SAR comprises a scFv, a vL, vH, Fv, vHH, FHVH, a single domain antibody, a non-immunoglobulin antigen binding scaffold, a ligand or a receptor. The chain of a single chain SAR may bind to one antigen or more than one antigen (e.g., two, three, four etc.). The chain of a single chain co-stimulatory receptor SAR may further comprise one or more adaptors (e.g., RZIP, EZIP, NKG2D-YA, NKG2D-FA etc.).

In some embodiments, the co-stimulatory SAR of the disclosure comprises a molecule of the general formula: AABD(n)-co-stimulatory receptor hinge domain-co-stimulatory receptor transmembrane domain-co-stimulatory receptor-intracellular signaling domain-optional activation domain wherein n is 1 or more. In one embodiment, n is at least 2, for example 2, 3, 4 or 5. The AABD (autonomous antigen binding domain) forms the antigen binding domain and is located at the extracellular side when expressed in a cell.

In an embodiment, the AABD is a fully human vH domain or a humanized vH domain. In an embodiment, the AABD is a fully human single VH (SVH) domain or a humanized SVH domain. An SVH domain, also known as an autonomous vH domain, can bind to a target in the absence of a vL domain.

In an embodiment, the AABD is a fully human vHH domain or a humanized vHH domain.

The vector encoding SAR generally have a limited capacity to encode a SAR. For example, the size of a SAR polynucleotide affects the titer of the lentiviral or retroviral vector. As such, a SAR that is small in size is desirable. In one aspect, the disclosure describes a unispecific double chain SAR inclusive of two signal peptides and an intervening 2A linker that is less than 1765 nucleotide, less than 1770 nucleotide, less than 1780 nucleotide, less than 1790 nucleotide, less than 1800 nucleotide, less than 1820 nucleotide in size. In one aspect, the disclosure describes a unispecific double chain SAR where one of the chains without the signal sequence is no longer than 815 nucleotide, 820 nucleotide, 825 nucleotides or 850 nucleotides and the second chain without the signal sequence is no longer than 790 nucleotides, 800 nucleotides, 810 nucleotides, 815 nucleotides, 820 nucleotides, 825 nucleotides or 850 nucleotides. In one aspect the SAR has the backbone of a SIR, cTCR, Ab-TCR, AABD-TCR, εTFP, γTFP, δTFP, αPTFP, γδTFP or a TCR. In one aspect the SAR has the backbone of a SIR. In one aspect the SAR has the backbone of a SIR, cTCR, Ab-TCR, AABD-TCR, αPTFP, γδTFP or a TCR with TCRα and TCRβ constant chains. In one aspect the SAR has the backbone of a SIR, cTCR, Ab-TCR, AABD-TCR, αβTFP, γδTFP or a TCR with TCRγ and TCRδ constant chains.

The disclosure also provides novel deletion mutants of the constant chains of TCRα (SEQ ID Nos (DNA): 7172-7271; SEQ ID Nos (PRT): 7863-7963), TCRβ (SEQ ID NO (DNA): 7273-7398; SEQ ID NO: (PRT): 7965-8090), TCRγ (SEQ ID NO (DNA): 7400-7499; SEQ ID NO (PRT):8092-8191) and TCRδ (SEQ ID NO (DNA): 7501-7600; SEQ ID NO (PRT):8193-8292) (Table 45) that can be used in the construction of SIRs and cTCR and SARs based on the SIR and cTCR backbones. Use of the deletion mutants of the constant chains of TCRα, TCRβ, TCRγ and TCRδ help to reduce the size of the SIR/SAR constructs, improve their packaging into viral vectors and thereby improve viral vector titer and transduction efficiency. The deletion mutants of the constant chains of TCRα, TCRβ (β1 or β2), TCRγ and TCRδ chains described here can be used to constructs SARs with diverse expression, binding affinity and activity as compared to SARs composed of full-length constant chains. For example, the deletion mutants of the constant chains of TCRα, TCRβ (β1 or β2), TCRγ and TCRδ chains described here can be used to constructs SARs with increased expression, binding affinity, signaling activity, cytokine production and/or cytotoxicity as compared to SARs composed of full-length constant chains. Alternatively, the deletion mutants of the constant chains of TCRα, TCRβ (β1 or β2), TCRγ and TCRδ chains described here can be used to constructs SARs with decreased expression, binding affinity, signaling activity, cytokine production and/or cytotoxicity as compared to SARs composed of full-length constant chains. The SAR constructs with increased expression, binding affinity, signaling activity, cytokine production and/or cytotoxicity as compared to SARs composed of full-length constant chains may be useful to target diseased cells (e.g., tumor cells) with low level expression of the target antigens. The SAR constructs with decreased expression, binding affinity, signaling activity, cytokine production and/or cytotoxicity as compared to SARs composed of full-length constant chains may be useful to selectively target tumor cells with high level expression of the target antigen(s) while sparing normal healthy cells expressing low level expression of the target antigens.

In one aspect, the disclosure describes a double chain SAR where the TCRα constant chain fragment is less than 370, 380, 390, 400, 410 or 421 nucleotides in length and TCRβ constant chain fragment is less than 490 nucleotides, less than 500 nucleotides, less than 510 nucleotides, less than 520 nucleotides, less than 530 nucleotides or less than 540 nucleotides in length. In one aspect the SAR has the backbone of a SIR, cTCR, Ab-TCR, AABD-TCR, αβTFP, or a TCR. In one aspect the SAR has the backbone of a SIR. In one aspect the SAR has the backbone of a SIR with TCRα and TCRβ constant chains or TCRγ and TCRδ constant chains. In one aspect the SAR has the backbone of a cTCR with TCRα and TCRβ constant chains or TCRγ and TCRδ constant chains. In one aspect the TCRα and TCRβ constant chain fragments carry mutations that enhance their chain-pairing and reduce chain pairing with the endogenous TCRαβ chains. In one aspect the TCRα and TCRβ constant chain fragments carry mutations that result in an extra cysteine bond (double bond) between the two chains.

In one aspect the disclosure provides SARs comprising a TCRα constant chain deletion mutant selected from any of TCRα constant chains represented by SEQ ID NO: 7864-7963 or a variant with at least 70%, 75%, 80%, 85%, 90%, 95%, 98%, 99%, 99.9% homology to the amino acid sequence represented by SEQ ID NO: 7864-7963. In one aspect the disclosure provides SARs comprising a TCRα constant chain fragments comprising any of the SEQ ID NO: 7864-7963 or their deletion mutants or functional variants which retains the ability to pair with the complementary TCRβ constant chain. In one aspect the disclosure provides SARs comprising a TCRα constant chain fragment comprising any of the SEQ ID NO: 7864-7963 or their deletion mutants or functional variants which retains the ability to incorporate into the TCR/CD3 complex, recruit a TCR signaling module and/or induce T cell signaling upon engagement of target antigen. Additional TCRα constant chain deletion mutants and functional variants that can be used in the construction of the SARs.

In one aspect the disclosure provides SARs comprising a TCRβ constant chain deletion mutant selected from any of TCRβ constant chains represented by SEQ ID NO: 7965-8090 or a variant with at least 70%, 75%, 80%, 85%, 90%, 95%, 98%, 99%, 99.9% homology to the amino acid sequence represented by SEQ ID NO: 7965-8090. In one aspect the disclosure provides SARs comprising a TCRβ constant chain fragments comprising any of the SEQ ID NO: 7965-8090 or their deletion mutants or functional variants which retains the ability to pair with the complementary TCRα constant chain. In one aspect the disclosure provides SARs comprising a TCRβ constant chain fragment comprising any of the SEQ ID NO: 7965-8090 or their deletion mutants or functional variants which retains the ability to incorporate into the TCR/CD3 complex, recruit a TCR signaling module and/or induce T cell signaling upon engagement of target antigen. Additional TCRβ constant chain deletion mutants and functional variants that can be used in the construction of the SARs.

In one aspect the disclosure provides SARs comprising a TCRγ constant chain deletion mutant selected from any of TCRγ constant chains represented by SEQ ID NO: 8092-8191 or a variant with at least 70%, 75%, 80%, 85%, 90%, 95%, 98%, 99%, 99.9% homology to the amino acid sequence represented by SEQ ID NO: 8092-8191. In one aspect the disclosure provides SARs comprising a TCRγ constant chain fragments comprising any of the SEQ ID NO: 8092-8191 or their deletion mutants or functional variants which retains the ability to pair with the complementary TCRS constant chain. In one aspect the disclosure provides SARs comprising a TCRγ constant chain fragment comprising any of the SEQ ID NO: 8092-8191 or their deletion mutants or functional variants which retains the ability to incorporate into the TCR/CD3 complex, recruit a TCR signaling module and/or induce T cell signaling upon engagement of target antigen. Additional TCRγ constant chain deletion mutants and functional variants that can be used in the construction of the SARs.

In one aspect the disclosure provides SARs comprising a TCRS constant chain deletion mutant selected from any of TCRS constant chains represented by SEQ ID NO: 8193-8292 or a variant with at least 70%, 75%, 80%, 85%, 90%, 95%, 98%, 99%, 99.9% homology to the amino acid sequence represented by SEQ ID NO: 8193-8292. In one aspect the disclosure provides SARs comprising a TCRS constant chain fragments comprising any of the SEQ ID NO: 8193-8292 or their deletion mutants or functional variants which retains the ability to pair with the complementary TCRγ constant chain. In one aspect the disclosure provides SARs comprising a TCRS constant chain fragment comprising any of the SEQ ID NO: 8193-8292 or their deletion mutants or functional variants which retains the ability to incorporate into the TCR/CD3 complex, recruit a TCR signaling module and/or induce T cell signaling upon engagement of target antigen. Additional TCRS constant chain deletion mutants and functional variants that can be used in the construction of the SARs.

In some embodiments of any of the SARs described herein, the heterologous antigen-binding domain is selected from the group of: an antibody, an antibody fragment (vL, vH, Fab etc.) a scFv, a (scFv)2, a VHH domain, FHVH (a fully human vH domain), a single domain antibody, a non-immunoglobulin antigen binding scaffold (e.g., Centyrin, affibody, ZIP domain, an adaptor etc.), a VNAR domain, a ligand, a TCR, variable domain (Va, Vb, Vg, Vd) of a TCR and a receptor. In some embodiments of any of the SARs described herein, the heterologous antigen-binding domain comprises a scFv.

The antigen binding domain of SAR of the disclosure may an HLA-independent TCR, a single domain TCR, a ligand binding domain of a receptor, a receptor binding domain of a ligand, a non-immunoglobulin antigen binding scaffold, an adaptor or a fragment thereof.

In one aspect, the disclosure provides novel compositions of synthetic antigen receptor (SARs). In another aspect, the disclosure provides novel configuration/architectures of SARs. In another aspect, the disclosure provides SARs with useful biological properties (e.g., expression, binding affinity, effector functions etc.). In another aspect, the disclosure provides SARs capable of binding to one or more than one antigen. In another aspect, the disclosure provides SARs capable of binding to one or more than one epitope of an antigen.

In one aspect, the disclosure provides a synthetic antigen receptor (SAR) comprising more than one (i.e., 2, 3, 4, 5 or more) antigen binding domains. In another aspect, the disclosure provides a SAR capable of binding to and/or responding to more than one antigen or more than one epitope of an antigen. In another aspect, the disclosure provides a bispecific and/or a multispecific SAR capable of binding to and/or responding to more than one antigen or more than one epitope of an antigen. In another aspect, the disclosure provides useful antigen binding domains for construction of a bispecific and/or a multispecific SAR. In another aspect, the disclosure provides useful configurations (i.e., the location of different domains) for a bispecific and/or a multispecific SAR. The bispecific and multispecific SAR of disclosure when expressed in an immune effector cell (e.g., a T cell, NKT cell or NK cell etc.) confers on it the ability to bind to and/or respond to more than one antigen or more than one epitope of an antigen with nearly equal efficacy or greater efficacy as compared to two or more unispecific SAR targeting those same antigens or same epitopes of those antigens.

The presence of two or more antigen binding domains in a bispecific or multi-specific SAR may result in steric hinderance, non-specific aggregation, poor expression, protein unfolding, and/or interference with antigen binding. In addition, the location of the antigen binding domain(s) relative to the transmembrane domain of SARs needs to be optimized in order to optimize signal transduction by the resulting receptor. Bispecific and multispecific CARs incorporating two or more scFv have been described in the art. However, the disclosure identifies that presence of more than one scFv (i.e., 2, 3, 4 or more) in a SAR often results in steric hinderance, non-specific aggregation, tonic-signaling, poor expression, protein unfolding, and/or interference with antigen binding resulting in poor signaling and effector function (e.g., cytokine production, cytotoxicity etc.). Therefore, a major challenge in the generation of bispecific and multi-specific SARs comprising two or more antigen binding domains is to determine useful antigen binding domains (e.g., scFv, Fv, Fab, vHH, FHVH, Centyrin, affibody, cytokine, receptor, svd-TCR, etc.) that should be incorporated in such SARs so as to reduce steric hinderance, non-specific aggregation, tonic-signaling, poor expression, protein unfolding, and/or interference with antigen binding that can lead to poor signaling and effector function (e.g., cytokine production, cytotoxicity etc.).

A second challenge is to determine a useful configuration of the various antigen binding domains that comprise the bispecific and multi-specific SARs. For example, the optimal order of various antigen binding domains with respect to each other and with respect to other components of the SAR (e.g., hinge domain, transmembrane domain etc.) needs to be determined to reduce non-specific aggregation, tonic-signaling, poor expression, protein unfolding, and/or interference with antigen binding resulting in poor signaling and effector function (e.g., cytokine production, cytotoxicity etc.). This is a significant challenge for all SARs and particularly for multichain SARs, such as those described in this disclosure (e.g., double chain CD16 SAR, double chain Dap10 SAR, double chain NKp30 SAR etc.) whose antigen binding domain is composed of two different fragments (e.g., vL and vH, Va and Vb or Vg and Vd etc.). For example, the attachment of a second antigen binding domain (e.g., scFv or a vHH domain) to a double chain SAR which binds to CD19 through a vL and vH fragments that are operably linked to two separate CD16 chains but join to form a Fv that binds to CD19 could potentially interfere with the interaction between the vL and vH fragments resulting in their inability to form a functional Fv that can bind CD19.

The length of the hinge domain, which determines the distance between the antigen binding domain and the cell membrane, may influence the signaling via a chimeric antigen receptor. Therefore, another challenge in the field is that it is not known at the present whether attachment of multiple antigen binding domains may adversely affect the formation of an effective immunological synapse and signaling via a SAR by increasing the distance between the target antigen and the cell membrane.

Fusion of multiple antigen binding domains in a SAR could result in steric hinderance and improper folding. Another challenge in the field is that it is not known at the present whether linker domains are needed between the different antigen binding domains of a bispecific/multispecific SAR. The length and nature of the linker domains is also not known. This is of particular importance in case of double chain SAR (e.g., double chain CD16 SAR, double chain NKp30 SAR, double chain NKp44 SAR, double chain Dap10 SAR etc.) as the addition of an improper linker could potentially interfere with the interaction between the two chains or formation of a functional Fv. Additionally, linker(s) could adversely affect the formation of an effective immunological synapse and signaling via a SAR by increasing the distance between the target antigen and the cell membrane.

In one aspect, the disclosure offers solution to the above problems.

In one aspect, the disclosure provides SARs with one or more antigen binding domains and one or more transmembrane domains. In an embodiment, the disclosure provides useful antigen binding domains for construction of bispecific and multispecific SARs.

The disclosure provides several exemplary SARs comprising different antigen binding domains, hinge domains, linker domains, connecting peptides, transmembrane domains, activation domains, costimulatory domains, accessory modules and therapeutic controls etc. The nucleic acid and amino acid sequences of several exemplary SARs of the disclosure are provided in SEQ ID NO (DNA): 1600-2328, 4851-5129, 5451-6282, 7160-7170, 7601-7747, 8768-9602, 10817-10830 and SEQ ID NO (PRT): 3994-4722, 5151-5429, 6283-7114, 7852-7862, 8293-8439, 9860-10694. The names and configuration of the exemplary SARs of the disclosure are provided in Tables 32-34 and 36-42 of the provisional application which is incorporated in its entirety by reference herein. The SEQ ID (DNA) and SEQ ID (PRT) of exemplary components that can be used in the construction of the SAR are provided in SEQ ID NO (DNA):31-1243, 1308-1572 and 8535 to 8767 and SEQ ID NO (PRT):2425-3637, 3702-3966 and 9627-9859, 10832-10841, and 12304-12311. The names of the different SAR components and accessory reagents that can be used in the construction of SARs of the disclosure are provided in Tables 1-31 of the provisional application which is incorporated in its entirety by reference herein. The target antigen(s), configuration and composition of the SARs can be deduced from their nucleic acid sequences and amino-acid sequences provided in this disclosure by performing sequence homology search for their component modules using programs such as BLAST. Alternatively, softwares, such as ApE ([https://]jorgensen.biology.utah.edu/wayned/ape/), can be used to determine the composition of the different SAR constructs whose nucleic acid sequences are provided in this disclosure. Finally, the configuration and composition of the different SARs of the disclosure can be deduced from their names by those skilled in the art.

In an embodiment, the disclosure provides a novel SAR with the architecture and/or configuration represented by any of the exemplary SARs provided herein. In an embodiment, the disclosure provides a novel SAR with the composition of any of the exemplary SARs provided herein or a functional variant thereof. In an embodiment, the disclosure provides a novel SAR with at least 70% homology (e.g., 70%, 75%, 80%, 85%, 90%, 95%, 98%, 99% homology) to the amino acid sequence of any of the exemplary SARs provided herein. In an embodiment, the disclosure provides a novel SAR with at least 70% homology (e.g., 70%, 75%, 80%, 85%, 90%, 95%, 98%, 99% homology) to the amino acid sequence of any of the exemplary SARs provided herein excluding the optional accessory modules. In an embodiment, the disclosure provides a novel SAR with at least 70% homology (e.g., 70%, 75%, 80%, 85%, 90%, 95%, 98%, 99% homology) to the amino acid sequence of any of the exemplary SARs provided herein in the regions comprising their antigen binding domain(s) and signaling chain(s) (e.g., CD16 chains).

In one aspect, the disclosure relates to the use of autonomous antigen binding domains (AABD) including human VH domains, typically multiple human VH domains, as building blocks to make SARs with advantageous antigen binding domains.

The disclosure relates in one aspect to an autonomous antigen binding domain (AABD), methods of generating the same and uses of such domains for construction of synthetic antigen receptors and potentially antibody therapeutics. In one embodiment, the AABD domain has improved stability. In another embodiment, the AABD domain has improved thermal stability. In another embodiment, the AABD domain has improved solubility. In another embodiment, the AABD domain has less tendency for self-aggregation. In another embodiment, the AABD domain has improved ability to be secreted in the extracellular space when expressed in a mammalian cell with an N-terminal signal peptide.

In one aspect the AABD is a single domain antibody or antibody fragment. In one aspect, an AABD is a single variable heavy chain (VH or vH) domain (SVH domain) or a fragment thereof that is capable of binding the antigen in the absence of a variable light chain (VL or vL) domain. In another aspect, the AABD is a single variable heavy chain (VH) domain (or a SVH domain) of a fragment thereof that can be expressed as a soluble protein in the absence of a vL domain. In another aspect, the AABD is a single variable heavy chain (VH) domain (or a SVH domain) or a fragment thereof that can be expressed as a secreted protein in the absence of a vL domain when joined to an N-terminal secretory signal. Certain embodiments of disclosure relate to a SAR comprising a first AABD, the first AABD specifically binding to an antigen in the absence of a second domain.

In one aspect the AABD is a single variable light chain (VL or vL) domain or a SVL domain or a fragment thereof that is capable of binding the antigen in the absence of a variable heavy chain (VH or vH) domain. In another aspect, the AABD is a single variable light chain (VL) domain (or a SVL domain) or a fragment thereof that can be expressed as a soluble protein in the absence of a vH domain. In another aspect, the AABD is a single variable light chain (VL) domain (or a SVL domain) or a fragment thereof that can be expressed as a secreted protein in the absence of a vH domain when joined to an N-terminal secretory signal.

In some embodiment, an AABD is a non-scFv based antigen binding domain, a camelid vHH domain, a humanized vHH domain, a non-immunoglobulin antigen binding scaffold, the receptor binding domain of cytokine or a ligand, the ligand binding domain of a receptor, a single variable domain T cell receptor (TCR), an autoantigen or a fragment thereof.

In an embodiment, an AABD is an adaptor domain, an adaptor binding domain or a fragment thereof. Exemplary adaptors and adaptor binding domain include but are not limited to: RZIP, EZIP, E4, K4, NKG2D-YA, NKG2D-AF, D domains and the like.

The terms “single domain antibody, variable single domain or immunoglobulin single variable domain (ISV)” are all well known in the art and describe the single variable fragment of an antibody that binds to a target antigen. These terms are used interchangeably herein. As explained below, embodiments of the various aspects of the disclosure relate to SARs comprising single heavy chain variable domain antibodies/immunoglobulin heavy chain single variable domains, designated SVH domains, which bind to different antigens, such as CD19, CD20, CD22, BCMA, CD3ε, MPL, CD123, CD33, Mesothelin, Her2, CS1/SLAMF7, CD30, GD2, GD3, FLT3, ROR1, CD79b, Lym1, Lym2, PSCA, PSMA, ALK, CD138, CEA, FAP, TAJ, CD229, IL13Ra2, CD32b, GPC3, Muc16 and KIR3DL2 in the absence of light chain. Human heavy chain single variable domain antibodies are typically used.

Thus, in some embodiments, the SARs of the disclosure comprise a binding domain that comprises or consist of a single domain antibody wherein said domain is a single human heavy chain variable domain (SVH). Thus, in some aspect, the SARs of the disclosure comprise one or more binding domain that is devoid of VL domains.

Thus, in some embodiments, the SARs of the disclosure comprise a binding domain that comprises or consist of a single domain antibody wherein said domain is a camelid vHH (or VHH) domain or humanized vHH domain.

As used herein, the VH domain is a human VH domain or a non-human VH domain.

The SVH domains are small molecules of 12-14 kDa which can be combined into different formats to give multivalent or multispecific antigen binding domains of a SAR. SVH domains are robust and are characterized by high affinity and stability in serum. SVH domains are also characterized by high solubility in serum and lack of aggregation.

Each single VH domain (SVH) antibody comprises three CDRs and four FRs, arranged from amino-terminus to carboxy-terminus in the following order: FR1-CDR1-FR2-CDR2-FR3-CDR3-FR4. Thus, in one embodiment of the disclosure, the domain is a human variable heavy chain (VH) domain with the following formula FR1-CDR1-FR2-CDR2-FR3-CDR3-FR4.

In an embodiment, the disclosure provides single-chain and multichain SARs (e.g., CD16, NKp30, NKp44, NKp46, Dap10 etc.) that can be constructed using SVH domains having a W103R substitution according to Kabat system. An exemplary SVH targeting CD19 with W103R substitution is CD19-FHVH-354 and is represented by SEQ ID NO (DNA): 836 and SEQ ID NO (PRT): 3230. In another embodiment, the disclosure provides multichain SARs having bispecific, bivalent or biparatopic antigen binding moieties that comprise SVH domains having a W103R substitution according to Kabat system.

In another embodiment, the disclosure provides multichain SARs having multispecific, multivalent or multiparatopic antigen binding moieties that comprise SVH domains having a W103R substitution according to Kabat system. As AABD are modular in nature, an AABD can be substituted by other AABD targeting different antigens to develop SARs targeting those antigens.

In another embodiment, the disclosure provides single chain SARs having bispecific, bivalent or biparatopic antigen binding moieties that comprise SVH having a W103R substitution according to Kabat system.

In an embodiment, the disclosure provides that single chain and multi-chain SARs can be constructed using SVH stabilized by the introduction of non-canonical cysteines, which are capable of forming disulfide bonds and/or form disulfide bridges under suitable conditions. An exemplary SVH comprising non-canonical cysteins is CEA-300-aVH and is provided in SEQ ID NO (DNA): 954 and SEQ ID NO (PRT): 3348. Additional exemplary such SVH are provided in WO2019149715, which is incorporated in its entirety by reference herein.

In one embodiment, the disclosure is aimed at mitigating the shortcomings of existing adoptive cellular therapies by providing single chain SARs comprising SVH where the SVH domains contains the substitution cysteines in positions (i) 52a and 71 or (ii) 33 and 52 according to Kabat numbering, wherein said cysteines are capable of forming a disulfide bond and/or form a disulfide bond under suitable conditions.

In an embodiment of the disclosure, the SVH domain used to make a SAR comprises a substitution selected from the group consisting of 44E, 45E, 45 R, (101-1)Y and 101D according to Kabat numbering. Particularly, the SVH comprises the substitutions 44E, 45E or 45R, (101-1)Y and 101D according to Kabat numbering. In an embodiment, the SVH domain comprises a substitution selected from the group consisting of G44E, T45E, T45 R, F(101-1)Y and A101D according to Kabat numbering. In an embodiment, the SVH domain comprises the substitutions G44E, T45E, T45 R, F(101-1)Y and A101D according to Kabat numbering.

In an embodiment of the disclosure, the SAR comprises an SVH with substitution selected from the group consisting of 44E, 45E and (101-1)Y according to Kabat numbering. In an embodiment, the SAR comprises an SVH domain with the substitutions 44E, 45E, and (101-1)Y according to Kabat numbering. In an embodiment, the SVH domain comprises a substitution selected from the group consisting of G44E, T45E and F(101-1)Y according to Kabat numbering, if present in the SVH domain. In an embodiment, the SAR comprises an SVH domain comprising the substitutions G44E, T45E, and F(101-1)Y according to Kabat numbering.

In an embodiment, the SVH domain of the SAR comprises a vH framework comprising a FR1, FR2, FR3 and FR4 with at least 85% sequence identity to the amino acid sequence of SEQ ID NOs: 21411, 21412, 21413 and 21414, respectively.

In an embodiment, the SVH domain of the SAR comprises a vH framework comprising a FR1, FR2, FR3 and FR4 with at least 85% sequence identity to the amino acid sequence of SEQ ID NO: 4819-4822, respectively.

In an embodiment, the SVH domain of the SAR comprises a vH framework comprising a FR1, FR2, FR3 and FR4 with at least 85% sequence identity to the amino acid sequence of SEQ ID NO: 4823-4826, respectively.

In an embodiment of the disclosure, the SVH domain of the SAR comprises a vH framework comprising a FR1, FR2, FR3 and FR4 with at least 85% sequence identity to the amino acid sequence of SEQ ID NO: 4827-4830, respectively.

In an embodiment of the disclosure, the SVH domain comprises a vH framework comprising a FR1, FR2, FR3 and FR4 with at least 85% sequence identity to the amino acid sequence of SEQ ID NO: 4831-4834, respectively.

In an embodiment of the disclosure, the SVH domain comprises a vH framework comprising a FR1, FR2, FR3 and FR4 with at least 85% sequence identity to the amino acid sequence of SEQ ID NO: 4835-4838, respectively.

In an embodiment, the SVH domain comprises a vH framework comprising a FR1, FR2, FR3 and FR4 with at least 85% sequence identity to the amino acid sequence of SEQ ID NO: 4839-4842, respectively.

In an embodiment, the SVH domain comprises a vH framework comprising a FR1, FR2, FR3 and FR4 with at least 85% sequence identity to the amino acid sequence of SEQ ID NO: 4843-4846, respectively.

The SVH domain is particularly useful for construction of a SAR, as FRI-4 according to SEQ ID NOs 4819-4650 are not immunogenic in humans.

In some embodiments, the SAR constructs described herein include a human SVL domain (typically multiple human SVL domains) that recognizes a target protein of interest, e.g., a protein expressed on a tumor cell, such as an antigen. The term SVL domain as used herein refers to a single human VL domain antibody (VL sdAb). These terms are thus used interchangeably. The term SVL is also used interchangeably with independent vL domains or autonomous vL domains. An SVL is a type of AABD.

In one aspect, the AABD of a SAR is a camelid vHH domain. The disclosure also relates to a SAR comprising multiple vHH domains. The disclosure also relates to a SAR comprising humanized vHH domains. Exemplary vHH domains that can be used in the construction of the SAR of the disclosure and their target antigens are presented in Table 5.

In one aspect, the AABD of a SAR is a non-immunoglobulin antigen binding scaffold, such as DARPIN, an affibody, an affilin, an adnectin, an affitin, an obodies, a repebody, a fynomer, an alphabody, an avimer, an atrimer, a centyrin, a pronectin, an anticalin, a kunitz domain, an Armadillo repeat protein, D domain, or a fragment thereof. The disclosure also relates to a SAR comprising multiple non-immunoglobulin antigen binding scaffold. Exemplary non-immunoglobulin antigen binding scaffold that can be used in the construction of the SARs of the disclosure and their target antigens are presented in Table 7-9.

In one aspect, the AABD of a SAR is an adaptor binding domain (e.g., RZIP, EZIP, E4, K4, NKG2D-AF, NKG2D-YA, or D domain etc.). The disclosure also includes SARs that bind to multiple adaptors. In an embodiment, an adaptor binding domain is a leucine zipper domain. In one aspect, the AABD of a SAR binds to an adaptor (e.g., RZIP, EZIP, E4, K4, D domain, Streptag, FITC, Biotin, ULBP2R, ULBP2-S3 etc.). It is understood by those skilled in the art that an adaptor and an adaptor binding protein can be substituted for each other. Thus, a SAR can comprise an RZIP module that binds to a SAR adaptor comprising an EZIP module. Alternatively, the SAR may comprise an EZIP module while the SAR adaptor may comprise an RZIP module. The disclosure also includes SARs that bind to multiple adaptors.

In one aspect, the AABD of a SAR is the extracellular ligand-binding domain of a receptor or a fragment thereof. The disclosure also includes SARs that comprise multiple extracellular ligand binding domains of receptors.

In one aspect, the AABD of a SAR is the extracellular receptor-binding domain of a ligand or a cytokine or a fragment thereof. The disclosure also includes SARs that comprise multiple extracellular receptor binding domains of ligands or cytokines.

In one aspect, the AABD of a SAR is an autoantigen. The disclosure also includes SARs that comprise multiple auto-antigens. An exemplary auto-antigen that can be used in the construction of a SAR is Dsg3 or a fragment thereof.

In one aspect, the AABD of a SAR is a single variable domain of a T cell receptor (svd-TCR). The disclosure also includes SARs that comprise multiple single variable domains of T cell receptors. Exemplary polynucleotides comprising svd-TCR domains are provided in SEQ ID NO (DNA): 21563-21564 of PCT/US2021/022641 and in WO2021030182 which are incorporated in its entirety by reference herein.

In one aspect, the AABD of a SAR is any single domain protein that can bind to an antigen expressed on the surface of a cell.

The multiple AABD in a SAR could be present in different combinations (e.g., two Centyrins, one Centyrin and one vHH domain, vHH domain and a SVH domain and a Centyrin etc.)

In one aspect, the AABD of a SAR is a Centyrin. The disclosure also relates to a SAR comprising multiple Centyrins. In one aspect, the AABD of a SAR is a DARPINS. The disclosure also relates to a SAR comprising multiple DARPINs. Similarly, the disclosure relates to SARs containing multiple non-immunoglobulin antigen binding domains such as affibodies, affilins, adnectins, affitins, obodies, repebodies, fynomers, alphabodies, avimers, atrimers, pronectins, anticalins, kunitz domains, Armadillo repeat proteins.

In some embodiments, the SAR contains multiple AABDs. In an embodiment, the first AABD is linked to a second AABD, wherein the first and second AABD specifically bind antigens. In an embodiment, the antigens recognized by the SAR are peptide antigens that are bound to MHC complex. In some embodiments, the two or more AABD of a SAR recognize the same antigen. In other embodiments, the two or more AABD of the SAR recognize different antigens.

Whilst scFv typically used in SARs, such as 2nd generation CARs, have the potential for unwanted aggregation, cluster formation and immunogenicity, the use of Autonomous Antigen Binding Domains (AABD) provides a stable format with substantially reduced potential for immunogenicity, non-specific-aggregation or unfolding. This is particularly useful when designing SARs having bispecific, bivalent or biparatopic antigen binding moieties. As demonstrated by the inventors herein, multiple AABD domains can readily be used in such multimeric format thus facilitating the generation of multispecific SARs that enable simultaneous targeting of more than one target antigen or more than one epitope of an antigen.

In an embodiment, the disclosure provides a SAR that can target one or more than 1 antigen (e.g., 1, 2, 3, 4, 5, 6 or more antigens). In an embodiment, the disclosure provides a SAR that can target one or more than 1 epitope (e.g., 1, 2, 3, 4, 5, 6 or more epitopes). In an embodiment, the disclosure provides a SAR that comprise one or more than 1 antigen binding domains (e.g., 1, 2, 3, 4, 5, 6 or more antigen-binding domains).

In an embodiment, the disclosure provides a SAR that comprises one or more than one chain with each chain comprising zero, one or more antigen binding domains operably linked to a transmembrane domain, an optional activation domain and an optional co-stimulatory domain. In an embodiment, the activation domain encodes for one or more ITAM motifs.

In an embodiment, the disclosure provides a synthetic antigen receptor, comprising (a) one or more antigen-specific targeting regions, (b) at least one extracellular linker domain, (c) at least one transmembrane domain, (d) an optional co-stimulatory domain and (e) an optional intracellular signaling domain, wherein one antigen-specific targeting region comprises an antigen-specific single chain Fv (scFv) fragment, and a second antigen specific targeting domain comprises an AABD. In an exemplary embodiment, the AABD is a non-scFv antigen binding module (e.g., a SVH, vHH, FHVH, SVL, svd-TCR, Centyrin, DARPIN, CD16A, CD64, CD32, NKG2D, NKG2D-AF, NKG2D-YA, RZIP, EZIP, E4, K4, D domain etc.).

In an embodiment, the disclosure provides a bispecific or a multispecific synthetic antigen receptor comprising (a) at least two antigen-specific targeting regions, (b) at least one extracellular linker domain, (c) at least one transmembrane domain, (d) an optional co-stimulatory domain and (e) an optional intracellular signaling domain, wherein one antigen-specific targeting region comprises an antigen-specific single chain Fv (scFv) fragment, and a second antigen specific targeting domain comprises an AABD. In an exemplary embodiment, the AABD is a non-scFv antigen binding module (e.g., a SVH, vHH, FHVH, SVL, svd-TCR, Centyrin, DARPIN, CD16A, CD64, CD32, NKG2D, NKG2D-AF, NKG2D-YA, RZIP, EZIP, E4, K4, D domain etc.). An exemplary bispecific SAR comprising two AABD and targeting CD38 and BCMA is CD8SP-CD38-717-vHH-Ecoil-BCMA-346-vHH-CD16A-F158V-S197P-FL-v3 (SEQ ID NO (DNA): 5100; SEQ ID NO (PRT):5400) In an embodiment, the disclosure provides a bispecific or a multispecific synthetic antigen receptor having the general formula: (AABD)n-optional linker domain-scFv-hinge domain-transmembrane domain-optional one or more costimulatory domains—an activation domain; where n=0, 1, 2, 3, 4, 5 or more and where the activation domain may contain one or more ITAM motifs. An exemplary such SAR is represented by IgSP-Apa-CD20-USC1-vHH-2HCD26-G4Sx3v2-hu-mROO5-1-scFv-CD16-F158V-S197P-FL-TMCP-v3 (SEQ ID NO (DNA): 7160 and SEQ ID NO (PRT): 7853). This SAR has one antigen binding domain represented by humanized hu-mROO5-1 scFv that targets CD19 and a second antigen binding domain represented by CD20-vHH-2HCD26 that targets CD20. The two antigen binding domains are linked via a Gly-Ser (G4Sx3v2) flexible linker. This SAR construct also comprise a CD16 extracellular domain (including CD16 D1 and D2 domains), a CD16 hinge and transmembrane domains and a CD16 cytosolic domain. Other exemplary bispecific SARs are represented by SEQ ID NO:7161-7170.

Another exemplary bispecific SAR is represented by IgSP-Apa-CD20-USC1-vHH-2HCD26-G4Sx3v2-hu-mROO5-1-scFv-CD28-Hinge-CD16-F158V-S197P-Hinge-TM-CP-v3 (SEQ ID NO: 7164). This construct is similar to the construct with SEQ ID NO: 7160 described above except it lacks the CD16 D1 and D2 domain and comprise a CD28 hinge domain.

In an embodiment, the disclosure provides a SAR that comprises one or more than one chain with each chain comprising zero, one, two or more antigen binding domains operably linked to a transmembrane domain but lacking an activation domain. Although such a SAR lacks an activation domain of its own, it is capable of signal transduction by recruitment of a signaling module comprising protein(s) that encode an activation domain. Examples of signaling proteins can be recruited by such a SAR include CD3z, DAP10 or DAP12. An exemplary such a SAR is based on the backbone of a CD16 SAR, NKp30 SAR, NKp44 SAR or NKp46 SAR. In an exemplary embodiment, the disclosure provides a SAR in which one or more AABD are attached to the N-terminus or near the N-terminus of one or both chains of a double chain SAR. In an exemplary embodiment, the disclosure provides a SAR in which one or more AABD are attached to the N-terminus or near the N-terminus of the vL or vH fragments comprising one or both chains of a SAR. In an exemplary embodiment, the disclosure provides a SAR in which one or more AABD are attached to the N-terminus or near the N-terminus of the Va, Vb, Vg or Vd fragments comprising one or both chains of a TCR. In an exemplary embodiment, the AABD is a non-scFv antigen binding module (e.g., a SVH, vHH, FHVH, SVL, svd-TCR, Centyrin, DARPIN, CD16A, CD64, CD32, NKG2D, NKG2D-AF, NKG2D-YA, RZIP, EZIP, E4, K4, D domain etc.).

In an embodiment, the disclosure provides a one and a half chain SAR or a double chain SAR comprising one chain that has the general formula: (AABD)n-optional linker domain-scFv-optional linker-TCR constant chain; and the second chain that has the general formula (AABD)n-optional linker domain-CD16/NKp30/NKp44/Nkp46 constant chain where n=0, 1, 2, 3, 4, 5 or more. The CD16/NKp30/NKp44/Nkp46 constant chain comprise the full-length CD16/NKp30/NKp44/Nkp46 polypeptide or fragment or a mutant or a variant thereof that is either capable of directly transmitting a signal to an immune cell (e.g., T cell or an NK cell etc.) or is capable of recruiting one or more signaling proteins that are capable of transmitting a signal to an immune cell. The transmitted signal may comprise a signal to stimulate cell proliferation, activation, cytokine secretion and/or cytotoxicity.

In an embodiment, the disclosure provides a double chain bispecific synthetic antigen receptor comprising two chains, each chain comprising (a) one or more heterologous antigen-specific targeting regions, (b) at least one extracellular linker domain, (c) at least one transmembrane domain, (d) an optional co-stimulatory domain and (e) an optional intracellular signaling domain, wherein one antigen-specific targeting region comprises a vL and/or a vH fragment that is capable of combining with the vH and/or vL fragment present on the second chain to create a fragment variable (Fv), and the second antigen specific targeting domain comprises an AABD (e.g., a vHH, SVH, Centyrin, affibody etc.). In one embodiment, the Fv binds an antigen. In another embodiment, the Fv does not bind an antigen. In an embodiment, the Fv serves as the scaffold for the attachment of the second antigen specific targeting domain comprising an AABD. In an embodiment, the AABD is a non-scFv antigen binding domain.

An exemplary double chain bispecific SAR comprising two chains is CD8SP-CD20-VHH-2HC2D6-USC1-Kpn-G4S-EcoR1-hu-mROO5-1-vL-xho-IgCL-Bam-NKp30-ECDTMCP-opt1-F-P2A-SP-hu-mROO5-1-vH-Mlu-IgG1-CH1-Kpn-NKp30-ECDTMCP-opt2-F-F2A-PAC and is represented by SEQ ID NO (DNA): 1605 and SEQ ID NO (PRT): 3999. One chain of the SAR construct comprises the humanized hu-mROO5-1 vL fragment fused to NKp30 extracellular, transmembrane and cytosolic domains via an IgCL linker and the other chain of the SAR comprises the humanized hu-mROO5-1 vH fragment fused to a second chain comprising NKp30 extracellular, transmembrane and cytosolic domains. Via the IgG1-CH1 linker. The hu-mROO5-1 vL and hu-mROO5-1 vH fragments of the SAR together form a Fv targeting CD19. A vHH fragment targeting CD20 (CD20-USC1-vHH-2HCD26; SEQ ID NO: 841) is fused to the N-terminus of the hu-mROO5-1 vH fragment via a Glycine-Serine linker. Thus, the SAR targets CD19 via the hu-mROO5-1 Fv and targets CD20 via CD20-USC1-vHH-2HCD26. It is to be noted that SARs are modular in format. Therefore, it is possible to replace one module of a SAR with another module. For example, the hu-mROO5-1 vL and hu-mROO5-1 vH fragments can be replaced with vL/vH fragment targeting a different antigen. Similarly, the CD20-USC1-vHH-2HCD26 module can replaced by another AABD targeting a different antigen. The IgCL and IgG1-CH1 linker can be replaced by other suitable Ig like linkers provided in SEQ ID NO: 1142-1175 (Table 13). Finally, one of both of the NKp30 fragments can be replaced by one or both polypeptides derived fromNKp44, NKp46, CD16, CD3z or DAP10.

An exemplary construct in which one of the NKp30 ECDTMCP chains is replaced by a CD16-F158V-S197P-FL-TMCP chain is represented by CD8SP-CD20-VHH-2HC2D6-USC1-Kpn-G4S-EcoR1-hu-mROO5-1-vL-xho-IgCL-Bam-NKp30-ECDTMCP-opt1-F-P2A-SP-hu-mROO5-1-vH-Mlu-IgG1-CH1-Kpn-CD16-F158V-S197P-FL-TMCP-v3-F-F2A-PAC (SEQ ID NO (DNA):1618 an SEQ ID NO (PRT): 4012). Other exemplary unispecific, bispecific and trispecific SAR constructs are provided in Table 32 of provisional application.

An exemplary trispecific double chain construct targeting CD19, CD20 and BCMA is represented by SEQ ID NO (DNA): 1714 and SEQ ID NO (PRT): 4108. Another exemplary trispecific double chain construct targeting CD19, CD20 and BCMA is represented by SEQ ID NO (DNA): 1619 and SEQ ID NO (PRT): 4013.

In some embodiments, the SAR of the disclosure comprises one, typically more than one vH (VH) domain, i.e., one or more vH single domain antibody, and is devoid of light chains. In an embodiment, the SAR comprises at least two vH single domain (SVH) antibodies.

In some embodiments, the SAR of the disclosure comprises one, typically more than one VHH domain, i.e., one or more VHH single domain antibody, and is devoid of light chains. In an embodiment, the SAR comprises at least two VHH single domains.

In some embodiments, the SAR of the disclosure comprises one, typically more than one non-immunoglobulin antigen binding scaffold, i.e. one or more domains selected from a DARPIN, an affibody, a ZIP domain (e.g., RZIP, EZIP, E4, R4 etc.), an affilin, an adnectin, an affitin, an obodies, a repebody, a fynomer, an alphabody, an avimer, an atrimer, a centyrin, a pronectin, an anticalin, a kunitz domain, an Armadillo repeat protein or a fragment thereof. In an embodiment, the SAR comprises two AABD. In an embodiment, the SAR comprises a Fv (i.e., vL/vH fragment that combine to form a Fv) and at least one AABD.

In some embodiments, a SAR of the disclosure comprises at least two AABD (e.g., two SVH domains or two VHH domains or one SVH and one VHH domain etc.) which target one or more antigens.

In some embodiments, the SAR of the disclosure comprises at least two antigen binding domains which target one or more antigens.

In one embodiment, the antigen binding domains of a SAR of the disclosure comprises two or at least two AABD (e.g., SVH, VHH, Centyrin etc.) that are specific for the same antigen, thus providing a bivalent binding molecule. In one embodiment, the antigen binding domain comprises two or at least two AABD (e.g., SVH, VHH, Centyrin etc.) that are specific for the same antigen but bind to different epitopes on said antigen. In other words, the antigen binding domain comprises a first AABD (e.g., SVH, VHH, Centyrin etc.) that binds to a first epitope and a second AABD (e.g., SVH, VHH, Centyrin etc.) that binds to a second epitope. The epitopes may be overlapping. Thus, the antigen binding domain is biparatopic and the scope of the disclosure includes a biparatopic SAR. In yet another embodiment, the antigen binding domain comprises two AABD (e.g., SVH, VHH, Centyrin etc.) that are specific for the same antigen and bind to the same epitopes on said antigen.

In one embodiment, the antigen binding domains of a SAR of the disclosure comprises a Fv (e.g., a vL fragment and a vH fragment that are attached to different signaling chains and are not present in a single chain fragment variable format or scFv format) and at least one AABD (e.g., SVH, VHH, Centyrin etc.) that is specific for the same antigen as bound by the Fv, thus providing a bivalent binding molecule. In one embodiment, the antigen binding domain comprises a Fv and at least one AABD (e.g., SVH, VHH, Centyrin etc.) that is specific for the same antigen as the Fv but bind to different epitopes on said antigen. In other words, the antigen binding domain comprises a Fv that binds to a first epitope and a second AABD (e.g., SVH, VHH, Centyrin etc.) that binds to a second epitope. The epitopes may be overlapping. Thus, the antigen binding domain is biparatopic and the scope of the disclosure includes a biparatopic SAR. In yet another embodiment, the antigen binding domain comprises a Fv and at least one AABD (e.g., SVH, VHH, Centyrin etc.) that are specific for the same antigen and bind to the same epitopes on said antigen. In yet another embodiment, the antigen binding domain comprises a Fv and at least one AABD (e.g., SVH, VHH, Centyrin etc.) where the Fv fragment does not bind to any specific antigen with significant affinity or binds with insignificant affinity and merely serves as a scaffold for the attachment of the one or more AABD.

In one embodiment, the antigen binding domains of a SAR of the disclosure comprises a TCR-Fv (e.g., a Vα/Vβ fragment or Vγ/Vδ fragments that are attached to different signaling chains and are not present in a single chain TCR format or scTCR format) and at least one AABD (e.g., SVH, VHH, Centyrin etc.) that is specific for the same antigen as bound by the TCR-Fv, thus providing a bivalent binding molecule. In one embodiment, the antigen binding domain comprises a TCR-Fv (e.g., Va/Vb or Vg/Vd) and at least one AABD (e.g., SVH, VHH, Centyrin etc.) that is specific for the same antigen as the TCR-Fv but bind to different epitopes on said antigen. In other words, the antigen binding domain comprises a TCR-Fv that binds to a first epitope and a second AABD (e.g., SVH, VHH, Centyrin etc.) that binds to a second epitope. The epitopes may be overlapping. Thus, the antigen binding domain is biparatopic and the scope of the disclosure includes a biparatopic SAR. In yet another embodiment, the antigen binding domain comprises a TCR-Fv and at least one AABD (e.g., SVH, VHH, Centyrin etc.) that are specific for the same antigen and bind to the same epitopes on said antigen. In yet another embodiment, the antigen binding domain comprises a TCR-Fv and at least one AABD (e.g., SVH, VHH, Centyrin etc.) where the TCR-Fv fragment does not bind to any specific antigen with significant affinity or binds with insignificant affinity and merely serves as a scaffold for the attachment of the one or more AABD.

In another embodiment, the antigen binding domain comprises two AABD (e.g., SVH, VHH, Centyrin etc.) that are specific for two different antigens, thus providing a bispecific antigen binding domain. In other words, the antigen binding domain comprises a first AABD (e.g., SVH, VHH, Centyrin etc.) that binds to a first target and a second AABD (e.g., SVH, VHH, Centyrin etc.) that binds to a second target. Thus, in certain embodiments, the disclosure relates to bispecific SARs.

As used herein, the term “bispecific SAR” or “bispecific antigen binding domain” thus refers to a polypeptide that comprises a binding molecule as described herein which has a binding site that has binding specificity for a first target antigen, and a second polypeptide domain which has a binding site that has binding specificity for a second antigen target, i.e., the bispecific binding molecule has specificity for two targets. The first target and the second target are not the same, i.e., are different targets, e.g., proteins; both may be present on a cell surface. Accordingly, a bispecific binding molecule as described herein can selectively and specifically bind to a cell that expresses (or displays on its cell surface) the first target and the second target. In another embodiment, the binding molecule comprises more than two antigen-binding domains providing a multispecific binding molecule. A multispecific antigen-binding domain as described herein can in addition to binding a first target bind one or more additional targets, i.e., a multispecific polypeptide can bind at least two, at least three, at least four, at least five, at least six, or more targets, wherein the multispecific polypeptide agent has at least two, at least, at least three, at least four, at least five, at least six, or more target binding sites respectively.

Antigen binding domains that comprise three or more AABD (e.g., SVH, VHH, Centyrin etc.) are therefore also within the scope of the disclosure.

In one aspect, the disclosure describes the optimal configuration of a SAR of the disclosure for targeting two or more antigens. In one aspect, the disclosure describes the optimal configuration of a SAR of the disclosure for targeting two or more epitopes of one or more antigens.

The single chain and double chain SARs of the disclosure can be expressed in T cells in which the expression of the endogenous TCRα, TCRβ, TCR and/or TCRS genes has been reduced or eliminated using methods known in the art. Such T cells in which the expression of endogenous functional TRAC, TRBC, TRGC and/or TRDC chains has been reduced or eliminated can be used for the purpose of allogeneic cell therapy.

In an embodiment, the single chain and double chain SARs of the disclosure can be targeted to the TRAC, TRBC, TRGC and/or TRDC locus in T cells using methods as described in PCT/US2018/053247 which is incorporated in its entirety by reference herein. Such T cells in which the endogenous TRAC, TRBC, TRGC and/or TRDC loci are disrupted by insertion of SAR can be used for the purpose of allogeneic cell therapy.

The disclosure also provides compositions and methods for targeting bispecific and multispecific SARs to the TRAC and/or TRBC loci for the purpose of generating allogeneic SAR-T cells.

In an embodiment, the single-chain and double chain SARs of the disclosure can be targeted to the endogenous loci encoding one or more genes that are expressed in immune cells, e.g., T cells, NK cells, NKT cells, monocytes, macrophages and/or neutrophils etc.

In an embodiment, the single-chain and double chain SARs of the disclosure can be targeted to the endogenous loci encoding one or more genes that are expressed in NK cells.

In an embodiment, the single-chain and double chain SARs of the disclosure can be targeted to the endogenous loci encoding one or more genes selected from the group of CD16A, CD16B, NKp30, NKp44, NKp46, KIR2DS4, DAP10, DAP12, FcRγ, CD3z, NKG2D, NKG2A and DNAM1.

In an embodiment the single-chain and double chain SARs of the disclosure are targeted to the endogenous loci encoding one or more of the CD16A, CD16B NKp30, NKp44, NKp46, KIR2DS4, DAP10, DAP12, FcRγ, CD3z, NKG2D, NKG2C and DNAM1 genes so that the antigen binding domain(s) of the SARs are expressed in frame with the partial or entire extracellular domain, hinge domain, and/or transmembrane domains of the CD16A, CD16B, NKp30, NKp44, NKp46, DAP10, DAP12, FcRγ, CD3z, NKG2D, NKG2C and DNAM1 genes.

In an embodiment the single-chain and double chain SARs of the disclosure are targeted to the endogenous loci encoding one or more of the CD16A, CD16B, NKp30, NKp44, NKp46, KIR2DS4, DAP10, DAP12, FcRγ, CD3z, and DNAM1 genes so that the antigen binding domain(s) of the SARs are inserted downstream of and in-frame with the signal peptides encoding the CD16A, CD16B, NKp30, NKp44, NKp46, KIR2DS4, DAP10, DAP12, FcRγ, CD3z, and DNAM1 genes.

Methods for generation of immune cells, including T and NK cells, by directed differentiation of genomic engineered iPSC are known in the art, including in WO2020117526, WO2020210398, WO2019126748, WO2019112899, WO2019018603 and U.S. Ser. No. 10/370,452, which are incorporated in their entirety by reference herein.

In one aspect the novel antigen binding domain of a SAR binds to an antigen preferentially or exclusively expressed on hematopoietic lineage cells. Exemplary antigens that are preferentially or exclusively expressed on hematopoietic lineage cells are CD19, CD20, CD22, BCMA, CS1, CD33, MPL, CD138, CD38 and CD123. In one aspect the novel antigen binding domain of a SAR binds to an antigen preferentially or exclusively expressed on non-hematopoietic lineage cells. Exemplary antigens that are preferentially or exclusively expressed on non-hematopoietic lineage cells are Mesothelin (MSLN), Her2, EGFR, PSMA, PSCA, GPC3 and the like. In one aspect the SAR expresses two or more novel antigen binding domains where at least one of the novel antigen binding domain binds to an antigen preferentially or exclusively expressed on hematopoietic lineage cells and at least one of the novel antigen domain binds to an antigen expressed on non-hematopoietic lineage cells.

The disclosure provides that two or more AABD of a SAR (e.g., SVH, VHH, Centyrin etc.) may be connected by a linker, for example a peptide linker. A linker may be also present between the vL and/or vH domain comprising the Fv or TCR-Fv and the AABD. Suitable linkers, for example comprising linker include GS residues such as (Gly₄Ser)ₙ, where n=from 1 to 10, e.g., 1, 2, 3, 4, 5, 6, 7, 8, 9 or 10. Exemplary linkers are provided in SEQ ID NO (DNA): 1024-1028 and SEQ ID NO (PRT): 3418-3422.

A linker may be also present between the vL and vH domain comprising the Fv or TCR-Fv of a SAR and the TCR constant chain connecting peptide to which the vL and vH domains are attached. In particular, the IgCL (SEQ ID NO (DNA): 1142 and SEQ ID NO (PRT): 3536) and IgCH domains (SEQ ID NO (DNA): 1143-1157 and SEQ ID NO (PRT): 3537-3551) derived from antibodies serve as useful linkers between the vL/vH and signaling chains. Additional Ig like domains are known in the art (e.g., SEQ ID NO (DNA):1168-1175 and SEQ ID NO (PRT):3562-3569) and can serve as useful linkers in alternate embodiment of the disclosure.

In some embodiments, the one or more AABD comprising the antigen binding domain of the SAR are attached to the signaling chain without the intervening vL/vH fragments. In such constructs, a linker may be also present between the AABD of the SAR and the signaling chain to which the AABD are attached. In an embodiment, one of the AABD of a double chain SAR is attached to the linker IgCL (SEQ ID NO: 1142) and the other AABD is attached to the linker IgG1-CH1 (SEQ ID NO: 1143). In an embodiment, one of the AABD of a double chain SAR is attached to the linker IgCL (SEQ ID NO: 1142) and the other AABD is attached to the linker IgG4-CHI1 (SEQ ID NO: 1152). In an embodiment, one of the AABD of a double chain SAR is attached to the linker TCRa-wt-opt-6ECD (SEQ ID NO: 1158) and the other AABD is attached to the linker TCRb-wt-opt-6ECD (SEQ ID NO: 1160).

In some embodiments, the antigen binding domain of a SAR polypeptide molecule is derived from or comprises of vL and vH domains of an antibody that are separately attached to the NH2-terminus or near the NH2-terminus of two signaling chains (e.g., CD16A, NKp30, NKp44, NKp46, DAP10, DAP12 etc. or mutants or variant thereof as described herein) to jointly constitute a Fragment variable (Fv) that binds to a specific antigen.

In some embodiments, the antigen binding domain of a SAR polypeptide molecule is derived from or comprises of Va and Vβ domains of a TCR that are separately attached to the NH2-terminus or near the NH2-terminus of two signaling chains (e.g., CD16A, NKp30, NKp44, NKp46, CD3z, DAP10, DAP12 etc. or mutants or variant thereof as described herein) to jointly constitute a Fragment variable-TCR (TCR-Fv) that binds to a specific peptide antigen in association with an MHC molecule.

In some embodiments, the antigen binding domain of a SAR polypeptide molecule is derived from or comprises of V_{y} and Vδ domains of a TCR that are separately attached to the NH2-terminus or near the NH2-terminus of two polypeptide chains (e.g., CD16A, NKp30, NKp44, NKp46, CD3z, DAP10, DAP12 etc. or mutants or variant thereof as described herein) to jointly constitute a Fragment variable-TCR (TCR-Fv) that binds to a specific peptide antigen/MHC or a lipid antigen.

In some embodiments, the SAR polypeptide has an antigen binding domain that is expressed as single chain variable fragments (scFv) and is operationally linked to the NH2-terminus or near the NH2-terminus of a signaling chain (e.g., CD16A, NKp30, NKp44, NKp46, DAP10, DAP12 etc. or mutants or variant thereof as described herein).

In certain embodiments, the AABDs of the two polypeptide chains of a double chain SAR are similar in structure (e.g., both AABD are SVH or camelid VHH domain or affibodies or Centyrins). In one embodiment, the AABD of the two polypeptide chains of a double chain SAR are not similar in structure (e.g., the first antigen binding domain is a SVH and the second antigen binding domain is a camelid VHH).

In some embodiments, the antigen binding domain of the encoded SAR polypeptides is encoded by a codon optimized nucleotide sequence of the corresponding wild-type sequence or a non-wild-type sequence antibody, single domain antibodies (SDAB), VH domains, VL domain, camelid VHH domains, or a non-immunoglobulin scaffolds such as DARPINs, affibodies, affilins, adnectins, affitins, obodies, repebodies, fynomers, alphabodies, avimers, atrimers, centyrins, pronectins, anticalins, kunitz domains, Armadillo repeat proteins, autoantigen, receptors or ligands.

In some embodiments, the encoded one or more antigen binding domains of the SAR polypeptide comprise any one or more of light chain variable domain (vL or VL) amino acid sequences of SEQ ID NO 2440 to 2676 wherein up to 20 amino acid residues but no more than 21 amino acids are replaced by any other amino acid residues, or sequences with 70-99.9% identity to amino acid sequences of SEQ ID NO 2440 to 2676, or sequences with 70-100% identity to the complementarity determining regions (CDR's) of SEQ ID NO: 2440 to 2676, or sequences with up to 3 amino acid substitution in each of the three complementarity determining regions of 10736 to 10972. Table 3 shows the target antigens, names, SEQ ID NO (DNA), SEQ ID NO (PRT), SEQ ID NO (PRT) of scFv of the exemplary vL domains used in this disclosure.

In some embodiments, the encoded one or more antigen binding domains of the SAR polypeptide comprise any one or more of heavy chain variable domain (vH or VH) amino acid sequences of SEQ ID NO: 2682-2918 wherein up to 20 amino acid residues but no more than 21 amino acids are replaced by any other amino acid residues, or sequences with 70-99.9% identity to amino acid sequences of SEQ ID NO 2682-2918. In some embodiments, the encoded one or more antigen binding domains of the SAR polypeptide comprise any one or more of heavy chain variable domain (vH or VH) amino acid sequences of SEQ ID NO: 2682-2918 wherein up to 20 amino acid residues but no more than 21 amino acids are replaced by any other amino acid residues, or sequences with 70-99.9% identity to amino acid sequences of SEQ ID NO: 2682-2918, or sequences with 70-100% identity to the complementarity determining regions (CDR's) of SEQ ID NO: 2682-2918, or sequences with up to 3 amino acid substitution in any of the three complementarity determining regions of SEQ ID NO: 2682-2918. Table 3 shows the target antigens, names, SEQ ID NO (DNA), SEQ ID NO (PRT), SEQ ID NO (PRT) of scFv of the exemplary vH domains used in this disclosure.

In some embodiments, the encoded one or more antigen binding domains of the SAR polypeptide comprise any one or more of camelid single domain antibody (vHH or VHH) amino acid sequences of SEQ ID NO:3253-3296 wherein up to 20 amino acid residues but no more than 21 amino acids are replaced by any other amino acid residues, or sequences with 70-99.9% identity to amino acid sequences of SEQ ID NO 3253-3296, or sequences with up to 3 amino acid substitution in any of the three complementarity determining regions (CDR's) of SEQ 3253-3296.

In some embodiments, the encoded one or more antigen binding domains of the SAR polypeptide comprise any one or more of non-immunoglobulin antigen binding scaffold amino acid sequences of SEQ ID NO: 3366-3377 wherein up to 20 amino acid residues but no more than 21 amino acids are replaced by any other amino acid residues, or sequences with 70-99% identity to amino acid sequences of SEQ ID NO: 3366-3377.

In some embodiments, the encoded one or more antigen binding domains of the SAR polypeptide comprise any one or more of receptor amino acid sequences of SEQ ID NO 3378-3395 wherein up to 20 amino acid residues but no more than 21 amino acids are replaced by any other amino acid residues, or sequences with 70-99.9% identity to amino acid sequences of SEQ ID NO: 3378-3395.

In some embodiments, the encoded one or more antigen binding domains of the SAR polypeptide comprise autoantigen amino acid sequences of SEQ ID NO:3391 wherein up to 19 amino acid residues but no more than 20 amino acids are replaced by any other amino acid residues, or sequences with 70-100% identity to amino acid sequences of SEQ ID NO 3391.

In some embodiments, the encoded one or more antigen binding domains of the SAR molecule comprise any one or more of ligand amino acid sequences of SEQ ID NO: 3396-3406 wherein up to 20 amino acid residues but no more than 21 amino acids are replaced by any other amino acid residues or sequences with 70-100% identity to amino acid sequences of SEQ ID NO: 3396-3406.

In some embodiments, the encoded one or more antigen binding domains of the SAR polypeptide comprise any one or more of scFv amino acid sequences of SEQ ID NO: 2924-3160 wherein up to 40 amino acid residues but no more than 41 amino acids are replaced by any other amino acid residues, or sequences with 70-100% identity to amino acid sequences of SEQ ID NO 2924-3160 or sequences with 70-100% identity in the six complementarity determining regions (CDR's) in each of SEQ ID NO 2924-3160 or sequences with up to 3 substitution in any of the six complementarity determining regions (CDR's) in each of SEQ ID NO: 2924-3160.

In some embodiments, the encoded one or more antigen binding domains of the SAR polypeptide comprise any one or more of an antigen binding portions, e.g., CDRs, of vL and vH fragments targeting this antigen or domains with up to 3 amino acid substitutions in any of the CDRs of the vL and vH fragments listed in Table 3. The sequences of the CDR1-3 of the vL and vH fragments listed in Table 3 can be determined by methods known in the art.

In some embodiments, the encoded one or more antigen binding domains of the SAR polypeptide comprise any one or more of an antigen binding portions, e.g., CDRs, of vHH fragments targeting this antigen.

In one embodiment, an antigen binding domain of a SAR is an antigen binding portion of a receptor known to bind this target antigen.

In another embodiment, the disclosure provides SARs that bind to the same epitope on the different targets as any of the SARs of the disclosure (i.e., SARs that have the ability to cross-compete for binding to the different targets with any of the SARs of the disclosure). In some embodiments, the antigen specific domains of these SARs could be determined from vL fragments, vH fragments and/or scFv fragments of the antibodies that were used as the component of the SAR. In some embodiments, the reference antibodies for cross-competition studies to determine the target-epitope recognized by a SAR of the disclosure are vL, vH, scFvs, SVH, vHH, non-immunoglobulin antigen binding domains described herein. In an exemplary embodiment, the reference scFv hu-mROO5-1 represented by SEQ ID NO:3027 can be used in cross-competition studies to determine the target-epitope recognized by hu-mROO5-1-based SARs of the disclosure. In some embodiments, the reference AABD fragments for cross-competition studies to determine the target-epitope recognized by a SAR of the disclosure described are AABD fragments described here. In some embodiments, the reference non-immunoglobulin antigen binding scaffolds for cross-competition studies for cross-competition studies to determine the target-epitope recognized by a SAR of the disclosure described are non-immunoglobulin antigen binding scaffolds-based AABD. In some embodiments, the reference ligands for cross-competition studies to determine the target-epitope recognized by a SAR of the disclosure are ligands.

In other embodiments described herein, the bispecific SAR of the disclosure shows more than 30% (e.g., more than 40%, 50%, 60%, 70%, 80%, 90%, or 95%, 99% etc.) affinity for each of the target antigens as compared to the affinity of the each of the corresponding unispecific SARs when expressed in an effector cell and compared under similar conditions. The binding affinity can be measured using assays known in the art such as the Topanga Assay.

In other embodiments described herein, the bispecific SAR of the disclosure shows more than 30% (e.g., more than 40%, 50%, 60%, 70%, 80%, 90%, or 95%, 99% etc.) signaling activity against each of the target antigen expressing cell as compared to the signaling activity of each of the corresponding unispecific SARs when expressed in an effector cell and compared under similar conditions. The signaling activity can be measured using methods known in the art, such as the Jurkat-NFAT-GFP cell assay.

In other embodiments described herein, the bispecific SAR of the disclosure shows more than 30% (e.g., more than 40%, 50%, 60%, 70%, 80%, 90%, or 95%, 99% etc.) cytokine (e.g., TNFα, IFNγ, IL-2 etc.) production against each of the target antigen expressing cell as compared to the cytokine production of each of the corresponding unispecific SARs when expressed in an effector cell and compared under similar conditions. The cytokine production (e.g., TNFα, IFNγ, IL-2 etc.) can be measured using methods known in the art, such as ELISA.

In other embodiments described herein, the bispecific SAR of the disclosure shows more than 30% (e.g., 40%, 50%, 60%, 70%, 80%, 90%, or 95%, 99% etc.) of the cytotoxic activity against each of the target antigen expressing cell as compared to the cytotoxic activity of each of the corresponding unispecific SARs when expressed in an effector cell and compared under similar conditions. The cytotoxic activity can be measured using methods known in the art, such as the Matador or radioactive chromium release assay.

In other embodiments described herein, the bispecific SAR of the disclosure shows more than 30% (e.g., 40%, 50%, 60%, 70%, 80%, 90%, or 95%, 99% etc.) of the in vivo activity against each of the target antigen expressing cell as compared to the in vivo activity of each of the corresponding unispecific SARs when expressed in an effector cell and compared under similar conditions. In an embodiment, the in vivo activity is measured using xenograft model in immunodeficient mice.

In other embodiments described herein, the multi-specific SAR of the disclosure shows more than 30% (e.g., more than 40%, 50%, 60%, 70%, 80%, 90%, or 95%, 99% etc.) affinity for each of the target antigens as compared to the affinity of the each of the corresponding unispecific SARs when expressed in an effector cell and compared under similar conditions. The binding affinity can be measured using assays known in the art such as the Topanga Assay.

In other embodiments described herein, the multi-specific SAR of the disclosure shows more than 30% (e.g., more than 40%, 50%, 60%, 70%, 80%, 90%, or 95%, 99% etc.) of the signaling activity against each of the target antigen expressing cell as compared to the signaling activity of each of the corresponding unispecific SARs when expressed in an effector cell and compared under similar conditions. The signaling activity can be measured using methods known in the art, such as the Jurkat-NFAT-GFP cell assay.

In other embodiments described herein, the multi-specific SAR of the disclosure shows more than 30% (e.g., 40%, 50%, 60%, 70%, 80%, 90%, or 95%, 99% etc.) of the cytotoxic activity against each of the target antigen expressing cell as compared to the cytotoxic activity of each of the corresponding unispecific SARs when expressed in an effector cell and compared under similar conditions. The cytotoxic activity can be measured using methods known in the art, such as the Matador or radioactive chromium release assay.

In other embodiments described herein, the multispecific SAR of the disclosure shows more than 30% (e.g., more than 40%, 50%, 60%, 70%, 80%, 90%, or 95%, 99% etc.) cytokine (e.g., TNFα, IFNγ, IL-2 etc.) production against each of the target antigen expressing cell as compared to the cytokine production of each of the corresponding unispecific SARs when expressed in an effector cell and compared under similar conditions. The cytokine production (e.g., TNFα, IFNγ, IL-2 etc.) can be measured using methods known in the art, such as ELISA.

In other embodiments described herein, the multi-specific SAR of the disclosure shows more than 30% (e.g., 40%, 50%, 60%, 70%, 80%, 90%, or 95%, 99% etc.) of the in vivo activity against each of the target antigen expressing cell as compared to the in vivo activity of each of the corresponding unispecific SARs when expressed in an effector cell and compared under similar conditions. In an embodiment, the in vivo activity is measured using xenograft model in immunodeficient mice.

In some embodiments, when present on the surface of a cell, binding affinity of the antigen binding domain comprised by the Fv or TCR-Fv (i.e., vL/vH, Va/V or Vy/VS fragments) of a bispecific SAR to its cognate antigen is not substantially reduced by one or more AABDs that are attached to the N-terminus region of the vL, vH, Va, VD, Vγ or V6 fragment of the said SAR. In an embodiment, the SAR is a single chain SAR. In an embodiment, the SAR is a double chain SAR.

In some embodiments, when present on the surface of a cell, the antigen binding affinity of the antigen binding domain comprised by the Fv or TCR-Fv (i.e., vL/vH, Vα/Vβ or Vγ/Vδ fragments) of a bispecific SAR to its cognate antigen comprising one or more AABDs that are attached to the N-terminus region or near the N-terminus of the vL, vH, Vα, Vβ, Vγ and/or Vδ fragments of the said SAR is at least 70%, 80%, 85%, 90%, 95%, 96%, 97%, 98% or 99% of antigen binding affinity of the antigen binding domain of a corresponding unispecific SAR in which one or more AABDs are not attached to the N-terminus region or near the N-terminus region of the vL, vH, Vα, Vβ, Vγ or Vδ fragments.

In some embodiments, binding of the antigen binding domain of said first chain of SAR to its cognate antigen in the presence of said second chain of SAR is 70%, 80%, 85%, 90%, 95%, 96%, 97%, 98% or 99% of binding of the antigen binding domain of said first chain of SAR to its cognate antigen in the absence of said second chain of SAR to its cognate antigen.

In some embodiments, binding of the antigen binding domain of said first chain of SAR to its cognate antigen in the presence of said second chain of SAR (or a CAR) is 70%, 80%, 85%, 90%, 95%, 96%, 97%, 98% or 99% of binding of the antigen binding domain of said first chain of SAR to its cognate antigen in the absence of said second chain of SAR (or a CAR) to its cognate antigen.

In one embodiment, the VH domain is selected from SEQ ID NO: 3210-3252 having one or more amino acid substitutions, deletions, insertions or other modifications compared to SEQ ID NOs: 3210-3252, and which retains a biological function of the single domain antibody.

In another embodiment, the VH domain is selected from one of the SEQ ID NOs. 3210-3252, but comprises one or more amino acid substitutions, for example 1 to 20, such as 1, 2, 3, 4, 5, 6, 7, 8, 9 or 10 amino acid substitutions. In one embodiment, the one or more amino acid substitution is in one or more of the framework areas. In another embodiment, the one or more amino acid substitution is in one or more of the CDRs. In one embodiment, the amino acid substitutions are in the framework and CDR sequences.

The disclosure also comprises sequence optimized variants of the single domain antibodies described herein.

In one embodiment, the binding domain of the SAR of the disclosure provides biparatopic targeting to BCMA. Thus, the binding domain comprises a first AABD (e.g., VH single domain antibody) that binds to a first epitope of BCMA and an AABD (e.g., VH single domain antibody) that binds to a second epitope of BCMA. The first and second epitope may be overlapping.

In one embodiment, the binding domain of the SAR of the disclosure provides biparatopic targeting to CD22. Thus, the binding domain comprises a first VH single domain antibody that binds to a first epitope of CD22 and a second VH single domain antibody that binds to a second epitope of CD22. The first and second epitope may be overlapping.

In one embodiment, the binding domain of the SAR of the disclosure provides biparatopic targeting to CD38. Thus, the binding domain comprises a first VH single domain antibody that binds to a first epitope of CD38 and a second VH single domain antibody that binds to a second epitope of CD38. The first and second epitope may be overlapping.

In one embodiment, the binding domain of the SAR of the disclosure provides biparatopic targeting to CEA. Thus, the binding domain comprises a first VH single domain antibody that binds to a first epitope of CEA and a second VH single domain antibody that binds to a second epitope of CEA. The first and second epitope may be overlapping.

In one embodiment, the binding domain of the SAR of the disclosure provides biparatopic targeting to PSMA. Thus, the binding domain comprises a first VH single domain antibody that binds to a first epitope of PSMA and a second VH single domain antibody that binds to a second epitope of PSMA. The first and second epitope may be overlapping.

In one embodiment, the binding domain of the SAR of the disclosure provides bispecific targeting. Thus, the binding domain comprises a VH single domain antibody that binds BCMA and a second binding moiety that targets a second target. The second binding moiety may be an antibody fragment, typically a VH single domain antibody, a centyrin, an affibody or a vHH domain. The second target may be selected from CD19, CD20, CD22, BCMA, PSCA, CS1, GPC3, CSPG4, EGFR, 5T4, L1 CAM, MUC16, ROR1, cKit, ROR1, mesothelin, IL3Ra, c-Met, EGFRvlll, GD-2, NY-ESO-1 TCR or MAGE A3 TCR, HER2, Wilm's tumor gene 1 (WT1), carcinoembryonic antigen (CEA), mucin 16, MUC1, an immuno checkpoint target or combinations thereof. However, a skilled person would understand that other tumor antigens are also potential combination targets within the scope of the disclosure. In the case of a single chain SAR, the two binding domains may be present in either order.

In one embodiment, the first binding domain of SAR comprises an AABD (e.g., VH single domain antibody or SVH, vHH or Centyrin etc.) that binds BCMA and a second binding moiety that targets CD38. In an embodiment, the SAR further comprises vL/vH fragments that combine to form a Fv targeting a specific antigen. Exemplary vL/vH fragments and their target antigens are provided in Table 3. In an embodiment, the SAR further comprises Va/Vb or Vg/Vd fragments that combine to form a TCR-Fv targeting different antigens. Exemplary Va/Vb or Vg/Vd fragments and their target antigens are provided in Table 4.

In one embodiment, one binding domain of SAR comprises an AABD (e.g., VH single domain antibody or SVH, vHH or Centyrin etc.) that binds BCMA and a second binding moiety that targets CD19. In an embodiment, the SAR further comprises vL/vH fragments that combine to form a Fv targeting a specific antigen. Exemplary vL/vH fragments and their target antigens are provided in Table 3. In an embodiment, the SAR further comprises Va/Vb or Vg/Vd fragments that combine to form a TCR-Fv targeting different antigens. Exemplary Va/Vb or Vg/Vd fragments and their target antigens are provided in Table 4.

In one embodiment, the first binding domain of SAR comprises an AABD (e.g., VH single domain antibody or SVH, vHH or Centyrin etc.) that binds BCMA and a second binding moiety that targets CD22. In an embodiment, the SAR further comprises vL/vH fragments that combine to form a Fv targeting a specific antigen. Exemplary vL/vH fragments and their target antigens are provided in Table 3. In an embodiment, the SAR further comprises Va/Vb or Vg/Vd fragments that combine to form a TCR-Fv (or TCR-Fv) targeting different antigens. Exemplary Va/Vb or Vg/Vd fragments and their target antigens are provided in Table 4.

In one embodiment, the first binding domain of SAR comprises an AABD (e.g., VH single domain antibody or SVH, vHH or Centyrin etc.) that binds BCMA and a second binding moiety that targets CD20. In an embodiment, the SAR further comprises vL/vH fragments that combine to form a Fv targeting a specific antigen.

In one embodiment, the first binding domain of SAR comprises an AABD that binds CD22 and a second binding moiety that targets CD20.

In one embodiment, the first binding domain of SAR comprises an AABD that binds CD22 and a second binding moiety that targets CD19. In an embodiment, the SAR further comprises vL/vH fragments that combine to form a Fv targeting a specific antigen. In an embodiment, the SAR further comprises Va/Nb or Vg/Vd fragments that combine to form a TCR-Fv targeting different antigens.

In one embodiment, the first binding domain of SAR comprises an AABD that binds CD19 and a second binding moiety that targets CD20. In an embodiment, the SAR further comprises vL/vH fragments that combine to form a Fv targeting a specific antigen. In an embodiment, the SAR further comprises Va/Nb or Vg/Vd fragments that combine to form a TCR-Fv targeting different antigens.

In one embodiment, the first binding domain of SAR comprises an AABD that binds CD19 and a second binding moiety that targets CD38. In an embodiment, the SAR further comprises vL/vH fragments that combine to form a Fv targeting a specific antigen. In an embodiment, the SAR further comprises Va/Nb or Vg/Vd fragments that combine to form a TCR-Fv targeting different antigens.

In one embodiment, the first binding domain of SAR comprises AABD that binds CD19 and a second binding moiety that targets CD123. In an embodiment, the SAR further comprises vL/vH fragments that combine to form a Fv targeting a specific antigen. In an embodiment, the SAR further comprises Va/Nb or Vg/Vd fragments that combine to form a TCR-Fv targeting different antigens.

In one embodiment, the first binding domain of SAR comprises AABD that binds CD19 and a second binding moiety that targets BAFF-R. In an embodiment, the SAR further comprises vL/vH fragments that combine to form a Fv targeting a specific antigen. In an embodiment, the SAR further comprises Va/Vb or Vg/Vd fragments that combine to form a TCR-Fv targeting different antigens.

In addition to a binding domain as described in detail above, the SAR of the disclosure may further comprise one or more signaling chains or fragments and variants thereof. Exemplary signaling chains and fragments are provided in SEQ ID NO (DNA):1349-1584 and 8541 to 8767 (new chains) and SEQ NO (PRT):3743-3966, 3385, 3394, 7818-7822, 9633-9859. The disclosure also provides exemplary components of signaling chains such as extracellular domains, extracellular and transmembrane domains, transmembrane domains, hinge domains (SEQ ID NO (DNA):1535-1549 and SEQ ID NO (PRT):3929-3943), cytosolic domains (SEQ ID NO (DNA):1550-1564 and SEQ ID NO (PRT):3944-3958) and costimulatory domains (SEQ ID NO (DNA):1565-1572 and SEQ ID NO (PRT):3959-3966). In an exemplary embodiment, the signaling chains and fragments for the construction of the SARs comprise of polypeptides represented by SEQ ID NOs: 3743-3966 3385, 3394, 7818-7822, 9633-9859 or fragments with at least 60%, 70%, 80% or 90% homology thereto or functional variants thereof or the equivalent residues (i.e., a homolog) from a non-human species, e.g., mouse, rodent, monkey, ape and the like. It is to be understood that signaling chains and fragments from other mammalian species can be used in the methods of disclosure to make SARs of the disclosure. Furthermore, signaling chains and fragments that are hybrids of signaling chains and fragments derived from human and other mammalian species can be used in the methods of disclosure to make SARs of the disclosure. Finally, alternatively spliced isoforms of the signaling chains and fragments described in can be used in the methods of disclosure to make SARs.

The SARs of the disclosure comprise one or more transmembrane domains. A “transmembrane domain” (TMD) as used herein refers to the region of the SAR which crosses the plasma membrane and is connected to the endoplasmic signaling domain and the antigen binding domain, in case of the latter optionally via a hinge domain or a connecting peptide. In one embodiment, the transmembrane domain of the SAR of the disclosure is the transmembrane region of a Type I or a Type II transmembrane proteins, or an artificial hydrophobic sequence or a combination thereof. In one embodiment, the transmembrane domain comprises the transmembrane domain derived from CD16, CD64, CD32, KIR2DS4, CD3z, NKp30, NKp44, NKp46, NKG2D, DAP10, DAP12, CD28 and CD8. Other transmembrane domains will be apparent to those of skill in the art and may be used in connection with alternate embodiments of the disclosure. Specifically, within the scope of disclosure are TMD sequences shown in SEQ ID NO: 3914-3928.

The SAR of the disclosure further comprises an intracellular signaling domain. An “intracellular signaling domain”, “cytoplasmic domain” or “endodomain” is the domain that transmits activation signals to T cells and directs the cell to perform its specialized function. Specifically within the scope of disclosure is intracellular signaling domain sequences shown in SEQ ID NO: 3944-3958.

In one embodiment, the SAR of the disclosure further comprises one or more co-stimulatory domains to enhance SAR-T cell activity after antigen specific engagement. Multiple co-stimulatory domains can be included in a single SAR to recruit multiple signaling pathways. Specifically within the scope of disclosure are co-stimulatory domains sequences with SEQ ID NO: 3959-3966.

In one embodiment, the SAR of the disclosure further comprises a hinge or spacer region which connects the extracellular antigen binding domain and the transmembrane domain. This hinge or spacer region can be used to achieve different lengths and flexibility of the resulting SAR. Examples of a hinge or spacer region that can be used according to the disclosure include, but are not limited to, Fc fragments of antibodies or fragments or derivatives thereof, hinge regions of antibodies, or fragments or derivatives thereof, C_{H}2 regions of antibodies, C_{H}3 regions of antibodies, CD8A hinge domain, CD28 hinge domain, CD16 hinge domain, NKp30 hinge domain, NKp44 hinge domain, NKp46 hinge domain and artificial spacer sequences, for example peptide sequences, or combinations thereof. Other hinge or spacer region will be apparent to those of skill in the art and may be used in connection with alternate embodiments of the disclosure. Specifically, within the scope of disclosure are hinge sequences shown in SEQ ID NO: 3592-3598 and 3929-3943. The TCR connecting peptides (SEQ ID NO: 3571-3579) can also serve as the hinge domains.

In one embodiment, the SAR of the disclosure further comprises a “linker domain” or “linker region” that connects different domains of the SAR. This domain includes an oligo- or polypeptide region from about 1 to 500 amino acids in length. Suitable linkers will be apparent to those of skill in the art and may be used in connection with alternate embodiments of the disclosure.

In one embodiment, the SAR of the disclosure further comprises a “leader sequence”. In one embodiment, the leader sequence is a CD8A domain. Specifically, within the scope of disclosure are leader sequences with SEQ ID NO: 2425-2430.

In some aspects, the SAR of the disclosure includes an antigen binding domain that transmits an inhibitory signal.

In some aspects, the SAR of the disclosure includes an adaptor binding domain that allows it to bind to a soluble polypeptide adaptor or a tag. Exemplary adaptors and adaptor binding domains are provided in SEQ ID NO: 3407-3435. In an exemplary embodiment, a RZIP encoding SAR can be used in conjunction with an EZIP encoding polypeptide (SEQ ID NO: 3409) containing an antigen binding domain targeting CD19 to target a CD19-expressing cell. Similarly, a NKG2D-AF-G4Sx3-NKG2D-AF encoding SAR (e.g., SEQ ID NO: 5481) can be used in combination with a ULBP2R encoding polypeptide (e.g., CD8SP-BCMA-FHVH93-GS-ULBP2R, SEQ ID NO: 5131) to target BCMA. In an alternate embodiment, a NKG2D-YA-G4Sx3-NKG2D-YA encoding SAR (e.g., SEQ ID NO: 5482) can be used in combination with a ULBP2-S3 encoding polypeptide (e.g., CD8SP-BCMA-FHVH93-GS-ULBP2-S3, SEQ ID NO: 5132) to target BCMA. Similarly, a SAR encoding an antigen binding domain targeting Streptag (SEQ ID NO: 4970) or FITC (e.g., SEQ ID NO: 4963-4964) can be used in combination with a Streptag-labelled or FITC-labelled antibody/antibody fragment to target an antigen bound by the latter. Other adaptors are known in the art (e.g., WO2019099440 and Diana Darowski et al, MABS, 2019, VOL. 11, NO. 4, 621-631) and can be used in alternate embodiment of the disclosure.

The SAR may further include a label, for example a label that facilitates imaging, such as a fluorescent label or other tag. This can, for example be used in methods for imaging tumor binding. The label may be conjugated to the antigen binding domain.

The SARs described herein may be synthesized as single polypeptide chains. In this embodiment, the antigen-specific targeting regions are at the N-terminus, arranged in tandem and are separated by a linker peptide.

In another aspect, the disclosure provides an isolated SAR polypeptide molecule comprising one or more antigen binding domains (e.g., antibody or antibody fragment, a ligand or a receptor) that bind to antigens as described herein, and are jointed to one or more signaling chains.

In some embodiments, a SAR may comprise or consist of a single polypeptide that contains a single antigen binding domain joined to the NH2-terminus of a single signaling chain (Class 1).

In some embodiments, a SAR comprises or consists of two polypeptides that assemble to make a functional SAR (Class 2). Each of the polypeptides of such dual chain Class 2 SAR contains a signaling chain and contains (as in Class 2A) or does not contain (as in Class 2B) one or more antigen binding domains. In Class 2A SARs, each of the antigen binding domains is joined to the N-terminus of a separate signaling chain. For example, antigen binding domain 1 (e.g., vL fragment of an antibody) is joined to one DAP10 chain to constitute functional polypeptide unit 1 and antigen binding domain 2 (vH fragment of an antibody) is joined to the second DAP10 chain to constitute functional polypeptide unit 2. The two functional polypeptide units of such SAR are coexpressed in the same cell and pair with each other to become functionally active. It should be noted that each of the antigen binding domains may in turn be bispecific or multispecific, thereby allowing the Class 2 SARs to target more than 2 antigens.

In some embodiments, the two functional polypeptide units of Class 2 SARs are coexpressed in a cell using different vectors. In some embodiments, the two functional polypeptide units of the Class 2 SARs are coexpressed in a cell using a single vector which employs two separate regulatory elements (e.g., promoters) to encode for two polynucleotides encoding the two functional polypeptide units of Class 2 SARs. The disclosure provides that small promoters that can be used to express the second polypeptide unit or a third accessory module is EFS promoter, EFS2 promoter or an RSV promoter. In some embodiments, the two functional polypeptide units of the Class 2 SARs are coexpressed in a cell using a single vector which employs a single promoter to express a polynucleotide containing an IRES sequence that separates the nucleotide fragments encoding the two polypeptides of the SAR. In some embodiments, the two functional polypeptide units of the Class 2 SARs are coexpressed in a cell using a single vector which employs a single promoter to express a polynucleotide encoding for a single polypeptide containing a cleavable linker (e.g., F2A, T2A, E2A, P2A etc.). The resulting mRNA encodes a single polypeptide which subsequently generates the two functional polypeptide units of the SAR. In some embodiments, the two functional polypeptide units of the Class 2 SARs are coexpressed using transfection of a single mRNA sequence that encodes for both functional polypeptide units, while in other embodiments the two functional polypeptide units are coexpressed by transfection of two different mRNA sequences, each encoding for one functional polypeptide unit. In some embodiments, the vector or mRNA encoding the SAR may encode for additional genes/proteins (therapeutic controls, inhibitory molecules, accessory modules etc.), which may be separated from the SAR encoding sequences by IRES or cleavable linkers expressed using separate promoters (e.g., EFS, EFS2 or RSV promoter) or combination thereof. In another embodiment, a therapeutic control or accessory module or both could be expressed in the cell in which SAR is expressed using a separate vector or mRNA. It is to be understood that therapeutic controls or accessory modules are not essential for the function of a SAR and any of the SAR of the embodiment can be used without therapeutic control or the accessory modules. For example, the antibiotic resistance cassette, such as PAC (puromycin resistance gene), can be removed from the SAR-encoding vectors of this disclosure without compromising the functionality of the SAR.

Also provided are functional variants of the SARs described herein, which have substantial or significant sequence identity or similarity to a parent SAR, which functional variant retains the biological activity of the SAR of which it is a variant. Functional variants encompass, for example, those variants of the SAR described herein (the parent SAR) that retain the ability to recognize target cells to a similar extent, the same extent, or to a higher extent, as the parent SAR. In reference to the parent SAR, the functional variant can, for instance, be at least about 30%, about 50%, about 75%, about 80%, about 85%, about 90%, about 91%, about 92%, about 93%, about 94%, about 95%, about 96%), about 97%, about 98%, about 99% or more identical in amino acid sequence to the parent SAR.

A functional variant can, for example, comprise the amino acid sequence of the parent SAR with at least one conservative amino acid substitution. Alternatively or additionally, the functional variants can comprise the amino acid sequence of the parent SAR with at least one non-conservative amino acid substitution. In this case, it is common for the non-conservative amino acid substitution to not interfere with or inhibit the biological activity of the functional variant. The non-conservative amino acid substitution may enhance the biological activity of the functional variant, such that the biological activity of the functional variant is increased as compared to the parent CAR.

The SARs (including functional portions and functional variants) can be of any length, i.e., can comprise any number of amino acids, provided that the SARs (or functional portions or functional variants thereof) retain their biological activity, e.g., the ability to specifically bind to antigen, detect diseased cells in a mammal, or treat or prevent disease in a mammal, etc. For example, the SAR can be about 300 to about 5000 amino acids long, such as 300, 400, 500, 600, 700, 800, 900, 1000 or more amino acids in length.

In another aspect, the disclosure relates to an isolated nucleic acid construct comprising at least one nucleic acid encoding a SAR as defined above. In one embodiment, the nucleic acid encodes a protein that targets one of the targets listed in Table B.

Also within the scope of the disclosure are sequences with at least 60%, 70%, 80% or 90% homology to any SAR polypeptide described herein. As SARs are modular in design, additional SAR can be generated by replacing one or more of the modules of the SARs described herein with different modules. For example, the antigen binding domains (i.e., vL, vH, scFv, vHH, FHVH, centyrins etc.) of the SARs can be replaced by antigen binding domains targeting other antigens.

The term “nucleic acid,” “polynucleotide,” or “nucleic acid molecule” refers to deoxyribonucleic acid (DNA) or ribonucleic acid (RNA), or a combination of a DNA or RNA. RNA includes in vitro transcribed RNA or synthetic RNA; an mRNA sequence encoding a SAR polypeptide as described herein). The nucleic acid may further comprise a suicide gene. The construct may be in the form of a plasmid, vector, transcription or expression cassette.

In one embodiment, the vector is an in vitro transcribed vector, e.g., a vector that transcribes RNA of a nucleic acid molecule described herein. The expression vector may be provided to a cell in the form of a viral vector. Viral vector technology is well known in the art and is described, for example, in Sambrook et al. (Molecular Cloning: A Laboratory Manual, Cold Spring Harbor Laboratory, New York, 2013). A number of viral based systems have been developed for gene transfer into mammalian cells. For example, retroviruses such as, adenovirus vectors are used. In one embodiment, a lentivirus vector is used. This is demonstrated in the examples. The disclosure also relates to a virus comprising a SAR described above.

The disclosure also includes an RNA construct that can be directly transfected into a cell. A method for generating mRNA for use in transfection involves in vitro transcription (IVT) of a template with specially designed primers, followed by poly A addition, to produce a construct containing 3′ and 5′ untranslated sequence (“UTR”) (e.g., a 3′ and/or 5′ UTR described herein), a 5′ cap (e.g., a 5′ cap described herein) and/or Internal Ribosome Entry Site (IRES) (e.g., an IRES described herein), the nucleic acid to be expressed, and a poly A tail, typically 50-2000 bases in length (SEQ ID NO: 13-16). RNA so produced can efficiently transfect different kinds of cells. In one embodiment, the template includes sequences for the SAR. In one embodiment, an RNA SAR vector is transduced into a cell, e.g., a T cell, an NK cell or an iPSC, by electroporation. In another embodiment, an RNA SAR vector is transduced into a cell, e.g., a T cell or a NK cell, by causing transient perturbations in cell membrane using a microfluid device. The different chains (or functional polypeptide units) of a SAR can be also introduced in a cell using one or more than one vector a combination of different vectors or techniques.

In another embodiment, one chain or functional polypeptide unit of SAR can be introduced using a retroviral vector while the other functional polypeptide unit is introduced using a lentiviral vector. In another aspect, one functional polypeptide unit is introduced using a lentiviral vector while the other functional polypeptide unit is introduced using a sleeping beauty transposon. In yet another aspect, one functional polypeptide unit is introduced using a lentiviral vector while the other functional polypeptide unit is introduced using RNA transfection. In yet another aspect, one functional polypeptide units is produced in a cell by genetic recombination at the endogeneous TCR chain loci using gene targeting techniques known in the art while the other functional polypeptide unit is introduced using a lentiviral or a retroviral vector.

RNA can be introduced into target cells using any of a number of different methods, for instance, commercially available methods which include, but are not limited to, electroporation, cationic liposome mediated transfection using lipofection, polymer encapsulation, peptide mediated transfection, or biolistic particle delivery systems such as “gene guns” (see, for example, Nishikawa, et al. Hum Gene Ther., 12(8):861-70 (2001) or by causing transient perturbations in cell membranes using a microfluidic device (see, for example, patent applications WO 2013/059343 A1 and PCT/US2012/060646).

In some embodiments, the non-viral method includes the use of a transposon (also called a transposable element).

Exemplary methods of nucleic acid delivery using a transposon include a Sleeping Beauty transposon system (SBTS) and a piggyBac (PB) transposon system.

In some embodiments, cells, e.g., T, NK, NKT, stem cells or iPSC or synthetic T cell, are generated that express a SAR described herein by using a combination of gene insertion using the SBTS and genetic editing using a nuclease (e.g., Zinc finger nucleases (ZFNs), Transcription Activator-Like Effector Nucleases (TALENs), the CRISPR/Cas system, or engineered meganuclease reengineered homing endonucleases).

In some embodiments, use of a non-viral method of delivery permits reprogramming of cells, e.g., T, NK, NKT, stem cells or iPSC or synthetic T cell, and direct infusion of the cells into a subject.

In a further aspect, the disclosure also relates to an isolated cell or cell population comprising one or more nucleic acid construct as described above. The cell has thus been genetically modified to express a SAR nucleic acid construct of the disclosure. Thus, the disclosure provides genetically engineered cells which comprise and stably express a SAR nucleic acid construct of the disclosure. In one embodiment, the cell is selected from the group consisting of a T cell, a Natural Killer (NK) cell, macrophage, granulocyte, dendritic cell, a cytotoxic T lymphocyte (CTL), a regulatory T cell, hematopoietic stem cells and/or pluripotent embryonic/induced stem cells. T cells may be isolated from a patient for transfection with a SAR nucleic acid construct of the disclosure.

For example, cells can be transfected with the nucleic acid of the disclosure ex vivo. Various methods produce stable transfectants which express a SARs of the disclosure. In one embodiment, a method of stably transfecting and re-directing cells is by electroporation using naked DNA. Additional methods to genetically engineer cells using naked DNA encoding a SAR of the disclosure include but are not limited to chemical transformation methods (e.g., using calcium phosphate, dendrimers, liposomes and/or cationic polymers), non-chemical transformation methods (e.g., electroporation, optical transformation, gene electrotransfer and/or hydrodynamic delivery) and/or particle-based methods (e.g., impalefection, using a gene gun and/or magnetofection). The transfected cells demonstrating presence of an integrated un-rearranged vector and expression of the SAR may be expanded ex vivo. Viral transduction methods may also be used to generate redirected cells which express the SAR of the disclosure.

In some embodiments, a vector of the disclosure can further comprise a promoter. Non-limiting examples of a promoter include, for example, an EF-1 promoter, a CMV IE gene promoter, an EF-1α promoter, MNDU3 promoter, an ubiquitin C promoter, a core-promoter or a phosphoglycerate kinase (PGK) promoter. In some embodiments, the promoter is an EF-1 promoter. In further embodiments, the EF-1 promoter comprises SEQ ID NO: 7. In some embodiments, the vector is an RNA nucleic acid. In some embodiments, the vector comprises a poly(A) tail.

In another aspect, the disclosure provides a method of making a cell (e.g., an immune effector cell or population thereof) comprising introducing into (e.g., transducing) a cell, e.g., T, NK, NK cell line, macrophage, NKT, stem cells, iPSC or synthetic T cell described herein, with a vector comprising a nucleic acid encoding a SAR, e.g., a SAR described herein; or a nucleic acid encoding a SAR molecule e.g., a SAR described herein.

The cell can be an immune effector cell (e.g., a T cell, NK cells or a NKT cell, or a combination thereof) or a stem/progenitor cell that can give rise to an immune effector cell or a synthetic T cell. In some embodiment, the cell is an immortalized cell line, such as NK92, NK92MI or a derivative thereof. In some embodiments, the cell in the methods is deficient in constant chains of endogenous T cell receptor α, β1, β2, pre-TCRα, γ or δ or combination thereof. In some embodiments, the cell in the methods is deficient in HLA antigens. In some embodiments, the cell in the methods is deficient in β2 microglobulin. In some embodiments, the cell in the methods is deficient in expression of the target antigen of SAR. For example, the SAR expressing T cell is deficient in endogenous CD5 in case the SAR is directed against CD5 or is deficient in TCR-beta1constant chain in case the SAR is directed against TCR-beta1 constant chain or is deficient in TCR-beta2 constant chain in case the SAR is directed against TCR-beta2 or is deficient in CS1 in case the SAR is directed against CS1.

In some embodiment, the introducing the nucleic acid molecule encoding a SAR comprises transducing a vector comprising the nucleic acid molecule encoding a SAR, or transfecting the nucleic acid molecule encoding a SAR, wherein the nucleic acid molecule is an in vitro transcribed RNA. In some embodiments, the nucleic acid molecule encodes two or more components of a SAR, are introduced by transducing a cell with more than one vector or transfecting with two or more nucleic acid molecules encoding the different subunits of a SAR. For example, a cell may be transduced with two separate vectors each encoding one of the two functional polypeptide units of a SAR. Similarly, a cell may be transduced with two separate in vitro transcribed RNAs each encoding one of the two functional polypeptide units of a SAR. In addition to the functional polypeptide units of the SAR, each of the RNAs may carry a different selection marker or reporter (e.g., tEGFR, tBCMA, or CD34 or CNB30 or mutant DHFR) that can be used to select the cells transduced with both the RNAs and thus expressing both the functional polypeptide units of the SAR.

In an embodiment, a SAR of the disclosure can be expressed using the regulatory elements of an endogenous gene. In an embodiment, the expression cassette encoding the one or more heterologous antigen binding domains of a SAR are targeted to the genetic locus of a naturally occurring signaling receptor or a signaling adaptor. In an embodiment, the one or more heterologous antigen binding domains of a SAR are transcribed in fusion with a mRNA encoding the entire or partial extracellular domain, hinge domain, transmembrane domain and cytosolic domain of the native receptor or signaling adaptor. In an embodiment, the one or more heterologous antigen binding domains of a SAR are transcribed in fusion with a mRNA encoding the hinge domain and cytosolic domain of the native receptor or signaling adaptor. In an embodiment, the one or more heterologous antigen binding domains of a SAR are transcribed in fusion with a mRNA encoding the transmembrane and cytosolic domain of the native receptor or signaling adaptor. In an embodiment, the SAR is expressed under the promoter and transcription regulatory elements of a naturally occurring receptor or a signaling adaptor. In an embodiment, the targeting of SAR to the genetic locus of an endogenous results in disruption of expression of the naturally occurring receptor or signaling adaptor.

Methods to target genes to any specific genetic locus are known in the art. In an embodiment the method involves the use of CRISP/Cas9 or Zn finger nucleases or TALENs. In an embodiment, a SAR expression cassette is targeted to the genetic locus of a native receptor selected from the group of CD16A, CD16B, CD64, CD32, NKp30, NKp44, NKp46, KIR2DL1, KIR2DL2, KIR2DL3, KIR2DL5A, KIR2DL5B, KIR3DL1, KIR3DL2, KIR3DL4, KIR2DL4, KIR2DS1, KIR2DS2, KIR2DS3, KIR2DS4, KIR2DS5, KIR3DS1, NKG2D, NKG2C, NKG2A, NKG2E, NKG2F, DNAM-1, 2B4, OX40, CD28, 4-1BB, CD27, CD81, CD2, CD5, TNFR-I, TNFR-II, Fas, CD30, CD40, CRTAM, TIGIT, CD96, SLAMF6, SLAMF7, CD100, CD160, CEACAM, ILT2, KLRG1, LAIR1 and CD161. In an embodiment, a SAR expression cassette is targeted to the genetic locus of CD3, FcRγ, DAP10 or DAP12. In an embodiment, a SAR expression cassette is targeted to the genetic locus of TAP1, TAP2, tapasin, NLRC5, CIITA, RFXANK, CIITA, RFX5, RFXAP, TCRα or R constant region, NKG2A, NKG2D, CD3ε, CD5, CD52, CD33, CD123, CLL-1, CIS, CBL-B, SOCS2, PD1, CTLA4, LAG3, TIM3, TIGIT, or any gene in the chromosome 6p21 region.

In some embodiments, the method further comprises: a) providing a population of immune effector cells (e.g., T cells or NK cells); and b) removing T regulatory cells from the population, thereby providing a population of T regulatory-depleted cells; wherein steps a) and b) are performed before introducing the nucleic acid encoding the SAR to the population.

In one embodiment, the cell is a human T cell, NK cell, macrophage or dendritic cell. In some embodiments, the cell is a dog cell.

In one embodiment, the cell is a T cell and the T cell is deficient in one or more of endogenous T cell receptor chains. T cells stably lacking expression of a functional TCR according to the disclosure may be produced using a variety of approaches such as use of Zn finger nucleases (ZFN), CRISP/Cas9 and shRNA targeting the endogenous T cell receptor chains.

A T cell lacking a functional endogenous TCR can be, e.g., engineered such that it does not express any functional endogenous TCR on its surface, engineered such that it does not express one or more subunits (e.g., constant chains of endogenous TCRα, TCRβ1, TCRβ2, TCRγ, TCRδ or pre-TCRα) that comprise a functional endogenous TCR or engineered such that it produces very little functional endogenous TCR on its surface. Alternatively, the T cell can express a substantially impaired endogenous TCR, e.g., by expression of mutated or truncated forms of one or more of the subunits of the TCR. The term “substantially impaired TCR” means that this TCR will not elicit an adverse immune reaction in a host.

The disclosure provides SARs based on novel chains (e.g., NKp30, NKp44, NKp46, DAP10, CD3z, NKG2D and CD16 etc.) that are not only expressed and show signaling activity in NK cells but can be also expressed and show signaling activity in T cells and other immune lineages, including monocytes/macrophages, neutrophils and dendritic cells. The disclosure provides a universal method of on a non-T cell, TCR like binding properties, i.e., ability to bind to a peptide/MHC complex. Thus, the disclosure provides a non-T cell with TCR like binding properties. In the embodiment, the disclosure provides that a cell that is not a T cell can be endowed with TCR like binding properties by expressing a SAR of the disclosure where the SAR is a uTCR-SAR comprising variable domains of TCR but lacking a TCR module. In the embodiment, the disclosure provides a simple one step method of conferring TCR like binding properties on any cell without the need of multiple genetic manipulations. In an embodiment, the method involves ectopic expression of a uTCR-SAR in a cell and does not require the additional steps and time expenditure required for ectopic expression of multiple CD3 subunits and selection of high expressing clones. Thus, the method is suitable for conferring T cell like binding and signaling to any cell, including a primary cell, such as a primary NK cell, a hematopoietic stem cell or an iPSC. The method can be also used to confer T cell like binding (i.e., HLA-dependent binding to a peptide antigen) to an antigen and/or TCR like signaling and cytotoxicity on immortalized cell lines, such as NK cell lines (e.g., NK92, NK92MI, NKG and YTS cell lines).

As the SAR (e.g., uTCR-SAR, CD16-SAR etc.) of the disclosure can be expressed in any cell, including a non-T cell, the method of the disclosure has a distinct advantage over other next generation CAR platforms, such as SIR, AbTCR etc., that rely on signaling via the physiological T cell receptor complex. The disclosure provides a SAR that is expressed under a promoter (e.g., EFlu, MNDU3, ubiquitin etc.) that is active in multiple tissues and lineages. The disclosure provides that a SAR of the disclosure can be expressed in a stem cell (e.g., hematopoietic stem cell or iPSC) and differentiation of the stem cell would result in the functional expression of the SAR in multiple lineages, including NK, T, macrophage, basophils, neutrophils, B cell, granulocytes, and dendritic cells, thereby resulting in a strong immune response against the antigen targeted by the SAR. In an embodiment, the SAR is expressed in a hematopoietic stem cell in vivo and the differentiation into different blood lineages occur in vivo. In alternate embodiment of the disclosure, the SAR is expressed in a stem cell (e.g., iPSC) in vitro and the differentiation into different blood lineages occur in vitro. The in vitro differentiated and expanded blood cells of multiple lineages are then administered to a subject.

In some embodiment, the disclosure provides a non-T cell selected from the group of a primary cell (e.g., primary NK cell, g-NK cell, CIK, memory like NK cell, macrophage, dendritic cell, neutrophile, B cell, granulocyte etc.), a cell line, a hematopoietic stem cell, an iPSC cell, an HLA-deficient iPSC cell, an HLA-deficient NK cell, an HLA-deficient NK cell line etc. with TCR like binding properties and/or signaling activities.

In light of the above, the present application provides an iPSC, an iPS cell line cell, or a derivative cell therefrom comprising at least one SAR, wherein the derivative cells are functional effector cells obtained from differentiation of the iPSC comprising SAR. In some embodiments, the derivative cells are hematopoietic cells include, but are not limited to, mesodermal cells with definitive hemogenic endothelium (HE) potential, definitive HE, CD34 hematopoietic cells, hematopoietic stem and progenitor cells, hematopoietic multipotent progenitors (MPP), T cell progenitors, NK cell progenitors, myeloid cells, neutrophil progenitors, T cells, NKT cells, NK cells, B cells, neutrophils, dendritic cells, and macrophages. In some embodiments, the functional derivative hematopoietic cells comprise effector cells such as T, NK, and regulatory cells.

In another embodiment, an iPSC or a derivative cell therefrom comprising SAR, further comprises an exogenous cytokine and/or cytokine receptor comprising at least one of IL2, IL4, IL6, IL7, IL9, IL10, IL11, IL12, IL15, IL18 and IL21.

Additionally provided is an iPSC, an iPS cell line cell, or a derivative cell therefrom comprising at least a SAR expression, may further comprises a polynucleotide encoding at least one exogenous cytokine and/or its receptor (IL) to enable cytokine signaling contributing to cell activation, survival, persistence and/or expansion, wherein the iPSC line is capable of directed differentiation to produce functional derivative hematopoietic cells having improved activation, survival, persistency, expansion, and effector cell function. The exogenously introduced cytokine signaling(s) comprise the signaling of any one, or two, or more of IL2, IL4, IL6, IL7, IL9, IL10, IL11, IL12, IL15, IL18, and IL21. In some embodiments, the cytokine signaling is constitutively activated. In some embodiments, the activation of the cytokine signaling is inducible. In some embodiments, the activation of the cytokine signaling is transient and/or temporal. In some embodiments, the transient/temporal expression of a cell surface cytokine/cytokine receptor is through a retrovirus, Sendai virus, an adenovirus, an episome, mini-circle, or RNAs including mRNA. In some embodiments, the exogenous cell surface cytokine and/or receptor comprised in the SAR overexpressed iPSC or derivative cells thereof enables IL7, IL10, IL15, IL18 or IL21 signaling. In some embodiment, the cytokine is membrane anchored form of IL2, IL15, IL7 etc. An exemplary construct encoding membrane anchored form of hIL2 is represented by SEQ ID NO: 1330. In some embodiments, the cytokine is part of a multipurpose switch.

Also provided is an iPSC, modified HLA-deficient iPCS, an iPS cell line cell, or a derivative cell therefrom comprising a SAR and overexpressed NKG2C, CD94, DAP12, DAP10, BiKE, TriKE, hnCD16, CAR, an IL, a B2M knockout and/or a CITA knockout; and optionally, a polynucleotide encoding HLA-G, wherein the iPSC is capable of directed differentiation to produce functional derivative hematopoietic cells. In one embodiment of the iPSC and its derivative NK or T cell, the cells comprise B2M−/− CIITA−/−, among other genomic editings, and are both HLA-I and HLA-II deficient, wherein the iPSC and its derivative effector cell have improved persistence and/or survival. In some embodiments, the effector cell has increased persistence and/or survival in vivo.

As such, provided herein include an iPSC comprising a SAR, an exogenous cytokine/receptor, a B2M knockout, and a CIITA knockout; wherein when B2M is knocked out, a polynucleotide encoding HLA-G is optionally introduced, and wherein the iPSC is capable of directed differentiation to produce functional derivative hematopoietic cells. Also included in this application are functional iPSC derivative hematopoietic cells comprising overexpressed SAR, an exogenous cytokine/receptor, a B2M knockout, and a CIITA knockout; wherein when B2M is knocked out, a polynucleotide encoding HLA-G is optionally introduced, and wherein the derivative hematopoietic cells include, but are not limited to, mesodermal cells with definitive hemogenic endothelium (HE) potential, definitive HE, CD34 hematopoietic cells, hematopoietic stem and progenitor cells, hematopoietic multipotent progenitors (MPP), T cell progenitors, NK cell progenitors, myeloid cells, neutrophil progenitors, T cells, NKT cells, NK cells, B cells, neutrophils, dendritic cells, and macrophages.

In one embodiment of the cell or the population of the cells comprising said one or more exogenous polynucleotides comprising a SAR, the cell further comprises one or more of the followings: (i) a BiKE or a TriKE; (ii) B2M null or low; (iii) CIITA null or low; (iv) introduced expression of HLA-G or non-cleavable HLA-G; (v) a chimeric antigen receptor (CAR); (vi) a partial or full peptide of a cell surface expressed exogenous cytokine or a receptor thereof, (vii) an accessory module encoding multi-purpose switch; (viii) deletion or reduced expression in at least one of B2M, TAP1, TAP2, tapasin, NLRC5, CIITA, RFXANK, CIITA, RFX5, RFXAP, TCRα or β constant region, NKG2A, NKG2D, CD3ε, CD5, CD52, CD33, CD123, CLL-1, CIS, CBL-B, SOCS2, PD1, CTLA4, LAG3, TIM3, TIGIT, or any gene in the chromosome 6p21 region; and (ix) introduced or increased expression in at least one of HLA-E, 41BBL, CD3ε, CD3γ, CD36, CD3ƒ, FcRγ, DAP10, DAP12, CD4, CD8, CD16, CD47, CD94, CD113, CD131, CD137, CD80, PDL1, A2AR, Fc receptor, an engager, or surface triggering receptor for coupling with bi- or multi-specific or universal engagers.

In an alternate approach to uTCR-SAR, the disclosure also provides a method of expressing a SAR on the SIR, AbTCR, cTCR and other similar platforms that rely on endogenous TCR signaling to be expressed in a non-T cell, such as a NK cell or an NK cell line. In one embodiment, the cell is an immune cell (e.g., NK, monocyte, macrophage, neutrophil, NK92 cell line etc.) or a stem cell (e.g., iPSC) and the immune cell or the stem cell is engineered to ectopically express one or more of CD3ε, CD3γ, CD3δ and CD3ƒ or variants thereof. In one embodiment, the cell is an immune cell or a stem cell (e.g., iPSC) and the immune cell (e.g., NK, monocyte, macrophage, neutrophil etc.) or the stem cell (e.g., iPSC) is engineered to ectopically express one or more CD3ε, CD3γ, and CD3δ or variants thereof. In an embodiment, the cells engineered to ectopically express the CD3ε, CD3γ, CD3δ and CD3ƒ also express one or more SARs of the disclosure. In an embodiment, the SARs of the disclosure comprise one or more TCR constant chains or fragments thereof (e.g., constant chains of TCRα, TCRβ, TCRγ, TCRδ etc.). Exemplary SARs comprising one or more TCR constant chains or fragments include SIR (SEQ ID NO: 2305), cTCR, Ab-TCR (SEQ ID NO: 2309 and 2310), STAR, HIT, TFP and TFPαβ and TFPγδ. In an embodiment, the expression of CD3ε, CD3γ, CD3δ and/or CD3 in a cell facilitates the functional expression of the SAR comprising one or more TCR constant chains or fragments.

In one embodiment, the cell is a stem cell and the stem cell is deficient in one or more of endogenous T cell receptor chains. In another embodiment, the cell is a stem cell in which one or more target antigens (e.g., MPL, CD33, CD123, CD19, etc.) of the SAR have been deleted or mutated to a form that is no longer recognized by the SAR. As an example, a SAR targeting CD19 is expressed in stem cells that have been made deficient in CD19 using CRISP/Cas9 or Zn finger nucleases so that the B cells produced by such stem cells are not eliminated by the T cells expressing the CD19-targeting SAR. Alternatively, a SAR targeting CD19 is expressed in stem cells in which the endogenous CD19 has been mutated to a form that is not targeted by SAR using CRISP/Cas9 or Zn finger nucleases so that the B cells produced by such stem cells are not eliminated by the T cells expressing the CD19-targeting SAR. In another embodiment, the SAR is expressed in immune effector cells and the stem cells from an autologous or an allogeneic donor are genetically engineered to either lack the expression of the SAR-target antigen or to express a mutated form of SAR target antigen which is not recognized by the SAR. For example, a SAR targeting CD19 is expressed in T cells that are infused into a patient along with autologous or allogeneic hematopoietic stem cells that have been made deficient in CD19 using CRISP/Cas9 or Zn finger nucleases so that the B cells produced by such stem cells are not eliminated by the T cells expressing the CD19-targeting SAR. Alternatively, a SAR targeting CD19 is expressed in T cells that are infused into a patient along with autologous or allogeneic hematopoietic stem cells in which the endogenous CD19 has been mutated to a form that is not targeted by SAR using CRISP/Cas9 or Zn finger nucleases so that the B cells produced by such stem cells are not eliminated by the T cells expressing the CD19-targeting SAR. A similar approach can be used to mutate or eliminate other endogenous antigens (e.g., MPL, CD33, CD123 etc.) in stem cells using shRNA, CRISP/Cas9 or Zn finger nucleases in subjects receiving SAR-T cells targeting these antigens for the treatment of specific diseases in which these antigens are expressed on disease associated or disease-causing cells.

Immune cells (e.g., T cells, NK cells, monocytes/macrophages, neutrophils etc.) or stem cells, can be obtained from a subject. The term “subject” is intended to include living organisms in which an immune response can be elicited (e.g., mammals). Examples of subjects include humans, monkeys, chimpanzees, dogs, cats, mice, rats, and transgenic species thereof. Immune cells (e.g., T cells, NK cells, monocytes/macrophages, neutrophils etc.) can be obtained from a number of sources, including peripheral blood mononuclear cells, bone marrow, lymph node tissue, cord blood, thymus tissue, tissue from a site of infection, ascites, pleural effusion, spleen tissue, and tumors. In an embodiment, the immune cells are obtained from a subject who has been administered a mobilization agent, such as a CXCR4 antagonist (e.g., Plerixafor). The immune cell could be tissue resident gamma-delta T cells, which can be cultured and expanded in vitro prior to expression of the SAR.

Immune cells (e.g., T cells, NK cells, monocytes/macrophages, neutrophils etc.) can be obtained by in vitro differentiation of stem cells. In an embodiment, the immune cells are obtained by in vitro differentiation of iPSC.

The immune cells used to express the SAR may be an autologous or an allogeneic immune cell.

Cells that express a SAR of the disclosure, including uTCR-SAR, are used in the treatment of disease.

The disclosure thus relates to methods for the prevention and/or treatment of a disease, such as a cancer, comprising administering to a subject a cell or cell population comprising a SAR as described herein, said method comprising administering, to a subject in need thereof, a pharmaceutically active amount of a cell and/or of a pharmaceutical composition of the disclosure.

The disclosure also relates to a SAR, a cell or cell population comprising a SAR as described herein for use in therapy. The disclosure also relates to a SAR or a cell comprising a SAR as described herein for use in the treatment of cancer. The disclosure also relates to the use of a SAR or a cell comprising a SAR as described herein in the manufacture of a medicament for the treatment of cancer.

In another aspect, the disclosure relates to a method for stimulating a T cell-mediated immune response to a target cell population or tissue in a subject, the method comprising administering to a subject an effective amount of a cell or cell population that expresses a SAR of the disclosure, wherein the antigen binding domain is selected to specifically recognize the target cell population or tissue.

In another aspect, the disclosure relates to a method of providing an anti-tumor immunity in a subject, the method comprising administering to the mammal an effective amount of a cell or cell population genetically modified to express a SAR of the disclosure, thereby providing an anti-tumor immunity in the subject.

In another aspect, the disclosure relates to a method for producing a genetically modified cell or cell population comprising expressing in said cell or cell population a SAR nucleic acid construct of the disclosure. The method may include introducing into the cell a nucleic acid as described herein (e.g., an in vitro transcribed RNA or synthetic RNA; an mRNA sequence encoding a SAR polypeptide as described herein). In embodiments, the RNA expresses the SAR polypeptide transiently. In one embodiment, the cell is a cell as described herein, e.g., an immune effector cell (e.g., T cells or NK cells, or cell population). Cells produced by such methods are also within the scope of the disclosure.

In another aspect, the disclosure relates to an ex vivo method for generating a population of cells for use in adaptive immunotherapy comprising transforming said cell with a SAR of the disclosure.

In certain aspects of the disclosure, immune effector cells, e.g., T cells or NK cells, can be obtained from a unit of blood collected from a subject using any number of techniques known to the skilled artisan, such as Ficoll™ separation. In one embodiment, cells from the circulating blood of an individual are obtained by apheresis. In one aspect, the cells are collected from a subject in whom the T and/or NK cells have been mobilized by administration of an agent. In some embodiments, the immune cells are collected from a donor who has been administered a CXCR4 antagonist (e.g., Plerixafor), a cytokine (e.g., G-CSF, GM-CSF or sargramostim, Neulasta or Pegfilgastrim), a beta2 agonist (e.g., epinephrine), a tyrosine kinase inhibitor (e.g., dasatinib), chemotherapy drug(s) (e.g., cyclophosphamide, doxorubicin etc.) either singly or in combination, prior to the collection of immune cells. In some embodiments, the donor is an autologous donor while in other embodiments, the donor is an allogeneic donor.

The apheresis product usually contains lymphocytes, including T cells, monocytes, granulocytes, B cells, NK cells, other nucleated white blood cells, red blood cells, and platelets. In one aspect, the cells collected by apheresis may be washed to remove the plasma fraction and, optionally, to place the cells in an appropriate buffer or media for subsequent processing steps. In one embodiment, the cells are washed with phosphate buffered saline (PBS). In an alternative embodiment, the wash solution lacks calcium and may lack magnesium or may lack many if not all divalent cations.

In another embodiment, a SAR-expressing effector cell described herein can further express an agent which enhances the activity of a SAR-expressing cell. In some embodiments, the agent is one that inhibits an inhibitory molecule. Non-limiting examples of inhibitory molecules include PD-1, PD-L1, CTLA-4, TIM-3, CEACAM (e.g., CEACAM-1, CEACAM-3 and/or CEACAM-5), LAG-3, VISTA, BTLA, TIGIT, LAIR1, CD160, 2B4 and TGFR beta. In one embodiment, the agent that inhibits an inhibitory molecule comprises a first polypeptide, e.g., a scFv or VHH or a receptor or a ligand fragment that binds an inhibitory molecule, associated with a second polypeptide that provides a positive signal to the cell, e.g., an intracellular signaling domain, such as 41BB, CD27, OX40, CD28, Dap10, CD2, CD5, ICAM-1, LFA-1, Lck, TNFR-1, TNFR-II, Fas, CD30, CD40 or combinations thereof) and/or a primary signaling domain (e.g., a CD3 zeta signaling domain). In one embodiment, the agent that inhibits an inhibitory molecule comprises a first polypeptide, e.g., a scFv or VHH fragment or a receptor or a ligand fragment that binds an inhibitory molecule, associated with a signaling chain described herein (e.g., CD16, NKp30, NKp44, and NKp46 etc.).

In another embodiment, the SAR-expressing cell described herein can further express an accessory module, e.g., an agent which modulate the activity of a SAR-expressing cell. Several examples of accessory modules that comprise of agents that can enhance or regulate the activity of a SAR-expressing cell are provided in SEQ ID NO: 3702-3725. For example, in one embodiment, the agent can be an agent which increases the expression and/or activity of CD3ƒ, CD36, CD3ε, CD3γ or combination thereof. In another embodiment, the agent can be an agent (e.g., vFLIP K13, vFLIP MC159, cFLIP-L, cFLIP-p22, HTLV1 Tax, HTLV2 Tax, 41BB or CD28) which provides costimulatory signal to SAR expressing cells. In another embodiment, the agent can be an agent (e.g., FKBPx2-K13, Myr-MYD88-CD40-Fv′-Fv etc.) which provides costimulatory signal to SAR expressing cells in an inducible manner. In another embodiment, the agent can be a cytokine or a chemokine (e.g., CD40L, IL2, IL-7, IL-15, IL12f or IL-21) that promotes the proliferation or persistence of SAR-expressing cells. In an exemplary embodiment, the agent is membrane anchored form of human IL2 (SEQ ID NO (DNA): 1330 and SEQ ID NO (PRT): 3724).

The disclosure also provides a therapeutic controls/accessory module that serves as a “multi-purpose switch”. In an exemplary embodiment, a multipurpose switch serves as a life-death switch for the purpose of adoptive cell therapy when ectopically expressed in a cell. In an embodiment, the multipurpose switch comprises an in-frame fusion of a first module comprising a receptor-binding domain to a second module that serves as a kill-switch and a third module that serves as a membrane anchoring module. In an embodiment, the first module binds to a receptor that is expressed on cell surface, i.e., it binds to the extracellular domain of a receptor. In an embodiment, the first module binds to a receptor which when bound transmits a pro-survival and/or proliferative signal to the cell. In an embodiment, the first module binds to the receptor in cis (i.e., bind to the receptor expressed on the same cell as the cell expressing the molecular switch). In an embodiment, the first module binds to the receptor in trans (i.e., bind to receptor expressed on a cell other than the cell expressing the molecular switch). In an embodiment, the first module binds to the receptor in cis and in trans. In an embodiment, the second and the third modules are derived from the same endogenous protein. In an embodiment, the second and the third module are derived from different endogenous proteins. In an embodiment, the second module comprises of the extracellular domain of an endogenous protein or a fragment thereof. In an embodiment, the second module can be used to induce death of the cells expressing the molecular switch. In an embodiment, the second module can be used to induce death of the cells expressing the molecular switch when bound by an agent. In an exemplary embodiment, the agent that induces death of cells expressing the molecular switch when bound to the second module is an antibody, a single domain antibody, a non-immunoglobulin antigen binding domain, an antibody drug conjugate, a bispecific antibody or a fragment thereof. In an embodiment, the second module can be used to selectively enrich or deplete cells expressing the molecular switch. In an embodiment, the second module can be used to selectively detect, enrich and/or deplete cells expressing the molecular switch when bound by an agent. In an exemplary embodiment, the agent that can be used to selectively detect, enrich and/or deplete the cells expressing the molecular switch when bound to the second module is an antibody, a single domain antibody, a non-immunoglobulin antigen binding domain or a fragment thereof. In an embodiment, the molecular switch is used to selectively detect, enrich and/or deplete cells ex vivo. In an embodiment, the molecular switch is used to selectively deplete cells in vivo. In an embodiment, the agent (i.e., an antibody, antibody drug conjugate, bispecific antibody, a non-immunoglobulin antigen binding domain or a fragment thereof) that is used to detect, deplete or enrich cells expressing the molecular switch has been approved for human administration by the FDA. Exemplary agents that have been approved by FDA for human administration are known in the art and include, but are not limited to, Rituximab, Herceptin, Erbitrux, adcetris, Enbrel etc. In an embodiment, the agent that is used to detect, deplete or enrich cells expressing the molecular switch is approved by the FDA for ex vivo clinical use. An exemplary such agent is an antibody against CD34 that has been approved by the FDA to be used in conjunction with the clinically approved CliniMACS CD34 system (Miltenyi). An exemplary multipurpose switch is Synth-IL2-Nde-tBCMA-L244ter (SEQ ID NO (DNA):7152 and SEQ ID NO (PRT): 7843) and comprises the IL2 receptor binding domain of IL2 fused in frame to the extracellular-domain and the transmembrane domain of BCMA. This multipurpose switch when expressed in immune cells (e.g., T cells or NK cells etc.) provides them with a survival signal by binding to the IL2 receptor through the N-terminal module comprising IL2. The second module of this multipurpose switch comprises the extracellular domain of BCMA which is recognized by BCMA-binding agents (e.g., BCMA antibodies) and can be used for the detection, selective depletion and/or enrichment of transgene (e.g., SAR) expressing cells. The extracellular domain of BCMA comprising the second module can be also used for selective suicide of transgene (e.g., SAR) expressing cells by the use of BCMA-targeted agents, such as an antibody or an antibody drug-conjugates targeting BCMA. The third module in this molecular switch comprises of the hinge and/or transmembrane domain of BCMA and serves to anchor the switch to the cell membrane. In an embodiment, the second module is a synthetic module comprising one or more copies of an epitope or a mimotope. In an embodiment, the epitope is present in the extracellular domain of an endogenous protein. In an embodiment, the mimotope mimics an epitope that is present in the extracellular domain of an endogenous protein. An exemplary synthetic module comprising one or more copies of an epitope or a mimotope is RQR8, a module harboring a CD34 epitope and two CD20 mimotopes. The RQR8 module allows selection with the clinically approved CliniMACS CD34 system (Miltenyi). Further, the construct binds the widely used pharmaceutical antibody rituximab, resulting in selective deletion of transgene-expressing cells. Additional exemplary multi-purpose molecular switches include fusion proteins comprising IL2 or its variants and tBCMA (SEQ ID NO (DNA): 7151-7155), IL15 or its variants and tBCMA (SEQ ID NO (DNA):7156-7157), IL2 and its variants and tHer2, IL2 and its variants and tEGFR, IL2 and its RQR8 etc. As the multipurpose switches are modular in format, one module can be replaced with a different module. Thus, the IL2 module can be replaced by a different cytokine (e.g., IL15, IL18, IL21 etc.) These multipurpose proteins provide a pro-survival signal through their cytokine moiety (e.g., IL2, IL15, IL18, IL21 etc.) but can be used to kill-off the cells by the use of an agent (e.g., an antibody) that binds to the second module (e.g., RQR3, tBCMA, tHer2, tEGFR, tCD19 etc.), thereby acting as a suicide gene that allow selective deletion of administered T cells in the face of toxicity. The second module (e.g., RQR3, tBCMA, tHer2, tEGFR, tCD19 etc.) can be also used as a marker for measurement of transduction and to allow selection of transduced cells.

In another embodiment, the agent can be an agent that inhibits an inhibitory molecule. Inhibitory molecules, e.g., PD1, can, in some embodiments, decrease the ability of a SAR-expressing cell to mount an immune effector response. In another embodiment, the agent can be a scFV targeting PD1 or CTLA4. In one embodiment, the agent comprises a first polypeptide, e.g., of an inhibitory molecule such as PD1, PD-L1, CTLA4, TIM3, CEACAM (e.g., CEACAM-1, CEACAM-3 and/or CEACAM-5), LAG3, VISTA, BTLA, TIGIT, LAIR1, CD160, 2B4 or TGFR beta, or a fragment of any of these (e.g., at least a portion of an extracellular domain of any of these), and a second polypeptide which is an intracellular signaling domain described herein (e.g., comprising a costimulatory domain (e.g., 41BB, CD27 or CD28, e.g., as described herein) and/or a primary signaling domain (e.g., a CD3 zeta signaling domain described herein). In one embodiment, the agent comprises a first polypeptide of PD1 or a fragment thereof (e.g., at least a portion of an extracellular domain of PD1), and a second polypeptide of an intracellular signaling domain described herein (e.g., a CD28 signaling domain described herein and/or a CD3 zeta signaling domain described herein).

In one embodiment, the SAR-expressing effector cell described herein can further comprise a second SAR that may include a different antigen binding domain to the same or a different target. In some embodiments, the second SAR may target the same or a different cell type from the first SAR.

In one embodiment, the SAR-expressing effector cell described herein can further comprise a second SAR with the same or a different antigen binding domain, optionally the same or a different target. In some embodiments, the second SAR may target the same or a different cell type from the first SAR. The two SARs may have the same backbone or different backbones. In an exemplary embodiment, the two SARs may have the backbone of a CD16 SAR. In another exemplary embodiment, one SAR may have the backbone of a SIR while the second SAR may have the backbone of CD16 SAR. In another exemplary embodiment, one SAR may have the backbone of an Ab-TCR while the second SAR may have the backbone of a CD16 SAR. The nucleic acid and amino acid sequences of several exemplary SARs on different backbones are presented in Tables 32 and 34 of the provisional application. In one embodiment, the SAR includes an antigen binding domain to a target expressed on the same disease cell type (e.g., cancer) as the disease associated antigen. In one embodiment, the SAR expressing cell comprises a first SAR that targets a first antigen, and a 2^{nd} SAR (or a 2^{nd} generation CAR) that targets a second, different, antigen and includes an intracellular signaling domain having no primary signaling domain but a costimulatory signaling domain. While not wishing to be bound by theory, placement of a costimulatory signaling domain, e.g., 4-1BB, CD28, CD27, 2B4 or OX40, onto SAR (e.g., a 2^{nd} generation CAR), can modulate the SAR activity to cells where both targets are expressed. In one embodiment, the SAR expressing cell comprises i) a first disease associated antigen SAR (e.g., a CD16 SAR) that includes one or more antigen binding domains that bind a target antigen described herein, and one or two signaling chains, and ii) a 2^{nd} generation CAR or a TFPε that targets a different target antigen (e.g., an antigen expressed on that same disease associated (e.g., cancer) cell type as the first target antigen) and includes an antigen binding domain, a transmembrane domain and a primary signaling domain and a costimulatory domain. In another embodiment, the SAR expressing cell comprises a i) a first SAR (e.g., a CD16 SAR) that includes an antigen binding domain that binds a target antigen described herein, and one or two signaling chains and ii) a CAR that targets an antigen other than the first target antigen (e.g., an antigen expressed on the same cancer cell type as the first target antigen) and includes an antigen binding domain specific to the antigen, a transmembrane domain and a costimulatory signaling domain. This CAR construct lacks the CD3z domain. In yet another embodiment, the SAR expressing cell comprises i) a first disease associated antigen SAR (e.g., a CD16 SAR) that includes one or more antigen binding domains that bind a target antigen described herein, and one or two signaling chains, and ii) a CAR that targets a different target antigen (e.g., an antigen expressed on that same disease associated (e.g., cancer) cell type as the first target antigen) and includes an antigen binding domain, a transmembrane domain and a primary signaling domain but without a costimulatory domain.

In another exemplary embodiment, an immune cell may express two SARs one of which provides a costimulatory signal while the other provides a primary activation signal. In one embodiment, one SAR may have the backbone of a 4-1BB-based SAR while the second SAR may have the backbone of a CD16-based SAR. In another exemplary embodiment, one SAR may have the backbone of a CD28-based SAR while the second SAR may have the backbone of a CD16-based SAR. In another exemplary embodiment, one SAR may have the backbone of a OX40-based SAR while the second SAR may have the backbone of a CD16-based SAR. In another exemplary embodiment, one SAR may have the backbone of a 2B4 SAR while the second SAR may have the backbone of a CD16-based SAR.

It is to be understood that a SAR that can transmit an activation signal to an immune effector cell but may not have an ITAM containing activation domain. Such SARs may recruit a protein with an ITAM containing activation domain. Exemplary SARs that lack an ITAM containing activation domain include but are not limited to SARs with the backbone of CD16.

In one embodiment, the CAR comprises the antigen binding domain, a transmembrane domain and an intracellular signaling domain (such as but not limited to one or more intracellular signaling domain from 41BB, CD27, 2B4, OX40, CD28, Dap10, CD2, CD5, ICAM-1, LFA-1, Lck, TNFR-1, TNFR-II, Fas, CD30, CD40 or combinations thereof) and/or a primary signaling domain (such as but not limited to a CD3 zeta signaling domain).

In one embodiment, the SAR-expressing effector cell comprises a SAR described herein and an inhibitory CAR. In one embodiment, the inhibitory CAR comprises an antigen binding domain that binds an antigen found on normal cells but not cancer cells. In one embodiment, the inhibitory CAR comprises an antigen binding domain, a transmembrane domain and an intracellular domain of an inhibitory molecule. For example, the intracellular domain of the inhibitory CAR can be an intracellular domain of any one of PD1, PD-L1, CTLA-4, TIM-3, CEACAM (e.g., CEACAM-1, CEACAM-3 and/or CEACAM-5), LAG-3, VISTA, BTLA, TIGIT, LAIR1, CD160, 2B4 or TGFR beta.

In certain embodiments, the antigen binding domain of the first SAR molecule (e.g., CD16 SAR, NKp30 SAR, NKp44 SAR, NKp46 SAR or DAP10 SAR etc.) comprises a scFv and the antigen binding domain of the 2^{nd} SAR molecule (e.g., CD16 SAR, NKp30 SAR, NKp44 SAR, NKp46 SAR or DAP10 SAR etc.) does not comprise a scFv. For example, the antigen binding domain of the first SAR molecule comprises a scFv and the antigen binding domain of the 2^{nd} SAR molecule comprises a camelid VHH domain.

In one embodiment, the disclosure provides an immune effector cell (e.g., T cell, NK cell) expressing a SAR comprising an antigen binding domain that binds to a tumor antigen as described herein, and a CAR comprising a PD 1 extracellular domain or a fragment thereof. In some embodiments, the cell further comprises an inhibitory molecule comprising an inhKIR cytoplasmic domain; a transmembrane domain, e.g., a KIR transmembrane domain; and an inhibitor cytoplasmic domain, e.g., an ITIM domain, e.g., an inhKIR ITIM domain.

The disclosure also provides a method comprising administering a SAR molecule, a cell expressing a SAR molecule or a cell comprising a nucleic acid encoding a SAR molecule to a subject. In one embodiment, the subject has a disorder described herein, e.g., the subject has cancer, infectious disease, allergic disease, degenerative disease or autoimmune disease, which expresses a target antigen described herein. In yet one embodiment, the subject has increased risk of a disorder described herein, e.g., the subject has increased risk of cancer, infectious disease, allergic disease, degenerative disease or autoimmune disease, which expresses a target antigen described herein. In one embodiment, the subject is a human. In another embodiment, the subject is an animal. In yet another embodiment, the subject is a companion animal such as a dog.

In one embodiment, the disclosure provides methods of treating or preventing a disease by providing to the subject in need thereof immune effector cells (e.g., T or NK cells) or stem cells that can give rise to immune effector cells that are engineered to express a targeted X-SIR, wherein X represents a disease associated antigen as described herein, and wherein the disease causing or disease-associated cells express said X antigen. Table 49 provides a list of different antigens and the exemplary diseases that can be prevented, inhibited or treated using immune effector cells expressing SARs targeting these antigens.

In another embodiment, the disclosure provides methods of treating or preventing cancer by providing to the subject in need thereof immune effector cells (e.g., T or NK cells) that are engineered to express a X-targeted SAR (or X-targeted SAR) described herein, wherein the cancer cells express antigen target “X”. In one embodiment, X is expressed on both normal cells and cancers cells but is expressed at lower levels on normal cells. In one embodiment, the method further comprises selecting a SAR that binds X with an affinity that allows the X-targeted SAR to bind and kill the cancer cells expressing X but less than 30%, 25%, 20%, 15%, 10%, 5% or less of the normal cells expressing X are killed, e.g., as determined by an assay described herein. For example, the Gluc release cytotoxicity assay described herein can be used to identify X-targeted SARs that target, e.g., the cancer cells. In one embodiment, the selected SAR has an antigen binding domain that has a binding affinity KD of about 10⁻⁴ M to 10⁻⁸ M, more commonly about 10⁻⁵ M to 10⁻⁷ M, and typically about 10⁻⁶ M or 10⁻⁷ M, for the target antigen. In one embodiment, the selected antigen binding domain has a binding affinity that is at least two-fold, five-fold, 10-fold, 20-fold, 30-fold, 50-fold, 100-fold or 1,000-fold less than a reference antibody, e.g., an antibody described herein and from which the binding domain of the SAR is derived.

In another embodiment, the disclosure provides methods of treating or preventing cancer by providing to the subject in need thereof immune effector cells (e.g., T cells) that are engineered to express a bispecific SAR (or AxB-targeted SAR) described herein, wherein A and B represent the two different antigens of targeted by the SAR. In an embodiment, antigen A is CD19 and antigen B is CD22 and the disease is B cell lymphoma or leukemia. In an embodiment, antigen A is CD19 and antigen B is CD20 and the disease is B cell lymphoma or leukemia. In an embodiment, antigen A is CD19 and antigen B is BCMA and the disease is B cell lymphoma or leukemia. In an embodiment, antigen A is CD19 and antigen B is CD38 and the disease is B cell lymphoma or leukemia. In an embodiment, antigen A is BCMA and antigen B is CD38 and the disease is a plasma cell disorder or primary effusion lymphoma (PEL). In an embodiment, antigen A is BCMA and antigen B is CS1/SLAMF7 and the disease is a plasma cell disorder or primary effusion lymphoma (PEL). In an embodiment, antigen A is CD123 and antigen B is MPL and the disease is acute myeloid leukemia, chronic myeloid leukemia, myeloproliferative disorder or myelofibrosis. n an embodiment, antigen A is CD123 and antigen B is CD33 and the disease is acute myeloid leukemia, chronic myeloid leukemia, or myeloproliferative disorder. In an embodiment, both antigen A and B are expressed on blood cells. In an embodiment, one antigen is expressed preferentially or exclusively on blood lineage cells (e.g., normal B cells or lymphoma cells) while the other antigen is expressed preferentially or exclusively on non-blood cells (e.g., prostate cancer cells). In an exemplary embodiment, antigen A is PSMA and antigen B is CD19 and the disease is prostate cancer. In such a construct targeting of CD19 provides proliferative signal to the SAR cells by targeting of CD19 expressed on the normal B cells while targeting of PSMA induce killing of prostate cancer cells.

In another embodiment, the disclosure provides methods of treating or preventing a disease by providing to the subject in need thereof immune effector cells (e.g., T or NK cells) or stem cells that can give rise to immune effector cells that are engineered to express a CD19xC20 bispecific SAR, wherein the disease causing or disease associated cells express CD19 and CD20. In one embodiment, the disease to be treated or prevented is a cancer or immune disease. In one embodiment, the cancer to be treated or prevented is Acute B cell leukemia, chronic B cell leukemia, or B cell lymphoma.

In another embodiment, the disclosure provides methods of treating or preventing a disease by providing to the subject in need thereof immune effector cells (e.g., T or NK cells) or stem cells that can give rise to immune effector cells that are engineered to express a CD19xC22 bispecific SAR, wherein the disease causing or disease associated cells express CD19 and CD22. In one embodiment, the disease to be treated or prevented is a cancer or immune disease. In one embodiment, the cancer to be treated or prevented is Acute B cell leukemia, chronic B cell leukemia, or B cell lymphoma.

In another embodiment, the disclosure provides methods of treating or preventing a disease by providing to the subject in need thereof immune effector cells (e.g., T or NK cells) or stem cells that can give rise to immune effector cells that are engineered to express a CD19xC22xCD20 trispecific SAR, wherein the disease causing or disease associated cells express CD19, CD22 and CD20. In one embodiment, the disease to be treated or prevented is a cancer or immune disease. In one embodiment, the cancer to be treated or prevented is Acute B cell leukemia, chronic B cell leukemia, or B cell lymphoma.

In another embodiment, the disclosure provides methods of treating or preventing a disease by providing to the subject in need thereof immune effector cells (e.g., T or NK cells) or stem cells that can give rise to immune effector cells that are engineered to express a BCMAxCD38 bispecific SAR, wherein the disease-causing or disease associated cells express BCMA and CD38. In one embodiment, the disease to be treated or prevented is a cancer or immune disease. In one embodiment, the cancer to be treated or prevented is plasma cell disorder (e.g., plasma cell leukemia, myeloma.

In another embodiment, the disclosure provides methods of treating or preventing a disease by providing to the subject in need thereof immune effector cells (e.g., T or NK cells) or stem cells that can give rise to immune effector cells that are engineered to express CD5-SAR, wherein the disease-causing or disease associated cells express CD5. In one embodiment, the disease to be treated or prevented is a cancer or immune disease. In one embodiment, the cancer to be treated or prevented is T cell leukemia or T cell lymphoma. In one embodiment, the immune disorder to be treated or prevented is multiple sclerosis, rheumatoid arthritis, ankylosing spondylitis, inflammatory Bowel Disease, Diabetes Mellitus, Graft vs host disease or autoimmune Thyroiditis.

In another embodiment, the disclosure provides methods of treating or preventing a disease by providing to the subject in need thereof immune effector cells (e.g., T or NK cells) or stem cells that can give rise to immune effector cells that are engineered to express TCRB1-SAR, wherein the disease-causing or disease associated cells express TCRB1 (T cell receptor Beta1 chain). In one embodiment, the disease to be treated or prevented is a cancer or immune disease. In one embodiment, the cancer to be treated or prevented is T cell leukemia or T cell lymphoma. In one embodiment, the immune disorder to be treated or prevented is multiple sclerosis, rheumatoid arthritis, ankylosing spondylitis, inflammatory Bowel Disease, Diabetes Mellitus, Graft vs host disease or autoimmune Thyroiditis.

In another embodiment, the disclosure provides methods of treating or preventing a disease by providing to the subject in need thereof immune effector cells (e.g., T or NK cells) or stem cells that can give rise to immune effector cells that are engineered to express TCRB2-SIR, wherein the disease-causing or disease associated cells express TCRB2 (T cell receptor Beta2 SAR). In one embodiment, the disease to be treated or prevented is a cancer or immune disorder. In one embodiment, the cancer to be treated or prevented is T cell leukemia or T cell lymphoma. In one embodiment, the immune disorder to be treated or prevented is multiple sclerosis, rheumatoid arthritis, ankylosing spondylitis, inflammatory Bowel Disease, Diabetes Mellitus, Graft vs host disease or autoimmune Thyroiditis.

In another embodiment, the disclosure provides methods of treating or preventing a disease by providing to the subject in need thereof immune effector cells (e.g., T or NK cells) or stem cells that can give rise to immune effector cells that are engineered to express T cell receptor gamma-delta-SIR, wherein the disease causing or disease associated cells express T cell receptor gamma-delta. In one embodiment, the disease to be treated or prevented is a cancer or immune disorder. In one embodiment, the cancer to be treated or prevented is T cell leukemia or T cell lymphoma. In one embodiment, the immune disorder to be treated or prevented is multiple sclerosis, rheumatoid arthritis, ankylosing spondylitis, inflammatory bowel disease, diabetes mellitus, Graft vs host disease or autoimmune Thyroiditis.

In another embodiment, the disclosure provides methods of treating or preventing a disease by providing to the subject in need thereof immune effector cells (e.g., T or NK cells) or stem cells that can give rise to immune effector cells that are engineered to express a SAR encoding CD4-DC-SIGN. In one embodiment, the disease to be treated or prevented is HIV1/AIDS.

In another embodiment, the disclosure provides methods of treating or preventing an autoimmune disease by providing to the subject in need thereof immune effector cells (e.g., T or NK cells) or stem cells that can give rise to immune effector cells that are engineered to express a SAR encoding the autoantigen or a fragment thereof. In one embodiment, the autoimmune disease is diabetes mellitus, rheumatoid arthritis, multiple sclerosis, pemphigus vulgaris, paraneoplastic pemphigous, glomerulonephritis, ankylosing spondylitis, Ulcerative Colitis or Crohn's disease. In one aspect, the disease is pemphigus vulgaris, and the antigen binding domain of the SAR comprises of extracellular domain of Desmoglein 3 (Dsg3).

In another embodiment, the disclosure provides methods of treating or preventing a cancer, infection, autoimmune or allergic diseases by providing to the subject in need thereof immune effector cells (e.g., T or NK cells) or stem cells that can give rise to immune effector cells that are engineered to express a SAR encoding the extracellular domain of a naturally occurring receptor (e.g., CD16, CD64, NKp44, NKp30, NKp46, NKG2D etc.) or a fragment thereof along with one or more agents (e.g., an antibody, antibody fragment, antigen binding domain, non-Immunoglobulin antigen binding domain fragment, an autonomous antigen binding domain, a bispecific engager, a bispecific T cell engager or a BiTE, a bispecific Killer engager or a BiKE, a trispecific engager, a trispecific T cell engager, or a trispecific Killer engager or a TriKE etc.) that bind to the naturally occurring receptor comprising the SAR and also bind an antigen expressed on the disease associated cells. In an embodiment, the SAR comprise the extracellular domain of an Fc receptor (e.g., CD16 or CD69 etc.) and SAR-expressing cells are administered with one or more agents (e.g., antibody, an antibody fragment or non-Immunoglobulin antigen binding domain) that binds to the Fc receptor. In an exemplary embodiment, the SAR comprise the extracellular domain of a naturally occurring receptor (e.g., NKp30, NKp44, NKp46, NKG2D or NKG2C) and SAR-expressing cells are administered with one or more agents (e.g., BiKE, TRiKE, BiTE) that bind to the extracellular domain of the naturally occurring receptor. In an exemplary embodiment, the SAR comprises the extracellular domain of NKp46 and is administered with a bispecific killer engager (BiKE) or trispecific killer engager (TRiKE) that binds to NKp46 and CD19 so as to target CD19-expressing cells. In an exemplary embodiment, the SAR comprises the extracellular domain of NKp46 and is administered with a bispecific killer engager (BiKE) that binds to NKp46 and Mesothelin so as to target Mesothelin-expressing cells. In one aspect the disease associated cell is a cancer cell, an infected cell, or a plasma cell or a B cell or a T cell.

In one aspect, the agent (e.g., antibody, BiKE, TRiKE or a fragment thereof) is bound to SAR expressing cells ex vivo prior to administration of SAR cells to the subject. In one aspect, the agent (e.g., antibody, BiKE, TRiKE or a fragment thereof) is administered in vivo. In one aspect, the agent (e.g., antibody, BiKE, TRiKE or a fragment thereof) is administered before, concurrently with or after the infusion of SAR-expressing cells. In one aspect, a single dose of the agent is administered whereas in other aspects multiple doses of the agent (e.g., antibody, BiKE, TRiKE or a fragment thereof) are administered. In one aspect, multiple types of agents are administered. In one aspect, the agent (e.g., antibody, BiKE, TRiKE or a fragment thereof) targets a single antigen. In one aspect, the one or more agents (e.g., antibody, BiKE, TRiKE or a fragment thereof) target multiple antigens. In an exemplary embodiment, the SAR comprises the extracellular domain of NKp44 and a SAR expressing cell is administered with a bispecific killer engager (BiKE) or trispecific killer engager (TRiKE) that binds to NKp46 and CD19 so as to target CD19-expressing cells. In an exemplary embodiment, the SAR comprises the extracellular domain of NKp44 and a SAR expressing cell is administered with a trispecific killer engager (TRiKE) that binds to NKp46 and a co-stimulatory receptor (e.g., CD28 or 4-1BB) on the SAR-expressing effector cells and CD19 on tumor cells so as to target and kill CD19-expressing tumor cells. In an exemplary embodiment, the SAR comprises the extracellular domains of both NKp46 and CD16 and SAR expressing cell is administered with a trispecific killer engager (TRiKE) that binds to NKp46, CD16 and Mesothelin so as to target Mesothelin-expressing cells with increased efficacy. Exemplary BiKE and TRiKE are described in Gauthier L et al, CELL, (2019), 117, 1701. Other BiKE and TRiKE are known in the art and can be used in alternate embodiments of the disclosure. In one aspect the disease associated cell is a cancer cell, an infected cell, or a plasma cell or a B cell or a T cell.

In another embodiment, the disclosure provides methods of treating or preventing a cancer, infection, autoimmune or allergic diseases by providing to the subject in need thereof immune effector cells (e.g., T or NK cells) or stem cells that can give rise to immune effector cells that are engineered to express a universal SAR encoding CD16 or a deletion- or point-mutant fragment thereof along with an antibody or an antibody fragment that binds to the CD16 domain of the SAR and an antigen expressed on the disease associated cells. In one aspect the disease associated cell is a cancer cell, an infected cell, or a plasma cell or a B cell or a T cell.

In another embodiment, the disclosure provides methods of treating or preventing a cancer, infection, autoimmune or allergic diseases by providing to the subject in need thereof immune effector cells (e.g., T cells) or stem cells that can give rise to immune effector cells that are engineered to express a universal SAR encoding an immunoglobulin binding receptor (e.g., CD16, CD64 etc.) or a deletion- or point-mutant fragment thereof. The SAR-expressing immune effector cells are administered to the patient along with one or more antibodies or antibody fragments that bind to the immunoglobulin binding domain of the SAR receptor and to one or more antigens expressed on the disease associated cells. In one aspect, the antibody is bound to SAR expressing cells ex vivo prior to administration of SAR cells to the subject. In one aspect, the antibody is administered in vivo. In one aspect, the antibody or antibody fragment is administered before, concurrently with or after the infusion of SAR-expressing cells. In one aspect, multiple doses of the antibody or antibody fragments are administered. In one aspect, multiple types of antibody or antibody fragments are administered. In one aspect, the antibody or antibody fragments target a single antigen. In one aspect, the antibody or antibody fragments target multiple antigens. An exemplary antibody is Rituximab, Herceptin, Erbitrux etc. In one aspect the disease associated cell is a cancer cell, an infected cell, or a plasma cell or a B cell or a T cell.

In another embodiment, the disclosure provides methods of treating or preventing a cancer, infection, autoimmune or allergic diseases by providing to the subject in need thereof immune effector cells (e.g., T cells) or stem cells that can give rise to immune effector cells that are engineered to express both a universal SAR encoding an immunoglobulin binding receptor or a deletion- or point-mutant fragment thereof joined to a signaling chain described herein (e.g., SEQ ID NO: 3914-3958) and an antigen binding domain (e.g., a scFv, vHH, vL, vH, or a non-immunoglobulin antigen binding domain, BiTE, BiKE, TRiKE). The SARs-expressing immune effector cells are administered to the patient along with one or more antigen binding domains that bind to the immunoglobulin binding domain of the SAR receptor and with one or more antigens expressed on the disease associated cells. In one aspect, the antigen binding domain is bound to SAR expressing cells ex vivo prior to administration of SAR cells to the subject. In one aspect, the antigen binding domain is administered in vivo. In one aspect, the antibody or antibody fragment is administered before, concurrently with or after the infusion of SAR-expressing cells. In one aspect, multiple doses of the antigen binding domain are administered. In one aspect, multiple types of antigen binding domains are administered. In one aspect, the antigen binding domain targets a single antigen. In one aspect, the antigen binding domain targets multiple antigens. In one aspect the disease associated cell is a cancer cell, an infected cell, or a plasma cell or a B cell or a T cell.

In another embodiment, the disclosure provides methods of treating or preventing a cancer, infection, autoimmune or allergic diseases by providing to the subject in need thereof immune effector cells (e.g., T cells, NK cells and/or macrophage etc.) or stem cells that can give rise to immune effector cells that are engineered to express a universal SAR encoding an immunoglobulin receptor or a deletion- or point-mutant fragment thereof thereof along with one or more antibodies or an antibody fragments that bind to the above receptor and one or more antigens expressed on the disease associated cells.

In another embodiment, the disclosure provides methods of treating or preventing a cancer, infection, autoimmune or allergic diseases by providing to the subject in need thereof immune effector cells (e.g., T, NK cells and/or macrophages) or stem cells that can give rise to immune effector cells that are engineered to express both a universal SAR encoding CD16 or a deletion- or point-mutant (e.g., F158V mutant) fragment thereof joined to a T cell receptor constant chain and a SAR encoding an antigen binding domain (e.g., a scFv, vHH, vL, vH, or a non-immunoglobulin antigen binding domain) joined to a T cell receptor constant chain. The SARs-expressing immune effector cells are administered to the patient along with one or more antibody or an antibody fragment that binds to the CD16 domain of the SAR and one or more antigens expressed on the disease associated cells. In one aspect the disease associated cell is a cancer cell, an infected cell, or a plasma cell or a B cell or a T cell. In an embodiment, the antibody is administered in vivo. In an embodiment, the antibody is bound to CD16-SAR cells ex vivo. In an embodiment, multiple infusions of the antibody are administered.

In another embodiment, the disclosure provides methods of treating or preventing a cancer, infection, autoimmune or allergic diseases by providing to the subject in need thereof immune effector cells (e.g., NK or T cells) or stem cells that can give rise to immune effector cells that are engineered to express a universal SAR encoding CD16 or a deletion- or point-mutant fragment (e.g., F158V mutant) thereof along with one or more antibody or an antibody fragments that binds to the CD16 domain of the SAR and one or more antigens expressed on the disease associated cells. In one aspect the disease associated cell is a cancer cell, an infected cell, or a plasma cell or a B cell or a T cell.

In another embodiment, the disclosure provides methods of treating or preventing a cancer, infection, autoimmune or allergic diseases by providing to the subject in need thereof immune effector cells (e.g., NK or T cells) that are engineered to express a SAR encoding NKG2D receptor or a deletion- or point-mutant fragment thereof (e.g., SEQ ID NO: 3407 and 3435). In an embodiment, the NKG2D mutant is NKG2D-AF-G4Sx3-NKG2D-AF (SEQ ID NO: 3407). In an embodiment, the NKG2D mutant is NKG2D-YA-G4Sx3-NKG2D-YA (SEQ ID NO: 3435). In an embodiment, the subject is administered immune effector cells expressing the SAR expressing NKG2D-AF-G4Sx3-NKG2D-AF (SEQ ID NO: 3407) along with protein comprising an antigen binding domain (e.g., scFv, vHH, FHVH etc.) in fusion with ULBP2R. An exemplary protein comprising BCMA-93-FHVH in fusion with ULBP2R is presented in SEQ ID NO: 5431. In another embodiment, the subject is administered immune effector cells expressing the SAR expressing NKG2D-YA-G4Sx3-NKG2D-YA (SEQ ID NO: 3435) along with protein comprising an antigen binding domain (e.g., scFv, vHH, FHVH etc.) in fusion with ULBP2-S3. An exemplary protein comprising BCMA-93-FHVH in fusion with ULBPP2-S3 is presented in SEQ ID NO: 5432. In one aspect the disease associated cell is a cancer cell, an infected cell, or a plasma cell or a B cell or a T cell.

In another embodiment, the disclosure provides methods of treating or preventing a disease by providing to the subject in need thereof immune effector cells (e.g., T or NK cells) or stem cells that can give rise to immune effector cells that are engineered to express CD19-Targeted-SAR. In one aspect the disease is an immune or allergic disease.

In another embodiment, the disclosure provides methods of treating or preventing a disease by providing to the subject in need thereof immune effector cells (e.g., T or NK cells) that are engineered to express CD20-TARGETED-SAR. In one aspect the disease is an immune or allergic disease.

In another embodiment, the disclosure provides methods of treating or preventing a disease by providing to the subject in need thereof immune effector cells (e.g., T or NK cells) that are engineered to express CD22-TARGETED-SAR. In one aspect the disease is an immune or allergic disease.

In another embodiment, the disclosure provides methods of treating or preventing a cancer, infection, autoimmune or allergic diseases by providing to the subject in need thereof immune effector cells (e.g., T or NK cells) or stem cells that can give rise to immune effector cells that are engineered to express a FITC-SAR along with a FITC-labelled antibody or an antibody fragment or an antibody fragment or a receptor or a ligand or a non-Immunoglobulin scaffold that binds to an antigen expressed on the disease associated cells. In one aspect the disease associated cell is a cancer cell, an infected cell, or a plasma cell or a B cell or a T cell.

In another embodiment, the disclosure provides methods of treating or preventing a cancer, infection, autoimmune or allergic diseases by providing to the subject in need thereof immune effector cells (e.g., T or NK cells) or stem cells that can give rise to immune effector cells that are engineered to express an avidin-SAR along with a Biotin-labelled antibody or an antibody fragment or an antibody fragment or a receptor or a ligand or a non-Immunoglobulin scaffold that binds to an antigen expressed on the disease associated cells. In one aspect the disease associated cell is a cancer cell, an infected cell, or a plasma cell.

In another embodiment, the disclosure provides methods of treating or preventing a cancer, infection, autoimmune or allergic diseases by providing to the subject in need thereof immune effector cells (e.g., T or NK cells) or stem cells that can give rise to immune effector cells that are engineered to express an Streptag-SAR along with a Streptag-containing antibody or an antibody fragment or a receptor or a ligand or a non-Immunoglobulin scaffold that binds to an antigen expressed on the disease associated cells. In one aspect the disease associated cell is a cancer cell, an infected cell, or a plasma cell.

In another embodiment, the disclosure provides methods of treating or preventing a disease by providing to the subject in need thereof immune effector cells (e.g., T or NK cells) that are engineered to express IgE-SAR whose antigen binding domain comprises of an antibody or antibody fragment that binds to IgE. In one aspect the disease is an immune or allergic disease.

In another embodiment, the disclosure relates to treatment of a subject in vivo using a PD1-SAR (i.e., a SAR containing the extracellular domain of PD1 as its antigen binding domain) such that growth of cancerous tumors is inhibited. A PD1-SAR may be used alone to inhibit the growth of cancerous tumors. Alternatively, PD1-SAR may be used in conjunction with other SARs, CARs, immunogenic agents, standard cancer treatments, or other antibodies. In one embodiment, the subject is treated with a PD1-SAR and an X-SAR described herein. In another embodiment, a PD1-SAR is used in conjunction with another SAR or CAR, e.g., a SAR or a CAR described herein, and a kinase inhibitor, e.g., a kinase inhibitor described herein.

In another embodiment, the disclosure relates to treatment of a subject in vivo using an X-SAR and a PD1-CAR or a CTL4-CAR such that growth of cancerous tumors is inhibited. In one embodiment, the subject is treated with a PD1-CAR or a CTLA4-CAR and an X-SAR described herein.

Certain cells of the immune system demonstrate cytotoxic activity against particular target cells. Cytotoxic T-lymphocytes express T-cell receptors (TcRs) that are capable of specifically recognizing antigen-derived peptides bound to MHC class I molecules. By contrast, natural killer (NK) cells are not MHC-restricted and do not require antigen presentation by MHC molecules to exert their killing effect. They are able to recognize stressed cells in the absence of peptide-loaded MHC, and to kill cells lacking MHC. NK cells thus play an important role in innate immunity, as these “non-MHC” cells would otherwise not be detected and destroyed by other immune cells.

NK cells (also defined as ‘large granular lymphocytes’) represent a cell lineage differentiated from the common lymphoid progenitor (which also gives rise to B lymphocytes and T lymphocytes). Unlike T-cells, NK cells do not naturally comprise CD3 at the plasma membrane. Importantly, NK cells do not express a TCR and typically also lack other antigen-specific cell surface receptors (as well as TCRs and CD3, they also do not express immunoglobulin B-cell receptors, and instead typically express CD16 and CD56. Thus, NK cells are differentiated by their CD3-, CD56+ phenotype. NK cell cytotoxic activity does not require sensitization but is enhanced by activation with a variety of cytokines including IL-2. NK cells are generally thought to lack appropriate or complete signaling pathways necessary for antigen-receptor-mediated signaling, and thus are not thought to be capable of antigen receptor-dependent signaling, activation and expansion.

A number of T-cell-based therapies for treating cancer have been developed. TCR based cell therapy approaches have shown promise in some studies but suffer from the drawback of MHC restriction, i.e., the TCRs used must be matched to a patient's immune type. Unlike a TCR, a CAR does not need to MHC-matched to the recipient. However, very few cancer-specific surface antigens have thus far been identified which can be used as suitable targets for CARs, and thus the use of CARs in cancer therapies is limited at present. All adoptive cell therapy approaches involving the modification of a T-cell with a TCR or a CAR require the isolation and modification of T-cells from a patient, or from a tissue-type matched donor, which increases the time needed for manufacturing and cost of the procedure. Alternative methods seeking to overcome the above limitations of ACT utilize cytotoxic NK cells, as described for example in WO 98/49268. NK cells, however, lack the expression of CD3ε, y, 6 chains and do not express TCR in their native state. NK92 cell line that has been engineered to ectopically express CD3ε, y, 6 and chains has been shown to support TCR expression. However, this method is cumbersome.

The disclosure provides a novel TCR, designated universal TCR (or uTCR-SAR). In an embodiment, uTCR-SAR is not dependent on CD3 chains for expression and can be expressed in any cell. In an embodiment, uTCR-SAR resembles a physiological TCR in possessing two chains. In an embodiment, the antigen binding domain of uTCR comprises variable domains (Va/Vα and Vb/Vβ or Vg/Vγ and Vd/Vδ) derived from a TCR. In an embodiment, a uTCR lacks the complete TCR constant chains. In an embodiment, one or both chains of a uTCR lack the transmembrane and/or cytosolic domain of a TCR constant chain. In an embodiment, one or both chains of a uTCR lack the hinge, transmembrane and/or cytosolic domain of a TCR constant chain.

The disclosure aims to provide cancer-specific killer cells, based on NK cells, for universal use, meaning that the cells do not have to be matched to the immune type of the subject to be treated, as is presently required for T-cell-based therapies, but they may nonetheless be tailored to the specific immune- and cancer-type of the subject, according to need. Thus, the universal killer cells may be used for personalized medicine.

In an embodiment, the subject is administered different cells expressing SAR of the disclosure. In an embodiment, the SAR is expressed in a stem cell (e.g., hematopoietic stem cell or an iPSC) which is differentiated into multiple different lineages (e.g., T cell, NK cell, macrophage, granulocyte, dendritic cells etc.) of SAR-expressing cells. The natural T cell receptor and several next generation CAR platforms (e.g., SIR, Ab-TCR, HIT, or TFP etc.) can be functionally expressed in only T cells. An advantage of the uTCR-SAR of the disclosure is that it can be expressed in any cell type. In an embodiment, the SAR (e.g., uTCR-SAR) is expressed in a stem cell (e.g., hematopoietic stem cell or an iPSC) which is differentiated ex vivo into multiple different lineages (e.g., T cell, NK cell, macrophage, granulocyte, dendritic cells etc.) of uTCR-SAR-expressing cells. The subject is then administered a population of SAR (e.g., uTCR-SAR) expressing cells. In an alternate embodiment, the SAR (e.g., uTCR-SAR) is expressed in a hematopoietic stem cell, which are then administered to a subject. The SAR expressing hematopoietic stem cells differentiate in vivo into multiple lineages of SAR-expressing immune cells (e.g., T cells, NK cell, macrophage, granulocyte, dendritic cells etc.).

The disclosure also provides that next generation chimeric receptors such as SIR, cTCR, Ab-TCR, TFPαβ and TFPγδ demonstrates poor or negligible expression in NK cells, monocytes/macrophages, dendritic cells and neutrophils. The disclosure provides a method for the expression of SIR, cTCR, Ab-TCR, TFPαβ and TFPγδ in NK cells, NK cell lines (e.g., NK92 and its derivatives), monocytes/macrophages, monocyte cell lines, and neutrophils. The method involves ectopic expression of one or more chains of CD3 complex in NK cells, monocytes/macrophages, dendritic cells and neutrophils. In an alternate embodiment, the method involves ectopic expression of one or more chains of CD3 complex in the stem cells (e.g., iPSC, embryonic stem cells, pluripotent stem cells etc.) that can be differentiated to generate NK cells, monocytes/macrophages, dendritic cells and neutrophils. In an embodiment, the chains of CD3 complex that can be ectopically expressed in NK cells, monocytes/macrophages, dendritic cells, neutrophils and stem cells include CD3ε, CD3γ, CD3δ and CD3ƒ. The SEQ ID NOs of exemplary CD3ε, CD3γ, CD3δ and CD3ƒ chains that can be ectopically expressed in the NK cells, monocytes/macrophages, dendritic cells, neutrophils and stem cells to facilitate the functional expression of SIR, cTCR, Ab-TCR, TFPαβ and TFPγδ are provided in Table 18 of the provisional application. In an embodiment, CD3ε, CD3γ, CD3δ and CD3ƒ chains are ectopically expressed in in the NK cells, monocytes/macrophages, dendritic cells, neutrophils and stem cells to facilitate the functional expression of SIR, cTCR, Ab-TCR, TFPαβ and TFPγδ. In an alternate embodiment, CD3ε, CD3γ, and CD3δ chains are ectopically expressed in in the NK cells, monocytes/macrophages, dendritic cells and neutrophils cells to facilitate the functional expression of SIR, cTCR, Ab-TCR, TFPαβ and TFPγδ. In an embodiment, one of more chains of the CD3 complex are expressed using a single vector. Exemplary vectors are represented by SEQ ID NO: 1331 and 1332. In an alternate embodiment, the one of more chains of the CD3 complex are expressed using more than one vector.

In some aspects, the SAR expressing cells (e.g., immune effector cells, stem cells or iPSC etc.) can be generated using techniques known in the art for generation of different CAR-modified cells (e.g., T cells, NK cells, NKT, g-NK, CIK cells, macrophage, dendritic cells, granulocytes, stem cells, iPSC etc.). Unless stated otherwise, the techniques known in the art for the manufacturing and administration of adoptive cell therapy products can be used for generation of SAR-encoding vectors, for harvesting, isolation and culture of different cell types for genetic modification by SAR-encoding vectors, for expansion, storage, transport, thawing, potency testing, sterility testing and administration of SAR-expressing cells.

A SAR can be introduced into a target cell (e.g., T cell, NK cell, hematopoietic cell etc.) ex vivo and/or in vivo. A SAR expressing effector cell can be expanded ex vivo prior to administration to a subject. In an embodiment, a SAR expressing effector cell can be expanded ex vivo for a period of 2-30 days prior to administration to a subject. The disclosure provides that the SAR-expressing cells can be manufactured using an expansion-free protocol. In an embodiment, the SAR-expressing cells are manufactured over a period of 1 day or 2 days. In an embodiment, the SAR-expressing cells are manufactured over a period of less than 3 days, 4 days, 5 days, 6 days, 7 days, 10 days, 15 days, 21 or 30 days. A SAR expressing effector cell can be cryopreserved for storage and then thawed prior to administration.

In another aspect of the disclosure, there is provided a pharmaceutical composition comprising a SAR or an isolated cell or cell population comprising a SAR according to the disclosure and optionally a pharmaceutically acceptable carrier.

The genetically modified cells or pharmaceutical composition of the disclosure can be administered by any convenient route, including parenteral administration. Parenteral administration includes, for example, intravenous, intramuscular, intraarterial, intraperitoneal, intranasal, rectal, intravesical, intradermal, topical or subcutaneous administration. Compositions can take the form of one or more dosage units.

The SAR expressing immune cells can be administered to an individual by absolute numbers of cells, e.g., said individual can be administered from about 1000 cells/injection to up to about 10 billion cells/injection, such as at about, at least about, or at most about, 1×10⁸, 1×10⁷, 5×10⁷, 1×10⁶, 5×10⁶, 1×10⁵, 5×10⁵, 1×10⁴, (and so forth) SAR cells per injection, or any ranges between any two of the numbers, end points inclusive. In other embodiments, SAR expressing cells can be administered to such an individual by relative numbers of cells, e.g., said individual can be administered about 1000 cells to up to about 10 billion cells per kilogram of the individual, such as at about, at least about, or at most about, 1×10⁸, 1×10⁷, 5×10⁷, 1×10⁶, 5×10⁶, 1×10⁵, 5×10⁵, 1×10⁴ (and so forth) cells per kilogram of the individual, or any ranges between any two of the numbers, end points inclusive. SAR cells can also be administered to such a patient according to an approximate ratio between a number of SAR cells and the size of the tumor in said patient. The size of the tumor can be determined or estimated by conventional imaging methods, such X-ray, ultrasound imaging, or the like. In other embodiments, the total dose may be calculated by m² of body surface area, including 1×10⁸, 1×10⁷, 5×10⁷, 1×10⁶ per m². The average person is 1.6-1.8 m².

The SAR expressing immune cells (e.g., NK92 cell line) can be irradiated prior to administration. The SAR expressing immune cells can be treated with an agent (e.g., Mitomycin-C) that makes them replication-incompetent prior to administration to the subject.

The SAR-expressing cells, and optionally other anti-tumor agents, can be administered once to a patient or can be administered multiple times, e.g., once every 1, 2, 3, 4, 5, 6, 7, 8, 9, 10, 11, 12, 13, 14, 15, 16, 17, 18, 19, 20, 21, 22 or 23 hours, or once every 1, 2, 3, 4, 5, 6 or 7 days, or once every 1, 2, 3, 4, 5, 6, 7, 8, 9, 10 or more weeks during therapy, or any ranges between any two of the numbers, end points inclusive.

The SAR-expressing cells can be administered by different routes known in the art, such as intravenous, intraperitoneal, intra-pleural, intrathecal, intraventricular, intra-dermal, subcutaneous, intra-tumoral, intra-lesional, intrahepatic routes etc.

In some embodiments of the SAR-expressing cells are administered in combination with other therapies including conventional therapy, such as chemotherapy, radiotherapy, biological therapy, antibody therapy or hormone treatment. In an embodiment, the SAR expressing cells are administered to a subject after the subject has received lymphodepleting chemotherapy. In an embodiment, the SAR expressing cells are administered to a subject after the subject has received both lymphodepleting and myelodepleting chemotherapy. In an embodiment, the SAR expressing cells are administered to a subject after the subject has received chemotherapy comprising etoposide. In an embodiment, SAR-expressing cells are administered with an agent selected from one or more of the following: a protein phosphatase inhibitor; a kinase inhibitor (e.g., src kinase inhibitor); a Lck kinase inhibitor (e.g., Dasatinib); agents that bind to one or more antigens expressed on the SAR-expressing effector cell and one or more antigens expressed on a target cell (e.g., an antibody, an antibody fragment, a BiTE, a BiKE, a TRiKE etc.); a cytokine (e.g., IL2, IL15 etc.); an inhibitor of an immune inhibitory molecule (e.g., a PD1 or PDL1 inhibitor); an agent that decreases the level or activity of a TREG cell (e.g., rapamycin); an agent that increase the proliferation and/or persistence of SAR-modified cells (e.g., IL2, IL15, IL18, IL21 etc.); a chemokine; an agent that increases the expression of SAR; an agent that allows regulation of the expression or activity of SAR; an agent that allows control over the survival and/or persistence of SAR-modified cells; an agent that controls the side effects of SAR-modified cells (e.g., Tocilizumab, Anakinra, steroid, dasatinib, ibrutinib etc.); a Brd4 inhibitor; an agent that delivers a therapeutic or prophylactic agent to the site of the disease; an agent that increases the expression of the target antigen against which SAR is directed (e.g., a y secretase inhibitor when used with a BCMA targeted SAR); an agent that binds to a multipurpose switch co-expressed with the SAR (e.g., Rituximab, Herceptin or); an agent that protects allogeneic SAR cells from immune attack (e.g., a CD52 antibody) and an adenosine A2a receptor antagonist.

In one embodiment, the method includes administering a cell expressing the SAR molecule, as described herein, in combination with an agent which enhances the activity of a SAR-expressing cell, wherein the agent is a cytokine, e.g., IL-2, IL-7, IL-15, IL-21, or a combination thereof. The cytokine can be delivered in combination with, e.g., simultaneously or shortly after, administration of the SAR-expressing cell. Alternatively, the cytokine can be delivered after a prolonged period of time after administration of the SAR-expressing cell, e.g., after assessment of the subject's response to the SAR-expressing cell. In one embodiment the cytokine is administered to the subject simultaneously (e.g., administered on the same day) with or shortly after administration (e.g., administered 1 day, 2 days, 3 days, 4 days, 5 days, 6 days, or 7 days after administration) of the cell or population of cells of any of claims 143 to 161. In other embodiments, the cytokine is administered to the subject after a prolonged period of time (e.g., at least 2 weeks, 3 weeks, 4 weeks, 6 weeks, 8 weeks, 10 weeks, or more) after administration of the cell or population of cells, or after assessment of the subject's response to the cell.

The disclosure provides that Src inhibitors (e.g., Lck inhibitor, e.g., Dasatinib, ponatinib etc.) can control the activity of the SAR of the disclosure. In an embodiment, Dasatinib can control the activity of a SAR comprising the transmembrane and/or cytosolic domains of CD3z, CD16, NKp30, NKp44, NKp46, or NKG2D etc. In one embodiment, the disclosure provides that an agent that can be administered to treat the side effects of the novel SAR-expressing cells of the disclosure is a Src inhibitor (e.g., Lck inhibitor, e.g., Dasatinib). In one embodiment, Dasatinib is administered to the patient after the administration of SAR-expressing cells to control or terminate the activity of SAR-expressing cells. In one embodiment, Dasatinib is administered to a subject to prevent and/or treat Cytokine Release Syndrome (CRS) caused by SAR-expressing cells. In one embodiment, Dasatinib is administered to a subject to prevent and/or treat neurological complications caused by SAR-expressing cells. In an embodiment, Dasatinib is used in combination with other agents (e.g., steroids, Tocilizumab, Anakinra etc.) to prevent and/or treat CRS and neurological complications caused by SAR-expressing cells.

In one embodiment, dasatinib is administered orally at a dose of at least 10 mg/day, 20 mg/day, 40 mg/day, 60 mg/day, 70 mg/day, 90 mg/day, 100 mg/day, 140 mg/day, 180 mg/day, 210 mg/day, 250 mg/day or 280 mg/day. In one embodiment, the disclosure provides that an agent that can be administered to treat the side effects of the novel SAR-expressing cells of the disclosure is Ponatinib. In an embodiment, Ponatinib is administered to the patient after the administration of CAR-expressing cells to control or terminate the activity of CAR-expressing cells. In one embodiment, ponatinib is administered orally at a dose of at least 15 mg/day, 30 mg/day, 45 mg/day, 60 mg/day. In one embodiment, Ponatinib is administered to a subject to prevent and/or treat Cytokine Release Syndrome (CRS) caused by SAR-expressing cells. In one embodiment, Ponatinib is administered to a subject to prevent and/or treat neurological complications caused by SAR-expressing cells. In an embodiment, Ponatinib is used in combination with other agents (e.g., steroids, Tocilizumab, Anakinra etc.) to prevent and/or treat CRS and neurological complications caused by SAR-expressing cells.

In an embodiment, the disclosure provides that a tyrosine kinase inhibitor can be used to prolong the persistence of SAR-expressing cells of the disclosure with novel signaling domains. In an embodiment, the disclosure provides that a tyrosine kinase inhibitor can be used to prolong the persistence of SAR-expressing cells of the disclosure with novel signaling domains where the SAR comprises the transmembrane and/or cytosolic domain of CD3z, CD16A, CD16B, NKp30, NKp44, NKp46, and/or NKG2D etc. In an embodiment, the disclosure provides that a tyrosine kinase inhibitor can be used to delay/reverse exhaustion of SAR-expressing cells of the disclosure with novel signaling domains where the SAR comprises the transmembrane and/or cytosolic domains of CD3z, CD16, NKp30, NKp44, NKp46, and/or NKG2D etc. In an embodiment, the tyrosine kinase inhibitor is a Src kinase inhibitor. In an embodiment, the Src kinase inhibitor is an Lck inhibitor. In an embodiment, a Lck inhibitor is Dasatinib or Ponatinib. In an embodiment, Dasatinib can be used to prevent/delay the exhaustion of SAR-expressing cells of the disclosure. In an embodiment, the SAR comprises the transmembrane and/or cytosolic domain of CD3z, CD16, NKp30, NKp44, NKp46, and/or NKG2D etc. In an embodiment, Dasatinib can be used to reverse the exhaustion of SAR-expressing cells of the disclosure. In an embodiment, the SAR comprises the transmembrane and/or cytosolic domain of CD3z, CD16A, CD16B, NKp30, NKp44, NKp46, and/or NKG2D etc. In an embodiment, Dasatinib is administered to a subject who has received SAR-expressing cells intermittently. In an embodiment, the subject receives multiple cycles of a Tyrosine kinase inhibitor (e.g., dasatinib). In an embodiment, treatment with a tyrosine kinase inhibitor (e.g., Dasatinib or Ponatinib) prevents apoptosis of SAR-expressing cells of the disclosure. In an embodiment, with a tyrosine kinase inhibitor (e.g., Dasatinib or Ponatinib) decreases the expression of at least one T or NK cell exhaustion marker selected from the group consisting of PD1, TIM-3, and LAG-3. In an embodiment, treatment with a tyrosine kinase inhibitor (e.g., Dasatinib or Ponatinib) increases the expression of CD62L or CCR7 on SAR-expressing cells. In an embodiment, treatment is continued for a time sufficient to restore at least partial function of SAR expressing cells (e.g., T cells or NK cells). In an embodiment, a tyrosine kinase inhibitor (e.g., Dasatinib or Ponatinib) is administered to a subject who has received SAR-expressing cells continuously. In embodiments, Dasatinib is administered at a dose of about 10 mg/day to 240 mg/day (e.g., 10 mg/day, 20 mg/day, 40 mg/day, 50 mg/day, 70 mg/day, 80 mg/day, 100 mg/day, 110 mg/day, 120 mg/day, 140 mg/day, 180 mg/day, 210 mg/day, 240 mg/day or 300 mg/day).

In an embodiment, Ponatinib can be used to prolong the persistence of SAR-expressing cells of the disclosure. In an embodiment, Ponatinib can be used to delay the exhaustion of SAR-expressing cells of the disclosure. In an embodiment, Ponatinib is administered to a subject who has received SAR-expressing cells intermittently. In an embodiment, Ponatinib is administered to a subject who has received SAR-expressing cells continuously.

The composition of the disclosure can be in the form of a liquid, e.g., a solution, emulsion or suspension. The liquid can be useful for delivery by injection, infusion (e.g., IV infusion) or sub-cutaneously. The liquid compositions of the disclosure, whether they are solutions, suspensions or other like form, can also include one or more of the following: sterile diluents such as water, saline solution, typically physiological saline, Ringer's solution, isotonic sodium chloride, fixed oils such as synthetic mono or diglycerides, polyethylene glycols, glycerin, or other solvents; antibacterial agents such as benzyl alcohol or methyl paraben; and agents for the adjustment of tonicity such as sodium chloride or dextrose. A composition can be enclosed in an ampoule, a disposable syringe or a multiple-dose vial made of glass, plastic or other material.

The amount of the pharmaceutical composition of the disclosure that is effective/active in the treatment of a particular disorder or condition will depend on the nature of the disorder or condition and can be determined by standard clinical techniques. In addition, in vitro or in vivo assays can optionally be employed to help identify optimal dosage ranges. The precise dose to be employed in the compositions will also depend on the route of administration, and the seriousness of the disease or disorder, and should be decided according to the judgment of the practitioner and each patient's circumstances.

The compositions of the disclosure comprise an effective amount of a binding molecule of the disclosure such that a suitable dosage will be obtained. The correct dosage of the compounds will vary according to the particular formulation, the mode of application, and its particular site, host and the disease being treated. Other factors like age, body weight, sex, diet, time of administration, rate of excretion, condition of the host, drug combinations, reaction sensitivities and severity of the disease shall be taken into account. Administration can be carried out continuously or periodically within the maximum tolerated dose.

Typically, this amount is at least about 0.01% of a binding molecule of the disclosure by weight of the composition.

Typical compositions of the disclosure are prepared so that a parenteral dosage unit contains from about 0.01% to about 2% by weight of the binding molecule of the disclosure.

For intravenous administration, the composition can comprise from about typically about 0.1 mg/kg to about 250 mg/kg of the animal's body weight, typically, between about 0.1 mg/kg and about 20 mg/kg of the animal's body weight, and more commonly about 1 mg/kg to about 10 mg/kg of the animal's body weight.

The present compositions can take the form of suitable carriers, such aerosols, sprays, suspensions, or any other form suitable for use. Other examples of suitable pharmaceutical carriers are described in “Remington's Pharmaceutical Sciences” by E. W. Martin.

The pharmaceutical compositions can be prepared using methodology well known in the pharmaceutical art. For example, a composition intended to be administered by injection can be prepared by combining a binding molecule of the disclosure with water so as to form a solution. A surfactant can be added to facilitate the formation of a homogeneous solution or suspension.

The pharmaceutical composition of the disclosure can be co-administered with other therapeutics, for example anti-cancer agents, chemotherapy drugs, surgery or radiation.

In one embodiment, the cancer is selected from a hematological cancer or malignancy or a solid tumor. In one embodiment, the cancer is metastatic. In an embodiment cancer is any cancer of any organ or tissue. The exemplary diseases and the antigens targeted by the SAR of the disclosure is presented in Table 49. In an exemplary embodiment, the SAR is used to target BCMA and CD38 and is used to treat plasma cell and autoimmune disorders, such as multiple myeloma, plasma cell leukemia, primary effusion lymphoma and systemic lupus erythematosus (SLE). Such a SAR includes antigen binding domains specific to BCMA and CD38 as described herein. In another exemplary embodiment, the SAR is used to target BCMA and CD22 and is used to treat lymphoid disorders and plasma cell disorders, such as lymphoma, acute lymphocytic leukemia, multiple myeloma, plasma cell leukemia primary effusion lymphoma and systemic lupus erythematosus (SLE). Such a SAR includes antigen binding domains specific to BCMA and CD22 as described herein. In one embodiment, the SAR is used to target CD19, CD22, BCMA and CD20 and is used to treat plasma cell, lymphoid and autoimmune disorders, such as multiple myeloma, plasma cell leukemia, primary effusion lymphoma, diffuse large B cell lymphoma, acute lymphocytic leukemia, chronic lymphocytic leukemia and systemic lupus erythematosus (SLE). Such a SAR includes antigen binding domains specific to CD19, CD22, BCMA and CD20 as described herein. In one embodiment, the SAR is used to target PSMA and CD19 and treat prostate cancer. Such a SAR includes antigen binding domains specific to PSMA and CD19 as described herein. The CD19 specific antigen binding domain is used to primarily stimulate activation, proliferation and expansion of the SAR.

In an embodiment, the SAR of the disclosure is used to treat any disease (e.g., infection, allergy, autoimmune, degenerative disease etc.).

In one embodiment, the SAR is used to target BCMA and CD19 and is used to treat lymphoid disorders and plasma cell disorders, such as lymphoma, acute lymphocytic leukemia, multiple myeloma, plasma cell leukemia primary effusion lymphoma and systemic lupus erythematosus (SLE). Such a SAR includes antigen binding domains specific to BCMA and CD19 as described herein.

In one embodiment, the SAR is used to target BCMA, CD38 and CD22 and is used to treat lymphoid, plasma cell and autoimmune disorders, such as multiple myeloma, plasma cell leukemia, primary effusion lymphoma, diffuse large B cell lymphoma, B-ALL, chronic lymphocytic leukemia and systemic lupus erythematosus (SLE). Such a SAR includes antigen binding domains specific to BCMA, CD38 and CD22 as described herein.

In one embodiment, the SAR is used to target BCMA, CD38 and CD19 and is used to treat lymphoid, plasma cell and autoimmune disorders, such as multiple myeloma, plasma cell leukemia, primary effusion lymphoma, diffuse large B cell lymphoma, B-ALL, chronic lymphocytic leukemia and systemic lupus erythematosus (SLE). Such a SAR includes antigen binding domains specific to BCMA, CD38 and CD19 as described herein.

In one embodiment, the SAR is used to target BCMA, CD3ε, CD22 and CD19 and is used to treat lymphoid, plasma cell and autoimmune disorders, such as multiple myeloma, plasma cell leukemia, primary effusion lymphoma, diffuse large B cell lymphoma, B-ALL, chronic lymphocytic leukemia and systemic lupus erythematosus (SLE). Such a SAR includes antigen binding domains specific to BCMA, CD3ε, CD22 and CD19 as described herein.

In one embodiment, the SAR is used to target BCMA, CD3ε, CD22, CD20 and CD19 and is used to treat lymphoid, plasma cell and autoimmune disorders, such as multiple myeloma, plasma cell leukemia, primary effusion lymphoma, diffuse large B cell lymphoma, B-ALL, chronic lymphocytic leukemia and systemic lupus erythematosus (SLE). Such a SAR includes antigen binding domains specific to BCMA, CD3ε, CD22, CD20 and CD19 as described herein.

In one embodiment, the SAR is used to target CD19 and CD22 and is used to treat lymphoid and autoimmune disorders, such as lymphoma, acute lymphocytic leukemia, chronic lymphocytic leukemia and systemic lupus erythematosus (SLE). Such a SAR includes antigen binding domains specific to CD19 and CD22 as described herein.

In one embodiment, the SAR is used to target CD19 and CD20 and is used to treat lymphoid and autoimmune disorders, such as lymphoma, acute lymphocytic leukemia, chronic lymphocytic leukemia and systemic lupus erythematosus (SLE). Such a SAR includes antigen binding domains specific to CD19 and CD22 as described herein.

In one embodiment, the SAR is used to target CD19, CD22 and CD20 and is used to treat lymphoid and autoimmune disorders, such as lymphoma, acute lymphocytic leukemia, chronic lymphocytic leukemia and systemic lupus erythematosus (SLE). Such a SAR includes antigen binding domains specific to CD19, CD20 and CD22 as described herein.

In one embodiment, the SAR is used to target PSMA and treat prostate cancer. Such a SAR includes an antigen binding domain specific to PSMA as described herein.

In one embodiment, the SAR is used to target PSMA and CD22 and treat prostate cancer. Such a SAR includes antigen binding domains specific to PSMA and CD22 as described herein. The CD22 specific antigen binding domain is used to primarily stimulate activation, proliferation and expansion of the SAR.

In one embodiment, the SAR is used to target PSMA and CD20 and treat prostate cancer. Such a SAR includes antigen binding domains specific to PSMA and CD20 as described herein. The CD20 specific antigen binding domain is used to primarily stimulate activation, proliferation and expansion of the SAR.

In one embodiment, the SAR is used to target PSMA and BCMA and treat prostate cancer. Such a SAR includes antigen binding domains specific to PSMA and BCMA as described herein. The BCMA specific antigen binding domain is used to primarily stimulate activation, proliferation and expansion of the SAR.

In one embodiment, a SAR or cell comprising a SAR of the disclosure is used in combination with an existing therapy or therapeutic agent, for example an anti-cancer therapy. Thus, in another aspect, the disclosure also relates to a combination therapy comprising administration of a SAR-T or SAR-NK and/or SAR-macrophage or pharmaceutical composition of the disclosure and an anti-cancer therapy. The anti-cancer therapy may include a therapeutic agent or radiation therapy and includes gene therapy, viral therapy, RNA therapy bone marrow transplantation, nanotherapy, targeted anti-cancer therapies or oncolytic drugs. Examples of other therapeutic agents include other checkpoint inhibitors, antineoplastic agents, immunogenic agents, attenuated cancerous cells, tumor antigens, antigen presenting cells such as dendritic cells pulsed with tumor-derived antigen or nucleic acids, immune stimulating cytokines (e.g., IL-2, IFNa2, GM-CSF), targeted small molecules and biological molecules (such as components of signal transduction pathways, e.g., modulators of tyrosine kinases and inhibitors of receptor tyrosine kinases, and agents that bind to tumor-specific antigens, including EGFR antagonists), an anti-inflammatory agent, a cytotoxic agent, a radiotoxic agent, or an immunosuppressive agent and cells transfected with a gene encoding an immune stimulating cytokine (e.g., GM-CSF), chemotherapy. In one embodiment, the SAR-T or SAR-NK and/or SAR-T and/or SAR-macrophage pharmaceutical composition of the disclosure is used in combination with surgery. The SAR-T or SAR-NK pharmaceutical composition of the disclosure may be administered at the same time or at a different time as the other therapy, e.g., simultaneously, separately or sequentially.

Immune cell (e.g., NK, T cells etc.) survival and, hence, cytotoxicity requires cytokine support. Expression of interleukin-15 (IL-15) and IL-2 in a non-secretory, membrane-bound form is known to sustain NK and T cell growth and improve cytotoxicity.

The disclosure provides a method of producing an immune cell that expresses all or a functional portion of low-affinity variants of IL-2 and/or IL-15. All or a portion of the IL-2 and/or IL-15 can be expressed as a membrane-bound polypeptide, a secreted polypeptide or as a combination thereof. The method comprises introducing nucleic acid encoding all or a functional portion of low affinity variants of IL-2 or IL-15 into the one or more immune cells (e.g., NK cells). In one aspect, the nucleic acid encoding all or a functional portion of low affinity variants of IL-2 or IL-15 is linked (e.g., fused) to all or a portion of a transmembrane protein. Alternatively, or in addition, nucleic acid encoding all or a functional portion of IL-2 or IL-15 variants is introduced into the immune cells (e.g. NK cell). As will be apparent to those of skill in the art, aspects in which nucleic acid encoding all or a functional portion of IL-2 or IL-15 variant and all or a functional portion of IL-2 or IL-15 variant fused to all or a portion of a transmembrane protein is introduced in to an immune cell (e.g., NK cell, can be done so using a single nucleic acid or multiple (e.g., separate; two) nucleic acids. The NK or T cell is maintained under conditions in which all or a functional portion of the IL-15 or IL-2 is expressed as a membrane-bound polypeptide and/or as a secreted polypeptide thereby producing a NK or T cell that expresses all or a functional portion of IL-15 or IL-2 as a membrane-bound polypeptide and/or as a secreted polypeptide. In a particular aspect, nucleic acid encoding all or a functional portion of IL-15 or IL-2 is fused to a signal peptide of CD8a and all or a portion of a transmembrane domain of CD8a is introduced into the NK cell.

In yet another aspect, the disclosure is directed to a method of enhancing expansion and/or survival of NK cells (e.g., in vitro, ex vivo, and/or in vivo). The method comprises introducing nucleic acid encoding all or a functional portion of IL-15 or IL-2. Nucleic acid encoding all or a portion of the IL-15 (e.g., wild type IL-15) and/or encoding all or a functional portion of IL-15 fused to all or a portion of a transmembrane protein can be introduced into the NK cell. Thus, the NK cell can express all or a functional portion of IL-15 as a membrane-bound polypeptide, a secreted polypeptide or as a combination thereof. The NK cells are maintained under conditions in which all or a portion of the IL-15 is expressed as a membrane-bound polypeptide, a secreted polypeptide or as a combination thereof and in which the NK cells proliferate. In a particular aspect, nucleic acid encoding all or a functional portion of IL-15 is fused to a signal peptide of CD8a and all or a portion of a transmembrane domain of CD8a is introduced into the NK cell. In some aspects, the method can further comprise contacting the NK cells comprising membrane-bound IL-15 and/or secreted IL-15 with IL-2. In some aspects, the concentration of IL-2 is from about 10 IU/ml to about 1000 IU/ml. In other aspects, the concentration of IL-2 is about 20, 40, 60, 80,100,120, 140, 160, 180, 200, 220, 240, 260, 280, 300, 320, 340, 360, 380, 400, 420, 440, 460, 480, 500, 520, 540, 560, 580, 600, 620, 640, 660, 680, 700, 720 740, 760, 780, 800, 820, 840, 860, 880, 900, 920, 940, 960, 980 IU/ml.

As will be apparent to those of skill in the art, a variety of methods for introducing nucleic acid can be used.

Also apparent to those of skill in the art is that a variety of methods of maintaining immune cells (e.g. NK cells) under conditions in which (i) all or a functional portion of the IL-15 is expressed as a membrane-bound polypeptide and/or as a secreted polypeptide and/or (ii) the NK cells comprising membrane-bound IL-15 and/or secreted IL-15 proliferate can be used. The methods can further comprise isolating or separating the one or more NK cells produced by the methods provided herein, In addition, the methods can further comprise culturing the one or more NK cells. In some aspects, an NK cell line is produced.

The disclosure also encompasses a (one or more) natural killer (NK) cell or cell line produced by the methods described herein, and compositions comprising the NK cells provided herein. In a particular aspect, the composition is a pharmaceutical composition comprising one or more of the NK cells or cell lines provided herein. The pharmaceutical composition can further comprise all or a functional portion of IL-2 (e.g., all or a functional portion of an (one or more) IL-2 protein; nucleic acid encoding all or a functional portion of IL-2).

The disclosure provides membrane anchored form of IL2 and IL15 and their low affinity variants (Table 43) that can be used to provide IL2 and/or IL15 signaling to immune cells (e.g., T cells, NK cells, NK-T cells), including immune cells expressing the SARs of the disclosure (e.g., SEQ ID NO: 8428). The SEQ ID NO: of representative low affinity IL2 variants are provided in SEQ ID NO: 7834-7837. The SEQ ID NO: of representative low affinity IL15 variants are provided in SEQ ID NO: 7839-7841. The low affinity variants of membrane anchored IL2 and/or IL15 have the advantage of providing survival signal selectively to cells in which the variants are expressed and not to stimulate IL2 and/or IL15 signaling in neighboring cells. Therefore, the low affinity variants of membrane anchored IL2 and/or IL15 have the advantage of improved therapeutic index.

A technical challenge with the next generation SAR constructs is the lack of an easy method for their detection, isolation, purification or depletion. The addition of the epitope tags on the SAR constructs have been described but suffer from the problems of interfering with the SAR binding to its target antigen and/or off-target signaling. The applicant has discovered that epitope tags can be added to membrane anchored forms of cytokines (e.g., IL2 and 1L15) without interfering with their binding and signaling activity. The disclosure provides membrane anchored form of IL2 and IL15 and their low affinity variants the also comprise one or more epitope tags (e.g., cetuximab mimotope, rituximab tag, Herceptin mimotope, MYC tag, StreptagII etc.) that can be used for detection, isolation, purification and or depletion of the cells expressing them, including SAR expressing cells. Exemplary epitope tags are known in the literature including Table 37 of PCT/US2021/022641, which is incorporated in its entirety by reference herein. These epitope tags can be used for detection, isolation, purification and or depletion of the immune cells (e.g., SAR-expressing NK or T cells) using the methods described in PCT/US2021/022641.

Increasing efficacy of adoptive immunotherapy has been associated with reports of serious short-term and long-term adverse events, such as CRS, neurotoxicity, graft-versus host disease (GvHD), lymphoproliferation and insertional mutagenesis. Since engineered T-cells can expand and persist for years after administration, it is desirable to include a safety mechanism to allow selective deletion of adoptively infused T-cells in the face of toxicity. Suicide genes enable selective deletion of transduced cells in vivo. Two suicide genes are under clinical testing: HSV-TK and iCasp9.

In order to maximize efficiency of adoptive cell therapy, it is desirable to have a mechanism for monitoring transduction efficiency and selecting transduced cells. A purified population of transduced cells may then be given to the patient.

Some T-cell engineering strategies, including expression of next generation CAR constructs (e.g., SIR, zSIR, Ab-TCR etc.) may not result in transgenic expression of readily detectable surface proteins. In these cases, measurement of transduction and tracking of cells in peripheral blood is difficult. Further, in some settings, it is essential to administer only transduced T-cells, for instance in GvHD gene-therapy protocols. Here, a marker which allows clinical grade sorting is required.

Several marker genes have been described, such as puromycin resistance gene (PAC), tEGFR, CD20, and low-affinity Nerve Growth Factor receptor. More recently, truncated CD34 and RQR8 have been used as marker. These have the advantage that CD34 Miltenyi CliniMACS selection system is readily available for clinical grade sorting.

The marker and suicide genes (e.g., HSV-TK, iCaspase 9, tEGFR etc.) have a long coding sequence and inclusion of these proteins as a marker gene is likely to tax vector packaging capacity and transcriptional efficiency. Finally, none of the above-described suicide and/or marker genes can provide survival signal to the immune cells. Although the suicide, marker and survival functions can be provided by inclusion of two or more genes, this is likely to further tax vector packaging capacity and transcriptional efficiency. There is thus a need for a multi-purpose gene switch which can provide suicide, survival and marker functions.

The disclosure provides multi-purpose gene switches that serve suicide, survival and marker functions. In an exemplary embodiment, a multipurpose switch serves as a life-death (or survival-suicide) switch for the purpose of adoptive cell therapy when ectopically expressed in a cell. In an embodiment, the multipurpose switch has the following formula: SP-D1-L1-D2-L2-D3-L3-D4; where SP is an optional signal peptide that allows cell surface transport of the multipurpose switch and is cleaved to yield the mature peptide, D1 is receptor binding domain which binds to a receptor that promotes cell survival, D2 is a marker/suicide domain, D3 is a hinge domain/stalk domain that allows the D1 and D2 domains to be projected away from the surface of the target cell, D4 is a membrane associating domain (e.g., a transmembrane domain or a membrane anchoring domain) that anchors the molecular switch to the cell membrane and L1, L2 and L3 are optional linker domains.

In an embodiment, the multipurpose switch comprises an in-frame fusion of a first module (D1) comprising a receptor-binding domain to a second module (D2) that serves as a marker/suicide-switch and a third module (D3) that serves as a hinge/stalk domain and a fourth module (D4) that serves as a membrane associating domain. In an embodiment, the D2, D3 and D4 modules are derived from the same endogenous protein. In an embodiment, the D2, D3 and D4 module are derived from different endogenous proteins. In an embodiment, D3 and D4 are derived from the same endogenous protein. In an embodiment, D3 and D4 are derived from the different endogenous proteins.

In an embodiment, the first module (D1) binds to a receptor that is expressed on cell surface, i.e., it binds to the extracellular domain of a receptor. In an embodiment, the first module (D1) binds to a receptor which when bound transmits a pro-survival and/or proliferative signal to the cell. In an embodiment, the first module binds to the receptor in cis (i.e., bind to the receptor expressed on the same cell as the cell expressing the molecular switch). In an embodiment, the first module binds to the receptor in trans (i.e., bind to receptor expressed on a cell other than the cell expressing the molecular switch). In an embodiment, the first module binds to the receptor in cis and in trans. In an embodiment, the first module (D1) comprises the receptor binding domain of a cytokine, a chemokine, a ligand, or a variant or a fragment thereof. Exemplary cytokines, chemokines and ligands include, but are not limited to, one of the following: IL-la, IL-10, IL-2, IL-3, IL-4, IL-5, IL-6, IL-7, IL-8, IL-9, IL-10, IL-11, IL-12, IL-13, IL-14, IL-15, IL-16, IL18, IL-19, IL20, IL-21, IL-22, IL-23, IL27, IL-28, CD40L, 4-1BBL, CD30L, OX40L, FLT3-L, APRIL, BAFF, Rantes, MIP, Erythropoietin, Thrombopoietin, SCF (stem cell factor), G-CSF, GM-CSF and M-CSF etc. In an embodiment, the first module is an antibody, an antibody fragment (e.g., scFv, vL, vH, Fab etc.), a single domain antibody (e.g., vHH, FHVH etc.) or a non-immunoglobulin antigen binding module that can bind to a receptor. In exemplary embodiments, the receptor is selected from one of the following: IL-1R, IL2R, IL-3R, IL-4R, IL-5R, IL-6R, IL-7R, IL-8R, IL-9R, IL-10R, IL-11R, IL-12R, IL-13R, IL-15R, IL-18R, IL-19R, IL-20R, IL-21R, IL-22R, IL-23R, IL-27R, IL-28R, CCR1, CCR3, CCR5, MIP-1R, PF4 receptor, Erythropoeitin-Receptor (Epo-R), TPO-R/MPL, GSF-R, c-Kit, and M-CSF receptor.

In an embodiment, the second module (D2) comprises of the extracellular domain of an endogenous protein or a fragment thereof. In an exemplary embodiment, the D2 comprises the extracellular domain of one or more of the following endogenous proteins or a fragment thereof: CD5; CD19; CD123; CD22; CD30; CD3ε, CD52, CD171; CS1 (SLAMF7, CD319); C-type lectin-like molecule-1 (CLL-1 or CLECL1); CD33; epidermal growth factor receptor variant III (EGFRviii); ganglioside G2 (GD2); ganglioside GD3; BCMA; Tn antigen (Tn Ag); prostate-specific membrane antigen (PSMA); Receptor tyrosine kinase-like orphan receptor 1 (ROR1); Fins Like Tyrosine Kinase 3 (FLT3); Tumor-associated glycoprotein 72 (TAG72); CD38; CD44v6; Carcinoembryonic antigen (CEA); Epithelial cell adhesion molecule (EPCAM); B7H3 (CD276); KIT (CD 117); Interleukin-13 receptor subunit alpha-2 (IL-13Ra2 or CD213A2); Mesothelin; Interleukin 11 receptor alpha (IL-llRa); prostate stem cell antigen (PSCA); Protease Serine 21 (Testisin or PRSS21); vascular endothelial growth factor receptor 2 (VEGFR2); Lewis(Y) antigen; CD24; Platelet-derived growth factor receptor beta (PDGFR-beta); Stage-specific embryonic antigen-4 (SSEA-4); CD20; Folate receptor alpha (FRa or FR1); Folate receptor beta (FRb); Receptor tyrosine-protein kinase ERBB2 (Her2/neu); Mucin 1, cell surface associated (MUC1); epidermal growth factor receptor (EGFR); neural cell adhesion molecule (NCAM); Ephrin B2; fibroblast activation protein alpha (FAP); insulin-like growth factor 1 receptor (IGF-I receptor), carbonic anhydrase IX (CAlX); ephrin type-A receptor 2 (EphA2); sialyl Lewis adhesion molecule (sLe); ganglioside GM3; high molecular weight-melanoma associated antigen (HMWMAA); o-acetyl-GD2 ganglioside (OAcGD2); tumor endothelial marker 1 (TEM1/CD248); tumor endothelial marker 7-related (TEM7R); claudin 6 (CLDN6); thyroid stimulating hormone receptor (TSHR); G protein coupled receptor class C group 5, member D (GPRC5D); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Androgen receptor; Squamous Cell Carcinoma Antigen Recognized By T Cells 3 (SART3); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); and immunoglobulin lambda-like polypeptide 1 (IGLLl), MPL, CD34, LAMP1 TROP2, GFRalpha4, CDH17, CDH6, NYBRI, CDH19, CD200R, Slea (CA19.9; Sialyl Lewis Antigen); Fucosyl-GM1, PTK7, gpNMB, CDH1/CD324, DLL3, CD276/B7H3, IL-2R, IL-4R, IL-6R, IL1lRa, IL13Ra2, IL-17R, CD179b-IGLll, TCRgamma-delta, NKG2D, CD32 (FCGR2A), Tim1-/HVCR1, CSF2RA (GM-CSFR-alpha), TGFbetaR2, Lews Ag, TCR-beta1 chain, TCR-beta2 chain, TCR-gamma chain, TCR-delta chain, FITC, Leutenizing hormone receptor (LHR), Follicle stimulating hormone receptor (FSHR), Gonadotropin Hormone receptor (CGHR or GR), CCR4, SLAMF6, SLAMF4, CD99, Ras G12V, Tissue Factor 1 (TF1), GPRC5D, Claudin18.2 (CLD18A2 or CLDN18A.2), P-glycoprotein, STEAPI, Liv1, Nectin-4, Cripto, gpA33, BST1/CD157, low conductance chloride channel (LCCC), TAJ/TROY, MPL (TPO-R), KIR3DL2, CD32b, CD229, Toso, PD-1, PD-L1, PD-L2, TNFRI, TRAIL-Ri (DR4), TRAIL-R2 (DR5), CTLA4, IL-36R, CD25, LAG3, VEGF-A, MASP-2, Thymic stromal lymphopoietin, Tissue Factor, IFNARI, IL5, IL-6, IL-12, IL-23, IL-17A, IL-13, Angiopoietin-like 3, CGRP, IL-23p19, vWF, C5, IFNγ, CD4, CD8, CD7, NKp30, NKp44, NKp46, NKG2D, PDGRFα, α4β7 integrin, α4 integrin, VEGF, GPIIb/IIIa PCSK9, Blys, and BAFF-R.

In an embodiment, the second module (D2) can be used to induce death of the cells expressing the molecular switch. In an embodiment, the second module (D2) can be used to induce death of the cells expressing the molecular switch when bound by an agent. In an exemplary embodiment, the agent that induces death of cells expressing the molecular switch when bound to the second module is an antibody, an antibody fragment, an scFv, a single domain antibody, a non-immunoglobulin antigen binding domain, an antibody drug conjugate, a bispecific antibody or a fragment thereof or a cell (e.g., CAR-T, SAR-T, SAR-NK cell etc.). In an embodiment, the second module can be used to selectively enrich or deplete cells expressing the molecular switch. In an embodiment, the second module can be used to selectively detect, enrich and/or deplete cells expressing the molecular switch when bound by an agent. In an exemplary embodiment, the agent that can be used to selectively detect, enrich and/or deplete the cells expressing the molecular switch when bound to the second module is an antibody, a single domain antibody, a non-immunoglobulin antigen binding domain or a fragment thereof. In an embodiment, the molecular switch is used to selectively detect, enrich and/or deplete cells ex vivo. In an embodiment, the molecular switch is used to selectively deplete cells in vivo. In an embodiment, the agent (i.e., an antibody, antibody drug conjugate, bispecific antibody, a non-immunoglobulin antigen binding domain or a fragment thereof) that is used to detect, deplete or enrich cells expressing the molecular switch has been approved for human administration by the FDA. Exemplary agents that have been approved by FDA or are pending FDA approval for human administration are known in the art and include, but are not limited to, Rituximab, Herceptin, Erbitrux, Adcetris, Enbrel, Tremelimumab, Mosunetuzumab, Teclistamab, Donanemab, Spesolimab, Faricimab, Tislelizumab, belantamab mafodotin, Pembrolizumab, nivolumab and Qbend etc. Methods to selectively deplete and/or enrich cells using antibodies are known in the art (e.g., WO 2018/178378). In an embodiment, the agent that is used to detect, deplete or enrich cells expressing the molecular switch is approved by the FDA for ex vivo clinical use. An exemplary such agent is an antibody against CD34 that has been approved by the FDA to be used in conjunction with the clinically approved CliniMACS CD34 system (Miltenyi).

The molecular switch of the disclosure comprises a hinge/stalk sequence (D3) which, when the polypeptide is expressed at the surface of a target cell, causes the D1 and D2 domains to be projected away from the surface of the target cell.

The stalk sequence (D3) causes the D1 and D2 to be sufficiently distanced from the cell surface to facilitate binding of, for example, of the D1 to the receptor and/or an antibody to the D2 domain.

The stalk sequence (D3) elevates the D1-D2 domains from the cell surface.

The stalk sequence may be a substantially linear amino acid sequence. The stalk sequence may be sufficiently long to distance the D1 and D2 domains from the surface of the target cell but not so long that its encoding sequence compromises vector packaging and transduction efficiency. The stalk sequence may, for example be between 20 and 100 amino acids in length. The stalk sequence may be approximately 40-50 amino acids in length.

The stalk sequence may be highly glycosylated.

The stalk sequence may comprise or be approximately equivalent in length to the sequence represented by SEQ ID NO (DNA):7132 and SEQ ID NO (PRT):7824.

The stalk/hinge sequence is operationally linked to a transmembrane domain (D4), optionally together with an intracellular anchor sequence. The transmembrane domain and intracellular anchor sequence may be derived from the same protein as extracellular part of the stalk sequence or it/they may be derived from a different protein. The stalk/hinge and transmembrane domains and intracellular anchor sequence may be derivable from CD8. An exemplary CD8 hinge and transmembrane sequence is provide in SEQ ID NO (PRT):3603.

The molecular switch may comprise L1, L2 and L3 are optional linker (or spacer) domains, which may be the same or different. Exemplary linkers are provided in SEQ ID NO: 3418-3434. The molecular switch may also comprise a signal peptide at the amino terminus. Exemplary signal peptides are provided in SEQ ID NO: 2425-2428. Once the polypeptide is expressed by the target cell, the signal peptide is cleaved, resulting in the mature peptide product.

An exemplary multipurpose switch is Synth-IL2-Nde-tBCMA-L244ter (SEQ ID NO (DNA):7152 and SEQ ID NO (PRT): 7843) and comprises the IL2 receptor binding domain of IL2 fused in frame to the extracellular-domain and the transmembrane domain of BCMA. This multipurpose switch when expressed in immune cells (e.g., T cells or NK cells etc.) provides them with a survival signal by binding to the IL2 receptor through the N-terminal D1 module comprising the IL-2R binding domain of IL2. The second module (D2) of this multipurpose switch comprises the extracellular domain of BCMA which is recognized by BCMA-binding agents (e.g., BCMA antibodies) and can be used for the detection, selective depletion and/or enrichment of transgene (e.g., SAR) expressing cells. The extracellular domain of BCMA comprising the second (D2) module can be also used for selective suicide of transgene (e.g., SAR) expressing cells by the use of BCMA-targeted agents, such as an antibody or an antibody drug-conjugates targeting BCMA (e.g., belantamab mafodotin). The third (D3) and the fourth (D4) modules in this molecular switch comprises of the hinge/stalk and transmembrane domains of BCMA and serves to anchor the switch to the cell membrane.

As the molecular switches are modular in format, the different modules can be replaced by other modules as long as the resulting switch retains at least one of its biological activities (e.g., ability to serve as a survival switch or a suicide switch etc.). Thus, the IL2 module can be replaced by a different cytokine (e.g., IL15, IL18, IL21 etc.) These multipurpose proteins provide a pro-survival signal through their cytokine moiety (e.g., IL2, IL15, IL18, IL21 etc.) but can be used to kill-off the cells by the use of an agent (e.g., an antibody) that binds to the second module (e.g., RQR3, tBCMA, tHer2, tEGFR, tCD19, tCD30 etc.), thereby acting as a suicide gene that allow selective deletion of administered T cells in the face of toxicity. The second module (e.g., RQR3, tBCMA, tHer2, tEGFR, tCD19, tCD30 etc.) can be also used as a marker for measurement of transduction and to allow selection of transduced cells.

In an exemplary embodiment, the IL2 module can be replaced by low-affinity IL2 variants, IL15 or IL15 variants. Exemplary multipurpose switches comprising low-affinity IL2 variants fused to tBCMA are provided in SEQ ID NO (DNA):7152-7155 and SEQ ID NO (PRT): 7844-7847. Exemplary multipurpose switches comprising IL-15 and its low affinity variant fused to tBCMA are provided in SEQ ID NO (DNA):7156-7157 and SEQ ID NO (PRT): 7848-7849. In another exemplary embodiment, the tBCMA module can be replaced by other modules. An exemplary multipurpose switch comprises IL2 fused to tCD30 is provided in SEQ ID NO (DNA):7158 and SEQ ID NO (PRT): 7850. Exemplary multipurpose switches comprising cytokines fused to tEGFR and other surface proteins (e.g., Her3, CD19, PD1, PDL1, CD40 etc.) can be constructed similarly and used in alternate embodiments of the disclosure.

The multipurpose switch expression cassettes are compact in size and can be easily packaged in viral vectors. They are much more manageable size than the expression cassettes encoding individual marker, suicide and survival genes, which would require separate promoters. They have the added advantage of comprising survival, suicide and marker gene elements with sensitivity at least equal to that demonstrated by the individual genes.

In an embodiment, the second module (D2) is a synthetic module comprising one or more copies of an epitope or a mimotope. In an embodiment, the epitope is present in the extracellular domain of an endogenous protein. In an embodiment, the mimotope mimics an epitope that is present in the extracellular domain of an endogenous protein. Exemplary endogenous proteins are presented in the preceding section. An exemplary synthetic module comprising one or more copies of an epitope or a mimotope is RQR8 (SEQ ID NO: 9619), which is a module harboring a CD34 epitope and two CD20 mimotopes and has been described in WO/2013/153391, which is incorporated in its entirety by reference herein. The RQR8 module allows selection with the clinically approved CliniMACS CD34 system (Miltenyi). Further, the construct binds the widely used pharmaceutical antibody rituximab, resulting in selective deletion of transgene-expressing cells. Additional exemplary multi-purpose molecular switches and include fusion proteins comprising IL2 or its variants and tBCMA (SEQ ID NO (DNA): 7151-7155), IL15 or its variants and tBCMA (SEQ ID NO (DNA):7156-7157), IL2 and its variants and tHer2, IL2 and its variants and tEGFR, IL2 and its RQR8, IL2 and its variants and CD30 (SEQ ID NO: 7850) etc. As the multipurpose switches are modular in format, one module can be replaced with a different module.

The polypeptides encoding the multipurpose switches of the disclosure may comprise or consist of a variant of the sequence shown as SEQ ID No. 7843-7850 and 9621-9625, which has at least 70%, 80% or 90% identity with the sequence shown as SEQ ID No. 7843-7850 and 9621-9625, as long as it retains the functional activity of the SEQ ID No. 7843-7850 and 9621-9625 polypeptide. For example, the variant of sequence 7843-7847 should (i) bind IL-2R; (ii) bind BCMA antibodies (e.g., J6M0 or belantamab mafodotin) (iii) when expressed on the surface of a cell, induce killing of the cell in the presence of J6M0 or belantamab mafodotin. The J6M0 has been described in U.S. Pat. No. 9,273,141B2.

Homology comparisons may be conducted by eye or with the aid of readily available sequence comparison programs, such as the GCG Wisconsin Bestfit package. The multipurpose molecular switches of the disclosure can be in the form of a fusion protein, in which the polypeptide is fused to a protein of interest (POI). The fusion protein may comprise a self-cleaving peptide (e.g., P2A or F2A) between the polypeptide encoding the multipurpose switch and the protein of interest.

The protein of interest is a molecule for expression at the surface of a target cell. The POI may exert a therapeutic or prophylactic effect when the target cell is in vivo. The POI may be a SAR (e.g., a CAR, SIR, zSIR, HIT, STAR, cTCR, Ab-TCR, TFP, TAC, recombinant TCR etc.) or an endogenous TCR.

The disclosure also provides a nucleic acid sequence capable of encoding a multipurpose switch encoding polypeptide or fusion protein of the disclosure.

The nucleic acid, when expressed by a target cell, causes the encoded multipurpose switch polypeptide to be expressed at the cell-surface of the target cell. Where the nucleic acid encodes both the multipurpose switch polypeptide and POI (for example as a fusion protein), it should cause both the polypeptide of the disclosure and the POI to be expressed at the surface of the target cell.

The nucleic acid sequence may be RNA or DNA, such as cDNA.

The disclosure also provides a vector which comprises a nucleic acid sequence of the multipurpose molecular switch. The vector may also comprise a transgene of interest, i.e., a gene encoding a POI (e.g., a SAR).

The vector should be capable of transfecting or transducing a target cell, such that they express the polypeptide encoding the multipurpose switch and optionally a protein of interest.

The vector may be a non-viral vector such as a plasmid. The vector may be a viral vector, such as a retroviral or lentiviral vector. The vector may comprise a nucleic acid encoding the polypeptide and a nucleic acid comprising the POI as separate entities, or as a single nucleotide sequence. If they are present as a single nucleotide sequence they may comprise one or more internal ribosome entry site (IRES) sequences between the two encoding portions to enable the downstream sequence to be translated. In an embodiment, the multipurpose molecular switch and the POI may be expressed from a single vector using separate promoters. In another embodiment, the multipurpose switch and the POI may be expressed from separate vectors.

The disclosure also provides a cell which expresses a multipurpose switch polypeptide of the disclosure. The cell may coexpress the multipurpose switch polypeptide and a POI (e.g., a SAR) at the cell surface. The disclosure also provides a cell which comprises a nucleic acid sequence capable of encoding a multipurpose switch polypeptide of the disclosure. The cell may have been transduced or transfected with a vector according to the disclosure. The cell may be suitable for adoptive cell therapy. The cell may be a T cell, such as a cytotoxic T lymphocyte (CTL). The T cell may have an existing specificity. For example, it may be an Epstein-Barr virus (EBV)-specific T cell. The cell may be an NK cell, NKT cell, iPSC-derived T cell or synthetic T cell. The cell may be derived from a patient. For example, the cell may have been removed from a patient and then transduced ex vivo with a vector according to the disclosure. T cell populations which are suitable for ACT include: bulk peripheral blood mononuclear cells (PBMCs), CD8+ cells (for example, CD4-depleted PBMCs); PBMCs that are selectively depleted of T-regulatory cells (Tregs); isolated central memory (Tcm) cells; EBV-specific CTLs; and ti-virus-specific CTLs.

The disclosure also comprises a cell population which comprises a cell according to the disclosure. The cell population may have been transduced with a vector according to the disclosure. A proportion of the cells of the cell population may express a multipurpose switch polypeptide according to the disclosure at the cell surface. A proportion of the cells of the cell population may co-express a multipurpose switch polypeptide and a POI (e.g., SAR) at the cell surface. The cell population may be ex vivo patient-derived cell population.

The disclosure provides a method for measuring transduction with a transgene of interest (which encodes a protein of interest POI, e.g., a SAR), which comprises the step of transducing a population of cells with a vector which coexpresses the multipurpose switch polypeptide of the disclosure and the protein of interest (e.g., a SAR) and detecting expression of the multipurpose switch (e.g., BCMA, QBEnd10-binding epitope etc.) on the surface of cells, wherein the proportion of cells expressing the multipurpose switch polypeptide of the disclosure corresponds to the proportion of cells transduced with the transgene of interest.

The disclosure also provides a method for selecting cells expressing a POI (e.g., a SAR) which comprises the following steps:
(i) detecting expression of the multipurpose switch (e.g., BCMA-, Her2-, or QBEnd10-binding epitope) on the surface of cells transfected or transduced with a vector of the disclosure which comprises a nucleotide sequence encoding the POI (e.g., a SAR); and
(ii) selecting cells which are identified as expressing the multipurpose switch (e.g., BCMA-, Her2-, or QBEnd10-binding epitope).

Cells may be identified and/or sorted by methods known in the art such as FACS or Miltenyi cliniMACS system.

The disclosure also provides a method for preparing a purified population of cells enriched for cells expressing a POI (e.g., SAR) which comprises the step of selecting cells expressing a POI (e.g., a SAR) from a population of cells using the method described above. The disclosure also provides a purified population of POI-expressing cells prepared by such a method. In the purified population of cells, at least 80%, 85%, 90% or 95% of the cells may express a POI (and a multipurpose switch polypeptide according to the disclosure).

The disclosure also provides a method for tracking transduced cells in vivo which comprises the step of detection of expression of the polypeptide of the disclosure at the cell surface. Cells may be tracked in vivo by methods known in the art such as bioluminescence imaging. For such applications, the polypeptide of the disclosure may be engineered to be co-expressed with a detectable protein, such as luciferase.

The disclosure also provides a method for deleting cells transduced by a vector according to the disclosure, which comprises the step of exposing the cells to an agent that binds to the multipurpose switch polypeptide. In an embodiment, the agent binds to the D2 domain of the multipurpose switch polypeptide. In an embodiment, the agent is an antibody (e.g., rituximab, ertibtrux or J6M0) and cells are exposed to the antibody in the presence of complement. In an embodiment, the agent is an antibody drug conjugate (e.g., belantamab mafodotin, T-DM1 or Enhertu etc.). In an embodiment, the multipurpose switch comprises tBCMA or the variants or fragments thereof and the agent is J6M0 or belantamab mafodotin. In an embodiment, the multipurpose switch comprises tHer2 or the variants or fragments thereof and the agent is Herceptin, T-DMlor Enhertu. In an embodiment, the multipurpose switch comprises RQR8 or the variants or fragments thereof and the agent is rituximab. In an embodiment, the multipurpose switch comprises tEGFR or the variants or fragments thereof and the agent is Erbitrux.

When the multipurpose switch polypeptide of the disclosure is expressed at the surface of a cell, binding of the agent (e.g., rituximab) to the D2 domain of the polypeptide causes lysis of the cell. More than one molecule of agent (e.g., Rituximab) may bind per multipurpose switch polypeptide expressed at the cell surface.

Deletion of cells may occur in vivo, for example by administering the agent (e.g., Rituximab, Herceptin, Erbitrux, J6M0, belantamab mafodotin, T-DM1 or Enhertu etc.) to a patient. The decision to delete the transferred cells may arise from undesirable effects being detected in the patient which are attributable to the transferred cells. For example, unacceptable levels of toxicity may be detected. The dose, route and frequency of administration of the different agent is known in the art and will vary depending on the agent and clinical condition of the subject. In an embodiment, more than one dose of the agent is administered.

Adoptive transfer of genetically modified T cells is an attractive approach for generating desirable immune responses, such as an anti-tumor immune response. The disclosure provides a method for treating and/or preventing a disease in a subject, which comprises the step of administering a cell according to the disclosure to the subject. The method may comprise the step of administering a population of cells to a subject. The population of cells may be enriched for cells expressing a transgene of interest using a method described above.

The method may involve the following steps:
(i) taking a sample of cells, such as a blood sample from a patient,
(ii) extracting the T-cells,
(iii) transducing or transfecting the T cells with a vector of the disclosure which comprises a nucleic acid sequence encoding the multipurpose switch and a transgene (e.g., SAR) of interest,
(iv) expanding the transduced cells ex-vivo
(v) returning the cells to the patient.
   The transduced cells may possess a desired therapeutic property such as enhanced tumor specific targeting and killing.

A technical challenge with the next generation SAR constructs (e.g., SIR, zSIR, Ab-TCR, HIT, STAR etc.) is the lack of an easy method for their detection, isolation, purification or depletion. The addition of the kill switches on the CAR constructs have been described but suffer from the problems of interfering with the SAR binding to its target antigen and/or off-target signaling. The applicant has discovered that cytokines (e.g., IL2 and 1L15 etc.) can be fused in frame to the membrane anchored form of a molecule (e.g., BCMA, CD30 etc.) without interfering with their binding and signaling activity. The disclosure provides cytokines (e.g., IL2 and IL15 etc.) and their low affinity variants fused to a membrane anchored molecule (e.g., BCMA or CD30). As there are FDA approved antibodies and antibody drug conjugates available against BCMA and CD30 (e.g. Adcetris), these molecules can be used as kill switches to eradicate cells (e.g., SAR-cells) expressing them in case of toxicity.

The fusion construct may also comprise one or more epitope tags (e.g., cetuximab mimotope, rituximab tag, Herceptin mimotope, MYC tag, StreptagII etc.) that can be used for detection, isolation, purification and or depletion of the cells expressing them, including SAR expressing cells. Exemplary epitope tags are known in the literature (e.g., SEQ ID NO: 3423-3434. These epitope tags can be used for detection, isolation, purification and or depletion of the immune cells (e.g., SAR-expressing NK or T cells) using the methods described in PCT/US2021/022641.

The article of manufacture can comprise a container and a label or package insert on or associated with the container. Suitable containers include, for example, bottles, vials, syringes, etc. The containers may be formed from a variety of materials such as glass or plastic. Generally, the container holds a composition which is effective for treating a disease or disorder described herein, and may have a sterile access port (for example the container may be an intravenous solution bag or a vial having a stopper pierceable by a hypodermic injection needle). At least one active agent in the composition is an effector cell presenting on its surface an SAR of the disclosure. The label or package insert indicates that the composition is used for treating the particular condition. The label or package insert will further comprise instructions for administering the SAR effector cell composition to the patient. Articles of manufacture and kits comprising combinatorial therapies described herein are also contemplated.

Package insert refers to instructions customarily included in commercial packages of therapeutic products that contain information about the indications, usage, dosage, administration, contraindications and/or warnings concerning the use of such therapeutic products. In some embodiments, the package insert indicates that the composition is used for treating a target antigen-positive cancer (such as adrenocortical carcinoma, bladder cancer, breast cancer, cervical cancer, cholangiocarcinoma, colorectal cancers, esophageal cancer, glioblastoma, glioma, hepatocellular carcinoma, head and neck cancer, kidney cancer, lung cancer, melanoma, mesothelioma, multiple myeloma, pancreatic cancer, pheochromocytoma, plasmacytoma, neuroblastoma, ovarian cancer, prostate cancer, sarcoma, stomach cancer, uterine cancer or thyroid cancer). In other embodiments, the package insert indicates that the composition is used for treating a target antigen-positive viral infection (for example infection by CMV, EBV, HCV etc.).

Additionally, the article of manufacture may further comprise a second container comprising a pharmaceutically acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

Kits are also provided that are useful for various purposes, e.g., for treatment of a target antigen-positive disease or disorder described herein, optionally in combination with the articles of manufacture. Kits of the disclosure include one or more containers comprising an SAR effector cell composition (or unit dosage form and/or article of manufacture), and in some embodiments, further comprise another agent (such as the agents described herein) and/or instructions for use in accordance with any of the methods described herein. The kit may further comprise a description of selection of individuals suitable for treatment.

Instructions supplied in the kits of the disclosure are typically written instructions on a label or package insert (e.g., a paper sheet included in the kit), but machine-readable instructions (e.g., instructions carried on a magnetic or optical storage disk) are also acceptable.

For example, in some embodiments, the kit comprises a composition comprising an effector cell presenting on its surface an SAR. In some embodiments, the kit comprises a) a composition comprising an effector cell presenting on its surface an SAR, and b) an effective amount of at least one other agent, wherein the other agent increases the expression of MHC proteins and/or enhances the surface presentation of peptides by MHC proteins (e.g., IFNy, IFNP, IFNa, or Hsp90 inhibitor). In some embodiments, the kit comprises a) a composition comprising an effector cell presenting on its surface an SAR, and b) instructions for administering the SAR effector cell composition to an individual for treatment of a target antigen-positive disease (such as cancer or viral infection). In some embodiments, the kit comprises a) a composition comprising an effector cell presenting on its surface an SAR, b) an effective amount of at least one other agent, wherein the other agent increases the expression of MHC proteins and/or enhances the surface presentation of peptides by MHC proteins, and c) instructions for administering the SAR effector cell composition and the other agent(s) to an individual for treatment of a target antigen-positive disease (such as cancer or viral infection). The SAR effector cell composition and the other agent(s) can be present in separate containers or in a single container. For example, the kit may comprise one distinct composition or two or more compositions wherein one composition comprises the SAR effector cell and another composition comprises the other agent.

In some embodiments, the kit comprises a) a composition comprising an SAR, and b) instructions for combining the SAR with effector cells (such as effector cells, e.g., T cells or natural killer cells, derived from an individual) to form a composition comprising the effector cells presenting on their surface the SAR and administering the SAR effector cell composition to the individual for treatment of a target antigen-positive disease (such as cancer or viral infection). In some embodiments, the kit comprises a) a composition comprising an SAR, and b) an effector cell (such as a cytotoxic cell).

In some embodiments, the kit comprises a) a composition comprising an SAR, b) an effector cell (such as a cytotoxic cell), and c) instructions for combining the SAR with the effector cell to form a composition comprising the effector cell presenting on its surface the SAR and administering the SAR effector cell composition to an individual for the treatment of a target antigen-positive disease (such as cancer or viral infection).

In some embodiments, the kit comprises a nucleic acid (or set of nucleic acids) encoding an SAR. In some embodiments, the kit comprises a) a nucleic acid (or set of nucleic acids) encoding an SAR, and b) a host cell (such as an effector cell) for expressing the nucleic acid (or set of nucleic acids). In some embodiments, the kit comprises a) a nucleic acid (or set of nucleic acids) encoding an SAR, and b) instructions for i) expressing the SAR in a host cell (such as an effector cell, e.g., a T cell), ii) preparing a composition comprising the host cell expressing the SAR, and iii) administering the composition comprising the host cell expressing the SAR to an individual for the treatment of a target antigen-positive disease (such as cancer or viral infection). In some embodiments, the host cell is derived from the individual. In some embodiments, the kit comprises a) a nucleic acid (or set of nucleic acids) encoding an SAR, b) a host cell (such as an effector cell) for expressing the nucleic acid (or set of nucleic acids), and c) instructions for i) expressing the SAR in the host cell, ii) preparing a composition comprising the host cell expressing the SAR, and iii) administering the composition comprising the host cell expressing the SAR to an individual for the treatment of a target antigen-positive disease (such as cancer or viral infection).

The kits of the disclosure are in suitable packaging. Suitable packaging includes, but is not limited to, vials, bottles, jars, flexible packaging (e.g., sealed Mylar or plastic bags), and the like. Kits may optionally provide additional components such as buffers and interpretative information. The present application thus also provides articles of manufacture, which include vials (such as sealed vials), bottles, jars, flexible packaging, and the like.

The instructions relating to the use of the SAR effector cell compositions generally include information as to dosage, dosing schedule, and route of administration for the intended treatment. The containers may be unit doses, bulk packages (e.g., multi-dose packages) or sub-unit doses. For example, kits may be provided that contain sufficient dosages of an SAR effector cell composition as disclosed herein to provide effective treatment of an individual for an extended period, such as any of a week, 8 days, 9 days, 10 days, 11 days, 12 days, 13 days, 2 weeks, 3 weeks, 4 weeks, 6 weeks, 8 weeks, 3 months, 4 months, 5 months, 7 months, 8 months, 9 months, or more. Kits may also include multiple unit doses of the SAR and pharmaceutical compositions and instructions for use and packaged in quantities sufficient for storage and use in pharmacies, for example, hospital pharmacies and compounding pharmacies.

The disclosure provides vL, vH and scFv targeting a membrane exposed epitope of Heat shock protein 70 (Hsp70) (Table 47). The disclosure also provides SAR targeting Hsp70 based on these vL, vH and scFv polypeptides targeting Hsp70. The SEQ ID of exemplary SARs targeting Hsp70 are provided in (SEQ ID NO (DNA): 7754-7808 and SEQ ID NO (PRT): 8446-8500). The SARs are expressed in immune cells (e.g., T cells and NK cells etc.) using lentiviral mediated gene transfer and tested for cytotoxicity against Hps70 expressing Daudi cells using in vitro and in vivo assays described in this disclosure.

In vivo delivery of SARs with a number of different backbones is tested in a patient with Acute lymphocytic leukemia. A number of delivery methods are tried, including lentiviral mediated gene transfer and gene transfer with viral like particles. It is observed that SARs with the backbone of a SIR, a cTCR and Ab-TCR are safer for in vivo gene delivery as compared with the SAR with the backbone of a 1^{st} generation CAR, a 2^{nd} generation CAR, a 3^{rd} generation CAR, a TFPε, TFPγ, TFPδ, TFPƒ, CD16-SAR, NKp30-SAR, NKp44-SAR, NKp46-SAR, DAP10-SAR, or NKG2D SAR. It is observed that the lentiviral vectors encoding a CD19-SAR with the backbone of a SIR, acTCR and Ab-TCR insert less frequently in circulating CD19-expressing B-ALL cells as compared to the lentiviral vectors encoding a SAR with the backbone of a 1^{st} generation CAR, a 2^{nd} generation CAR, a 3^{rd} generation CAR, a TFPε, TFPγ, TFPδ, TFPƒ, CD16-SAR, NKp30-SAR, NKp44-SAR, NKp46-SAR, DAP10-SAR, or NKG2D SAR. It is further observed that even upon insertion, SARs with the backbone of a SIR, acTCR and Ab-TCR do not express in circulating CD19-expressing B-ALL cells while good expression of SAR with the backbone of a 1” generation CAR, a 2^{nd} generation CAR, a 3rd generation CAR, a TFPε, TFPγ, TFPδ, TFPƒ, CD16-SAR, NKp30-SAR, NKp44-SAR, NKp46-SAR, DAP10-SAR, or NKG2D SAR is observed in circulating B-ALL cells. It is further noted that the risk of relapse of B-ALL due to accidental insertion of the SAR is lower with the SARs with the backbones of SIR, a cTCR and Ab-TCR as compared with the SAR with the backbone of a 1” generation CAR, a 2^{nd} generation CAR, a 3^{rd} generation CAR, a TFPε, TFPγ, TFPδ, TFPƒ, CD16-SAR, NKp30-SAR, NKp44-SAR, NKp46-SAR, DAP10-SAR, or NKG2D SAR.

It has been observed that CAR-T cells targeting antigens (e.g., mesothelin, PSMA etc.) expressed on solid tumor show poor expansion upon infusion into the patient. The current disclosure provides a solution to this problem by providing SARs that can target antigens that are expressed on blood cells (e.g., CD19, BCMA, CD20 etc.). Such SARs can be co-expressed in the immune cells along with SARs targeting solid tumors to provide proliferative advantage to the SAR-T cells targeting the solid tumor. For example, a SAR targeting CD19 (SEQ ID NO: 7660) can be co-expressed in immune cells (e.g., T cells or NK cells) with a SAR targeting PSMA (SEQ ID NO: 7617) or a SAR targeting Her2. It is observed that the immune cells (e.g., T cells or NK cells) expressing PSMA SAR and also co-expressing the CD19 SAR show greater proliferation and in vivo persistence as compared to immune cells expressing the PSMA SAR alone. A unique advantage of the SAR of the current disclosure for generating bispecific or multi-specific immune cells (e.g., bispecific or multispecific CAR-T or CAR-NK cells) is based on their relatively small size as compared to the 1” generation or the 2^{nd} generation CARs. Therefore, they can be easily packaged in the viral vectors (e.g., lentiviral or retroviral vectors). In addition, the SARs of the current disclosure comprise many different signaling chains (e.g., CD16, NKp30, NKp44, NKp46, DAP10, NKG2D, CD3z etc.). The SARs with different signaling chains (i.e., backbones) can be combined so as to generate a variety of bispecific and multispecific SARs that do not compete with each other for signaling proteins. Thus, the SARs of the current disclosure can be used to generate a diverse immune response.

As stated previously, there is a packaging limit to the size of the lentivirus inserts. The disclosure describes the use of internal ribosomal entry sequence (IRES) from human herpes virus 8 (Kaposi's sarcoma associated herpes virus or KSHV) for expression of a gene for cell therapy and other genetic engineering applications. As compared to other known IRES, the KSHV IRES (SEQ ID NO: 7116) is relatively small in size and therefore can be easily packaged in viral vectors. We generated a lentiviral vector encoding a double chain SIR in which tBCMA was expressed after the SIR cassette using an intervening KSHV IRES sequence. We observed effective expression of tBCMA in T cells infected with the lentiviral vector. We also observed good titer of the resulting lentiviral vector when packaged in 293FT cells using standard packaging mix. We also observed high level expression of the SIR in the T cells, suggesting that presence of KSVH IRES does not impact the expression of the upstream SIR. Both the expression of the SIR and tBCMA in the T cells infected with the above construct compared favorably to their expression in T cells infected with a lentiviral vector in which the tBCMA was expressed using a F2A ribosomal skip sequence.

The disclosure contemplates the following exemplary inventive aspects:

Aspect 1. A synthetic antigen receptor (SAR) that specifically binds to a target antigen, the SAR comprising: (i) a first module comprising one or more heterologous antigen binding domains selected from the group consisting of: a) an antibody; b) an antibody fragment; c) a heavy chain variable region of an antibody (vH domain) or a fragment thereof, d) a light chain variable region of an antibody (vL domain) or a fragment thereof, e) a single chain variable fragment (scFv) or a fragment thereof, f) a single domain antibody (SDAB) or a fragment thereof, g) a vHH domain or a fragment thereof, h) a monomeric variable region of an antibody; i) a single vH domain (SVH) or a fragment thereof, j) a single vL domain (SVL) or a fragment thereof, k) a non-immunoglobulin antigen binding scaffold selected from a DARPIN, an affibody, an affilis, an adnectin, an affitin, an obody, an repebody, an fynomer, an alphabody, an avimer, an atrimer, an centyrin, an pronecti, an anticalins, an kunitz domain, an Armadillo repeat protein, a D domain, and a fragment of any of the foregoing; l) a ligand-binding domain of a receptor or a fragment thereof, m) a receptor-binding domain of a ligand; n) a bispecific-antibody, -antibody fragment, -scFV, -vHH, -SDAB, -non-immunoglobulin antigen binding scaffold, -receptor or -ligand; o) an autoantigen or a fragment thereof, p) an adaptor binding domain or a fragment thereof, q) an Fc binding domain or a fragment thereof, r) a TCR or an HLA-independent TCR or a fragment thereof, and s) Va, Vb, Vg or Vd fragment of a TCR or a fragment thereof, (ii) a second module that comprise at least one membrane associated domain, wherein the membrane associated domain can be a transmembrane domain or a membrane anchoring domain; and (iii) an optional third module comprising one or more cytosolic domains, where the first, second, and the optional third modules are operationally linked via one or more optional linkers.

Aspect 2. A single chain SAR of aspect 1, where the first module comprising one or more heterologous antigen binding domains are operationally linked via optional linkers to a polypeptide comprising: (1) the entire or partial extracellular antigen binding domain, optional hinge domain, transmembrane/membrane associated domain and optional cytosolic domain of a naturally occurring receptor or a fragment or variant thereof, or (2) the hinge domain, transmembrane/membrane associated domain and optional cytosolic domain of a naturally occurring receptor or a fragment or variant thereof; or (3) the transmembrane/membrane associated domain and optional cytosolic domain of a naturally occurring receptor or a fragment or variant thereof, or (4) cytosolic domain of a naturally occurring receptor or a fragment or variant thereof; or (5) the entire or partial extracellular domain, the hinge domain, the transmembrane domain and cytosolic domain of a signaling adaptor or a variant or a fragment thereof.

Aspect 3. A SAR of aspect 2, wherein a) the naturally occurring receptor does not comprise a T cell receptor selected from the group consisting of TCRα, TCRβ, TCRγ, TCRδ and preTCRα; and/or b) the naturally occurring receptor does not comprise a T cell receptor module (TCRM); and/or c) the signaling adaptor is not a CD3 adaptor selected from the group of CD3ƒ, CD3γ, CD3E and CD36; and/ord) the signaling adaptor is not FcRγ.

Aspect 4. A SAR of aspects 2 or 3, wherein the naturally occurring receptor is a Type I membrane protein with an N-terminal extracellular domain and the N-terminus of a polypeptide comprising one or more heterologous antigen binding domains is operationally linked via optional linkers to the N-terminus or near the N-terminus of the polypeptide comprising the a) the entire or partial extracellular antigen binding domain, optional hinge domain, transmembrane/membrane associated domain and optional cytosolic domain of the naturally occurring receptor polypeptide chain or a fragment or variant thereof, or b) the hinge domain, transmembrane/membrane associated domain and optional cytosolic domain of the naturally occurring receptor polypeptide chain or a fragment or variant thereof, or c) the transmembrane/membrane associated domain and optional cytosolic domain of the naturally occurring receptor polypeptide chain or a fragment or variant thereof, or d) cytosolic domain of the naturally occurring receptor polypeptide chain or a fragment or variant thereof.

Aspect 5. A SAR of aspect 4, wherein the naturally occurring receptor Type I membrane protein is selected from the group consisting of CD16A, CD16B, CD64, CD32, NKp30, NKp44, NKp46, KIR2DL1, KIR2DL2, KIR2DL3, KIR2DL5A, KIR2DL5B, KIR3DL1, KIR3DL2, KIR3DL4, KIR2DL4, KIR2DS1, KIR2DS2, KIR2DS3, KIR2DS4, KIR2DS5, KIR3DS1, DNAM-1, 2B4, OX40, CD28, 4-1BB, CD27, CD81, CD2, CD5, TNFR-I, TNFR-II, Fas, CD30, CD40, CRTAM, TIGIT, CD96, SLAMF6, SLAMF7, CD100, CD160, CEACAM, ILT2, LAIR1, variants and fragments thereof.

Aspect 6. A SAR of aspects 2 or 3, wherein the naturally occurring receptor is a Type II membrane protein with a C-terminal extracellular domain and the N-terminus of a polypeptide encoding one or more heterologous antigen binding domains is operationally linked via optional linkers to the C-terminus or near the C-terminus of a polypeptide comprising: a) the entire or partial extracellular antigen binding domain, optional hinge domain, transmembrane/membrane associated domain and optional cytosolic domain of the naturally occurring receptor polypeptide chain or a fragment or variant thereof, or b) the hinge domain, transmembrane/membrane associated domain and optional cytosolic domain of the naturally occurring receptor polypeptide chain or a fragment or variant thereof, or c) the transmembrane/membrane associated domain and optional cytosolic domain of the naturally occurring receptor polypeptide chain or a fragment or variant thereof, or d) cytosolic domain of the naturally occurring receptor polypeptide chain or a fragment or variant thereof.

Aspect 7. A SAR of aspect 6, further comprising the N-terminus of a polypeptide comprising the cytosolic domain of a signaling adaptor operationally linked to the N-terminus of the Type II membrane protein.

Aspect 8. The SAR of aspect 7, wherein the signaling adaptor is selected from the group of CD3ƒ, FcRγ, DAP10 or DAP10.

Aspect 9. The SAR of aspect 8, further comprising the N-terminus of a polypeptide comprising one or more co-stimulatory domains operationally linked to the N-terminus of the cytosolic domain of the signaling adaptor.

Aspect 10. The SAR of aspect 9, wherein the one or more co-stimulatory domains are selected from the group consisting of CD28, 4-1BB, OX40, 2B4, CD27, CD81, CD2, CD5, BAFF-R, CD30, CD40, HVEM and ICOS.

Aspect 11. The SAR of aspect 10, which is co-expressed with an accessory module comprising DAP10.

Aspect 12. A SAR of anyone of aspects 6-10, wherein the naturally occurring receptor Type II membrane protein is selected from the group consisting of NKG2D, NKG2C, NKG2A, NKG2E, NKG2F, KLRG1, CD94, CD161, variants thereof and fragments thereof.

Aspect 13. A SAR of aspect 3, wherein the entire or partial extracellular antigen binding domain, the optional hinge domain, the transmembrane domain and the optional cytosolic domain are all derived from a single naturally occurring receptor and are present in one continuous polypeptide chain.

Aspect 14. A SAR of aspect 2, wherein the entire or partial extracellular antigen binding domain, the optional hinge domain, the transmembrane domain and the optional cytosolic domain are derived from two or more different naturally occurring receptor.

Aspect 15. A SAR of aspect 14, wherein a) the entire or partial extracellular antigen binding domain of a naturally occurring receptor is operationally linked to the optional hinge domain, the transmembrane domain and the optional cytosolic domain derived from one or more different naturally occurring receptors; or b) the entire or partial extracellular antigen binding domain and the optional hinge domain of a naturally occurring receptor is operationally linked to the transmembrane domain and the optional cytosolic domain derived from one or more different naturally occurring receptors; or c) the entire or partial extracellular antigen binding domain, the optional hinge and transmembrane domain of a naturally occurring receptor is operationally linked to a cytosolic domain derived from one or more different naturally occurring receptors.

Aspect 16. A SAR of aspect 2, wherein the cytosolic domain comprises an activation domain comprising ITAMs.

Aspect 17. A SAR of aspect 2, wherein the cytosolic domain lacks an activation domain comprising ITAMs.

Aspect 18. A SAR of aspect 2, wherein the cytosolic domain recruits one or more signaling adaptor selected from the group of CD3ƒ, FcRγ, DAP10 and/or DAP10.

Aspect 19. A SAR of aspect 2, wherein the cytosolic domain comprises one or more co-stimulatory domains.

Aspect 20. The SAR of aspect 2, wherein the one or more co-stimulatory domains are selected from the group consisting of CD28, 4-1BB, OX40, 2B4, CD27, CD81, CD2, CD5, BAFF-R, CD30, CD40, HVEM, ICOS, a variant thereof and a fragment thereof.

Aspect 21. A SAR of aspect 2, where the cytosolic domain lacks a co-stimulatory domain.

Aspect 22. A SAR of aspect 2, where the cytosolic domain comprises one or more co-stimulatory domains that are located between the transmembrane domain and the activation domain.

Aspect 23. A SAR of aspect 2, where the naturally occurring receptor is selected from the group consisting of CD16A, CD16B, CD64, CD32, NKp30, NKp44, NKp46, KIR2DL1, KIR2DL2, KIR2DL3, KIR2DL5A, KIR2DL5B, KIR3DL1, KIR3DL2, KIR3DL4, KIR2DL4, KIR2DS1, KIR2DS2, KIR2DS3, KIR2DS4, KIR2DS5, KIR3DS1, NKG2D, NKG2C, NKG2A, NKG2E, NKG2F, DNAM-1, 2B4, OX40, CD28, 4-1BB, CD27, CD81, CD2, CD5, TNFR-I, TNFR-II, Fas, CD30, CD40, CRTAM, TIGIT, CD96, SLAMF6, SLAMF7, CD100, CD160, CEACAM, ILT2, KLRG1, LAIR1, CD161, variants thereof and fragments thereof.

Aspect 24. A SAR of aspects 2 and 3, wherein the SAR retains partially or completely the antigen binding property of the extracellular antigen binding domain of the naturally occurring receptor and acquires the antigen binding specificities of the one or more heterologous antigen binding domains located in the first module.

Aspect 25. A SAR of aspect 1, which when expressed on the surface of a cell is able to confer MHC (or HLA)-dependent and/or MHC (or HLA)-independent antigen recognition on the cell, and wherein a) the antigen binding domain of the SAR is not comprised of a single continuous polypeptide chain; and/or b) the antigen binding domain(s) of the SAR are not derived from an antibody or an antibody fragment; and/or c) the SAR does not comprise a T cell receptor module.

Aspect 26. A SAR of aspect 25, wherein the antigen recognition domain of the SAR is derived from at least two variable domains of a TCR.

Aspect 27. A SAR of aspect 26, wherein the two variable domains comprise a heterodimer of at least two variable domains selected from Vα, Vβ, Vγ, Vδ and preTCRα.

Aspect 28. A SAR of aspect 27, wherein the two variable domains are Vα and VP or Vγ and V6.

Aspect 29. A SAR of aspect 28, wherein the two variable domains are not linked by a flexible peptide linker.

Aspect 30. A SAR of aspect 29, which is not a single chain TCR (sc-TCR).

Aspect 31. A SAR of aspect 25, which has two chain and at least one chain is membrane associated.

Aspect 32. A SAR of aspect 31, wherein both chains are membrane associated.

Aspect 33. A SAR of aspect 25, which can bind to a peptide in complex with an MHC (HLA) molecule.

Aspect 34. A SAR of aspect 25, which when expressed on the surface of a cell confers on it the ability to recruit at least one signaling adaptor when bound by a peptide/MHC complex.

Aspect 35. A SAR of aspect 25, which when expressed on the surface of a cell confers on it the ability to initiate at least one signaling pathway when bound by a peptide/MHC complex.

Aspect 36. A SAR of aspect 25, which can be functionally expressed in a non-T cell.

Aspect 37. A SAR of aspect 36, which can be functionally expressed in a cell that lacks the expression of a functional CD3 complex.

Aspect 38. A SAR of aspect 37, which can be functionally expressed in a cell that lacks the functional expression of CD3γ and CD3δ and CD3δ chains.

Aspect 39. A SAR of aspect 36, which can confer T cell like antigen recognition to a non-T cell.

Aspect 40. A SAR of aspect 36, which can confer T cell like antigen recognition to a T cell that lacks the functional expression of CD3γ and CD3δ and CD3ε chains.

Aspect 41. A SAR of aspect 36, which can confer T cell like signaling upon antigen recognition to a non-T cell.

Aspect 42. A SAR of aspect 36, which can confer T cell like signaling to a T cell that lacks the functional expression of CD3γ and CD3δ and CD3ε chains.

Aspect 43. A SAR of aspect 222, which can confer T cell like antigen recognition to any cell.

Aspect 44. A SAR of aspect 1, comprising at least two chains wherein a) a first polypeptide chain comprises a first antigen-binding domain comprising a vL, a Vα or a Vγ domain and a first Membrane associated module (MAM); and b) a second polypeptide chain comprises a second antigen-binding domain comprising a vH, a Vβ or a Vδ domain and a second Membrane associated module (MAM); wherein the vL, Vα or Vγ domain of the first antigen-binding domain and the complementary vH, Vβ or Vδ domain of the second antigen-binding domain form a Fv- or TCR-Fv like antigen-binding module that specifically binds to the target antigen; and wherein the first MAM and the second MAM form a non-T cell receptor module (NTCRM) that is capable of activating at least one signaling pathway and/or recruiting at least one signaling adaptor.

Aspect 45. The SAR of aspect 44, where the first polypeptide chain further comprises a first peptide linker between the first antigen-binding domain and the first MAM, and the second polypeptide chain further comprises a second peptide linker between the second antigen-binding domain and the second MAM.

Aspect 46. The SAR of aspect 45, wherein the first and/or second peptide linkers comprise, individually, a constant domain or fragment thereof from an immunoglobulin or T cell receptor subunit.

Aspect 47. The SAR of aspect 46, wherein the first and/or second peptide linkers comprise, individually, a CH1, CH2, CH3, CH4 or CL antibody domain, or a fragment thereof.

Aspect 48. The SAR of aspect 46, wherein the first and/or second peptide linkers comprise, individually, a Cα, Cβ, Cγ, or Cδ TCR domain, or a fragment thereof.

Aspect 49. The SAR of aspects 44 or 45, wherein the first polypeptide chain and the second polypeptide chain are linked via one or more disulfide bonds.

Aspect 50. The SAR of aspect 45, wherein first and/or second peptide linkers comprise mutations that increase the expression, affinity and/or pairing of the two polypeptide chains.

Aspect 51. The SAR of aspect 45, wherein the first and/or second peptide linkers comprise a sequence as set forth in any one of SEQ ID NO: 3536-3569 and 9627-9631, 10832-10841, and 12304-12311 or a sequence with at least 70% identity thereto.

Aspect 52. The SAR of aspect 44, wherein the first polypeptide further comprises a first hinge domain or fragment thereof N-terminal to the first MAM; and/or wherein the second polypeptide further comprises a second hinge domain or fragment thereof N-terminal to the second MAM.

Aspect 53. A SAR of aspect 44, comprising a disulfide bond between a residue in the first MAM and the second MAM and/or a residue in the first hinge domain and a residue in the second hinge domain.

Aspect 54. A SAR of aspect 44, wherein the first polypeptide further comprises a first homologous antigen binding domain or fragment thereof N-terminal to the first hinge domain and/or the second polypeptide further comprises a second homologous antigen binding domain or fragment thereof N-terminal to the second hinge domain, wherein the two homologous antigen binding domains are derived from the same naturally occurring non-T cell receptor as the corresponding hinge domains.

Aspect 55. A SAR of aspect 44, wherein the first polypeptide further comprises a first cytosolic domain containing an optional activation domain C-terminal to the first transmembrane/membrane-anchoring domain comprising the first MAM; and/or wherein the second polypeptide further comprises a second cytosolic containing an optional activation domain C-terminal to the second transmembrane/membrane anchoring domain comprising the second MAM.

Aspect 56. The SAR of aspect 44, wherein the first polypeptide chain further comprises a first accessory intracellular domain comprising a co-stimulatory domain sequence C-terminal to the first transmembrane/membrane anchoring domain of the first MAM; and/or wherein the second polypeptide chain further comprises a second accessory intracellular domain comprising a co-stimulatory domain sequence C-terminal to the second transmembrane/membrane anchoring domain comprising the second MAM.

Aspect 57. A SAR of aspect 56, wherein the co-stimulatory domain is selected from CD28, 4-1BB, OX40, 2B4, CD27, CD81, CD2, CD5, BAFF-R, CD30, CD40, HVEM or ICOS, or a variant or a fragment thereof.

Aspect 58. A SAR of aspect 44, wherein the first and/or the second MAM and the NTCRM are comprised of the transmembrane/membrane anchored domain, optional cytosolic domain, optional hinge domain and/or optional extracellular domain of a non-T cell receptor and/or a signaling adaptor.

Aspect 59. A SAR of aspect 58, wherein the first and/or the second MAM and the NTCRM are comprised of the transmembrane/membrane anchored domain, optional cytosolic domain, optional hinge domain and/or optional extracellular domain that are all derived from a single non-T cell receptor and/or a signaling adaptor or variants thereof.

Aspect 60. A SAR of aspect 58, wherein the first and/or the second MAM and the NTCRM are comprised of the transmembrane/membrane anchored domain, optional cytosolic domain, optional hinge domain and/or optional extracellular domain that are derived from different non-T cell receptor and/or a signaling adaptor or variants thereof.

Aspect 61. A SAR of aspect 58, wherein the two transmembrane/membrane anchored domains, optional cytosolic domains, optional co-stimulatory domain, optional hinge domains and/or optional extracellular domains are identical in sequence and are derived from the same protein.

Aspect 62. A SAR of aspect 58, wherein the two transmembrane/membrane anchored domains, optional cytosolic domains, optional co-stimulatory domain, optional hinge domains and/or optional extracellular domains are different in sequence and/or are derived from different proteins.

Aspect 63. A SAR of aspect 58, wherein a) the non T cell receptor is a naturally occurring receptor and is selected from the group consisting of: CD16A, CD16B, CD64, CD32, NKp30, NKp44, NKp46, KIR2DL1, KIR2DL2, KIR2DL3, KIR2DL5A, KIR2DL5B, KIR3DL1, KIR3DL2, KIR3DL4, KIR2DL4, KIR2DS1, KIR2DS2, KIR2DS3, KIR2DS4, KIR2DS5, KIR3DS1, NKG2D, NKG2C, NKG2A, NKG2E, NKG2F, DNAM-1, 2B4, OX40, CD28, 4-1BB, CD27, CD81, CD2, CD5, TNFR-I, TNFR-II, Fas, CD30, CD40, CRTAM, TIGIT, CD96, SLAMF6, SLAMF7, CD100, CD160, CEACAM, ILT2, KLRG1, LAIR1, CD161, a variant of any of the foregoing, and fragments thereof; and b) the signaling adaptor is selected from the group consisting of: CD3ƒ, FcRγ, DAP10, a variant of any of the foregoing and a fragments thereof.

Aspect 64. The SAR of aspect 44, wherein a) the first MAM and the second MAM do not comprise the transmembrane domain and optionally the cytosolic domain of a CD3 chain selected from CD3ε, CD3γ, CD3δ or CD3ƒ; and/or b)the first MAM and the second MAM do not comprise the transmembrane domain of a TCR chain and a CD3 chain; and/or c) the first MAM and the second MAM do not comprise the transmembrane domain of CD3ƒ.

Aspect 65. A SAR of aspect 44, wherein only one of the MAM is derived from a T cell receptor selected from the group consisting of TCRα, TCRβ, TCRγ, TCRδ and preTCRα.

Aspect 66. A SAR of aspect 1, comprising at least two chains wherein a) a first polypeptide chain comprises a first antigen-binding domain comprising a vL domain and a first TCR constant chain selected from TCRα, TCRβ, TCRγ or TCRδ or a variant thereof; and b) a second polypeptide chain comprises a second antigen-binding domain comprising a vH, domain and a second TCR constant chain selected from TCRα, TCRβ, TCRγ or TCRδ or a variant thereof; where the first TCR constant chain is constant chain of TCRα and the second TCR constant chain is constant chain of TCRβ, or where the first TCR constant chain is constant chain of TCRβ and the second TCR constant chain is constant chain of TCRα, or where the first TCR constant chain is constant chain of TCRγ and the second TCR constant chain is constant chain of TCRδ, or where the first TCR constant chain is constant chain of TCRδ and the second TCR constant chain is constant chain of TCRγ, or wherein the first TCR constant chain and/or the second TCR constant chain lacks amino acid residues at its N-terminal region wherein the vL and the vH domain form a Fv like antigen-binding module that specifically binds to a target antigen; and wherein the first TCR constant chain and the second TCR constant chain form a T cell receptor module (TCRM) that is capable of activating at least one signaling pathway and/or recruiting at least one signaling adaptor.

Aspect 67. A SAR of aspect 66, wherein a) the TCRα constant chain is represented by an amino acid sequence with SED ID NO (PRT): 7863-7963 or sequences with 80-99% homology thereto; and b) the TCRβ constant chain is represented by an amino acid sequence with SED ID NO (PRT): 7964-8089 or sequences with 80-99% homology thereto; and c) the TCRγ constant chain is represented by an amino acid sequence with SED ID NO (PRT): 8091-8191 or sequences with 80-99% homology thereto; and d) the TCRδ constant chain is represented by an amino acid sequence with SED ID NO (PRT): 8192-8292 or sequences with 80-99% homology thereto.

Aspect 68. A SAR of aspect 44 or 66, wherein the first and/or the second polypeptide chains further comprise one or more autonomous antigen binding domains (AABD) that are attached to the N-terminus or near the N-terminus of the first and/or the second antigen binding domains.

Aspect 69. The SAR of aspect 68, wherein the AABD is selected from one or more of a single vH domain (SVH), a single vL domain (SVL), a vHH domain, a single domain antibody, a single variable domain of a TCR (svd-TCR), a non-immunoglobulin antigen binding scaffold, a ligand-binding domain of a receptor, a receptor-binding domain of a ligand, an autoantigen, an adaptor binding domain, an Fc binding domain, a fragment thereof and/or a variant thereof.

Aspect 70. The SAR of aspect 1, wherein the module comprising one or more heterologous antigen-binding domains binds specifically to one or more target antigens selected from the group consisting of a) cell surface protein antigen, b) peptide/MHC complex, and c) lipid antigen.

Aspect 71. The SAR of aspect 1, wherein the target antigen is selected from the group consisting of: CD19; CD123; CD22; CD30; CD171; CS-1 (also referred to as CD2 subset 1, CRACC, SLAMF7, CD319, and 19A24); C-type lectin-like molecule-1 (CLL-1 or CLECL1); CD33; epidermal growth factor receptor variant III (EGFRviii); ganglioside G2 (GD2); ganglioside GD3; TNF receptor family member B cell maturation (BCMA); Tn antigen ((Tn Ag); prostate-specific membrane antigen (PSMA); Receptor tyrosine kinase-like orphan receptor 1 (ROR1); FmsLike Tyrosine Kinase 3 (FLT3); Tumor-associated glycoprotein 72 (TAG72); CD38; CD44v6; a glycosylated CD43 epitope expressed on acute leukemia or lymphoma but not on hematopoietic progenitors, a glycosylated CD43 epitope expressed on non-hematopoietic cancers, Carcinoembryonic antigen (CEA); Epithelial cell adhesion molecule (EPCAM); B7H3 (CD276); KIT (CD 117); Interleukin-13 receptor subunit alpha-2 (IL-13Ra2 or CD213A2); Mesothelin; Interleukin 11 receptor alpha (IL-llRa); prostate stem cell antigen (PSCA); Protease Serine 21 (Testisin or PRSS21); vascular endothelial growth factor receptor 2 (VEGFR2); Lewis(Y) antigen; CD24; Platelet-derived growth factor receptor beta (PDGFR-beta); Stage-specific embryonic antigen-4 (SSEA-4); CD20; Folate receptor alpha; Receptor tyrosine-protein kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CAlX); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); Fucosyl GM1; sialyl Lewis adhesion molecule (sLe); ganglioside GM3; transglutaminase 5 (TGS5); high molecular weight-melanoma associated antigen (HMWMAA); o-acetyl-GD2 ganglioside (OAcGD2); Folate receptor beta; tumor endothelial marker 1 (TEM1/CD248); tumor endothelial marker 7-related (TEM7R); claudin 6 (CLDN6); thyroid stimulating hormone receptor (TSHR); G protein coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); Cancer/testis antigen 1 (NY-ESO-1); Cancer/testis antigen 2 (LAGE-la); Melanoma-associated antigen 1 (MAGE-A1); MAGE-A2, MAGE-A3, MAGE-A4, PRAME, PSA, ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p5³); p53 mutant; prostein; surviving; telomerase; prostate carcinoma tumor antigen-1 (PCT A-1 or Galectin 8), melanoma antigen recognized by T cells 1 (MelanA or MARTI); Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin Bl; v-myc avian myelocytomatosis viral oncogene neuroblastoma derived homolog (MYCN); Ras Homolog Family Member C (RhoC); Tyrosinase-related protein 2 (TRP-2); Cytochrome P450 lB 1 (CYPlB 1); CCCTC-Binding Factor (Zinc Finger Protein)-Like (BORIS or Brother of the Regulator of Imprinted Sites), Squamous Cell Carcinoma Antigen Recognized By T Cells 3 (SART3); Paired box protein Pax-5 (PAX5); proacrosin binding protein sp32 (OY-TESl); lymphocyte-specific protein tyrosine kinase (LCK); A kinase anchor protein 4 (AKAP-4); synovial sarcoma, X breakpoint 2 (SSX2); Receptor for Advanced Glycation End products (RAGE-1); renal ubiquitous 1 (RUl); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIRl); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); and immunoglobulin lambda-like polypeptide 1 (IGLLl), MPL, Biotin, c-MYC epitope Tag, CD34, LAMP1 TROP2, GFRalpha4, CDH17, CDH6, NYBR1, CDH19, CD200R, Slea (CA19.9; Sialyl Lewis Antigen) Fucosyl-GM1, PTK7, gpNMB, CDH1-CD324, DLL3, CD276/B7H3, IL1lRa, IL13Ra2, CD179b-IGLll, ALK TCRgamma-delta, NKG2D, CD32 (FCGR2A), Tn ag, CSPG4-HMW-MAA, Tim1-/HVCR1, CSF2RA (GM-CSFR-alpha), TGFbetaR2, VEGFR2/KDR, Lews Ag, TCR-beta1 chain, TCR-beta2 chain, TCR-gamma chain, TCR-delta chain, FITC, Leutenizing hormone receptor (LHR), Follicle stimulating hormone receptor (FSHR), Chorionic Gonadotropin Hormone receptor (CGHR), CCR4, GD3, SLAMF6, SLAMF4, HIV1 envelope glycoprotein, HTLV1-Tax, CMV pp65, EBV-EBNA3c, influenza A hemagglutinin (HA), GAD, PDL1, Guanylyl cyclase C (GCC), auto antibody to desmoglein 3 (Dsg3), autoantibody to desmoglein 1 (Dsgl), HLA, HLA-A, HLA-A2, HLA-B, HLA-C, HLA-DP, HLA-DM, HLA-DOA, HLA-DOB, HLA-DQ, HLA-DR, HLA-G, IGE, CD99, RAS G12V, Tissue Factor 1 (TF1), AFP, GPRC5D, claudin18.2 (CLD18A2 OR CLDN18A.2), P-glycoprotein, STEAPI, LIV1, NECTIN-4, CRIPTO, GPA33, BST1/CD157, low conductance chloride channel, and SARS-CoV2 Spike protein.

Aspect 72. The SAR of aspect 1, wherein the encoded SAR polypeptide comprises one or more heterologous antigen binding domains selected from the group consisting of: (i) a heavy chain variable region (vH) comprising a sequence as set forth in any of SEQ ID Nos: 2682-2918 or sequences with at least 80% identity thereto or a sequence with at least 80% identity in the three complementarity determining regions (CDRs) to the sequences set forth in any one or more of SEQ ID NOS: 2682-2918 or a sequence with less than 3 substitutions in the three CDRs of the sequences set forth in any one or more of SEQ ID NOS: 2682-2918 or a sequence with less than three substitutions in the CDR1, CDR2 and CDR3 that belong to a vH and are presented in SEQ ID NO: 11593-11829, 11830-12066, 12067-12303, respectively, or a sequence that binds to the same target antigens or the same epitopes on the target antigens as a sequence set forth in any one or more of SEQ ID NOS: 2682-2918 and which encodes a polypeptide that binds to its antigen; (ii) a light chain variable region (vL) comprising a sequence as set forth in any one of SEQ ID NO: 2440-2676 or sequences with at least 80% identity to sequences set forth in any one or more of SEQ ID NOS: 2440-2676 or a sequence with at least 80% identity in the three complementarity determining regions (CDRs) to the sequences set forth in any one or more of SEQ ID NOS: 2440-2676 or a sequence with less than 3 substitutions in the three CDRs of the sequences set forth in any one or more of SEQ ID NOS: 12440-2676 or a sequence with less than three substitutions in the CDR1, CDR2 and CDR3 that belong to a vL and are presented in SEQ ID NO: 10882-11118, 11119-11355 and 11356-11592, respectively, or a sequence that binds to the same target antigens or the same epitopes on the target antigens as a sequence set forth in any one or more of SEQ ID NOS: 2440-2676 and which encodes a polypeptide that binds to its antigen; (iii) a single chain variable fragment (scFv) comprising a sequence as set forth in any one SEQ ID NO: 2924-3160 or a sequence with at least 80% identity thereto or a sequence with at least 70% identity in the six complementarity determining regions (CDRs) to the sequences set forth in any one or more of SEQ ID NOS: 2924-3160 or a sequence with less than 6 substitutions in the six CDRs of the sequences set forth in any one or more of SEQ ID NOS: 2924-3160 or a sequence with less than three substitutions in the CDR1, CDR2 and CDR3 that belong to a vH comprising a scFV and are presented in SEQ ID NO: 11593-11829, 11830-12066, 12067-12303, respectively and less than thre substitutions in the light chain CDR1, CDR2 and CDR3 that belong to a vL comprising a scFv and are presented in SEQ ID NO: 10882-11118, 11119-11355 and 11356-1159 respectively, or a sequence that binds to the same target antigens or the same epitopes on the target antigens as a sequence set forth in any one or more of SEQ ID NOS: 2924-3160 and which encodes a polypeptide that binds to its antigen; (iv) a single domain antibody, a vHH domain, a SVH, and/or FHVH domain comprising a sequence as set forth in any one of SEQ ID NO: 3210-3353, 10695-10713 or a sequence with at least 70% identity to a sequence set forth in any one or more of SEQ ID NOS: 3210-3353, 10695-10713 and or a sequence with at least 70% identity in the three complementarity determining regions (CDRs) to the sequences set forth in any one or more of SEQ ID NOS: 3210-3353, 10695-10713 or a sequence with less than 3 substitutions in the three CDRs of the sequences set forth in any one or more of SEQ ID NOS: 3210-3353, 10695-10713 or a sequence that bind to the same target antigens or the same epitopes on the target antigens as a sequence set forth in any one or more of SEQ ID NOS: 3210-3353, 10695-10713 and which encodes a polypeptide that binds to its antigen; (v) a non-immunoglobulin scaffold encoded by a polynucleotide of any one of SEQ ID NOS: 3366-3377 or sequences with at least 70% identity to sequences set forth in any one or more of SEQ ID NOS: 3366-3377 or sequences that bind to the same target antigens or the same epitopes on the target antigens as the sequences set forth in any one or more of SEQ ID NOS: 3366-3377; (vi) the ligand binding domain of a receptor comprising a sequence as set forth in any one of SEQ ID NO: 3378-3395, 3880, 3882, 3886, 3893, 3896, 3897 or sequences with at least 70% identity thereto and which encodes a polypeptide that binds to its cognate; (vii) the receptor binding domain of a ligand comprising a sequence as set forth in any one of SEQ ID NO: 3396-3406, 10786-10787 or sequences with at least 70% identity thereto and which encodes a polypeptide that binds to its cognate; (viii) an adaptor binding domain comprising a sequence as set forth in any one of SEQ ID NO: 3407-3435, 10771-10780 or sequences with at least 70% identity thereto and which encodes a polypeptide that binds to its adaptor; (ix) an autoantigen comprising a sequence as set forth in any one of SEQ ID NO 10788-10791 or sequences with at least 70% identity thereto and which encodes a polypeptide that binds to its autoantibody or autoantibody producing cells; (x) a TCR variable region (Va, Vb, Vg or Vd) comprising a sequence as set forth in any of SEQ ID NOs: 3357-3364, 9606-9614, 10781-10782 or sequences with at least 70% identity thereto or sequences with at least 70% identity in the three complementarity determining regions (CDRs) to the sequences set forth in any one or more of SEQ ID NOS: 3357-3364, 9606-9614, 10781-10782 or sequences with less than 3 substitutions in the three CDRs of the sequences set forth in any one or more of SEQ ID NOS: 3357-3364, 9606-9614, 10781-10782 or sequences that bind to the same target antigens or the same epitopes on the target antigens as the sequences set forth in any one or more of SEQ ID NOS: 3357-3364, 9606-9614, 10781-10782 and which encodes a polypeptide that binds to its antigen; and (xi) a single variable TCR domain (svd-TCR) comprising a sequence as set forth in any of SEQ ID NOs: 9613-9614 or sequences with at least 70% identity thereto or sequences with 70-99% identity in the three complementarity determining regions (CDRs) to the sequences set forth in any one or more of SEQ ID NOS: 9613-9614 or sequences with less than 3 substitutions in the three CDRs of the sequences set forth in any one or more of SEQ ID NOS: 9613-9614 or sequences that bind to the same target antigens or the same epitopes on the target antigens as the sequences set forth in any one or more of SEQ ID NOS: 9613-9614 and which encodes a polypeptide that binds to its antigen.

Aspect 73. A SAR of aspect 2 or 58, wherein a naturally occurring receptor and/or the signaling adaptor or a fragment thereof comprises a sequence selected from SEQ ID NO: 3743-3966, 3385, 3394, 7818-7822, 9633-9859 or a sequence with 70% homology to a sequence thereto.

Aspect 74. A SAR of aspect 2 or 58, wherein a polypeptide comprising the hinge domain, transmembrane domain and cytosolic domains of naturally occurring receptor and/or the signaling adaptor comprises a sequence selected from SEQ ID NO: 9669-9704, 3813, 8721, 8733 and 8746 or a sequence with 70% homology to a sequence thereto.

Aspect 75. A SAR of aspect 1, 2, or 58, wherein a membrane associated domain of a naturally occurring receptor and/or a signaling adaptor comprises a sequence selected from SEQ ID NO: 3914-3928, 9741-9776, 9852-9855 or a sequence with 70% homology to a sequence thereto.

Aspect 76. A SAR of aspects 1, 2 or 58, wherein a cytosolic domain of a naturally occurring receptor and/or a signaling adaptor comprises a sequence selected from SEQ ID NO: 3944-3958, 9777-9812, 9856-9859 or a sequence with 70% homology to a sequence thereto.

Aspect 77. A SAR of aspects 16 or 55, wherein the activation domain of a naturally occurring receptor and/or a signaling adaptor comprises a sequence selected from SEQ ID NO: 9856-9859 and 9777 or a sequence with 70% homology to a sequence thereto.

Aspect 78. A SAR of aspect 9, 48 and 56, wherein the co-stimulatory domain comprises a sequence selected from SEQ ID NO: 9807-9810 or a sequence with 70% homology to a sequence thereto.

Aspect 79. The SAR of aspect 1, further comprising a leader sequence or signal peptide that is present at the N-terminal of each chain and optionally comprising a sequence selected from the group consisting of SEQ ID NO:2425-2430.

Aspect 80. The isolated SAR polypeptide aspect 1, wherein the SAR comprises a SAR heterodimer.

Aspect 81. The isolated synthetic antigen receptor (SAR) polypeptide or polypeptide heterodimer of aspects 1 or 80, where the polypeptide comprises two SAR chains that are linked by a cleavable linker.

Aspect 82. The isolated synthetic antigen receptor (SAR) polypeptide or polypeptide heterodimer of aspects 81, wherein the cleavable linker is a self-cleaving cleavable linker.

Aspect 83. The isolated synthetic antigen receptor (SAR) polypeptide or polypeptide heterodimer of aspect 82, wherein the cleavable linker is any one or more of a 2A linker, a 2A-like linker or functional equivalent thereof.

Aspect 84. The isolated synthetic antigen receptor (SAR) polypeptide or polypeptide heterodimer of aspect 83, wherein the cleavable linker is any one or more of T2A linker, P2A, F2A, E2A linker or functional equivalent thereof.

Aspect 85. The isolated synthetic antigen receptor (SAR) polypeptide or polypeptide heterodimer of aspect 84, wherein the cleavable linker comprises a sequence of any one or more of SEQ ID Nos: 3627-3632.

Aspect 86. The isolated synthetic antigen receptor (SAR) polypeptide or polypeptide heterodimer of aspect 84, wherein the cleavable linker is optionally preceded by a furine cleavage site or furine like cleavage site or functional equivalent thereof.

Aspect 87. The isolated synthetic antigen receptor (SAR) polypeptide or polypeptide heterodimer of aspect 86, wherein the furine cleavage site preceding the cleavable linker comprises a sequence of any one or more of SEQ ID Nos:3635-3636.

Aspect 88. The isolated synthetic antigen receptor (SAR) polypeptide or polypeptide heterodimer of any one of aspects 86-87, wherein the cleavable linker is preceded by a flexible linker.

Aspect 89. The isolated synthetic antigen receptor (SAR) polypeptide or polypeptide heterodimer of aspect 88, wherein the flexible linker preceding the cleavable linker encodes for one or more of Ser-Gly linker or functional equivalent thereof.

Aspect 90. The isolated synthetic antigen receptor (SAR) polypeptide or polypeptide heterodimer of aspect 89, wherein the flexible linker preceding the cleavable linker comprises a sequence of SEQ ID Nos: 3633-3634.

Aspect 91. The isolated synthetic antigen receptor (SAR) polypeptide or polypeptide heterodimer of any one of aspects 88-90, wherein the furine cleavage site is followed by the flexible linker which is followed by the cleavable linker so that the order is Furine cleavage site-Flexible linker-cleavable linker.

Aspect 92. The isolated synthetic antigen receptor (SAR) polypeptide or polypeptide heterodimer of aspect 1 or 80, wherein the SARs is designed to have a desired binding affinity for a selected antigen.

Aspect 93. A SAR of aspect 1, further expressing an accessory module comprising a polypeptide that is selected from the group consisting of: a) a cytokine or a variant thereof; b) membrane-anchored cytokine; c) a membrane anchored cytokine with epitope tags; d) a multi-purpose switch that serves as a suicide, survival and marker function; e) a signaling adaptor molecule; and f) a kill-switch.

Aspect 94. An accessory module of aspect 93, wherein a) the cytokine comprises a sequence with SEQ ID NO:7833-7842 or a variant with up to 70% sequence homology thereto, and b) the membrane-anchored cytokine comprises a sequence with SEQ ID NO: 7825-7832 or a variant with up to 70% sequence homology thereto, c) the multipurpose switch comprises a sequence with SEQ ID NO: 7843-7850 or a variant with 70% sequence homology thereto, and d) the adaptor is selected from the group of CD3ƒ, FcRγ, DAP10 and DAP12.

Aspect 95. A polypeptide comprising a multi-purpose switch of aspect 94 having the formula: SP-D1-L1-D2-L2-D3-L3-D4; wherein SP is an optional signal peptide that allows cell surface transport of the multipurpose switch and is cleaved to yield the mature peptide, D1 is receptor binding domain which binds to a receptor that promotes cell survival, D2 is a marker/suicide domain, D3 is a hinge domain/stalk domain that allows the D1 and D2 domains to be projected away from the surface of the target cell, D4 is a membrane associating domain that anchors the multi-purpose switch to the cell membrane, and L1, L2 and L3 are optional linker.

Aspect 96. A polypeptide of aspect 95, wherein the multipurpose switch polypeptide comprises an in-frame fusion of a first module (D1), (D2), (D3) and (D4).

Aspect 97. A polypeptide of aspect 96, wherein a) D3 and D4 modules are derived from the same endogenous protein; or b) D2, D3 and D4 module are derived from different endogenous proteins; or c) D3 and D4 are derived from the same endogenous protein; or d) D3 and D4 are derived from different endogenous proteins.

Aspect 98. A polypeptide of aspect 95, wherein the first module (D1) binds to a receptor that is expressed on the cell surface.

Aspect 99. A polypeptide of aspect 98, wherein the receptor when bound transmits a pro-survival and/or proliferative signal to the cell.

Aspect 100. A polypeptide of aspect 98, wherein the first module binds to the receptor in cis and/or the first module binds to the receptor in trans.

Aspect 101. A polypeptide of aspect 95, wherein the first module (D1) comprises the receptor binding domain of a cytokine, a chemokine, a ligand, or a variant or a fragment thereof.

Aspect 102. A multipurpose switch of aspect 101, wherein the D1 comprises the receptor binding domain of a cytokine, a chemokine or a ligand selected from the group consisting of IL2, IL4, IL6, IL7, IL9, IL10, IL11, IL12, IL15, IL18, IL21, CD40L, 4-1BBL, CD30L, OX40L, FLT3-L, APRIL, BAFF, Rantes, MIP, Erythropoietin, Thrombopoietin, SCF (stem cell factor), G-CSF, GM-CSF, M-CSF, a variant of any of the foregoing and a fragment of any of the foregoing.

Aspect 103. A polypeptide of aspect 101, wherein the D1 comprises a polypeptide with sequence represented by SEQ ID NO: 7833 to 7842 or a variant with at least 70% identity thereto.

Aspect 104. A polypeptide of aspect 95, wherein the D1 comprises an antibody, an antibody fragment, a single domain antibody, a single chain antibody, an scFv, or a non-immunoglobulin antigen binding module that can bind to a receptor.

Aspect 105. A polypeptide of aspect 95 and 104, wherein the D1 binds to a receptor selected from the group consisting of: IL2R, IL6R, IL7R, IL9R, IL10R, IL11R, IL12R, IL15R, IL18, IL21 CCR1, CCR3, CCR5, MIP-1R, PF4 receptor, Erythropoeitin-Receptor (Epo-R), TPO-R/MPL, GSF-R, c-Kit, and M-CSF receptor.

Aspect 106. A polypeptide of aspect 95, wherein the D2 comprises of a non-endogenous polypeptide.

Aspect 107. A polypeptide of aspect 95, wherein the D2 comprises of the extracellular domain of an endogenous protein or a variant or a fragment thereof.

Aspect 108. A polypeptide of aspect 95, wherein the D2 comprises extracellular domain of one or more of the following endogenous proteins or a variant or a fragment thereof: CD5; CD19; CD123; CD22; CD30; CD3ε, CD52, CD171; CS1 (SLAMF7, CD319); C-type lectin-like molecule-1 (CLL-1 or CLECL1); CD33; epidermal growth factor receptor variant III (EGFRviii); ganglioside G2 (GD2); ganglioside GD3; BCMA; Tn antigen (Tn Ag); prostate-specific membrane antigen (PSMA); Receptor tyrosine kinase-like orphan receptor 1 (ROR1); Fins Like Tyrosine Kinase 3 (FLT3); Tumor-associated glycoprotein 72 (TAG72); CD38; CD44v6; Carcinoembryonic antigen (CEA); Epithelial cell adhesion molecule (EPCAM); B7H3 (CD276); KIT (CD 117); Interleukin-13 receptor subunit alpha-2 (IL-13Ra2 or CD213A2); Mesothelin; Interleukin 11 receptor alpha (IL-llRa); prostate stem cell antigen (PSCA); Protease Serine 21 (Testisin or PRSS21); vascular endothelial growth factor receptor 2 (VEGFR2); Lewis(Y) antigen; CD24; Platelet-derived growth factor receptor beta (PDGFR-beta); Stage-specific embryonic antigen-4 (SSEA-4); CD20; Folate receptor alpha (FRa or FR1); Folate receptor beta (FRb); Receptor tyrosine-protein kinase ERBB2 (Her2/neu); Mucin 1, cell surface associated (MUC1); epidermal growth factor receptor (EGFR); neural cell adhesion molecule (NCAM); Ephrin B2; fibroblast activation protein alpha (FAP); insulin-like growth factor 1 receptor (IGF-I receptor), carbonic anhydrase IX (CAlX); ephrin type-A receptor 2 (EphA2); sialyl Lewis adhesion molecule (sLe); ganglioside GM3; high molecular weight-melanoma associated antigen (HMWMAA); o-acetyl-GD2 ganglioside (OAcGD2); tumor endothelial marker 1 (TEM1/CD248); tumor endothelial marker 7-related (TEM7R); claudin 6 (CLDN6); thyroid stimulating hormone receptor (TSHR); G protein coupled receptor class C group 5, member D (GPRC5D); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Androgen receptor; Squamous Cell Carcinoma Antigen Recognized By T Cells 3 (SART3); CD79a; CD79b; CD72; Leukocyte-associated immunoglobulin-like receptor 1 (LAIRl); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); and immunoglobulin lambda-like polypeptide 1 (IGLLl), MPL, CD34, LAMP1 TROP2, GFRalpha4, CDH17, CDH6, NYBRI, CDH19, CD200R, Slea (CA19.9; Sialyl Lewis Antigen); Fucosyl-GM1, PTK7, gpNMB, CDH1/CD324, DLL3, CD276/B7H3, IL-2R, IL-4R, IL-6R, IL1lRa, IL13Ra2, IL-17R, CD179b-IGLll, TCRgamma-delta, NKG2D, CD32 (FCGR2A), Timl-/HVCR1, CSF2RA (GM-CSFR-alpha), TGFbetaR2, Lews Ag, TCR-beta1 chain, TCR-beta2 chain, TCR-gamma chain, TCR-delta chain, FITC, Leutenizing hormone receptor (LHR), Follicle stimulating hormone receptor (FSHR), Gonadotropin Hormone receptor (CGHR or GR), CCR4, SLAMF6, SLAMF4, CD99, Ras G12V, Tissue Factor 1 (TF1), GPRC5D, Claudin18.2 (CLD18A2 or CLDN18A.2), P-glycoprotein, STEAPI, Livl, Nectin-4, Cripto, gpA33, BST1/CD157, low conductance chloride channel (LCCC), TAJ/TROY, MPL (TPO-R), KIR3DL2, CD32b, CD229, Toso, PD-1, PD-L1, PD-L2, TNFRI, TRAIL-Ri (DR4), TRAIL-R2 (DR5), CTLA4, IL-36R, CD25, LAG3, VEGF-A, MASP-2, Thymic stromal lymphopoietin, Tissue Factor, IFNARI, IL5, IL-6, IL-12, IL-23, IL-17A, IL-13, Angiopoietin-like 3, CGRP, IL-23pi9, vWF, C5, IFNy, CD4, CD8, CD7, NKp30, NKp44, NKp46, NKG2D, PDGRFα, α4β7 integrin, α4 integrin, VEGF, GPIIb/IIIa PCSK9, Blys, and BAFF-R.

Aspect 109. A polypeptide of aspect 95, wherein the D2 can be bound by agent that can be used to detect, enrich and/or kill the cells expressing the multi-purpose switch.

Aspect 110. A polypeptide of aspect 109, wherein the agent is selected from one or more of: an antibody, an antibody fragment, an scFv, a single domain antibody, a non-immunoglobulin antigen binding domain, an antibody drug conjugate, a bispecific antibody or a fragment thereof or a cell.

Aspect 111. A polypeptide of aspect 110, wherein the agent that binds D2 is approved for in vivo or ex vivo human clinical use.

Aspect 112. The polypeptide of aspect 111, wherein the agent is selected from the group consisting of Rituximab, Herceptin, Enhertu, Erbitrux, Adcetris, Enbrel, Tremelimumab, Mosunetuzumab, Teclistamab, Donanemab, Spesolimab, Faricimab, belantamab mafodotin, Tislelizumab, loncastuximab tesirine, Tafasitamab, Pembrolizumab, nivolumab and Qbend10.

Aspect 113. A polypeptide of aspect 95, wherein the D3 comprises a stalk (hinge domain) sequence that is between 5-100 amino acids in length.

Aspect 114. A polypeptide of aspect 95, comprising an amino acid sequence represented by SEQ ID NO (PRT): 7843-7850, SEQ ID NO (PRT): 9625 and SEQ ID NO: 9620-9624 or a variant with at least 80% homology thereto.

Aspect 115. A polypeptide according to aspect 95, which comprises a sequence shown as SEQ ID No. 7843-7849, or a variant thereof which has at least 80% identity with the sequence shown as SEQ ID No. 7843-7849 and which (i) binds J6M0; (ii) binds belantamab mafodotin and (iii) when expressed on the surface of a T cell or an NK cell, promotes survival; (v) when expressed on the surface of a T cell or an NK cell, induces killing of the cell in the presence of belantamab mafodotin.

Aspect 116. A polypeptide according to aspect 95, which comprises a sequence shown as SEQ ID No. 9620-9624, or a variant thereof which has at least 80% identity with the sequence shown as SEQ ID No. 9620-9624 and which (i) binds QBEND10; (ii) binds Rituximab and (iii) when expressed on the surface of a T cell or an NK cell, promotes survival; (v) when expressed on the surface of a T cell or an NK cell, induces complement-mediated killing of the cell in the presence of Rituximab.

Aspect 117. A polypeptide according to aspect 95, which comprises a sequence shown as SEQ ID No. 9625, or a variant thereof which has at least 80% identity with the sequence shown as SEQ ID No. 9625 and which (i) binds Herceptin; (ii) binds and Enhertu (iii) when expressed on the surface of a T cell or an NK cell, promotes survival; (v) when expressed on the surface of a T cell or an NK cell, induces killing of the cell in the presence of Herceptin or Enehertu.

Aspect 118. A recombinant nucleic acid(s) encoding the first and/or second polypeptide chains of the SAR of aspect 1 and/or one or more accessory modules of aspect 93.

Aspect 119. A recombinant expression system comprising the recombinant polynucleotide of aspect 118, which is co-expressed with an accessory, wherein the accessory module is selected from the group consisting of a truncated epidermal growth factor receptor (tEGFR), truncated epidermal growth factor receptor viii (tEGFRviii), truncated CD30 (tCD30), truncated BCMA (tBCMA), truncated CD19 (tCD19), CD34, thymidine kinase, cytosine deaminase, nitroreductase, xanthine-guanine phosphoribosyl transferase, human caspase 8, human caspase 9, inducible caspase 9 (icaspase9), purine nucleoside phosphorylase, linamarase/linamarin/glucose oxidase, deoxyribonucleoside kinase, horseradish peroxidase (HRP)/indole-3-acetic (IAA), Gamma-glutamylcysteine synthetase, CD20/alphaCD20, CD34/thymidine kinase chimera, dox-dependent caspase-2, mutant thymidine kinase (HSV-TKSR39), AP1903/Fas system, a chimeric cytokine receptor (CCR), a selection marker, a multi-purpose switch, vFLIP-K13, vFLIP-MC159, 4-1BBL-CD40L, DAP10, DAP12, NKG2C, CD94, CD3ε, CD3γ, CD36, CD3ƒ, FcRγ, dihydroxyfolate receptor (DHFR), mutant DHFR, methylated-DNA-protein-cysteine methyltransferase, inosine monophosphate dehydrogenase II (IMDHP2), puromycin acetyl transferase (PAC), blasticidin-resistance gene, mutant calcinueurin a/b (Can/b), CNa12, CNb30 and combinations thereof.

Aspect 120. The recombinant expression system of aspect 119, wherein the recombinant polynucleotide encoding the one or two chains of the SAR and one or more accessory modules are linked by nucleotide sequences encoding an optional flexible linker, an optional furine cleavage site or furine like cleavage site and a cleavable linker.

Aspect 121. The recombinant expression system of aspect 120, wherein the recombinant polynucleotide encoding the one or two chains of the SAR and one or more accessory modules are expressed using i) one or more promoters; ii) one or more Internal ribosomal entry sites (IRES); iii) one or more cleavable linkers; iv) any combination of i, ii and iii.

Aspect 122. A recombinant expression system of aspect 121, wherein a) the promoter is an MNDU3 promoter, EFlu promoter, EFS promoter (SEQ ID NO: 8505), EFS2 promoter (SEQ ID NO: 8506), RSV promoter (SEQ ID NO:8507), or mutRSV promoter (SEQ ID NO: 8508) or sequences with 70% identity thereto; and b) the IRES is K-IRES (SEQ ID NO: 8504) or a sequence with 70% identity thereto.

Aspect 123. At least one vector comprising the recombinant polynucleotide of aspect 118 and recombinant expression system of aspect 119, wherein the vector is selected from the group consisting of a DNA vector, an RNA vector, a plasmid, a lentivirus vector, adenoviral vector, a retrovirus vector, a baculovirus vector, a sleeping beauty transposon vector, and a piggybac transposon vector.

Aspect 124. The vector of aspect 123, comprising one or more constitutive promoters or regulatable promoters.

Aspect 125. The vector of aspect 104, where the promoter is chosen from an MNDU3 promoter, EFlu promoter, EFS promoter (SEQ ID NO: 8505), EFS2 promoter (SEQ ID NO: 8506), RSV promoter (SEQ ID NO:8507), or mutRSV promoter (SEQ ID NO: 8508), a CMV IE gene promoter, an EF-la promoter, a ubiquitin C promoter, a MSCV LTR promoter, a phosphoglycerate kinase (PGK) promoter or a synthetic Notch (SynNotch) promoter.

Aspect 126. The vector of aspect 123, wherein the vector is an in vitro transcribed vector, or the vector further comprises a poly(A) tail or a 3′UTR.

Aspect 127. An effector cell or stem cell comprising at least one SAR polypeptide or heterodimer of aspect 1, a nucleic acid of aspect 118, an optional accessory module, a recombinant expression system of aspect 119, and a vector of aspect 123.

Aspect 128. The effector cell or stem cell of aspect 127, wherein the cell comprises a plurality of single or double chain SAR polypeptides.

Aspect 129. The effector cell or stem cell of aspect 128, wherein at least one single or double chain SAR polypeptide of the plurality of SAR polypeptides targets a different antigen than at least one other SAR polypeptide.

Aspect 130. The effector cell or stem cell of aspect 127, wherein at least one SAR polypeptide of the plurality of SAR polypeptides target the same antigen.

Aspect 131. The effector cell or stem cell of aspect 127, wherein at least one SAR polypeptide of the plurality of SAR polypeptides comprises a different binding affinity for the antigen than at least one other SAR polypeptide.

Aspect 132. The effector cell or stem cell of aspect 127, wherein at least one SAR polypeptide of the plurality of SAR polypeptides comprises a different naturally occurring receptor or a signaling adaptor than at least one other SAR polypeptide.

Aspect 133. The effector cell or stem cell of aspect 127, wherein the least one SAR polypeptide of the plurality of SAR polypeptides has a different extracellular domain, transmembrane domain, cytosolic domain than at least one other SAR polypeptide.

Aspect 134. The effector cell or stem cell of aspect 127, wherein the least one SAR polypeptide of the plurality of SAR polypeptides is an activating receptor and at least one other SAR polypeptide is an inhibitory receptor.

Aspect 135. The effector cell or stem cell of aspect 127, wherein the two or more SAR polypeptide of the plurality of SAR polypeptides are activating receptors or two or more SAR polypeptide of the plurality of SAR polypeptides are inhibitory receptors.

Aspect 136. The effector cell or stem cell of aspect 127, wherein the two or more SAR polypeptide of the plurality of SAR polypeptides recruit different signaling adaptors and/or activate different signal transduction pathways.

Aspect 137. The effector cell or stem cell of any one of aspects 127-136, wherein the effector cell is a a/P T cell, y/⁶ T cell, CD8⁺ T cell, a CD4⁺ T cell, a memory T cell, naïve T cell, T stem cell, a Treg cell, natural killer T (NKT) cell, iNKT (innate natural killer cell), NK cell, g-NK cell, memory like NK cells, cytokine induced killer cell (CIK), iPSC, a modified HLA deficient iPSC, iPSC-derived NK cell, iPSC-derived T cell, B cell, a macrophage/monocyte, granulocyte, a dendritic cell, an immortalized cell line, an immortalized NK cell line, NK92 cell line, NK92MI cell line, YTS cell or derivative thereof.

Aspect 138. A population of effector cells of any one of aspects 127-137, wherein the population of cells comprises a plurality of diverse SAR polypeptides.

Aspect 139. The population of immune or effector cells of aspect 138, wherein the plurality of diverse SAR polypeptides comprise different sequences but bind to the same target antigen or different antigens.

Aspect 140. A method of making a SAR-expressing effector cell of aspect 127, comprising introducing at least one vector of aspect 123 or at least one recombinant polynucleotide of aspect 118 into an effector cell, a cell line, a hematopoietic stem cell, a progenitor cell or an IPSC that can give rise to an effector cell, under conditions such that the SAR polypeptide and the optional accessory module are expressed.

Aspect 141. The effector cell of aspect 127, wherein the effector cell lacks expression or has low expression of a functional TCR, a functional HLA, 32 macroglobulin, TAP1, TAP2, tapasin, NLRC5, CIITA, RFXANK, CIITA, RFX5, RFXAP, TCRα or R constant region, NKG2A, NKG2D, CD3ε, CD5, CD52, CD33, CD123, CLL-1, CIS, CBL-B, SOCS2, PD1, CTLA4, LAG3, TIM3, TIGIT, or any gene in the chromosome 6p21 region; and/or introduced or increased expression in at least one of HLA-E, 41BBL, CD3ε, CD3γ, CD36, CD3ƒ, FcRγ, DAP10, DAP12, CD4, CD8, CD16, CD47, CD94, CD113, CD131, CD137, CD80, PDL1, A2AR, Fc receptor, an engager, or surface triggering receptor for coupling with bi- or multi-specific or universal engagers.

Aspect 142. The effector cell of aspect 127, wherein effector cell is modified to block or decrease the expression of a first endogenous TCR subunit and/or a second endogenous TCR subunit.

Aspect 143. The effector cell of aspect 127 which does not express a T cell receptor (TCR) and/or CD3ε, CD3γ or CD3δ and which is modified by recombinant expression to express a recombinant double chain SAR comprising a non TCR antigen-recognition domain and a T cell receptor module (TCRM), wherein said cell expresses CD3 chains CD3γ, CD36, CD3E and CD3ƒ, and the CD3 chains and the SAR form a functional CD3-SAR complex located at the surface of the cell.

Aspect 144. The effector cell of aspect 127 which does not express a T cell receptor (TCR) and/or does not express CD3ε, CD3γ or CD3δ and which is modified by recombinant expression to express a recombinant double chain TCR exogenous to the cell, wherein said recombinant double chain TCR is a SAR which comprises a TCR antigen-recognition domain comprising a) Vα and Vβ domains or b) Vγ and Vδ domains and a non-T cell receptor module (NTCRM).

Aspect 145. The effector cell of aspect 144, comprising a TCR antigen recognition motif that is operationally linked via optional linkers to a non-T cell receptor module (NTCRM) comprising a first MAM and a second MAM derived from non-T cell receptors and/or signaling adaptors and further comprising optional cytosolic co-stimulatory domains.

Aspect 146. The effector cell of aspects 143-145, which is a selected from the group consisting of NK cell, g-NK cell, memory like NK cells, cytokine induced killer cell (CIK), iPSC, modified HLA deficient iPSC, iPSC-derived NK cell, B cell, a granulocyte, a macrophage/monocyte, a dendritic cell, a T cell that is deficient in one or more of TCRα, TCRβ, TCRγ, TCRδ, CD3γ, CD36, CD3E or CD3 chains, an immortalized cell line, an immortalized NK cell line, NK92 cell line, NK92MI cell line, YTS cells or derivative thereof.

Aspect 147. A method of generating effector cells of aspect 127, comprising introducing in vitro transcribed RNA or RNAs or synthetic RNA or RNAs into a cell or population of cells, where the RNA or RNAs comprises a recombinant polynucleotide or polynucleotides of aspect 118.

Aspect 148. A method of providing anti-disease immunity in a subject comprising administering to the subject an effective amount of the immune effector cell or a stem cell that can give rise to an immune effector cell of any one of aspects 127-146, wherein the cell is an autologous T cell or an allogeneic T cell, or an autologous NK cell or an allogeneic NK cell, or an autologous macrophage or an allogeneic macrophage, or an autologous granulocyte or an allogeneic granulocyte, or an autologous dendritic cell or an allogeneic dendritic cell, or an autologous hematopoietic stem or an allogeneic hematopoietic stem cell or an autologous or an allogeneic iPSC that can give rise to an effector cell.

Aspect 149. The method of aspect 148, wherein the allogeneic T, NK, macrophage, granulocyte, dendritic cell, hematopoietic stem cell or iPSC lacks expression or has low expression of a functional TCR, a functional HLA, 02 macroglobulin, TAP1, TAP2, tapasin, NLRC5, CIITA, RFXANK, CIITA, RFX5, RFXAP, TCRα or R constant region, NKG2A, NKG2D, CD3ε, CD5, CD52, CD33, CD123, CLL-1, CIS, CBL-B, SOCS2, PD1, CTLA4, LAG3, TIM3, TIGIT, or any gene in the chromosome 6p21 region; and/or introduced or increased expression in at least one of HLA-E, 41BBL, CD3ε, CD3γ, CD36, CD3ƒ, FcRγ, DAP10, DAP12, CD4, CD8, CD16, CD47, CD94, CD113, CD131, CD137, CD80, PDL1, A2AR, Fc receptor, an engager, or surface triggering receptor for coupling with bi- or multi-specific or universal engagers.

Aspect 150. A method of killing a target cell presenting a target antigen, comprising contacting the target cell with the effector cell of aspect 127, wherein the SAR specifically binds to the target antigen.

Aspect 151. A method of aspect 150, further comprising contacting the target cells with one or more agents that bind to one or more antigens expressed on the SAR-expressing effector cell and one or more antigens expressed on a target cell.

Aspect 152. A method of aspect 151 wherein the agent can redirect SAR-expressing effector cells to a target cell expressing an antigen targeted by the agent.

Aspect 153. A method of aspect 151, wherein the agent is an antibody, an antibody, antibody, an antigen binding domain, non-Immunoglobulin antigen binding domain fragment, an autonomous antigen binding domain, a bispecific engager, a bispecific T cell engager (BiTE), a bispecific Killer engager (BiKE), a trispecific engager, a trispecific T cell engager, or a trispecific Killer engager (TRiKE) or a combination thereof.

Aspect 154. A method of aspect 151, wherein the effector cell expresses a SAR comprising the extracellular domain of one or more naturally occurring receptors.

Aspect 155. A method of aspect 154, wherein the SAR comprises the extracellular domain of one or more naturally occurring receptor selected from the group of CD16A, CD16B, CD64, CD32, NKp30, NKp44, NKp46, KIR2DL1, KIR2DL2, KIR2DL3, KIR2DL5A, KIR2DL5B, KIR3DL1, KIR3DL2, KIR3DL4, KIR2DL4, KIR2DS1, KIR2DS2, KIR2DS3, KIR2DS4, KIR2DS5, KIR3DS1, NKG2D, NKG2C, NKG2A, NKG2E, NKG2F, DNAM-1, 2B4, OX40, CD28, 4-1BB, CD27, CD81, CD2, CD5, TNFR-I, TNFR-II, Fas, CD30, CD40, CRTAM, TIGIT, CD96, SLAMF6, SLAMF7, CD100, CD160, CEACAM, ILT2, KLRG1, LAIR1 and CD161.

Aspect 156. A method of aspects 151, wherein the agent is an antibody, antibody, an antigen binding domain, non-Immunoglobulin antigen binding domain fragment, an autonomous antigen binding domain, a bispecific engager, a bispecific T cell engager (BiTE), a bispecific Killer engager (BiKE), a trispecific engager, a trispecific T cell engager, or a trispecific Killer engager (TriKE) that comprises at least one domain that can specifically bind to one or more extracellular domains of the naturally occurring receptors or variants or fragments thereof comprising the SAR.

Aspect 157. A method of aspects 151 or 156, wherein the agent specifically binds to: a) the extracellular domains of one or more naturally occurring receptors or variants or fragments thereof comprising a SAR; and/or b) the extracellular domains of one or more naturally occurring receptors that are not part of the SAR.

Aspect 158. A method of aspects 151 or 157, wherein the agent can specifically bind to the extracellular domain of one or more naturally occurring co-stimulatory receptors.

Aspect 159. A method of aspects 151 or 157, wherein the agent can specifically bind to the extracellular domain of one or more naturally occurring activating receptors.

Aspect 160. A method of aspects 151 or 157, wherein the agent can specifically bind to the extracellular domain of a SAR comprising a co-stimulatory domain.

Aspect 161. A method of aspects 151 or 157, wherein the agent can specifically bind to the extracellular domain of a SAR comprising an activation domain and a co-stimulatory domain.

Aspect 162. A method of aspect 154 or 155, wherein the SAR expresses the extracellular domain of an Fc receptor and the agent is an antibody, antibody, an antigen binding domain, non-Immunoglobulin antigen binding domain fragment, an autonomous antigen binding domain, a bispecific engager, a bispecific T cell engager (BiTE), a bispecific Killer engager (BiKE), a trispecific engager, a trispecific T cell engager, or a trispecific Killer engager (TRiKE) that comprises an Fc domain.

Aspect 163. A method of aspect 162, wherein the Fc receptor is one or more of CD16A, CD16B, CD64, CD32 or a variant or a fragment thereof.

Aspect 164. A method of aspect 151, wherein the target antigen is one or more of antigens listed in Table B.

Aspect 165. A method of aspect 148 or 149, wherein the subject is administered an effective amount of an immune effector cell of one of aspects 123-146 comprising a synthetic antigen receptor (SAR) molecule in combination with an agent that modulates the survival, proliferation, differentiation and/or efficacy of the immune cell, wherein the agent is selected from one or more of: a) a protein phosphatase inhibitor; b) a kinase inhibitor; c) a Lck kinase inhibitor; d) agents that bind to one or more antigens expressed on the SAR-expressing effector cell and one or more antigens expressed on a target cell; e) a cytokine; f) an inhibitor of an immune inhibitory molecule; g) an agent that decreases the level or activity of a TREG cell; h) an agent that increase the proliferation and/or persistence of SAR-modified cells; i) a chemokine; j) an agent that increases the expression of SAR; k) an agent that allows regulation of the expression or activity of SAR; 1) an agent that allows control over the survival and/or persistence of SAR-modified cells; m) an agent that controls the side effects of SAR-modified cells; n) a Brd4 inhibitor; o) an agent that delivers a therapeutic or prophylactic agent to the site of the disease; p) an agent that increases the expression of the target antigen against which SAR is directed; q) an agent that binds to a multipurpose switch co-expressed with the SAR; and r) an adenosine A2a receptor antagonist.

Aspect 166. A pharmaceutical composition comprising a SAR polypeptide molecule of aspect 1, a polynucleotide of aspect 118, a vector of aspect 119, a cell of any one of aspects 127-146, and/or an agent of aspect 151 and 165 and a pharmaceutically acceptable carrier.

Aspect 167. A method of preventing or treating a target antigen-associated disease in an individual in need thereof comprising administering to the individual an effective amount of the pharmaceutical composition of aspect 166.

Aspect 168. The method of aspect 167, wherein the target antigen-associated disease is selected from the group consisting of a proliferative disease, a precancerous condition, a cancer, an immune disease, an allergic disease, a degenerative disease, an infectious disease, and a non-cancer related indication.

Aspect 169. The use or method of aspect 168, wherein the cancer is a hematologic cancer chosen from one or more of chronic lymphocytic leukemia (CLL), acute leukemias, acute lymphoid leukemia (ALL), B-cell acute lymphoid leukemia (B-ALL), T-cell acute lymphoid leukemia (T-ALL), chronic myelogenous leukemia (CML), B cell prolymphocytic leukemia, blastic plasmacytoid dendritic cell neoplasm, Burkitt's lymphoma, diffuse large B cell lymphoma, primary effusion lymphoma, follicular lymphoma, hairy cell leukemia, small cell- or a large cell-follicular lymphoma, malignant lymphoproliferative conditions, MALT lymphoma, mantle cell lymphoma, marginal zone lymphoma, primary effusion lymphoma (PEL), multiple myeloma, myelodysplasia and myelodysplastic syndrome, non-Hodgkin's lymphoma, Hodgkin's lymphoma, plasmablastic lymphoma, plasmacytoid dendritic cell neoplasm, Waldenstrom macroglobulinemia, or pre-leukemia.

Aspect 170. The use or method of aspect 168, wherein the cancer is selected from the group consisting of colon cancer, rectal cancer, renal-cell carcinoma, liver cancer, non-small cell carcinoma of the lung, cancer of the small intestine, cancer of the esophagus, melanoma, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin's lymphoma, cancer of the endocrine system, cancer of the thyroid gland, cancer of the parathyroid gland, cancer of the adrenal gland, sarcoma of soft tissue, cancer of the urethra, cancer of the penis, solid tumors of childhood, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, Merkel cell cancer, epidermoid cancer, squamous cell cancer, T-cell lymphoma, environmentally induced cancers, combinations of said cancers, and metastatic lesions of said cancers.

Aspect 171. The use or method of aspect 168, wherein the disease is associated with infection by a virus selected from the group consisting of coronavirus, SARS-CoV2 and variants, HIV1, HIV2, HTLV1, Epstein Barr virus (EBV), cytomegalovirus (CMV), adenovirus, adeno-associated virus, BK virus, Human Herpesvirus 6, Human Herpesvirus 8 influenza virus, parainfluenza virus, avian flu virus, MERS and SARS coronaviruses, Crimean Congo Hemorrhagic fever virus, rhino virus, enterovirus, Dengue virus, West Nile virus, Ebola virus, Marburg virus, Lassa fever virus, zika virus, RSV, measles virus, mumps virus, rhino virus, varicella virus, herpes simplex virus 1 and 2, varicella zoster virus, HIV-1, HTLV1, Hepatitis virus, enterovirus, hepatitis B virus, Hepatitis C virus, Nipah and Rift valley fever viruses, Japanese encephalitis virus, Merkel cell polyomavirus, or is associated with infection with Mycobacterium tuberculosis, atypical mycobacteria species, Pneumocystis jirovecii, toxoplasmosis, rickettsia, nocardia, aspergillus, mucor, or candida.

Aspect 172. The use or method of aspect 168, wherein the disease is an immune or degenerative disease selected from the group consisting of diabetes mellitus, multiple sclerosis, rheumatoid arthritis, pemphigus vulgaris, ankylosing spondylitis, Hoshimoto's thyroiditis, SLE, sarcoidosis, scleroderma, mixed connective tissue disease, graft versus host disease or Alzheimer's disease.

Aspect 173. A method for investigating the transduction efficiency of a vector encoding a SAR and a multipurpose switch of aspect 93 which comprises the step of detecting expression of the multi-purpose switch on the surface of cells transfected or transduced with the vector.

Aspect 174. A method for selecting cells expressing a SAR of aspect 95, which comprises the following steps: i) detecting expression of the multipurpose switch on the surface of cells transfected or transduced with a vector according to aspect 140; and (ii) selecting cells which are identified as expressing the multipurpose switch.

Aspect 175. A method of preparing a purified population of cells enriched for cells expressing a SAR, which comprises the step of selecting cells expressing a SAR from a population of cells using a method according to aspect 174.

Aspect 176. A method of aspect 175, which comprises the following steps: (i) transducing or transfecting a population of cells isolated from a patient ex vivo with a vector according to aspect 140; and (ii) selecting cells expressing the SAR from the transduced/transfected population of cells by a method according to aspect 174.

Aspect 177. A cell population which is enriched for cells expressing a multipurpose switch polypeptide of aspects 94-117, and thus enriched for cells expressing a SAR.

Aspect 178. A method for tracking transduced cells in vivo which comprises the step of detection of expression of a multipurpose switch polypeptide according to any of aspects 173 at the cell surface.

Aspect 179. A method for deleting a cell of aspect 127, which comprises the step of exposing the cells to an agent that binds to the accessory module comprising the multipurpose switch.

Aspect 180. A method of aspect 179, wherein a) the multipurpose switch comprises a sequence with SEQ ID NO: 7843-7850 or a variant with 80% homology thereto and the agent is belantamab mafodotin; b) the multipurpose switch comprises a sequence with SEQ ID NO: 9620-9624 or a variant with 80% homology thereto and the agent is Rituximab or a CD20 antibody; c) the multipurpose switch comprises a sequence with SEQ ID NO: 9625 or a variant with 80% homology thereto and the agent is Herceptin, Enhertu or a Her2 targeted antibody; and d) the multipurpose switch comprises a sequence with SEQ ID NO:7850 or a variant with 80% homology thereto and the agent is Adcetris or a CD30 targeted antibody.

Aspect 181. A kit comprising at least one SAR polypeptide molecule of aspect 1, an accessory module of aspect 93, a multiple purpose switch of aspect 94, a recombinant polynucleotide of aspect 118, a recombinant expression system of aspect 119, a vector of aspect 123 or the cell of aspect 127, an agent of aspect 151 and/or 165 and a composition of aspect 166.

Aspect 182. A method of aspect 140, which is carried out a) ex vivo; b) in vivo; or c) both ex vivo and in vivo.

Aspect 183. A SAR of aspect 1, comprising at least two chains wherein a) a first polypeptide chain comprises a first antigen-binding domain comprising a Vα or a Vγ domain and a first Membrane associated module (MAM); and b) a second polypeptide chain comprises a second antigen-binding domain comprising a Vβ or a Vδ domain and a second Membrane associated module (MAM); wherein the Vα or Vγ domain of the first antigen-binding domain and the complementary Vβ or Vδ domain of the second antigen-binding domain form a TCR-Fv like antigen-binding module that specifically binds to a target antigen; and wherein the first MAM and the second MAM form a non-T cell receptor module (NTCRM) that is capable of activating at least one signaling pathway and/or recruiting at least one signaling adaptor.

Aspect 184. The SAR of aspect 183, wherein the first polypeptide chain further comprises a first peptide linker between the first antigen-binding domain and the first MAM, and the second polypeptide chain further comprises a second peptide linker between the second antigen-binding domain and the second MAM.

Aspect 185. The SAR of aspect 184, wherein the first and/or second peptide linkers comprise, individually, a constant domain or fragment thereof from an immunoglobulin or T cell receptor subunit.

Aspect 186. A SAR of aspect 183, wherein the first polypeptide further comprises a first cytosolic domain C-terminal to the first transmembrane/membrane-anchoring domain comprising the first MAM; and/or wherein the second polypeptide further comprises a second cytosolic domain C-terminal to the second transmembrane/membrane anchoring domain comprising the second MAM.

Aspect 187. The SAR of aspect 183, wherein the first polypeptide chain further comprises a first accessory intracellular domain comprising a co-stimulatory domain sequence C-terminal to the first transmembrane/membrane anchoring domain of the first MAM; and/or wherein the second polypeptide chain further comprises a second accessory intracellular domain comprising a co-stimulatory domain sequence C-terminal to the second transmembrane/membrane anchoring domain comprising the second MAM.

Aspect 188. A SAR of aspect 187, wherein the co-stimulatory domain is selected from CD28, 4-1BB, OX40, 2B4, CD27, CD81, CD2, CD5, BAFF-R, CD30, CD40, HVEM or ICOS, or a variant or a fragment thereof.

Aspect 189. A SAR of aspect 183, wherein the first and/or the second MAM and the NTCRM are comprised of the transmembrane/membrane anchored domain, optional cytosolic domain, optional hinge domain and/or optional extracellular domain of a non-T cell receptor and/or a signaling adaptor.

Aspect 190. A SAR of aspect 189, wherein a) the non T cell receptor is selected from the group consisting of CD16A, CD16B, CD64, CD32, NKp30, NKp44, NKp46, KIR2DL1, KIR2DL2, KIR2DL3, KIR2DL5A, KIR2DL5B, KIR3DL1, KIR3DL2, KIR3DL4, KIR2DL4, KIR2DS1, KIR2DS2, KIR2DS3, KIR2DS4, KIR2DS5, KIR3DS1, NKG2D, NKG2C, NKG2A, NKG2E, NKG2F, DNAM-1, 2B4, OX40, CD28, 4-1BB, CD27, CD81, CD2, CD5, TNFR-I, TNFR-II, Fas, CD30, CD40, CRTAM, TIGIT, CD96, SLAMF6, SLAMF7, CD100, CD160, CEACAM, ILT2, KLRG1, LAIR1, CD161, a variant of any of the foregoing and fragments thereof; and/or b) the signaling adaptor is selected from the group consisting of: CD3ƒ, FcRγ, DAP10, a variant of any of the foregoing and fragments thereof.

Aspect 191. A SAR of aspect 183, which when expressed in a non-T cell confers on it a T cell receptor like target binding recognition and/or recruitment of a at least one signaling adaptor and/or activation of at least one signaling pathway.

Aspect 192. A method of aspect 167, wherein the subject is further administered a therapeutic effective amount of a tyrosine kinase inhibitor to a) prevent or reverse toxicity due to administration of a pharmaceutical composition comprising SAR expressing effector cells; and/or b) prevent or reverse exhaustion of SAR expressing effector cells.

Aspect 193. A method of aspect 192, wherein the wherein the tyrosine kinase inhibitor is a Lck inhibitor.

Aspect 194. The method of aspect 192, wherein the tyrosine kinase inhibitor is dasatinib or ponatinib.

Aspect 195. The method of aspect 192, wherein treatment increases secretion of IL-2 by T cells in the subject.

Aspect 196. The method of aspect 192, wherein treatment decreases apoptosis of T cells in the subject.

Aspect 197. The method of aspect 192, wherein treatment decreases expression of at least one T cell exhaustion marker selected from the group consisting of PD-1, TIM-3, and LAG-3.

Aspect 198. The method of aspect 192, wherein treatment increases expression of CD62L or CCR7.

Aspect 199. The method of aspect 192, wherein multiple cycles of treatment are administered to the subject.

Aspect 200. The method of aspect 192, wherein the tyrosine kinase inhibitor is administered intermittently.

Aspect 201. The method of aspect 192, wherein the tyrosine kinase inhibitor is administered for a period of time sufficient to restore at least partial T cell function then discontinued.

Aspect 202. The method of aspect 192, wherein the tyrosine kinase inhibitor is administered orally.

Aspect 203. The method of aspect 192, wherein the toxicity related to genetically engineered T cell administered to a subject is cytokine release syndrome.

Aspect 204. The method of aspect 192, wherein the toxicity related to genetically engineered T cell administered to a subject is on-target off tumor toxicity or off-target off-tumor toxicity.

Aspect 205. The method of aspect 192, wherein the subject is human.

Aspect 206. A cell that is not a T cell with target recognition properties and function of a T cell, wherein the cell a) lacks the expression of one or all TCR constant chains or a fragment thereof selected from the group of TCRα, TCRβ, TCRγ, TCRδ or preTCR; and/or b) lacks the expression of one or more of CD3 chains selected from the group of CD3ε, CD3γ and/or CD36; and/or c) lacks the ability to form a functional TCR module (TCRM).

Aspect 207. A cell of aspect 206 which expresses a double chain receptor that comprises a TCRM and confers on the cell target recognition properties of a T cell.

Aspect 208. A cell of aspect 207, that is capable of expressing on cell surface a receptor that can form a TCR-Fv antigen binding module that specifically binds to a target antigen.

Aspect 209. A receptor of aspect 208, where the two variable domains comprising the TCR-Fv are not part of a single polypeptide chain.

Aspect 210. A cell of aspect 206, where the two variable domains comprising the TCR-Fv are a) Vα and VD, or b) Vγ and V6.

Aspect 211. A method of killing a target cell presenting a target antigen, comprising contacting the target cell with the effector cell of aspect 206, wherein the cell specifically recognizes the target antigen.

Aspect 212. A cell of aspect 210, which can kill a target cell expressing its target peptide antigen.

Aspect 213. A pharmaceutical composition comprising a cell of aspect 206 and a pharmaceutically acceptable carrier.

Aspect 214. A method of preventing or treating a target antigen-associated disease in an individual in need thereof comprising administering to the individual an effective amount of a cell of aspect 206 or the pharmaceutical composition of aspect 213.

Aspect 215. A method of making a non-T cell of aspect 206 with T cell receptor like antigen recognition.

Aspect 216. A method of aspect 215, wherein a non-T cell with TCR like antigen recognition does not express a) TCRα, TCRβ, TCRγ, TCRδ and preTCRα chains, or b) A dimer of TCRα and TCRβ chains, or c) A dimer of TCRγ and TCRS chains, or d) A dimer of preTCRα and TCRβ chains.

Aspect 217. A method of aspect 215, wherein the method does not involve a) exogenous expression of a TCR chain, or b) Exogenous expression of a CD3 chain selected from the group of CD3ε, CD3γ and CD36.

Aspect 218. A method of aspect 215, wherein the method involves a single genetic modification.

Aspect 219. A method of aspect 215, wherein the method involves introduction of one or two recombinant polynucleotides encoding a double chain receptor.

Aspect 220. A cell of aspect 210, which is a NK cell, iNKT (innate natural killer cell), g-NK cell, memory like NK cells, cytokine induced killer cell (CIK), iPSC, a modified HLA deficient iPSC, iPSC-derived NK cell, B cell, a macrophage/monocyte, granulocyte, a dendritic cell, an immortalized cell line, an immortalized NK cell line, NK92 cell line, NK92MI cell line, YTS cell, NKG cell line or a derivative thereof.

Aspect 221. An isolated fusion protein between a Type II transmembrane protein and a Type I transmembrane protein or a secreted protein with an N-terminal signal peptide.

Aspect 222. An isolated fusion protein of aspect 221, comprising the cytosolic, transmembrane and partial or entire extracellular domain of a Type II protein in fusion with the extracellular domain of a type I transmembrane protein or a secreted protein with an N-terminal signal peptide.

Aspect 223. An isolated fusion protein of aspect 221, where the N-terminus of a polypeptide encoding the entire or partial extracellular domain of the type I membrane protein or the secreted protein with an N-terminal signal peptide is operationally linked to the C-terminus of the Type II protein in N-terminus to C-terminus orientation.

Aspect 224. A method of making a fusion protein of aspect 221 comprising the steps of a) fusing in frame the 5′ end of a polynucleotide encoding the type I membrane protein or a secreted protein with an N-terminal signal peptide to the 3′ end of a nucleotide encoding the partial or entire extracellular domain of the type II protein; and b) introducing the recombinant polynucleotide in suitable cell so as to allow the expression of the fusion protein.

Aspect 225. An isolated fusion protein of aspect 221, where the fusion protein encodes for a chimeric antigen receptor or a synthetic antigen receptor targeting a specific antigen.

Aspect 226. A pharmaceutical composition comprising a cell made by aspect 224, expressing a fusion protein of aspect 221, and a pharmaceutically acceptable carrier.

Aspect 227. A method of treatment using a composition of aspect 226.

Aspect 228. A recombinant polynucleotide encoding a synthetic immune receptor comprising a sequence selected from the group consisting of SEQ ID NO:1600-2328, 4851-5129, 5451-6282, 7160-7170, 7601-7747, 8768-9602, 10817-10830 or a sequence with at least 75% identity to a nucleotide sequence encoding a synthetic immune receptor set forth in any one of the above.

Aspect 229. An amino acid sequence encoding a synthetic immune receptor polypeptide selected from the group consisting of SEQ ID NO:3994-4722, 5151-5429, 6283-7114, 7852-7862, 8293-8439, 9860-10694 or a sequence with at least 75% identity to an amino acid sequence encoding a synthetic immune receptor set forth in any one of the above.

### Examples

Cell lines engineered to express luciferases (e.g., GLuc or NLuc) for measuring cytotoxicity of different constructs targeting different cell surface and intracellular antigens are provided in Table A. Cell lines used in these experiments, target antigens on the cells lines and their growth media are shown in the following Table A. Cells were cultured at 37° C., in a 5% CO₂ humidified incubator. The cell lines were obtained from ATCC, NIH AIDS reagent program or were available in the laboratory.

TABLE A

| Cell line | Culture Conditions | Exemplary CAR Target Antigens Expressed |
|---|---|---|
| BC-1 | RPMI, 20% FCS | BCMA, GPRC, CD138 |
| BC-3 | RPMI, 20% FCS | BCMA, GPRC, CD138 |
| BCBL-1 | RPMI, 20% FCS | GPRC, CD138 |
| JSC-1 | RPMI, 20% FCS | GPRC, CD138 |
| MM1S | RPMI, 10% FCS | CD38, GPRC, CD44, CD200R |
| U266 | RPMI, 10% FCS | BCMA, WT1/HLA-A2+, CS1, CLL1, CD138, c-MET, |
| | | IL6R, CD179b, NY-ESO1/HLA-A2, NYBR, LAMP1 |
| L363 | RPMI, 10% FCS | BCMA, GPRC, WT1/HLA-A2+, CS1, CLL1, CD138, NY- |
| | | ESO1/HLA-A2, NYBR, LAMP1 |
| K562 | RPMI, 10% FCS | CD33, IL1Ra, TnAg |
| BV173 | RPMI, 10% FCS | CD123, CD179b, IL1Ra, WT1/HLA-A2+, CXCR4, FLT3, |
| | | CD179a |
| Nalm6 | RPMI, 10% FCS | CD19, CD20, CD22, CD179b, CD179a |
| HL60 | RPMI, 10% FCS | CD33, CD34, CLL1, IL6R, CD32, CD179 |
| U937 | RPMI, 10% FCS | CD4, CLL1 |
| RS:411 | RPMI, 20% FCS | CD19, Folate Receptor beta (FRbeta), TGFbeta, CD179b, |
| | | NKG2D, FLT3, CD179a |
| MV:411 | RPMI, 10% FCS | FLT3, CD123, FRbeta |
| Raji | RPMI, 10% FCS | CD19, CD20, CD22, BCMA, CD38, CD70, CD79, CLL1 |
| HEL-92.1.7 | RPMI, 10% FCS | MPL, CD33, CD32, CD200R |
| Jurkat | RPMI, 10% FCS | TnAg, TSLRP, TSHR, CD4, CD38 |
| Daudi | RPMI, 10% FCS | BCMA, FRbeta |
| REC-1 | RPMI, 10% FCS | NKG2D, ROR1 |
| KG-1 | RPMI, 20% FCS | CD33, CD34, CD123, TSLRP |
| CEM | RPMI, 10% FCS | CD5, CD43 |
| U937 | RPMI, 10% FCS | CD4, CLL1 |
| LAMA5 | RPMI, 10% FCS | WT1/HLA-A2 |
| A549 | DMEM, 10% FCS | ROR1, CD22, TIM1, CDH17 |
| HT29 | DMEM, 10% FCS | EGFR, SLEA, c-MET |
| Molm-13 | RPMI, 20% FCS | FLT3, IL6R, LAMP1, TSLRP, CD4, CSF2RA, CXCR4, |
| | | IL6R, CSF2RA, GPC3 |
| A431 | DMEM, 10% FCS | EGFR, Folate Receptor Alpha, Her3 |
| P19 | DMEM, 10% FCS | SSEA |
| THP-1 | RPMI, 10% FCS | CD32, CD33, CXCR4, CD123, CD44, IL6R, Folate |
| | | Receptor beta, CD70, LAMP1, FLT3, CSF2RA |
| U87MG | DMEM, 10% FCS | CD276, gpNMB, IL13RA2 |
| LoVo | DMEM, 10% FCS | Tissue Factor, CDH17, EGFR |
| SKOV-3 | DMEM, 10% FCS | FR1a, FSHR, Her2, Her3, LHR, MSLN, TIM1, EPCAM |
| NCI-H1993 | DMEM, 10% FCS | EGFR |
| Kasumi-1 | RPMI, 20% FCS | CLEC5A, PR1/HLA-A2, TGFbeta, |
| Jeko-1 | RPMI, 20% FCS | BCMA, ROR1 |
| PC-3 | DMEM, 10% FCS | CGH, TROP2, PSCA, PSMA. EPCAM, FSHR, CLD18.2A2 |
| HeLa | DMEM, 10% FCS | EGFR, FR1, MSLN, TSHR |
| LnCap | DMEM, 10% FCS | EGFR, FSHR, PSCA, PSMA, CD22, Her3, CD22, LHR, |
| OVCAR-3 | DMEM, 10% FCS | B7H4, CDH6, DLL3, FR1, FSH, LHR, MSLN, PTK7, |
| | | TnAg, TSHR, L1CAM |
| MEL-624 | DMEM, 10% FCS | CDH19, GD2, GD3, gp100/HLA-A2, gpNMB, HMWMAA, |
| | | NY-ESO1/HLA-A2, MART1/HLA-A2 |
| LS174-T | DMEM, 10% FCS | CEA |
| MEL-526 | DMEM, 10% FCS | GD2 |
| MDA- | DMEM, 10% FCS | CD324, Muc1 |
| MB231 | | |
| L1236 | RPMI, 20% FCS | CD30, CD23, PDL1 |
| L428 | RPMI, 20% FCS | CD30, CD123, CCR4, PDL1 |
| L540 | RPMI, 20% FCS | CD30, CCR4, PDL1 |
| Molt-16 | RPMI, 20% FCS | IL1ra, NKG2D |
| CEM | RPMI, 10% FCS | CD5 |
| MG-63 | DMEM, 10% FCS | IL13RA2 |
| Karpass-299 | RPMI, 20% FCS | Alk, GPRC, PDL1 |
| MCF7 | DMEM, 10% FCS | B7D4, CD276, TROP2, Her3, Muc1, LewisY, LHR |
| AA-2 | RPMI, 10% FCS | HIV1 env glycoprotein (gp120) |
| HL2/3 | DMEM, 10% FCS | HIV1 env glycoprotein (gp120) |
| TF228.1.16 | DMEM, 10% FCS | HIV1 env glycoprotein (gp120), CCR4 |
| TT | DMEM, 10% FCS | TGF-Beta, TSHR, GFRalpha4 |
| DMS79 | RPMI, 10% FCS | Fucosyl-GM1, Slea (CA19.9; Sialyl Lewis Antigen) |
| LAN-5 | DMEM, 10% FCS | ALK, DLL3, GFRalpha4, FUCOSYL-GM1 |
| PEER1 | RPMI, 10% FCS | TSHR |
| SK-MEL-37 | DMEM, 10% FCS | DLL3, GD2 |
| F9 | DMEM, 10% FCS | SSEA |
| HepG2 | DMEM, 10% FBS | GPC3, AFP/HLA-A2 |

Jurkat cell line (clone E6-1) engineered with a NFAT-dependent EGFP (or GFP) reporter gene and named JNG was a gift from Dr. Arthur Weiss at University of California San Francisco and have been described to study CAR-signaling ((Wu, C Y et al., Science 350:293-302,2015). Jurkat cells were maintained in RPMI-1640 medium supplemented with 1000 FBS. NK92MI cells were obtained from ATCC and were maintained as per the instructions provided. NK92 cells were also obtained from ATCC and maintained in RPMI medium with 20% o FBS and 200 U/mL of hIL2. T2 cells were from ATCC.

### Generation of Lentiviral Vectors Encoding SARs

The SAR constructs were cloned in the lentiviral, retroviral or sleeping beauty transposon vectors. Exemplary vectors are provided in SEQ ID NO: 1-6. Other vectors that can be used for generating SARs of the disclosure are known in the art. The psPAX2 vector was a gift from Didier Trono (Addgene plasmid #12260). The pLP/VSVG envelope plasmid and 293FT cells were obtained from Invitrogen (ThermoFisher Scientific). The retroviral transfer vector MSCVneo, MSCVhygro, and MSCVpac and the packaging vector pKAT have been described previously (PCT/US2018/53247). The methods for generation of SAR (e.g., 2^{nd} generation CARs, SIRs, Ab-TCR and TFP etc.), the generation and use of GGS-NLuc fusion proteins, and the generation and use of luciferase (e.g., GLuc and Luc146-1H2) reporter cell lines for measurement of cellular cytotoxicity using the Matador assays have been described (PCT/US2017/024843, PCT/US2017/025602, PCT/US2017/052344, PCT/US2017/064379 and PCT/US2018/53247), which are incorporated in their entirety by reference herein.

The sequences comprising the antigen binding domains of SAR are codon optimized and synthesized artificially using publically available software (e.g. ThermoFisher or IDT) and commercial vendors (e.g. IDT). The resulting fragments are PCR amplified and cloned in different vectors containing the different SIR backbones using standard Molecular Biology techniques. In general SAR constructs are typically cloned in a lentiviral vector. The sequences of the SIR constructs are confirmed using automated sequencing.

An exemplary SAR construct encoding vector is pCCLc-MNDU3-Nhe-CD8SP-R1-NY-ESO-IG4-Vb-Xho-TCROECD-Bam-CD3zECDTMCP-opt-F-P2A-Spe-SP-Bst-NY-ESO-IG4-Va-Mlu-TCRuECD-Kpn-CD3zECDTMCP-opt2-F-F2A-Xba-PAC-Sal-AWPRE (SEQ ID NO: 9366). This construct is cloned in the pCCLc-MNDU3-delta-WPRE lentiviral vector backbone (SEQ ID NO: 6). The vector comprises an MNDU3 promoter that drives the expression of a SAR construct comprising a nucleotide comprising a CD8 signal peptide (SEQ ID NO: 31), an EcoR I site, the Vβ/Vb domain of a TCR (IG4) targeting NY-ESO-1 (SEQ ID NO: 966), Xho I site, a TCRPECD linker (or TCR0-Ig3; SEQ ID NO: 1166), BamH I restriction site, a CD3zECDTMCP-opt signaling chain (SEQ ID NO: 1089) comprising the extracellular, transmembrane and cytoplasmic domain of human CD3z, a Furine cleavage site, a P2A cleavable linker, an Spe I restriction site, a signal peptide, a Bst I restriction site, Va/Va domain of a TCR (IG4) targeting NY-ESO-1 peptide/HLA-A2 (SEQ ID NO: 966), aMlu I site, a TCRUECD linker (or TCRa-Ig3; SEQ ID NO: 1168), a Kpn I restriction site, a second CD3zECDTMCP-opt2 signaling module (SEQ ID NO: 10816) comprising the extracellular, transmembrane and cytoplasmic domain of human CD3z, a Furine cleavage site, a F2A cleavable linker, an Xba I restriction site, a puromoycin resistance (PAC) cassette and a Sal I restriction site. The expression cassette has many convenient restriction sites so that the different modules comprising antigen binding domain fragments (e.g., Vb, Va, vL and vH domains), linkers (e.g., TCROECD, TCRUECD, IgCL or IgG-CHI), or signaling chains (e.g., CD3zECDTMCP-opt and opt2) can be cut out and replaced with the different modules. Thus, a person with ordinary skills in the art can use this vector and the sequence of the antigen binding domain (e.g., vL and vH domains of an antibody) to generate a SAR targeting any other new antigen and comprising different linkers and signaling chains.

### Lentivirus and Retrovirus Vectors

Lentiviruses were generated in 293FT cells by transfection of transfer plasmids encoding the different SAR constructs and 2^{nd} or 3^{rd} generation packaging plasmids using polyethylene amine (PEI) essentially as described previously (Natarajan et al, Scientific Reports, 10:2318) and PCT/US2018/53247. 293FT cells were grown in DMEM with 10% FCS (hereby referred to as DMEM-10). Approximately 48-72 hrs post-transfection, all media was collected, pooled and centrifuged at 1000 rpm for 1 minute to remove any cell debris and non-adherent cells. The cell-free supernatant was filtered through 0.45 μm syringe filter. In some cases, the supernatant was further concentrated by centrifugation at 18500 rpm for 2 hours at 4° C. The viral pellet was re-suspended in 1/10 of the initial volume in XVIVO medium. The virus was either used fresh to infect the target cells or stored frozen in aliquots at −80° C.

### Infection of T Cells, NK Cells and PBMC

Buffy coat cells were obtained from healthy de-identified adult donors from the Blood Bank at Children Hospital of Los Angeles and used to isolate peripheral blood mononuclear cells (PBMC) by Ficoll-Hypaque gradient centrifugation. PBMC were either used as such or used to isolate T cells or NK cells using magnetic microbeads (Miltenyi Biotech) and following the manufacturer's instructions. PBMC or isolated T cells were re-suspended in XVIVO medium (Lonza) supplanted with 10 ng/ml CD3 antibody, 10 ng/ml CD28 antibody and 100 IU recombinant human-IL2. Cells were cultured at 37° C., in a 5% C02 humidified incubator. Cells were activated in the above medium for 1 day prior to infection with lentiviral vectors. In general, primary cells (e.g., T cells) were infected in the morning using spin-infection (1800 rpm for 90 minutes at 37° C. with 300 μl of concentrated virus that had been re-suspended in XVIVO medium in the presence of 8 μg/ml of Polybrene® (Sigma, Catalog no. H9268). The media was changed in the evening and the infection was repeated for two more days for a total of 3 infections. After the 3rd infection, the cells were pelleted and resuspended in fresh XVIVO media containing 10 ng/ml CD3 antibody, 10 ng/ml CD28 antibody and 100 IU recombinant human-IL2 and supplemented with respective antibiotics (if indicated) and place in the cell culture flask for selection, unless indicated otherwise. Cells were cultured in the above medium for 10-15 days in case no drug selection was used and for 20-30 days in case drug-selection was used. For infection of JNG and cancer cell lines, approximately 500,000 cells were infected with 2 ml of the un-concentrated viral supernatant in a total volume of 3 ml with Polybrene® (Sigma, Catalog no. H9268). Then next morning, the cells were pelleted and resuspended in the media with respective antibiotics, where appropriate, and place in the cell culture flask for selection. Primary NK cells were stimulated with NK activation beads (Miltenyi Biotech) for 24-96 h prior to infection with concentrated lentiviral vectors without Polybrene®. Cells were expanded in IL-2 comprising NK medium before analysis.

Blood from a healthy donor was used to isolate NK cells using NK cell isolation kit (Miltenyi). NK92 cells were obtained from ATCC. NK Primary and NK92 cells were cultured in Minimum Essential Medium (MEM) Alpha without ribonucleosides and deoxy ribonucleosides supplemented with 20% Fetal bovine serum, 0.2 mM Inositol, 0.1 mM 2-Mercaptoethanol, 2 mM L-Glutamine, 1.5 g/L Sodium bicarbonate, 0.02 mM Folic Acid. For NK92 cells, medium was further supplemented with 200 IU/ml IL2. NK primary cells were cultured and activated with 500 IL/ml of IL2 for 7 days before infections. Lentiviral infections were done with concentrated lentivirus supernatant by spin infection in 6-well plates. For primary NK cells, approx. 4 million NK cells in 1.5 ml culture medium supplemented with 500 IU/ml IL2 and infected with 500p concentrated virus without polybrene. For NK92 cells, 6μg/ml polybrene was used. The plates were centrifuged at 2,800 rpm for 90 min at 32° C. for 5 hours. The medium was changed after 5 hours, and the infection was repeated next day.

Essentially a similar procedure as described above for lentivirus vector production was used for generation of retroviral vectors with the exception that 293FT cells were generally transfected in 10 cm tissue culture plates in 10 ml of DMEM-10 medium using 10 μg of retroviral construct, 4pg of pKAT and 2pg of VSVG plasmid. The virus collection and infection of target cells was carried out essentially as described above for lentiviral vectors.

### Antibodies, Peptides and Drugs

NY-ESO1 (SEQ ID NO:10880), MAGE-A3-270-270 (SEQ ID NO: 10878) and MAGE-A3-112-120 (SEQ ID NO: 10879) peptides were synthesized by Genscript. Digitonin was purchased from Sigma (Cat. no D141) and a stock solution of 100 mg/ml was made in DMSO. A diluted stock of 1 mg/ml was made in PBS. Final concentration of digitonin used for cell lysis was 30pg/ml unless indicated otherwise.

### ELISA

Human IL2, IFNγ, IL6 and TNFα were measured in the cell culture supernatant of CAR-expressing Jurkat-NFAT-GFP effector cells or T cells that had been co-cultured with the specific target cell lines for 24 to 96 hours using commercially available ELISA kits from R&D systems (Minneapolis, MN) and BD Biosciences and following the recommendations of the manufacturer.

### FACS Analysis for Detecting Expression of SAR

Mouse Anti-Human c-Myc APC-conjugated Monoclonal Antibody (Catalog #IC3696A) was from R&D Systems (Minneapolis, MN). Biotinylated protein L was purchased from GeneScript (Piscataway, NJ), reconstituted in phosphate buffered saline (PBS) at 1 mg/ml and stored at 4° C. Streptavidin-APC (SA1005) was purchased from ThermoFisher Scientific. APC-labelled NY-ESO-1/HLA-A2 and MAGE-A3 (270-279)-HLA-A2 tetramers were obtained from NIH tetramer facility at Emory University. They target NY-ESO1 (SEQ ID NO:10880) and MAGE-A3-270-279 (SEQ ID NO: 10878) peptides.

For detection of SARs using tetramer, 1×10⁶ cells were harvested and washed three times with 3 ml of ice-cold 1×PBS containing 4% bovine serum albumin (BSA) wash buffer. After wash, cells were resuspended in 0.1 ml of the ice-cold wash buffer containing 10 μl of APC-conjugated tetramer and incubated in dark for 1 hour followed by two washings with ice cold wash buffer before analysis by FACS.

For detection of SARs using Protein L staining, 1×10⁶ cells were harvested and washed three times with 3 ml of ice-cold 1×PBS containing 4% bovine serum albumin (BSA) wash buffer. After wash, cells were resuspended in 0.1 ml of the ice-cold wash buffer containing 1 μg of protein L at 4° C. for 1 hour. Cells were washed three times with ice-cold wash buffer, and then incubated (in the dark) with 10p of APC-conjugated streptavidin in 0.1 ml of the wash buffer for 30 minutes followed by two washings with ice cold wash buffer. FACS was done using FACSVerse analyzer from BD Biosciences.

### Cell Death Assay

To measure cell death, Matador assay based on ectopic cytosolic expression of Gluc, NLuc or thermostable beetle luciferase (LucPPe or Luc146-1H2) was utilized as described in PCT/US2017/052344 “A Non-Radioactive Cytotoxicity Assay”. Unless indicated otherwise, the target cells stably expressing the different luciferases were plated in triplicate in a 384 well plate in the media used for growing the target cells. Target cells which grow in suspension were generally plated at a concentration of 2-3×10⁴ per well, while target cells which grow as adherent monolayers were plated at a concentration of 1-2×10⁴ per well. Unless indicated otherwise, the target cells were cocultured with the genetically modified (i.e., expressing SAR) effector cells (e.g., T and NK cells or cell lines (NK92, NK92MI or THP cells) at an Effector: Target (E:T) ratio varying from 1:1 to 10:1 for 4 hours to 96 hours. In the case target cells grow as adherent cells (e.g., HeLa cells), they were allowed to attach to the bottom of the wells overnight before the T cells were added. In the case of GLuc expressing target cells, effector cell mediated induction of lysis of target cells was assayed by increase of luciferase activity as measured by BioTek synergy plate reader by directly injecting 0.5×CTZ assay buffer containing native coeloentrazine (Nanaolight). D-luciferin was used as a substrate for target cells expressing Luc146-1H2. Luciferase activity in wells containing media alone (Med) and in wells in which target cells were incubated with effector cells that were not infected (UI) with any SAR construct were used as controls, where indicated.

### Assay to Detect the Expression of Antigens on Target Cells and to Determine the Antigen Binding Activity of Various of Antigen Binding Moieties Used in the Construction of the SARs

The expression of antigens on target cells was determined by bioinformatics in combination with immunostaining with antibodies or a highly sensitive antigen detection assay as described in PCT/US2017/025602 and incorporated herein in its entirety by reference.

### The Immune Effector Cells Expressing SAR are Tested in the Following Assays to Identify the Functional SAR.

(A) Topanga Assay (NLuc binding assay): The control vector- and SAR-expressing Jurkat-NFAT-GFP, T cells or NK cells are stained with the target CD19-Nluc fusion protein and their ability to bind to the target antigen is assayed by measuring Nluc activity using the Topanga Assay. As shown in the following Figure, NK92MI cells expressing the novel next generation SAR (SEQ ID NO: 2275) in which the vL and vH fragments of a CD19 monoclonal hu-mROO5-1 are attached to the hinge, transmembrane and cytosolic domains of DAP10 via IgCL and IgG-CH1 linkers show increased binding to CD19-Nluc Topanga reagent as compared to control parental cells. The 2^{nd} generation CAR (SEQ ID NO: 5141) serves as a positive control.

TABLE 50

| | Average | SD |
|---|---|---|
| Parental | 16 | 2 |
| CD8SP-CD19-hu-mROO5-1-vL- IgCL-DAP10-opt1-F- | 2235 | 94 |
| P2A-Spe-IgSP- CD19-hu-mROO5-1-vH-IgG-CH1-DAP10- | | |
| opt2-F-F2A- PAC (SEQ ID NO: 2275) | | |
| CD8SP-FMC63-scFv-Myc-BBz-T2A-PAC (SEQ ID NO: | 4842 | 63 |
| 5141) | | |

The experiment is repeated with NK92MI cells expressing different next generation SARs. As shown in the following FIG., NK92MI cells expressing the SAR (SEQ ID NO: 2277) showed very high binding to CD19-Nluc fusion protein in the Topanga Assay. NK92MI cells expressing DAP10-SAR (SEQ ID NO: 2275) also showed modest CD 19-binding.

TABLE 51

| | Average | SD |
|---|---|---|
| Parental | 108 | 7 |
| SEQ ID NO: 2275 | 648 | 189 |
| SEQ ID NO: 2278 | 145 | 2.8284 |
| SEQ ID NO: 2277 | 49100 | 5081 |
| SEQ ID NO: 5141 | 1274 | 349 |

Assay for Cytotoxic Activity in vitro. The uninfected NK92 or T cells or those expressing a control vector or SAR are cocultured with the target cell lines expressing a non-secretory form of a luciferase (such as GLuc, NLuc, Turboluc 16 etc.) for 4-96 hours and induction of cell lysis examined by measuring the luciferase activity as described in PCT/US17/52344. As shown in the following Table, NK92MI cells expressing the SAR (SEQ ID NO: 2277) showed highest cytotoxicity as measured by Matador Assay. NK92MI cells expressing DAP10-SAR (SEQ ID NO: 2275) also showed modest cytotoxicity.

TABLE 52

| RAJI-MATADOR ASSAY | Average | SD |
|---|---|---|
| Medium | 9308 | 634 |
| Parental | 39075 | 3842 |
| SEQ ID NO: 2278 | 39424 | 366 |
| SEQ ID NO: 2275 | 53450 | 2739 |
| SEQ ID NO: 2277 | 89496 | 2318 |
| SEQ ID NO: 5141 | 37447 | 981 |

### NF-κB Activation Assay

Jurkat cells were engineered to express firefly luciferase cDNA (Luc) under NF-κB responsive promoter. The cells were subsequently infected with lentiviral vectors encoding the following SAR constructs to generate cells stably expressing the different SARs. The Jurkat-NF-κB cells expressing the different SARs were cocultured with RAJI-wt (CD19+ve) or RAJI-CD19-KO (CD19-null) cells for 24 hours and luciferase activity was measured.

TABLE 53

| | Average | | | Std Dev | | |
|---|---|---|---|---|---|---|
| | | Raji- | | | Raji- | |
| | | CD19- | | | CD19- | |
| SAR NAME | alone | KO | Raji | alone | KO | Raji |
| SEQ ID NO: 2275 | 448 | 681 | 991 | 79 | 113 | 91 |
| (SEQ ID NO: 2277) | 670 | 995 | 1602 | 137 | 118 | 224 |
| SEQ ID NO: 1860 | 572 | 1337 | 843 | 20 | 88 | 647 |
| SEQ ID NO: 5141 | 572 | 853 | 2104 | 74 | 4 | 952 |

The accompanying Table 53 shows induction of NF-κB activity by Jurkat cells expressing the SARs with SEQ ID NO: 2275 and 2277. There was no NF-κB induction by NKp46 SAR with SEQ ID NO: 1860.

Induction of NFAT promoter driven GFP expression. Jurkat-NFAT-GFP (JNG) cells are infected with lentiviral vectors encoding different SAR constructs and selected with puromycin. The control JNG cells and SAR-expressing JNG cells are cocultured for approximately 24 hours with different target cell lines expressing their cognate antigen(s). Thus, JNG cells expressing SARs targeting CD19 are co-cultured with CD19 antigen-expressing cell line RAJI and their ability to bind to the target antigen and induce cell signaling is assayed by measuring induction of GFP expression using Flow Cytometry. RAJI cells lacking CD19 (RAJI-CD19-KO) are used as negative controls. The induction of GFP expression is quantified as 1+, 2+, 3+ etc. depending on % of SAR expressing cells that induce GFP over the control cells. Thus, 1.6+ means that approximately 16% of JNG cells expressing the SAR showed GFP induction upon coculture with the target cells above the level seen with control JNG parental cells. The results in the following Table demonstrate activation of NFAT induced GFP expression upon expression of most single chain and double chain SAR constructs. The results also show induction of NFAT by double chain heterodimeric SAR constructs, such as SARs represented by SEQ ID NO: 2276, 2280, 2314-2316, 2319-2326. The results further show that the SAR constructs expressing at least one chain with full extracellular domain of CD16A not only respond to cells expressing the antigen(s) targeted by their antigen binding domain (e.g., BCMA-FHVH) but also retain the ability to bind to Fc region of an antibody targeting a different antigen. Thus, in an exemplary embodiment, JNG cells expressing the SAR construct CD8SP-Sph-BCMA-FHVH93-Kpn-G4S-EcoR1-CD16A-F158V-FL-F-P2A-Spe-IgHSP-Apa-CD20-VHH-USC1-2HC2D6-Bam-G4S-Bst-CD16A-F158V-FL-v2-F2A-Xba-PAC(SEQ ID NO: 2283) induces NFAT-driven GFP when co-cultured with RAJI (CD19+ and BCMA+) and L363 (CD19-ve/BCMA+) cell lines via its antigen binding domains BCMA-FHVH93 and CD20-VHH-USC1-2HC2D6, respectively. More importantly, JNG cells expressing this SAR (SEQ ID NO: 2283) construct fail to induce GFP when co-cultured with Her2-expressing SKOV3 cells. However, strong induction of GFP is observed when the JNG cells expressing this SAR are co-cultured with SKOV3 cells in the presence of Herceptin (1pg/ml) that can bind to either of the two CD16A-F158V-FL chains comprising this SAR construct. Essentially similar results are obtained when the experiment is repeated with JNG cells expressing the SAR construct CD8SP-Sph-BCMA-FHVH93-Kpn-G4S-EcoR1-CD16A-V158A-FL-F-P2A-Spe-IgHSP-Apa-CD20-VHH-USC1-2HC2D6-Bam-G4S-Bst-CD19-hu-mROO5-1-vH-Mlu-[hTCRa-T48C-opt]-F-F2A-Xba-PAC (SEQ ID NO: 2314). This construct is a double chain heterodimeric SAR in which one signaling chain comprises of CD16A-V158A-FL and the other signaling chain comprises of [hTCRa-T48C-opt]. In contrast, the SAR constructs represented by SEQ ID NO: 2315 and 2316 that comprise a CD16A chain (CD16-V158A-D2TMCP-v1) lacking the first Ig like domain fail to induce GFP when co-cultured with SKOV3 cells in the presence of Herceptin. However, these SAR constructs can still activate GFP upon co-culture with RAJI cells, suggesting functional signaling by the CD20-VHH-USC1-2HC2D6-Bam-G4S-Bst-CD19-hu-mROO5-1-vH-Mlu-[hTCRa-T48C-opt] chain present in this construct. On the other hand, the JNG cells expressing this SAR fail to induce GFP upon co-culture with L363 cells suggesting that the BCMA-FHVH93-Kpn-CD16-V158A-D2TMCP chain is not expressed or is not functionally active. Similarly, JNG cells expressing the construct CD8SP-Sph-BCMA-FHVH93-Kpn-CD16-V158A-D2TMCP-v1-F-P2A-Spe-IgHSP-Apa-CD20-VHH-USC1-2HC2D6-Bam-CD16-F158V-S197P-D2TMCP-v3-F-F2A-Xba-PAC (SEQ ID NO: 2322) fail to induce GFP when co-cultured with RAJI or L363 cells. Further, these cells fail to induce GFP when co-cultured with SKOV3 cells in the presence of Herceptin. These results suggest that neither BCMA-FHVH93-Kpn-CD16-V158A-D2TMCP-v1 nor CD20-VHH-USC1-2HC2D6-Bam-CD16-F158V-S197P-D2TMCP-v3 chains is functionally expressed.

The results further demonstrate that JNG cells expressing a number of double chain constructs comprising vL and vH fragments attached to two separate chains are capable of inducing GFP upon co-culture with their cognate antigen expressing cells. Exemplary such constructs are represented by 2275-2278, 2280-2282, 2319, 2321, 2300, 2323-2326. These results demonstrate that vL and vH fragments can assemble to form a functional Fv capable of binding to the cognate antigen and transmitting a signal even when they are attached to two separate chains. This is observed when the two chains are structurally distinct and not known to hetero or homo-dimerize. Exemplary such double chain heterodimeric constructs are represented by SEQ ID NO: 2276, 2280, 2323-2326.

The results further demonstrate that SAR constructs comprising one or two chains encoding the entire extracellular, transmembrane and cytosolic domain of CD16 (e.g., SEQ ID NO:3843, 3853 and 3863) show strong induction of NFAT-driven GFP as compared to SAR constructs encoding CD16 chains that are missing the first Ig domain of CD16 (e.g., SEQ ID NO: 3844, 3854, and 3864). Further, SARs constructs comprising CD16 one or two chains encoding the entire extracellular, transmembrane and cytosolic domain of CD16 (e.g., SEQ ID NO:3843, 3853 and 3863) in general show stronger induction of NFAT-driven GFP. Similarly, JNG cells expressing the SAR constructs (e.g., SEQ ID NO: 2297) comprising the entire extracellular, transmembrane and cytosolic domain of NKp30 (e.g., NKp30-ECDTMCP-opt1 or opt2) represented by SEQ ID NO: 3763 or 3769 show strong induction of NFAT-driven GFP when co-cultured with their cognate antigen expressing cell lines.

TABLE 54

| SEQ | | NFAT-GFP |
|---|---|---|
| ID NO | NAME OF THE SIR CONSTRUCT | ASSAY |
| 2275 | CD8SP-CD19-hu-mROO5-1-vL-IgCL-DAP10-opt1-F-P2A-IgSP- | RAJI (+1.5), |
| | CD19-hu-mROO5-1-vH-Ig1CH1-DAP10-opt2-F-F2A-PAC | RAJI−CD19− |
| | | KO (−) |
| 2276 | CD8SP-hCD19-EUK-5-13-vL-IgCL-CD3zECDTMCP-opt-F-P2A- | RAJI (+1.3) |
| | SP-hCD19-EUK-5-13-vH-IgG1-CH1-CD16A-Hinge-TM-CP- | |
| | F158V-F-P2A-PAC | |
| 2277 | CD8SP-hCD19-EUK-5-13-vL-IgCL-CD3zECDTMCP-opt-F-P2A- | RAJI (+1.5) |
| | SP-hCD19-EUK-5-13-vH-IgG1-CH1-CD3zECDTMCP-opt2-F- | |
| | F2A-PAC | |
| 2279 | CD8SP-CD19-hu-mROO5-1-CD8-hing-NKG2D-TM-2B4z-F- | RAJI (+2.5), |
| | P2A-CD3z-P2A-SynthK13-Flag-F-P3A-PAC | RAJI−CD19− |
| | | KO (−) |
| 2280 | CD8SP-CD19-hu-mROO5-1-vL-IgCL-NC-D2GKN-ECD-TM-CP- | RAJI (+0.6) |
| | opt1-F-P2A-CD19-hu-mROO5-1-vH-Hinge-TM-CP-NKp46-opt2- | |
| | F-F2A-PAC | |
| 2281 | CD8SP-CD19-hu-mROO5-1-vL-NKp46-Hinge-TM-CP-opt1-F- | RAJI (+1.4) |
| | P2A-IgSP-CD19-hu-mROO5-1-vH-NKp46-Hinge-TM-CP-opt2-F- | |
| | F2A-PAC | |
| 2283 | CD8SP-BCMA-FHVH93-G4S-CD16A-F158V-FL-F-P2A-IgHSP- | RAJI (+4.2), |
| | CD20-VHH-USC1-2HC2D6-G4S-CD16A-F158V-FL-v2-F2A- | L363 (+1.4), |
| | PAC | SKOV3 (−), |
| | | SKOV3 + |
| | | Herceptin (+7.1) |
| 2314 | CD8SP-BCMA-FHVH93-G4S-CD16A-V158A-FL-F-P2A-IgHSP- | RAJI (+2.9), |
| | CD20-VHH-USC1-2HC2D6-G4S-CD19-hu-mROO5-1-vH- | L363 (+1.1), |
| | [hTCRa-T48C-opt]-F-F2A-PAC | SKOV3 (−), |
| | | SKOV3 + |
| | | Herceptin (+6.5) |
| 2315 | CD8SP-BCMA-FHVH93-G4S-CD16A-V158A-D2TMCP-v1-F- | RAJI (+2.8), |
| | P2A-IgHSP-CD20-VHH-USC1-2HC2D6-G4S-CD19-hu-mR005- | L363 (+/−), |
| | 1-vH-[hTCRa-T48C-opt]-F-F2A-PAC | SKOV3 (−), |
| | | SKOV3 + |
| | | Herceptin (−) |
| 2316 | CD8SP-BCMA-FHVH93-CD16-V158A-D2TMCP-v1-F-P2A- | RAJI (+4.4), |
| | IgHSP-CD20-VHH-USC1-2HC2D6-G4S-CD19-hu-mROO5-1-vH- | L363 (−), |
| | [hTCRa-T48C-opt]-F-F2A-PAC | SKOV3 (−), |
| | | SKOV3 + |
| | | Herceptin (−) |
| 2317 | CD8SP-BCMA-FHVH93-G4S-CD16-V158A-D2TMCP-v1-F- | RAJI (−), |
| | P2A-PAC | L363 (−) |
| 2318 | SP-CD20-VHH-USC1-2HC2D6-G4S-CD16A-F158V-FL-v2-F2A- | RAJI (+3.5) |
| | PAC | |
| 1638 | CD8SP-BCMA-FHVH93-G4S-CD16A-V158-FL-F-P2A-PAC- | L363 (+2) |
| 2319 | CD8SP-CD19-hu-mROO5-1-vL-NKp30-ECDTMCP-opt1-F-P2A- | RAJI (+0.8) |
| | IgSP-CD19-hu-mROO5-1-vH-NKp46-Hinge-TM-CP-opt2-F-F2A- | |
| | PAC | |
| | CD8SP-NKp30-Ig-Hinge-opt1-G4S-humROO5-vL-[hTCRb- | RAJI (+1.3) |
| | S57C]-F-P2A-IgHSP-CD20-VHH-USC1-2HC2D6-G4S-CD19-hu- | |
| | mROO5-1-vH-[hTCRa-T48C-opt]-F-F2A-PAC | |
| | CD8SP-NKp44-Ig-opt1-[hTCRb-S57C]-F-P2A-IgHSP-CD20- | RAJI (+0.8) |
| | VHH-USC1-2HC2D6-G4S-CD19-hu-mROO5-1-vH-[hTCRa- | |
| | T48C-opt]-F-F2A-PAC | |
| | CD8SP-NKp44-Ig-opt1-G4S-humROO5-vL-[hTCRb-S57C]-F- | RAJI (+0.5) |
| | P2A-IgHSP-CD20-VHH-USC1-2HC2D6-G4S-CD19-hu-mROO5- | |
| | 1-vH-[hTCRa-T48C-opt]-F-F2A-PAC | |
| 2320 | CD8SP-BCMA-FHVH93-G4S-NKp44-Hinge-TMCP-opt1-F-P2A- | RAJI (+1.1), |
| | IgHSP-CD20-VHH-USC1-2HC2D6-G4S-CD16A-v158-FL-v2- | L363 (+/−), |
| | F2A-PAC | SKOV3 (−), |
| | | SKOV3 + |
| | | Herceptin (+6.2) |
| 2321 | CD8-hCD19-EUK-5-13-vL-IgCL-NKp44-ECDTMCP-opt1-F- | RAJI (+0.3) |
| | P2A-SP-hCD19-EUK-5-13-vH-IgG1-CH1-CD3zECDTMCP-opt2- | |
| | F-F2A-PAC | |
| | CD19-hu-mROO5-1-vL-NKp44-ECDTMCP-opt1-F-P2A-IgSP- | RAJI (+0.5) |
| | CD19-hu-mROO5-1-vH-NKp46-Hinge-TM-CP-opt2-F-F2A-PAC- | |
| | (100620-BBW1) | |
| 2300 | CD8SP-CD19-hu-mROO5-1-vL-NKp44-Hinge-TMCP-opt1-F- | RAJI (+0.4) |
| | P2A-IgSP-CD19-hu-mROO5-1-vH-NKp44-Hinge-TMCP-opt2-F- | |
| | F2A-PAC | |
| | CD8SP-hCD19-EUK-5-13-vL-IgCL-mutCD3z-ECDTM-2B4CP- | RAJI (+0.2) |
| | opt1-CD3zCP-opt1-F-P2A-dSPE-IgSP-hCD19-EUK-5-13-vH- | |
| | IgG1-CH1-DAP10-opt2-CD3zCP-opt2-F-F2A-PAC | |
| 2322 | CD8SP-BCMA-FHVH93-CD16-V158A-D2TMCP-v1-F-P2A- | RAJI (+/−), |
| | IgHSP-CD20-VHH-USC1-2HC2D6-CD16-F158V-S197P- | L363 (−), |
| | D2TMCP-v3-F-F2A-PAC | SKOV3 (−), |
| | | SKOV3 + |
| | | Herceptin (−) |
| 2323 | CD8-hCD19-EUK-5-13-vL-IgCL-NKp30-Hinge-TMCP-opt1-F- | RAJI (+0.4) |
| | P2A-SP-hCD19-EUK-5-13-vH-IgG1-CHI-CD3zECDTMCP-opt2- | |
| | F-F2A-PAC | |
| 2325 | CD8SP-NKp30-Ig-Hinge-opt1-[hTCRb-S57C]-F-P2A-IgHSP- | RAJI (+0.6) |
| | CD20-VHH-USC1-2HC2D6-G4S-CD19-hu-mROO5-1-vH- | |
| | [hTCRa-T48C-opt]-F-F2A-PAC | |
| 2324 | CD8-hCD19-EUK-5-13-vL-IgCL-NKp44-Hinge-TMCP-opt1-F- | RAJI (+0.4) |
| | P2A-SP-hCD19-EUK-5-13-vH-IgG1-CH1-CD3zECDTMCP-opt2- | |
| | F-F2A-PAC | |
| 2326 | hCD19-EUK-5-13-vL-IgCL-CD8-hinge-NKG2D-TM-2B4-CP-opt- | RAJI (+0.4) |
| | 1-CD3zCP-opt1-F-P2A-dSPE-IgSP-hCD19-EUK-5-13-vH-IgG1- | |
| | CH1-DAP10-opt2-CD3zCP-opt2-F-F2A-dXba-PAC | |
| 2293 | CD8SP-CD19-hu-mROO5-1-vL-NKp30-ECDTMCP-opt1-F-P2A- | RAJI (+0.3) |
| | SP-CD19-hu-mROO5-1-vH-NKp30-ECDTMCP-opt2-F-F2A-PAC | |
| 2294 | CD8SP-NKp30-Ig-Hinge-opt1-G4S-humROO5-vL-[hTCRb- | RAJI (+1.2) |
| | S57C]-F-P2A-IgHSP-NKp30-Ig-Hinge-opt2-G4S-CD19-hu- | |
| | mROO5-1-vH-[hTCRa-T48C-opt]-F-F2A-PAC | |
| 2296 | CD8SP-BCMA-FHVH93-G4S-NKp44-Hinge-ECDTMCP-opt1-F- | RAJI (+0.3), |
| | P2A-IgHSP-CD20-VHH-USC1-2HC2D6-G4S-NKp44-Hinge- | L363 (−) |
| | ECDTMCP-opt2-F2A-PAC | |
| 2297 | CD8SP-BCMA-FHVH93-G4S-NKp30-ECDTMCP-opt1-F-P2A- | RAJI (+1.2), |
| | IgHSP-CD20-VHH-USC1-2HC2D6-G4S-NKp30-ECDTMCP- | L363 (+1.1) |
| | opt2-F-F2A-PAC | |
| 2300 | CD8SP-CD19-hu-mROO5-1-vL-NKp44-Hinge-TMCP-opt1-F- | RAJI (+0.4) |
| | P2A-IgSP-CD19-hu-mROO5-1-vH-NKp44-Hinge-TMCP-opt2-F- | |
| | F2A-PAC | |
| 2299 | CD8SP-CD19-hu-mROO5-1-vL-NKp44-ECDTMCP-opt1-F-P2A-IgSP-CD19-hu- | |
| | mROO5-1-vH-NKp44-ECDTMCP-opt2-F-F2A-PAC | |
| 2292 | CD8SP-hCD19-EUK-5-13-vL-IgCL-NKp30-ECDTMCP-opt1-F- | RAJI (−) |
| | P2A-SP-hCD19-EUK-5-13-vH-IgG1-CH1-NKp30-ECDTMCP- | |
| | opt2-F-F2A-PAC | |
| 2298 | CD8-hCD19-EUK-5-13-vL-IgCL-NKp44-ECDTMCP-opt1-F- | RAJI (−) |
| | P2A-SP-hCD19-EUK-5-13-vH-IgG1-CH1-NKp44-ECDTMCP- | |
| | opt2-F-F2A-PAC | |
| 2299 | CD8-hCD19-EUK-5-13-vL-IgCL-NKp30-ECDTMCP-opt1-F- | RAJI (+1.1) |
| | P2A-SP-hCD19-EUK-5-13-vH-IgG1-CH1-CD3zECDTMCP-opt2- | |
| | F-F2A-PAC | |
| 2299 | CD8-hCD19-EUK-5-13-vL-IgCL-Xho-NKp30-ECDTMCP-opt1-F-P2A- | RAJI +1.1 |
| | Spe-SP-Bst-hCD19-EUK-5-13-vH-IgG1-CH1-Mlu-CD3zECDTMCP- | |
| | opt2-F-F2A-PAC | |

A number of double chain SIR with deleted TCRα, β, γ and δ chains were constructed and expressed in JNG cells. As shown in the following Table, surprisingly, SIRs with deleted TCRα, β, γ or δ chains (SEQ ID NO: 7619-7625 and showed strong NFAT-GFP activity when expressed in JNG cells. Thus, deleted TCRα, β, γ or δ chains can be used to generate a diverse panel of SIR with varying expression and signaling activity to generate a diverse immune response.

TABLE 55

| SEQ ID | | JURKAT NFAT- |
|---|---|---|
| NO (DNA) | Name of fragment | GFP ASSAY |
| 7601 | CD8SP-BCMA-FHVH93-G4S-CD16A-V158-FL-F-P2A-PAC | L363 +2 |
| 7602 | CD20-Ubli-NKp44-ECDTMCP-opt2-F-F2A-PAC | |
| 7603 | BCMA-J6M0-NKp44-ECDTMCP-opt2-F-F2A-PAC | L363 +1 |
| 7604 | CD22-h10F4v2-NKp44-ECDTMCP-opt2-F-F2A-PAC | RAJI +0.5 |
| 7605 | 8SP-BCMA917-vHH-E59D-NKp44-ECDTMCP-opt2F2A-PAC | L363 +1 |
| 7606 | CD8SP-PSMA-USC76-chVH-NKp44-ECDTMCP-opt2-F-F2A- | LnCAP +0.5 |
| | PAC | |
| 7607 | CD8SP-CD19-hu-mROO5-1-scFv-NKp30-ECDTMCP-opt2-F- | RAJI +3.5 |
| | F2A-PAC | |
| 7608 | FMC64-NKp30-ECDTMCP-opt2-F-F2A-PAC | RAJI +2.5 |
| 7609 | CD20-2F2-NKp30-ECDTMCP-opt2-F-F2A-PAC | RAJI +2 |
| 7610 | CD8SP-Hu161-2-NKp30-ECDTMCP-opt2-F-F2A-PAC | HEL +0.5 |
| 7611 | BCMA-J6M0-NKp30-ECDTMCP-opt2-F-F2A-PAC | L363 +1 |
| 7612 | CD22-h10F4v2-NKp30-ECDTMCP-opt2-F-F2A-PA | RAJI +0.5 |
| 7613 | CD8SP-hu-HA22-1-NKp30-ECDTMCP-opt2-F-F2A-PAC | RAJI +1 |
| 7614 | CD8SP-CD20-VHH-USC1-2HC2D6-NKp30-ECDTMCP-opt2-F- | RAJI +2 |
| | F2A-PAC | |
| 7615 | CD8SP-CD38-331-vHH-D64E-NKp30-ECDTMCP-opt2-F-F2A- | L363 +1 |
| | PAC | |
| 7616 | CD8SP-BCMA917-vHH-E59D-NKp30-ECDTMCP-opt2-F-F2A- | L363 +1 |
| | PAC | |
| 7617 | CD8SP-PSMA-USC76-chVH-NKp30-ECDTMCP-opt2-F-F2A- | LnCAP +1.5 |
| | PAC | |
| 7618 | CD8SP-BCMA-FHVH93-G4S-NKp46-opt1-F-P2A::Xba-PAC | L363 +0.5 |
| 7619 | CD19-hu-mROO5-1-vL-huTCRg-F-P2A-SP-CD19-hu-mROO5- | RAJI +1.5 |
| | 1-vH-huTCRd-d2-F-F2A-PAC | |
| 7620 | CD19-hu-mROO5-1-vL-huTCRg-d1-F-P2A-SP-CD19-hu- | RAJI +3 |
| | mROO5-1-vH-huTCRd-d2-F-F2A-PAC | |
| 7621 | CD19-hu-mROO5-1-vL-huTCRg-d2-F-P2A-SP-CD19-hu- | RAJI +3.5 |
| | mROO5-1-vH-huTCRd-d2-F-F2A-PAC | |
| 7622 | CD19-hu-mROO5-1-vL-huTCRg-d8-F-P2A-SP-CD19-hu- | RAJI +2 |
| | mROO5-1-vH-huTCRd-d2-F-F2A-PAC | |
| 7623 | CD19-hu-mROO5-1-vL-huTCRg-d11-F-P2A-CD19-hu-mROO5- | RAJI +1 |
| | 1-vH-huTCRd-d2-F-F2A-PAC | |
| 7624 | CD19-hu-mROO5-1-vL-huTCRg-d16-F-P2A-CD19-hu-mROO5- | RAJI +1 |
| | 1-vH-huTCRd-d2-F-F2A-PAC | |
| 7625 | CD19-hu-mROO5-1-vL-huTCRg-d21-F-P2A-CD19-hu-mROO5- | RAJI +1 |
| | 1-vH-huTCRd-d2-F-F2A-PAC | |
| 7626 | CD8SP-BCMA-FHVH93-G4S-CD16A-V158-FL-F-P2A-IgHSP- | RAJI+, |
| | CD20-VHH-USC1-2HCD26-GS4-KIR2DL1-ECDTMCP-opt2-F- | L363+ |
| 7627 | CD8SP-BCMA-FHVH93-G4S-CD16A-V158-FL-F-P2A-IgHSP- | RAJI 0.5+, |
| | CD20-VHH-USC1-2HCD26-GS4-OX40-ECDTMCP-opt2-F- | L363 1.5+ |
| | F2A-PAC | |
| 7628 | CD19-hu-mROO5-1-vL-huTCRg-d3-F-P2A-SP-CD19-hu- | RAJI +5 |
| | mROO5-1-vH-huTCRd-d2-F-F2A-PAC | |
| 7629 | CD19-hu-mROO5-1-vL-huTCRg-d4-F-P2A-SP-CD19-hu- | RAJI +4.5 |
| | mROO5-1-vH-huTCRd-d2-F-F2A-PAC | |
| 7630 | CD19-hu-mROO5-1-vL-huTCRg-d31-F-P2A-SP-CD19-hu- | RAJI +1.5 |
| | mROO5-1-vH-huTCRd-d2-F-F2A-PAC | |
| 7631 | CD19-hu-mROO5-1-vL-huTCRg-d9-F-P2A-SP-CD19-hu- | RAJI +1.5 |
| | mROO5-1-vH-huTCRd-d2-F-F2A-PAC | |
| 7632 | CD19-hu-mROO5-1-vL-huTCRg-d26-F-P2A-SP-CD19-hu- | RAJI +2.5 |
| | mROO5-1-vH-huTCRd-d2-F-F2A-PAC | |
| 7633 | CD8SP-BCMA-FHVH93-G4S-CD16A-V158-FL-F-P2A-IgHSP- | RAJI 0.5+, |
| | CD20-VHH-USC1-2HCD26-GS4-CD32-ECDTMCP-opt2-F- | L363 1.0+ |
| | F2A-PAC | |
| 7634 | CD19-hu-mROO5-1-vL-huTCRg-d5-F-P2A-SP-CD19-hu- | RAJI+ |
| | mROO5-1-vH-huTCRd-d4-F-F2A-PAC | |
| 7635 | CD19-hu-mROO5-1-vL-huTCRg-F-P2A-SP-CD19-hu-mROO5- | RAJI 2.5+ |
| 7636 | CD8SP-CD19-hu-mROO5-1-scFv-G3S-NKp46-ECDTMCP-opt2- | RAJI+/− |
| | F-F2A-PAC | |
| 7637 | CD8SP-CD19-hu-mROO5-1-scFv-G3S-CD3e-ECDTMCP-d9- | RAJI 1.5+ |
| | opt2-F-F2A-PAC | |
| 7638 | CD8SP-CD19-hu-mROO5-1-scFv-G3S-CD3e-ECDTMCP-d18- | RAJI −ve |
| | opt2-F-F2A-PAC | |
| 7639 | CD8SP-hCD19-EUK-5-13-vL-IgCL-NKp30-Hinge-TMCP-opt1- | RAJI +0.4 |
| | F-P2A-SP-hCD19-EUK-5-13-vH-IgG1-CH1-CD3zECDTMCP- | |
| | opt2-F-F2A-PAC | |
| 7640 | CD8SP-NKp30-Ig-Hinge-opt1-[hTCRb-S57C]-F-P2A-IgHSP- | RAJI +0.6 |
| | CD20-VHH-USC1-2HC2D6-G4S-CD19-hu-mROO5-1-vH- | |
| | [hTCRa-T48C-opt]-F-F2A-PAC | |
| 7641 | CD8SP-hCD19-EUK-5-13-vL-IgCL-NKp44-Hinge-TMCP-opt1- | RAJI +0.4 |
| | F-P2A-SP-hCD19-EUK-5-13-vH-IgG1-CH1-CD3zECDTMCP- | |
| | opt2-F-F2A-PAC | |
| 7642 | CD8-hCD19-EUK-5-13-vL-IgCL-CD3zECDTMCP-opt-F-P2A- | RAJI +0.1 |
| | SP-hCD19-EUK-5-13-vH-IgG1-CH1-CD16A-v158-S197P-FL- | |
| | v3-F-F2A-PAC | |
| 7643 | CD8SP-CD19-hu-mROO5-vL-IgCL-CD3zECDTMCP-opt-F- | RAJI +0.8 |
| | P2A-SP-CD19-hu-mROO5-vH-IgGA1-CHI-CD3zECDTMCP- | |
| | opt2-F-F2A-PAC | |
| 7644 | CD8SP-CD19-hu-mROO5-vL-IgCL-CD3zECDTMCP-opt-F- | RAJI +1.1 |
| | P2A-SP-CD19-hu-mROO5-vH-IgG2-1C-CHI-CD3zECDTMCP- | |
| | opt2-F-F2A-PAC | |
| 7645 | CD8SP-CD19-hu-mROO5-vL-IgCL-CD3zECDTMCP-opt-F- | RAJI +0.9 |
| | P2A-SP-CD19-hu-mROO5-vH-IgD-CHI-CD3zECDTMCP-opt2- | |
| | F-F2A-PAC | |
| 7646 | CD8SP-CD19-hu-mROO5-vL-IgCL-CD3zECDTMCP-opt-F- | RAJI +1 |
| | P2A-SP-CD19-hu-mROO5-vH-IgG2-0C-CD3zECDTMCP-opt2- | |
| | F-F2A-PAC | |
| 7647 | CD8SP-CD19-hu-mROO5-vL-IgCL-CD3zECDTMCP-opt-F- | RAJI +1.3 |
| | P2A-SP-CD19-hu-mROO5-vH-IgGA2-CHI-CD3zECDTMCP- | |
| | opt2-F-F2A-PAC | |
| 7648 | CD8SP-CD19-hu-mROO5-vL-IgCL-CD3zECDTMCP-opt-F- | RAJI +1.1 |
| | P2A-SP-CD19-hu-mROO5-vH-IgE-CHI-CD3zECDTMCP-opt2- | |
| | F-F2A-PAC | |
| 7649 | CD8SP-CD19-hu-mROO5-vL-IgCL-CD3zECDTMCP-opt-F- | RAJI +3.1; |
| | P2A-SP-CD19-hu-mROO5-vH-IgG4-CHI-CD3zECDTMCP-opt2- | with Dasatinib |
| | F-F2A-PAC | (+2.3) |
| 7650 | CD8SP-CD19-hu-mROO5-vL-IgCL-CD3zECDTMCP-opt-F- | RAJI +0.7 |
| | P2A-SP-CD19-hu-mROO5-vH-IgG3-CHI-CD3zECDTMCP-opt2- | |
| | F-F2A-PAC | |
| 7651 | CD8-hCD19-EUK-5-13-vL-IgCL-CD8-hinge-NKG2D-TM-2B4- | RAJI +0.4 |
| | CP-opt-1-CD3zCP-opt1-F-P2A-IgSP-hCD19-EUK-5-13-vH- | |
| | IgG1-CH1-DAP10-opt2-CD3zCP-opt2-F-F2A-PAC | |
| 7652 | CD8SP-CD19-hu-mROO5-vL-IgCL-CD3zECDTMCP-opt-F- | RAJI +1.7 |
| | P2A-SP-CD19-hu-mROO5-vH-IgM-CHI-CD3zECDTMCP-opt2- | |
| | F-F2A-PAC | |
| 7653 | CD19-hu-mROO5-1-vL-NKp30-ECDTMCP-opt1-F-P2A-IgSP- | RAJI +0.3 |
| | CD19-hu-mROO5-1-vH-NKp30-ECDTMCP-opt2-F-F2A-PAC | |
| 7654 | CD8SP-NKp30-Ig-Hinge-opt1-G4S-humROO5-vL-[hTCRb- | RAJI +1.2, |
| | S57C]-F-P2A-IgHSP-NKp30-Ig-Hinge-opt2-G4S-CD19-hu- | Hela +1.1, |
| | mROO5-1-vH-[hTCRa-T48C-opt]-F-F2A-PAC | K562 +2.5 |
| 7655 | CD8SP-NKp44-Ig-opt1-[hTCRb-S57C]-F-P2A-IgHSP-NKp30-Ig- | K562 +0.1 |
| | Hinge-opt2-[hTCRa-T48C-opt]-F-F2A-PAC | |
| 7656 | CD8SP-BCMA-FHVH93-G4S-NKp44-Hinge-ECDTMCP-opt1- | RAJI +0.3 |
| | F-P2A-IgHSP-CD20-VHH-USC1-2HCD26-G4S-NKp44-Hinge- | |
| | ECDTMCP-opt2-F2A-PAC | |
| 7657 | CD8SP-BCMA-FHVH93-G4S-NKp30-ECDTMCP-opt1-F-P2A- | RAJI +1.7, |
| | IgHSP-CD20-VHH-USC1-2HC2D6-G4S-NKp30-ECDTMCP- | RAJI + 100 nM |
| | opt2-F-F2A-PAC | Dasatinib +0.8; |
| | | L363 +1 |
| 7658 | CD19-hu-mROO5-1-vL-NKp44-Hinge-TMCP-opt1-F-P2A-IgSP- | RAJI +0.3, |
| | CD19-hu-mROO5-1-vH-NKp44-Hinge-TMCP-opt2-F-F2A-PAC | RAJI + 100 nM |
| | | Dasatinib −ve |
| 7659 | CD19-hu-mROO5-1-vL-NKp44-ECDTMCP-opt1-F-P2A-IgSP- | RAJI +1 |
| | CD19-hu-mROO5-1-vH-NKp44-ECDTMCP-opt2-F-F2A-PAC | |
| 7660 | FMC64-CD16A-v158-S197P-FL-v3-F-F2A-PAC | RAJI +0.5 |
| 7661 | CD20-2F2-CD16A-v158-S197P-FL-v3-F-F2A-PAC | RAJI +0.6 |
| 7662 | CD20-Ubli-CD16A-v158-S197P-FL-v3-F-F2A-PAC | RAJI +0.1 |
| 7663 | CD8SP-Hu161-2-CD16A-v158-S197P-FL-v3-F-F2A-PAC | HEL +0.2 |
| 7664 | BCMA-J6M0-CD16A-v158-S197P-FL-v3-F-F2A-PAC | L363 +0.8 |
| 7665 | CD8SP-SC22-HA22-CD16A-v158-S197P-FL-v3-F-F2A-PAC | RAJI +1.5 |
| 7666 | CD22-h10F4v2-CD16A-v158-S197P-FL-v3-F-F2A-PAC | RAJI +0.6 |
| 7667 | CD8SP-hu-HA22-1-CD16A-v158-S197P-FL-v3-F-F2A-PAC | RAJI +1.5 |
| 7668 | hCD19-Bu12-CD16A-v158-S197P-FL-v3-F-F2A-PAC | RAJI +0.3 |
| 7669 | CD8SP-CD20-VHH-USC1-2HC2D6-CD16A-v158-S197P-FL-v3- | RAJI +3.2, |
| | F-F2A-PAC | SKOV3 −ve, |
| | | SKOV3 + |
| | | Herceptin +5.8 |
| 7670 | CD8SP-CD38-331-vHH-D64E-CD16A-v158-S197P-FL-v3-F- | L363 +0.7 |
| | F2A-PAC | |
| 7671 | CD8SP-CD38-331-vHH-S53E-CD16A-v158-S197P-FL-v3-F- | Self-activation |
| | F2A-PAC | |
| 7672 | CD8SP-BCMA917-vHH-E59D-CD16A-v158-S197P-FL-v3-F- | L363 +0.2 |
| | F2A-PAC | |
| 7673 | CD8SP-SARScov2-CR3022-CD16A-v158-S197P-FL-v3-F-F2A- | |
| | PAC | |
| 7674 | CD8SP-CD19-hu-mROO5-vL-IgCL-Hinge-CD16A-v158-S197P- | RAJI +1.7, |
| | FL-v3-F-F2A-SP-CD19-hu-mROO5-vH-IgG1-CH1- | RAJI + 100 nM |
| | CD3zECDTMCP-opt2-F-F2A-PAC | Dasatinib +0.8; |
| | | SKOV3 +0.3, |
| | | SKOV3 plus |
| | | Herceptin 0.2 |
| 7675 | CD8-hCD19-EUK-5-13-vL-IgCL-mutCD3z-ECDTM-2B4CP- | RAJI +2 |
| | opt1-CD3zCP-opt1-F-P2A-IgSP-hCD19-EUK-5-13-vH-IgG1- | |
| | CH1-mutCD3z-ECDTM-2B4CP-opt2-CD3zCP-opt2-F-F2A-PAC | |
| 7676 | CD8SP-CD19-hu-mROO5-1-CD16A-v158-S197P-FL-v3-F-F2A- | RAJI +3 |
| | PAC | |
| 7677 | IgHSP-CD20-VHH-USC1-2HCD26-G4S-CD16A-v158-FL-v2- | RAJI +3.5 |
| | F2A-PAC | |
| 7678 | CD8SP-BCMA-FHVH93-G4S-CD16A-V158-FL-F-P2A::Xba- | L363 +2 |
| | PAC | |
| 7679 | CD8SP-CD19-hu-mROO5-1-scFv-CD16A-v158-S197P-FL-v3 | RAJI +2 |

Induction of NFAT promoter driven GFP expression. Jurkat-NFAT-GFP (TNG) cells are infected with lentiviral vectors encoding different CD16-SAR constructs comprising scFv or vHH domains as the antigen binding domains and CD16A-F158V-S 197P-FL-v3 as the signaling chain. The cells are expanded for 4 days without drug selection. The control JNG cells and SAR-expressing JNG cells are cocultured for approximately 24 hours with different target cell lines expressing their cognate antigen(s). Thus, JNG cells expressing SARs targeting CD19 are co-cultured with CD19 antigen-expressing cell line RAJI and their ability to bind to the target antigen and induce cell signaling is assayed by measuring induction of GFP expression using Flow Cytometry. The results show induction of NFAT-driven GFP expression by JNG cells expressing different SARs when co-cultured with cell lines expressing their target antigen. Essentially similar results are obtained when the experiment is repeated with other CD16 SAR constructs listed in Tables 36-38 of provisional application.

TABLE 56

| | SEQ ID | | NFAT-GFP |
|---|---|---|---|
| TARGET | NO | NAME OF THE SAR CONSTRUCT | ASSAY |
| CD19 | | FMC64-Mlu-CD16A-v158-S197P-FL-v3 | RAJI +0.5 |
| CD20 | 4861 | CD8SP-CD20-2F2-scFv-CD16A-F158V-S197P- | RAJI +0.2 |
| | | FL-v3 | |
| BCMA | 4854 | CD8SP-BCMA-J6M0-scFv-CD16A-F158V- | L363 +0.2 |
| | | S197P-FL-v3 | |
| CD22 | 4983 | CD8SP-CD22-HA22-scFv-CD16A-F158V-S197P- | RAJI +0.1 |
| | | FL-v3 | |
| CD22 | 4863 | CD8SP-CD22-h10F4v2-scFv-CD16A-F158V- | RAJI +0.1 |
| | | S197P-FL-v3 | |
| CD19 | 4858 | CD8SP-CD19Bu12-scFv-CD16A-F158V-S197P- | RAJI +0.3 |
| | | FL-v3 | |
| CD20 | 5047 | CD8SP-CD20-VHH-USC1-CD16A-F158V-S197P- | RAJI +2.6 |
| | | FL-v3 | |

JNG and NK92 cells expressing the different SAR constructs were generated and tested for NFAG-GFP assay and cytotoxicity assay (Matador assay) using the cell lines expressing their target antigen as described in the preceding sections. A summary of the results of different SAR constructs represented by with their SEQ ID NO and target antigen is provided below. Fold induction in the Matador assay was calculated as increase in luciferase activity upon co-culture of target cells with the SAR-expressing NK92 cells as compared to the luciferase activity observed upon co-culture with control NK92 cells. Co-culture assay was conducted for 2 h at E:T ratios between 0.3:1 to 1:1.

TABLE 57

| SEQ ID | | | NK92- Matador |
|---|---|---|---|
| NO (DNA) | TARGET | JNG Assay (NFAT-GFP) | Assay |
| 9245 | CD19 | RAJI(−), RS4−11(−) | RS411(+4.5) |
| 9246 | CD19 | L363(−), U266(−), HepG2(−) | |
| 9247 | CD19 | RAJI(+1) | |
| 9248 | CD19 | RAJI(+1) | |
| 9249 | CD19 | RAJI(−) | RS411(+1.1) |
| 9250 | CD19 | RAJI(+2) | |
| 9251 | CD19 | RAJI(+3) | |
| 9252 | CD19 | RAJI(+/−) | |
| 9253 | CD19 | RAJI(+1) | |
| 9254 | CD19 | RAJI(+/−) | RS411(+2.3) |
| 9255 | CD19 | RAJI2+ | |
| 9256 | CD19 | RAJI(+/−) | |
| 9257 | CD19 | RAJI(+1) | |
| 9258 | CD19 | RAJI(+1.5) | RS411(+26.1) |
| 9259 | CD19 | RAJI(+2) | RS411(+45.1) |
| 9260 | CD19 | RAJI(+2.5) | RS411(+28.8) |
| 9261 | CD19 | RAJI(+2) | RS411(+24.2) |
| 9262 | CD19 | RAJI(+1.5) | ND |
| 9263 | CD19 | RAJI(+1) | ND |
| 9264 | CD19 | RAJI(+/−) | ND |
| 9265 | CD19 | RAJI(+1) | ND |
| 9266 | CD19 | RAJI(+1) | ND |
| 9267 | CD19 | RAJI(+/−) | ND |
| 9268 | CD19 | RAJI(+1) | ND |
| 9269 | CD19 | RAJI(+1) | |
| 9270 | CD19 | RAJI(+1) | RS411(+11.2) |
| 9271 | CD19 | RAJI(+/−) | ND |
| 9272 | CD19 | RAJI(+/−) | RS411(+2.3) |
| 9273 | CD19 | RAJI(+/−) | |
| 9274 | CD19 | RAJI(+/−) | |
| 9275 | CD19 | RAJI(+/−) | RS411(+1.5) |
| 9276 | CD19 | RAJI(+/−) | ND |
| 9277 | CD19 | RAJI(+1) | ND |
| 9278 | CD19 | RAJI(+1) | RS411(+3.4) |
| 9279 | BCMA, | L363(+1.5), U266(+1), LNCaP(+/−) | ND |
| | PSMA | | |
| 9280 | BCMA, | L363(+1.5), U266(+1), LNCaP(+/−) | ND |
| | PSMA | | |
| 9281 | BCMA, | L363(+1.5), U266(+1), LNCaP(−) | ND |
| | PSMA | | |
| 9282 | BCMA, | L363(+1.5), U266(+1.5), LNCaP(−) | ND |
| | PSMA | | |
| 9283 | BCMA, | L363(+1.5), U266(+1.5), LNCaP(+1) | ND |
| | PSMA | | |
| 9284 | BCMA, | RAJI(+/−) | RS411(+1.1) |
| | PSMA | | |
| 9285 | CD19 | RAJI(+1) | ND |
| 9286 | CD19 | RAJI(+1) | ND |
| 9287 | CD19 | RAJI(+/−) | ND |
| 9288 | CD19 | RAJI(+3) | ND |
| 9289 | CD19 | RAJI(+/−) | ND |
| 9290 | CD19 | RAJI(+1) | ND |
| 9291 | CD19 | ND | RS411(+1.1) |
| 9292 | CD19 | ND | RS411(+1.1) |
| 9293 | CD19 | RAJI(+1.5) | ND |
| 9294 | CD19 | RAJI(+/−) | ND |
| 9295 | CD19 | RAJI(+/−) | ND |
| 9296 | CD19 | RAJI(+1) | ND |
| 9297 | CD19 | RAJI(+1) | ND |
| 9298 | CD19 | RAJI(+/−) | ND |
| 9299 | CD19 | RAJI(+1) | ND |
| 9300 | CD19 | RAJI(+1) | ND |
| 9301 | CD19 | RAJI(+/−) | ND |
| 9302 | CD19 | ND | RS411(+1.8) |
| 9303 | CD19 | ND | RS411(+1.5) |
| 9304 | CD19 | RAJI(+1) | ND |
| 9305 | CD19 | RAJI(+1) | RS411(+11.2) |
| 9306 | CD20 | RAJI(+/−) | RS411(−), |
| | | | RAJI(+1.1) |
| 9307 | CD20 | RAJI(+/−) | RS411(−), |
| | | | RAJI(+1.1) |
| 9314 | CD19 | RAJI(−) | |
| 9315 | CD19 | RAJI(−) | RS411(+1.4) |
| 9316 | CD19 | RAJI(+1.5) | |
| 9317 | CD19 | RAJI(−) | |
| 9318 | CD19 | RAJI(−) | RS411(+1.1) |
| 9319 | CD19 | RAJI(−) | |
| 9321 | CD19 | RAJI(−) | |
| 9323 | CD19 | RAJI(+/−) | ND |
| 9324 | CD19 | L363(−), U266(−) | |
| 9325 | CD19 | RAJI(+/−) | ND |
| 9329 | CD19 | RAJI(−) | ND |
| 9330 | CD19 | RAJI(−) | ND |
| 9331 | CD19 | RAJI(+/−) | ND |
| 9332 | CD19 | RAJI(+2) | ND |
| 9333 | CD19 | RAJI(−) | ND |
| 9334 | CD19 | RAJI(−) | ND |
| 9335 | CD19 | RAJI(+2.5) | ND |
| 9339 | CD19 | RAJI(+1.5) | |

### NK92 Cells Expressing the CD19-TARGETED SARs Demonstrate Cytotoxicity Towards CD19-Expressing NALM6 and RAJI Cells

NK92 cells were infected with lentiviral vectors encoding the indicated CD19-TARGETED SAR constructs. The cells were selected in puromycin. Parental NK92 cells or SAR-expressing NK92 cells were co-cultured with the indicated target cell lines stably expressing Gluc at an Effector: Target (E:T) ratio of 0.25:1 for 4 hours in duplicate. At the end of the co-culture period, Gluc activity was measured by Matador assay following addition of CTZ assay buffer. The results show specific increase in Gluc activity in cultures containing Nalm6-Gluc and RAJI-Gluc cells upon co-culture with NK92 cells expressing the different CD19-TARGETED SARs. In contrast, there is no significant increase in GLuc activity in cultures containing RAJI-CD19-KO, U927 and THP-1 cells, which lack CD 19 expression. This demonstrates the specificity of the assay. The NK92 cells expressing the conventional 2^{nd} generation CAR (SEQ ID NO: 5441) showed the highest cytotoxicity towards Nalm6 and RAJI cells but also show non-specific cytotoxicity towards RAJI-CD19-KO, U937 and THP-1 cells.

TABLE 58
MATADOR ASSAY

| GLUC VALUE | | | | | | STD-DEV | | | | |
|---|---|---|---|---|---|---|---|---|---|---|
| SEQ ID | | | Raji- | | | | | Raji- | | |
| NO of | | | CD19 | | | | | CD19 | | |
| SAR | Nalm6 | Raji | KO | U937 | THP1 | Nalm6 | Raji | KO | U937 | THP1 |
| Parental | 1527 | 13326 | 6018 | 2409 | 2110 | 618 | 321 | 373 | 127 | 48 |
| NK92 | | | | | | | | | | |
| SEQ ID | 7002 | 23248 | 6424 | 2761 | 1973 | 194 | 246 | 1299 | 257 | 18 |
| NO: 5134 | | | | | | | | | | |
| SEQ ID | 9550 | 29486 | 9444 | 4957 | 2148 | 69 | 160 | 245 | 64 | 185 |
| NO: 5137 | | | | | | | | | | |
| SEQ ID | 5984 | 22941 | 7527 | 3470 | 2167 | 603 | 156 | 92 | 122 | 60 |
| NO: 2291 | | | | | | | | | | |
| SEQ ID | 6525 | 23767 | 7369 | 2852 | 2034 | 100 | 261 | 711 | 8 | 64 |
| NO: 5138 | | | | | | | | | | |
| SEQ ID | 3237 | 14857 | 5994 | 2306 | 2073 | 30 | 389 | 314 | 64 | 105 |
| NO: 5136 | | | | | | | | | | |
| SEQ ID | 2240 | 16922 | 6943 | 3501 | 1975 | 120 | 1003 | 410 | 12 | 34 |
| NO: 2289 | | | | | | | | | | |
| SEQ ID | 20882 | 35788 | 13510 | 7612 | 5388 | 1030 | 66 | 438 | 183 | 420 |
| NO: 5441 | | | | | | | | | | |
| SEQ ID | 11722 | 23869 | 7729 | 2920 | 2304 | 289 | 234 | 514 | 64 | 88 |
| NO: 5440 | | | | | | | | | | |
| SEQ ID | 12025 | 22126 | 7659 | 2761 | 2302 | 239 | 1570 | 141 | 201 | 192 |
| NO: 5139 | | | | | | | | | | |

### Matador Assay

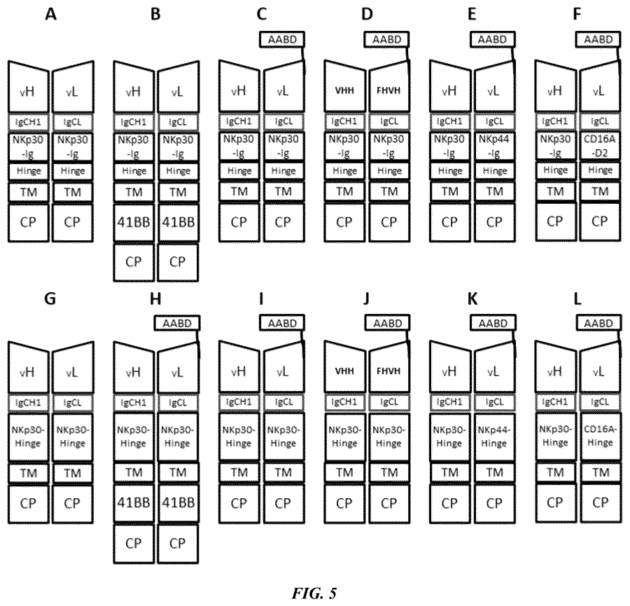

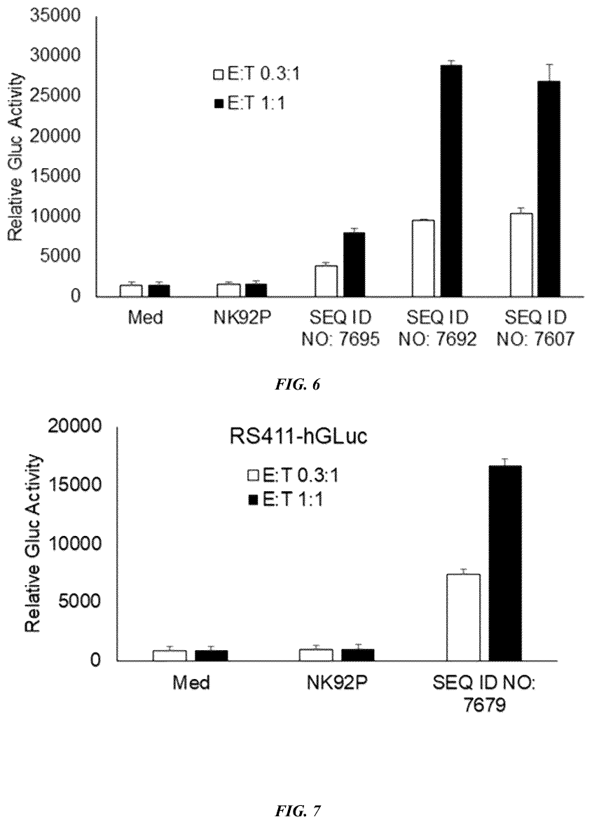

NK92 paratent and NK92 cells expressing the indicated SARs and RS4; 11-Gluc target cells were co-incubated in E:T ratios of 0.3:1 and 1:1, which is 5,000:15,000 cells and 15,000:15,000 cells in each case. After 2-hour incubation, cell death was measured using Matador assay by addition of coeleleterazine. Results are shown in FIG. 6 and show effective induction of cell death by SAR with SEQ ID NO: 7695, 7692 and 7607.

### Matador Assay

NK92 paratent and NK92 cells expressing the indicated SARs and RS4; 11-Gluc target cells were co-incubated in E:T ratios of 0.3:1 and 1:1, which is 5,000:15,000 cells and 15,000:15,000 cells in each case. After 2-hour incubation, cell death was measured using Matador assay by addition of coeleleterazine. Results are shown in FIG. 7 and show effective induction of cell death by SAR with SEQ ID NO: 7679.

### Matador Assay

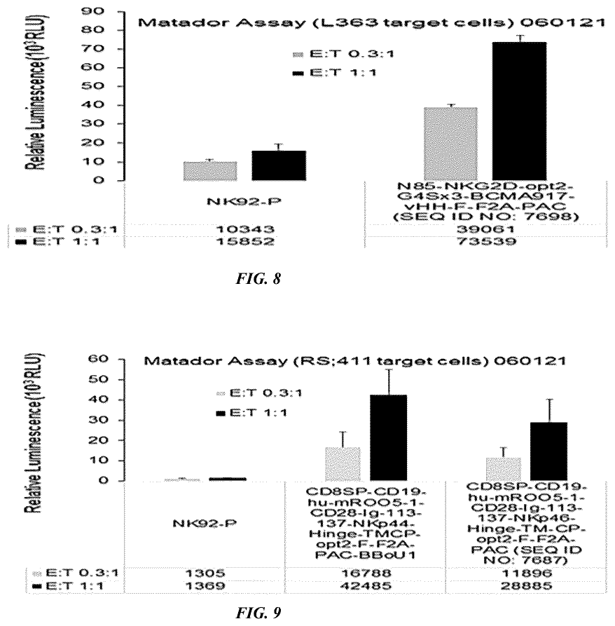

NK92 paratent and NK92 cells expressing the indicated SARs and L363-Gluc target cells were co-incubated in E:T ratios of 0.3:1 and 1:1, which is 5,000:15,000 cells and 15,000:15,000 cells in each case. After 2-hour incubation, cell death was measured using Matador assay by addition of coeleleterazine. Results are shown in FIG. 8 and show effective induction of cell death by SAR with SEQ ID NO: 7679.

### Matador Assay

NK92 paratent and NK92 cells expressing the indicated SARs and RS4; 11-Gluc target cells were co-incubated in E:T ratios of 0.3:1 and 1:1, which is 5,000:15,000 cells and 15,000:15,000 cells in each case. After 2-hour incubation, cell death was measured using Matador assay by addition of coeleleterazine. As shown both SAR constructs, comprising hu-mROO5-1-scFv, CD28 hinge region and NKp44-Hinge-TMCP or NKp46-Hinge-TMCP induced effective cell death. The results are shown in FIG. 9.

### Matador Assay

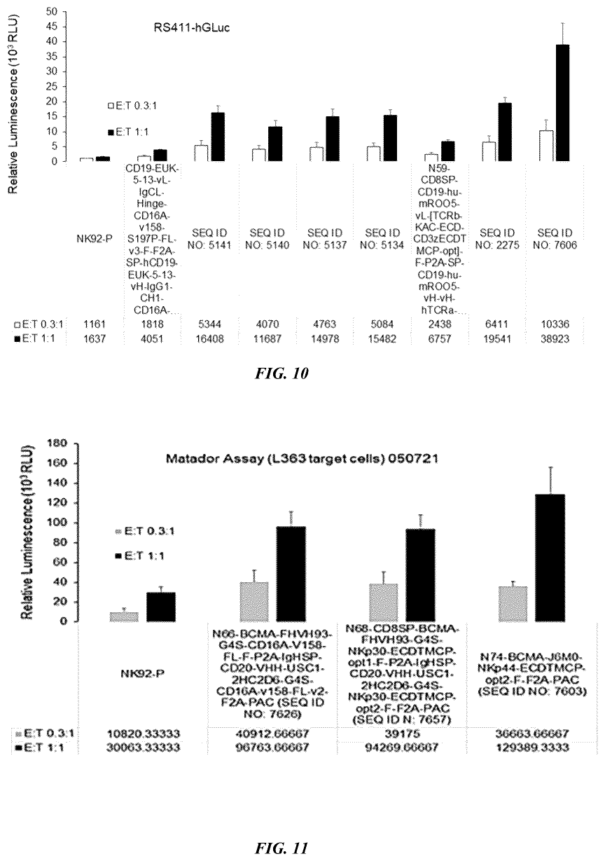

NK92 paratent and NK92 cells expressing the indicated SARs and RS4; 11-Gluc target cells were co-incubated in E:T ratios of 0.3:1 and 1:1, which is 5,000:15,000 cells and 15,000:15,000 cells in each case. After 2-hour incubation, cell death was measured using Matador assay by addition of coeleleterazine. Results are shown in FIG. 10

### Matador Assay

NK92 paratent and NK92 cells expressing the indicated SARs and L363-Gluc target cells were co-incubated in E:T ratios of 0.3:1 and 1:1, which is 5,000:15,000 cells and 15,000: 15,000 cells in each case. The NK92 and NK92-SAR cells also co-expressed a membrane anchored form of IL2 (SEQ ID NO: 7133). After 2-hour incubation, cell death was measured using Matador assay by addition of coeleleterazine. Results are shown in FIG. 11.

### Matador Assay

NK92 cells were engineered to express the SAR constructs NKG2D-opt2-G4Sx3-Bst-Her2-47D5-vHH-Mlu-F-F2A-Xba-PAC (SEQ ID NO: 7696) and NKG2D-opt2-G4Sx3-Bst-Her3-21F06-vHH-Mlu-F-F2A-Xba-PAC (SEQ ID NO: 7697). The NK92 paratent and NK92 cells expressing the SARs are co-cultured with SKOV3-Gluc target cells at an E:T ratios of 1:1. Cell death was measured using Matador assay by addition of coeleleterazine. Results show increase in Gluc activity upon co-culture with NK92 cells expressing the SAR constructs with SEQ ID NO: 7696 and 7697, reflecting induction of cell death.

### SAR-NK Cells

NY-ESO1-Tetramer-APC (HLA-A*02:01 human NY-ESO1 157-165 C165V APC-Labeled Tetramer) that target NY-ESO1 (157-165) peptide (SEQ ID NO: 10880) were obtained from NIH tetramer facility. JNG cells infected with NY-ESO1-SIR (061621-SCjJ7; SEQ ID NO: 9366) were selected with puromycin. The stable cells were stained with NY-ESO1-Tetramer-APC (HLA-A*02:01 human NY-ESO1 157-165 C165V APC-Labeled Tetramer) for 30 min at room temperature in dark and analyzed by flow cytometry. JNG cells expressing NY-ESO1 SAR were APC positive (10%) as compared to JNG-UI cells (1.42%). Similarly, 293FT cells transiently transfected with NY-ESO1-SAR (061621-SCjJ7; SEQ ID NO: 9366) and stained with NY-ESO1-tetramer-APC were 52.3% APC positive as compared to 293FT un-transfected cells (0.97%). The experiment was repeated with a construct in which the Va domain of the SAR was replaced by a different Va domain (SEQ ID NO: 8514). 293FT cells transfected with this SAR construct showed >70% staining with the NY-ESO1-tetramer-APC demonstrating that the platform can be used to construct different SAR with TCR like binding ability and comprising different variable domains. Next, the linker domains (i.e., TCRa-Ig3 and TCRb-Ig3) and signaling modules (e.g., CD3zECDTMCP-opt and CD3zECDTMCP opt2) of the NY-ESO1-SIR (061621-SCjJ7; SEQ ID NO: 9366) constructs were replaced by different linkers (e.g., IgCL, IgG-CHI etc.) and signaling modules comprising different signaling adaptors and variants and fragments thereof. The resulting SAR constructs are represented by SEQ ID NO: 9427-9434. The constructs showed increased staining with the NY-ESO1-tetramer-APC when transfected in 293FT cells. Other exemplary constructs targeting NY-ESO1 and comprising different backbones are represented by SEQ ID NO: 9356-9426 and are tested similarly. uTCR-SAR constructs targeting MAGE-A3 (112-120)/HLA-A2, (SEQ ID NO: 9439-9506, 9517-9518) are tested similarly using a MAGE-A3 (112-120)-tetramer-APC obtained from NIH tetramer facility.

THP-1 cells are stably transduced with the lentiviral vector encoding uTCR-SAR NY-ESO1-SAR (061621-SCjJ7; SEQ ID NO: 9366). The cells show increase in staining with the NY-ESO1-tetramer-APC and increase in phagocytosis of target cells with surface NY-ESO1/HLA-A2 complex expression.

A concentrated lentiviral vector encoding NY-ESO1-SAR (061621-SCjJ7; SEQ ID NO: 9366) was used to infect NK92 cell line, primary T cells and primary NK cells. Following infection, cells were stained with the NY-ESO1-tetramer-APC as above. Expression of the NY-ESO1-SAR (061621-SCjJ7; SEQ ID NO: 9366) resulted in increase in APC+ve cells in NK92 cell line from 4.93% to 84.06%, in primary NK cells from 3.3% to 10.98% and in primary T cells from 29% to 59.3%. These results demonstrate that NY-ESO1-SAR can be functionally expressed in variety of cell line, including both T cells and non-T cells and can confer TCR-like binding ability on those cells.

NK92, primary NK, and primary T cells expressing NY-ESO1-SAR (061621-SCjJ7; SEQ ID NO: 9366) were grown for 1 day in XVIVO medium supplemented with 50 IU/ml IL2. Co-cultured with T2-cells expressing GLuc in the presence and absence of a NYESO peptide (10μM), a CD28 agonist antibody (1 μg/ml), NY-ESO-1 peptide+CD28 antibody was carried out. T2 cells were load with the NY-ESO1 peptide for 30 min at 37C. In addition, T2 cells infected with a lentiviral vector (020122-BBjV1) expressing an exogenous HLA-A2 coding sequence along with an NY-ESO-1 coding sequence were included as controls. In addition, Gluc expressing L363 (NY-ESO1+/HLA-A2+) and U266 (NY-ESO1+/HLA-A2+) cells. L363 and U266 cells transduced with the 020122-BBjV1 vector were included. All target and effector cells were plated at an E:T ratio 1:1 in a white 384-well plate for 4h in XVIVO media without any supplements. Target cells were used at 10 thousand cells/well in 30μl of medium. Matador assay was performed after injection of 15μl of 1:100 CTZ assay buffer in PBS in a well-mode using an automated dispenser. Luminescence was read for 5 seconds. The results showed a marked increase in Gluc activity reflecting cell death in cultures of U266-Gluc cells with NK92, primary NK and primary T cells that had been infected with the NY-ESO1-SAR (061621-SCjJ7; SEQ ID NO: 9366) as compared to the uninfected control cells. The Gluc activity increased from 23356 in cultures of NK92 to 334646 in NK92 expressing NY-ESO1-SAR. The Gluc activity increased from 17788 in cultures of primary NK cells to 162764 in primary NK expressing NY-ESO1-SAR. The Gluc activity increased from 2183 in cultures of primary T cells to 491493 in primary T cells expressing NY-ESO1-SAR. Similarly, expression of NY-ESO1-SAR (061621-SCjJ7; SEQ ID NO: 9366) in NK92, primary NK and primary T cells showed increased cytotoxicity towards L363 cells and L363 cells stably transduced with the 020122-BBjV1 vector as compared to the uninfected control cells. Finally, expression of NY-ESO1-SAR (061621-SCjJ7; SEQ ID NO: 9366) in NK92 and primary NK cells showed increased cytotoxicity towards T2 cells that had been load with the NY-ESO1 peptide as compared to T2 cells that had been loaded with the peptide.

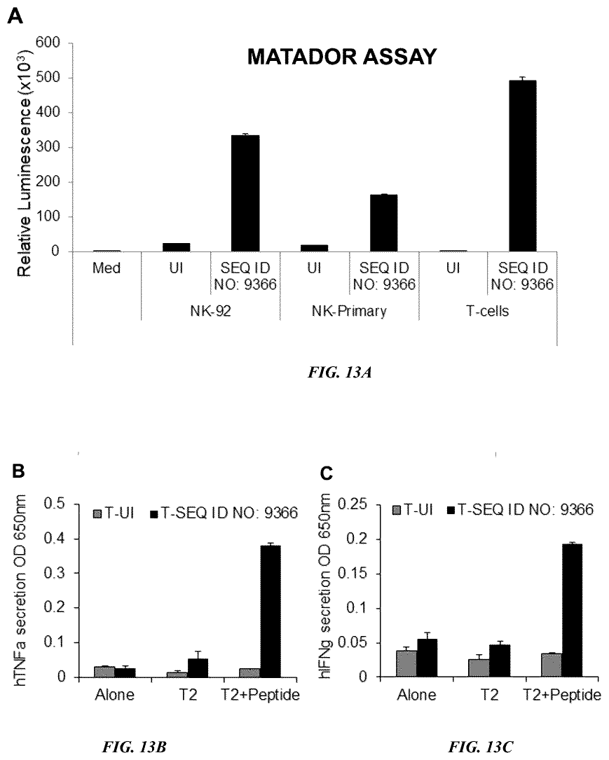

Control T2 cells or T2 cells that had been loaded with the NY-ESO1 peptide were plated at 50K cells/well in100μl culture medium in a 96 well U bottom plate. Control effector cells (NK92 and primary T cells) or effector cells that had been infected with the NY-ESO1-SAR encoding lentiviral vector (061621-SCjJ7; SEQ ID NO: 9366) were added at an E:T ratio of 1:1. After 24h, supernatant was collected for ELISA. The results showed increase in IFNγ and TNFα production in T2 cells that been loaded with the NY-ESO1 peptide when co-cultured with the NK92 cells and primary T cells expressing the NY-ESO1-SAR (061621-SCjJ7; SEQ ID NO: 9366) as compared to uninfected control cells (FIG. 13). This effect was specific to T2 cells that been loaded with the NY-ESO1 peptide and was not seen to the same magnitude in T2 cells that had not been loaded with the NY-ESO1 peptide.

NK92, primary NK and primary T cells are infected with a lentiviral construct expressing a uTCR-SAR CD8SP-MAGE-A3-112-120-Vb-TCRb-S57C-ECD-CD3zECDTMCP-opt-F-P2A-MAGE-A3-112-120-Va-TCRa-T48C-ECD-CD3zECDTMCP-opt2 (SEQ ID NO: 9450) targeting a MAGE-A3 peptide (112-120) in complex with HLA-A2. The experiment is repeated as above using T2 cells that had been loaded with the MAGE-A3 peptide (SEQ ID NO: 10879). Co-culture with uTCR-SAR expressing cells is shown to result in increase in cytotoxicity and cytokine (IFNγ and TNFα) production as compared with co-culture with untransduced cells. T2 cells that are not loaded with the peptide serve as negative control.

Essentially a similar approach as described above can be used to generate primary macrophage expressing uTCR-SAR targeting NY-ESO1, MAGE-A3 and other intracellular peptide antigens.

Essentially a similar approach as described above can be used to generate and test uTCR-SAR targeting other peptide antigens. The SEQ ID of several additional exemplary unispecific and bispecific uTCR-SAR constructs comprising the variable domains of TCR targeting NY-ESO1, MAGE-A3, MC7.G5 (an HLA-independent TCR) and Vd2/Vg9 (a yδ TCR) are presented in SEQ ID NO: 9355-9602. These constructs can be expressed in primary NK cells, primary T cells, NK cell lines, iPSC cells, hematopoietic cells and other effector cells (e.g., CIK, memory like NK, g-NK dendritic cells etc.) and tested for activity using techniques known in the art.

### Expression of CD19-TARGETED SARs on THP-1 Cells

THP-1 (Monocyte) cells obtained from ATCC were infected with lentiviral vectors encoding the indicated SAR constructs targeting CD19. The cells were selected in puromycin. The ability of CD19-Targeted SAR-expressing THP-1 cells to bind to CD19 extracellular domain was tested by Topanga assay using FLAG-CD19-ECD-GGSG-NLuc-AcV5 (SEQ ID NO: 3675) as described previously (Gopalakrishnan, R et al, Sci. Reports, 9:1957, 2019). The results show increased binding of the CD19 Topanga reagent to THP-1 cells expressing the SARs represented by SEQ ID NO: 2312, 2291, 5138, 2313 as compared to the parental THP-1 cells. These results demonstrate that these SARs can be functionally expressed on the surface of THP-1 cells of monocyte lineage and show increased binding to CD19 target antigen.

TABLE 59
TOPANGA ASSAY

| SEQ ID NO | CELL LINE | Average | STD DEV |
|---|---|---|---|
| | THP1-Parental | 119 | 7 |
| SEQ ID | CD8SP-hCD19-EUK-5-13-vL-IgCL-NKp46-Hinge- | 969 | 240 |
| NO: 2312 | TM-CP-opt2-F-F2A-SP-hCD19-EUK-5-13-vH-IgG1- | | |
| | CH1-NKp46-Hinge-TM-CP-opt1-F-P2A-PAC | | |
| SEQ ID | CD8SP-hCD19-EUK-5-13-vL-IgCL-Xho- | 1651 | 183 |
| NO: 2291 | CD3zECDTMCP-opt-F-P2A-Spe-SP-Bst-hCD19- | | |
| | EUK-5-13-vH-IgG1-CH1-Mlu-CD3zECDTMCP- | | |
| | opt2-F-F2A-PAC | | |
| SEQ ID | CD8SP-FMC63-vL-V5-[hTCRbECD-Bam- | 719 | 98 |
| NO: 5138 | CD3zECDTM-28z-opt]-F-P2A-SP-FMC63-vH-Myc- | | |
| | [hTCRaECD-Kpn-CD3zECDTM-28z-opt2]-F-F2A- | | |
| | PAC | | |
| SEQ ID | CD8SP-hCD19-EUK-5-13-vL-IgCL-Bam-DAP10- | 706 | 9 |
| NO: 2313 | opt1-Spe-CD3zCP-opt1-F-P2A-dSPE-IgSP-Bst- | | |
| | hCD19-EUK-5-13-vH-IgG1-CH1-Kpn-DAP10-opt2- | | |
| | Xba-CD3zCP-opt2-F-F2A-dXba-Nde-PAC | | |

### THP-1 Cells Expressing CD19-TARGETED SAR Demonstrate Increased Phagocytosis of CD19+ RAJI Cells

7.5×10⁴ THP-1 cells expressing the indicated SAR constructs against CD19 and THP-1 parental cells were differentiated to monocyte/macrophage lineage using 1 ng/mL of PMA (phorbol 12-myristate 13-acetate) in RPMI with 10% FBS for 48 hours in triplicate in two sets. The cells became adherent and were washed twice. 7.5×110⁴ RAJI-Nluc and RAJI-CD19-KO-Nluc cells target cells were then added to the appropriate wells for 3-4 hours. Suspension cells were removed and the plate was washed twice. 500 μL of EDTA in PBS was added and incubated for 5 minutes at 37° C. The cells were scrapped off, put them into tubes and spun at 1000 rpm for 5 minutes at 4° C. The PBS was removed and 100 μL 1×Renilla Luciferase Assay Lysis Buffer (Promega) was added to the tubes and incubated on ice for 10 minutes. Samples were spun at 12,000 for 10 minutes at 4° C. 25μL of supernatant was collected in triplicate with 25μL of CTZ (Coelentrazine) assay buffer was added and luminescence was measured using a plate reader. As shown in the following Table 60, THP-1 cells expressing SARs with SEQ ID NO: 2291 and 5138 showed higher NLuc activity as compared to THP-1 parental cells upon co-culture with RAJI cells. These results demonstrate increased phagocytosis of CD19-expressing RAJI cells by the CD19-TARGETED-SAR expressing THP-1 cells. In contrast, THP-1 cells expressing SARs with SEQ ID NO: 2291 and 5138 showed no increase in NLuc activity as compared to THP-1 parental cells upon co-culture with RAJI-CD19-KO cells that lack CD19 expression.

TABLE 60

| | Average | | | STD DEV | | |
|---|---|---|---|---|---|---|
| | | | +RAJI- | | | +RAJI- |
| SEQ ID NO | Alone | +RAJI | CD19-KO | Alone | +RAJI | CD19-KO |
| THP1-Parental | 32 | 40943 | 5053 | 7 | 34225 | 791 |
| SEQ ID NO: | 173 | 93208 | 3106 | 153 | 32938 | 1559 |
| 2291 | | | | | | |
| SEQ ID NO: | 272 | 163293 | 3013 | 29 | 35872 | 1628 |
| 5138 | | | | | | |
| RAJI | 13509 | | | 232 | | |
| RAJI-CD19-KO | 412 | | | 15.3 | | |

### Expression of Multipurpose Switches.

NK92 cells were stably transduced with lentiviral vectors encoding a SAR and coexpressing accessory module comprising different membrane anchored cytokines or multipurpose switches. Exemplary constructs expressing a Synth-IL2-tBCMA-L24 multipurpose switch are represented by SEQ ID NO: 8509-8512. The NK92 cells were withdrawn from IL2 following infection. The co-expression of accessory modules represented by SEQ ID NO: 7133-7137, 7151-7157 and 8529-8534 resulted in survival of NK92 cells when grown in medium lacking IL2, while the control untransduced NK92 cells died. The NK92 cells expressing the different SAR constructs showed robust expression and activity of the SAR as measured by Matador assay. The NK92 cells expressing the multiple purpose switches representing IL2-tHer2 (SEQ ID NO: 8533) IL2-RQR8 (SEQ ID NO: 8529) and IL2-tBCMA (SEQ ID NO: 7151) were stained with Herceptin, Rituximab and J6M0 antibodies that bind to Her2, RQR8 and BCAM respectively and were found to show positive staining. In addition, NK92 cells expressing IL2-RQR8 (SEQ ID NO: 8529) also show staining with QBEND-10 antibody that binds to CD34. JNG cells expressing the above multipurpose switches also show cell surface expression of the multipurpose switch when detected using the above-described antibodies. These results show that SAR expressing cells can be detected, isolated and purified by staining with the antibodies that bind to the multipurpose switch followed by cell sorting (e.g., flow sorting or magnetic sorting). These results further show that SAR expressing cells can be eliminated by staining with the antibodies that bind to the multipurpose switch followed by negative selection using cell sorting (e.g., flow sorting or magnetic sorting). Furthermore, cells expressing the multipurpose switch are killed by treatment with an antibody that binds to the switch, such as Herceptin, rituximab, J6M0 or a BCMA-ADC.

It was also observed that constructs in which the accessory modules are expressed using a short internal promoter (e.g., EFS, EFS2, RSV etc.) show superior expression of the SAR and the accessory module. This was particularly seen in the case of SAR constructs that have two chains.

Use of autologous SAR-T cells targeting multiple antigens for adoptive cell therapy. Patients many different diseases, including infectious diseases (e.g., HIV1, EBV, CMV, HTLV1, etc.), degenerative diseases (e.g., Alzheimer's disease), autoimmune disease (e.g., pemphigous vulgaris), allergic diseases (e.g., chronic idiopathic urticarial) and multiple cancers are enrolled in an IRB approved phase I clinical trial of immunotherapy with adoptively transferred autologous SAR-T cells targeting different disease causing or disease associated antigens. The SAR for different diseases are selected based on the known expression of their target antigen in the disease causing or disease associated cells. Where possible, the expression of the SAR target on the disease causing or disease associated cells is confirmed by binding with ABD-GGS-NLuc fusion protein in which the antigen binding domain of SAR fused to non-secretory form of NLuc protein via a flexible linker. Alternatively, immunohistochemistry or flow cytometry using commercially available antibodies is used to confirm the expression of the SAR target on disease causing or disease associated cells. T cells are collected from the subject using leukapheresis, transduced with the appropriate SAR encoding lentivirus vector and expanded ex vivo using CD3/CD28 beads. After the resulting cell products have undergone quality control testing (including sterility and tumor specific cytotoxicity tests), they are cryopreserved. Meanwhile, study participants commence with lymphodepletive chemotherapy (30 mg/m²/day fludarabine plus 500 mg/m²/day cyclophosphamide x 3 days). One day after completion of their lymphodepleting regimen, the study participant receives transduced lymphocytes infused intravenously followed by high-dose (720,000 IU/kg) IL-2 (Aldesleukin; Prometheus, San Diego, CA) every 8 hours to tolerance. The previously stored SAR-T cell product is transported, thawed and infused at the patient's bedside. The dose of SAR-T product varies from 1×10⁴ SAR+ve CD3 cells/kg to 5×10⁹ SAR+ve CD3 cells/kg as per the study protocol. The SAR-T product may be administered in a single infusion or split infusions. Research participants can be pre-medicated at least 30 minutes prior to T cell infusion with 15 mg/kg of acetaminophen P.O. (max. 650 mg.) and diphenhydramine 0.5-1 mg/kg I.V. (max dose 50 mg). The study participant may optionally receive daily injections of human IL-2. Clinical and laboratory correlative follow-up studies can then be performed at the physician's discretion.

Use of allogeneic SAR-T cells for adoptive cells therapy. Patients with relapsed Acute Lymphocytic Leukemia or high-risk intermediate grade B-cell lymphomas who have undergone an allogeneic bone marrow transplant may receive immunotherapy with adoptively transferred allogeneic SAR-T cells. A leukapheresis product collected from the donor (same donor as used for the allogeneic transplant) undergoes selection of CD3 positive T lymphocytes using the CliniMACS Prodigy® System from Miltenyi Biotec and following the manufacturer's recommendations. The expression of TRAC and 02M is eliminated by CRISP9 mediated knock-out using techniques known in the art and T cells lacking cell surface expression of TCR/CD3 complex and HLA are selected. Cells are activate using a CD3 and CD28 magnetic bead-based artificial antigen presenting cells and transduced with clinical grade CD20-targeted SAR virus (e.g., CD8SP-CD20-VHH-USC1-CD16A-F158V-S197P-FL-v3 (SEQ ID NO: 5047]). Cells are expanded for 9-12 days in a closed system. After the resulting cell products have undergone quality control testing (including sterility and tumor specific cytotoxicity tests), they are cryopreserved. Meanwhile, study participants commence with lymphodepletive chemotherapy (30 mg/m²/day fludarabine plus 500 mg/m²/day cyclophosphamide x 3 days). One day after completion of their lymphodepleting regimen, the study participant receives transduced lymphocytes infused intravenously followed by high-dose (720,000 IU/kg) IL-2 (Aldesleukin; Prometheus, San Diego, CA) every 8 hours to tolerance. The SAR-T cell product is transported, thawed and infused at the patient's bedside. The dose of SAR-T product may vary from 1×10⁴ SAR+ve CD3 cells/kg to 5×10⁹ SAR+ve CD3 cells/kg as per the study protocol. The SAR product may be administered in a single infusion or split infusions. Research participants can be pre-medicated at least 30 minutes prior to SAR-T cell infusion with 15 mg/kg of acetaminophen P.O. (max. 650 mg.) and diphenhydramine 0.5-1 mg/kg I.V. (max dose 50 mg). Clinical and laboratory correlative follow-up studies can then be performed at the physician's discretion, and may include quantitative RT-PCR studies for the presence of CD20-expressing ALL/lymphoma cells and/or the adoptively transferred T cells; FDG-PET and/or CT scans; bone marrow examination for disease specific pathologic evaluation; lymph node biopsy; and/or long-term follow up per the guidelines set forth by the FDA's Biologic Response Modifiers Advisory Committee that apply to gene transfer studies. Use of immunosuppressive drugs is also at the discretion of the physician. Essentially a similar approach can be used to treat other diseases using allogeneic immune cells (e.g., T cells) expressing the SAR of the disclosure where the SAR targets an antigen or antigens expressed on the disease causing or disease-associated cells. Essentially a similar protocol is used to test other SAR constructs listed in Tables 36-38.

### Use of Autologous or Allogeneic NK Cells Expressing SAR

A leukapheresis product collected from the donor (same donor as used for the allogeneic transplant) undergoes selection of NK cells using the CliniMACS Prodigy® System from Miltenyi Biotec and following the manufacturer's recommendations. NK cells are activated using hIL2 for 3-5 days and then transduced with a lentivirus vector encoding a CD20-targeted SAR (e.g., CD8SP-CD20-VHH-USC1-CD16A-F158V-S197P-FL-v3 (SEQ ID NO: 5047]). The lentivirus also encodes for a membrane anchored form of IL2 (SEQ ID NO: 1330). NK cells are expanded ex vivo for 15 days in the presence of artificial antigen presenting K562 cells (aAPCs) expressing human CD20 with 50 units/mL of hIL-2. After the resulting cell products have undergone quality control testing (including sterility and tumor specific cytotoxicity tests), they are cryopreserved. Meanwhile, study participants commence with lymphodepletive chemotherapy (30 mg/m2/day fludarabine plus 500 mg/m2/day cyclophosphamide x 3 days). One day after completion of their lymphodepleting regimen, the study participant receives transduced NK cells infused intravenously followed by high-dose (720,000 IU/kg) IL-2 every 8 hours to tolerance. The SAR-NK cell product is transported, thawed and infused at the patient's bedside. The dose of SAR-NK product may vary from 1×10⁴ SAR+ve NK cells/kg to 5×10⁹ SAR+ve NK cells/kg as per the study protocol. The SAR-NK product may be administered in a single infusion or split infusions.

Generation of iPSC-Derived NK Cells Expressing SAR

The CD19-targeted SAR construct CD8SP-CD19-FHVH-354-CD16A-F158V-S197P-FL-v3 (SEQ ID NO: 5042) will be expressed in two different cord stem cell derived iPSC cell lines (606A1, NCRM-1) and a peripheral blood derived iPSC cell line (648A1). Single cell clones expressing SAR as determined by binding with Topanga reagent and FACS using anti-FLAG-FITC will be isolated, expanded and undergo QC analysis (e.g., Chromosomal Integrity, pluripotency, identity confirmation, mycoplasma and sterility). Several independent clones derived from each iPSC cell line will be frozen in liquid nitrogen to serve as master cell banks.

The derivation of NK cells from iPSCs and SAR transfected iPSCs will be performed using protocols known in the art. Briefly, 3,000 TrypLE-adapted iPSCs will be seeded in 96-well round-bottom plates with APEL culture containing 40 ng/ml human Stem Cell Factor (SCF), 20 ng/ml human Vascular Endothelial Growth Factor (VEGF), and 20 ng/ml recombinant human Bone Morphogenetic Protein 4 (BMP-4). After day 11 of hematopoietic differentiation, cells will be evaluated for hematopoietic progenitor cells by flow cytometry for CD34+/CD43+ and CD34+/CD45+.

Spin embryoid bodies (EBs) will be then directly transferred into each well of uncoated 24-well plates under a condition of NK cell culture. Cells will then further differentiated into NK cells using 5 ng/mL IL-3 (first week only), 10 ng/mL IL-15, 20 ng/mL IL-7, 20 ng/mL SCF, and 10 ng/mL flt3 ligand for 28-32 days. Half-media changes will be performed weekly. NK cells will be harvested for expansion using irradiated mbIL-21 expressing artificial antigen presenting K562 cells (aAPCs) expressing human CD19 in the presence of 50 units/mL of hIL-2. After in vitro potency testing using Matador assay, the cells will be used for in vivo studies using NALM6 xenograft model in NSG mice. After additional sterility and potency assays, the cells will be used for human clinical trial for treatment of patients with CD19 expressing B cell acute lymphocytic leukemia (B-ALL), chronic lymphocytic leukemia and diffuse large B cell lymphoma.

Essentially a similar procedure would be used for generation of iPSC derived T cells expressing SAR of the disclosure using protocols for differentiation of iPSC into T cells known in the art.

### Generation and Use of NK92 Cells Expressing SARs

cGMP grade NK92 cells will be transduced with a lentivirus vector encoding a CD20-targeted SAR (e.g., CD8SP-CD20-VHH-USC1-CD16A-F158V-S197P-FL-v3 (SEQ ID NO: 5047]). The lentivirus also encodes for a membrane anchored form of IL2 (SEQ ID NO: 1330). NK92 cells are expanded ex vivo for 15 days in the presence of artificial antigen presenting K562 cells (aAPCs) expressing human CD20 and 50 units/mL of hIL-2. The cells will be y-irradiated. After the resulting cell products have undergone quality control testing (including sterility and tumor specific in vitro and in vivo cytotoxicity tests), the cells will be cryopreserved. Meanwhile, study participants commence with lymphodepletive chemotherapy (30 mg/m2/day fludarabine plus 500 mg/m2/day cyclophosphamide×3 days). One day after completion of their lymphodepleting regimen, the study participant receives transduced NK92 cells infused intravenously. The dose of SAR-NK product may vary from 1×10⁴ SAR+ve NK92 cells/kg to 5×10⁹ SAR+ve NK92 cells/kg as per the study protocol. The SAR-NK92 product may be administered in a single infusion or split infusions.

Generation of hematopoietic stem cells engineered to express a SAR CD34 positive hematopoietic stem cells are purified from G-CSF mobilized leukapheresis product and transduced with a lentiviral vector encoding a CD19-targeted SAR represented by CD8SP-CD19-FHVH-354-CD16A-F158V-S197P-FL-v3 (SEQ ID NO: 5042). The expression of endogenous CD19 in the hematopoietic stem cells is optionally eliminated using techniques described in U.S. Ser. No. 10/660,919. The subject receives myeloablative chemotherapy followed by infusion of gene-modified stem cells. Essentially a similar approach is used to express a CD33-targeted SAR (e.g., SEQ ID NO: 4871) in combination with elimination of endogenous CD33 in hematopoietic stem cells. Essentially a similar approach is used to express an MPL-targeted SAR (e.g., SEQ ID NO: 4919) in combination with elimination of endogenous MPL in hematopoietic stem cells.

### Generation and Use of SAR Expressing Macrophage/Monocytes

A CD19-targeted SAR represented by CD8SP-CD19-FHVH-354-CD16A-F158V-S197P-FL-v3 (SEQ ID NO: 5042) is expressed in macrophage/monocytes and used for the treatment of CD19-expressing lymphoma using methods described in WO2019152781.

SAR-T/NK Cell Hepatic Arterial Infusion. In addition to intravenous infusion, SAR-T and SAR-NK cells can be infused intra-arterially to provide high concentration of SAR-expressing cells in a local area or organ involved with a disease.

Intraperitoneal administration of SAR-T/NK cells. SAR-T/NK cells can also be administered intraperitoneally, essentially as described in Koneru M et al (Journal of Translational Medicine; 2015; 13:102).

Use of SAR-T/NK cells for intratumoral injection. SAR-T/NK cells can also be administered intra-tumorally, essentially as described in Brown C E, et al, Clin Cancer Res. 2015 September 15; 21(18): 4062-4072.

### Combination of Different SAR-Expressing Cells

The patient may receive a combination of different SAR expressing cells that target one or more than one antigen. For example, a patient may receive CD19-targeted SAR-T and CD20-targeted SAR-NK and CD22-targeted SAR-macrophages. Alternatively, a subject may receive CD19-targeted SAR-T, CD19-targeted SAR-NK cells and CD19-targeted SAR-macrophages.

The combination of SARs may be used to fine tune the immune response so that signaling is triggered only upon a threshold effect is achieved. Thus, an NK cell expressing a SAR targeting BCMA with SEQ ID NO: 7601 and a SAR targeting CD19 with SEQ ID NO: 7607 may show an additive or a synergistic effect when exposed to cells expressing both BCMA and CD19. The additive/synergistic effects can be achieved by targeting of different antigens (e.g., BCMA and CD19). Another approach to achieve additive/synergistic effect is through targeting of the same antigen but via different SAR receptors that comprise different signaling chains. For example, NK cells expressing a CD19-SAR with SEQ ID NO: 7607 (CD8SP-CD19-hu-mROO5-1-scFv-NKp30-ECDTMCP-opt2-F-F2A-PAC) can also co-express another CD19-SAR with SEQ ID NO: 7676 (CD8SP-CD19-hu-mROO5-1-CD16A-v158-S197P-FL-v3-F-F2A-PAC). In an alternate embodiment, the two SAR may target different epitopes of the same antigen. For example, NK cells expressing a CD19-SAR with SEQ ID NO: 7607 (CD8SP-CD19-hu-mROO5-1-scFv-NKp30-ECDTMCP-opt2-F-F2A-PAC) can also co-express another CD19-SAR with SEQ ID NO: 7660 (FMC64-CD16A-v158-S197P-FL-v3-F-F2A-PAC) or a CD19-SAR with SEQ ID NO: 7668 (hCD19-Bul2-CD16A-v158-S197P-FL-v3-F-F2A-PAC).

In another embodiment, a SAR comprising CD16 signaling chains can be combined with a SAR comprising NKp30, NKp44, NKp44, NKG2D, Dap10 and/or CD3z signaling chains to have additive/synergistic effects. In another embodiment, a SAR comprising NKp30 signaling chains can be combined with a SAR comprising CD16, NKp44, NKp46, NKG2D, Dap10 and/or CD3z signaling chains to have additive/synergistic effects. Similarly, a SAR comprising NKp44 signaling chains can be combined with a SAR comprising CD16, NKp30, NKp46, NKG2D, Dap10 and/or CD3z signaling chains to have additive/synergistic effects. Similarly, a SAR comprising NKp46 signaling chains can be combined with a SAR comprising CD16, NKp30, NKp44, NKG2D, Dap10 and/or CD3z signaling chains to have additive/synergistic effects. Similarly, a SAR comprising DAP10 signaling chains can be combined with a SAR comprising CD16, NKp30, NKp44, NKp46, NKG2D, and/or CD3z signaling chains to have additive/synergistic effects. Similarly, a SAR comprising CD3z signaling chains can be combined with a SAR comprising CD16, NKp30, NKp44, NKp46, NKG2D, and/or DAP10 signaling chains to have additive/synergistic effects. Similarly, a SAR comprising NKG2D signaling chains can be combined with a SAR comprising CD16, NKp30, NKp44, NKp46, CD3z, and/or DAP10 signaling chains to have additive/synergistic effects.

1. An at least one recombinant polynucleotide encoding a synthetic a antigen receptor (SAR) that specifically binds to a target antigen, wherein the SAR comprises a dimer of two polypeptide chains comprising:
(i) a first module comprising one or more heterologous antigen binding domains selected from the group consisting of:
a) an antibody;
b) an antibody fragment;
c) a heavy chain variable region of an antibody (vH domain) or a fragment thereof;
d) a light chain variable region of an antibody (vL domain) or a fragment thereof;
e) a single chain variable fragment (scFv) or a fragment thereof;
f) a single domain antibody (SDAB) or a fragment thereof;
g) a vHH domain or a fragment thereof;
h) a monomeric variable region of an antibody;
i) a single vH domain (SVH) or a fragment thereof;
j) a single vL domain (SVL) or a fragment thereof;
k) a non-immunoglobulin antigen binding scaffold optionally selected from the group consisting of a DARPIN, an affibody, an affilis, an adnectin, an affitin, an obody, a repebody, an fynomer, an alphabody, an avimer, an atrimer, a centyrin, a pronecti, an anticalins, a kunitz domain, an Armadillo repeat protein, a D domain, and a fragment of any of the foregoing;
l) a ligand-binding domain of a receptor or a fragment thereof;
m) a receptor-binding domain of a ligand;
n) a bispecific-antibody;
o) an autoantigen or a fragment thereof;
p) an adaptor binding domain or a fragment thereof;
q) an Fc binding domain or a fragment thereof;
r) a TCR or an HLA-independent TCR or a fragment thereof; and
s) Va, Vb, Vg or Vd fragment of a TCR or a fragment thereof,
wherein, when present, a vH domain of an antibody is attached to one polypeptide chain and the complementary vL domain of the antibody is attached to the other polypeptide chain and where the vH and the vL domains form a Fv- like antigen-binding module that specifically binds to the target antigen; or
wherein, when present, a Va domain of a TCR is attached to one polypeptide chain and the complementary Vb domain of a TCR is attached to the other polypeptide chain and where the Va and the Vb domains form a TCR-Fv-like antigen-binding module that specifically binds to the target antigen; or
wherein, when present, a Vg domain of a TCR is attached to one polypeptide chain and the complementary Vd domain of the TCR is attached to the other polypeptide chain and where the Vg and the Vd domains form a TCR-Fv-like antigen-binding module that specifically binds to the target antigen; and
(ii) a second module that comprise at least one membrane associated module (MAM), wherein the first and the second modules are operationally linked; and wherein the MAM of the first polypeptide chain (first MAM) and the MAM of the second polypeptide chain (second MAM) form a non-T cell receptor module (NTCRM) that is capable of activating at least one signaling pathway and/or recruiting at least one signaling adaptor; and
an optional third module comprising one or more autonomous antigen binding domains (AABD) that are operably linked via optional linkers to the N-terminus or near the N-terminus of the first module of one or both polypeptide chains.

2-43. (canceled)

44. The recombinant polynucleotide encoding the SAR of claim 1, wherein
a) the first polypeptide chain comprises a first antigen-binding domain comprising a vL, a Vα or a Vγ domain and a first Membrane associated module (MAM); and
b) the second polypeptide chain comprises a second antigen-binding domain comprising a vH, a Vβ or a Vδ domain and a second Membrane associated module (MAM);
wherein, when present
(i) the vL domain of the first antigen-binding domain and the complementary vH domain of the second antigen-binding domain form a Fv- like antigen-binding module that specifically binds to the target antigen; or
(ii) Vα domain of the first antigen-binding domain and the complementary VD domain of the second antigen-binding domain form a TCR-Fv-like antigen-binding module that specifically binds to the target antigen; or
(iii) Vγ domain of the first antigen-binding domain and the complementary Vδ domain of the second antigen-binding domain form a TCR-Fv-like antigen-binding module that specifically binds to the target antigen; and
wherein the first MAM and the second MAM form a non-T cell receptor module (NTCRM) that is capable of activating at least one signaling pathway and/or recruiting at least one signaling adaptor.

45. The recombinant polynucleotide encoding the SAR of claim 44, where the first polypeptide chain further comprises a first peptide linker between the first antigen-binding domain and the first MAM, and the second polypeptide chain further comprises a second peptide linker between the second antigen-binding domain and the second MAM, wherein optionally the first and/or second peptide linkers comprise, individually, one or more of the following:
(a) a constant domain of an immunoglobulin or a fragment thereof, wherein optionally the constant domain of an immunoglobulin is selected from the group consisting of CH1, CH2, CH3, CH4 or CL antibody domain, and/or comprises a sequence as set forth in any one of SEQ ID NO: 3536-3551 or a sequence with at least 70% identity thereto;
(b) a constant domain of a T cell receptor subunit or fragment thereof, wherein optionally the constant domain of a T cell receptor subunit is selected from the group consisting of Cα, Cβ, Cγ, or Cδ TCR domain, and/or comprises a sequence as set forth in any one of SEQ ID NO:3552-3569 and 9627-9631 or a sequence with at least 70% identity thereto;
and wherein optionally (a) and/or (b) comprises mutations that increase the expression, affinity and/or pairing of the two polypeptide chains and or comprise disulfide bonds that link the two polypeptide chains.

46-51. (canceled)

52. The recombinant polynucleotide encoding the SAR of claim 44,
wherein the first polypeptide further comprises a first hinge domain or fragment thereof that is located N-terminal to the first MAM; and/or wherein the second polypeptide further comprises a second hinge domain or fragment thereof that is located N-terminal to the second MAM, and wherein optionally the SAR comprises a disulfide bond between a residue in the first MAM and the second MAM and/or a residue in the first hinge domain and a residue in the second hinge domain.

53. (canceled)

54. The recombinant polynucleotide encoding the SAR of claim 44, wherein the first polypeptide further comprises a first homologous antigen binding domain or fragment thereof that is located N-terminal to the first hinge domain and/or the second polypeptide further comprises a second homologous antigen binding domain or fragment thereof that is located N-terminal to the second hinge domain, wherein the two homologous antigen binding domains are derived from the same naturally occurring non-T cell receptor as the corresponding hinge domains.

55. The recombinant polynucleotide encoding the SAR of claim 44, wherein the first polypeptide further comprises a first cytosolic domain comprising an optional activation domain, wherein the first cytosolic domain is located C-terminal to the first transmembrane/membrane-anchoring domain comprising the first MAM; and/or wherein the second polypeptide further comprises a second cytosolic comprising an optional activation domain, wherein the second cytosolic domain is located C-terminal to the second transmembrane/membrane anchoring domain comprising the second MAM, and wherein optionally the first and/or the second cytosolic domain comprises the cytosolic domain or a functional fragment thereof of one or more molecules selected from the group consisting of a z chain of the T-cell receptor complex or a homolog; a human CD3 chain wherein the human CD3 chain is optionally selected from the group consisting of CD3ƒ, CD3γ, CD3δ and CD3ε; a syk family tyrosine kinase wherein the syk family kinase is optionally selected from the group consisting of Syk and ZAP 70: a src family tyrosine kinase wherein the syk family kinase is optionally selected from the group consisting of Lck, Fyn and Lyn: a molecule involved in T-cell transduction, wherein the molecule is optionally selected from the group consisting of CD2, CD5 and CD28: a common FcR gamma (FCER1G or FcRγ or FCRG): Fc gamma RIIIa; FcR beta (Fc Epsilon Rib): CD79a, CD79b, DAP10 and DAP12.

56. The recombinant polynucleotide encoding the SAR of claim 44, wherein the first polypeptide chain further comprises a first accessory intracellular domain comprising one or more co-stimulatory domains or functional fragments thereof, wherein optionally the first accessory intracellular domain is located C-terminal to the first transmembrane/membrane anchoring domain of the first MAM; and/or wherein the second polypeptide chain further comprises a second accessory intracellular domain comprising one or more co-stimulatory or functional fragments thereof, wherein optionally the second accessory intracellular domain is located C-terminal to the second transmembrane/membrane anchoring domain comprising the second MAM, and wherein optionally the co-stimulatory domain comprises the co-stimulatory domain of a co-stimulatory molecule or a functional fragment thereof, wherein optionally the costimulatory molecule is selected from the group consisting of CD28, CD137 (4-1BB), CD134 (OX40), 2B4, CD27, CD81, CD2, CD5, CD8, BAFF-R, CD30, CD40, HVEM, CD278 (ICOS), ICAM, TNFR-I, TNFR-II, Fas, Toll ligand receptor, LFA-1, MHC class I molecule, BTLA, NKp30, NKp44, NKp46, GITR, CD81, CD160, DAP10, B7-H3, TNFR superfamily, Lck or a variant or a fragment thereof.

57. (canceled)

58. The recombinant polynucleotide encoding the SAR of claim 44, wherein the first and/or the second MAM and/or the first and/or the second NTCRM are comprised of the transmembrane/membrane anchored domain, optional cytosolic domain, optional hinge domain and/or optional extracellular domain of a non-T cell receptor and/or a signaling adaptor, and optionally wherein
(a) the transmembrane/membrane anchored domain, optional cytosolic domain, optional hinge domain and/or optional extracellular domain are derived from a single non-T cell receptor and/or a signaling adaptor or variants thereof, or
(b) the transmembrane/membrane anchored domain, optional cytosolic domain, optional hinge domain and/or optional extracellular domain are derived from different non-T cell receptor and/or a signaling adaptor or variants thereof; or
(c) one MAM comprises of the transmembrane domain of a non-T cell receptor or a variant thereof and the other MAM comprises the transmembrane domain of a signaling adaptor or a variant thereof; or
(d) one MAM comprises of the transmembrane domain of CD3ƒ or a variant thereof and the other MAM comprises of the transmembrane domain of FcRγ or a variant thereof; or
(e) one MAM comprises of the transmembrane domain of CD3ƒ or a variant thereof and the other MAM comprises of the transmembrane domain of CD16 or a variant thereof; or
(f) one MAM comprises of the transmembrane domain of FcRγ or a variant thereof and the other MAM comprises of the transmembrane domain of CD16 or a variant thereof; or
(g) one MAM comprises of the transmembrane domain of CD3ƒ or a variant thereof and the other MAM comprises of the transmembrane domain of DAP10 or a variant thereof; or
(h) one MAM comprises of the transmembrane domain of FcRγ or a variant thereof and the other MAM comprises of the transmembrane domain of DAP10 or a variant thereof, or (i) one MAM comprises of the transmembrane domain of CD3ƒ or a variant thereof and the other MAM comprises of the transmembrane domain of a TCR chain or a variant thereof, or
(i) one MAM comprises of the transmembrane domain of FcRγ or a variant thereof and the other MAM comprises of the transmembrane domain of a TCR chain or a variant thereof, or
(k) one MAM comprises of the transmembrane domain of CD16 or a variant thereof and the other MAM comprises of the transmembrane domain of a TCR chain or a variant thereof, or
(l) one MAM comprises of the transmembrane domain of CD3ƒ or a variant thereof and the other MAM comprises of the transmembrane domain of CD64 or a variant thereof; or
(m)one MAM comprises of the transmembrane domain of FcRγ or a variant thereof and the other MAM comprises of the transmembrane domain of CD64 or a variant thereof; or
(n) one MAM comprises of the transmembrane domain of CD3ƒ or a variant thereof and the other MAM comprises of the transmembrane domain of NKp30, NKp40 or NKp46 or a variant thereof; or
(o) one MAM comprises of the transmembrane domain of FcRγ or a variant thereof and the other MAM comprises of the transmembrane domain of NKp30, NKp40 or NKp46 or a variant thereof; or
(p) the two transmembrane/membrane anchored domains, optional cytosolic domains, optional co-stimulatory domain, optional hinge domains and/or optional extracellular domains are identical in sequence and are all derived from a) a single protein or b) different proteins.

59-62. (canceled)

63. The recombinant polynucleotide encoding the SAR of claim 58, wherein
a. the non T cell receptor is a naturally occurring receptor and is optionally selected from the group consisting of: CD16A, CD16B, CD64, CD32, NKp30, NKp44, NKp46, KIR2DL1, KIR2DL2, KIR2DL3, KIR2DL5A, KIR2DL5B, KIR3DL1, KIR3DL2, KIR3DL4, KIR2DL4, KIR2DS1, KIR2DS2, KIR2DS3, KIR2DS4, KIR2DS5, KIR3DS1, NKG2D, NKG2C, NKG2A, NKG2E, NKG2F, DNAM-1, 2B4, OX40, CD28, 4-1BB, CD27, CD81, CD2, CD5, TNFR-I, TNFR-II, Fas, CD30, CD40, CRTAM, TIGIT, CD96, SLAMF6, SLAMF7, CD100, CD160, CEACAM, ILT2, KLRG1, LAIR1, CD161, a variant of any of the foregoing, and fragments thereof; and
b. the signaling adaptor is optionally selected from the group consisting of: CD3ƒ, FcRγ, DAP10, a variant of any of the foregoing and fragments thereof.

64. The recombinant polynucleotide encoding the SAR of claim 44, wherein
a) the first MAM and the second MAM do not comprise the transmembrane domain and optionally the cytosolic domain of a CD3 chain selected from CD3ε, CD3γ, CD3δ or CD3ƒ; and/or
b) the first MAM and the second MAM do not comprise the transmembrane domain of a TCR chain and a CD3 chain; and/or
c) the first MAM and the second MAM do not comprise the transmembrane domain of CD3ƒ; and/or
d) at least one of the MAM does not comprise the transmembrane domain of CD3C;
e) at least one of the MAM does not comprise the transmembrane domain of a CD3 chain; and/or
f) one of the MAM comprises of the transmembrane domain of a T cell receptor constant chain selected from the group consisting of constant chain of TCRα, TCRβ, TCRγ, TCRδ and preTCRα.

65-67. (canceled)

68. The recombinant polynucleotide encoding the SAR of claim 44, wherein the first and/or the second polypeptide chains further comprise one or more autonomous antigen binding domains (AABD) that are attached to the N-terminus or near the N-terminus of the first and/or the second antigen binding domains, wherein optionally the one or more AABD are selected from a group consisting of a single vH domain (SVH), a single vL domain (SVL), a vHH domain, a single domain antibody, a single variable domain of a TCR (svd-TCR), a non-immunoglobulin antigen binding scaffold, a ligand-binding domain of a receptor, a receptor-binding domain of a ligand, an autoantigen, an adaptor binding domain, an adaptor, a epitope, a tag, an Fc binding domain, a fragment thereof and/or a variant thereof; and wherein a non-immunoglobulin antigen binding scaffold is optionally selection from the group of a DARPIN, an affibody, an affilis, an adnectin, an affitin, an obody, a repebody, an fynomer, an alphabody, an avimer, an atrimer, a centyrin, a pronecti, an anticalins, a kunitz domain, an Armadillo repeat protein, a D domain; and wherein optionally the AABD is a non-scFv domain.

69-70. (canceled)

71. The recombinant polynucleotide encoding the SAR of claim 1, wherein the SAR that binds to the one or more target antigens selected from the group consisting of: CD19; CD123; CD22; CD30; CD171; CS-1 (also referred to as CD2 subset 1, CRACC, SLAMF7, CD319, and 19A24); C-type lectin-like molecule-1 (CLL-1 or CLECL1); CD33; epidermal growth factor receptor variant III (EGFRviii); ganglioside G2 (GD2); ganglioside GD3; TNF receptor family member B cell maturation (BCMA); Tn antigen ((Tn Ag); prostate-specific membrane antigen (PSMA); Receptor tyrosine kinase-like orphan receptor 1 (ROR1); FmsLike Tyrosine Kinase 3 (FLT3); Tumor-associated glycoprotein 72 (TAG72); CD38; CD44v6; a glycosylated CD43 epitope expressed on acute leukemia or lymphoma but not on hematopoietic progenitors, a glycosylated CD43 epitope expressed on non-hematopoietic cancers, Carcinoembryonic antigen (CEA); Epithelial cell adhesion molecule (EPCAM); B7H3 (CD276); KIT (CD 117); Interleukin-13 receptor subunit alpha-2 (IL-13Ra2 or CD213A2); Mesothelin; Interleukin 11 receptor alpha (IL-llRa); prostate stem cell antigen (PSCA); Protease Serine 21 (Testisin or PRSS21); vascular endothelial growth factor receptor 2 (VEGFR2); Lewis(Y) antigen; CD24; Platelet-derived growth factor receptor beta (PDGFR-beta); Stage-specific embryonic antigen-4 (SSEA-4); CD20; Folate receptor alpha; Receptor tyrosine-protein kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CAlX); Proteasome (Prosome, Macropain) Subunit, Beta Type, 9 (LMP2); glycoprotein 100 (gp10O); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); Fucosyl GM1; sialyl Lewis adhesion molecule (sLe); ganglioside GM3; transglutaminase 5 (TGS5); high molecular weight-melanoma associated antigen (HMWMAA); o-acetyl-GD2 ganglioside (OAcGD2); Folate receptor beta; tumor endothelial marker 1 (TEM1/CD248); tumor endothelial marker 7-related (TEM7R); claudin 6 (CLDN6); thyroid stimulating hormone receptor (TSHR); G protein coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); Cancer/testis antigen 1 (NY-ESO-1); Cancer/testis antigen 2 (LAGE-la); Melanoma-associated antigen 1 (MAGE-A1); MAGE-A2, MAGE-A3, MAGE-A4, PRAME, PSA, ETS translocation-variant gene 6, located on chromosome 12p (ETV6-AML); sperm protein 17 (SPA17); X Antigen Family, Member 1A (XAGEl); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; surviving; telomerase; prostate carcinoma tumor antigen-1 (PCT A-1 or Galectin 8), melanoma antigen recognized by T cells 1 (MelanA or MARTI); Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin Bl; v-myc avian myelocytomatosis viral oncogene neuroblastoma derived homolog (MYCN); Ras Homolog Family Member C (RhoC); Tyrosinase-related protein 2 (TRP-2); Cytochrome P450 lB 1 (CYP1B 1); CCCTC-Binding Factor (Zinc Finger Protein)-Like (BORIS or Brother of the Regulator of Imprinted Sites), Squamous Cell Carcinoma Antigen Recognized By T Cells 3 (SART3); Paired box protein Pax-5 (PAX5); proacrosin binding protein sp32 (OY-TESI); lymphocyte-specific protein tyrosine kinase (LCK); A kinase anchor protein 4 (AKAP-4); synovial sarcoma, X breakpoint 2 (SSX2); Receptor for Advanced Glycation End products (RAGE-1); renal ubiquitous 1 (RUl); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIRI); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); and immunoglobulin lambda-like polypeptide 1 (IGLLl), MPL, Biotin, c-MYC epitope Tag, CD34, LAMP1 TROP2, GFRalpha4, CDH17, CDH6, NYBR1, CDH19, CD200R, Slea (CA19.9; Sialyl Lewis Antigen) Fucosyl-GM1, PTK7, gpNMB, CDH1-CD324, DLL3, CD276/B7H3, IL11Ra, IL13Ra2, CD179b-IGLl1, ALK TCR gamma-delta, NKG2D, CD32 (FCGR2A), Tn ag, CSPG4-HMW-MAA, Tim1-/HVCR1, CSF2RA (GM-CSFR-alpha), TGFbetaR2, VEGFR2/KDR, Lews Ag, TCR-beta1 chain, TCR-beta2 chain, TCR-gamma chain, TCR-delta chain, FITC, Leutenizing hormone receptor (LHR), Follicle stimulating hormone receptor (FSHR), Chorionic Gonadotropin Hormone receptor (CGHR), CCR4, GD3, SLAMF6, SLAMF4, HIV1 envelope glycoprotein, HTLV1-Tax, CMV pp65, EBV-EBNA3c, influenza A hemagglutinin (HA), GAD, PDL1, Guanylyl cyclase C (GCC), auto antibody to desmoglein 3 (Dsg3), autoantibody to desmoglein 1 (Dsgl), HLA, HLA-A, HLA-A2, HLA-B, HLA-C, HLA-DP, HLA-DM, HLA-DOA, HLA-DOB, HLA-DQ, HLA-DR, HLA-G, IGE, CD99, RAS G12V, Tissue Factor 1 (TF1), AFP, GPRC5D, claudin18.2 (CLD18A2 OR CLDN18A.2), P-glycoprotein, STEAPI, LIV1, NECTIN-4, CRIPTO, GPA33, BST1/CD157, low conductance chloride channel, and SARS-CoV2 Spike protein.

72. The recombinant polynucleotide encoding the SAR of claim 1, wherein the encoded SAR polypeptide comprises one or more components selected from the group consisting of:
(i) a heavy chain variable region (vH) of an antibody comprising a sequence as set forth in any of SEQ ID Nos: 2682-2918 or sequences with at least 80% identity thereto or a sequence with at least 80% identity in the three complementarity determining regions (CDRs) to the sequences set forth in any one or more of SEQ ID NOS: 2682-2918 or a sequence with less than 3 substitutions in the three CDRs of the sequences set forth in any one or more of SEQ ID NOS: 2682-2918, or a sequence with less than three substitutions in the CDR1, CDR2 and CDR3 that belong to a vH and are presented in SEQ ID NO: 11593-11829, 11830-12066, 12067-12303, respectively, or a sequence that binds to the same target antigens or the same epitopes on the target antigens as a sequence set forth in any one or more of SEQ ID NOS: 2682-2918 and which encodes a polypeptide that binds to its antigen; and
(i.) a complementary light chain variable region (vL) of the antibody comprising a sequence as set forth in any one of SEQ ID NO: 2440-2676 or sequences with at least 80% identity to sequences set forth in any one or more of SEQ ID NOS: 2440-2676 or a sequence with at least 80% identity in the three complementarity determining regions (CDRs) to the sequences set forth in any one or more of SEQ ID NOS: 2440-2676 or a sequence with less than 3 substitutions in the three CDRs of the sequences set forth in any one or more of SEQ ID NOS: 12440-2676 or a sequence with less than three substitutions in the CDR1, CDR2 and CDR3 that belong to a vL and are presented in SEQ ID NO: 10882-11118, 11119-11355 and 11356-11592, respectively, or a sequence that binds to the same target antigens or the same epitopes on the target antigens as a sequence set forth in any one or more of SEQ ID NOS: 2440-2676 and which encodes a polypeptide that binds to its antigen;
(ii) a single chain variable fragment (scFv) comprising a sequence as set forth in any one SEQ ID NO: 2924-3160 or a sequence with at least 80% identity thereto or a sequence with at least 70% identity in the six complementarity determining regions (CDRs) to the sequences set forth in any one or more of SEQ ID NOS: 2924-3160 or a sequence with less than 6 substitutions in the six CDRs of the sequences set forth in any one or more of SEQ ID NOS: 2924-3160 or a sequence with less than three substitutions in the CDR1, CDR2 and CDR3 that belong to a vH comprising a scFV and are presented in SEQ ID NO: 11593-11829, 11830-12066, 12067-12303, respectively and less than three substitutions in the light chain CDR1, CDR2 and CDR3 that belong to a vL comprising a scFv and are presented in SEQ ID NO: 10882-11118, 11119-11355 and 11356-1159 respectively, or a sequence that binds to the same target antigens or the same epitopes on the target antigens as a sequence set forth in any one or more of SEQ ID NOS: 2924-3160 and which encodes a polypeptide that binds to its antigen;
(iii.) a single domain antibody, a vHH domain, a SVH, and/or FHVH domain comprising a sequence as set forth in any one of SEQ ID NO: 3210-3353, 10695-10713 or a sequence with at least 70% identity to a sequence set forth in any one or more of SEQ ID NOS: 3210-3353, 10695-10713 and or a sequence with at least 70% identity in the three complementarity determining regions (CDRs) to the sequences set forth in any one or more of SEQ ID NOS: 3210-3353, 10695-10713 or a sequence with less than 3 substitutions in the three CDRs of the sequences set forth in any one or more of SEQ ID NOS: 3210-3353, 10695-10713 or a sequence that bind to the same target antigens or the same epitopes on the target antigens as a sequence set forth in any one or more of SEQ ID NOS: 3210-3353, 10695-10713 and which encodes a polypeptide that binds to its antigen;
(iv.) a non-immunoglobulin scaffold encoded by a polynucleotide of any one of SEQ ID NOS: 3366-3377 or sequences with at least 70% identity to sequences set forth in any one or more of SEQ ID NOS: 3366-3377 or sequences that bind to the same target antigens or the same epitopes on the target antigens as the sequences set forth in any one or more of SEQ ID NOS: 3366-3377;
(v.) the ligand binding domain of a receptor comprising a sequence as set forth in any one of SEQ ID NO: 3378-3395, 3880, 3882, 3886, 3893, 3896, 3897 or sequences with at least 70% identity thereto and which encodes a polypeptide that binds to its cognate;
(vi.) the receptor binding domain of a ligand comprising a sequence as set forth in any one of SEQ ID NO: 3396-3406, 10786-10787 or sequences with at least 70% identity thereto and which encodes a polypeptide that binds to its cognate;
(vii.) an adaptor binding domain comprising a sequence as set forth in any one of SEQ ID NO: 3407-3435, 10771-10780 or sequences with at least 70% identity thereto and which encodes a polypeptide that binds to its adaptor;
an autoantigen comprising a sequence as set forth in any one of SEQ ID NO 10788-10791 or sequences with at least 70% identity thereto and which encodes a polypeptide that binds to its autoantibody or autoantibody producing cells;
(ix.) a TCR variable region (Va, Vb, Vg or Vd) comprising a sequence as set forth in any of SEQ ID NOs: 3357-3364, 9606-9614, 10781-10782 or sequences with at least 70% identity thereto or sequences with at least 70% identity in the three complementarity determining regions (CDRs) to the sequences set forth in any one or more of SEQ ID NOS: 3357-3364, 9606-9614, 10781-10782 or sequences with less than 3 substitutions in the three CDRs of the sequences set forth in any one or more of SEQ ID NOS: 3357-3364, 9606-9614, 10781-10782 or sequences that bind to the same target antigens or the same epitopes on the target antigens as the sequences set forth in any one or more of SEQ ID NOS: 3357-3364, 9606-9614, 10781-10782 and which encodes a polypeptide that binds to its antigen; and
(x.) a single variable TCR domain (svd-TCR) comprising a sequence as set forth in any of SEQ ID NOs: 9613-9614 or sequences with at least 70% identity thereto or sequences with 70-99% identity in the three complementarity determining regions (CDRs) to the sequences set forth in any one or more of SEQ ID NOS: 9613-9614 or sequences with less than 3 substitutions in the three CDRs of the sequences set forth in any one or more of SEQ ID NOS: 9613-9614 or sequences that bind to the same target antigens or the same epitopes on the target antigens as the sequences set forth in any one or more of SEQ ID NOS: 9613-9614 and which encodes a polypeptide that binds to its antigen;
(xi.) a naturally occurring receptor and/or the signaling adaptor or a fragment thereof comprising a sequence selected from SEQ ID NO: 3743-3966, 3385, 3394, 7818-7822, 9633-9859 or a sequence with 70% homology to a sequence thereto;
(xii.) a hinge domain, transmembrane domain and/or cytosolic domains of naturally occurring receptor and/or the signaling adaptor comprising a sequence selected from SEQ ID NO: 9669-9704, 3813, 8721, 8733 and 8746 or a sequence with 70% homology to a sequence thereto;
(xiii.) a membrane associated domain of a naturally occurring receptor and/or a signaling adaptor comprising a sequence selected from SEQ ID NO: 3914-3928, 9741-9776, 9852-9855 or a sequence with 70% homology to a sequence thereto;
(xiv.) a cytosolic domain of a naturally occurring receptor and/or a signaling adaptor comprising a sequence selected from SEQ ID NO: 3944-3958, 9777-9812, 9856-9859 or a sequence with 70% homology to a sequence thereto;
(xv.) an activation domain of a naturally occurring receptor and/or a signaling adaptor comprising a sequence selected from SEQ ID NO: 9856-9859 and 9777 or a sequence with 70% homology to a sequence thereto;
(xvi.) a co-stimulatory domain comprising a sequence selected from SEQ ID NO: 9807-9810 or a sequence with 70% homology to a sequence thereto;
(xvii.) a leader sequence or a signal peptide that is present at the N-terminal of each polypeptide chain and optionally comprising a sequence selected from the group consisting of SEQ ID NO:2425-2430.

73-92. (canceled)

93. The recombinant polynucleotide encoding the SAR of claim 1, further expressing an accessory module comprising a polypeptide that is selected from the group consisting of:
a) a cytokine or a variant thereof optionally comprising a sequence with SEQ ID NO:7833-7842 or a variant with up to 70% sequence homology thereto;
b) membrane-anchored cytokine or a membrane anchored cytokine with epitope tags or a variant thereof optionally comprising a sequence with SEQ ID NO:7825-7832 or a variant with up to 70% sequence homology thereto;
c) a multi-purpose switch that serves as a suicide, survival and marker function optionally comprising a sequence with SEQ ID NO: 7843-7850 or a variant with up to 70% sequence homology thereto;
d) a multi-purpose switch having the formula: SP-D1-L1-D2-L2-D3-L3-D4: wherein
SP is an optional signal peptide that allows cell surface transport of the multipurpose switch and is cleaved to yield the mature peptide,
D1 is receptor binding domain which binds to a receptor that promotes cell survival,
D2 is a marker/suicide domain,
D3 is a hinge domain/stalk domain that allows the D1 and D2 domains to be projected away from the surface of the target cell,
D4 is a membrane associating domain that anchors the multi-purpose switch to the cell membrane,
and L1, L2 and L3 are optional linker.
e) a signaling adaptor molecule optionally selected from the group of CD3ƒ, FcRγ, DAP10 and DAP12;
f) a kill-switch;
g) truncated epidermal growth factor receptor (tEGFR), truncated epidermal growth factor receptor viii (tEGFRviii), truncated CD30 (tCD30), truncated BCMA (tBCMA), truncated CD19 (tCD19), CD34, thymidine kinase, cytosine deaminase, nitroreductase, xanthine-guanine phosphoribosyl transferase, human caspase 8, human caspase 9, inducible caspase 9 (icaspase9), purine nucleoside phosphorylase, linamarase/linamarin/glucose oxidase, deoxyribonucleoside kinase, horseradish peroxidase (HRP)/indole-3-acetic (IAA), Gamma-glutamylcysteine synthetase, CD20/alphaCD20, CD34/thymidine kinase chimera, dox-dependent caspase-2, mutant thymidine kinase (HSV-TKSR39), AP1903/Fas system, a chimeric cytokine receptor (CCR), a selection marker, a multi-purpose switch, vFLIP-K13, vFLIP-MC159, 4-1BBL-CD40L, dihydroxyfolate receptor (DHFR), mutant DHFR, methylated-DNA-protein-cysteine methyltransferase, inosine monophosphate dehydrogenase II (IMDHP2), puromycin acetyle transferase (PAC), blasticidin-resistance gene, mutant calcinueurin a/b (Can/b), CNa12, CNb30, of PDL1, PDL2, CD80, CD86, crmA, p35, hNEMO-K277A (or NEMO-K277A), hNEMO-K277A-delta-V249-K555, mNEMO-K270A, K13-opt, IKK2-S177E-S181E (or IKK2-SS/EE), IKK1-S176E-S180E (or IKK1-SS/EE), MyD88-L265P, TCL-1a, MTCP-1, CMV-141, vFLIP-K13, MC159, cFLIP-L/MRITα, cFLIP-p22, HTLV1 Tax, HTLV2 Tax, HTLV2 Tax-RS mutant, FKBPx2-K13, FKBPx2-HTLV2-Tax, FKBPx2-HTLV2-Tax-RS, IL6R-304-vHH-Alb8-vHH, IL12f, PD1-4H1 scFV, PD1-5C4 scFV, PD1-4H1-Alb8-vHH, PD1-5C4-Alb8-vHH, CTLA4-Ipilimumab-scFv, CTLA4-Ipilimumab-Alb8-vHH, IL6-19A-scFV, IL6-19A-scFV-Alb8-vHH, sHVEM, sHVEM-Alb8-vHH, hTERT, Fx06, shRNA targeting Brd4, IgSP-[hTRAC-opt2], IgSP-[hTRBC-opt21, IL2-tBCMA, IL15-tBCMA, IL2-RQR, IL15-RQR, NKG2C, CD94, DAP10, DAP12, CD3R, CD3γ, CD3δ, CD3ƒ, FcRγ, IL2, IL-7, IL-15, IL12f, IL21, membrane anchored form of IL2 or combination thereof;
h) an agent that provides costimulation to SAR expressing cell;
i) an agent that provides costimulation to SAR expressing cell in an inducible manner;
j) an agent that modulate the activity of SAR expressing cells;
k) an agent that enhance or regulate the activity of SAR expressing cells;
l) an agent that promote the proliferation and/or persistence of SAR-expressing cells.

94-117. (canceled)

118. The recombinant SAR polypeptide or polypeptide heterodimer encoded by the at least one recombinant polynucleotide of claim 1 or 2 and optionally co-expressed with the one or more accessory modules of claim 93.

119-126. (canceled)

127. An effector cell or a stem cell comprising the at least one recombinant polynucleotide of claim 1, the SAR polypeptide or polypeptide heterodimer of claim 118, and the one or more optional accessory modules of claim 93.

128-136. (canceled)

137. The effector cell or stem cell of claim 127, wherein the cell has one or more of the characteristics selected from the group consisting of
a) the effector cell or the stem cell cell is a α/β T cell, γ/δ T cell, CD8⁺ T cell, a CD4⁺ T cell, a memory T cell, naïve T cell, T stem cell, a Treg cell, natural killer T (NKT) cell, iNKT (innate natural killer cell), NK cell, g-NK cell, memory like NK cells, cytokine induced killer cell (CIK), iPSC, a modified HLA deficient iPSC, iPSC-derived NK cell, iPSC-derived T cell, B cell, a macrophage/monocyte, granulocyte, a dendritic cell, an immortalized cell line, an immortalized NK cell line, NK92 cell line, NK92MI cell line, YTS cell or derivative thereof
(b) the effector cell or the stem cell lacks expression or has low expression of a functional TCR, a functional HLA, P2 macroglobulin, TAP1, TAP2. tapasin, NLRC5, CIITA, RFXANK, CIITA, RFX5, RFXAP, TCRα or R constant region, NKG2A, NKG2D, CD3ε, CD5, CD52, CD33, CD123, CLL-1, CIS, CBL-B, SOCS2, PD1, CTLA4, LAG3, TIM3, TIGIT, or any gene in the chromosome 6p21 region; and/or
(c) the effector cell or the stem cell shows introduced or increased expression in at least one of HLA-E, 41BBL, CD3ε, CD3γ, CD3δ, CD3ƒ, FcRγ, DAP10, DAP12, CD4, CD8, CD16, CD47, CD94, CD113, CD131, CD137, CD80, PDL1, A2AR, Fc receptor, an engager, or surface triggering receptor for coupling with bi- or multi-specific or universal engagers; and/or
(d) the effector cell or the stem cell is modified to block or decrease the expression of a first endogenous TCR subunit and/or a second endogenous TCR subunit; and/or
(e) the effector cell or the stem cell does not express a T cell receptor (TCR) and/or does not express CD3F, CD3γ or CD3δ and the cell is modified by recombinant expression to express a recombinant double chain TCR exogenous to the cell, wherein said recombinant double chain TCR is a SAR which comprises a TCR antigen-recognition domain comprising a) Va and Vβ domains or b) Vγ and VS domains and a non-T cell receptor module (NTCRM). (f) the effector cell or the stem cell comprises a TCR antigen recognition motif that is operationally linked via optional linkers to a non-T cell receptor module (NTCRM) comprising a first MAM and a second MAM derived from non-T cell receptors and/or signaling adaptors and further comprising optional cytosolic co-stimulatory domains (g) the effector cell or the stem cell comprises a plurality of SAR polypeptides; and/or
(h) the effector cell or the stem cell comprises a plurality of SAR polypeptides that target the same antigen or target a different antigen than at least one other SAR polypeptide; and/or
(i) the effector cell or the stem cell comprises a plurality of SAR polypeptides that comprise a different binding affinity for an antigen than at least one other SAR polypeptide; and/or
(j) the effector cell or the stem cell comprises a plurality of SAR polypeptides wherein at least one SAR polypeptide of the plurality of SAR polypeptides comprises a different naturally occurring receptor or a signaling adaptor than at least one other SAR polypeptide; and/or
(k) the effector cell or the stem cell comprises a plurality of SAR polypeptides wherein the least one SAR polypeptide of the plurality of SAR polypeptides has a different extracellular domain, transmembrane domain, cytosolic domain than at least one other SAR polypeptide; and/or
(l) the effector cell or the stem cell comprises a plurality of SAR polypeptides wherein the least one SAR polypeptide of the plurality of SAR polypeptides is an activating receptor and at least one other SAR polypeptide is an inhibitory receptor; and/or
(m) the effector cell or the stem cell comprises a plurality of SAR polypeptides wherein the two or more SAR polypeptide of the plurality of SAR polypeptides are activating receptors or two or more SAR polypeptide of the plurality of SAR polypeptides are inhibitory receptors; and/or
(n) the effector cell or the stem cell comprises a plurality of SAR polypeptides wherein the two or more SAR polypeptide of the plurality of SAR polypeptides recruit different signaling adaptors and/or activate different signal transduction pathways; and/or
(o) the effector cell or the stem cell is a non-T cell but possesses T cell like antigen recognition; and/or
(p) the effector cell or the stem cell lacks the functional expression of CD3γ, CD3δ and/or CD3ε chains but possesses T cell like antigen recognition; and/or
(q) the cell is a non-T cell but can induce T cell like signaling upon antigen recognition; and/or
(r) the effector cell or the stem cell lacks the functional expression of CD3γ, CD3δ and/or CD3ε chains but can induce T cell like signaling upon antigen recognition; and/or
(s) the cell is a non-T cell but can bind to a peptide in complex with an MHC (HLA) molecule; and/or
(t) the effector cell or the stem cell is a non-T cell but can initiate at least one signaling pathway when bound by a peptide/NMC complex; and/or
(u) the effector cell or the stem cell expresses a cytokine or a variant thereof optionally comprising a sequence with SEQ ID NO:7833-7842 or a variant with up to 70% sequence homology thereto; and/or
(v) the effector cell or the stem cell expresses a membrane-anchored cytokine or a membrane anchored cytokine with epitope tags or a variant thereof optionally comprising a sequence with SEQ ID NO:7825-7832 or a variant with up to 70% sequence homology thereto; and/or
(w) the effector cell or the stem cell expresses a multi-purpose switch that serves as a suicide, survival and marker function optionally comprising a sequence with SEQ ID NO: 7843-7850 or a variant with up to 70% sequence homology thereto; and/or
(x) the effector cell or the stem cell 1 expresses one or more accessory modules.

138-147. (canceled)

148. A method of providing anti-disease immunity in a subject comprising administering to the subject an effective amount of the immune effector cell or the stem cell that can give rise to an immune effector cell of claim 127, wherein optionally the immune effector cell is an autologous T cell, an allogeneic T cell, an autologous NK cell, an allogeneic NK cell, an autologous macrophage, an allogeneic macrophage, an autologous granulocyte, an allogeneic granulocyte, an autologous dendritic cell, an allogeneic dendritic cell, an autologous hematopoietic stem, an allogeneic hematopoietic stem cell, an autologous iPSC, or an allogeneic iPSC that can give rise to an effector cell,
wherein optionally the disease is selected from the group consisting of: a cancer; a viral disease, an autoimmune disease; a degenerative disease; or an infection.

149-165. (canceled)

166-245. (canceled)

246. The at least one recombinant polynucleotide encoding the SAR of claim 1, wherein the sequence of the encoded two polypeptide chains of the SAR is identical to the sequence of the two polypeptide chains of a SAR selected from the group consisting of SEQ ID NO: 6283-6294, 6315-6326, 6347-6358, 6379-6390, 6411-6422, 6443-6454, 6475-6486, 6507-6518, 6539-6550, 6571-6582, 6603-6614, 6635-6646, 6667-6678, 6699-6710, 6731-6742, 6763-6774, 6795-6806, 6827-6839, 6859-6870, 6891-6902, 6923-6934, 6955-6966, 6987-6998, 7019-7030, 7051-7062, 7083-7094, 10338, 10345, 10347, 10350-10353, 10355-10362, 10364, 10369-10375, 10378-10379, 10387-10392, 10394, 10396-7, 10421-10425, 10427-10428, 10466-10694, 10838-10841, or a sequence with at least 75% identity to an amino acid sequence encoding the synthetic immune receptor set forth in any one of the above.
